SEQUENCE LISTING (334 sequences)

SEQ ID NO: 1 - DNA, 2835 bp, synthetic construct
  source 1..2835: mol_type = other DNA
atggcactgccagtgaccgccctgctgctgcctctggccctgctgctgcacgcagccagaccccaggtgcagctgcagcagtctggacctgagctggtgaagccaggagcctccgtgaagatgtcttgcaaggccagcggctacaccttcacatcttatcacatccagtgggtgaagcagcggcccggacagggcctggagtggatcggatggatctacccaggcgacggctccacacagtataacgagaagttcaagggcaagaccacactgaccgccgataagagcagcagcaccgcctacatgctgctgagcagcctgaccagcgaggacagcgccatctacttttgcgccagggagggcacatactatgctatggactattggggccagggcaccagcgtgacagtgtctagcggaggaggaggctccggaggaggaggctctggcggcggcggcagcgacgtgctgatgacccagacaccactgagcctgcccgtgagcctgggcgatcaggtgagcatctcctgtagatcctctcagagcatcgtgcactccaacggcaatacctacctggagtggtatctgcagaagccaggccagtcccccaagctgctgatctataaggtgtctaatcggttcagcggcgtgcctgacagattttctggcagcggctccggcaccgacttcaccctgaagatcagccgggtggaggcagaggatctgggcgtgtactattgtttccagggctcccacgtgccacgcacctttggcggcggtaccaagctggagatcaagattgaagttatgtatcctcctccttacctagacaatgagaagagcaatggaaccattatccatgtgaaagggaaacacctttgtccaagtcccctatttcccgggccttcgaagcccttttgggtgctggtggtggttggtggagtcctggcttgctatagcttgctagtaacagtagcgtttattattttctgggtgtgcagcagggccgcccgcggcaccatcggcgccaggcgcacaggccagcctctgaaggaggacccttccgccgtgccagtgttctctgtggactacggcgagctggattttcagtggcgggagaaaaccccagagccacctgtgccctgcgtgcctgagcagaccgagtatgccacaatcgtgtttccatccggaatgggcacaagctcccctgcaaggagaggcagcgccgacggaccacggtccgcccagccactgcggcccgaggatggccactgttcttggcccctgcggagaaagcgtggatccggggaaggccgaggctcccttctaacatgtggagatgtcgaggaaaaccctggccctatggcgctgccagtcactgcattgttattgcctctggccctgcttctccatgcggcgcgcccagaagtgcagctggtcgagagcggaggcggactggttcaacccggaggcagcttgagactgtcctgcgcggccagcggcttcaacatcaaggatacctatatccactgggtgaggcaggctccaggaaagggcctggagtgggtggcaaggatttaccctactaatggatatacacgctacgctgattccgtgaagggacgctttacaatctcagcagatacatccaaaaacacggcctatttacagatgaatagtttgcgggccgaagacacggctgtatactattgttctcggtgggggggcgatggattttatgcgatggattactggggccagggcaccctggtaaccgtgtcaagcggctcaacatccgggtccggtaagccgggctccggcgaggggtctacaaagggagatatacagatgacacagtcccccagttccctgtccgcctcagtgggagaccgagtgacgattacctgtcgtgccagccaggacgtcaataccgccgtcgcttggtatcagcaaaaaccaggcaaggccccgaaactattgatctacagtgcctcttttctgtactccggggtgccgagcagatttagtggctccaggagcggaaccgatttcaccctaaccatttccagtttgcagccagaggatttcgcgacctattactgccagcaacactacaccacaccgccaactttcggacaaggaaccaaggttgaaatcaaaactacgaccccagcacctagacctcccaccccagctccaactatagcttcccagccattgtctctccggccagaggcgtgtcgaccagccgctggaggggccgttcatacaagaggactcgatttcgcttgcgatatctacatatgggcccctcttgccgggacatgcggtgtcctgcttctaagcttggttattaccctctattgcaaacgcggccgcaagaaactgctctacatctttaaacagccgttcatgaggcctgtgcagacaacgcaggaagaggatggctgtagttgtcggtttccggaagaggaagaggggggctgcgagttgcgtgtcaaattttctcggtctgccgacgcccccgcgtaccagcaagggcagaaccagctttataatgagctgaatcttggacgacgggaggaatatgacgtgcttgacaagaggcgaggtagggaccctgagatggggggaaaacctcggaggaaaaacccacaggaaggcctgtataacgaactgcagaaggacaagatggctgaagcctactctgagattggaatgaaaggggaacgcagacgcggcaagggccatgatggcctctaccaaggtctaagcactgccaccaaggacacctatgacgcactccacatgcaagctctacctccccgttgataa

SEQ ID NO: 2 - protein, 943 aa, synthetic construct
MALPVTALLLPLALLLHAARPQVQLQQSGPELVKPGASVKMSCKASGYTFTSYHIQWVKQRPGQGLEWIGWIYPGDGSTQYNEKFKGKTTLTADKSSSTAYMLLSSLTSEDSAIYFCAREGTYYAMDYWGQGTSVTVSSGGGGSGGGGSGGGGSDVLMTQTPLSLPVSLGDQVSISCRSSQSIVHSNGNTYLEWYLQKPGQSPKLLIYKVSNRFSGVPDRFSGSGSGTDFTLKISRVEAEDLGVYYCFQGSHVPRTFGGGTKLEIKIEVMYPPPYLDNEKSNGTIIHVKGKHLCPSPLFPGPSKPFWVLVVVGGVLACYSLLVTVAFIIFWVCSRAARGTIGARRTGQPLKEDPSAVPVFSVDYGELDFQWREKTPEPPVPCVPEQTEYATIVFPSGMGTSSPARRGSADGPRSAQPLRPEDGHCSWPLRRKRGSGEGRGSLLTCGDVEENPGPMALPVTALLLPLALLLHAARPEVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSGSTSGSGKPGSGEGSTKGDIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 3 - DNA, 2829 bp, synthetic construct
  source 1..2829: mol_type = other DNA
atggcactgccagtgaccgccctgctgctgcctctggccctgctgctgcacgcagccagaccccaggtgcagctgcagcagtctggacctgagctggtgaagccaggagcctccgtgaagatgtcttgcaaggccagcggctacaccttcacatcttatcacatccagtgggtgaagcagcggcccggacagggcctggagtggatcggatggatctacccaggcgacggctccacacagtataacgagaagttcaagggcaagaccacactgaccgccgataagagcagcagcaccgcctacatgctgctgagcagcctgaccagcgaggacagcgccatctacttttgcgccagggagggcacatactatgctatggactattggggccagggcaccagcgtgacagtgtctagcggaggaggaggctccggaggaggaggctctggcggcggcggcagcgacgtgctgatgacccagacaccactgagcctgcccgtgagcctgggcgatcaggtgagcatctcctgtagatcctctcagagcatcgtgcactccaacggcaatacctacctggagtggtatctgcagaagccaggccagtcccccaagctgctgatctataaggtgtctaatcggttcagcggcgtgcctgacagattttctggcagcggctccggcaccgacttcaccctgaagatcagccgggtggaggcagaggatctgggcgtgtactattgtttccagggctcccacgtgccacgcacctttggcggcggtaccaagctggagatcaagattgaagttatgtatcctcctccttacctagacaatgagaagagcaatggaaccattatccatgtgaaagggaaacacctttgtccaagtcccctatttcccgggccttcgaagcccttttgggtgctggtggtggttggtggagtcctggcttgctatagcttgctagtaacagtagcgtttattattttctgggtgtgcagcagggccgcccgcggcaccatcggcgccaggcgcacaggccagcctctgaaggaggacccttccgccgtgccagtgttctctgtggactacggcgagctggattttcagtggcgggagaaaaccccagagccacctgtgccctgcgtgcctgagcagaccgagtatgccacaatcgtgtttccatccggaatgggcacaagctcccctgcaaggagaggcagcgccgacggaccacggtccgcccagccactgcggcccgaggatggccactgttcttggcccctgcggagaaagcgtggatccggggaaggccgaggctcccttctaacatgtggagatgtcgaggaaaaccctggccctatggcgctgccagtcactgcattgttattgcctctggccctgcttctccatgcggcgcgcccacaagtgcagctgaaacagagcggaccaggactggttcaacccagccagagcttgagcatcacgtgcacggttagcggcttcagtctgaccaattatggtgtgcactgggtgaggcagtctccaggaaagggcctggagtggcttggagtcatttggagcggtgggaatacagattacaatacaccttttacgtcacgtctctccattaacaaggacaactccaaatcccaagtatttttcaaaatgaatagcctgcagagtaatgataccgccatctattactgtgcacgagctttgacatattacgactatgaatttgcctattggggtcaaggcacgctggtgaccgtatcaggctcaacatccgggtccggtaagccgggctccggcgaggggtctacaaagggagacatccttctgacacagagccccgtgatcctgtccgtgtcccccggcgagagagtatcattttcctgtagggcttctcagagcatcggaacaaatatccactggtatcagcaacggactaacggatcacctcgcctgctcataaagtacgccagtgaatctattagtggcataccgagccgcttcagcgggagtggctccggcacagactttactctgagtataaattccgtggaatctgaggacatcgcggactattactgccagcaaaacaataactggcccaccacgttcggcgcgggaactaaactagaactaaagactacgaccccagcacctagacctcccaccccagctccaactatagcttcccagccattgtctctccggccagaggcgtgtcgaccagccgctggaggggccgttcatacaagaggactcgatttcgcttgcgatatctacatatgggcccctcttgccgggacatgcggtgtcctgcttctaagcttggttattaccctctattgcaaacgcggccgcaagaaactgctctacatctttaaacagccgttcatgaggcctgtgcagacaacgcaggaagaggatggctgtagttgtcggtttccggaagaggaagaggggggctgcgagttgcgtgtcaaattttctcggtctgccgacgcccccgcgtaccagcaagggcagaaccagctttataatgagctgaatcttggacgacgggaggaatatgacgtgcttgacaagaggcgaggtagggaccctgagatggggggaaaacctcggaggaaaaacccacaggaaggcctgtataacgaactgcagaaggacaagatggctgaagcctactctgagattggaatgaaaggggaacgcagacgcggcaagggccatgatggcctctaccaaggtctaagcactgccaccaaggacacctatgacgcactccacatgcaagctctacctccccgttgataa

SEQ ID NO: 4 - protein, 941 aa, synthetic construct
MALPVTALLLPLALLLHAARPQVQLQQSGPELVKPGASVKMSCKASGYTFTSYHIQWVKQRPGQGLEWIGWIYPGDGSTQYNEKFKGKTTLTADKSSSTAYMLLSSLTSEDSAIYFCAREGTYYAMDYWGQGTSVTVSSGGGGSGGGGSGGGGSDVLMTQTPLSLPVSLGDQVSISCRSSQSIVHSNGNTYLEWYLQKPGQSPKLLIYKVSNRFSGVPDRFSGSGSGTDFTLKISRVEAEDLGVYYCFQGSHVPRTFGGGTKLEIKIEVMYPPPYLDNEKSNGTIIHVKGKHLCPSPLFPGPSKPFWVLVVVGGVLACYSLLVTVAFIIFWVCSRAARGTIGARRTGQPLKEDPSAVPVFSVDYGELDFQWREKTPEPPVPCVPEQTEYATIVFPSGMGTSSPARRGSADGPRSAQPLRPEDGHCSWPLRRKRGSGEGRGSLLTCGDVEENPGPMALPVTALLLPLALLLHAARPQVQLKQSGPGLVQPSQSLSITCTVSGFSLTNYGVHWVRQSPGKGLEWLGVIWSGGNTDYNTPFTSRLSINKDNSKSQVFFKMNSLQSNDTAIYYCARALTYYDYEFAYWGQGTLVTVSGSTSGSGKPGSGEGSTKGDILLTQSPVILSVSPGERVSFSCRASQSIGTNIHWYQQRTNGSPRLLIKYASESISGIPSRFSGSGSGTDFTLSINSVESEDIADYYCQQNNNWPTTFGAGTKLELKTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 5 - DNA, 2835 bp, synthetic construct
  source 1..2835: mol_type = other DNA
atggcactgccagtgaccgccctgctgctgcctctggccctgctgctgcacgcagccagaccccaagtgcaactagtccaatcaggtggaggcgtcgtgcaacctggagggtccctccgcgttagctgcgccgcatcaggcgttaccttgtcagactacggcatgcattgggttaggcaagcccccggcaaggggctcgaatggatggctttcattcggaatgacgggagcgataaatattacgcggattcagttaaagggcggttcaccatcagccgcgacaatagcaaaaagacggtctccttacagatgtccagcttgcgggccgaagacacggctgtatactattgtgctaaaaatggcgagagcggccccctggattactggtactttgacctgtggggcagaggcaccctggtcacggtgtcctctggaggaggaggctccggaggaggaggctctggcggcggcggcagcgacattgtaatgacccagtcaccctccttccttagtgcctcagtcggagaccgcgtgactatcacttgtcgtgcctcacacggaattaataactacctcgcttggtatcagcaaaaaccaggcaaggccccgaaactattgatctacgccgcatctactctgcagagcggagtaccgagcagatttagtggttccggcagcggaaccgagttcaccctaaccatttccagtttgcagccagaggatttcgcgacctattactgccagcaatacgattcatacccgccaactttcggaagaggtaccaaggttgaaatcaagattgaagttatgtatcctcctccttacctagacaatgagaagagcaatggaaccattatccatgtgaaagggaaacacctttgtccaagtcccctatttcccgggccttcgaagcccttttgggtgctggtggtggttggtggagtcctggcttgctatagcttgctagtaacagtagcgtttattattttctgggtgtgcagcagggccgcccgcggcaccatcggcgccaggcgcacaggccagcctctgaaggaggacccttccgccgtgccagtgttctctgtggactacggcgagctggattttcagtggcgggagaaaaccccagagccacctgtgccctgcgtgcctgagcagaccgagtatgccacaatcgtgtttccatccggaatgggcacaagctcccctgcaaggagaggcagcgccgacggaccacggtccgcccagccactgcggcccgaggatggccactgttcttggcccctgcggagaaagcgtggatccggggaaggccgaggctcccttctaacatgtggagatgtcgaggaaaaccctggccctatggcgctgccagtcactgcattgttattgcctctggccctgcttctccatgcggcgcgcccagaagtgcagctggtcgagagcggaggcggactggttcaacccggaggcagcttgagactgtcctgcgcggccagcggcttcaacatcaaggatacctatatccactgggtgaggcaggctccaggaaagggcctggagtgggtggcaaggatttaccctactaatggatatacacgctacgctgattccgtgaagggacgctttacaatctcagcagatacatccaaaaacacggcctatttacagatgaatagtttgcgggccgaagacacggctgtatactattgttctcggtgggggggcgatggattttatgcgatggattactggggccagggcaccctggtaaccgtgtcaagcggctcaacatccgggtccggtaagccgggctccggcgaggggtctacaaagggagatatacagatgacacagtcccccagttccctgtccgcctcagtgggagaccgagtgacgattacctgtcgtgccagccaggacgtcaataccgccgtcgcttggtatcagcaaaaaccaggcaaggccccgaaactattgatctacagtgcctcttttctgtactccggggtgccgagcagatttagtggctccaggagcggaaccgatttcaccctaaccatttccagtttgcagccagaggatttcgcgacctattactgccagcaacactacaccacaccgccaactttcggacaaggaaccaaggttgaaatcaaaactacgaccccagcacctagacctcccaccccagctccaactatagcttcccagccattgtctctccggccagaggcgtgtcgaccagccgctggaggggccgttcatacaagaggactcgatttcgcttgcgatatctacatatgggcccctcttgccgggacatgcggtgtcctgcttctaagcttggttattaccctctattgcaaacgcggccgcaagaaactgctctacatctttaaacagccgttcatgaggcctgtgcagacaacgcaggaagaggatggctgtagttgtcggtttccggaagaggaagaggggggctgcgagttgcgtgtcaaattttctcggtctgccgacgcccccgcgtaccagcaagggcagaaccagctttataatgagctgaatcttggacgacgggaggaatatgacgtgcttgacaagaggcgaggtagggaccctgagatggggggaaaacctcggaggaaaaacccacaggaaggcctgtataacgaactgcagaaggacaagatggctgaagcctactctgagattggaatgaaaggggaacgcagacgcggcaagggccatgatggcctctaccaaggtctaagcactgccaccaaggacacctatgacgcactccacatgcaagctctacctccccgttgataa

SEQ ID NO: 6 - protein, 943 aa, synthetic construct
MALPVTALLLPLALLLHAARPQVQLVQSGGGVVQPGGSLRVSCAASGVTLSDYGMHWVRQAPGKGLEWMAFIRNDGSDKYYADSVKGRFTISRDNSKKTVSLQMSSLRAEDTAVYYCAKNGESGPLDYWYFDLWGRGTLVTVSSGGGGSGGGGSGGGGSDIVMTQSPSFLSASVGDRVTITCRASHGINNYLAWYQQKPGKAPKLLIYAASTLQSGVPSRFSGSGSGTEFTLTISSLQPEDFATYYCQQYDSYPPTFGRGTKVEIKIEVMYPPPYLDNEKSNGTIIHVKGKHLCPSPLFPGPSKPFWVLVVVGGVLACYSLLVTVAFIIFWVCSRAARGTIGARRTGQPLKEDPSAVPVFSVDYGELDFQWREKTPEPPVPCVPEQTEYATIVFPSGMGTSSPARRGSADGPRSAQPLRPEDGHCSWPLRRKRGSGEGRGSLLTCGDVEENPGPMALPVTALLLPLALLLHAARPEVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSGSTSGSGKPGSGEGSTKGDIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 7 - DNA, 2829 bp, synthetic construct
  source 1..2829: mol_type = other DNA
atggcactgccagtgaccgccctgctgctgcctctggccctgctgctgcacgcagccagaccccaagtgcaactagtccaatcaggtggaggcgtcgtgcaacctggagggtccctccgcgttagctgcgccgcatcaggcgttaccttgtcagactacggcatgcattgggttaggcaagcccccggcaaggggctcgaatggatggctttcattcggaatgacgggagcgataaatattacgcggattcagttaaagggcggttcaccatcagccgcgacaatagcaaaaagacggtctccttacagatgtccagcttgcgggccgaagacacggctgtatactattgtgctaaaaatggcgagagcggccccctggattactggtactttgacctgtggggcagaggcaccctggtcacggtgtcctctggaggaggaggctccggaggaggaggctctggcggcggcggcagcgacattgtaatgacccagtcaccctccttccttagtgcctcagtcggagaccgcgtgactatcacttgtcgtgcctcacacggaattaataactacctcgcttggtatcagcaaaaaccaggcaaggccccgaaactattgatctacgccgcatctactctgcagagcggagtaccgagcagatttagtggttccggcagcggaaccgagttcaccctaaccatttccagtttgcagccagaggatttcgcgacctattactgccagcaatacgattcatacccgccaactttcggaagaggtaccaaggttgaaatcaagattgaagttatgtatcctcctccttacctagacaatgagaagagcaatggaaccattatccatgtgaaagggaaacacctttgtccaagtcccctatttcccgggccttcgaagcccttttgggtgctggtggtggttggtggagtcctggcttgctatagcttgctagtaacagtagcgtttattattttctgggtgtgcagcagggccgcccgcggcaccatcggcgccaggcgcacaggccagcctctgaaggaggacccttccgccgtgccagtgttctctgtggactacggcgagctggattttcagtggcgggagaaaaccccagagccacctgtgccctgcgtgcctgagcagaccgagtatgccacaatcgtgtttccatccggaatgggcacaagctcccctgcaaggagaggcagcgccgacggaccacggtccgcccagccactgcggcccgaggatggccactgttcttggcccctgcggagaaagcgtggatccggggaaggccgaggctcccttctaacatgtggagatgtcgaggaaaaccctggccctatggcgctgccagtcactgcattgttattgcctctggccctgcttctccatgcggcgcgcccacaagtgcagctgaaacagagcggaccaggactggttcaacccagccagagcttgagcatcacgtgcacggttagcggcttcagtctgaccaattatggtgtgcactgggtgaggcagtctccaggaaagggcctggagtggcttggagtcatttggagcggtgggaatacagattacaatacaccttttacgtcacgtctctccattaacaaggacaactccaaatcccaagtatttttcaaaatgaatagcctgcagagtaatgataccgccatctattactgtgcacgagctttgacatattacgactatgaatttgcctattggggtcaaggcacgctggtgaccgtatcaggctcaacatccgggtccggtaagccgggctccggcgaggggtctacaaagggagacatccttctgacacagagccccgtgatcctgtccgtgtcccccggcgagagagtatcattttcctgtagggcttctcagagcatcggaacaaatatccactggtatcagcaacggactaacggatcacctcgcctgctcataaagtacgccagtgaatctattagtggcataccgagccgcttcagcgggagtggctccggcacagactttactctgagtataaattccgtggaatctgaggacatcgcggactattactgccagcaaaacaataactggcccaccacgttcggcgcgggaactaaactagaactaaagactacgaccccagcacctagacctcccaccccagctccaactatagcttcccagccattgtctctccggccagaggcgtgtcgaccagccgctggaggggccgttcatacaagaggactcgatttcgcttgcgatatctacatatgggcccctcttgccgggacatgcggtgtcctgcttctaagcttggttattaccctctattgcaaacgcggccgcaagaaactgctctacatctttaaacagccgttcatgaggcctgtgcagacaacgcaggaagaggatggctgtagttgtcggtttccggaagaggaagaggggggctgcgagttgcgtgtcaaattttctcggtctgccgacgcccccgcgtaccagcaagggcagaaccagctttataatgagctgaatcttggacgacgggaggaatatgacgtgcttgacaagaggcgaggtagggaccctgagatggggggaaaacctcggaggaaaaacccacaggaaggcctgtataacgaactgcagaaggacaagatggctgaagcctactctgagattggaatgaaaggggaacgcagacgcggcaagggccatgatggcctctaccaaggtctaagcactgccaccaaggacacctatgacgcactccacatgcaagctctacctccccgttgataa

SEQ ID NO: 8 - protein, 941 aa, synthetic construct
MALPVTALLLPLALLLHAARPQVQLVQSGGGVVQPGGSLRVSCAASGVTLSDYGMHWVRQAPGKGLEWMAFIRNDGSDKYYADSVKGRFTISRDNSKKTVSLQMSSLRAEDTAVYYCAKNGESGPLDYWYFDLWGRGTLVTVSSGGGGSGGGGSGGGGSDIVMTQSPSFLSASVGDRVTITCRASHGINNYLAWYQQKPGKAPKLLIYAASTLQSGVPSRFSGSGSGTEFTLTISSLQPEDFATYYCQQYDSYPPTFGRGTKVEIKIEVMYPPPYLDNEKSNGTIIHVKGKHLCPSPLFPGPSKPFWVLVVVGGVLACYSLLVTVAFIIFWVCSRAARGTIGARRTGQPLKEDPSAVPVFSVDYGELDFQWREKTPEPPVPCVPEQTEYATIVFPSGMGTSSPARRGSADGPRSAQPLRPEDGHCSWPLRRKRGSGEGRGSLLTCGDVEENPGPMALPVTALLLPLALLLHAARPQVQLKQSGPGLVQPSQSLSITCTVSGFSLTNYGVHWVRQSPGKGLEWLGVIWSGGNTDYNTPFTSRLSINKDNSKSQVFFKMNSLQSNDTAIYYCARALTYYDYEFAYWGQGTLVTVSGSTSGSGKPGSGEGSTKGDILLTQSPVILSVSPGERVSFSCRASQSIGTNIHWYQQRTNGSPRLLIKYASESISGIPSRFSGSGSGTDFTLSINSVESEDIADYYCQQNNNWPTTFGAGTKLELKTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 9 - DNA, 2835 bp, synthetic construct
  source 1..2835: mol_type = other DNA
atggcactgccagtgaccgccctgctgctgcctctggccctgctgctgcacgcagccagaccccaggtgcagctgcagcagtctggacctgagctggtgaagccaggagcctccgtgaagatgtcttgcaaggccagcggctacaccttcacatcttatcacatccagtgggtgaagcagcggcccggacagggcctggagtggatcggatggatctacccaggcgacggctccacacagtataacgagaagttcaagggcaagaccacactgaccgccgataagagcagcagcaccgcctacatgctgctgagcagcctgaccagcgaggacagcgccatctacttttgcgccagggagggcacatactatgctatggactattggggccagggcaccagcgtgacagtgtctagcggaggaggaggctccggaggaggaggctctggcggcggcggcagcgacgtgctgatgacccagacaccactgagcctgcccgtgagcctgggcgatcaggtgagcatctcctgtagatcctctcagagcatcgtgcactccaacggcaatacctacctggagtggtatctgcagaagccaggccagtcccccaagctgctgatctataaggtgtctaatcggttcagcggcgtgcctgacagattttctggcagcggctccggcaccgacttcaccctgaagatcagccgggtggaggcagaggatctgggcgtgtactattgtttccagggctcccacgtgccacgcacctttggcggcggtaccaagctggagatcaagactacgaccccagcacctagacctcccaccccagctccaactatagcttcccagccattgtctctccggccagaggcgtgtcgaccagccgctggaggggccgttcatacaagaggactcgatttcgcttgcgatatctacatatgggcccctcttgccgggacatgcggtgtcctgcttctaagcttggttattaccctctattgctgcagcagggccgcccgcggcaccatcggcgccaggcgcacaggccagcctctgaaggaggacccttccgccgtgccagtgttctctgtggactacggcgagctggattttcagtggcgggagaaaaccccagagccacctgtgccctgcgtgcctgagcagaccgagtatgccacaatcgtgtttccatccggaatgggcacaagctcccctgcaaggagaggcagcgccgacggaccacggtccgcccagccactgcggcccgaggatggccactgttcttggcccctgcggagaaagcgtggatccggggaaggccgaggctcccttctaacatgtggagatgtcgaggaaaaccctggccctatggcgctgccagtcactgcattgttattgcctctggccctgcttctccatgcggcgcgcccagaagtgcagctggtcgagagcggaggcggactggttcaacccggaggcagcttgagactgtcctgcgcggccagcggcttcaacatcaaggatacctatatccactgggtgaggcaggctccaggaaagggcctggagtgggtggcaaggatttaccctactaatggatatacacgctacgctgattccgtgaagggacgctttacaatctcagcagatacatccaaaaacacggcctatttacagatgaatagtttgcgggccgaagacacggctgtatactattgttctcggtgggggggcgatggattttatgcgatggattactggggccagggcaccctggtaaccgtgtcaagcggctcaacatccgggtccggtaagccgggctccggcgaggggtctacaaagggagatatacagatgacacagtcccccagttccctgtccgcctcagtgggagaccgagtgacgattacctgtcgtgccagccaggacgtcaataccgccgtcgcttggtatcagcaaaaaccaggcaaggccccgaaactattgatctacagtgcctcttttctgtactccggggtgccgagcagatttagtggctccaggagcggaaccgatttcaccctaaccatttccagtttgcagccagaggatttcgcgacctattactgccagcaacactacaccacaccgccaactttcggacaaggaaccaaggttgaaatcaaaattgaagttatgtatcctcctccttacctagacaatgagaagagcaatggaaccattatccatgtgaaagggaaacacctttgtccaagtcccctatttcccgggccttcgaagcccttttgggtgctggtggtggttggtggagtcctggcttgctatagcttgctagtaacagtagcgtttattattttctgggtgaaacgcggccgcaagaaactgctctacatctttaaacagccgttcatgaggcctgtgcagacaacgcaggaagaggatggctgtagttgtcggtttccggaagaggaagaggggggctgcgagttgcgtgtcaaattttctcggtctgccgacgcccccgcgtaccagcaagggcagaaccagctttataatgagctgaatcttggacgacgggaggaatatgacgtgcttgacaagaggcgaggtagggaccctgagatggggggaaaacctcggaggaaaaacccacaggaaggcctgtataacgaactgcagaaggacaagatggctgaagcctactctgagattggaatgaaaggggaacgcagacgcggcaagggccatgatggcctctaccaaggtctaagcactgccaccaaggacacctatgacgcactccacatgcaagctctacctccccgttgataa

SEQ ID NO: 10 - protein, 943 aa, synthetic construct
MALPVTALLLPLALLLHAARPQVQLQQSGPELVKPGASVKMSCKASGYTFTSYHIQWVKQRPGQGLEWIGWIYPGDGSTQYNEKFKGKTTLTADKSSSTAYMLLSSLTSEDSAIYFCAREGTYYAMDYWGQGTSVTVSSGGGGSGGGGSGGGGSDVLMTQTPLSLPVSLGDQVSISCRSSQSIVHSNGNTYLEWYLQKPGQSPKLLIYKVSNRFSGVPDRFSGSGSGTDFTLKISRVEAEDLGVYYCFQGSHVPRTFGGGTKLEIKTTTPAPRPPTPAPTIASQPLSLRPEACRPAAGGAVHTRGLDFACDIYIWAPLAGTCGVLLLSLVITLYCCSRAARGTIGARRTGQPLKEDPSAVPVFSVDYGELDFQWREKTPEPPVPCVPEQTEYATIVFPSGMGTSSPARRGSADGPRSAQPLRPEDGHCSWPLRRKRGSGEGRGSLLTCGDVEENPGPMALPVTALLLPLALLLHAARPEVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSGSTSGSGKPGSGEGSTKGDIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKIEVMYPPPYLDNEKSNGTIIHVKGKHLCPSPLFPGPSKPFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 11 - DNA, 2837 bp, synthetic construct
  source 1..2837: mol_type = other DNA
atggcactgccagtgaccgccctgctgctgcctctggccctgctgctgcacgcagccagaccccaagtgcaactagtccaatcaggtggaggcgtcgtgcaacctggagggtccctccgcgttagctgcgccgcatcaggcgttaccttgtcagactacggcatgcattgggttaggcaagcccccggcaaggggctcgaatggatggctttcattcggaatgacgggagcgataaatattacgcggattcagttaaagggcggttcaccatcagccgcgacaatagcaaaaagacggtctccttacagatgtccagcttgcgggccgaagacacggctgtatactattgtgctaaaaatggcgagagcggccccctggattactggtactttgacctgtggggcagaggcaccctggtcacggtgtcctctggaggaggaggctccggaggaggaggctctggcggcggcggcagcgacattgtaatgacccagtcaccctccttccttagtgcctcagtcggagaccgcgtgactatcacttgtcgtgcctcacacggaattaataactacctcgcttggtatcagcaaaaaccaggcaaggccccgaaactattgatctacgccgcatctactctgcagagcggagtaccgagcagatttagtggttccggcagcggaaccgagttcaccctaaccatttccagtttgcagccagaggatttcgcgacctattactgccagcaatacgattcatacccgccaactttcggaagaggtaccaaggttgaaatcaagactacgaccccagcacctagacctcccaccccagctccaactatagcttcccagccattgtctctccggccagaggcgtgtcgaccagccgctggaggggccgttcatacaagaggactcgatttcgcttgcgatatctacatatgggcccctcttgccgggacatgcggtgtcctgcttctaagcttggttattaccctctattgctgcagcagggccgcccgcggcaccatcggcgccaggcgcacaggccagcctctgaaggaggacccttccgccgtgccagtgttctctgtggactacggcgagctggattttcagtggcgggagaaaaccccagagccacctgtgccctgcgtgcctgagcagaccgagtatgccacaatcgtgtttccatccggaatgggcacaagctcccctgcaaggagaggcagcgccgacggaccacggtccgcccagccactgcggcccgaggatggccactgttcttggcccctgcggagaaagcgtggatccggggaaggccgaggctcccttctaacatgtggagatgtcgaggaaaaccctggccctatggcgctgccagtcactgcattgttattgcctctggccctgcttctccatgcggcgcgcccagaagtgcagctggtcgagagcggaggcggactggttcaacccggaggcagcttgagactgtcctgcgcggccagcggcttcaacatcaaggatacctatatccactgggtgaggcaggctccaggaaagggcctggagtgggtggcaaggatttaccctactaatggatatacacgctacgctgattccgtgaagggacgctttacaatctcagcagatacatccaaaaacacggcctatttacagatgaatagtttgcgggccgaagacacggctgtatactattgttctcggtgggggggcgatggattttatgcgatggattactggggccagggcaccctggtaaccgtgtcaagcggctcaacatccgggtccggtaagccgggctccggcgaggggtctacaaagggagatatacagatgacacagtcccccagttccctgtccgcctcagtgggagaccgagtgacgattacctgtcgtgccagccaggacgtcaataccgccgtcgcttggtatcagcaaaaaccaggcaaggccccgaaactattgatctacagtgcctcttttctgtactccggggtgccgagcagatttagtggctccaggagcggaaccgatttcaccctaaccatttccagtttgcagccagaggatttcgcgacctattactgccagcaacactacaccacaccgccaactttcggacaaggaaccaaggttgaaatcaaaattgaagttatgtatcctcctccttacctagacaatgagaagagcaatggaaccattatccatgtgaaagggaaacacctttgtccaagtcccctatttcccgggccttcgaagcccttttgggtgctggtggtggttggtggagtcctggcttgctatagcttgctagtaacagtagcgtttattattttctgggtgaaacgcggccgcaagaaactgctctacatctttaaacagccgttcatgaggcctgtgcagacaacgcaggaagaggatggctgtagttgtcggtttccggaagaggaagaggggggctgcgagttgcgtgtcaaattttctcggtctgccgacgcccccgcgtaccagcaagggcagaaccagctttataatgagctgaatcttggacgacgggaggaatatgacgtgcttgacaagaggcgaggtagggaccctgagatggggggaaaacctcggaggaaaaacccacaggaaggcctgtataacgaactgcagaaggacaagatggctgaagcctactctgagattggaatgaaaggggaacgcagacgcggcaagggccatgatggcctctaccaaggtctaagcactgccaccaaggacacctatgacgcactccacatgcaagctctacctccccgttgataamc

SEQ ID NO: 12 - protein, 943 aa, synthetic construct
MALPVTALLLPLALLLHAARPQVQLVQSGGGVVQPGGSLRVSCAASGVTLSDYGMHWVRQAPGKGLEWMAFIRNDGSDKYYADSVKGRFTISRDNSKKTVSLQMSSLRAEDTAVYYCAKNGESGPLDYWYFDLWGRGTLVTVSSGGGGSGGGGSGGGGSDIVMTQSPSFLSASVGDRVTITCRASHGINNYLAWYQQKPGKAPKLLIYAASTLQSGVPSRFSGSGSGTEFTLTISSLQPEDFATYYCQQYDSYPPTFGRGTKVEIKTTTPAPRPPTPAPTIASQPLSLRPEACRPAAGGAVHTRGLDFACDIYIWAPLAGTCGVLLLSLVITLYCCSRAARGTIGARRTGQPLKEDPSAVPVFSVDYGELDFQWREKTPEPPVPCVPEQTEYATIVFPSGMGTSSPARRGSADGPRSAQPLRPEDGHCSWPLRRKRGSGEGRGSLLTCGDVEENPGPMALPVTALLLPLALLLHAARPEVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSGSTSGSGKPGSGEGSTKGDIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKIEVMYPPPYLDNEKSNGTIIHVKGKHLCPSPLFPGPSKPFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 13 - DNA, 2829 bp, synthetic construct
  source 1..2829: mol_type = other DNA
atggcactgccagtgaccgccctgctgctgcctctggccctgctgctgcacgcagccagaccccaggtgcagctgcagcagtctggacctgagctggtgaagccaggagcctccgtgaagatgtcttgcaaggccagcggctacaccttcacatcttatcacatccagtgggtgaagcagcggcccggacagggcctggagtggatcggatggatctacccaggcgacggctccacacagtataacgagaagttcaagggcaagaccacactgaccgccgataagagcagcagcaccgcctacatgctgctgagcagcctgaccagcgaggacagcgccatctacttttgcgccagggagggcacatactatgctatggactattggggccagggcaccagcgtgacagtgtctagcggaggaggaggctccggaggaggaggctctggcggcggcggcagcgacgtgctgatgacccagacaccactgagcctgcccgtgagcctgggcgatcaggtgagcatctcctgtagatcctctcagagcatcgtgcactccaacggcaatacctacctggagtggtatctgcagaagccaggccagtcccccaagctgctgatctataaggtgtctaatcggttcagcggcgtgcctgacagattttctggcagcggctccggcaccgacttcaccctgaagatcagccgggtggaggcagaggatctgggcgtgtactattgtttccagggctcccacgtgccacgcacctttggcggcggtaccaagctggagatcaagactacgaccccagcacctagacctcccaccccagctccaactatagcttcccagccattgtctctccggccagaggcgtgtcgaccagccgctggaggggccgttcatacaagaggactcgatttcgcttgcgatatctacatatgggcccctcttgccgggacatgcggtgtcctgcttctaagcttggttattaccctctattgctgcagcagggccgcccgcggcaccatcggcgccaggcgcacaggccagcctctgaaggaggacccttccgccgtgccagtgttctctgtggactacggcgagctggattttcagtggcgggagaaaaccccagagccacctgtgccctgcgtgcctgagcagaccgagtatgccacaatcgtgtttccatccggaatgggcacaagctcccctgcaaggagaggcagcgccgacggaccacggtccgcccagccactgcggcccgaggatggccactgttcttggcccctgcggagaaagcgtggatccggggaaggccgaggctcccttctaacatgtggagatgtcgaggaaaaccctggccctatggcgctgccagtcactgcattgttattgcctctggccctgcttctccatgcggcgcgcccacaagtgcagctgaaacagagcggaccaggactggttcaacccagccagagcttgagcatcacgtgcacggttagcggcttcagtctgaccaattatggtgtgcactgggtgaggcagtctccaggaaagggcctggagtggcttggagtcatttggagcggtgggaatacagattacaatacaccttttacgtcacgtctctccattaacaaggacaactccaaatcccaagtatttttcaaaatgaatagcctgcagagtaatgataccgccatctattactgtgcacgagctttgacatattacgactatgaatttgcctattggggtcaaggcacgctggtgaccgtatcaggctcaacatccgggtccggtaagccgggctccggcgaggggtctacaaagggagacatccttctgacacagagccccgtgatcctgtccgtgtcccccggcgagagagtatcattttcctgtagggcttctcagagcatcggaacaaatatccactggtatcagcaacggactaacggatcacctcgcctgctcataaagtacgccagtgaatctattagtggcataccgagccgcttcagcgggagtggctccggcacagactttactctgagtataaattccgtggaatctgaggacatcgcggactattactgccagcaaaacaataactggcccaccacgttcggcgcgggaactaaactagaactaaagattgaagttatgtatcctcctccttacctagacaatgagaagagcaatggaaccattatccatgtgaaagggaaacacctttgtccaagtcccctatttcccgggccttcgaagcccttttgggtgctggtggtggttggtggagtcctggcttgctatagcttgctagtaacagtagcgtttattattttctgggtgaaacgcggccgcaagaaactgctctacatctttaaacagccgttcatgaggcctgtgcagacaacgcaggaagaggatggctgtagttgtcggtttccggaagaggaagaggggggctgcgagttgcgtgtcaaattttctcggtctgccgacgcccccgcgtaccagcaagggcagaaccagctttataatgagctgaatcttggacgacgggaggaatatgacgtgcttgacaagaggcgaggtagggaccctgagatggggggaaaacctcggaggaaaaacccacaggaaggcctgtataacgaactgcagaaggacaagatggctgaagcctactctgagattggaatgaaaggggaacgcagacgcggcaagggccatgatggcctctaccaaggtctaagcactgccaccaaggacacctatgacgcactccacatgcaagctctacctccccgttgataa

SEQ ID NO: 14 - protein, 941 aa, synthetic construct
MALPVTALLLPLALLLHAARPQVQLQQSGPELVKPGASVKMSCKASGYTFTSYHIQWVKQRPGQGLEWIGWIYPGDGSTQYNEKFKGKTTLTADKSSSTAYMLLSSLTSEDSAIYFCAREGTYYAMDYWGQGTSVTVSSGGGGSGGGGSGGGGSDVLMTQTPLSLPVSLGDQVSISCRSSQSIVHSNGNTYLEWYLQKPGQSPKLLIYKVSNRFSGVPDRFSGSGSGTDFTLKISRVEAEDLGVYYCFQGSHVPRTFGGGTKLEIKTTTPAPRPPTPAPTIASQPLSLRPEACRPAAGGAVHTRGLDFACDIYIWAPLAGTCGVLLLSLVITLYCCSRAARGTIGARRTGQPLKEDPSAVPVFSVDYGELDFQWREKTPEPPVPCVPEQTEYATIVFPSGMGTSSPARRGSADGPRSAQPLRPEDGHCSWPLRRKRGSGEGRGSLLTCGDVEENPGPMALPVTALLLPLALLLHAARPQVQLKQSGPGLVQPSQSLSITCTVSGFSLTNYGVHWVRQSPGKGLEWLGVIWSGGNTDYNTPFTSRLSINKDNSKSQVFFKMNSLQSNDTAIYYCARALTYYDYEFAYWGQGTLVTVSGSTSGSGKPGSGEGSTKGDILLTQSPVILSVSPGERVSFSCRASQSIGTNIHWYQQRTNGSPRLLIKYASESISGIPSRFSGSGSGTDFTLSINSVESEDIADYYCQQNNNWPTTFGAGTKLELKIEVMYPPPYLDNEKSNGTIIHVKGKHLCPSPLFPGPSKPFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 15 - DNA, 2829 bp, synthetic construct
  source 1..2829: mol_type = other DNA
atggcactgccagtgaccgccctgctgctgcctctggccctgctgctgcacgcagccagaccccaagtgcaactagtccaatcaggtggaggcgtcgtgcaacctggagggtccctccgcgttagctgcgccgcatcaggcgttaccttgtcagactacggcatgcattgggttaggcaagcccccggcaaggggctcgaatggatggctttcattcggaatgacgggagcgataaatattacgcggattcagttaaagggcggttcaccatcagccgcgacaatagcaaaaagacggtctccttacagatgtccagcttgcgggccgaagacacggctgtatactattgtgctaaaaatggcgagagcggccccctggattactggtactttgacctgtggggcagaggcaccctggtcacggtgtcctctggaggaggaggctccggaggaggaggctctggcggcggcggcagcgacattgtaatgacccagtcaccctccttccttagtgcctcagtcggagaccgcgtgactatcacttgtcgtgcctcacacggaattaataactacctcgcttggtatcagcaaaaaccaggcaaggccccgaaactattgatctacgccgcatctactctgcagagcggagtaccgagcagatttagtggttccggcagcggaaccgagttcaccctaaccatttccagtttgcagccagaggatttcgcgacctattactgccagcaatacgattcatacccgccaactttcggaagaggtaccaaggttgaaatcaagactacgaccccagcacctagacctcccaccccagctccaactatagcttcccagccattgtctctccggccagaggcgtgtcgaccagccgctggaggggccgttcatacaagaggactcgatttcgcttgcgatatctacatatgggcccctcttgccgggacatgcggtgtcctgcttctaagcttggttattaccctctattgctgcagcagggccgcccgcggcaccatcggcgccaggcgcacaggccagcctctgaaggaggacccttccgccgtgccagtgttctctgtggactacggcgagctggattttcagtggcgggagaaaaccccagagccacctgtgccctgcgtgcctgagcagaccgagtatgccacaatcgtgtttccatccggaatgggcacaagctcccctgcaaggagaggcagcgccgacggaccacggtccgcccagccactgcggcccgaggatggccactgttcttggcccctgcggagaaagcgtggatccggggaaggccgaggctcccttctaacatgtggagatgtcgaggaaaaccctggccctatggcgctgccagtcactgcattgttattgcctctggccctgcttctccatgcggcgcgcccacaagtgcagctgaaacagagcggaccaggactggttcaacccagccagagcttgagcatcacgtgcacggttagcggcttcagtctgaccaattatggtgtgcactgggtgaggcagtctccaggaaagggcctggagtggcttggagtcatttggagcggtgggaatacagattacaatacaccttttacgtcacgtctctccattaacaaggacaactccaaatcccaagtatttttcaaaatgaatagcctgcagagtaatgataccgccatctattactgtgcacgagctttgacatattacgactatgaatttgcctattggggtcaaggcacgctggtgaccgtatcaggctcaacatccgggtccggtaagccgggctccggcgaggggtctacaaagggagacatccttctgacacagagccccgtgatcctgtccgtgtcccccggcgagagagtatcattttcctgtagggcttctcagagcatcggaacaaatatccactggtatcagcaacggactaacggatcacctcgcctgctcataaagtacgccagtgaatctattagtggcataccgagccgcttcagcgggagtggctccggcacagactttactctgagtataaattccgtggaatctgaggacatcgcggactattactgccagcaaaacaataactggcccaccacgttcggcgcgggaactaaactagaactaaagattgaagttatgtatcctcctccttacctagacaatgagaagagcaatggaaccattatccatgtgaaagggaaacacctttgtccaagtcccctatttcccgggccttcgaagcccttttgggtgctggtggtggttggtggagtcctggcttgctatagcttgctagtaacagtagcgtttattattttctgggtgaaacgcggccgcaagaaactgctctacatctttaaacagccgttcatgaggcctgtgcagacaacgcaggaagaggatggctgtagttgtcggtttccggaagaggaagaggggggctgcgagttgcgtgtcaaattttctcggtctgccgacgcccccgcgtaccagcaagggcagaaccagctttataatgagctgaatcttggacgacgggaggaatatgacgtgcttgacaagaggcgaggtagggaccctgagatggggggaaaacctcggaggaaaaacccacaggaaggcctgtataacgaactgcagaaggacaagatggctgaagcctactctgagattggaatgaaaggggaacgcagacgcggcaagggccatgatggcctctaccaaggtctaagcactgccaccaaggacacctatgacgcactccacatgcaagctctacctccccgttgataa

SEQ ID NO: 16 - protein, 941 aa, synthetic construct
MALPVTALLLPLALLLHAARPQVQLVQSGGGVVQPGGSLRVSCAASGVTLSDYGMHWVRQAPGKGLEWMAFIRNDGSDKYYADSVKGRFTISRDNSKKTVSLQMSSLRAEDTAVYYCAKNGESGPLDYWYFDLWGRGTLVTVSSGGGGSGGGGSGGGGSDIVMTQSPSFLSASVGDRVTITCRASHGINNYLAWYQQKPGKAPKLLIYAASTLQSGVPSRFSGSGSGTEFTLTISSLQPEDFATYYCQQYDSYPPTFGRGTKVEIKTTTPAPRPPTPAPTIASQPLSLRPEACRPAAGGAVHTRGLDFACDIYIWAPLAGTCGVLLLSLVITLYCCSRAARGTIGARRTGQPLKEDPSAVPVFSVDYGELDFQWREKTPEPPVPCVPEQTEYATIVFPSGMGTSSPARRGSADGPRSAQPLRPEDGHCSWPLRRKRGSGEGRGSLLTCGDVEENPGPMALPVTALLLPLALLLHAARPQVQLKQSGPGLVQPSQSLSITCTVSGFSLTNYGVHWVRQSPGKGLEWLGVIWSGGNTDYNTPFTSRLSINKDNSKSQVFFKMNSLQSNDTAIYYCARALTYYDYEFAYWGQGTLVTVSGSTSGSGKPGSGEGSTKGDILLTQSPVILSVSPGERVSFSCRASQSIGTNIHWYQQRTNGSPRLLIKYASESISGIPSRFSGSGSGTDFTLSINSVESEDIADYYCQQNNNWPTTFGAGTKLELKIEVMYPPPYLDNEKSNGTIIHVKGKHLCPSPLFPGPSKPFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 17 - DNA, 2544 bp, synthetic construct
  source 1..2544: mol_type = other DNA
atggcactgccagtgaccgccctgctgctgcctctggccctgctgctgcacgcagccagaccccaggtgcagctgcagcagtctggacctgagctggtgaagccaggagcctccgtgaagatgtcttgcaaggccagcggctacaccttcacatcttatcacatccagtgggtgaagcagcggcccggacagggcctggagtggatcggatggatctacccaggcgacggctccacacagtataacgagaagttcaagggcaagaccacactgaccgccgataagagcagcagcaccgcctacatgctgctgagcagcctgaccagcgaggacagcgccatctacttttgcgccagggagggcacatactatgctatggactattggggccagggcaccagcgtgacagtgtctagcggaggaggaggctccggaggaggaggctctggcggcggcggcagcgacgtgctgatgacccagacaccactgagcctgcccgtgagcctgggcgatcaggtgagcatctcctgtagatcctctcagagcatcgtgcactccaacggcaatacctacctggagtggtatctgcagaagccaggccagtcccccaagctgctgatctataaggtgtctaatcggttcagcggcgtgcctgacagattttctggcagcggctccggcaccgacttcaccctgaagatcagccgggtggaggcagaggatctgggcgtgtactattgtttccagggctcccacgtgccacgcacctttggcggcggtaccaagctggagatcaagattgaagttatgtatcctcctccttacctagacaatgagaagagcaatggaaccattatccatgtgaaagggaaacacctttgtccaagtcccctatttcccgggccttcgaagcccttttgggtgctggtggtggttggtggagtcctggcttgctatagcttgctagtaacagtagcgtttattattttctgggtgcggagaaagcgtggatccggggaaggccgaggctcccttctaacatgtggagatgtcgaggaaaaccctggccctatggcgctgccagtcactgcattgttattgcctctggccctgcttctccatgcggcgcgcccagaagtgcagctggtcgagagcggaggcggactggttcaacccggaggcagcttgagactgtcctgcgcggccagcggcttcaacatcaaggatacctatatccactgggtgaggcaggctccaggaaagggcctggagtgggtggcaaggatttaccctactaatggatatacacgctacgctgattccgtgaagggacgctttacaatctcagcagatacatccaaaaacacggcctatttacagatgaatagtttgcgggccgaagacacggctgtatactattgttctcggtgggggggcgatggattttatgcgatggattactggggccagggcaccctggtaaccgtgtcaagcggctcaacatccgggtccggtaagccgggctccggcgaggggtctacaaagggagatatacagatgacacagtcccccagttccctgtccgcctcagtgggagaccgagtgacgattacctgtcgtgccagccaggacgtcaataccgccgtcgcttggtatcagcaaaaaccaggcaaggccccgaaactattgatctacagtgcctcttttctgtactccggggtgccgagcagatttagtggctccaggagcggaaccgatttcaccctaaccatttccagtttgcagccagaggatttcgcgacctattactgccagcaacactacaccacaccgccaactttcggacaaggaaccaaggttgaaatcaaaactacgaccccagcacctagacctcccaccccagctccaactatagcttcccagccattgtctctccggccagaggcgtgtcgaccagccgctggaggggccgttcatacaagaggactcgatttcgcttgcgatatctacatatgggcccctcttgccgggacatgcggtgtcctgcttctaagcttggttattaccctctattgcaaacgcggccgcaagaaactgctctacatctttaaacagccgttcatgaggcctgtgcagacaacgcaggaagaggatggctgtagttgtcggtttccggaagaggaagaggggggctgcgagttgcgtgtcaaattttctcggtctgccgacgcccccgcgtaccagcaagggcagaaccagctttataatgagctgaatcttggacgacgggaggaatatgacgtgcttgacaagaggcgaggtagggaccctgagatggggggaaaacctcggaggaaaaacccacaggaaggcctgtataacgaactgcagaaggacaagatggctgaagcctactctgagattggaatgaaaggggaacgcagacgcggcaagggccatgatggcctctaccaaggtctaagcactgccaccaaggacacctatgacgcactccacatgcaagctctacctccccgttgataa

SEQ ID NO: 18 - protein, 846 aa, synthetic construct
MALPVTALLLPLALLLHAARPQVQLQQSGPELVKPGASVKMSCKASGYTFTSYHIQWVKQRPGQGLEWIGWIYPGDGSTQYNEKFKGKTTLTADKSSSTAYMLLSSLTSEDSAIYFCAREGTYYAMDYWGQGTSVTVSSGGGGSGGGGSGGGGSDVLMTQTPLSLPVSLGDQVSISCRSSQSIVHSNGNTYLEWYLQKPGQSPKLLIYKVSNRFSGVPDRFSGSGSGTDFTLKISRVEAEDLGVYYCFQGSHVPRTFGGGTKLEIKIEVMYPPPYLDNEKSNGTIIHVKGKHLCPSPLFPGPSKPFWVLVVVGGVLACYSLLVTVAFIIFWVRRKRGSGEGRGSLLTCGDVEENPGPMALPVTALLLPLALLLHAARPEVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSGSTSGSGKPGSGEGSTKGDIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 19 - DNA, 2544 bp, synthetic construct
  source 1..2544: mol_type = other DNA
atggcactgccagtgaccgccctgctgctgcctctggccctgctgctgcacgcagccagaccccaagtgcaactagtccaatcaggtggaggcgtcgtgcaacctggagggtccctccgcgttagctgcgccgcatcaggcgttaccttgtcagactacggcatgcattgggttaggcaagcccccggcaaggggctcgaatggatggctttcattcggaatgacgggagcgataaatattacgcggattcagttaaagggcggttcaccatcagccgcgacaatagcaaaaagacggtctccttacagatgtccagcttgcgggccgaagacacggctgtatactattgtgctaaaaatggcgagagcggccccctggattactggtactttgacctgtggggcagaggcaccctggtcacggtgtcctctggaggaggaggctccggaggaggaggctctggcggcggcggcagcgacattgtaatgacccagtcaccctccttccttagtgcctcagtcggagaccgcgtgactatcacttgtcgtgcctcacacggaattaataactacctcgcttggtatcagcaaaaaccaggcaaggccccgaaactattgatctacgccgcatctactctgcagagcggagtaccgagcagatttagtggttccggcagcggaaccgagttcaccctaaccatttccagtttgcagccagaggatttcgcgacctattactgccagcaatacgattcatacccgccaactttcggaagaggtaccaaggttgaaatcaagattgaagttatgtatcctcctccttacctagacaatgagaagagcaatggaaccattatccatgtgaaagggaaacacctttgtccaagtcccctatttcccgggccttcgaagcccttttgggtgctggtggtggttggtggagtcctggcttgctatagcttgctagtaacagtagcgtttattattttctgggtgcggagaaagcgtggatccggggaaggccgaggctcccttctaacatgtggagatgtcgaggaaaaccctggccctatggcgctgccagtcactgcattgttattgcctctggccctgcttctccatgcggcgcgcccagaagtgcagctggtcgagagcggaggcggactggttcaacccggaggcagcttgagactgtcctgcgcggccagcggcttcaacatcaaggatacctatatccactgggtgaggcaggctccaggaaagggcctggagtgggtggcaaggatttaccctactaatggatatacacgctacgctgattccgtgaagggacgctttacaatctcagcagatacatccaaaaacacggcctatttacagatgaatagtttgcgggccgaagacacggctgtatactattgttctcggtgggggggcgatggattttatgcgatggattactggggccagggcaccctggtaaccgtgtcaagcggctcaacatccgggtccggtaagccgggctccggcgaggggtctacaaagggagatatacagatgacacagtcccccagttccctgtccgcctcagtgggagaccgagtgacgattacctgtcgtgccagccaggacgtcaataccgccgtcgcttggtatcagcaaaaaccaggcaaggccccgaaactattgatctacagtgcctcttttctgtactccggggtgccgagcagatttagtggctccaggagcggaaccgatttcaccctaaccatttccagtttgcagccagaggatttcgcgacctattactgccagcaacactacaccacaccgccaactttcggacaaggaaccaaggttgaaatcaaaactacgaccccagcacctagacctcccaccccagctccaactatagcttcccagccattgtctctccggccagaggcgtgtcgaccagccgctggaggggccgttcatacaagaggactcgatttcgcttgcgatatctacatatgggcccctcttgccgggacatgcggtgtcctgcttctaagcttggttattaccctctattgcaaacgcggccgcaagaaactgctctacatctttaaacagccgttcatgaggcctgtgcagacaacgcaggaagaggatggctgtagttgtcggtttccggaagaggaagaggggggctgcgagttgcgtgtcaaattttctcggtctgccgacgcccccgcgtaccagcaagggcagaaccagctttataatgagctgaatcttggacgacgggaggaatatgacgtgcttgacaagaggcgaggtagggaccctgagatggggggaaaacctcggaggaaaaacccacaggaaggcctgtataacgaactgcagaaggacaagatggctgaagcctactctgagattggaatgaaaggggaacgcagacgcggcaagggccatgatggcctctaccaaggtctaagcactgccaccaaggacacctatgacgcactccacatgcaagctctacctccccgttgataa

SEQ ID NO: 20 - protein, 846 aa, synthetic construct
MALPVTALLLPLALLLHAARPQVQLVQSGGGVVQPGGSLRVSCAASGVTLSDYGMHWVRQAPGKGLEWMAFIRNDGSDKYYADSVKGRFTISRDNSKKTVSLQMSSLRAEDTAVYYCAKNGESGPLDYWYFDLWGRGTLVTVSSGGGGSGGGGSGGGGSDIVMTQSPSFLSASVGDRVTITCRASHGINNYLAWYQQKPGKAPKLLIYAASTLQSGVPSRFSGSGSGTEFTLTISSLQPEDFATYYCQQYDSYPPTFGRGTKVEIKIEVMYPPPYLDNEKSNGTIIHVKGKHLCPSPLFPGPSKPFWVLVVVGGVLACYSLLVTVAFIIFWVRRKRGSGEGRGSLLTCGDVEENPGPMALPVTALLLPLALLLHAARPEVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSGSTSGSGKPGSGEGSTKGDIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 21 - DNA, 3048 bp, synthetic construct
  source 1..3048: mol_type = other DNA
atggcactgccagtgaccgccctgctgctgcctctggccctgctgctgcacgcagccagaccccaagtgcaactagtccaatcaggtggaggcgtcgtgcaacctggagggtccctccgcgttagctgcgccgcatcaggcgttaccttgtcagactacggcatgcattgggttaggcaagcccccggcaaggggctcgaatggatggctttcattcggaatgacgggagcgataaatattacgcggattcagttaaagggcggttcaccatcagccgcgacaatagcaaaaagacggtctccttacagatgtccagcttgcgggccgaagacacggctgtatactattgtgctaaaaatggcgagagcggccccctggattactggtactttgacctgtggggcagaggcaccctggtcacggtgtcctctggaggaggaggctccggaggaggaggctctggcggcggcggcagcgacattgtaatgacccagtcaccctccttccttagtgcctcagtcggagaccgcgtgactatcacttgtcgtgcctcacacggaattaataactacctcgcttggtatcagcaaaaaccaggcaaggccccgaaactattgatctacgccgcatctactctgcagagcggagtaccgagcagatttagtggttccggcagcggaaccgagttcaccctaaccatttccagtttgcagccagaggatttcgcgacctattactgccagcaatacgattcatacccgccaactttcggaagaggtaccaaggttgaaatcaagattgaagttatgtatcctcctccttacctagacaatgagaagagcaatggaaccattatccatgtgaaagggaaacacctttgtccaagtcccctatttcccgggccttcgaagcccttttgggtgctggtggtggttggtggagtcctggcttgctatagcttgctagtaacagtagcgtttattattttctgggtgctgcgccacaggagacagggcaagcactggaccagcacccagcggaaggccgactttcagcaccctgccggcgccgtgggccctgagcctaccgacaggggcctgcagtggaggagctccccagccgccgatgcccaggaggagaatctgtacgccgccgtgaagcacacccagccagaggacggcgtggagatggacacccgctccccacacgacgaggatccacaggccgtgacctacgccgaggtgaagcacagccgccccagacgcgagatggccagcccacccagccccctgtccggcgagttcctggacaccaaggacaggcaggccgaggaggaccggcagatggacaccgaggccgccgcctccgaggccccccaggacgtgacctacgcccagctgcactccctgaccctgcggagagaggccaccgagcccccacccagccaggagggcccctcccccgccgtgcctagcatctacgccaccctggccatccaccggagaaagcgtggatccggggaaggccgaggctcccttctaacatgtggagatgtcgaggaaaaccctggccctatggcgctgccagtcactgcattgttattgcctctggccctgcttctccatgcggcgcgcccagaagtgcagctggtcgagagcggaggcggactggttcaacccggaggcagcttgagactgtcctgcgcggccagcggcttcaacatcaaggatacctatatccactgggtgaggcaggctccaggaaagggcctggagtgggtggcaaggatttaccctactaatggatatacacgctacgctgattccgtgaagggacgctttacaatctcagcagatacatccaaaaacacggcctatttacagatgaatagtttgcgggccgaagacacggctgtatactattgttctcggtgggggggcgatggattttatgcgatggattactggggccagggcaccctggtaaccgtgtcaagcggctcaacatccgggtccggtaagccgggctccggcgaggggtctacaaagggagatatacagatgacacagtcccccagttccctgtccgcctcagtgggagaccgagtgacgattacctgtcgtgccagccaggacgtcaataccgccgtcgcttggtatcagcaaaaaccaggcaaggccccgaaactattgatctacagtgcctcttttctgtactccggggtgccgagcagatttagtggctccaggagcggaaccgatttcaccctaaccatttccagtttgcagccagaggatttcgcgacctattactgccagcaacactacaccacaccgccaactttcggacaaggaaccaaggttgaaatcaaaactacgaccccagcacctagacctcccaccccagctccaactatagcttcccagccattgtctctccggccagaggcgtgtcgaccagccgctggaggggccgttcatacaagaggactcgatttcgcttgcgatatctacatatgggcccctcttgccgggacatgcggtgtcctgcttctaagcttggttattaccctctattgcaaacgcggccgcaagaaactgctctacatctttaaacagccgttcatgaggcctgtgcagacaacgcaggaagaggatggctgtagttgtcggtttccggaagaggaagaggggggctgcgagttgcgtgtcaaattttctcggtctgccgacgcccccgcgtaccagcaagggcagaaccagctttataatgagctgaatcttggacgacgggaggaatatgacgtgcttgacaagaggcgaggtagggaccctgagatggggggaaaacctcggaggaaaaacccacaggaaggcctgtataacgaactgcagaaggacaagatggctgaagcctactctgagattggaatgaaaggggaacgcagacgcggcaagggccatgatggcctctaccaaggtctaagcactgccaccaaggacacctatgacgcactccacatgcaagctctacctccccgttgataa

SEQ ID NO: 22 - protein, 1014 aa, synthetic construct
MALPVTALLLPLALLLHAARPQVQLVQSGGGVVQPGGSLRVSCAASGVTLSDYGMHWVRQAPGKGLEWMAFIRNDGSDKYYADSVKGRFTISRDNSKKTVSLQMSSLRAEDTAVYYCAKNGESGPLDYWYFDLWGRGTLVTVSSGGGGSGGGGSGGGGSDIVMTQSPSFLSASVGDRVTITCRASHGINNYLAWYQQKPGKAPKLLIYAASTLQSGVPSRFSGSGSGTEFTLTISSLQPEDFATYYCQQYDSYPPTFGRGTKVEIKIEVMYPPPYLDNEKSNGTIIHVKGKHLCPSPLFPGPSKPFWVLVVVGGVLACYSLLVTVAFIIFWVLRHRRQGKHWTSTQRKADFQHPAGAVGPEPTDRGLQWRSSPAADAQEENLYAAVKHTQPEDGVEMDTRSPHDEDPQAVTYAEVKHSRPRREMASPPSPLSGEFLDTKDRQAEEDRQMDTEAAASEAPQDVTYAQLHSLTLRREATEPPPSQEGPSPAVPSIYATLAIHRRKRGSGEGRGSLLTCGDVEENPGPMALPVTALLLPLALLLHAARPEVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSGSTSGSGKPGSGEGSTKGDIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 23 - DNA, 2796 bp, synthetic construct
  source 1..2796: mol_type = other DNA
atggcactgccagtgaccgccctgctgctgcctctggccctgctgctgcacgcagccagaccccaagtgcaactagtccaatcaggtggaggcgtcgtgcaacctggagggtccctccgcgttagctgcgccgcatcaggcgttaccttgtcagactacggcatgcattgggttaggcaagcccccggcaaggggctcgaatggatggctttcattcggaatgacgggagcgataaatattacgcggattcagttaaagggcggttcaccatcagccgcgacaatagcaaaaagacggtctccttacagatgtccagcttgcgggccgaagacacggctgtatactattgtgctaaaaatggcgagagcggccccctggattactggtactttgacctgtggggcagaggcaccctggtcacggtgtcctctggaggaggaggctccggaggaggaggctctggcggcggcggcagcgacattgtaatgacccagtcaccctccttccttagtgcctcagtcggagaccgcgtgactatcacttgtcgtgcctcacacggaattaataactacctcgcttggtatcagcaaaaaccaggcaaggccccgaaactattgatctacgccgcatctactctgcagagcggagtaccgagcagatttagtggttccggcagcggaaccgagttcaccctaaccatttccagtttgcagccagaggatttcgcgacctattactgccagcaatacgattcatacccgccaactttcggaagaggtaccaaggttgaaatcaagattgaagttatgtatcctcctccttacctagacaatgagaagagcaatggaaccattatccatgtgaaagggaaacacctttgtccaagtcccctatttcccgggccttcgaagcccttttgggtgctggtggtggttggtggagtcctggcttgctatagcttgctagtaacagtagcgtttattattttctgggtgcataggtggtgctcaaacaaaaagaatgctgccgtcatggaccaggagagcgcgggcaatcggaccgcaaactcagaggactcagatgaacaagatccacaggaagtgacctacactcagctgaaccattgtgtgtttacacagcgcaagattactcgtccaagccagcgtcctaagacccccccgaccgatatcattgtgtataccgagcttcctaatgccgaatcccgcagcaaggtggtctcctgcccgcggagaaagcgtggatccggggaaggccgaggctcccttctaacatgtggagatgtcgaggaaaaccctggccctatggcgctgccagtcactgcattgttattgcctctggccctgcttctccatgcggcgcgcccagaagtgcagctggtcgagagcggaggcggactggttcaacccggaggcagcttgagactgtcctgcgcggccagcggcttcaacatcaaggatacctatatccactgggtgaggcaggctccaggaaagggcctggagtgggtggcaaggatttaccctactaatggatatacacgctacgctgattccgtgaagggacgctttacaatctcagcagatacatccaaaaacacggcctatttacagatgaatagtttgcgggccgaagacacggctgtatactattgttctcggtgggggggcgatggattttatgcgatggattactggggccagggcaccctggtaaccgtgtcaagcggctcaacatccgggtccggtaagccgggctccggcgaggggtctacaaagggagatatacagatgacacagtcccccagttccctgtccgcctcagtgggagaccgagtgacgattacctgtcgtgccagccaggacgtcaataccgccgtcgcttggtatcagcaaaaaccaggcaaggccccgaaactattgatctacagtgcctcttttctgtactccggggtgccgagcagatttagtggctccaggagcggaaccgatttcaccctaaccatttccagtttgcagccagaggatttcgcgacctattactgccagcaacactacaccacaccgccaactttcggacaaggaaccaaggttgaaatcaaaactacgaccccagcacctagacctcccaccccagctccaactatagcttcccagccattgtctctccggccagaggcgtgtcgaccagccgctggaggggccgttcatacaagaggactcgatttcgcttgcgatatctacatatgggcccctcttgccgggacatgcggtgtcctgcttctaagcttggttattaccctctattgcaaacgcggccgcaagaaactgctctacatctttaaacagccgttcatgaggcctgtgcagacaacgcaggaagaggatggctgtagttgtcggtttccggaagaggaagaggggggctgcgagttgcgtgtcaaattttctcggtctgccgacgcccccgcgtaccagcaagggcagaaccagctttataatgagctgaatcttggacgacgggaggaatatgacgtgcttgacaagaggcgaggtagggaccctgagatggggggaaaacctcggaggaaaaacccacaggaaggcctgtataacgaactgcagaaggacaagatggctgaagcctactctgagattggaatgaaaggggaacgcagacgcggcaagggccatgatggcctctaccaaggtctaagcactgccaccaaggacacctatgacgcactccacatgcaagctctacctccccgttgataa

SEQ ID NO: 24 - protein, 930 aa, synthetic construct
MALPVTALLLPLALLLHAARPQVQLVQSGGGVVQPGGSLRVSCAASGVTLSDYGMHWVRQAPGKGLEWMAFIRNDGSDKYYADSVKGRFTISRDNSKKTVSLQMSSLRAEDTAVYYCAKNGESGPLDYWYFDLWGRGTLVTVSSGGGGSGGGGSGGGGSDIVMTQSPSFLSASVGDRVTITCRASHGINNYLAWYQQKPGKAPKLLIYAASTLQSGVPSRFSGSGSGTEFTLTISSLQPEDFATYYCQQYDSYPPTFGRGTKVEIKIEVMYPPPYLDNEKSNGTIIHVKGKHLCPSPLFPGPSKPFWVLVVVGGVLACYSLLVTVAFIIFWVHRWCSNKKNAAVMDQESAGNRTANSEDSDEQDPQEVTYTQLNHCVFTQRKITRPSQRPKTPPTDIIVYTELPNAESRSKVVSCPRRKRGSGEGRGSLLTCGDVEENPGPMALPVTALLLPLALLLHAARPEVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSGSTSGSGKPGSGEGSTKGDIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 25 - DNA, 3048 bp, synthetic construct
  source 1..3048: mol_type = other DNA
atggcactgccagtgaccgccctgctgctgcctctggccctgctgctgcacgcagccagaccccaggtgcagctgcagcagtctggacctgagctggtgaagccaggagcctccgtgaagatgtcttgcaaggccagcggctacaccttcacatcttatcacatccagtgggtgaagcagcggcccggacagggcctggagtggatcggatggatctacccaggcgacggctccacacagtataacgagaagttcaagggcaagaccacactgaccgccgataagagcagcagcaccgcctacatgctgctgagcagcctgaccagcgaggacagcgccatctacttttgcgccagggagggcacatactatgctatggactattggggccagggcaccagcgtgacagtgtctagcggaggaggaggctccggaggaggaggctctggcggcggcggcagcgacgtgctgatgacccagacaccactgagcctgcccgtgagcctgggcgatcaggtgagcatctcctgtagatcctctcagagcatcgtgcactccaacggcaatacctacctggagtggtatctgcagaagccaggccagtcccccaagctgctgatctataaggtgtctaatcggttcagcggcgtgcctgacagattttctggcagcggctccggcaccgacttcaccctgaagatcagccgggtggaggcagaggatctgggcgtgtactattgtttccagggctcccacgtgccacgcacctttggcggcggtaccaagctggagatcaagattgaagttatgtatcctcctccttacctagacaatgagaagagcaatggaaccattatccatgtgaaagggaaacacctttgtccaagtcccctatttcccgggccttcgaagcccttttgggtgctggtggtggttggtggagtcctggcttgctatagcttgctagtaacagtagcgtttattattttctgggtgctgcgccacaggagacagggcaagcactggaccagcacccagcggaaggccgactttcagcaccctgccggcgccgtgggccctgagcctaccgacaggggcctgcagtggaggagctccccagccgccgatgcccaggaggagaatctgtacgccgccgtgaagcacacccagccagaggacggcgtggagatggacacccgctccccacacgacgaggatccacaggccgtgacctacgccgaggtgaagcacagccgccccagacgcgagatggccagcccacccagccccctgtccggcgagttcctggacaccaaggacaggcaggccgaggaggaccggcagatggacaccgaggccgccgcctccgaggccccccaggacgtgacctacgcccagctgcactccctgaccctgcggagagaggccaccgagcccccacccagccaggagggcccctcccccgccgtgcctagcatctacgccaccctggccatccaccggagaaagcgtggatccggggaaggccgaggctcccttctaacatgtggagatgtcgaggaaaaccctggccctatggcgctgccagtcactgcattgttattgcctctggccctgcttctccatgcggcgcgcccagaagtgcagctggtcgagagcggaggcggactggttcaacccggaggcagcttgagactgtcctgcgcggccagcggcttcaacatcaaggatacctatatccactgggtgaggcaggctccaggaaagggcctggagtgggtggcaaggatttaccctactaatggatatacacgctacgctgattccgtgaagggacgctttacaatctcagcagatacatccaaaaacacggcctatttacagatgaatagtttgcgggccgaagacacggctgtatactattgttctcggtgggggggcgatggattttatgcgatggattactggggccagggcaccctggtaaccgtgtcaagcggctcaacatccgggtccggtaagccgggctccggcgaggggtctacaaagggagatatacagatgacacagtcccccagttccctgtccgcctcagtgggagaccgagtgacgattacctgtcgtgccagccaggacgtcaataccgccgtcgcttggtatcagcaaaaaccaggcaaggccccgaaactattgatctacagtgcctcttttctgtactccggggtgccgagcagatttagtggctccaggagcggaaccgatttcaccctaaccatttccagtttgcagccagaggatttcgcgacctattactgccagcaacactacaccacaccgccaactttcggacaaggaaccaaggttgaaatcaaaactacgaccccagcacctagacctcccaccccagctccaactatagcttcccagccattgtctctccggccagaggcgtgtcgaccagccgctggaggggccgttcatacaagaggactcgatttcgcttgcgatatctacatatgggcccctcttgccgggacatgcggtgtcctgcttctaagcttggttattaccctctattgcaaacgcggccgcaagaaactgctctacatctttaaacagccgttcatgaggcctgtgcagacaacgcaggaagaggatggctgtagttgtcggtttccggaagaggaagaggggggctgcgagttgcgtgtcaaattttctcggtctgccgacgcccccgcgtaccagcaagggcagaaccagctttataatgagctgaatcttggacgacgggaggaatatgacgtgcttgacaagaggcgaggtagggaccctgagatggggggaaaacctcggaggaaaaacccacaggaaggcctgtataacgaactgcagaaggacaagatggctgaagcctactctgagattggaatgaaaggggaacgcagacgcggcaagggccatgatggcctctaccaaggtctaagcactgccaccaaggacacctatgacgcactccacatgcaagctctacctccccgttgataa

SEQ ID NO: 26 - protein, 1014 aa, synthetic construct
MALPVTALLLPLALLLHAARPQVQLQQSGPELVKPGASVKMSCKASGYTFTSYHIQWVKQRPGQGLEWIGWIYPGDGSTQYNEKFKGKTTLTADKSSSTAYMLLSSLTSEDSAIYFCAREGTYYAMDYWGQGTSVTVSSGGGGSGGGGSGGGGSDVLMTQTPLSLPVSLGDQVSISCRSSQSIVHSNGNTYLEWYLQKPGQSPKLLIYKVSNRFSGVPDRFSGSGSGTDFTLKISRVEAEDLGVYYCFQGSHVPRTFGGGTKLEIKIEVMYPPPYLDNEKSNGTIIHVKGKHLCPSPLFPGPSKPFWVLVVVGGVLACYSLLVTVAFIIFWVLRHRRQGKHWTSTQRKADFQHPAGAVGPEPTDRGLQWRSSPAADAQEENLYAAVKHTQPEDGVEMDTRSPHDEDPQAVTYAEVKHSRPRREMASPPSPLSGEFLDTKDRQAEEDRQMDTEAAASEAPQDVTYAQLHSLTLRREATEPPPSQEGPSPAVPSIYATLAIHRRKRGSGEGRGSLLTCGDVEENPGPMALPVTALLLPLALLLHAARPEVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSGSTSGSGKPGSGEGSTKGDIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 27 - DNA, 2796 bp, synthetic construct
  source 1..2796: mol_type = other DNA
atggcactgccagtgaccgccctgctgctgcctctggccctgctgctgcacgcagccagaccccaggtgcagctgcagcagtctggacctgagctggtgaagccaggagcctccgtgaagatgtcttgcaaggccagcggctacaccttcacatcttatcacatccagtgggtgaagcagcggcccggacagggcctggagtggatcggatggatctacccaggcgacggctccacacagtataacgagaagttcaagggcaagaccacactgaccgccgataagagcagcagcaccgcctacatgctgctgagcagcctgaccagcgaggacagcgccatctacttttgcgccagggagggcacatactatgctatggactattggggccagggcaccagcgtgacagtgtctagcggaggaggaggctccggaggaggaggctctggcggcggcggcagcgacgtgctgatgacccagacaccactgagcctgcccgtgagcctgggcgatcaggtgagcatctcctgtagatcctctcagagcatcgtgcactccaacggcaatacctacctggagtggtatctgcagaagccaggccagtcccccaagctgctgatctataaggtgtctaatcggttcagcggcgtgcctgacagattttctggcagcggctccggcaccgacttcaccctgaagatcagccgggtggaggcagaggatctgggcgtgtactattgtttccagggctcccacgtgccacgcacctttggcggcggtaccaagctggagatcaagattgaagttatgtatcctcctccttacctagacaatgagaagagcaatggaaccattatccatgtgaaagggaaacacctttgtccaagtcccctatttcccgggccttcgaagcccttttgggtgctggtggtggttggtggagtcctggcttgctatagcttgctagtaacagtagcgtttattattttctgggtgcataggtggtgctcaaacaaaaagaatgctgccgtcatggaccaggagagcgcgggcaatcggaccgcaaactcagaggactcagatgaacaagatccacaggaagtgacctacactcagctgaaccattgtgtgtttacacagcgcaagattactcgtccaagccagcgtcctaagacccccccgaccgatatcattgtgtataccgagcttcctaatgccgaatcccgcagcaaggtggtctcctgcccgcggagaaagcgtggatccggggaaggccgaggctcccttctaacatgtggagatgtcgaggaaaaccctggccctatggcgctgccagtcactgcattgttattgcctctggccctgcttctccatgcggcgcgcccagaagtgcagctggtcgagagcggaggcggactggttcaacccggaggcagcttgagactgtcctgcgcggccagcggcttcaacatcaaggatacctatatccactgggtgaggcaggctccaggaaagggcctggagtgggtggcaaggatttaccctactaatggatatacacgctacgctgattccgtgaagggacgctttacaatctcagcagatacatccaaaaacacggcctatttacagatgaatagtttgcgggccgaagacacggctgtatactattgttctcggtgggggggcgatggattttatgcgatggattactggggccagggcaccctggtaaccgtgtcaagcggctcaacatccgggtccggtaagccgggctccggcgaggggtctacaaagggagatatacagatgacacagtcccccagttccctgtccgcctcagtgggagaccgagtgacgattacctgtcgtgccagccaggacgtcaataccgccgtcgcttggtatcagcaaaaaccaggcaaggccccgaaactattgatctacagtgcctcttttctgtactccggggtgccgagcagatttagtggctccaggagcggaaccgatttcaccctaaccatttccagtttgcagccagaggatttcgcgacctattactgccagcaacactacaccacaccgccaactttcggacaaggaaccaaggttgaaatcaaaactacgaccccagcacctagacctcccaccccagctccaactatagcttcccagccattgtctctccggccagaggcgtgtcgaccagccgctggaggggccgttcatacaagaggactcgatttcgcttgcgatatctacatatgggcccctcttgccgggacatgcggtgtcctgcttctaagcttggttattaccctctattgcaaacgcggccgcaagaaactgctctacatctttaaacagccgttcatgaggcctgtgcagacaacgcaggaagaggatggctgtagttgtcggtttccggaagaggaagaggggggctgcgagttgcgtgtcaaattttctcggtctgccgacgcccccgcgtaccagcaagggcagaaccagctttataatgagctgaatcttggacgacgggaggaatatgacgtgcttgacaagaggcgaggtagggaccctgagatggggggaaaacctcggaggaaaaacccacaggaaggcctgtataacgaactgcagaaggacaagatggctgaagcctactctgagattggaatgaaaggggaacgcagacgcggcaagggccatgatggcctctaccaaggtctaagcactgccaccaaggacacctatgacgcactccacatgcaagctctacctccccgttgataa

SEQ ID NO: 28 - protein, 930 aa, synthetic construct
MALPVTALLLPLALLLHAARPQVQLQQSGPELVKPGASVKMSCKASGYTFTSYHIQWVKQRPGQGLEWIGWIYPGDGSTQYNEKFKGKTTLTADKSSSTAYMLLSSLTSEDSAIYFCAREGTYYAMDYWGQGTSVTVSSGGGGSGGGGSGGGGSDVLMTQTPLSLPVSLGDQVSISCRSSQSIVHSNGNTYLEWYLQKPGQSPKLLIYKVSNRFSGVPDRFSGSGSGTDFTLKISRVEAEDLGVYYCFQGSHVPRTFGGGTKLEIKIEVMYPPPYLDNEKSNGTIIHVKGKHLCPSPLFPGPSKPFWVLVVVGGVLACYSLLVTVAFIIFWVHRWCSNKKNAAVMDQESAGNRTANSEDSDEQDPQEVTYTQLNHCVFTQRKITRPSQRPKTPPTDIIVYTELPNAESRSKVVSCPRRKRGSGEGRGSLLTCGDVEENPGPMALPVTALLLPLALLLHAARPEVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSGSTSGSGKPGSGEGSTKGDIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 29 - DNA, 3048 bp, synthetic construct
  source 1..3048: mol_type = other DNA
atggcactgccagtgaccgccctgctgctgcctctggccctgctgctgcacgcagccagaccccaagtgcaactagtccaatcaggtggaggcgtcgtgcaacctggagggtccctccgcgttagctgcgccgcatcaggcgttaccttgtcagactacggcatgcattgggttaggcaagcccccggcaaggggctcgaatggatggctttcattcggaatgacgggagcgataaatattacgcggattcagttaaagggcggttcaccatcagccgcgacaatagcaaaaagacggtctccttacagatgtccagcttgcgggccgaagacacggctgtatactattgtgctaaaaatggcgagagcggccccctggattactggtactttgacctgtggggcagaggcaccctggtcacggtgtcctctggaggaggaggctccggaggaggaggctctggcggcggcggcagcgacattgtaatgacccagtcaccctccttccttagtgcctcagtcggagaccgcgtgactatcacttgtcgtgcctcacacggaattaataactacctcgcttggtatcagcaaaaaccaggcaaggccccgaaactattgatctacgccgcatctactctgcagagcggagtaccgagcagatttagtggttccggcagcggaaccgagttcaccctaaccatttccagtttgcagccagaggatttcgcgacctattactgccagcaatacgattcatacccgccaactttcggaagaggtaccaaggttgaaatcaagactacgaccccagcacctagacctcccaccccagctccaactatagcttcccagccattgtctctccggccagaggcgtgtcgaccagccgctggaggggccgttcatacaagaggactcgatttcgcttgcgatatctacatatgggcccctcttgccgggacatgcggtgtcctgcttctaagcttggttattaccctctattgcctgcgccacaggagacagggcaagcactggaccagcacccagcggaaggccgactttcagcaccctgccggcgccgtgggccctgagcctaccgacaggggcctgcagtggaggagctccccagccgccgatgcccaggaggagaatctgtacgccgccgtgaagcacacccagccagaggacggcgtggagatggacacccgctccccacacgacgaggatccacaggccgtgacctacgccgaggtgaagcacagccgccccagacgcgagatggccagcccacccagccccctgtccggcgagttcctggacaccaaggacaggcaggccgaggaggaccggcagatggacaccgaggccgccgcctccgaggccccccaggacgtgacctacgcccagctgcactccctgaccctgcggagagaggccaccgagcccccacccagccaggagggcccctcccccgccgtgcctagcatctacgccaccctggccatccaccggagaaagcgtggatccggggaaggccgaggctcccttctaacatgtggagatgtcgaggaaaaccctggccctatggcgctgccagtcactgcattgttattgcctctggccctgcttctccatgcggcgcgcccagaagtgcagctggtcgagagcggaggcggactggttcaacccggaggcagcttgagactgtcctgcgcggccagcggcttcaacatcaaggatacctatatccactgggtgaggcaggctccaggaaagggcctggagtgggtggcaaggatttaccctactaatggatatacacgctacgctgattccgtgaagggacgctttacaatctcagcagatacatccaaaaacacggcctatttacagatgaatagtttgcgggccgaagacacggctgtatactattgttctcggtgggggggcgatggattttatgcgatggattactggggccagggcaccctggtaaccgtgtcaagcggctcaacatccgggtccggtaagccgggctccggcgaggggtctacaaagggagatatacagatgacacagtcccccagttccctgtccgcctcagtgggagaccgagtgacgattacctgtcgtgccagccaggacgtcaataccgccgtcgcttggtatcagcaaaaaccaggcaaggccccgaaactattgatctacagtgcctcttttctgtactccggggtgccgagcagatttagtggctccaggagcggaaccgatttcaccctaaccatttccagtttgcagccagaggatttcgcgacctattactgccagcaacactacaccacaccgccaactttcggacaaggaaccaaggttgaaatcaaaattgaagttatgtatcctcctccttacctagacaatgagaagagcaatggaaccattatccatgtgaaagggaaacacctttgtccaagtcccctatttcccgggccttcgaagcccttttgggtgctggtggtggttggtggagtcctggcttgctatagcttgctagtaacagtagcgtttattattttctgggtgaaacgcggccgcaagaaactgctctacatctttaaacagccgttcatgaggcctgtgcagacaacgcaggaagaggatggctgtagttgtcggtttccggaagaggaagaggggggctgcgagttgcgtgtcaaattttctcggtctgccgacgcccccgcgtaccagcaagggcagaaccagctttataatgagctgaatcttggacgacgggaggaatatgacgtgcttgacaagaggcgaggtagggaccctgagatggggggaaaacctcggaggaaaaacccacaggaaggcctgtataacgaactgcagaaggacaagatggctgaagcctactctgagattggaatgaaaggggaacgcagacgcggcaagggccatgatggcctctaccaaggtctaagcactgccaccaaggacacctatgacgcactccacatgcaagctctacctccccgttgataa

SEQ ID NO: 30 - protein, 1014 aa, synthetic construct
MALPVTALLLPLALLLHAARPQVQLVQSGGGVVQPGGSLRVSCAASGVTLSDYGMHWVRQAPGKGLEWMAFIRNDGSDKYYADSVKGRFTISRDNSKKTVSLQMSSLRAEDTAVYYCAKNGESGPLDYWYFDLWGRGTLVTVSSGGGGSGGGGSGGGGSDIVMTQSPSFLSASVGDRVTITCRASHGINNYLAWYQQKPGKAPKLLIYAASTLQSGVPSRFSGSGSGTEFTLTISSLQPEDFATYYCQQYDSYPPTFGRGTKVEIKTTTPAPRPPTPAPTIASQPLSLRPEACRPAAGGAVHTRGLDFACDIYIWAPLAGTCGVLLLSLVITLYCLRHRRQGKHWTSTQRKADFQHPAGAVGPEPTDRGLQWRSSPAADAQEENLYAAVKHTQPEDGVEMDTRSPHDEDPQAVTYAEVKHSRPRREMASPPSPLSGEFLDTKDRQAEEDRQMDTEAAASEAPQDVTYAQLHSLTLRREATEPPPSQEGPSPAVPSIYATLAIHRRKRGSGEGRGSLLTCGDVEENPGPMALPVTALLLPLALLLHAARPEVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSGSTSGSGKPGSGEGSTKGDIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKIEVMYPPPYLDNEKSNGTIIHVKGKHLCPSPLFPGPSKPFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 31 - DNA, 2796 bp, synthetic construct
  source 1..2796: mol_type = other DNA
atggcactgccagtgaccgccctgctgctgcctctggccctgctgctgcacgcagccagaccccaagtgcaactagtccaatcaggtggaggcgtcgtgcaacctggagggtccctccgcgttagctgcgccgcatcaggcgttaccttgtcagactacggcatgcattgggttaggcaagcccccggcaaggggctcgaatggatggctttcattcggaatgacgggagcgataaatattacgcggattcagttaaagggcggttcaccatcagccgcgacaatagcaaaaagacggtctccttacagatgtccagcttgcgggccgaagacacggctgtatactattgtgctaaaaatggcgagagcggccccctggattactggtactttgacctgtggggcagaggcaccctggtcacggtgtcctctggaggaggaggctccggaggaggaggctctggcggcggcggcagcgacattgtaatgacccagtcaccctccttccttagtgcctcagtcggagaccgcgtgactatcacttgtcgtgcctcacacggaattaataactacctcgcttggtatcagcaaaaaccaggcaaggccccgaaactattgatctacgccgcatctactctgcagagcggagtaccgagcagatttagtggttccggcagcggaaccgagttcaccctaaccatttccagtttgcagccagaggatttcgcgacctattactgccagcaatacgattcatacccgccaactttcggaagaggtaccaaggttgaaatcaagactacgaccccagcacctagacctcccaccccagctccaactatagcttcccagccattgtctctccggccagaggcgtgtcgaccagccgctggaggggccgttcatacaagaggactcgatttcgcttgcgatatctacatatgggcccctcttgccgggacatgcggtgtcctgcttctaagcttggttattaccctctattgccataggtggtgctcaaacaaaaagaatgctgccgtcatggaccaggagagcgcgggcaatcggaccgcaaactcagaggactcagatgaacaagatccacaggaagtgacctacactcagctgaaccattgtgtgtttacacagcgcaagattactcgtccaagccagcgtcctaagacccccccgaccgatatcattgtgtataccgagcttcctaatgccgaatcccgcagcaaggtggtctcctgcccgcggagaaagcgtggatccggggaaggccgaggctcccttctaacatgtggagatgtcgaggaaaaccctggccctatggcgctgccagtcactgcattgttattgcctctggccctgcttctccatgcggcgcgcccagaagtgcagctggtcgagagcggaggcggactggttcaacccggaggcagcttgagactgtcctgcgcggccagcggcttcaacatcaaggatacctatatccactgggtgaggcaggctccaggaaagggcctggagtgggtggcaaggatttaccctactaatggatatacacgctacgctgattccgtgaagggacgctttacaatctcagcagatacatccaaaaacacggcctatttacagatgaatagtttgcgggccgaagacacggctgtatactattgttctcggtgggggggcgatggattttatgcgatggattactggggccagggcaccctggtaaccgtgtcaagcggctcaacatccgggtccggtaagccgggctccggcgaggggtctacaaagggagatatacagatgacacagtcccccagttccctgtccgcctcagtgggagaccgagtgacgattacctgtcgtgccagccaggacgtcaataccgccgtcgcttggtatcagcaaaaaccaggcaaggccccgaaactattgatctacagtgcctcttttctgtactccggggtgccgagcagatttagtggctccaggagcggaaccgatttcaccctaaccatttccagtttgcagccagaggatttcgcgacctattactgccagcaacactacaccacaccgccaactttcggacaaggaaccaaggttgaaatcaaaattgaagttatgtatcctcctccttacctagacaatgagaagagcaatggaaccattatccatgtgaaagggaaacacctttgtccaagtcccctatttcccgggccttcgaagcccttttgggtgctggtggtggttggtggagtcctggcttgctatagcttgctagtaacagtagcgtttattattttctgggtgaaacgcggccgcaagaaactgctctacatctttaaacagccgttcatgaggcctgtgcagacaacgcaggaagaggatggctgtagttgtcggtttccggaagaggaagaggggggctgcgagttgcgtgtcaaattttctcggtctgccgacgcccccgcgtaccagcaagggcagaaccagctttataatgagctgaatcttggacgacgggaggaatatgacgtgcttgacaagaggcgaggtagggaccctgagatggggggaaaacctcggaggaaaaacccacaggaaggcctgtataacgaactgcagaaggacaagatggctgaagcctactctgagattggaatgaaaggggaacgcagacgcggcaagggccatgatggcctctaccaaggtctaagcactgccaccaaggacacctatgacgcactccacatgcaagctctacctccccgttgataa

SEQ ID NO: 32 - protein, 930 aa, synthetic construct
MALPVTALLLPLALLLHAARPQVQLVQSGGGVVQPGGSLRVSCAASGVTLSDYGMHWVRQAPGKGLEWMAFIRNDGSDKYYADSVKGRFTISRDNSKKTVSLQMSSLRAEDTAVYYCAKNGESGPLDYWYFDLWGRGTLVTVSSGGGGSGGGGSGGGGSDIVMTQSPSFLSASVGDRVTITCRASHGINNYLAWYQQKPGKAPKLLIYAASTLQSGVPSRFSGSGSGTEFTLTISSLQPEDFATYYCQQYDSYPPTFGRGTKVEIKTTTPAPRPPTPAPTIASQPLSLRPEACRPAAGGAVHTRGLDFACDIYIWAPLAGTCGVLLLSLVITLYCHRWCSNKKNAAVMDQESAGNRTANSEDSDEQDPQEVTYTQLNHCVFTQRKITRPSQRPKTPPTDIIVYTELPNAESRSKVVSCPRRKRGSGEGRGSLLTCGDVEENPGPMALPVTALLLPLALLLHAARPEVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSGSTSGSGKPGSGEGSTKGDIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKIEVMYPPPYLDNEKSNGTIIHVKGKHLCPSPLFPGPSKPFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 33 - DNA, 3048 bp, synthetic construct
  source 1..3048: mol_type = other DNA
atggcactgccagtgaccgccctgctgctgcctctggccctgctgctgcacgcagccagaccccaggtgcagctgcagcagtctggacctgagctggtgaagccaggagcctccgtgaagatgtcttgcaaggccagcggctacaccttcacatcttatcacatccagtgggtgaagcagcggcccggacagggcctggagtggatcggatggatctacccaggcgacggctccacacagtataacgagaagttcaagggcaagaccacactgaccgccgataagagcagcagcaccgcctacatgctgctgagcagcctgaccagcgaggacagcgccatctacttttgcgccagggagggcacatactatgctatggactattggggccagggcaccagcgtgacagtgtctagcggaggaggaggctccggaggaggaggctctggcggcggcggcagcgacgtgctgatgacccagacaccactgagcctgcccgtgagcctgggcgatcaggtgagcatctcctgtagatcctctcagagcatcgtgcactccaacggcaatacctacctggagtggtatctgcagaagccaggccagtcccccaagctgctgatctataaggtgtctaatcggttcagcggcgtgcctgacagattttctggcagcggctccggcaccgacttcaccctgaagatcagccgggtggaggcagaggatctgggcgtgtactattgtttccagggctcccacgtgccacgcacctttggcggcggtaccaagctggagatcaagactacgaccccagcacctagacctcccaccccagctccaactatagcttcccagccattgtctctccggccagaggcgtgtcgaccagccgctggaggggccgttcatacaagaggactcgatttcgcttgcgatatctacatatgggcccctcttgccgggacatgcggtgtcctgcttctaagcttggttattaccctctattgcctgcgccacaggagacagggcaagcactggaccagcacccagcggaaggccgactttcagcaccctgccggcgccgtgggccctgagcctaccgacaggggcctgcagtggaggagctccccagccgccgatgcccaggaggagaatctgtacgccgccgtgaagcacacccagccagaggacggcgtggagatggacacccgctccccacacgacgaggatccacaggccgtgacctacgccgaggtgaagcacagccgccccagacgcgagatggccagcccacccagccccctgtccggcgagttcctggacaccaaggacaggcaggccgaggaggaccggcagatggacaccgaggccgccgcctccgaggccccccaggacgtgacctacgcccagctgcactccctgaccctgcggagagaggccaccgagcccccacccagccaggagggcccctcccccgccgtgcctagcatctacgccaccctggccatccaccggagaaagcgtggatccggggaaggccgaggctcccttctaacatgtggagatgtcgaggaaaaccctggccctatggcgctgccagtcactgcattgttattgcctctggccctgcttctccatgcggcgcgcccagaagtgcagctggtcgagagcggaggcggactggttcaacccggaggcagcttgagactgtcctgcgcggccagcggcttcaacatcaaggatacctatatccactgggtgaggcaggctccaggaaagggcctggagtgggtggcaaggatttaccctactaatggatatacacgctacgctgattccgtgaagggacgctttacaatctcagcagatacatccaaaaacacggcctatttacagatgaatagtttgcgggccgaagacacggctgtatactattgttctcggtgggggggcgatggattttatgcgatggattactggggccagggcaccctggtaaccgtgtcaagcggctcaacatccgggtccggtaagccgggctccggcgaggggtctacaaagggagatatacagatgacacagtcccccagttccctgtccgcctcagtgggagaccgagtgacgattacctgtcgtgccagccaggacgtcaataccgccgtcgcttggtatcagcaaaaaccaggcaaggccccgaaactattgatctacagtgcctcttttctgtactccggggtgccgagcagatttagtggctccaggagcggaaccgatttcaccctaaccatttccagtttgcagccagaggatttcgcgacctattactgccagcaacactacaccacaccgccaactttcggacaaggaaccaaggttgaaatcaaaattgaagttatgtatcctcctccttacctagacaatgagaagagcaatggaaccattatccatgtgaaagggaaacacctttgtccaagtcccctatttcccgggccttcgaagcccttttgggtgctggtggtggttggtggagtcctggcttgctatagcttgctagtaacagtagcgtttattattttctgggtgaaacgcggccgcaagaaactgctctacatctttaaacagccgttcatgaggcctgtgcagacaacgcaggaagaggatggctgtagttgtcggtttccggaagaggaagaggggggctgcgagttgcgtgtcaaattttctcggtctgccgacgcccccgcgtaccagcaagggcagaaccagctttataatgagctgaatcttggacgacgggaggaatatgacgtgcttgacaagaggcgaggtagggaccctgagatggggggaaaacctcggaggaaaaacccacaggaaggcctgtataacgaactgcagaaggacaagatggctgaagcctactctgagattggaatgaaaggggaacgcagacgcggcaagggccatgatggcctctaccaaggtctaagcactgccaccaaggacacctatgacgcactccacatgcaagctctacctccccgttgataa

SEQ ID NO: 34 - protein, 1014 aa, synthetic construct
MALPVTALLLPLALLLHAARPQVQLQQSGPELVKPGASVKMSCKASGYTFTSYHIQWVKQRPGQGLEWIGWIYPGDGSTQYNEKFKGKTTLTADKSSSTAYMLLSSLTSEDSAIYFCAREGTYYAMDYWGQGTSVTVSSGGGGSGGGGSGGGGSDVLMTQTPLSLPVSLGDQVSISCRSSQSIVHSNGNTYLEWYLQKPGQSPKLLIYKVSNRFSGVPDRFSGSGSGTDFTLKISRVEAEDLGVYYCFQGSHVPRTFGGGTKLEIKTTTPAPRPPTPAPTIASQPLSLRPEACRPAAGGAVHTRGLDFACDIYIWAPLAGTCGVLLLSLVITLYCLRHRRQGKHWTSTQRKADFQHPAGAVGPEPTDRGLQWRSSPAADAQEENLYAAVKHTQPEDGVEMDTRSPHDEDPQAVTYAEVKHSRPRREMASPPSPLSGEFLDTKDRQAEEDRQMDTEAAASEAPQDVTYAQLHSLTLRREATEPPPSQEGPSPAVPSIYATLAIHRRKRGSGEGRGSLLTCGDVEENPGPMALPVTALLLPLALLLHAARPEVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSGSTSGSGKPGSGEGSTKGDIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKIEVMYPPPYLDNEKSNGTIIHVKGKHLCPSPLFPGPSKPFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 35 - DNA, 2796 bp, synthetic construct
  source 1..2796: mol_type = other DNA
atggcactgccagtgaccgccctgctgctgcctctggccctgctgctgcacgcagccagaccccaggtgcagctgcagcagtctggacctgagctggtgaagccaggagcctccgtgaagatgtcttgcaaggccagcggctacaccttcacatcttatcacatccagtgggtgaagcagcggcccggacagggcctggagtggatcggatggatctacccaggcgacggctccacacagtataacgagaagttcaagggcaagaccacactgaccgccgataagagcagcagcaccgcctacatgctgctgagcagcctgaccagcgaggacagcgccatctacttttgcgccagggagggcacatactatgctatggactattggggccagggcaccagcgtgacagtgtctagcggaggaggaggctccggaggaggaggctctggcggcggcggcagcgacgtgctgatgacccagacaccactgagcctgcccgtgagcctgggcgatcaggtgagcatctcctgtagatcctctcagagcatcgtgcactccaacggcaatacctacctggagtggtatctgcagaagccaggccagtcccccaagctgctgatctataaggtgtctaatcggttcagcggcgtgcctgacagattttctggcagcggctccggcaccgacttcaccctgaagatcagccgggtggaggcagaggatctgggcgtgtactattgtttccagggctcccacgtgccacgcacctttggcggcggtaccaagctggagatcaagactacgaccccagcacctagacctcccaccccagctccaactatagcttcccagccattgtctctccggccagaggcgtgtcgaccagccgctggaggggccgttcatacaagaggactcgatttcgcttgcgatatctacatatgggcccctcttgccgggacatgcggtgtcctgcttctaagcttggttattaccctctattgccataggtggtgctcaaacaaaaagaatgctgccgtcatggaccaggagagcgcgggcaatcggaccgcaaactcagaggactcagatgaacaagatccacaggaagtgacctacactcagctgaaccattgtgtgtttacacagcgcaagattactcgtccaagccagcgtcctaagacccccccgaccgatatcattgtgtataccgagcttcctaatgccgaatcccgcagcaaggtggtctcctgcccgcggagaaagcgtggatccggggaaggccgaggctcccttctaacatgtggagatgtcgaggaaaaccctggccctatggcgctgccagtcactgcattgttattgcctctggccctgcttctccatgcggcgcgcccagaagtgcagctggtcgagagcggaggcggactggttcaacccggaggcagcttgagactgtcctgcgcggccagcggcttcaacatcaaggatacctatatccactgggtgaggcaggctccaggaaagggcctggagtgggtggcaaggatttaccctactaatggatatacacgctacgctgattccgtgaagggacgctttacaatctcagcagatacatccaaaaacacggcctatttacagatgaatagtttgcgggccgaagacacggctgtatactattgttctcggtgggggggcgatggattttatgcgatggattactggggccagggcaccctggtaaccgtgtcaagcggctcaacatccgggtccggtaagccgggctccggcgaggggtctacaaagggagatatacagatgacacagtcccccagttccctgtccgcctcagtgggagaccgagtgacgattacctgtcgtgccagccaggacgtcaataccgccgtcgcttggtatcagcaaaaaccaggcaaggccccgaaactattgatctacagtgcctcttttctgtactccggggtgccgagcagatttagtggctccaggagcggaaccgatttcaccctaaccatttccagtttgcagccagaggatttcgcgacctattactgccagcaacactacaccacaccgccaactttcggacaaggaaccaaggttgaaatcaaaattgaagttatgtatcctcctccttacctagacaatgagaagagcaatggaaccattatccatgtgaaagggaaacacctttgtccaagtcccctatttcccgggccttcgaagcccttttgggtgctggtggtggttggtggagtcctggcttgctatagcttgctagtaacagtagcgtttattattttctgggtgaaacgcggccgcaagaaactgctctacatctttaaacagccgttcatgaggcctgtgcagacaacgcaggaagaggatggctgtagttgtcggtttccggaagaggaagaggggggctgcgagttgcgtgtcaaattttctcggtctgccgacgcccccgcgtaccagcaagggcagaaccagctttataatgagctgaatcttggacgacgggaggaatatgacgtgcttgacaagaggcgaggtagggaccctgagatggggggaaaacctcggaggaaaaacccacaggaaggcctgtataacgaactgcagaaggacaagatggctgaagcctactctgagattggaatgaaaggggaacgcagacgcggcaagggccatgatggcctctaccaaggtctaagcactgccaccaaggacacctatgacgcactccacatgcaagctctacctccccgttgataa

SEQ ID NO: 36 - protein, 931 aa, synthetic construct
MALPVTALLLPLALLLHAARPQVQLQQSGPELVKPGASVKMSCKASGYTFTSYHIQWVKQRPGQGLEWIGWIYPGDGSTQYNEKFKGKTTLTADKSSSTAYMLLSSLTSEDSAIYFCAREGTYYAMDYWGQGTSVTVSSGGGGSGGGGSGGGGSDVLMTQTPLSLPVSLGDQVSISCRSSQSIVHSNGNTYLEWYLQKPGQSPKLLIYKVSNRFSGVPDRFSGSGSGTDFTLKISRVEAEDLGVYYCFQGSHVPRTFGGGTKLEIKTTTPAPRPPTPAPTIASQPLSLRPEACRPAAGGAVHTRGLDFACDIYIWAPLAGTCGVLLLSLVITLYCHRWCSNKKNAAVMDQESAGNRTANSEDSDEQDPQEVTYTQLNHCVFTQRKITRPSQRPKTPPTDIIVYTELPNAESRSKVVSCPRRKRGSGEGRGSLLTCGDVEENPGPMALPVTALLLPLALLLHAARPEVQLVESGGGLLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSGSTSGSGKPGSGEGSTKGDIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKIEVMYPPPYLDNEKSNGTIIHVKGKHLCPSPLFPGPSKPFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 37 - protein, 112 aa, synthetic construct
DVLMTQTPLSLPVSLGDQVSISCRSSQSIVHSNGNTYLEWYLQKPGQSPKLLIYKVSNRFSGVPDRFSGSGSGTDFTLKISRVEAEDLGVYYCFQGSHVPRTFGGGTKLEIK

SEQ ID NO: 38 - protein, 118 aa, synthetic construct
QVQLQQSGPELVKPGASVKMSCKASGYTFTSYHIQWVKQRPGQGLEWIGWIYPGDGSTQYNEKFKGKTTLTADKSSSTAYMLLSSLTSEDSAIYFCAREGTYYAMDYWGQGTSVTVSS

SEQ ID NO: 39 - protein, 107 aa, synthetic construct
DIVMTQSPSFLSASVGDRVTITCRASHGINNYLAWYQQKPGKAPKLLIYAASTLQSGVPSRFSGSGSGTEFTLTISSLQPEDFATYYCQQYDSYPPTFGRGTKVEIK

SEQ ID NO: 40 - protein, 123 aa, synthetic construct
QVQLVQSGGGVVQPGGSLRVSCAASGVTLSDYGMHWVRQAPGKGLEWMAFIRNDGSDKYYADSVKGRFTISRDNSKKTVSLQMSSLRAEDTAVYYCAKNGESGPLDYWYFDLWGRGTLVTVSS

SEQ ID NO: 41 - protein, 107 aa, synthetic construct
DVVMTQSPSSLSASVGDRVTITCQASQDISNYLNWYQQKPGKAPKLLIYDASNLETGVPSRFSGSGSGTDFTFTISSLQPEDFATYYCQQYSSFPLTFGGGTKVDIK

SEQ ID NO: 42 - protein, 123 aa, synthetic construct; identical to SEQ ID NO: 40

SEQ ID NO: 43 - protein, 107 aa, synthetic construct
DVVMTQSPSSLSASVGDRVTITCQASQDISNYLNWYQQKPGKAPKLLIYDASNLETGVPSRFSGSGSGTDFTFTISSLQPEDIATYYCQQYDNLPPTFGGGTKLEIV

SEQ ID NO: 44 - protein, 123 aa, synthetic construct; identical to SEQ ID NO: 40

SEQ ID NO: 45 - protein, 108 aa, synthetic construct
SIVMTQTPKFLLVSAGDRVTITCKASQSVSNDVAWYQQKPGQSPKLLIYYASNRYTGVPDRFTGSGYGTDFTFTISTVQAEDLAVYFCQQDYSSPPWTFGGGTKLEIR

SEQ ID NO: 46 - protein, 120 aa, synthetic construct
QVQLKQSGPGLVQPSQSLSLTCTVSGFSLTSYGVHWVRQPPGKGLEWLGVIWSGGSTDYNAAFISRLSIRKDNSKSQVFFKMNSLQADDTAIYYCARTFTTSTSAWFAYWGQGTLVTVSA

SEQ ID NO: 47 - protein, 107 aa, synthetic construct
DIQMTQSPASQSASLGESVTITCLASQTIGTWLAWYQQKPGKSPQLLIYAATSLADGVPSRFSGSGSGTKFSLKIRTLQAEDFVSYYCQQLYSKPYTFGGGTKLEIK

SEQ ID NO: 48 - protein, 119 aa, synthetic construct
EVQLQQSGAELVKPGASVKLSCTPSGFNVKDTYIHWVKQRPKQGLEWIGRIDPSDGDIKYDPKFQGKATITADTSSNTVSLQLSSLTSEDTAVYYCARWFGDYGAMNYWGQGTSVTVSS

SEQ ID NO: 49 - protein, 113 aa, synthetic construct
DIVMTQSPDSLAVSLGERATISCKSSQSVLYSSNNKNYLAWYQQKLGQPPKLLIYWASTRESGVPDRFSGSGSGTNFTLTISSLQAENVAVYYCQQYYGTPFTFGGGTKVEIK

SEQ ID NO: 50 - protein, 125 aa, synthetic construct
QVQLVQSGAEVKKPGASVKVSCKASGYTFTDYYLHWVRQAPGQGLEWMGWINPYTGGTNYAQKFQGRVTMTRDASISTVYMELSGLTSDDTAVHFCARAGASYYDFWSGWVFDYWGQGTLVTVSS

SEQ ID NO: 51 - protein, 107 aa, synthetic construct
DIQMTQSPSSLSASVGDRVTITCRASQSISTYLNWYQQKPGKAPKLLIYAASSLQSGVPSRFSGSGSGTDFTLTISSLQPEDFATYQCQQSYSTPFTFGGGTKVEIK

SEQ ID NO: 52 - protein, 124 aa, synthetic construct
QVQLQESGPGLVKPSETLSLTCTVSGGSISSYYWSWIRQPAGKGLEWIGRIYISGGTNYNPSLKSRVTMSVDTSKNQVSLKLSSVTAADTAVYYCARDILGGVSGWSHYGMDVWGQGTTVTVSS

SEQ ID NO: 53 - protein, 110 aa, synthetic construct
QSALTQPPSASGSPGQSVTISCTGTSSDVGGYKYVSWYQHHPDKAPKLMIYEVNKRPSGVPDRFSGSKSDNTASLTVSGLQAEDEADYYCSSYAGSNNWVFGGGTKLTVL

SEQ ID NO: 54 - protein, 118 aa, synthetic construct
QVQLVESGGGVVQPGGSLRLSCAASGFTFSTYGMHWVRQAPGKGLEWAASVSYDGSNKYYADSGQGRFTISRDTSMNSLYLQVNSLRDETAVYYCAIGIYGAYSFDYWGQGTLVTVSS

SEQ ID NO: 55 - protein, 110 aa, synthetic construct; identical to SEQ ID NO: 53

SEQ ID NO: 56 - protein, 119 aa, synthetic construct
QVQLVESGGGVVQPGGSLRLSCAASGFTFSTYGMHWVRQAPGKGLEWVASISYDGSNKYYADSGQGRFTISRDTSKNSLYLQMNSLRAEDTAVYYCAIGIYGAYSFDYWGQGTLVTVSS

SEQ ID NO: 57 - protein, 112 aa, synthetic construct
DVVMTQTPLSLSVTPGQPASISCRSSQSIVHSNGNTYLEWYLQKPGQSPQLLIYKVSNRFSGVPDRFSGSGSGTDFTLKISRVEAEDVGVYYCFQGSHVPRTFGGGTKVEIK

SEQ ID NO: 58 - protein, 118 aa, synthetic construct
QVQLVQSGAEVKKPGSSVKVSCKASGGTFSSYHIQWVRQAPGQGLEWMGWIYPGDGSTQYNEKFKGRVTITADKSTSTAYMELSSLRSEDTAVYYCAREGTYYAMDYWGQGTLVTVSS

SEQ ID NO: 59 - protein, 112 aa, synthetic construct; identical to SEQ ID NO: 57

SEQ ID NO: 60 - protein, 118 aa, synthetic construct
QVQLVQSGAEVKKPGSSVKVSCKASGYTFTSYHIQWVRQAPGQGLEWMGWIYPGDGSTQYNEKFKGRVTITADKSTSTAYMELSSLRSEDTAVYYCAREGTYYAMDYWGQGTLVTVSS

SEQ ID NO: 61 - protein, 112 aa, synthetic construct; identical to SEQ ID NO: 57

SEQ ID NO: 62 - protein, 118 aa, synthetic construct
QVQLVQSGAEVKKPGSSVKVSCKASGYTFTSYHIQWVRQAPGQGLEWIGWIYPGDGSTQYNEKFKGRVTITADKSTSTAYMELSSLRSEDTAVYYCAREGTYYAMDYWGQGTLVTVSS

SEQ ID NO: 63 - protein, 112 aa, synthetic construct; identical to SEQ ID NO: 57

SEQ ID NO: 64 - protein, 118 aa, synthetic construct
QVQLVQSGAEVKKPGSSVKVSCKASGYTFTSYHIQWVRQAPGQGLEWMGWIYPGDGSTQYNEKFKGRTTITADKSTSTAYMELSSLRSEDTAVYYCAREGTYYAMDYWGQGTLVTVSS

SEQ ID NO: 65 - protein, 112 aa, synthetic construct; identical to SEQ ID NO: 57

SEQ ID NO: 66 - protein, 118 aa, synthetic construct
QVQLVQSGAEVKKPGSSVKVSCKASGYTFTSYHIQWVRQAPGQGLEWMGWIYPGDGSTQYNEKFKGRVTLTADKSTSTAYMELSSLRSEDTAVYYCAREGTYYAMDYWGQGTLVTVSS

SEQ ID NO: 67 - protein, 112 aa, synthetic construct; identical to SEQ ID NO: 57

SEQ ID NO: 68 - protein, 118 aa, synthetic construct
QVQLVQSGAEVKKPGSSVKVSCKASGYTFTSYHIQWVRQAPGQGLEWMGWIYPGDGSTQYNEKFKGRTTLTADKSTSTAYMELSSLRSEDTAVYYCAREGTYYAMDYWGQGTLVTVSS

SEQ ID NO: 69 - protein, 112 aa, synthetic construct; identical to SEQ ID NO: 57

SEQ ID NO: 70 - protein, 118 aa, synthetic construct
QVQLVQSGAEVKKPGASVKVSCKASGYTFTSYHIQWVRQAPGQRLEWMGWIYPGDGSTQYNEKFKGRVTITRDTSASTAYMELSSLRSEDTAVYYCAREGTYYAMDYWGQGTLVTVSS

SEQ ID NO: 71 - protein, 112 aa, synthetic construct; identical to SEQ ID NO: 57

SEQ ID NO: 72 - protein, 118 aa, synthetic construct
QVQLVQSGAEVKKPGASVKVSCKASGYTFTSYHIQWVRQAPGQRLEWIGWIYPGDGSTQYNEKFKGRVTITRDTSASTAYMELSSLRSEDTAVYYCAREGTYYAMDYWGQGTLVTVSS

SEQ ID NO: 73 - protein, 112 aa, synthetic construct; identical to SEQ ID NO: 57

SEQ ID NO: 74 - protein, 118 aa, synthetic construct
QVQLVQSGAEVKKPGASVKVSCKASGYTFTSYHIQWVRQAPGQRLEWMGWIYPGDGSTQYNEKFKGRTTITRDTSASTAYMELSSLRSEDTAVYYCAREGTYYAMDYWGQGTLVTVSS

SEQ ID NO: 75 - protein, 112 aa, synthetic construct; identical to SEQ ID NO: 57

SEQ ID NO: 76 - protein, 118 aa, synthetic construct
QVQLVQSGAEVKKPGASVKVSCKASGYTFTSYHIQWVRQAPGQRLEWMGWIYPGDGSTQYNEKFKGRVTLTRDTSASTAYMELSSLRSEDTAVYYCAREGTYYAMDYWGQGTLVTVSS

SEQ ID NO: 77 - protein, 112 aa, synthetic construct; identical to SEQ ID NO: 57

SEQ ID NO: 78 - protein, 118 aa, synthetic construct
QVQLVQSGAEVKKPGASVKVSCKASGYTFTSYHIQWVRQAPGQRLEWMGWIYPGDGSTQYNEKFKGRVTITADTSASTAYMELSSLRSEDTAVYYCAREGTYYAMDYWGQGTLVTVSS

SEQ ID NO: 79 - protein, 112 aa, synthetic construct; identical to SEQ ID NO: 57

SEQ ID NO: 80 - protein, 118 aa, synthetic construct
QVQLVQSGAEVKKPGASVKVSCKASGYTFTSYHIQWVRQAPGQRLEWMGWIYPGDGSTQYNEKFKGRVTITRDKSASTAYMELSSLRSEDTAVYYCAREGTYYAMDYWGQGTLVTVSS

SEQ ID NO: 81 - protein, 15 aa, synthetic construct
GGGGSGGGGSGGGGS

SEQ ID NO: 82 - protein, 18 aa, synthetic construct
GSTSGSGKPGSGEGSTKG

SEQ ID NO: 83 - protein, 19 aa, synthetic construct
DFQWREKTPEPPVPCVPEQ

SEQ ID NO: 84 - protein, 45 aa, synthetic construct
TTTPAPRPPTPAPTIASQPLSLRPEACRPAAGGAVHTRGLDFACD

SEQ ID NO: 85 - protein, 39 aa, synthetic construct
IEVMYPPPYLDNEKSNGTIIHVKGKHLCPSPLFPGPSKP

SEQ ID NO: 86 - protein, 26 aa, synthetic construct
TERRAEVPTAHPSPSPRPAGQFQTLV

SEQ ID NO: 87 - protein, 240 aa, synthetic construct
VSKKPSLSVQPGPIVAPEETLTLQCGSDAGYNRFVLYKDGERDFLQLAGAQPQAGLSQANFTLGPVSRSYGGQYRCYGAHNLSSEWSAPSDPLDILIAGQFYDRVSLSVQPGPTVASGENVTLLCQSQGWMQTFLLTKEGAADDPWRLRSTYQSQKYQAEFPMGPVTSAHAGTYRCYGSQSSKPYLLTHPSDPLELVVSGPSGGPSSPTTGPTSTSGPEDQPLTPTGSDPQSGLGRHLGV

SEQ ID NO: 88 - protein, 149 aa, synthetic construct
PLDILIAGQFYDRVSLSVQPGPTVASGENVTLLCQSQGWMQTFLLTKEGAADDPWRLRSTYQSQKYQAEFPMGPVTSAHAGTYRCYGSQSSKPYLLTHPSDPLELVVSGPSGGPSSPTTGPTSTSGPEDQPLTPTGSDPQSGLGRHLGV

SEQ ID NO: 89 - protein, 52 aa, synthetic construct
HPSDPLELVVSGPSGGPSSPTTGPTSTSGPEDQPLTPTGSDPQSGLGRHLGV

SEQ ID NO: 90 - protein, 36 aa, synthetic construct
PSSPTTGPTSTSGPEDQPLTPTGSDPQSGLGRHLGV

SEQ ID NO: 91 - protein, 30 aa, synthetic construct
GPTSTSGPEDQPLTPTGSDPQSGLGRHLGV

SEQ ID NO: 92 - protein, 8 aa, synthetic construct
GLGRHLGV

SEQ ID NO: 93 - protein, 31 aa, synthetic construct
LNVTYVPQNPTTGIFPGDGSGKQETRAGVVH

SEQ ID NO: 94 - protein, 40 aa, synthetic construct
PYEWSKSSDPLLVSVTGNPSNSWPSPTEPSSKTGNPRHLH

SEQ ID NO: 95 - protein, 24 aa, synthetic construct
IYIWAPLAGTCGVLLLSLVITLYC

SEQ ID NO: 96 - protein, 27 aa, synthetic construct
FWVLVVVGGVLACYSLLVTVAFIIFWV

SEQ ID NO: 97 - protein, 21 aa, synthetic construct
VGVVGGLLGSLVLLVWVLAVI

SEQ ID NO: 98 - protein, 21 aa, synthetic construct
VIGILVAVILLLLLLLLLFLI

SEQ ID NO: 99 - protein, 23 aa, synthetic construct
GAIGGAGVTALLALCLCLIFFIV

SEQ ID NO: 100 - protein, 19 aa, synthetic construct
ILIGTSVVIILFILLFFLL

SEQ ID NO: 101 - protein, 97 aa, synthetic construct
CSRAARGTIGARRTGQPLKEDPSAVPVFSVDYGELDFQWREKTPEPPVPCVPEQTEYATIVFPSGMGTSSPARRGSADGPRSAQPLRPEDGHCSWPL

SEQ ID NO: 102 - protein, 84 aa, synthetic construct
HRWCSNKKNAAVMDQESAGNRTANSEDSDEQDPQEVTYTQLNHCVFTQRKITRPSQRPKTPPTDIIVYTELPNAESRSKVVSCP

SEQ ID NO: 103 - protein, 84 aa, synthetic construct
HRWCSNKKNAAVMDQESAGNRTANSEDSDEQDPQEVTYTQLNHCVFTQRKITRPSQRPKTPPTDIIVYAELPNAESRSKVVSCP

SEQ ID NO: 104 - protein, 76 aa, synthetic construct
HRWCCNKKNAVVMDQEPAGNRTVNREDSDEQDPQEVTYAQLNHCVFTQRKITRPSQRPKTPPTDIIVYTELPNAEP

SEQ ID NO: 105 - protein, 114 aa, synthetic construct
RWCSKKKDAAVMNQEPAGHRTVNREDSDEQDPQEVTYAQLDHCIFTQRKITGPSQRSKRPSTDTSVCIELPNAEPRALSPAHEHHSQALMGSSRETTALSQTQLASSNVPAAGI

SEQ ID NO: 106 - protein, 116 aa, synthetic construct
LHCCCSNKKNAAVMDQEPAGDRTVNREDSDDQDPQEVTYAQLDHCVFTQTKITSPSQRPKTPPTDTTMYMELPNAKPRSLSPAHKHHSQALRGSSRETTALSQNRVASSHVPAAGI

SEQ ID NO: 107 - protein, 84 aa, synthetic construct
HLWCSNKKNAAVMDQEPAGNRTANSEDSDEQDPEEVTYAQLDHCVFTQRKITRPSQRPKTPPTDTILYTELPNAKPRSKVVSCP

SEQ ID NO: 108 - protein, 95 aa, synthetic construct
YRWCSNKKNAAVMDQEPAGDRTVNRQDSDEQDPQEVTYAQLDHCVFIQRKISRPSQRPKTPLTDTSVYTELPNAEPRSKVVSCPRAPQSGLEGVF

SEQ ID NO: 109 - protein, 67 aa, synthetic construct
HRWCANKKNAVVMDQEPAGNRTVNREDSDEQDPQEVTYAQLNHCVFTQRKITRPSQRPKTPPTDTSV

SEQ ID NO: 110 - protein, 101 aa, synthetic construct
HRQNQIKQGPPRSKDEEQKPQQRPDLAVDVLERTADKATVNGLPEKDRETDTSALAAGSSQEVTYAQLDHWALTQRTARAVSPQSTKPMAESITYAAVARH

SEQ ID NO: 111 - protein, 141 aa, synthetic construct
KLQRRWKRTQSQQGLQENSSGQSFFVRNKKVRRAPLSEGPHSLGCYNPMMEDGISYTTLRFPEMNIPRTGDAESSEMQRPPPDCDDTVTYSALHKRQVGDYENVIPDFPEDEGIHYSELIQFGVGERPQAQENVDYVILKH

SEQ ID NO: 112 - protein, 82 aa, synthetic construct
KTHRRKAARTAVGRNDTHPTTGSASPKHQKKSKLHGPTETSSCSGAAPTVEMDEELHYASLNFHGMNPSKDTSTEYSEVRTQ

SEQ ID NO: 113 - protein, 89 aa, synthetic construct
KARRKQAAGRPEKMDDEDPIMGTITSGSRKKPWPDSPGDQASPPGDAPPLEEQKELHYASLSFSEMKSREPKDQEAPSTTEYSEIKTSK

SEQ ID NO: 114 - protein, 85 aa, synthetic construct
RVKTRRKKAAQPVQNTDDVNPVMVSGSRGHQHQFQTGIVSDHPAEAGPISEDEQELHYAVLHFHKVQPQEPKVTDTEYSEIKIHK

SEQ ID NO: 115 - protein, 91 aa, synthetic construct
RSCRKKSARPAADVGDIGMKDANTIRGSASQGNLTESWADDNPRHHGLAAHSSGEEREIQYAPLSFHKGEPQDLSGQEATNNEYSEIKIPK

SEQ ID NO: 116 - protein, 115 aa, synthetic construct
RSCRKKSARPAAGVGDTGMEDAKAIRGSASQGPLTESWKDGNPLKKPPPAVAPSSGEEGELHYATLSFHKVKPQDPQGQEATDSEYSEIKIHKRETAETQACLRNHNPSSKEVRG

SEQ ID NO: 117 - protein, 94 aa, synthetic construct
VRSCRKKSARPAAGVGDTGIEDANAVRGSASQGPLTEPWAEDSPPDQPPPASARSSVGEGELQYASLSFQMVKPWDSRGQEATDTEYSEIKIHR

SEQ ID NO: 118 - protein, 126 aa, synthetic construct
KILPKRRTQTETPRPRFSRHSTILDYINVVPTAGPLAQKRNQKATPNSPRTPLPPGAPSPESKKNQKKQYQLPSFPEPKSSTQAPESQESQEELHYATLNFPGVRPRPEARMPKGTQADYAEVKFQ

SEQ ID NO: 119 - protein, 114 aa, synthetic construct
KICRKEARKRAAAEQDVPSTLGPISQGHQHECSAGSSQDHPPPGAATYTPGKGEEQELHYASLSFQGLRLWEPADQEAPSTTEYSEIKIHTGQPLRGPGFGLQLEREMSGMVPK

SEQ ID NO: 120 - protein, 93 aa, synthetic construct
RSCRKKSARPAVGVGDTGMEDANAVRGSASQGPLIESPADDSPPHHAPPALATPSPEEGEIQYASLSFHKARPQYPQEQEAIGYEYSEINIPK

SEQ ID NO: 121 - protein, 118 aa, synthetic construct
KCYFLRKAKAKQMPVEMSRPAVPLLNSNNEKMSDPNMEANSHYGHNDDVRNHAMKPINDNKEPLNSDVQYTEVQVSSAESHKDLGKKDTETVYSEVRKAVPDAVESRYSRTEGSLDGT

SEQ ID NO: 122 - protein, 61 aa, synthetic construct
KVNGCRKYKLNKTESTPVVEEDEMQPYASYTEKNNPLYDTTNKVKASEALQSEVDTDLHTL

SEQ ID NO: 123 - protein, 101 aa, synthetic construct
GLKRKIGRRSARDPLRSLPSPLPQEFTYLNSPTPGQLQPIYENVNVVSGDEVYSLAYYNQPEQESVAAETLGTHMEDKVSLDIYSRLRKANITDVDYEDAM

SEQ ID NO: 124 - protein, 86 aa, synthetic construct
HKISGESSATNEPRGASRPNPQEFTYSSPTPDMEELQPVYVNVGSVDVDVVYSQVWSMQQPESSANIRTLLENKDSQVIYSSVKKS

SEQ ID NO: 125 - protein, 140 aa, synthetic construct
HYARARRKPGGLSATGTSSHSPSECQEPSSSRPSRIDPQEPTHSKPLAPMELEPMYSNVNPGDSNPIYSQIWSIQHTKENSANCPMMHQEHEELTVLYSELKKTHPDDSAGEASSRGRAHEEDDEENYENVPRVLLASDH

SEQ ID NO: 126 - protein, 107 aa, synthetic construct
HCWRRRKSGVGFLGDETRLPPAPGPGESSHSICPAQVELQSLYVDVHPKKGDLVYSEIQTTQLGEEEEANTSRTLLEDKDVSVVYSEVKTQHPDNSAGKISSKDEES

SEQ ID NO: 127 - protein, 105 aa, synthetic construct
LSRKAGRKPASDPARSPSDSDSQEPTYHNVPAWEELQPVYTNANPRGENVVYSEVRIIQEKKKHAVASDPRHLRNKGSPIIYSEVKVASTPVSGSLFLASSAPHR

SEQ ID NO: 128 - protein, 77 aa, synthetic construct
QLRRRGKTNHYQTTVEKKSLTIYAQVQKPGPLQKKLDSFPAQDPCTTIYVAATEPVPESVQETNSITVYASVTLPES

SEQ ID NO: 129 - protein, 99 aa, synthetic construct
RLFKRRQGRIFPEGSCLNTFTKNPYAASKKTIYTYIMASRNTQPAESRIYDEILQSKVLPSKEEPVNTVYSEVQFADKMGKASTQDSKPPGTSSYEIVI

SEQ ID NO: 130 - protein, 111 aa, synthetic construct
RRHQGKQNELSDTAGREINLVDAHLKSEQTEASTRQNSQVLLSETGIYDNDPDLCFRMQEGSEVYSNPCLEENKPGIVYASLNHSVIGPNSRLARNVKEAPTEYASICVRS

SEQ ID NO: 131 - protein, 54 aa, synthetic construct
HLWRRQWRPRRFSALEQGIHPPQAQSKIEELEQEPEPEPEPEPEPEPEPEPEQL

SEQ ID NO: 132 - protein, 120 aa, synthetic construct
WRRKRKEKQSETSPKEFLTIYEDVKDLKTRRNHEQEQTFPGGGSTIYSMIQSQSSAPTSQEPAYTLYSLIQPSRKSGSRKRNHSPSFNSTIYEVIGKSQPKAQNPARLSRKELENFDVYS

SEQ ID NO: 133 - protein, 135 aa, synthetic construct
GCINITSSASQEGTRLNLICTVWHKKEEAEGFVVFLCKDRSGDCSPETSLKQLRLKRDPGIDGVGEISSQLMFTISQVTPLHSGTYQCCARSQKSGIRLQGHFFSILFTETGNYTVTGLKQRQHLEFSHNEGTLS

SEQ ID NO: 134 - protein, 74 aa, synthetic construct
HFGKTGRASDQRDLTEHKPSVSNHTQDHSNDPPNKMNEVTYSTLNFEAQQPTQPTSASPSLTATEIIYSEVKKQ

SEQ ID NO: 135 - protein, 78 aa, synthetic construct
FKWYSHSKEKIQNLSLISLANLPPSGLANAVAEGIRSEENIYTIEENVYEVEEPNEYYCYVSSRQQPSQPLGCRFAMP

SEQ ID NO: 136 - protein, 96 aa, synthetic construct
YKQRQAASNRRAQELVRMDSNIQGIENPGFEASPPAQGIPEAKVRHPLSYVAQRQPSESGRHLLSEPSTPLSPPGPGDVFFPSLDPVPDSPNFEVI

SEQ ID NO: 137 - protein, 82 aa, synthetic construct
LTRKKKALRIHSVEGDLRRKSAGQEEWSPSAPSPPGSCVQAEAAPAGLCGEQRGEDCAELHDYFNVLSYRSLGNCSFFTETG

SEQ ID NO: 138 - protein, 110 aa, synthetic construct
RIRQKKAQGSTSSTRLHEPEKNAREITQDTNDITYADLNLPKGKKPAPQAAEPNNHTEYASIQTSPQPASEDTLTYADLDMVHLNRTPKQPAPKPEPSFSEYASVQVPRK

SEQ ID NO: 139 - protein, 70 aa, synthetic construct
VVALIYCRKKRISALPGYPECREMGETLPEKPANPTNPDEADKVGAENTITYSLLMHPDALEEPDDQNRI

SEQ ID NO: 140 - protein, 93 aa, synthetic construct
KKLVKKFRQKKQRQWIGPTGMNQNMSFHRNHTATVRSHAENPTASHVDNEYSQPPRNSHLSAYPALEGALHRSSMQPDNSSDSDYDLHGAQRL

SEQ ID NO: 141 - protein, 98 aa, synthetic construct
RMFQKWIKAGDHSELSQNPKQAATQSELHYANLELLMWPLQEKPAPPREVEVEYSTVASPREELHYASVVFDSNTNRIAAQRPREEEPDSDYSVIRKT

SEQ ID NO: 142 - protein, 113 aa, synthetic construct
WRMMKYQQKAAGMSPEQVLQPLEGDLCYADLTLQLAGTSPQKATTKLSSAQVDQVEVEYVTMASLPKEDISYASLTLGAEDQEPTYCNMGHLSSHLPGRGPEEPTEYSTISRP

SEQ ID NO: 143 - protein, 168 aa, synthetic construct
LRHRRQGKHWTSTQRKADFQHPAGAVGPEPTDRGLQWRSSPAADAQEENLYAAVKHTQPEDGVEMDTRSPHDEDPQAVTYAEVKHSRPRREMASPPSPLSGEFLDTKDRQAEEDRQMDTEAAASEAPQDVTYAQLHSLTLRREATEPPPSQEGPSPAVPSIYATLAIH

SEQ ID NO: 144 - protein, 116 aa, synthetic construct
LRHRRQGKHWTSTQRKADFQHPAGAVGPEPTDRGLQWRSSPAADAQEENLYAAVKDTQPEDGVEMDTRAAASEAPQDVTYAQLHSLTLRRKATEPPPSQEREPPAEPSIYATLAIH

SEQ ID NO: 145 - protein, 167 aa, synthetic construct
RRQRHSKHRTSDQRKTDFQRPAGAAETEPKDRGLLRRSSPAADVQEENLYAAVKDTQSEDRVELDSQSPHDEDPQAVTYAPVKHSSPRREMASPPSSLSGEFLDTKDRQVEEDRQMDTEAAASEASQDVTYAQLHSLTLRRKATEPPPSQEGEPPAEPSIYATLAIH

SEQ ID NO: 146 - protein, 168 aa, synthetic construct
QHWRQGKHRTLAQRQADFQRPPGAAEPEPKDGGLQRRSSPAADVQGENFCAAVKNTQPEDGVEMDTRQSPHDEDPQAVTYAKVKHSRPRREMASPPSPLSGEFLDTKDRQAEEDRQMDTEAAASEAPQDVTYAQLHSFTLRQKATEPPPSQEGASPAEPSVYATLAIH

SEQ ID NO: 147 - protein, 111 aa, synthetic construct
RHRHQSKHRTSAHFYRPAGAAGPEPKDQGLQKRASPVADIQEEILNAAVKDTQPKDGVEMDARAAASEAPQDVTYAQLHSLTLRREATEPPPSQEREPPAEPSIYAPLAIH

SEQ ID NO: 148 - protein, 179 aa, synthetic construct
KRKGRCSVPAFCSSQAEAPADTPEPTAGHTLYSVLSQGYEKLDTPLRPARQQPTPTSDSSSDSNLTTEEDEDRPEVHKPISGRYEVFDQVTQEGAGHDPAPEGQADYDPVTPYVTEVESVVGENTMYAQVFNLQGKTPVSQKEESSATIYCSIRKPQVVPPPQQNDLEIPESPTYENFT

SEQ ID NO: 149 - protein, 204 aa, synthetic construct
CSRAARGTIGARRTGQPLKEDPSAVPVFSVDYGELDFQWREKTPEPPVPCVPEQTEYATIVFPSGMGTSSPARRGSADGPRSAQPLRPEDGHCSWPLGGGGSGGGGSCSRAARGTIGARRTGQPLKEDPSAVPVFSVDYGELDFQWREKTPEPPVPCVPEQTEYATIVFPSGMGTSSPARRGSADGPRSAQPLRPEDGHCSWPL

SEQ ID NO: 150 - protein, 147 aa, synthetic construct
CSRAARGTIGARRTGQPLKEDPSAVPVFSVDYGELDFQWREKTPEPPVPCVPEQTEYATIDFQWREKTPEPPVPCVPEQVDYGELDFQWREKTPEPPVPCVPEQTEYATIVFPSGMGTSSPARRGSADGPRSAQPLRPEDGHCSWPL

SEQ ID NO: 151 - protein, 134 aa, synthetic construct
LRRHKHRPAPRLQPSRTSPQAPRARAWAPSQASQAALHVPYATINTSCRPATLDTAHPHGGPSWWASLPTHAAHRPQGPAAWASTPIPARGSFVSVENGLYAQAGERPPHTGPGLTLFPDPRGPRAMEGPLGVR

SEQ ID NO: 152 - protein, 122 aa, synthetic construct
RIREFRQTFRKIIRSHVLRQQEPFKAAGTSARVLAAHGSDGEQVSLRLNGHPPGVWANGSAPHPERRPNGYALGLVSGGSAQESQGNTGLPDVELLSHELKGVCPEPPGLDDPLAQDGAGVS

SEQ ID NO: 153 - protein, 5 aa, synthetic construct
GGGGS

SEQ ID NO: 154 - protein, 15 aa, synthetic construct
GGGGSGGGGSGGGGS

SEQ ID NO: 155 - protein, 21 aa, synthetic construct
GSGEGRGSLLTCGDVEENPGP

SEQ ID NO: 156 - protein, 25 aa, synthetic construct
GSGVKQTLNFDLLKLAGDVESNPGP

SEQ ID NO: 157 - protein, 22 aa, synthetic construct
GSGATNFSLLKQAGDVEENPGP

SEQ ID NO: 158 - protein, 23 aa, synthetic construct
GSGQCTNYALLKLAGDVESNPGP

SEQ ID NO: 159 - DNA, 567 bp, synthetic construct
  source 1..567: mol_type = other DNA
cccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaaacgtctaggccccccgaaccacggggacgtggttttcctttgaaaaacacgatgataatatg

SEQ ID NO: 160 - DNA, 414 bp, synthetic construct
  source 1..414: mol_type = other DNA
cccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaaacgtctaggccccccgaaccacggggacgtggttttcctttgaaaaacacgatgataatatg

SEQ ID NO: 161 - protein, 21 aa, synthetic construct
MALPVTALLLPLALLLHAARP

SEQ ID NO: 162 - protein, 22 aa, synthetic construct
MLLLVTSLLLCELPHPAFLLIP

SEQ ID NO: 163 - protein, 110 aa, synthetic construct
QSALTQPPSASGSPGQSVTISCTGTSSDVGGYKYVSWYQQHPGKAPKLMIYEVNKRPSGVPDRFSGSKSGNTASLTVSGLQAEDEADYYCSSYAGSNNWVFGGGTKLTVL

SEQ ID NO: 164 - protein, 119 aa, synthetic construct; identical to SEQ ID NO: 56

SEQ ID NO: 165 - protein, 113 aa, synthetic construct
DIVMTQSPDSLAVSLGERATISCKSSQSVLYSSNNKNYLAWYQQKPGQPPKLLIYWASTRESGVPDRFSGSGSGTDFTLTISSLQAEDVAVYYCQQYYGTPFTFGGGTKVEIK

SEQ ID NO: 166 - protein, 125 aa, synthetic construct
QVQLVQSGAEVKKPGASVKVSCKASGYTFTDYYLHWVRQAPGQGLEWMGWINPYTGGTNYAQKFQGRVTMTRDTSISTAYMELSGLTSDDTAVYYCARAGASYYDFWSGWVFDYWGQGTLVTVSS

SEQ ID NO: 167 - protein, 245 aa, synthetic construct
QVQLQQSGPELVKPGASVKMSCKASGYTFTSYHIQWVKQRPGQGLEWIGWIYPGDGSTQYNEKFKGKTTLTADKSSSTAYMLLSSLTSEDSAIYFCAREGTYYAMDYWGQGTSVTVSSGGGGSGGGGSGGGGSDVLMTQTPLSLPVSLGDQVSISCRSSQSIVHSNGNTYLEWYLQKPGQSPKLLIYKVSNRFSGVPDRFSGSGSGTDFTLKISRVEAEDLGVYYCFQGSHVPRTFGGGTKLEIK

SEQ ID NO: 168 - protein, 245 aa, synthetic construct
QVQLVQSGGGVVQPGGSLRVSCAASGVTLSDYGMHWVRQAPGKGLEWMAFIRNDGSDKYYADSVKGRFTISRDNSKKTVSLQMSSLRAEDTAVYYCAKNGESGPLDYWYFDLWGRGTLVTVSSGGGGSGGGGSGGGGSDIVMTQSPSFLSASVGDRVTITCRASHGINNYLAWYQQKPGKAPKLLIYAASTLQSGVPSRFSGSGSGTEFTLTISSLQPEDFATYYCQQYDSYPPTFGRGTKVEIK

SEQ ID NO: 169 - protein, 253 aa, synthetic construct
QVQLVQSGAEVKKPGASVKVSCKASGYTFTDYYLHWVRQAPGQGLEWMGWINPYTGGTNYAQKFQGRVTMTRDASISTVYMELSGLTSDDTAVHFCARAGASYYDFWSGWVFDYWGQGTLVTVSSGGGGSGGGGSGGGGSDIVMTQSPDSLAVSLGERATISCKSSQSVLYSSNNKNYLAWYQQKLGQPPKLLIYWASTRESGVPDRFSGSGSGTNFTLTISSLQAENVAVYYCQQYYGTPFTFGGGTKVEIK

SEQ ID NO: 170 - protein, 120 aa, synthetic construct [sequence omitted]

SEQ ID NO: 171 - protein, 107 aa, synthetic construct [sequence omitted]

SEQ ID NO: 172 - protein, 245 aa, synthetic construct
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSGSTSGSGKPGSGEGSTKGDIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIK

SEQ ID NO: 173 - protein, 119 aa, synthetic construct [sequence omitted]

SEQ ID NO: 174 - protein, 107 aa, synthetic construct [sequence omitted]

SEQ ID NO: 175 - protein, 244 aa, synthetic construct
DIQMTQSPSSLSASVGDRVTITCKASQDVSIGVAWYQQKPGKAPKLLIYSASYRYTGVPSRFSGSGSGTDFTLTISSLQPEDFATYYCQQYYIYPYTFGQGTKVEIKGSTSGSGKPGSGEGSTKGEVQLVESGGGLVQPGGSLRLSCAASGFTFTDYTMDWVRQAPGKGLEWVADVNPNSGGSIYNQRFKGRFTLSVDRSKNTLYLQMNSLRAEDTAVYYCARNLGPSFYFDYWGQGTLVTVSS

SEQ ID NO: 176 - protein, 119 aa, synthetic construct
QVQLQQSGPELKKPGETVKISCKASGYPFTNYGMNWVKQAPGQGLKWMGWINTSTGESTFADDFKGRFDFSLETSANTAYLQINNLKSEDMATYFCARWEVYHGYVPYWGQGTTVTVSS

SEQ ID NO: 177 - protein, 107 aa, synthetic construct
DIQLTQSHKFLSTSVGDRVSITCKASQDVYNAVAWYQQKPGQSPKLLIYSASSRYTGVPSRFTGSGSGPDFTFTISSVQAEDLAVYFCQQHFRTPFTFGSGTKLEIK

SEQ ID NO: 178 - protein, 120 aa, synthetic construct
EVQLQQSGPEVVKTGASVKISCKASGYSFTGYFINWVKKNSGKSPEWIGHISSSYATSTYNQKFKNKAAFTVDTSSSTAFMQLNSLTSEDSAVYYCVRSGNYEEYAMDYWGQGTSVTVSS

SEQ ID NO: 179 - protein, 113 aa, synthetic construct
DIVLTQTPSSLPVSVGEKVTMTCKSSQTLLYSNNQKNYLAWYQQKPGQSPKLLISWAFTRKSGVPDRFTGSGSGTDFTLTIGSVKAEDLAVYYCQQYSNYPWTFGGGTKLEIK

SEQ ID NO: 180 - protein, 121 aa, synthetic construct
QVQLVESGGGVVQPGRSLRLSCAASGFTFSSYGMHWVRQAPGKGLEWVAVIWYDGSNKYYADSVKGRFTISRDNSKNTLYLQMNSLRAEDTAVYYCAKEAPYYAKDYMDVWGKGTTVTVSS

SEQ ID NO: 181 - protein, 107 aa, synthetic construct
EIVLTQSPGTLSLSPGERATLSCRASQSVSSDYLAWYQQKPGQAPRLLIYGASSRATGIPDRFSGSGSGTDFTLTISRLEPEDFAVYYCQQYVSYWTFGGGTKVEIK

SEQ ID NO: 182 - protein, 121 aa, synthetic construct
QVQLVESGGGVVQPGRSLRLSCAASGFTFSSYGMHWVRQAPGKGLEWVAGIWWDGSNEKYADSVKGRFTISRDNSKNTLYLQMNSLRAEDTAVYYCAKEAPYYAKDYMDVWGKGTTVTVSS

SEQ ID NO: 183 - protein, 107 aa, synthetic construct
EIVLTQSPGTLSLSPGERATLSCRASQSVSSDYLAWYQQKPGQAPRLLIYGASRRATGIPDRFSGSGSGTDFTLTISRLEPEDFAVYYCQQYVSYWTFGGGTKVEIK

SEQ ID NO: 184 - protein, 117 aa, synthetic construct
EVQLVESGGGLVQPGGSLRLSCAASGFTFSSYSMNWVRQAPGKGLEWVSYISSSSSTIYYADSVKGRFTISRDNAKNSLYLQMNSLRAEDTAVYYCARGGHGYFDLWGRGTLVTVSS

SEQ ID NO: 185 - protein, 108 aa, synthetic construct
EIVLTQSPGTLSLSPGERATLSCRASQSVSSSYLAWYQQKPGQAPRLLIYGASSRATGIPDRFSGSGSGTDFTLTISRLEPEDFAVYYCQQYHHSPLTFGGGTKVEIK

SEQ ID NO: 186 - protein, 117 aa, synthetic construct
QVQLQQSGAELVRPGTSVKLSCKASDYTFTSYWMNWVKQRPGQGLEWIGMIDPSDSETQYNQMFKDKAALTVDKSSNTAYMQLSSLTSEDSAVYYCAKGGASGDWYFDVWGQGTTVT

SEQ ID NO: 187 - protein, 105 aa, synthetic construct
DIQLTQSPSSLSASLGGEVTITCKASQDIKKYIAWYQHKPGKSPRLLIHYTSVLQPGIPSRFSGSGSGRDYSFSIHNLEPEDIATYYCLHYDYLYTFGGGTKLEI

SEQ ID NO: 188 - protein, 270 aa, synthetic construct
MLLLVTSLLLCELPHPAFLLIPDYKDDDDKQVQLQQSGAELVRPGTSVKLSCKASDYTFTSYWMNWVKQRPGQGLEWIGMIDPSDSETQYNQMFKDKAALTVDKSSNTAYMQLSSLTSEDSAVYYCAKGGASGDWYFDVWGQGTTVTGSTSGSGKPGSGEGSTKGDIQLTQSPSSLSASLGGEVTITCKASQDIKKYIAWYQHKPGKSPRLLIHYTSVLQPGIPSRFSGSGSGRDYSFSIHNLEPEDIATYYCLHYDYLYTFGGGTKLEI

SEQ ID NO: 189 - protein, 118 aa, synthetic construct
QVQLKQSGPGLVQPSQSLSITCTVSGFSLTNYGVHWVRQSPGKGLEWLGVIWSGGNTDYNTPFTSRLSINKDNSKSQVFFKMNSLQSNDTAIYYCARALTYYDYEFAYWGQGTLVTVS

SEQ ID NO: 190 - protein, 107 aa, synthetic construct
DILLTQSPVILSVSPGERVSFSCRASQSIGTNIHWYQQRTNGSPRLLIKYASESISGIPSRFSGSGSGTDFTLSINSVESEDIADYYCQQNNNWPTTFGAGTKLELK

SEQ ID NO: 191 - protein, 243 aa, synthetic construct
QVQLKQSGPGLVQPSQSLSITCTVSGFSLTNYGVHWVRQSPGKGLEWLGVIWSGGNTDYNTPFTSRLSINKDNSKSQVFFKMNSLQSNDTAIYYCARALTYYDYEFAYWGQGTLVTVSGSTSGSGKPGSGEGSTKGDILLTQSPVILSVSPGERVSFSCRASQSIGTNIHWYQQRTNGSPRLLIKYASESISGIPSRFSGSGSGTDFTLSINSVESEDIADYYCQQNNNWPTTFGAGTKLELK

SEQ ID NO: 192 - protein, 119 aa, synthetic construct [sequence omitted]

SEQ ID NO: 193 - protein, 107 aa, synthetic construct [sequence omitted]

SEQ ID NO: 194 - protein, 244 aa, synthetic construct
DIQMTQSPSSLSASVGDRVTITCQASQDISNYLNWYQQKPGKAPKLLIYDASNLETGVPSRFSGSGSGTDFTFTISSLQPEDIATYFCQHFDHLPLAFGGGTKVEIKGSTSGSGKPGSGEGSTKGQVQLQESGPGLVKPSETLSLTCTVSGGSVSSGDYYWTWIRQSPGKGLEWIGHIYYSGNTNYNPSLKSRLTISIDTSKTQFSLKLSSVTAADTAIYYCVRDRVTGAFDIWGQGTMVTVSS

SEQ ID NO: 195 - protein, 120 aa, synthetic construct [sequence omitted]

SEQ ID NO: 196 - protein, 106 aa, synthetic construct [sequence omitted]

SEQ ID NO: 197 - protein, 123 aa, synthetic construct
QVQLQQSGAEVKKPGSSVKVSCKASGYTFTNYYIYWVRQAPGQGLEWIGGINPTSGGSNFNEKFKTRVTITVDESTNTAYMELSSLRSEDTAFYFCARQGLWFDSDGRGFDFWGQGSTVTVSS

SEQ ID NO: 198 - protein, 112 aa, synthetic construct [sequence omitted]

SEQ ID NO: 199 - protein, 121 aa, synthetic construct [sequence omitted]

SEQ ID NO: 200 - protein, 107 aa, synthetic construct [sequence omitted]

SEQ ID NO: 201 - protein, 120 aa, synthetic construct
QVNLLQSGAALVKPGASVKLSCKGSGFTFTDYKIHWVKQSHGKSLEWIGYFNPNSGYSTYNEKFKSKATLTADKSTDTAYMELTSLTSEDSATYYCTRLSPGGYYVMDAWGQGASVTVSS

SEQ ID NO: 202 - protein, 106 aa, synthetic construct
DIQMTQSPSFLSASVGDRVTINCKASQNINNYLNWYQQKLGEAPKRLIYNTNNLQTGIPSRFSGSGSGTDYTLTISSLQPEDFATYFCLQHNSFPTFGAGTKLELK

SEQ ID NO: 203 - protein, 266 aa, synthetic construct
MLLLVTSLLLCELPHPAFLLIPDIQMTQSPSFLSASVGDRVTINCKASQNINNYLNWYQQKLGEAPKRLIYNTNNLQTGIPSRFSGSGSGTDYTLTISSLQPEDFATYFCLQHNSFPTFGAGTKLELKGSTSGSGKPGSGEGSTKGQVNLLQSGAALVKPGASVKLSCKGSGFTFTDYKIHWVKQSHGKSLEWIGYFNPNSGYSTYNEKFKSKATLTADKSTDTAYMELTSLTSEDSATYYCTRLSPGGYYVMDAWGQGASVTVSS

SEQ ID NO: 204 - protein, 121 aa, synthetic construct [sequence omitted]

SEQ ID NO: 205 - protein, 106 aa, synthetic construct [sequence omitted]

SEQ ID NO: 206 - protein, 126 aa, synthetic construct
EVQLVQSGAEVKKPGSSVKVSCKASGGTFSSYAIGWVRQAPGQGLEWMGGIIPIFGIANYAQKFQGRVTITADESTSSAYMELSSLRSEDTAVYYCAREEGPYCSSTSCYAAFDIWGQGTLVTLSS

SEQ ID NO: 207 - protein, 108 aa, synthetic construct
QSVLTQDPAVSVALGQTVKITCQGDSLRSYFASWYQQKPGQAPTLVMYARNDRPAGVPDRFSGSKSGTSASLSAISGLQPEDEAYYCAAWDDSLNGYLFGAGTKLTVL

SEQ ID NO: 208 - protein, 125 aa, synthetic construct [sequence omitted]

SEQ ID NO: 209 - protein, 107 aa, synthetic construct [sequence omitted]

SEQ ID NO: 210 - protein, 121 aa, synthetic construct
QVQLVQSGAEVKKPGSSVKVSCKASGGTFSSYAISWVRQAPGQGLEWMGSIIPIFGTVNYAQKFQGRVTITADESTSTAYMELSSLRSEDTAVYYCARDPSVNLYWYFDLWGRGTLVTVSS

SEQ ID NO: 211 - protein, 107 aa, synthetic construct
DIQMTQSPSTLSASVGDRVTITCRASQSISSWWAWYQQKPGKAPKLLIYDASSLESGVPSRFSGSGSGTEFTLTISSLQPDDFATYYCQQYHAHPTTFGGGTKVEIK

SEQ ID NO: 212 - protein, 119 aa, synthetic construct
QVQLVQSGAEVKKPGSSVKVSCKASGGTFGSYAISWVRQAPGQGLEWMGSIIPIFGAANPAQKSQGRVTITADESTSTAYMELSSLRSEDTAVYYCAKMGRGKVAFDIWGQGTMVTVSS

SEQ ID NO: 213 - protein, 113 aa, synthetic construct
DIVMTQSPDSLAVSLGERATINCKSSQSVLYSPNNKNYLAWYQQKPGQPPKLLIYWASTRESGVPDRFSGSGSGTDFTLTISSLQAEDVAVYYCQQYYGSPITFGGGTKVEIK

SEQ ID NO: 214 - protein, 123 aa, synthetic construct
QVQLVQSGAEVKKPGASVKVSCKASGYAFTSYGINWVRQAPGQGLEWMGWISAYNGNTYYAQKLRGRVTMTTDTSTSTAYMELRSLRSDDTAVYYCARDLGGYGSGSVPFDPWGQGTLVTVSS

SEQ ID NO: 215 - protein, 108 aa, synthetic construct
EIVMTQSPATLSVSPGERATLSCRASQSVSSNLAWYQQKPGQAPRLLIYGASTRATGIPARFSGSGSGTEFTLTISSLQSEDFAVYYCQDYRTWPRRVFGGGTKVEIK

SEQ ID NO: 216 - protein, 129 aa, synthetic construct
QVQLQESGGGLVQAGGSLLLSCAASGRTFSSYAMGWFRQAPGKEREFVAAINWSGGSTSYADSVKGRFTISRDNTKNTVYLQMNSLKPEDTAAFYCAATYNPYSRDHYFPRMTTEYDYWGQGTQVTVSS

SEQ ID NO: 217 - protein, 119 aa, synthetic construct
QVQLVQSGAEVKKPGASVKVSCKASGYSFTGYTMNWVRQAPGQGLEWMGLITPYNGASSYNQKFRGKATMTVDTSTSTVYMELSSLRSEDTAVYYCARGGYDGRGFDYWGQGTLVTVSS

SEQ ID NO: 218 - protein, 106 aa, synthetic construct
DIQMTQSPSSLSASVGDRVTITCSASSSVSYMHWYQQKSGKAPKLLIYDTSKLASGVPSRFSGSGSGTDFTLTISSLQPEDFATYYCQQWSKHPLTFGQGTKLEIK

SEQ ID NO: 219 - protein, 123 aa, synthetic construct
QVQLQQSGPGLVTPSQTLSLTCAISGDSVSSNSATWNWIRQSPSRGLEWLGRTYYRSKWYNDYAVSVKSRMSINPDTSKNQFSLQLNSVTPEDTAVYYCARGMMTYYYGMDVWGQGTTVTVSS

SEQ ID NO: 220 - protein, 115 aa, synthetic construct
QPVLTQSSSLSASPGASASLTCTLRSGINVGPYRIYWYQQKPGSPPQYLLNYKSDSDKQQGSGVPSRFSGSKDASANAGVLLISGLRSEDEADYYCMIWHSSAAVFGGGTQLTVL

SEQ ID NO: 221 - protein, 278 aa, synthetic construct
MLLLVTSLLLCELPHPAFLLIPQPVLTQSSSLSASPGASASLTCTLRSGINVGPYRIYWYQQKPGSPPQYLLNYKSDSDKQQGSGVPSRFSGSKDASANAGVLLISGLRSEDEADYYCMIWHSSAAVFGGGTQLTVLGSTSGSGKPGSGEGSTKGQVQLQQSGPGLVTPSQTLSLTCAISGDSVSSNSATWNWIRQSPSRGLEWLGRTYYRSKWYNDYAVSVKSRMSINPDTSKNQFSLQLNSVTPEDTAVYYCARGMMTYYYGMDVWGQGTTVTVSS

SEQ ID NO: 222 - protein, 119 aa, synthetic construct
QVQLQQSGPELEKPGASVKISCKASGYSFTGYTMNWVKQSHGKSLEWIGLITPYNGASSYNQKFRGKATLTVDKSSSTAYMDLLSLTSEDSAVYFCARGGYDGRGFDYWGSGTPVTVSS

SEQ ID NO: 223 - protein, 106 aa, synthetic construct
DIELTQSPAIMSASPGEKVTMTCSASSSVSYMHWYQQKSGTSPKRWIYDTSKLASGVPGRFSGSGSGNSYSLTISSVEAEDDATYYCQQWSKHPLTFGSGTKVEIK

SEQ ID NO: 224 - protein, 259 aa, synthetic construct
MLLLVTSLLLCELPHPDIELTQSPAIMSASPGEKVTMTCSASSSVSYMHWYQQKSGTSPKRWIYDTSKLASGVPGRFSGSGSGNSYSLTISSVEAEDDATYYCQQWSKHPLTFGSGTKVEIKGSTSGSGKPGSGEGSTKGQVQLQQSGPELEKPGASVKISCKASGYSFTGYTMNWVKQSHGKSLEWIGLITPYNGASSYNQKFRGKATLTVDKSSSTAYMDLLSLTSEDSAVYFCARGGYDGRGFDYWGSGTPVTVSS

SEQ ID NO: 225 - protein, 114 aa, synthetic construct
QVQLVQSGGGLVQPGGSLRLSCAASDFDFAAYEMSWVRQSAPGQGLEWVAIISHDGIDKYYTDSVKGRFTISRDNSKNTLYLQMNTLRAEDTATYYCLRLGAVGQGTLVTVSSS

SEQ ID NO: 226 - protein, 120 aa, synthetic construct
QVQLVQSGGGLVQPGGSLRLSCAASDFAFDDYEMSWVRQAPGKALEWIGDINHSGTTIYNPSLKSRVTISRDNSKNTLYLQMNTLRAEDTAIYYCARPHYGDYSDAFDIWGQGTMVTVSS

SEQ ID NO: 227 - protein, 118 aa, synthetic construct
EVQLQQSGTVLARPGASVKMSCKASGYSFTNYRMNWVKQRPGQGLEWIGGIYPGNRDTTYNQKFKDKAKLTAVTSANTAYMELSSLTNEDSAVYYCTRGVIGIYFDYWGQGTTLTVSS

SEQ ID NO: 228 - protein, 111 aa, synthetic construct
DIVMTQSPASLAVSLGQRATISCKASQSVDYDGDSYMNWYQQKPGQPPKLLIYAASNLESGIPARFSGSGSGTDFTLNIHPVEEEDAATYYCQQNNEAPLTFGAGTKLELK

SEQ ID NO: 229 - protein, 269 aa, synthetic construct
MLLLVTSLLLCELPHPAFLLIPDIVMTQSPASLAVSLGQRATISCKASQSVDYDGDSYMNWYQQKPGQPPKLLIYAASNLESGIPARFSGSGSGTDFTLNIHPVEEEDAATYYCQQNNEAPLTFGAGTKLELKGSTSGSGKPGSGEGSTKGEVQLQQSGTVLARPGASVKMSCKASGYSFTNYRMNWVKQRPGQGLEWIGGIYPGNRDTTYNQKFKDKAKLTAVTSANTAYMELSSLTNEDSAVYYCTRGVIGIYFDYWGQGTTLTVSS

SEQ ID NO: 230 - protein, 118 aa, synthetic construct
QVQLQQSGTVLARPGASVKMSCKASGYSFTNYRMYWVKQRPGQGLEWIGAIYPGNSDTTYKQKFKGKAKLTAVTSASTAYMELSSLTNEDSAVYYCTRGIRGSYFDVWGAGTTVTVSS

SEQ ID NO: 231 - protein, 111 aa, synthetic construct
DIVMTQSPASLAVSLGQRATISCKASQSVDYDGDSYMNWYQQKPGQPPKLLIYAASNLESGIPARFSGSGSGTDFTLNIHPVEEEDAATYYCQQSNEDPYTFGGGTKLEIK

SEQ ID NO: 232 - protein, 269 aa, synthetic construct
MLLLVTSLLLCELPHPAFLLIPDIVMTQSPASLAVSLGQRATISCKASQSVDYDGDSYMNWYQQKPGQPPKLLIYAASNLESGIPARFSGSGSGTDFTLNIHPVEEEDAATYYCQQSNEDPYTFGGGTKLEIKGSTSGSGKPGSGEGSTKGQVQLQQSGTVLARPGASVKMSCKASGYSFTNYRMYWVKQRPGQGLEWIGAIYPGNSDTTYKQKFKGKAKLTAVTSASTAYMELSSLTNEDSAVYYCTRGIRGSYFDVWGAGTTVTVSS

SEQ ID NO: 233 - protein, 42 aa, synthetic construct
KRGRKKLLYIFKQPFMRPVQTTQEEDGCSCRFPEEEEGGCEL

SEQ ID NO: 234 - protein, 39 aa, synthetic construct
RSKRSRLLHSDYMNMTPRRPGPTRKHYQPYAPPRDFAAY

SEQ ID NO: 235 - protein, 112 aa, synthetic construct
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 236 - protein, 112 aa, synthetic construct; identical to SEQ ID NO: 235

SEQ ID NO: 237 - protein, 112 aa, synthetic construct
RVKFSRSADAPAYQQGQNQLFNELNLGRREEYDVLDKRRGRDPEMGGKPRRKNPQEGLYNELQKDKMAEAFSEIGMKGERRRGKGHDGLFQGLSTATKDTYDALHMQALPPR

SEQ ID NO: 238 - protein, 112 aa, synthetic construct
RVKFSRSADAPAYQQGQNQLFNELNLGRREEFDVLDKRRGRDPEMGGKPRRKNPQEGLYNELQKDKMAEAFSEIGMKGERRRGKGHDGLFQGLSTATKDTYDALHMQALPPR

SEQ ID NO: 239 - protein, 112 aa, synthetic construct
RVKFSRSADAPAYQQGQNQLFNELNLGRREEFDVLDKRRGRDPEMGGKPRRKNPQEGLFNELQKDKMAEAYSEIGMKGERRRGKGHDGLYQGLSTATKDTFDALHMQALPPR

SEQ ID NO: 240 - DNA, 55 bp, synthetic construct
  source 1..55: mol_type = other DNA
ggattacatcgccctgaaagttcaagagactttcagggcgatgtaatcctttttt

SEQ ID NO: 241 - DNA, 51 bp, synthetic construct
  source 1..51: mol_type = other DNA
cacctgccatgtgcagcatgatttgtgtagtcatgctgcacatggcaggtg

SEQ ID NO: 242 - DNA, 47 bp, synthetic construct
  source 1..47: mol_type = other DNA
gaatggagagagaattgaattcaagagattcaattctctctccattc

SEQ ID NO: 243 - protein, 72 aa, synthetic construct
CSRAARGTIGARRTGQPLKEDPSAVPVFSVDYGELVFPSGMGTSSPARRGSADGPRSAQPLRPEDGHCSWPL

SEQ ID NO: 244 - protein, 97 aa, synthetic construct
CSRAARGTIGARRTGQPLKEDPSAVPVFSVDYGELDFQWREKTPEPPVPCVPEQVDYGELVFPSGMGTSSPARRGSADGPRSAQPLRPEDGHCSWPL

SEQ ID NO: 245 - protein, 122 aa, synthetic construct
CSRAARGTIGARRTGQPLKEDPSAVPVFSVDYGELDFQWREKTPEPPVPCVPEQVDYGELDFQWREKTPEPPVPCVPEQVDYGELVFPSGMGTSSPARRGSADGPRSAQPLRPEDGHCSWPL

SEQ ID NO: 246 - protein, 147 aa, synthetic construct
CSRAARGTIGARRTGQPLKEDPSAVPVFSVDYGELDFQWREKTPEPPVPCVPEQVDYGELDFQWREKTPEPPVPCVPEQVDYGELDFQWREKTPEPPVPCVPEQVDYGELVFPSGMGTSSPARRGSADGPRSAQPLRPEDGHCSWPL

SEQ ID NO: 247 - protein, 172 aa, synthetic construct
CSRAARGTIGARRTGQPLKEDPSAVPVFSVDYGELDFQWREKTPEPPVPCVPEQVDYGELDFQWREKTPEPPVPCVPEQVDYGELDFQWREKTPEPPVPCVPEQVDYGELDFQWREKTPEPPVPCVPEQVDYGELVFPSGMGTSSPARRGSADGPRSAQPLRPEDGHCSWPL

SEQ ID NO: 248 - protein, 72 aa, synthetic construct
CSRAARGTIGARRTGQPLKEDPSAVPVFSTEYATIVFPSGMGTSSPARRGSADGPRSAQPLRPEDGHCSWPL

SEQ ID NO: 249 - protein, 97 aa, synthetic construct
CSRAARGTIGARRTGQPLKEDPSAVPVFSTEYATIDFQWREKTPEPPVPCVPEQTEYATIVFPSGMGTSSPARRGSADGPRSAQPLRPEDGHCSWPL

SEQ ID NO: 250 - protein, 122 aa, synthetic construct
CSRAARGTIGARRTGQPLKEDPSAVPVFSTEYATIDFQWREKTPEPPVPCVPEQTEYATIDFQWREKTPEPPVPCVPEQTEYATIVFPSGMGTSSPARRGSADGPRSAQPLRPEDGHCSWPL

SEQ ID NO: 251 - protein, 147 aa, synthetic construct
CSRAARGTIGARRTGQPLKEDPSAVPVFSTEYATIDFQWREKTPEPPVPCVPEQTEYATIDFQWREKTPEPPVPCVPEQTEYATIDFQWREKTPEPPVPCVPEQTEYATIVFPSGMGTSSPARRGSADGPRSAQPLRPEDGHCSWPL

SEQ ID NO: 252 - protein, 172 aa, synthetic construct
CSRAARGTIGARRTGQPLKEDPSAVPVFSTEYATIDFQWREKTPEPPVPCVPEQTEYATIDFQWREKTPEPPVPCVPEQTEYATIDFQWREKTPEPPVPCVPEQTEYATIDFQWREKTPEPPVPCVPEQTEYATIVFPSGMGTSSPARRGSADGPRSAQPLRPEDGHCSWPL

SEQ ID NO: 253 - protein, 204 aa, synthetic construct; identical to SEQ ID NO: 149

SEQ ID NO: 254 - protein, 147 aa, synthetic construct; identical to SEQ ID NO: 150

SEQ ID NO: 255 - protein, 480 aa, synthetic construct
MALPVTALLLPLALLLHAARPQSALTQPPSASGSPGQSVTISCTGTSSDVGGYKYVSWYQHHPDKAPKLMIYEVNKRPSGVPDRFSGSKSDNTASLTVSGLQAEDEADYYCSSYAGSNNWVFGGGTKLTVLGGGGSGGGGSGGGGSQVQLVESGGGVVQPGGSLRLSCAASGFTFSTYGMHWVRQAPGKGLEWVASISYDGSNKYYADSGQGRFTISRDTSKNSLYLQMNSLRAEDTAVYYCAIGIYGAYSFDYWGQGTLVTVSSTERRAEVPTAHPSPSPRPAGQFQTLVVGVVGGLLGSLVLLVWVLAVILRHRRQGKHWTSTQRKADFQHPAGAVGPEPTDRGLQWRSSPAADAQEENLYAAVKHTQPEDGVEMDTRSPHDEDPQAVTYAEVKHSRPRREMASPPSPLSGEFLDTKDRQAEEDRQMDTEAAASEAPQDVTYAQLHSLTLRREATEPPPSQEGPSPAVPSIYATLAIH

SEQ ID NO: 256 - protein, 480 aa, synthetic construct
MALPVTALLLPLALLLHAARPQSALTQPPSASGSPGQSVTISCTGTSSDVGGYKYVSWYQQHPGKAPKLMIYEVNKRPSGVPDRFSGSKSGNTASLTVSGLQAEDEADYYCSSYAGSNNWVFGGGTKLTVLGGGGSGGGGSGGGGSQVQLVESGGGVVQPGGSLRLSCAASGFTFSTYGMHWVRQAPGKGLEWVASISYDGSNKYYADSGQGRFTISRDTSKNSLYLQMNSLRAEDTAVYYCAIGIYGAYSFDYWGQGTLVTVSSTERRAEVPTAHPSPSPRPAGQFQTLVVGVVGGLLGSLVLLVWVLAVILRHRRQGKHWTSTQRKADFQHPAGAVGPEPTDRGLQWRSSPAADAQEENLYAAVKHTQPEDGVEMDTRSPHDEDPQAVTYAEVKHSRPRREMASPPSPLSGEFLDTKDRQAEEDRQMDTEAAASEAPQDVTYAQLHSLTLRREATEPPPSQEGPSPAVPSIYATLAIH

SEQ ID NO: 257 - protein, 489 aa, synthetic construct
MALPVTALLLPLALLLHAARPDIVMTQSPDSLAVSLGERATISCKSSQSVLYSSNNKNYLAWYQQKLGQPPKLLIYWASTRESGVPDRFSGSGSGTNFTLTISSLQAENVAVYYCQQYYGTPFTFGGGTKVEIKGGGGSGGGGSGGGGSQVQLVQSGAEVKKPGASVKVSCKASGYTFTDYYLHWVRQAPGQGLEWMGWINPYTGGTNYAQKFQGRVTMTRDASISTVYMELSGLTSDDTAVHFCARAGASYYDFWSGWVFDYWGQGTLVTVSSTERRAEVPTAHPSPSPRPAGQFQTLVVGVVGGLLGSLVLLVWVLAVILRHRRQGKHWTSTQRKADFQHPAGAVGPEPTDRGLQWRSSPAADAQEENLYAAVKHTQPEDGVEMDTRSPHDEDPQAVTYAEVKHSRPRREMASPPSPLSGEFLDTKDRQAEEDRQMDTEAAASEAPQDVTYAQLHSLTLRREATEPPPSQEGPSPAVPSIYATLAIH

SEQ ID NO: 258 - protein, 493 aa, synthetic construct
MALPVTALLLPLALLLHAARPDIVMTQSPDSLAVSLGERATISCKSSQSVLYSSNNKNYLAWYQQKPGQPPKLLIYWASTRESGVPDRFSGSGSGTDFTLTISSLQAEDVAVYYCQQYYGTPFTFGGGTKVEIKGGGGSGGGGSGGGGSQVQLVQSGAEVKKPGASVKVSCKASGYTFTDYYLHWVRQAPGQGLEWMGWINPYTGGTNYAQKFQGRVTMTRDTSISTAYMELSGLTSDDTAVYYCARAGASYYDFWSGWVFDYWGQGTLVTVSSGPTSTSGPEDQPLTPTGSDPQSGLGRHLGVVIGILVAVILLLLLLLLLFLILRHRRQGKHWTSTQRKADFQHPAGAVGPEPTDRGLQWRSSPAADAQEENLYAAVKHTQPEDGVEMDTRSPHDEDPQAVTYAEVKHSRPRREMASPPSPLSGEFLDTKDRQAEEDRQMDTEAAASEAPQDVTYAQLHSLTLRREATEPPPSQEGPSPAVPSIYATLAIH

SEQ ID NO: 259 - protein, 507 aa, synthetic construct
MALPVTALLLPLALLLHAARPQVQLVQSGAEVKKPGSSVKVSCKASGYTFTSYHIQWVRQAPGQGLEWMGWIYPGDGSTQYNEKFKGRTTITADKSTSTAYMELSSLRSEDTAVYYCAREGTYYAMDYWGQGTLVTVSSGGGGSGGGGSGGGGSDVVMTQTPLSLSVTPGQPASISCRSSQSIVHSNGNTYLEWYLQKPGQSPQLLIYKVSNRFSGVPDRFSGSGSGTDFTLKISRVEAEDVGVYYCFQGSHVPRTFGGGTKVEIKHPSDPLELVVSGPSGGPSSPTTGPTSTSGPEDQPLTPTGSDPQSGLGRHLGVVIGILVAVILLLLLLLLLFLILRHRRQGKHWTSTQRKADFQHPAGAVGPEPTDRGLQWRSSPAADAQEENLYAAVKHTQPEDGVEMDTRSPHDEDPQAVTYAEVKHSRPRREMASPPSPLSGEFLDTKDRQAEEDRQMDTEAAASEAPQDVTYAQLHSLTLRREATEPPPSQEGPSPAVPSIYATLAIH

SEQ ID NO: 260 - protein, 507 aa, synthetic construct
MALPVTALLLPLALLLHAARPQVQLVQSGAEVKKPGASVKVSCKASGYTFTSYHIQWVRQAPGQRLEWIGWIYPGDGSTQYNEKFKGRVTITRDTSASTAYMELSSLRSEDTAVYYCAREGTYYAMDYWGQGTLVTVSSGGGGSGGGGSGGGGSDVVMTQTPLSLSVTPGQPASISCRSSQSIVHSNGNTYLEWYLQKPGQSPQLLIYKVSNRFSGVPDRFSGSGSGTDFTLKISRVEAEDVGVYYCFQGSHVPRTFGGGTKVEIKHPSDPLELVVSGPSGGPSSPTTGPTSTSGPEDQPLTPTGSDPQSGLGRHLGVVIGILVAVILLLLLLLLLFLILRHRRQGKHWTSTQRKADFQHPAGAVGPEPTDRGLQWRSSPAADAQEENLYAAVKHTQPEDGVEMDTRSPHDEDPQAVTYAEVKHSRPRREMASPPSPLSGEFLDTKDRQAEEDRQMDTEAAASEAPQDVTYAQLHSLTLRREATEPPPSQEGPSPAVPSIYATLAIH

SEQ ID NO: 261 - protein, 395 aa, synthetic construct
MALPVTALLLPLALLLHAARPEQKLISEEDLQVQLQQSGPELVKPGASVKMSCKASGYTFTSYHIQWVKQRPGQGLEWIGWIYPGDGSTQYNEKFKGKTTLTADKSSSTAYMLLSSLTSEDSAIYFCAREGTYYAMDYWGQGTSVTVSSGGGGSGGGGSGGGGSDVLMTQTPLSLPVSLGDQVSISCRSSQSIVHSNGNTYLEWYLQKPGQSPKLLIYKVSNRFSGVPDRFSGSGSGTDFTLKISRVEAEDLGVYYCFQGSHVPRTFGGGTKLEIKTERRAEVPTAHPSPSPRPAGQFQTLVVGVVGGLLGSLVLLVWVLAVICSRAARGTIGARRTGQPLKEDPSAVPVFSVDYGELVFPSGMGTSSPARRGSADGPRSAQPLRPEDGHCSWPL

SEQ ID NO: 262 - protein, 420 aa, synthetic construct
MALPVTALLLPLALLLHAARPEQKLISEEDLQVQLQQSGPELVKPGASVKMSCKASGYTFTSYHIQWVKQRPGQGLEWIGWIYPGDGSTQYNEKFKGKTTLTADKSSSTAYMLLSSLTSEDSAIYFCAREGTYYAMDYWGQGTSVTVSSGGGGSGGGGSGGGGSDVLMTQTPLSLPVSLGDQVSISCRSSQSIVHSNGNTYLEWYLQKPGQSPKLLIYKVSNRFSGVPDRFSGSGSGTDFTLKISRVEAEDLGVYYCFQGSHVPRTFGGGTKLEIKTERRAEVPTAHPSPSPRPAGQFQTLVVGVVGGLLGSLVLLVWVLAVICSRAARGTIGARRTGQPLKEDPSAVPVFSVDYGELDFQWREKTPEPPVPCVPEQVDYGELVFPSGMGTSSPARRGSADGPRSAQPLRPEDGHCSWPL

SEQ ID NO: 263 - protein, 445 aa, synthetic construct
MALPVTALLLPLALLLHAARPEQKLISEEDLQVQLQQSGPELVKPGASVKMSCKASGYTFTSYHIQWVKQRPGQGLEWIGWIYPGDGSTQYNEKFKGKTTLTADKSSSTAYMLLSSLTSEDSAIYFCAREGTYYAMDYWGQGTSVTVSSGGGGSGGGGSGGGGSDVLMTQTPLSLPVSLGDQVSISCRSSQSIVHSNGNTYLEWYLQKPGQSPKLLIYKVSNRFSGVPDRFSGSGSGTDFTLKISRVEAEDLGVYYCFQGSHVPRTFGGGTKLEIKTERRAEVPTAHPSPSPRPAGQFQTLVVGVVGGLLGSLVLLVWVLAVICSRAARGTIGARRTGQPLKEDPSAVPVFSVDYGELDFQWREKTPEPPVPCVPEQVDYGELDFQWREKTPEPPVPCVPEQVDYGELVFPSGMGTSSPARRGSADGPRSAQPLRPEDGHCSWPL

SEQ ID NO: 264 - protein, 470 aa, synthetic construct
MALPVTALLLPLALLLHAARPEQKLISEEDLQVQLQQSGPELVKPGASVKMSCKASGYTFTSYHIQWVKQRPGQGLEWIGWIYPGDGSTQYNEKFKGKTTLTADKSSSTAYMLLSSLTSEDSAIYFCAREGTYYAMDYWGQGTSVTVSSGGGGSGGGGSGGGGSDVLMTQTPLSLPVSLGDQVSISCRSSQSIVHSNGNTYLEWYLQKPGQSPKLLIYKVSNRFSGVPDRFSGSGSGTDFTLKISRVEAEDLGVYYCFQGSHVPRTFGGGTKLEIKTERRAEVPTAHPSPSPRPAGQFQTLVVGVVGGLLGSLVLLVWVLAVICSRAARGTIGARRTGQPLKEDPSAVPVFSVDYGELDFQWREKTPEPPVPCVPEQVDYGELDFQWREKTPEPPVPCVPEQVDYGELDFQWREKTPEPPVPCVPEQVDYGELVFPSGMGTSSPARRGSADGPRSAQPLRPEDGHCSWPL

SEQ ID NO: 265 - protein, 495 aa, synthetic construct
MALPVTALLLPLALLLHAARPEQKLISEEDLQVQLQQSGPELVKPGASVKMSCKASGYTFTSYHIQWVKQRPGQGLEWIGWIYPGDGSTQYNEKFKGKTTLTADKSSSTAYMLLSSLTSEDSAIYFCAREGTYYAMDYWGQGTSVTVSSGGGGSGGGGSGGGGSDVLMTQTPLSLPVSLGDQVSISCRSSQSIVHSNGNTYLEWYLQKPGQSPKLLIYKVSNRFSGVPDRFSGSGSGTDFTLKISRVEAEDLGVYYCFQGSHVPRTFGGGTKLEIKTERRAEVPTAHPSPSPRPAGQFQTLVVGVVGGLLGSLVLLVWVLAVICSRAARGTIGARRTGQPLKEDPSAVPVFSVDYGELDFQWREKTPEPPVPCVPEQVDYGELDFQWREKTPEPPVPCVPEQVDYGELDFQWREKTPEPPVPCVPEQVDYGELDFQWREKTPEPPVPCVPEQVDYGELVFPSGMGTSSPARRGSADGPRSAQPLRPEDGHCSWPL

SEQ ID NO: 266 - protein, 395 aa, synthetic construct
MALPVTALLLPLALLLHAARPEQKLISEEDLQVQLQQSGPELVKPGASVKMSCKASGYTFTSYHIQWVKQRPGQGLEWIGWIYPGDGSTQYNEKFKGKTTLTADKSSSTAYMLLSSLTSEDSAIYFCAREGTYYAMDYWGQGTSVTVSSGGGGSGGGGSGGGGSDVLMTQTPLSLPVSLGDQVSISCRSSQSIVHSNGNTYLEWYLQKPGQSPKLLIYKVSNRFSGVPDRFSGSGSGTDFTLKISRVEAEDLGVYYCFQGSHVPRTFGGGTKLEIKTERRAEVPTAHPSPSPRPAGQFQTLVVGVVGGLLGSLVLLVWVLAVICSRAARGTIGARRTGQPLKEDPSAVPVFSTEYATIVFPSGMGTSSPARRGSADGPRSAQPLRPEDGHCSWPL

SEQ ID NO: 267 - protein, 420 aa, synthetic construct
MALPVTALLLPLALLLHAARPEQKLISEEDLQVQLQQSGPELVKPGASVKMSCKASGYTFTSYHIQWVKQRPGQGLEWIGWIYPGDGSTQYNEKFKGKTTLTADKSSSTAYMLLSSLTSEDSAIYFCAREGTYYAMDYWGQGTSVTVSSGGGGSGGGGSGGGGSDVLMTQTPLSLPVSLGDQVSISCRSSQSIVHSNGNTYLEWYLQKPGQSPKLLIYKVSNRFSGVPDRFSGSGSGTDFTLKISRVEAEDLGVYYCFQGSHVPRTFGGGTKLEIKTERRAEVPTAHPSPSPRPAGQFQTLVVGVVGGLLGSLVLLVWVLAVICSRAARGTIGARRTGQPLKEDPSAVPVFSTEYATIDFQWREKTPEPPVPCVPEQTEYATIVFPSGMGTSSPARRGSADGPRSAQPLRPEDGHCSWPL

SEQ ID NO: 268 - protein, 445 aa, synthetic construct
MALPVTALLLPLALLLHAARPEQKLISEEDLQVQLQQSGPELVKPGASVKMSCKASGYTFTSYHIQWVKQRPGQGLEWIGWIYPGDGSTQYNEKFKGKTTLTADKSSSTAYMLLSSLTSEDSAIYFCAREGTYYAMDYWGQGTSVTVSSGGGGSGGGGSGGGGSDVLMTQTPLSLPVSLGDQVSISCRSSQSIVHSNGNTYLEWYLQKPGQSPKLLIYKVSNRFSGVPDRFSGSGSGTDFTLKISRVEAEDLGVYYCFQGSHVPRTFGGGTKLEIKTERRAEVPTAHPSPSPRPAGQFQTLVVGVVGGLLGSLVLLVWVLAVICSRAARGTIGARRTGQPLKEDPSAVPVFSTEYATIDFQWREKTPEPPVPCVPEQTEYATIDFQWREKTPEPPVPCVPEQTEYATIVFPSGMGTSSPARRGSADGPRSAQPLRPEDGHCSWPL

SEQ ID NO: 269 - protein, 470 aa, synthetic construct
MALPVTALLLPLALLLHAARPEQKLISEEDLQVQLQQSGPELVKPGASVKMSCKASGYTFTSYHIQWVKQRPGQGLEWIGWIYPGDGSTQYNEKFKGKTTLTADKSSSTAYMLLSSLTSEDSAIYFCAREGTYYAMDYWGQGTSVTVSSGGGGSGGGGSGGGGSDVLMTQTPLSLPVSLGDQVSISCRSSQSIVHSNGNTYLEWYLQKPGQSPKLLIYKVSNRFSGVPDRFSGSGSGTDFTLKISRVEAEDLGVYYCFQGSHVPRTFGGGTKLEIKTERRAEVPTAHPSPSPRPAGQFQTLVVGVVGGLLGSLVLLVWVLAVICSRAARGTIGARRTGQPLKEDPSAVPVFSTEYATIDFQWREKTPEPPVPCVPEQTEYATIDFQWREKTPEPPVPCVPEQTEYATIDFQWREKTPEPPVPCVPEQTEYATIVFPSGMGTSSPARRGSADGPRSAQPLRPEDGHCSWPL

SEQ ID NO: 270 - protein, 495 aa, synthetic construct
MALPVTALLLPLALLLHAARPEQKLISEEDLQVQLQQSGPELVKPGASVKMSCKASGYTFTSYHIQWVKQRPGQGLEWIGWIYPGDGSTQYNEKFKGKTTLTADKSSSTAYMLLSSLTSEDSAIYFCAREGTYYAMDYWGQGTSVTVSSGGGGSGGGGSGGGGSDVLMTQTPLSLPVSLGDQVSISCRSSQSIVHSNGNTYLEWYLQKPGQSPKLLIYKVSNRFSGVPDRFSGSGSGTDFTLKISRVEAEDLGVYYCFQGSHVPRTFGGGTKLEIKTERRAEVPTAHPSPSPRPAGQFQTLVVGVVGGLLGSLVLLVWVLAVICSRAARGTIGARRTGQPLKEDPSAVPVFSTEYATIDFQWREKTPEPPVPCVPEQTEYATIDFQWREKTPEPPVPCVPEQTEYATIDFQWREKTPEPPVPCVPEQTEYATIDFQWREKTPEPPVPCVPEQTEYATIVFPSGMGTSSPARRGSADGPRSAQPLRPEDGHCSWPL

SEQ ID NO: 271 - protein, 527 aa, synthetic construct
MALPVTALLLPLALLLHAARPEQKLISEEDLQVQLQQSGPELVKPGASVKMSCKASGYTFTSYHIQWVKQRPGQGLEWIGWIYPGDGSTQYNEKFKGKTTLTADKSSSTAYMLLSSLTSEDSAIYFCAREGTYYAMDYWGQGTSVTVSSGGGGSGGGGSGGGGSDVLMTQTPLSLPVSLGDQVSISCRSSQSIVHSNGNTYLEWYLQKPGQSPKLLIYKVSNRFSGVPDRFSGSGSGTDFTLKISRVEAEDLGVYYCFQGSHVPRTFGGGTKLEIKTERRAEVPTAHPSPSPRPAGQFQTLVVGVVGGLLGSLVLLVWVLAVICSRAARGTIGARRTGQPLKEDPSAVPVFSVDYGELDFQWREKTPEPPVPCVPEQTEYATIVFPSGMGTSSPARRGSADGPRSAQPLRPEDGHCSWPLGGGGSGGGGSCSRAARGTIGARRTGQPLKEDPSAVPVFSVDYGELDFQWREKTPEPPVPCVPEQTEYATIVFPSGMGTSSPARRGSADGPRSAQPLRPEDGHCSWPL

SEQ ID NO: 272 - protein, 470 aa, synthetic construct
MALPVTALLLPLALLLHAARPEQKLISEEDLQVQLQQSGPELVKPGASVKMSCKASGYTFTSYHIQWVKQRPGQGLEWIGWIYPGDGSTQYNEKFKGKTTLTADKSSSTAYMLLSSLTSEDSAIYFCAREGTYYAMDYWGQGTSVTVSSGGGGSGGGGSGGGGSDVLMTQTPLSLPVSLGDQVSISCRSSQSIVHSNGNTYLEWYLQKPGQSPKLLIYKVSNRFSGVPDRFSGSGSGTDFTLKISRVEAEDLGVYYCFQGSHVPRTFGGGTKLEIKTERRAEVPTAHPSPSPRPAGQFQTLVVGVVGGLLGSLVLLVWVLAVICSRAARGTIGARRTGQPLKEDPSAVPVFSVDYGELDFQWREKTPEPPVPCVPEQTEYATIDFQWREKTPEPPVPCVPEQVDYGELDFQWREKTPEPPVPCVPEQTEYATIVFPSGMGTSSPARRGSADGPRSAQPLRPEDGHCSWPL

SEQ ID NO: 273 - protein, 244 aa, synthetic construct
QVQLVESGGGVVQPGGSLRLSCAASGFTFSTYGMHWVRQAPGKGLEWVASISYDGSNKYYADSGQGRFTISRDTSKNSLYLQMNSLRAEDTAVYYCAIGIYGAYSFDYWGQGTLVTVSSGGGGSGGGGSGGGGSQSALTQPPSASGSPGQSVTISCTGTSSDVGGYKYVSWYQHHPDKAPKLMIYEVNKRPSGVPDRFSGSKSDNTASLTVSGLQAEDEADYYCSSYAGSNNWVFGGGTKLTVL

SEQ ID NO: 274 - protein, 244 aa, synthetic construct
QVQLVESGGGVVQPGGSLRLSCAASGFTFSTYGMHWVRQAPGKGLEWVASISYDGSNKYYADSGQGRFTISRDTSKNSLYLQMNSLRAEDTAVYYCAIGIYGAYSFDYWGQGTLVTVSSGGGGSGGGGSGGGGSQSALTQPPSASGSPGQSVTISCTGTSSDVGGYKYVSWYQQHPGKAPKLMIYEVNKRPSGVPDRFSGSKSGNTASLTVSGLQAEDEADYYCSSYAGSNNWVFGGGTKLTVL

SEQ ID NO: 275 - DNA, 3069 bp, synthetic construct
  source 1..3069: mol_type = other DNA
atggcgctgccagtcactgcattgttattgcctctggccctgcttctccatgcggcaaggccacaggtgcaactggttcaatctggtgctgaggtgaaaaagcccggcgcatccgtgaaagtgagctgtaaggcatcagggtacaccttcaccagctatcacatacaatgggtccgccaggcccccggacagaggttggaatggattgggtggatttacccgggtgacggctcaacccagtacaatgagaagttcaagggcagggtgactatcacacgcgatacctccgcgagcacagcttacatggagttatctagcctgagatccgaagatacggcggtgtattactgcgcgcgggaagggacctactatgccatggactattggggacaagggaccctggttaccgtgagttctgggggcgggggttccgggggagggggatctgggggtggagggagcgatgtggtaatgacccagacacctttgtctttgagtgtcacccccggacagccggcaagtatatcctgtagatcatcccaatcaatcgtgcactccaacggaaacacatacttggaatggtatctccagaaacctggacagtccccacagttgctcatctacaaagtgtcaaatcgcttttcaggcgtgcccgatcgtttcagcggctcaggctccgggacagactttacattgaagattagccgcgtagaggcagaggatgtgggcgtttactattgttttcaagggtcacacgtgccacgcacattcggcggcggtaccaaggtggaaattaagcaccccagcgacccgctggagctcgttgtgtccggaccatcagggggcccgagtagccctacaaccggccccacttctaccagtggaccggaagatcaaccacttacaccaacgggcagcgacccccagtcaggcctagggcgccacctgggtgtggtcatcgggatactggtcgctgtcatcctgcttctgctccttctcttgctcctattcctaatcctgcgccacaggagacagggcaagcactggaccagcacccagcggaaggccgactttcagcaccctgccggcgccgtgggccctgagcctaccgacaggggcctgcagtggaggagctccccagccgccgatgcccaggaggagaatctgtacgccgccgtgaagcacacccagccagaggacggcgtggagatggacacccgctccccacacgacgaggacccacaggccgtgacctacgccgaggtgaagcacagccgccccagacgcgagatggccagcccacccagccccctgtccggcgagttcctggacaccaaggacaggcaggccgaggaggaccggcagatggacaccgaggccgccgcctccgaggccccccaggacgtgacctacgcccagctgcactccctgaccctgcggagagaggccaccgagcccccacccagccaggagggcccctcccccgccgtgcctagcatctacgccaccctggccatccaccggagaaagcgtggatccggggaaggccgaggctcccttctaacatgtggagatgtcgaggaaaaccctggccctatggcgctgccagtcactgcattgttattgcctctggccctgcttctccatgcggcgcgcccagacatccagatgacccaatccccaagcagtctctcagccagcgtgggagacagggttacaatcacgtgccgcgccagccaggacgtcaacaccgctgtggcttggtatcagcaaaagcccgggaaggcaccaaagctgcttatttatagcgcctccttcttgtattctggagtgccatccaggttttccgggtcacgtagcgggactgactttaccctcaccatatccagcctccagcccgaggatttcgccacctattactgtcagcaacactacacgactccaccgacttttggacagggcactaaagtggagattaagggcagcacgagtgggagtggaaagcccggcagcggggaggggtctaccaagggagaggtccagctggttgaatccggaggcgggcttgtgcaacctggaggctccctgaggcttagttgtgccgcgtcaggattcaacattaaggatacctatattcattgggtccgacaagccccgggcaagggcttggagtgggtggccagaatctatccgaccaacggatatacaaggtacgccgattctgtgaaaggacgcttcaccatcagcgcggacacatccaaaaacacagcctatctgcagatgaactcccttcgcgccgaggatacagccgtgtactattgtagtcggtggggaggcgacggcttctacgcgatggactattggggacaaggaacactggtgactgtcagtagcactacgaccccagcacctagacctcccaccccagctccaactatagcttcccagccattgtctctccggccagaggcgtgtcgaccagccgctggaggggccgttcatacaagaggactcgatttcgcttgcgatatctacatatgggcccctcttgccgggacatgcggtgtcctgcttctaagcttggttattaccctctattgcaaaagaggacgaaagaaactgctttatatattcaagcaacctttcatgcgccccgtacagaccacgcaggaggaagatgggtgtagctgtcgcttccctgaggaagaggaaggtggatgcgagttgcgggtgaagttcagtcgatccgccgatgcgcctgcctatcagcaagggcagaaccagctttataacgagttaaaccttggccgccgggaagagtatgacgtgttggacaagcgtcgcgggagagaccctgagatgggcggaaaaccaaggagaaaaaatccacaggaaggcttatataacgagttgcagaaagacaagatggccgaggcatactccgaaatcggaatgaagggcgagcgacggcgcggcaaaggccacgatggactctatcagggcttaagcaccgccaccaaagacacctacgatgcacttcatatgcaggcactcccacctagatgataa

SEQ ID NO: 276 - protein, 1021 aa, synthetic construct
MALPVTALLLPLALLLHAARPQVQLVQSGAEVKKPGASVKVSCKASGYTFTSYHIQWVRQAPGQRLEWIGWIYPGDGSTQYNEKFKGRVTITRDTSASTAYMELSSLRSEDTAVYYCAREGTYYAMDYWGQGTLVTVSSGGGGSGGGGSGGGGSDVVMTQTPLSLSVTPGQPASISCRSSQSIVHSNGNTYLEWYLQKPGQSPQLLIYKVSNRFSGVPDRFSGSGSGTDFTLKISRVEAEDVGVYYCFQGSHVPRTFGGGTKVEIKHPSDPLELVVSGPSGGPSSPTTGPTSTSGPEDQPLTPTGSDPQSGLGRHLGVVIGILVAVILLLLLLLLLFLILRHRRQGKHWTSTQRKADFQHPAGAVGPEPTDRGLQWRSSPAADAQEENLYAAVKHTQPEDGVEMDTRSPHDEDPQAVTYAEVKHSRPRREMASPPSPLSGEFLDTKDRQAEEDRQMDTEAAASEAPQDVTYAQLHSLTLRREATEPPPSQEGPSPAVPSIYATLAIHRRKRGSGEGRGSLLTCGDVEENPGPMALPVTALLLPLALLLHAARPDIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKGSTSGSGKPGSGEGSTKGEVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 277 - DNA, 3567 bp, synthetic construct
  source 1..3567: mol_type = other DNA
atggcgctgccagtcactgcattgttattgcctctggccctgcttctccatgcggcaaggccacaggtgcaactggttcaatctggtgctgaggtgaaaaagcccggcagctctgtgaaagtgagctgtaaggcatcagggtataccttcaccagctatcacatacaatgggtccgccaggcccccggacagggattggaatggatggggtggatttacccgggtgacggctcaacccagtacaatgagaagttcaagggcaggacaactatcacagccgataagtccacgagcacagcttacatggagttatctagcctgagatccgaagatacggcggtgtattactgcgcgcgggaagggacctactatgccatggactattggggacaagggaccctggttaccgtgagttctgggggcgggggttccgggggagggggatctgggggtggagggagcgatgtggtaatgacccagacacctttgtctttgagtgtcacccccggacagccggcaagtatatcctgtagatcatcccaatcaatcgtgcactccaacggaaacacatacttggaatggtatctccagaaacctggacagtccccacagttgctcatctacaaagtgtcaaatcgcttttcaggcgtgcccgatcgtttcagcggctcaggctccgggacagactttacattgaagattagccgcgtagaggcagaggatgtgggcgtttactattgttttcaagggtcacacgtgccacgcacattcggcggcggtaccaaggtggaaattaagcaccccagcgacccgctggagctcgttgtgtccggaccatcagggggcccgagtagccctacaaccggccccacttctaccagtggaccggaagatcaaccacttacaccaacgggcagcgacccccagtcaggcctagggcgccacctgggtgtggtcatcgggatactggtcgctgtcatcctgcttctgctccttctcttgctcctattcctaatcctgcgccacaggagacagggcaagcactggaccagcacccagcggaaggccgactttcagcaccctgccggcgccgtgggccctgagcctaccgacaggggcctgcagtggaggagctccccagccgccgatgcccaggaggagaatctgtacgccgccgtgaagcacacccagccagaggacggcgtggagatggacacccgctccccacacgacgaggacccacaggccgtgacctacgccgaggtgaagcacagccgccccagacgcgagatggccagcccacccagccccctgtccggcgagttcctggacaccaaggacaggcaggccgaggaggaccggcagatggacaccgaggccgccgcctccgaggccccccaggacgtgacctacgcccagctgcactccctgaccctgcggagagaggccaccgagcccccacccagccaggagggcccctcccccgccgtgcctagcatctacgccaccctggccatccactgataacccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaaacgtctaggccccccgaaccacggggacgtggttttcctttgaaaaacacgatgataatatgatggcgctgccagtcactgcattgttattgcctctggccctgcttctccatgcggcgcgcccagacatccagatgacccaatccccaagcagtctctcagccagcgtgggagacagggttacaatcacgtgccgcgccagccaggacgtcaacaccgctgtggcttggtatcagcaaaagcccgggaaggcaccaaagctgcttatttatagcgcctccttcttgtattctggagtgccatccaggttttccgggtcacgtagcgggactgactttaccctcaccatatccagcctccagcccgaggatttcgccacctattactgtcagcaacactacacgactccaccgacttttggacagggcactaaagtggagattaagggcagcacgagtgggagtggaaagcccggcagcggggaggggtctaccaagggagaggtccagctggttgaatccggaggcgggcttgtgcaacctggaggctccctgaggcttagttgtgccgcgtcaggattcaacattaaggatacctatattcattgggtccgacaagccccgggcaagggcttggagtgggtggccagaatctatccgaccaacggatatacaaggtacgccgattctgtgaaaggacgcttcaccatcagcgcggacacatccaaaaacacagcctatctgcagatgaactcccttcgcgccgaggatacagccgtgtactattgtagtcggtggggaggcgacggcttctacgcgatggactattggggacaaggaacactggtgactgtcagtagcactacgaccccagcacctagacctcccaccccagctccaactatagcttcccagccattgtctctccggccagaggcgtgtcgaccagccgctggaggggccgttcatacaagaggactcgatttcgcttgcgatatctacatatgggcccctcttgccgggacatgcggtgtcctgcttctaagcttggttattaccctctattgcaaaagaggacgaaagaaactgctttatatattcaagcaacctttcatgcgccccgtacagaccacgcaggaggaagatgggtgtagctgtcgcttccctgaggaagaggaaggtggatgcgagttgcgggtgaagttcagtcgatccgccgatgcgcctgcctatcagcaagggcagaaccagctttataacgagttaaaccttggccgccgggaagagtatgacgtgttggacaagcgtcgcgggagagaccctgagatgggcggaaaaccaaggagaaaaaatccacaggaaggcttatataacgagttgcagaaagacaagatggccgaggcatactccgaaatcggaatgaagggcgagcgacggcgcggcaaaggccacgatggactctatcagggcttaagcaccgccaccaaagacacctacgatgcacttcatatgcaggcactcccacctagatgataa

SEQ ID NO: 278 - protein, 996 aa, synthetic construct
MALPVTALLLPLALLLHAARPQVQLVQSGAEVKKPGSSVKVSCKASGYTFTSYHIQWVRQAPGQGLEWMGWIYPGDGSTQYNEKFKGRTTITADKSTSTAYMELSSLRSEDTAVYYCAREGTYYAMDYWGQGTLVTVSSGGGGSGGGGSGGGGSDVVMTQTPLSLSVTPGQPASISCRSSQSIVHSNGNTYLEWYLQKPGQSPQLLIYKVSNRFSGVPDRFSGSGSGTDFTLKISRVEAEDVGVYYCFQGSHVPRTFGGGTKVEIKHPSDPLELVVSGPSGGPSSPTTGPTSTSGPEDQPLTPTGSDPQSGLGRHLGVVIGILVAVILLLLLLLLLFLILRHRRQGKHWTSTQRKADFQHPAGAVGPEPTDRGLQWRSSPAADAQEENLYAAVKHTQPEDGVEMDTRSPHDEDPQAVTYAEVKHSRPRREMASPPSPLSGEFLDTKDRQAEEDRQMDTEAAASEAPQDVTYAQLHSLTLRREATEPPPSQEGPSPAVPSIYATLAIHMALPVTALLLPLALLLHAARPDIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKGSTSGSGKPGSGEGSTKGEVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 279 - DNA, 3525 bp, synthetic construct
  source 1..3525: mol_type = other DNA
atggcgctgccagtcactgcattgttattgcctctggccctgcttctccatgcggcaaggccagatatagtgatgacacagtcccccgactccctggctgtctcactgggagaacgagcgacgattagttgtaagtctagccagagcgtcctgtattcaagcaataacaagaattacctcgcctggtatcagcaaaagccgggacagccacccaaactgttgatttactgggccagcacgagagagagcggagtgcccgaccgcttcagcggatccgggtcaggcacagattttaccctgactattagctcccttcaagcggaagatgtcgccgtctactattgccagcaatattacggaactccattcacattcggcggtgggaccaaagtagagataaagggtggcgggggatccggcggtggcggtagcgggggaggcgggtcccaagtgcaactagtccaatcaggtgccgaagtcaagaaaccaggtgcatccgtgaaagtgtcttgcaaagccagtggctacacttttactgactactatctgcactgggtgcgtcaagcacccggccaggggcttgaatggatgggctggattaacccttatactggagggacaaattacgctcagaagttccagggacgcgttacaatgacccgagacaccagcatcagcacagcgtacatggagttaagtgggctgacttccgacgataccgccgtgtattactgcgctcgggcaggggcctcttactatgatttttggtccggttgggtcttcgattactgggggcagggaaccctggtgacagtgtcctcaggccccacttctaccagtggaccggaagatcaaccacttacaccaacgggcagcgacccccagtcaggcctagggcgccacctgggtgtggtcatcgggatactggtcgctgtcatcctgcttctgctccttctcttgctcctattcctaatcctgcgccacaggagacagggcaagcactggaccagcacccagcggaaggccgactttcagcaccctgccggcgccgtgggccctgagcctaccgacaggggcctgcagtggaggagctccccagccgccgatgcccaggaggagaatctgtacgccgccgtgaagcacacccagccagaggacggcgtggagatggacacccgctccccacacgacgaggacccacaggccgtgacctacgccgaggtgaagcacagccgccccagacgcgagatggccagcccacccagccccctgtccggcgagttcctggacaccaaggacaggcaggccgaggaggaccggcagatggacaccgaggccgccgcctccgaggccccccaggacgtgacctacgcccagctgcactccctgaccctgcggagagaggccaccgagcccccacccagccaggagggcccctcccccgccgtgcctagcatctacgccaccctggccatccactgataacccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaaacgtctaggccccccgaaccacggggacgtggttttcctttgaaaaacacgatgataatatgatggcgctgccagtcactgcattgttattgcctctggccctgcttctccatgcggcgcgcccagacatccagatgacccaatccccaagcagtctctcagccagcgtgggagacagggttacaatcacgtgccgcgccagccaggacgtcaacaccgctgtggcttggtatcagcaaaagcccgggaaggcaccaaagctgcttatttatagcgcctccttcttgtattctggagtgccatccaggttttccgggtcacgtagcgggactgactttaccctcaccatatccagcctccagcccgaggatttcgccacctattactgtcagcaacactacacgactccaccgacttttggacagggcactaaagtggagattaagggcagcacgagtgggagtggaaagcccggcagcggggaggggtctaccaagggagaggtccagctggttgaatccggaggcgggcttgtgcaacctggaggctccctgaggcttagttgtgccgcgtcaggattcaacattaaggatacctatattcattgggtccgacaagccccgggcaagggcttggagtgggtggccagaatctatccgaccaacggatatacaaggtacgccgattctgtgaaaggacgcttcaccatcagcgcggacacatccaaaaacacagcctatctgcagatgaactcccttcgcgccgaggatacagccgtgtactattgtagtcggtggggaggcgacggcttctacgcgatggactattggggacaaggaacactggtgactgtcagtagcactacgaccccagcacctagacctcccaccccagctccaactatagcttcccagccattgtctctccggccagaggcgtgtcgaccagccgctggaggggccgttcatacaagaggactcgatttcgcttgcgatatctacatatgggcccctcttgccgggacatgcggtgtcctgcttctaagcttggttattaccctctattgcaaaagaggacgaaagaaactgctttatatattcaagcaacctttcatgcgccccgtacagaccacgcaggaggaagatgggtgtagctgtcgcttccctgaggaagaggaaggtggatgcgagttgcgggtgaagttcagtcgatccgccgatgcgcctgcctatcagcaagggcagaaccagctttataacgagttaaaccttggccgccgggaagagtatgacgtgttggacaagcgtcgcgggagagaccctgagatgggcggaaaaccaaggagaaaaaatccacaggaaggcttatataacgagttgcagaaagacaagatggccgaggcatactccgaaatcggaatgaagggcgagcgacggcgcggcaaaggccacgatggactctatcagggcttaagcaccgccaccaaagacacctacgatgcacttcatatgcaggcactcccacctagatgataa

SEQ ID NO: 280 - protein, 982 aa, synthetic construct
MALPVTALLLPLALLLHAARPDIVMTQSPDSLAVSLGERATISCKSSQSVLYSSNNKNYLAWYQQKPGQPPKLLIYWASTRESGVPDRFSGSGSGTDFTLTISSLQAEDVAVYYCQQYYGTPFTFGGGTKVEIKGGGGSGGGGSGGGGSQVQLVQSGAEVKKPGASVKVSCKASGYTFTDYYLHWVRQAPGQGLEWMGWINPYTGGTNYAQKFQGRVTMTRDTSISTAYMELSGLTSDDTAVYYCARAGASYYDFWSGWVFDYWGQGTLVTVSSGPTSTSGPEDQPLTPTGSDPQSGLGRHLGVVIGILVAVILLLLLLLLLFLILRHRRQGKHWTSTQRKADFQHPAGAVGPEPTDRGLQWRSSPAADAQEENLYAAVKHTQPEDGVEMDTRSPHDEDPQAVTYAEVKHSRPRREMASPPSPLSGEFLDTKDRQAEEDRQMDTEAAASEAPQDVTYAQLHSLTLRREATEPPPSQEGPSPAVPSIYATLAIHMALPVTALLLPLALLLHAARPDIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKGSTSGSGKPGSGEGSTKGEVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 281 - DNA, 3513 bp, synthetic construct
  source 1..3513: mol_type = other DNA
atggcgctgccagtcactgcattgttattgcctctggccctgcttctccatgcggcaaggccagatatagtgatgacacagtcccccgactccctggctgtctcactgggagaacgagcgacgattagttgtaagtctagccagagcgtcctgtattcaagcaataacaagaattacctcgcctggtatcagcaaaagccgggacagccacccaaactgttgatttactgggccagcacgagagagagcggagtgcccgaccgcttcagcggatccgggtcaggcacagattttaccctgactattagctcccttcaagcggaagatgtcgccgtctactattgccagcaatattacggaactccattcacattcggcggtgggaccaaagtagagataaagggtggcgggggatccggcggtggcggtagcgggggaggcgggtcccaagtgcaactagtccaatcaggtgccgaagtcaagaaaccaggtgcatccgtgaaagtgtcttgcaaagccagtggctacacttttactgactactatctgcactgggtgcgtcaagcacccggccaggggcttgaatggatgggctggattaacccttatactggagggacaaattacgctcagaagttccagggacgcgttacaatgacccgagacaccagcatcagcacagcgtacatggagttaagtaggctgaggtccgaagataccgccgtgtattactgcgctcgggcaggggcctcttactatgatttttggtccggttgggtcttcgattactgggggcagggaaccctggtgacagtgtcctcaaccagtggaccggaagatcaaccacttacaccaacgggcagcgacccccagtcaggcctagggcgccacctgggtgtggtcatcgggatactggtcgctgtcatcctgcttctgctccttctcttgctcctattcctaatcctgcgccacaggagacagggcaagcactggaccagcacccagcggaaggccgactttcagcaccctgccggcgccgtgggccctgagcctaccgacaggggcctgcagtggaggagctccccagccgccgatgcccaggaggagaatctgtacgccgccgtgaagcacacccagccagaggacggcgtggagatggacacccgctccccacacgacgaggacccacaggccgtgacctacgccgaggtgaagcacagccgccccagacgcgagatggccagcccacccagccccctgtccggcgagttcctggacaccaaggacaggcaggccgaggaggaccggcagatggacaccgaggccgccgcctccgaggccccccaggacgtgacctacgcccagctgcactccctgaccctgcggagagaggccaccgagcccccacccagccaggagggcccctcccccgccgtgcctagcatctacgccaccctggccatccactgataacccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaaacgtctaggccccccgaaccacggggacgtggttttcctttgaaaaacacgatgataatatgatggcgctgccagtcactgcattgttattgcctctggccctgcttctccatgcggcgcgcccagacatccagatgacccaatccccaagcagtctctcagccagcgtgggagacagggttacaatcacgtgccgcgccagccaggacgtcaacaccgctgtggcttggtatcagcaaaagcccgggaaggcaccaaagctgcttatttatagcgcctccttcttgtattctggagtgccatccaggttttccgggtcacgtagcgggactgactttaccctcaccatatccagcctccagcccgaggatttcgccacctattactgtcagcaacactacacgactccaccgacttttggacagggcactaaagtggagattaagggcagcacgagtgggagtggaaagcccggcagcggggaggggtctaccaagggagaggtccagctggttgaatccggaggcgggcttgtgcaacctggaggctccctgaggcttagttgtgccgcgtcaggattcaacattaaggatacctatattcattgggtccgacaagccccgggcaagggcttggagtgggtggccagaatctatccgaccaacggatatacaaggtacgccgattctgtgaaaggacgcttcaccatcagcgcggacacatccaaaaacacagcctatctgcagatgaactcccttcgcgccgaggatacagccgtgtactattgtagtcggtggggaggcgacggcttctacgcgatggactattggggacaaggaacactggtgactgtcagtagcactacgaccccagcacctagacctcccaccccagctccaactatagcttcccagccattgtctctccggccagaggcgtgtcgaccagccgctggaggggccgttcatacaagaggactcgatttcgcttgcgatatctacatatgggcccctcttgccgggacatgcggtgtcctgcttctaagcttggttattaccctctattgcaaaagaggacgaaagaaactgctttatatattcaagcaacctttcatgcgccccgtacagaccacgcaggaggaagatgggtgtagctgtcgcttccctgaggaagaggaaggtggatgcgagttgcgggtgaagttcagtcgatccgccgatgcgcctgcctatcagcaagggcagaaccagctttataacgagttaaaccttggccgccgggaagagtatgacgtgttggacaagcgtcgcgggagagaccctgagatgggcggaaaaccaaggagaaaaaatccacaggaaggcttatataacgagttgcagaaagacaagatggccgaggcatactccgaaatcggaatgaagggcgagcgacggcgcggcaaaggccacgatggactctatcagggcttaagcaccgccaccaaagacacctacgatgcacttcatatgcaggcactcccacctagatgataa

SEQ ID NO: 282 - protein, 978 aa, synthetic construct
MALPVTALLLPLALLLHAARPDIVMTQSPDSLAVSLGERATISCKSSQSVLYSSNNKNYLAWYQQKPGQPPKLLIYWASTRESGVPDRFSGSGSGTDFTLTISSLQAEDVAVYYCQQYYGTPFTFGGGTKVEIKGGGGSGGGGSGGGGSQVQLVQSGAEVKKPGASVKVSCKASGYTFTDYYLHWVRQAPGQGLEWMGWINPYTGGTNYAQKFQGRVTMTRDTSISTAYMELSRLRSEDTAVYYCARAGASYYDFWSGWVFDYWGQGTLVTVSSTSGPEDQPLTPTGSDPQSGLGRHLGVVIGILVAVILLLLLLLLLFLILRHRRQGKHWTSTQRKADFQHPAGAVGPEPTDRGLQWRSSPAADAQEENLYAAVKHTQPEDGVEMDTRSPHDEDPQAVTYAEVKHSRPRREMASPPSPLSGEFLDTKDRQAEEDRQMDTEAAASEAPQDVTYAQLHSLTLRREATEPPPSQEGPSPAVPSIYATLAIHMALPVTALLLPLALLLHAARPDIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKGSTSGSGKPGSGEGSTKGEVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 283 - protein, 113 aa, synthetic construct; identical to SEQ ID NO: 165

SEQ ID NO: 284 - protein, 125 aa, synthetic construct
QVQLVQSGAEVKKPGASVKVSCKASGYTFTDYYLHWVRQAPGQGLEWMGWINPYTGGTNYAQKFQGRVTMTRDTSISTAYMELSRLRSEDTAVYYCARAGASYYDFWSGWVFDYWGQGTLVTVSS

SEQ ID NO: 285 - protein, 253 aa, synthetic construct
DIVMTQSPDSLAVSLGERATISCKSSQSVLYSSNNKNYLAWYQQKPGQPPKLLIYWASTRESGVPDRFSGSGSGTDFTLTISSLQAEDVAVYYCQQYYGTPFTFGGGTKVEIKGGGGSGGGGSGGGGSQVQLVQSGAEVKKPGASVKVSCKASGYTFTDYYLHWVRQAPGQGLEWMGWINPYTGGTNYAQKFQGRVTMTRDTSISTAYMELSGLTSDDTAVYYCARAGASYYDFWSGWVFDYWGQGTLVTVSS

SEQ ID NO: 286 - protein, 253 aa, synthetic construct
DIVMTQSPDSLAVSLGERATISCKSSQSVLYSSNNKNYLAWYQQKPGQPPKLLIYWASTRESGVPDRFSGSGSGTDFTLTISSLQAEDVAVYYCQQYYGTPFTFGGGTKVEIKGGGGSGGGGSGGGGSQVQLVQSGAEVKKPGASVKVSCKASGYTFTDYYLHWVRQAPGQGLEWMGWINPYTGGTNYAQKFQGRVTMTRDTSISTAYMELSRLRSEDTAVYYCARAGASYYDFWSGWVFDYWGQGTLVTVSS

SEQ ID NO: 287 - protein, 245 aa, synthetic construct
QVQLVQSGAEVKKPGSSVKVSCKASGYTFTSYHIQWVRQAPGQGLEWMGWIYPGDGSTQYNEKFKGRTTITADKSTSTAYMELSSLRSEDTAVYYCAREGTYYAMDYWGQGTLVTVSSGGGGSGGGGSGGGGSDVVMTQTPLSLSVTPGQPASISCRSSQSIVHSNGNTYLEWYLQKPGQSPQLLIYKVSNRFSGVPDRFSGSGSGTDFTLKISRVEAEDVGVYYCFQGSHVPRTFGGGTKVEIK

SEQ ID NO: 288 - protein, 245 aa, synthetic construct
QVQLVQSGAEVKKPGASVKVSCKASGYTFTSYHIQWVRQAPGQRLEWIGWIYPGDGSTQYNEKFKGRVTITRDTSASTAYMELSSLRSEDTAVYYCAREGTYYAMDYWGQGTLVTVSSGGGGSGGGGSGGGGSDVVMTQTPLSLSVTPGQPASISCRSSQSIVHSNGNTYLEWYLQKPGQSPQLLIYKVSNRFSGVPDRFSGSGSGTDFTLKISRVEAEDVGVYYCFQGSHVPRTFGGGTKVEIK

SEQ ID NO: 289 - protein, 26 aa, synthetic construct
TSGPEDQPLTPTGSDPQSGLGRHLGV

SEQ ID NO: 290 - protein, 47 aa, synthetic construct
GAISLAPKAQIKESLRAELRVTERRAEVPTAHPSPSPRPAGQFQTLV

SEQ ID NO: 291 - protein, 42 aa, synthetic construct
APKAQIKESLRAELRVTERRAEVPTAHPSPSPRPAGQFQTLV

SEQ ID NO: 292 - protein, 36 aa, synthetic construct
KESLRAELRVTERRAEVPTAHPSPSPRPAGQFQTLV

SEQ ID NO: 293 - protein, 30 aa, synthetic construct
ELRVTERRAEVPTAHPSPSPRPAGQFQTLV

SEQ ID NO: 294 - protein, 26 aa, synthetic construct
TERRAEVPTAHPSPSPRPAGQFQTLV

SEQ ID NO: 295 - protein, 20 aa, synthetic construct
VPTAHPSPSPRPAGQFQTLV

SEQ ID NO: 296 - protein, 168 aa, synthetic construct
LRHRRQGKHWTSTQRKADFQHPAGAVGPEPTDRGLQWRSSPAADAQEENLYAAVKHTQPEDGVEMDTRSPHDEDPQANLYAAVKHSRPRREMASPPSPLSGEFLDTKDRQAEEDRQMDTEAAASEAPQDNLYAAVHSLTLRREATEPPPSQEGPSPAVPNLYAAVAIH

SEQ ID NO: 297 - protein, 168 aa, synthetic construct
LRHRRQGKHWTSTQRKADFQHPAGAVGPEPTDRGLQWRSSPAADAQEEVTYAEVKHTQPEDGVEMDTRSPHDEDPQAVTYAEVKHSRPRREMASPPSPLSGEFLDTKDRQAEEDRQMDTEAAASEAPQDVTYAEVHSLTLRREATEPPPSQEGPSPAVPVTYAEVAIH

SEQ ID NO: 298 - protein, 168 aa, synthetic construct
LRHRRQGKHWTSTQRKADFQHPAGAVGPEPTDRGLQWRSSPAADAQEEVTYAQLKHTQPEDGVEMDTRSPHDEDPQAVTYAQLKHSRPRREMASPPSPLSGEFLDTKDRQAEEDRQMDTEAAASEAPQDVTYAQLHSLTLRREATEPPPSQEGPSPAVPVTYAQLAIH

SEQ ID NO: 299 - protein, 168 aa, synthetic construct
LRHRRQGKHWTSTQRKADFQHPAGAVGPEPTDRGLQWRSSPAADAQEESIYATLKHTQPEDGVEMDTRSPHDEDPQASIYATLKHSRPRREMASPPSPLSGEFLDTKDRQAEEDRQMDTEAAASEAPQDSIYATLHSLTLRREATEPPPSQEGPSPAVPSIYATLAIH

SEQ ID NO: 300 - protein, 168 aa, synthetic construct
LRHRRQGKHWTSTQRKADFQHPAGAVGPEPTDRGLQWRSSPAADAQEEVTYAQLKHTQPEDGVEMDTRSPHDEDPQASIYATLKHSRPRREMASPPSPLSGEFLDTKDRQAEEDRQMDTEAAASEAPQDVTYAQLHSLTLRREATEPPPSQEGPSPAVPSIYATLAIH

SEQ ID NO: 301 - protein, 168 aa, synthetic construct
LRHRRQGKHWTSTQRKADFQHPAGAVGPEPTDRGLQWRSSPAADAQEETEYATIKHTQPEDGVEMDTRSPHDEDPQATEYATIKHSRPRREMASPPSPLSGEFLDTKDRQAEEDRQMDTEAAASEAPQDVTYAQLHSLTLRREATEPPPSQEGPSPAVPSIYATLAIH

SEQ ID NO: 302 - protein, 168 aa, synthetic construct
LRHRRQGKHWTSTQRKADFQHPAGAVGPEPTDRGLQWRSSPAADAQEEVTYAQLKHTQPEDGVEMDTRSPHDEDPQASIYATLKHSRPRREMASPPSPLSGEFLDTKDRQAEEDRQMDTEAAASEAPQDTEYATIHSLTLRREATEPPPSQEGPSPAVPTEYATIAIH

SEQ ID NO: 303 - skipped

SEQ ID NO: 304 - protein, 168 aa, synthetic construct
LRHRRQGKHWTSTQRKADFQHPAGAVGPEPTDRGLQWRSSPAADAQEEVTYAQLKHTQPEDGVEMDTRSPHDEDPQATEYATIKHSRPRREMASPPSPLSGEFLDTKDRQAEEDRQMDTEAAASEAPQDTEYATIHSLTLRREATEPPPSQEGPSPAVPSIYATLAIH

SEQ ID NO: 305 - protein, 489 aa, synthetic construct
MALPVTALLLPLALLLHAARPDIVMTQSPDSLAVSLGERATISCKSSQSVLYSSNNKNYLAWYQQKPGQPPKLLIYWASTRESGVPDRFSGSGSGTDFTLTISSLQAEDVAVYYCQQYYGTPFTFGGGTKVEIKGGGGSGGGGSGGGGSQVQLVQSGAEVKKPGASVKVSCKASGYTFTDYYLHWVRQAPGQGLEWMGWINPYTGGTNYAQKFQGRVTMTRDTSISTAYMELSRLRSEDTAVYYCARAGASYYDFWSGWVFDYWGQGTLVTVSSTSGPEDQPLTPTGSDPQSGLGRHLGVVIGILVAVILLLLLLLLLFLILRHRRQGKHWTSTQRKADFQHPAGAVGPEPTDRGLQWRSSPAADAQEENLYAAVKHTQPEDGVEMDTRSPHDEDPQAVTYAEVKHSRPRREMASPPSPLSGEFLDTKDRQAEEDRQMDTEAAASEAPQDVTYAQLHSLTLRREATEPPPSQEGPSPAVPSIYATLAIH

SEQ ID NO: 306 - protein, 20 aa, synthetic construct
MSVPTQVLGLLLLWLTDARC

SEQ ID NO: 307 - protein, 107 aa, synthetic construct [sequence omitted]

SEQ ID NO: 308 - protein, 120 aa, synthetic construct
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGGYAMDYWGQGTLVTVSS

SEQ ID NO: 309 - protein, 123 aa, synthetic construct
QVQLVQSGGGLVQAGGSLRLSCAASGRTFSSYAMAWFRQAPGKEREFVAAISWSGANIYVADSVKGRFTISRDNAKDTVYLQMNSLKPEDTAVYYCAVKLGFAPVEERQYDYWGQGTQVTVSS

SEQ ID NO: 310 - protein, 112 aa, synthetic construct [sequence omitted]

SEQ ID NO: 311 - protein, 123 aa, synthetic construct
QVQLQQSGAEVKKPGSSVKVSCKASGYTFTDYYIYWVRQAPGQGLEWIGGINPVTQRPVFNEKFKTRVTITVDESTNTAYMELSSLRSEDTAFYFCARQGLWFDSDGRGFDFWGQGSTVTVSS

SEQ ID NO: 312 - protein, 124 aa, synthetic construct
EVQQASGGGLVQAGGSLRLSCAASGRTETTSAIAWFRQAPGKEREFVAQISASGLGINYSGTVKGRFTISRDADKTTVYLQMNSLTPEDTAVYYCAAGFHYIAAIRRTTDFHFWGPGTLVTVSS

SEQ ID NO: 313 - protein, 107 aa, synthetic construct
EIVLTQSPATLSLSPGERATISCRASQSVSSNFAWYQQRPGQAPRLLIYDASNRATGIPPRFSGSGSGTDFTLTISSLEPEDFAAYYCHQRSNWLYTFGQGTKVDIK

SEQ ID NO: 314 - protein, 117 aa, synthetic construct
QVQLQQSGAEVKKPGASVKVSCKASGYTFTGYYMHWVRQAPGQGLEWMGRINPNSGGTNYAQKFQGRVTMTRDTSISTAYMELSRLRSEDTAVYYCARGRYYGMDVWGQGTMVTVSS

SEQ ID NO: 315 - protein, 106 aa, synthetic construct
DIVMTQSPSSLSASVGDRVTITCRASQSIRYYLSWYQQKPGKAPKLLIYTASILQNGVPSRFSGSGSGTDFTLTISSLQPEDFATYYCLQTYTTPDFGPGTKVEIK

SEQ ID NO: 316 - protein, 115 aa, synthetic construct
QVQLVQSGAEVEKPGASVKVSCKASGYTFTDYYMHWVRQAPGQGLEWMGWINPNSGGTNYAQKFQGRVTMTRDTSISTAYMELSRLRSDDTAVYYCASGWDFDYWGQGTLVTVSS

SEQ ID NO: 317 - protein, 112 aa, synthetic construct
DIQMTQSPSSLSASVGDRVTITCKSSQSVLYSSNQKNYLAWFQQKPGKAPKLLIYWASTRESGVPSRFSGSGSGTDFTLTISSLQPEDFATYFCHQYLSSYTFGQGTKVEIK

SEQ ID NO: 318 - protein, 116 aa, synthetic construct
EVQLVESGGGLVQPGGSLRLSCAASGYTFTTYWMHWVRQAPGKGLEWVGYIRPSTGYTEYNQKFKDRFTISADTSKNTAYLQMNSLRAEDTAVYYCARSRWLLDYWGQGTLVTVSS

SEQ ID NO: 319 - protein, 27 aa, synthetic construct
FWVLVVVGGVLACYSLLVTVAFIIFWV

SEQ ID NO: 320 - protein, 24 aa, synthetic construct
IYIWAPLAGTCGVLLLSLVITLYC

SEQ ID NO: 321 - DNA, 2991 bp, synthetic construct
  source 1..2991: mol_type = other DNA
atggcgctgccagtcactgcattgttattgcctctggccctgcttctccatgcggcaaggccacaggtgcaactggttcaatctggtgctgaggtgaaaaagcccggcgcatccgtgaaagtgagctgtaaggcatcagggtacaccttcaccagctatcacatacaatgggtccgccaggcccccggacagaggttggaatggattgggtggatttacccgggtgacggctcaacccagtacaatgagaagttcaagggcagggtgactatcacacgcgatacctccgcgagcacagcttacatggagttatctagcctgagatccgaagatacggcggtgtattactgcgcgcgggaagggacctactatgccatggactattggggacaagggaccctggttaccgtgagttctgggggcgggggttccgggggagggggatctgggggtggagggagcgatgtggtaatgacccagacacctttgtctttgagtgtcacccccggacagccggcaagtatatcctgtagatcatcccaatcaatcgtgcactccaacggaaacacatacttggaatggtatctccagaaacctggacagtccccacagttgctcatctacaaagtgtcaaatcgcttttcaggcgtgcccgatcgtttcagcggctcaggctccgggacagactttacattgaagattagccgcgtagaggcagaggatgtgggcgtttactattgttttcaagggtcacacgtgccacgcacattcggcggcggtaccaaggtggaaattaagggccccacttctaccagtggaccggaagatcaaccacttacaccaacgggcagcgacccccagtcaggcctagggcgccacctgggtgtggtcatcgggatactggtcgctgtcatcctgcttctgctccttctcttgctcctattcctaatcctgcgccacaggagacagggcaagcactggaccagcacccagcggaaggccgactttcagcaccctgccggcgccgtgggccctgagcctaccgacaggggcctgcagtggaggagctccccagccgccgatgcccaggaggagaatctgtacgccgccgtgaagcacacccagccagaggacggcgtggagatggacacccgctccccacacgacgaggacccacaggccgtgacctacgccgaggtgaagcacagccgccccagacgcgagatggccagcccacccagccccctgtccggcgagttcctggacaccaaggacaggcaggccgaggaggaccggcagatggacaccgaggccgccgcctccgaggccccccaggacgtgacctacgcccagctgcactccctgaccctgcggagagaggccaccgagcccccacccagccaggagggcccctcccccgccgtgcctagcatctacgccaccctggccatccacggatccggggaaggccgaggctcccttctaacatgtggagatgtcgaggaaaaccctggccctatggcgctgccagtcactgcattgttattgcctctggccctgcttctccatgcggcgcgcccagacatccagatgacccaatccccaagcagtctctcagccagcgtgggagacagggttacaatcacgtgccgcgccagccaggacgtcaacaccgctgtggcttggtatcagcaaaagcccgggaaggcaccaaagctgcttatttatagcgcctccttcttgtattctggagtgccatccaggttttccgggtcacgtagcgggactgactttaccctcaccatatccagcctccagcccgaggatttcgccacctattactgtcagcaacactacacgactccaccgacttttggacagggcactaaagtggagattaagggcagcacgagtgggagtggaaagcccggcagcggggaggggtctaccaagggagaggtccagctggttgaatccggaggcgggcttgtgcaacctggaggctccctgaggcttagttgtgccgcgtcaggattcaacattaaggatacctatattcattgggtccgacaagccccgggcaagggcttggagtgggtggccagaatctatccgaccaacggatatacaaggtacgccgattctgtgaaaggacgcttcaccatcagcgcggacacatccaaaaacacagcctatctgcagatgaactcccttcgcgccgaggatacagccgtgtactattgtagtcggtggggaggcgacggcttctacgcgatggactattggggacaaggaacactggtgactgtcagtagcactacgaccccagcacctagacctcccaccccagctccaactatagcttcccagccattgtctctccggccagaggcgtgtcgaccagccgctggaggggccgttcatacaagaggactcgatttcgcttgcgatatctacatatgggcccctcttgccgggacatgcggtgtcctgcttctaagcttggttattaccctctattgcaaaagaggacgaaagaaactgctttatatattcaagcaacctttcatgcgccccgtacagaccacgcaggaggaagatgggtgtagctgtcgcttccctgaggaagaggaaggtggatgcgagttgcgggtgaagttcagtcgatccgccgatgcgcctgcctatcagcaagggcagaaccagctttataacgagttaaaccttggccgccgggaagagtatgacgtgttggacaagcgtcgcgggagagaccctgagatgggcggaaaaccaaggagaaaaaatccacaggaaggcttatataacgagttgcagaaagacaagatggccgaggcatactccgaaatcggaatgaagggcgagcgacggcgcggcaaaggccacgatggactctatcagggcttaagcaccgccaccaaagacacctacgatgcacttcatatgcaggcactcccacctagatgataa

SEQ ID NO: 322 - protein, 995 aa, synthetic construct
MALPVTALLLPLALLLHAARPQVQLVQSGAEVKKPGASVKVSCKASGYTFTSYHIQWVRQAPGQRLEWIGWIYPGDGSTQYNEKFKGRVTITRDTSASTAYMELSSLRSEDTAVYYCAREGTYYAMDYWGQGTLVTVSSGGGGSGGGGSGGGGSDVVMTQTPLSLSVTPGQPASISCRSSQSIVHSNGNTYLEWYLQKPGQSPQLLIYKVSNRFSGVPDRFSGSGSGTDFTLKISRVEAEDVGVYYCFQGSHVPRTFGGGTKVEIKGPTSTSGPEDQPLTPTGSDPQSGLGRHLGVVIGILVAVILLLLLLLLLFLILRHRRQGKHWTSTQRKADFQHPAGAVGPEPTDRGLQWRSSPAADAQEENLYAAVKHTQPEDGVEMDTRSPHDEDPQAVTYAEVKHSRPRREMASPPSPLSGEFLDTKDRQAEEDRQMDTEAAASEAPQDVTYAQLHSLTLRREATEPPPSQEGPSPAVPSIYATLAIHGSGEGRGSLLTCGDVEENPGPMALPVTALLLPLALLLHAARPDIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKGSTSGSGKPGSGEGSTKGEVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 323 - DNA, 3015 bp, synthetic construct
  source 1..3015: mol_type = other DNA
atggcgctgccagtcactgcattgttattgcctctggccctgcttctccatgcggcaaggccagatatagtgatgacacagtcccccgactccctggctgtctcactgggagaacgagcgacgattagttgtaagtctagccagagcgtcctgtattcaagcaataacaagaattacctcgcctggtatcagcaaaagccgggacagccacccaaactgttgatttactgggccagcacgagagagagcggagtgcccgaccgcttcagcggatccgggtcaggcacagattttaccctgactattagctcccttcaagcggaagatgtcgccgtctactattgccagcaatattacggaactccattcacattcggcggtgggaccaaagtagagataaagggtggcgggggatccggcggtggcggtagcgggggaggcgggtcccaagtgcaactagtccaatcaggtgccgaagtcaagaaaccaggtgcatccgtgaaagtgtcttgcaaagccagtggctacacttttactgactactatctgcactgggtgcgtcaagcacccggccaggggcttgaatggatgggctggattaacccttatactggagggacaaattacgctcagaagttccagggacgcgttacaatgacccgagacaccagcatcagcacagcgtacatggagttaagtgggctgacttccgacgataccgccgtgtattactgcgctcgggcaggggcctcttactatgatttttggtccggttgggtcttcgattactgggggcagggaaccctggtgacagtgtcctcaggccccacttctaccagtggaccggaagatcaaccacttacaccaacgggcagcgacccccagtcaggcctagggcgccacctgggtgtggtcatcgggatactggtcgctgtcatcctgcttctgctccttctcttgctcctattcctaatcctgcgccacaggagacagggcaagcactggaccagcacccagcggaaggccgactttcagcaccctgccggcgccgtgggccctgagcctaccgacaggggcctgcagtggaggagctccccagccgccgatgcccaggaggagaatctgtacgccgccgtgaagcacacccagccagaggacggcgtggagatggacacccgctccccacacgacgaggacccacaggccgtgacctacgccgaggtgaagcacagccgccccagacgcgagatggccagcccacccagccccctgtccggcgagttcctggacaccaaggacaggcaggccgaggaggaccggcagatggacaccgaggccgccgcctccgaggccccccaggacgtgacctacgcccagctgcactccctgaccctgcggagagaggccaccgagcccccacccagccaggagggcccctcccccgccgtgcctagcatctacgccaccctggccatccacggatccggggaaggccgaggctcccttctaacatgtggagatgtcgaggaaaaccctggccctatggcgctgccagtcactgcattgttattgcctctggccctgcttctccatgcggcgcgcccagacatccagatgacccaatccccaagcagtctctcagccagcgtgggagacagggttacaatcacgtgccgcgccagccaggacgtcaacaccgctgtggcttggtatcagcaaaagcccgggaaggcaccaaagctgcttatttatagcgcctccttcttgtattctggagtgccatccaggttttccgggtcacgtagcgggactgactttaccctcaccatatccagcctccagcccgaggatttcgccacctattactgtcagcaacactacacgactccaccgacttttggacagggcactaaagtggagattaagggcagcacgagtgggagtggaaagcccggcagcggggaggggtctaccaagggagaggtccagctggttgaatccggaggcgggcttgtgcaacctggaggctccctgaggcttagttgtgccgcgtcaggattcaacattaaggatacctatattcattgggtccgacaagccccgggcaagggcttggagtgggtggccagaatctatccgaccaacggatatacaaggtacgccgattctgtgaaaggacgcttcaccatcagcgcggacacatccaaaaacacagcctatctgcagatgaactcccttcgcgccgaggatacagccgtgtactattgtagtcggtggggaggcgacggcttctacgcgatggactattggggacaaggaacactggtgactgtcagtagcactacgaccccagcacctagacctcccaccccagctccaactatagcttcccagccattgtctctccggccagaggcgtgtcgaccagccgctggaggggccgttcatacaagaggactcgatttcgcttgcgatatctacatatgggcccctcttgccgggacatgcggtgtcctgcttctaagcttggttattaccctctattgcaaaagaggacgaaagaaactgctttatatattcaagcaacctttcatgcgccccgtacagaccacgcaggaggaagatgggtgtagctgtcgcttccctgaggaagaggaaggtggatgcgagttgcgggtgaagttcagtcgatccgccgatgcgcctgcctatcagcaagggcagaaccagctttataacgagttaaaccttggccgccgggaagagtatgacgtgttggacaagcgtcgcgggagagaccctgagatgggcggaaaaccaaggagaaaaaatccacaggaaggcttatataacgagttgcagaaagacaagatggccgaggcatactccgaaatcggaatgaagggcgagcgacggcgcggcaaaggccacgatggactctatcagggcttaagcaccgccaccaaagacacctacgatgcacttcatatgcaggcactcccacctagatgataa

SEQ ID NO: 324 - protein, 1003 aa, synthetic construct
MALPVTALLLPLALLLHAARPDIVMTQSPDSLAVSLGERATISCKSSQSVLYSSNNKNYLAWYQQKPGQPPKLLIYWASTRESGVPDRFSGSGSGTDFTLTISSLQAEDVAVYYCQQYYGTPFTFGGGTKVEIKGGGGSGGGGSGGGGSQVQLVQSGAEVKKPGASVKVSCKASGYTFTDYYLHWVRQAPGQGLEWMGWINPYTGGTNYAQKFQGRVTMTRDTSISTAYMELSGLTSDDTAVYYCARAGASYYDFWSGWVFDYWGQGTLVTVSSGPTSTSGPEDQPLTPTGSDPQSGLGRHLGVVIGILVAVILLLLLLLLLFLILRHRRQGKHWTSTQRKADFQHPAGAVGPEPTDRGLQWRSSPAADAQEENLYAAVKHTQPEDGVEMDTRSPHDEDPQAVTYAEVKHSRPRREMASPPSPLSGEFLDTKDRQAEEDRQMDTEAAASEAPQDVTYAQLHSLTLRREATEPPPSQEGPSPAVPSIYATLAIHGSGEGRGSLLTCGDVEENPGPMALPVTALLLPLALLLHAARPDIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKGSTSGSGKPGSGEGSTKGEVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 325 - DNA, 3003 bp, synthetic construct
  source 1..3003: mol_type = other DNA
atggcgctgccagtcactgcattgttattgcctctggccctgcttctccatgcggcaaggccagatatagtgatgacacagtcccccgactccctggctgtctcactgggagaacgagcgacgattagttgtaagtctagccagagcgtcctgtattcaagcaataacaagaattacctcgcctggtatcagcaaaagccgggacagccacccaaactgttgatttactgggccagcacgagagagagcggagtgcccgaccgcttcagcggatccgggtcaggcacagattttaccctgactattagctcccttcaagcggaagatgtcgccgtctactattgccagcaatattacggaactccattcacattcggcggtgggaccaaagtagagataaagggtggcgggggatccggcggtggcggtagcgggggaggcgggtcccaagtgcaactagtccaatcaggtgccgaagtcaagaaaccaggtgcatccgtgaaagtgtcttgcaaagccagtggctacacttttactgactactatctgcactgggtgcgtcaagcacccggccaggggcttgaatggatgggctggattaacccttatactggagggacaaattacgctcagaagttccagggacgcgttacaatgacccgagacaccagcatcagcacagcgtacatggagttaagtaggctgaggtccgaagataccgccgtgtattactgcgctcgggcaggggcctcttactatgatttttggtccggttgggtcttcgattactgggggcagggaaccctggtgacagtgtcctcaaccagtggaccggaagatcaaccacttacaccaacgggcagcgacccccagtcaggcctagggcgccacctgggtgtggtcatcgggatactggtcgctgtcatcctgcttctgctccttctcttgctcctattcctaatcctgcgccacaggagacagggcaagcactggaccagcacccagcggaaggccgactttcagcaccctgccggcgccgtgggccctgagcctaccgacaggggcctgcagtggaggagctccccagccgccgatgcccaggaggagaatctgtacgccgccgtgaagcacacccagccagaggacggcgtggagatggacacccgctccccacacgacgaggacccacaggccgtgacctacgccgaggtgaagcacagccgccccagacgcgagatggccagcccacccagccccctgtccggcgagttcctggacaccaaggacaggcaggccgaggaggaccggcagatggacaccgaggccgccgcctccgaggccccccaggacgtgacctacgcccagctgcactccctgaccctgcggagagaggccaccgagcccccacccagccaggagggcccctcccccgccgtgcctagcatctacgccaccctggccatccacggatccggggaaggccgaggctcccttctaacatgtggagatgtcgaggaaaaccctggccctatggcgctgccagtcactgcattgttattgcctctggccctgcttctccatgcggcgcgcccagacatccagatgacccaatccccaagcagtctctcagccagcgtgggagacagggttacaatcacgtgccgcgccagccaggacgtcaacaccgctgtggcttggtatcagcaaaagcccgggaaggcaccaaagctgcttatttatagcgcctccttcttgtattctggagtgccatccaggttttccgggtcacgtagcgggactgactttaccctcaccatatccagcctccagcccgaggatttcgccacctattactgtcagcaacactacacgactccaccgacttttggacagggcactaaagtggagattaagggcagcacgagtgggagtggaaagcccggcagcggggaggggtctaccaagggagaggtccagctggttgaatccggaggcgggcttgtgcaacctggaggctccctgaggcttagttgtgccgcgtcaggattcaacattaaggatacctatattcattgggtccgacaagccccgggcaagggcttggagtgggtggccagaatctatccgaccaacggatatacaaggtacgccgattctgtgaaaggacgcttcaccatcagcgcggacacatccaaaaacacagcctatctgcagatgaactcccttcgcgccgaggatacagccgtgtactattgtagtcggtggggaggcgacggcttctacgcgatggactattggggacaaggaacactggtgactgtcagtagcactacgaccccagcacctagacctcccaccccagctccaactatagcttcccagccattgtctctccggccagaggcgtgtcgaccagccgctggaggggccgttcatacaagaggactcgatttcgcttgcgatatctacatatgggcccctcttgccgggacatgcggtgtcctgcttctaagcttggttattaccctctattgcaaaagaggacgaaagaaactgctttatatattcaagcaacctttcatgcgccccgtacagaccacgcaggaggaagatgggtgtagctgtcgcttccctgaggaagaggaaggtggatgcgagttgcgggtgaagttcagtcgatccgccgatgcgcctgcctatcagcaagggcagaaccagctttataacgagttaaaccttggccgccgggaagagtatgacgtgttggacaagcgtcgcgggagagaccctgagatgggcggaaaaccaaggagaaaaaatccacaggaaggcttatataacgagttgcagaaagacaagatggccgaggcatactccgaaatcggaatgaagggcgagcgacggcgcggcaaaggccacgatggactctatcagggcttaagcaccgccaccaaagacacctacgatgcacttcatatgcaggcactcccacctagatgataa

SEQ ID NO: 326 - protein, 999 aa, synthetic construct
MALPVTALLLPLALLLHAARPDIVMTQSPDSLAVSLGERATISCKSSQSVLYSSNNKNYLAWYQQKPGQPPKLLIYWASTRESGVPDRFSGSGSGTDFTLTISSLQAEDVAVYYCQQYYGTPFTFGGGTKVEIKGGGGSGGGGSGGGGSQVQLVQSGAEVKKPGASVKVSCKASGYTFTDYYLHWVRQAPGQGLEWMGWINPYTGGTNYAQKFQGRVTMTRDTSISTAYMELSRLRSEDTAVYYCARAGASYYDFWSGWVFDYWGQGTLVTVSSTSGPEDQPLTPTGSDPQSGLGRHLGVVIGILVAVILLLLLLLLLFLILRHRRQGKHWTSTQRKADFQHPAGAVGPEPTDRGLQWRSSPAADAQEENLYAAVKHTQPEDGVEMDTRSPHDEDPQAVTYAEVKHSRPRREMASPPSPLSGEFLDTKDRQAEEDRQMDTEAAASEAPQDVTYAQLHSLTLRREATEPPPSQEGPSPAVPSIYATLAIHGSGEGRGSLLTCGDVEENPGPMALPVTALLLPLALLLHAARPDIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKGSTSGSGKPGSGEGSTKGEVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 327 - protein, 481 aa, synthetic construct
MALPVTALLLPLALLLHAARPQVQLVQSGAEVKKPGASVKVSCKASGYTFTSYHIQWVRQAPGQRLEWIGWIYPGDGSTQYNEKFKGRVTITRDTSASTAYMELSSLRSEDTAVYYCAREGTYYAMDYWGQGTLVTVSSGGGGSGGGGSGGGGSDVVMTQTPLSLSVTPGQPASISCRSSQSIVHSNGNTYLEWYLQKPGQSPQLLIYKVSNRFSGVPDRFSGSGSGTDFTLKISRVEAEDVGVYYCFQGSHVPRTFGGGTKVEIKTERRAEVPTAHPSPSPRPAGQFQTLVVGVVGGLLGSLVLLVWVLAVILRHRRQGKHWTSTQRKADFQHPAGAVGPEPTDRGLQWRSSPAADAQEENLYAAVKHTQPEDGVEMDTRSPHDEDPQANLYAAVKHSRPRREMASPPSPLSGEFLDTKDRQAEEDRQMDTEAAASEAPQDNLYAAVHSLTLRREATEPPPSQEGPSPAVPNLYAAVAIH

SEQ ID NO: 328 - protein, 481 aa, synthetic construct
MALPVTALLLPLALLLHAARPQVQLVQSGAEVKKPGASVKVSCKASGYTFTSYHIQWVRQAPGQRLEWIGWIYPGDGSTQYNEKFKGRVTITRDTSASTAYMELSSLRSEDTAVYYCAREGTYYAMDYWGQGTLVTVSSGGGGSGGGGSGGGGSDVVMTQTPLSLSVTPGQPASISCRSSQSIVHSNGNTYLEWYLQKPGQSPQLLIYKVSNRFSGVPDRFSGSGSGTDFTLKISRVEAEDVGVYYCFQGSHVPRTFGGGTKVEIKTERRAEVPTAHPSPSPRPAGQFQTLVVGVVGGLLGSLVLLVWVLAVILRHRRQGKHWTSTQRKADFQHPAGAVGPEPTDRGLQWRSSPAADAQEEVTYAEVKHTQPEDGVEMDTRSPHDEDPQAVTYAEVKHSRPRREMASPPSPLSGEFLDTKDRQAEEDRQMDTEAAASEAPQDVTYAEVHSLTLRREATEPPPSQEGPSPAVPVTYAEVAIH

SEQ ID NO: 329 - protein, 481 aa, synthetic construct
MALPVTALLLPLALLLHAARPQVQLVQSGAEVKKPGASVKVSCKASGYTFTSYHIQWVRQAPGQRLEWIGWIYPGDGSTQYNEKFKGRVTITRDTSASTAYMELSSLRSEDTAVYYCAREGTYYAMDYWGQGTLVTVSSGGGGSGGGGSGGGGSDVVMTQTPLSLSVTPGQPASISCRSSQSIVHSNGNTYLEWYLQKPGQSPQLLIYKVSNRFSGVPDRFSGSGSGTDFTLKISRVEAEDVGVYYCFQGSHVPRTFGGGTKVEIKTERRAEVPTAHPSPSPRPAGQFQTLVVGVVGGLLGSLVLLVWVLAVILRHRRQGKHWTSTQRKADFQHPAGAVGPEPTDRGLQWRSSPAADAQEEVTYAQLKHTQPEDGVEMDTRSPHDEDPQAVTYAQLKHSRPRREMASPPSPLSGEFLDTKDRQAEEDRQMDTEAAASEAPQDVTYAQLHSLTLRREATEPPPSQEGPSPAVPVTYAQLAIH

SEQ ID NO: 330 - protein, 481 aa, synthetic construct
MALPVTALLLPLALLLHAARPQVQLVQSGAEVKKPGASVKVSCKASGYTFTSYHIQWVRQAPGQRLEWIGWIYPGDGSTQYNEKFKGRVTITRDTSASTAYMELSSLRSEDTAVYYCAREGTYYAMDYWGQGTLVTVSSGGGGSGGGGSGGGGSDVVMTQTPLSLSVTPGQPASISCRSSQSIVHSNGNTYLEWYLQKPGQSPQLLIYKVSNRFSGVPDRFSGSGSGTDFTLKISRVEAEDVGVYYCFQGSHVPRTFGGGTKVEIKTERRAEVPTAHPSPSPRPAGQFQTLVVGVVGGLLGSLVLLVWVLAVILRHRRQGKHWTSTQRKADFQHPAGAVGPEPTDRGLQWRSSPAADAQEESIYATLKHTQPEDGVEMDTRSPHDEDPQASIYATLKHSRPRREMASPPSPLSGEFLDTKDRQAEEDRQMDTEAAASEAPQDSIYATLHSLTLRREATEPPPSQEGPSPAVPSIYATLAIH

SEQ ID NO: 331 - protein, 481 aa, synthetic construct
MALPVTALLLPLALLLHAARPQVQLVQSGAEVKKPGASVKVSCKASGYTFTSYHIQWVRQAPGQRLEWIGWIYPGDGSTQYNEKFKGRVTITRDTSASTAYMELSSLRSEDTAVYYCAREGTYYAMDYWGQGTLVTVSSGGGGSGGGGSGGGGSDVVMTQTPLSLSVTPGQPASISCRSSQSIVHSNGNTYLEWYLQKPGQSPQLLIYKVSNRFSGVPDRFSGSGSGTDFTLKISRVEAEDVGVYYCFQGSHVPRTFGGGTKVEIKTERRAEVPTAHPSPSPRPAGQFQTLVVGVVGGLLGSLVLLVWVLAVILRHRRQGKHWTSTQRKADFQHPAGAVGPEPTDRGLQWRSSPAADAQEEVTYAQLKHTQPEDGVEMDTRSPHDEDPQASIYATLKHSRPRREMASPPSPLSGEFLDTKDRQAEEDRQMDTEAAASEAPQDVTYAQLHSLTLRREATEPPPSQEGPSPAVPSIYATLAIH

SEQ ID NO: 332 - protein, 481 aa, synthetic construct
MALPVTALLLPLALLLHAARPQVQLVQSGAEVKKPGASVKVSCKASGYTFTSYHIQWVRQAPGQRLEWIGWIYPGDGSTQYNEKFKGRVTITRDTSASTAYMELSSLRSEDTAVYYCAREGTYYAMDYWGQGTLVTVSSGGGGSGGGGSGGGGSDVVMTQTPLSLSVTPGQPASISCRSSQSIVHSNGNTYLEWYLQKPGQSPQLLIYKVSNRFSGVPDRFSGSGSGTDFTLKISRVEAEDVGVYYCFQGSHVPRTFGGGTKVEIKTERRAEVPTAHPSPSPRPAGQFQTLVVGVVGGLLGSLVLLVWVLAVILRHRRQGKHWTSTQRKADFQHPAGAVGPEPTDRGLQWRSSPAADAQEETEYATIKHTQPEDGVEMDTRSPHDEDPQATEYATIKHSRPRREMASPPSPLSGEFLDTKDRQAEEDRQMDTEAAASEAPQDVTYAQLHSLTLRREATEPPPSQEGPSPAVPSIYATLAIH

SEQ ID NO: 333 - protein, 481 aa, synthetic construct
MALPVTALLLPLALLLHAARPQVQLVQSGAEVKKPGASVKVSCKASGYTFTSYHIQWVRQAPGQRLEWIGWIYPGDGSTQYNEKFKGRVTITRDTSASTAYMELSSLRSEDTAVYYCAREGTYYAMDYWGQGTLVTVSSGGGGSGGGGSGGGGSDVVMTQTPLSLSVTPGQPASISCRSSQSIVHSNGNTYLEWYLQKPGQSPQLLIYKVSNRFSGVPDRFSGSGSGTDFTLKISRVEAEDVGVYYCFQGSHVPRTFGGGTKVEIKTERRAEVPTAHPSPSPRPAGQFQTLVVGVVGGLLGSLVLLVWVLAVILRHRRQGKHWTSTQRKADFQHPAGAVGPEPTDRGLQWRSSPAADAQEEVTYAQLKHTQPEDGVEMDTRSPHDEDPQASIYATLKHSRPRREMASPPSPLSGEFLDTKDRQAEEDRQMDTEAAASEAPQDTEYATIHSLTLRREATEPPPSQEGPSPAVPTEYATIAIH

SEQ ID NO: 334 - protein, 481 aa, synthetic construct
MALPVTALLLPLALLLHAARPQVQLVQSGAEVKKPGASVKVSCKASGYTFTSYHIQWVRQAPGQRLEWIGWIYPGDGSTQYNEKFKGRVTITRDTSASTAYMELSSLRSEDTAVYYCAREGTYYAMDYWGQGTLVTVSSGGGGSGGGGSGGGGSDVVMTQTPLSLSVTPGQPASISCRSSQSIVHSNGNTYLEWYLQKPGQSPQLLIYKVSNRFSGVPDRFSGSGSGTDFTLKISRVEAEDVGVYYCFQGSHVPRTFGGGTKVEIKTERRAEVPTAHPSPSPRPAGQFQTLVVGVVGGLLGSLVLLVWVLAVILRHRRQGKHWTSTQRKADFQHPAGAVGPEPTDRGLQWRSSPAADAQEEVTYAQLKHTQPEDGVEMDTRSPHDEDPQATEYATIKHSRPRREMASPPSPLSGEFLDTKDRQAEEDRQMDTEAAASEAPQDTEYATIHSLTLRREATEPPPSQEGPSPAVPSIYATLAIH

(19) United States
(12) Patent Application Publication
(10) Pub. No.: US 2024/0382525 A1
(43) Pub. Date: Nov. 21, 2024

(54) CD4+ AND/OR CD8+ CELL POPULATIONS COMPRISING ICARS FOR USE IN TREATMENT THERAPIES

(71) Applicant: GAVISH-GALILEE BIO APPLICATIONS LTD., Kiryat Shmona (IL)

(72) Inventors: Michael WEIST, Kiryat Shmona (IL); Caitlin SCHNAIR, Kiryat Shmona (IL)

(73) Assignee: GAVISH-GALILEE BIO APPLICATIONS LTD., Kiryat Shmona (IL)

(21) Appl. No.: 18/704,224

(22) PCT Filed: Oct. 25, 2022
(86) PCT No.: PCT/US2022/078671

§ 371 Date: Apr. 24, 2024

Related U.S. Application Data
(60) Provisional application No. 63/271,924, filed on Oct. 26, 2021.

(51) Int. Cl. A61K 35/17; A61K 39/00; C07K 14/725; C12N 5/0783

(52) U.S. Cl. CPC A61K 35/17; A61K 39/4611; A61K 39/4631; A61K 39/464414; C07K 14/7051; C12N 5/0636; A61K 2239/13; A61K 2239/21; C07K 2317/622; C07K 2319/03

(57) ABSTRACT

The invention relates to the field of cancer immunotherapy by employing CD4+ cell populations, CD8+ cell populations, or a combination thereof, comprising bicistronic inhibitory chimeric antigen receptor (iCAR)/activating chimeric antigen receptor (aCAR) constructs for use in cancer treatment therapies.

### FIELD OF THE INVENTION

The invention relates to the field of cancer immunotherapy by employing CD4+ cell populations, CD8+ cell populations, and combinations thereof, comprising inhibitory chimeric antigen receptors (iCARs) paired with activating chimeric antigen receptors (aCARs) for use in cancer treatment therapies.

### BACKGROUND OF THE INVENTION

The identification of targetable antigens that are exclusively expressed by tumor cells but not by healthy tissue is undoubtedly the major challenge in cancer immunotherapy today. Clinical evidence that T cells are capable of eradicating tumor cells comes from numerous studies evaluating highly diverse approaches for harnessing T cells to treat cancer (Rosenberg and Restifo, Science, 348(6230): 62-68 (2015)). These approaches employ bone marrow transplantation with donor lymphocyte infusion, adoptive transfer of tumor-infiltrating lymphocytes (TILs), treatment with T cells genetically redirected at pre-selected antigens via CARs (Gross and Eshhar, Annual Review of Pharmacology and Toxicology, 56:59-83, (2016)) or T cell receptors (TCRs), the use of immune checkpoint inhibitors, BiTEs (bispecific T-cell engager molecules) technologies; Einsele, H., et al., Cancer, 126(14):3192-3201 (2020)), or active vaccination. Of these, the use of genetically engineered T cells and different strategies for active immunization entail pre-existing information on candidate antigens which are likely to exert a durable clinical response but minimal adverse effects. Yet, as stated in the title of a review by S. Rosenberg, “Finding suitable targets is the major obstacle to cancer gene therapy” (Rosenberg, Cancer Gene Therapy, 21:45-47 (2014))).

The concept of using chimeric antigen receptors (or CARs) to genetically redirect T cells (or other killer cells of the immune system such as natural killer (NK) cells and cytokine-induced killer cells) against antigens of choice in an MHC-independent manner was first introduced by Gross and Eshhar in the late 1980s (Gross et al., PNAS, 86(24):10024-1002 (1989). They are produced synthetically from chimeric genes encoding an extracellular single-chain antibody variable fragment (scFv) fused through a flexible hinge and transmembrane domain to costimulatory domains and signaling components comprising immunoreceptor tyrosine-based activation motifs of CD3-ζ or FcRγ chains capable of T cell activation. At present, CARs are being examined in dozens of clinical trials and have shown exceptionally high efficacy in B cell malignancies (Dotti et al., 2014; Gill and June, 263(1): 68-89 (2015)); Gross and Eshhar, Annual Review of Pharmacology and Toxicology, 56:59-83, 2016). The safety of CAR-T cell therapy is determined, in large part, by its ability to discriminate between the tumor and healthy tissue. A major risk in targeting solid tumors, and the direct cause for adverse autoimmune effects that have been reported in clinical and preclinical studies, is off-tumor, on-target toxicity resulting from extra-tumor expression of the target antigen (dealt with in detail in the review (Gross and Eshhar, 2016b) and (Klebanoff, et al., Nature Medicine 22:26-36 (2016)).

While undoubtedly intriguing, these previous CAR-based approaches require tuning the affinity of CAR scFv's to selectively bind high antigen levels in tumors while minimizing recognition of lower antigen levels in healthy tissues. In addition, the magnitude of both the activating and costimulatory signals needs to be balanced to allow effective on-target, on-tumor T cell reactivity. It is worth noting that in B cell malignancies, CARs targeted antigen exclusive to B cells and did not require titration of affinity or T cell signaling. For solid tumors, whether such balance can be routinely attained in the clinical setting is questionable.

Off-tumor reactivity occurs when the tumor antigen targeted by CAR-redirected killer cells is shared with normal tissue. However, if the normal tissue expresses another surface antigen that is not present on the tumor, it can be targeted by inhibitory CARs (iCARs) that contain an inhibitory signaling moiety which when engaged prevents T-cell activation by the activating CAR (aCAR). Co-expression of aCAR and iCAR will therefore direct killer cells to target tumors while sparing normal tissue.

Instead of an activating domain (such as FcRγ or CD3-ζ), an iCAR possesses a signaling domain derived from an inhibitory receptor which can antagonize T cell activation, such as CTLA-4, PD-1, or NK inhibitory receptors.

There remains a need in the art for cancer therapies, in particular therapies that comprise iCARs in order to limit off-target effects. The present invention meets that need by providing CD4+ cells, CD8+ cells, or a combination thereof, expressing aCAR and iCAR constructs which find use in cancer treatment.

### BRIEF SUMMARY OF THE INVENTION

The present invention provides a population of CD4+ cells, CD8+ cells, or a combination thereof, expressing a bicistronic inhibitory chimeric antigen receptor (iCAR)/activating chimeric antigen receptor (aCAR) construct or monocistronic aCAR and iCAR constructs for co-transduction comprising:
i. an iCAR portion, wherein the iCAR portion comprises:
   a. an iCAR single chain variable fragment (scFv) component optionally in the VH-VL or VL-VH orientation comprising a first linker, wherein the iCAR targets a first antigen;
   b. an iCAR hinge domain component;
   c. an iCAR transmembrane (TM) domain component;
   d. an iCAR inhibitory domain component; and
ii. an aCAR portion, wherein the iCAR portion comprises:
   a. an aCAR single chain variable fragment (scFv) component optionally in the VH-VL or VL-VH orientation comprising a second linker, wherein the aCAR scFv targets a second antigen;
   b. an aCAR hinge domain component;
   c. an aCAR transmembrane (TM) domain component;
   d. an aCAR co-stimulatory domain component
   e. an aCAR activation signaling domain; and
iii. the bicistronic or monocistronic constructs comprise a third linker that connects the iCAR portion in (i) and the aCAR portion in (ii).

In some embodiments, the first and/or second linker connecting the VH-VL or VL-VH in either orientation comprises one or more linker selected from the group consisting of (G4S)X3 linker (SEQ ID NO:81), G4S (SEQ ID NO:153), (G4S)X3 (SEQ ID NO:154), and Whitlow linker (SEQ ID NO:82).

In some embodiments, the iCAR scFv component targets an HLA antigen.

In some embodiments, the HLA antigen is selected from the group consisting of HLA-A2, HLA-A3, HLA-A, HLA-B, HLA-C, HLA-G, HLA-E, HLA-F, HLA-DPA1, HLA-DQA1, HLA-DQB1, HLA-DQB2, HLA-DRB1, and HLA-DRB5.

In some embodiments, the iCAR scFv component is selected from the group consisting of BB7.2, 3PF12, 3PF12/C4, 3PF12/F12, 3PF12/B11, W6/32, BBM.1, SN66E3, Ha5C2.A2, MWB1, MWB1-mod, Hz.BB7.2 VH1-69_A18VK, Hz.BB7.2 VH1-69 (27,30)_A18, Hz.BB7.2 VH1-69 (27,30,48)_A18, Hz.BB7.2 VH1-69 (27,30,67)_A18, Hz.BB7.2 VH1-69 (27,30,69) A18, Hz.BB7.2 VH1-69 (27,30,67,69)_A18, Hz.BB7.2 VH1-3_A18, Hz.BB7.2 VH1-3(48)_A18, Hz.BB7.2 VH1-3(67)_A18, Hz.BB7.2 VH1-3(69)_A18, Hz.BB7.2 VH1-3(71)_A18, Hz.BB7.2 VH1-3(73)_A18, MWB1.2, SN66E3.2 and SN66E3.3.

In some embodiments, the iCAR scFv component is BB7.2.

In some embodiments, the iCAR scFv comprises the Vh and Vl from BB7.2 (SEQ ID NOs: 37 and 38) or vhCDR1, vhCDR2, vhCDR3, vlCDR1, vlCDR2, and vlCDR3 from SEQ ID NOs: 37 and 38.

In some embodiments, the iCAR scFv comprises the Vh and Vl from Hz.BB7.2 VH1-69_A18VK (SEQ ID NOs: 57 and 58) or vhCDR1, vhCDR2, vhCDR3, vlCDR1, vlCDR2, and vlCDR3 from SEQ ID NOs: 57 and 58.

In some embodiments, the iCAR scFv comprises the Vh and Vl from Hz.BB7.2 VH1-69 (27,30)_A18 (SEQ ID NOs: 59 and 60) or vhCDR1, vhCDR2, vhCDR3, vlCDR1, vlCDR2, and vlCDR3 from SEQ ID NOs: 59 and 60.

In some embodiments, the iCAR scFv comprises the Vh and Vl from Hz.BB7.2 VH1-69 (27,30,48)_A18 (SEQ ID NOs: 61 and 62) or vhCDR1, vhCDR2, vhCDR3, vlCDR1, vlCDR2, and vlCDR3 from SEQ ID NOs: 61 and 62.

In some embodiments, the iCAR scFv comprises the Vh and Vl from Hz.BB7.2 VH1-69 (27,30,67)_A18 (SEQ ID NOs: 63 and 64) or vhCDR1, vhCDR2, vhCDR3, vlCDR1, vlCDR2, and vlCDR3 from SEQ ID NOs: 63 and 64.

In some embodiments, the iCAR scFv comprises the Vh and Vl from Hz.BB7.2 VH1-69 (27,30,69)_A18 (SEQ ID NOs: 65 and 66) or vhCDR1, vhCDR2, vhCDR3, vlCDR1, vlCDR2, and vlCDR3 from SEQ ID NOs: 65 and 66.

In some embodiments, the iCAR scFv comprises the Vh and Vl from Hz.BB7.2 VH1-69 (27,30,67,69) A18 (SEQ ID NOs: 67 and 68) or vhCDR1, vhCDR2, vhCDR3, vlCDR1, vlCDR2, and vlCDR3 from SEQ ID NOs: 67 and 68.

In some embodiments, the iCAR scFv comprises the Vh and Vl from Hz.BB7.2 VH1-3_A18 (SEQ ID NOs: 69 and 70) or vhCDR1, vhCDR2, vhCDR3, vlCDR1, vlCDR2, and vlCDR3 from SEQ ID NOs: 69 and 70.

In some embodiments, the iCAR scFv comprises the Vh and Vl from Hz.BB7.2 VH1-3(48) A18 (SEQ ID NOs: 71 and 72) or vhCDR1, vhCDR2, vhCDR3, vlCDR1, vlCDR2, and vlCDR3 from SEQ ID NOs: 71 and 72.

In some embodiments, the iCAR scFv comprises the Vh and Vl from Hz.BB7.2 VH1-3(67)_A18 (SEQ ID NOs: 73 and 74) or vhCDR1, vhCDR2, vhCDR3, vlCDR1, vlCDR2, and vlCDR3 from SEQ ID NOs: 73 and 74.

In some embodiments, the iCAR scFv comprises the Vh and Vl from Hz.BB7.2 VH1-3(69)_A18 (SEQ ID NOs: 75 and 76) or vhCDR1, vhCDR2, vhCDR3, vlCDR1, vlCDR2, and vlCDR3 from SEQ ID NOs: 75 and 76.

In some embodiments, the iCAR scFv comprises the Vh and Vl from Hz.BB7.2 VH1-3(71)_A18 (SEQ ID NOs: 77 and 78) or vhCDR1, vhCDR2, vhCDR3, vlCDR1, vlCDR2, and vlCDR3 from SEQ ID NOs: 77 and 78.

In some embodiments, the iCAR scFv comprises the Vh and Vl from Hz.BB7.2 VH1-3(73)_A18 (SEQ ID NOs: 79 and 80) or vhCDR1, vhCDR2, vhCDR3, vlCDR1, vlCDR2, and vlCDR3 from SEQ ID NOs: 79 and 80.

In some embodiments, the iCAR scFv is BB7.2 of SEQ ID NO:167.

In some embodiments, the iCAR scFv component is 3PF12.

In some embodiments, the iCAR scFv comprises the Vh and Vl from 3PF12/C4 (SEQ ID NOs: 39 and 40) or vhCDR1, vhCDR2, vhCDR3, vlCDR1, vlCDR2, and vlCDR3 from SEQ ID NOs: 39 and 40.

In some embodiments, the iCAR scFv comprises the Vh and Vl from 3PF12/F12 (SEQ ID NOs: 41 and 42) or vhCDR1, vhCDR2, vhCDR3, vlCDR1, vlCDR2, and vlCDR3 from SEQ ID NOs: 41 and 42.

In some embodiments, the iCAR scFv comprises the Vh and Vl from 3PF12/B11 (SEQ ID NOs: 43 and 44) or vhCDR1, vhCDR2, vhCDR3, vlCDR1, vlCDR2, and vlCDR3 from SEQ ID NOs: 43 and 44.

In some embodiments, the iCAR scFv is 3PF12 of SEQ ID NO:168.

In some embodiments, the iCAR scFv component is SN66E3.

In some embodiments, the iCAR scFv comprises the Vh and Vl from SN66E3.1 (SEQ ID NOs: 49 and 50) or vhCDR1, vhCDR2, vhCDR3, vlCDR1, vlCDR2, and vlCDR3 from SEQ ID NOs: 49 and 50.

In some embodiments, the iCAR scFv is SN66E3.1 of SEQ ID NO:169.

In some embodiments, the iCAR scFv comprises the Vh and Vl from SN66E3.2 (SEQ ID NOs: 165 and 166) or vhCDR1, vhCDR2, vhCDR3, vlCDR1, vlCDR2, and vlCDR3 from SEQ ID NOs: 165 and 166.

In some embodiments, the iCAR scFv is SN66E3.2 of SEQ ID NO:285.

In some embodiments, the iCAR scFv comprises the Vh and Vl from SN66E3.3 (SEQ ID NOs: 283 and 284) or vhCDR1, vhCDR2, vhCDR3, vlCDR1, vlCDR2, and vlCDR3 from SEQ ID NOs: 283 and 284.

In some embodiments, the iCAR scFv is SN66E3.3 of SEQ ID NO:286.

In some embodiments, the iCAR scFv component is W6/32.

In some embodiments, the iCAR scFv comprises the Vh and Vl from W6/32 (SEQ ID NOs: 45 and 46) or vhCDR1, vhCDR2, vhCDR3, vlCDR1, vlCDR2, and vlCDR3 from SEQ ID NOs: 45 and 46.

In some embodiments, the iCAR scFv component is BBM.1.

In some embodiments, the iCAR scFv comprises the Vh and Vl from BBM.1 (SEQ ID NOs: 47 and 48) or vhCDR1, vhCDR2, vhCDR3, vlCDR1, vlCDR2, and vlCDR3 from SEQ ID NOs: 47 and 48.

In some embodiments, the iCAR scFv component is Ha5C2.A2.

In some embodiments, the iCAR scFv comprises the Vh and Vl from Ha5C2.A2 (SEQ ID NOs: 51 and 52) or vhCDR1, vhCDR2, vhCDR3, vlCDR1, vlCDR2, and vlCDR3 from SEQ ID NOs: 51 and 52.

In some embodiments, the iCAR scFv component is MWB1.

In some embodiments, the iCAR scFv comprises the Vh and Vl from MWB1 (SEQ ID NOs: 53 and 54) or vhCDR1, vhCDR2, vhCDR3, vlCDR1, vlCDR2, and vlCDR3 from SEQ ID NOs: 53 and 54.

In some embodiments, the iCAR scFv comprises the Vh and Vl from MWB1-mod (MWB1.1) (SEQ ID NOs: 55 and 56) or vhCDR1, vhCDR2, vhCDR3, vlCDR1, vlCDR2, and vlCDR3 from SEQ ID NOs: 55 and 56.

In some embodiments, the iCAR scFv comprises the Vh and Vl from MWB1.2 (SEQ ID NOs: 163 and 164).

In some embodiments, the iCAR scFv is MWB1.1 scFvVH_VL (SEQ ID NO:273).

In some embodiments, the iCAR scFv is MWB1.2 scFvVH_VL (SEQ ID NO:274).

In some embodiments, the iCAR hinge domain component is selected from a PD-1 hinge, a CD28 hinge, and a CD8 hinge (including a CD8a hinge), a LIR1 Ig3-4 hinge, a LIR1 Ig-4 hinge, a LIR1 52 aa hinge, a LIR1 36 aa hinge, a LIR1 30 aa hinge, a LIR1 26 aa hinge, a LIR1 8 aa hinge, a CD33 hinge, a KIR2DL1 hinge, a PD-1 (47) hinge, a PD-1 (42) hinge, a PD-1 (36) hinge, a PD-1 (30) hinge, a PD-1 (26) hinge, and a PD-1 (20) hinge.

In some embodiments, the iCAR hinge domain component is a PD-1 hinge (SEQ ID NO:86).

In some embodiments, the iCAR hinge domain component is a CD28 hinge (SEQ ID NO:85).

In some embodiments, the iCAR hinge domain component is a CD8 alpha hinge (SEQ ID NO:84).

In some embodiments, the iCAR hinge domain component is a LIR1 Ig3-4 hinge (SEQ ID NO:87).

In some embodiments, the iCAR hinge domain component is a LIR1 Ig-4 hinge (SEQ ID NO:88).

In some embodiments, the iCAR hinge domain component is a LIR1 52 aa hinge (SEQ ID NO:89).

In some embodiments, the iCAR hinge domain component is a LIR1 36 aa hinge (SEQ ID NO:90).

In some embodiments, the iCAR hinge domain component is a LIR1 30 aa hinge (SEQ ID NO:91).

In some embodiments, the iCAR hinge domain component is a LIR1 26 aa hinge (SEQ ID NO:289).

In some embodiments, the iCAR hinge domain component is a LIR1 8 aa hinge (SEQ ID NO:92).

In some embodiments, the iCAR hinge domain component is a CD33 hinge (SEQ ID NO:93).

In some embodiments, the iCAR hinge domain component is a KIR2DL1 hinge (SEQ ID NO:94).

In some embodiments, the iCAR hinge domain component is a PD-1 (47) hinge (SEQ ID NO:290).

In some embodiments, the iCAR hinge domain component is a PD-1 (42) hinge (SEQ ID NO:291).

In some embodiments, the iCAR hinge domain component is a PD-1 (36) hinge (SEQ ID NO:292).

In some embodiments, the iCAR hinge domain component is a PD-1 (30) hinge (SEQ ID NO:293).

In some embodiments, the iCAR hinge domain component is a PD-1 (26) hinge (SEQ ID NO:294).

In some embodiments, the iCAR hinge domain component is a PD-1 (20) hinge (SEQ ID NO:295).

In some embodiments, the iCAR TM domain component is selected from a PD-1 TM domain, a CD28 TM domain, a CD8 TM domain (including a CD8a TM domain), a LIR1 TM domain, a CD33 TM domain, and a KIR2DL1 TM domain.

In some embodiments, the iCAR TM domain component is a PD-1 TM domain (SEQ ID NO:97).

In some embodiments, the iCAR TM domain component is a CD28 TM domain (SEQ ID NO:96).

In some embodiments, the iCAR TM domain component is a CD8 alpha TM domain (SEQ ID NO:95).

In some embodiments, the iCAR TM domain component is a LIR1 TM domain (SEQ ID NO:98).

In some embodiments, the iCAR TM domain component is a CD33 TM domain (SEQ ID NO:99).

In some embodiments, the iCAR TM domain component is a KIR2DL1 TM domain (SEQ ID NO:100).

In some embodiments, the iCAR inhibitory domain component is an inhibitory domain from a protein selected from the group consisting of PD-1, KIR2DL1, KIR2DL2, KIR2DL3, KIR2DL4, KIR2DL5A, KIR3DL1, KIR3DL2, KIR3DL3, LAIR1, CD22, CD33, SIGLEC5, SIGLEC6, SIGLEC7, SIGLEC8, SIGLEC9, SIGLEC10, SIGLEC11, SIGLEC12, PECAM1/CD31, CD200R1, FCRL1, FCRL2, FCRL3, FCRL4, FCRL5, SLAMF1, SLAMF5, BTLA, LAG3, 2B4, CD160, CEACAM1, TIM3, VISTA, TIGIT, SIRPalpha, FcγRIIB, CD5, CD300a, CD300f, LIR1, LIR2, LIR3, LIR5, LIR8, Ly9, 2×PD1(G4S), 2×PD1(PD1), PVRIg, and AA2AR.

In some embodiments, the iCAR inhibitory domain component is a PD-1 inhibitory domain (SEQ ID NO:101).

In some embodiments, the iCAR component is a KIR2DL1 inhibitory domain (SEQ ID NO:102).

In some embodiments, the iCAR component is a KIR2DL2 inhibitory domain (SEQ ID NO:103).

In some embodiments, the iCAR component is a KIR2DL3 inhibitory domain (SEQ ID NO:104).

In some embodiments, the iCAR inhibitory domain component is a KIR2DL4 inhibitory domain (SEQ ID NO:105).

In some embodiments, the iCAR inhibitory domain component is a KIR2DL5A inhibitory domain (SEQ ID NO:106).

In some embodiments, the iCAR inhibitory domain component is a KIR3DL1 inhibitory domain (SEQ ID NO:107).

In some embodiments, the iCAR inhibitory domain component is a KIR3DL2 inhibitory domain (SEQ ID NO:108).

In some embodiments, the iCAR inhibitory domain component is a KIR3DL3 inhibitory domain (SEQ ID NO:109).

In some embodiments, the iCAR inhibitory domain component is a LAIR1 inhibitory domain (SEQ ID NO:110).

In some embodiments, the iCAR inhibitory domain component is a CD22 inhibitory domain (SEQ ID NO:111).

In some embodiments, the iCAR inhibitory domain component is a CD33 inhibitory domain (SEQ ID NO:112).

In some embodiments, the iCAR inhibitory domain component is a SIGLEC5 inhibitory domain (SEQ ID NO:113).

In some embodiments, the iCAR inhibitory domain component is a SIGLEC6 inhibitory domain (SEQ ID NO:114).

In some embodiments, the iCAR inhibitory domain component is a SIGLEC7 inhibitory domain (SEQ ID NO:115).

In some embodiments, the iCAR inhibitory domain component is a SIGLEC8 inhibitory domain (SEQ ID NO:116).

In some embodiments, the iCAR inhibitory domain component is a SIGLEC9 inhibitory domain (SEQ ID NO:117).

In some embodiments, the iCAR inhibitory domain component is a SIGLEC10 inhibitory domain (SEQ ID NO:118).

In some embodiments, the iCAR inhibitory domain component is a SIGLEC11 inhibitory domain (SEQ ID NO:119).

In some embodiments, the iCAR inhibitory domain component is a SIGLEC12 inhibitory domain (SEQ ID NO:120).

In some embodiments, the iCAR inhibitory domain component is a PECAM1/CD31 inhibitory domain (SEQ ID NO:121).

In some embodiments, the iCAR inhibitory domain component is a CD200R1 inhibitory domain (SEQ ID NO:122).

In some embodiments, the iCAR inhibitory domain component is a FCRL1 inhibitory domain (SEQ ID NO:123).

In some embodiments, the iCAR inhibitory domain component is a FCRL2 inhibitory domain (SEQ ID NO:124).

In some embodiments, the iCAR inhibitory domain component is a FCRL3inhibitory domain (SEQ ID NO:125).

In some embodiments, the iCAR inhibitory domain component is a FCRL4 inhibitory domain (SEQ ID NO:126).

In some embodiments, the iCAR inhibitory domain component is a FCRL5 inhibitory domain (SEQ ID NO:127).

In some embodiments, the iCAR inhibitory domain component is a SLAMF1 inhibitory domain (SEQ ID NO:128).

In some embodiments, the iCAR inhibitory domain component is a SLAMF5 inhibitory domain (SEQ ID NO:129).

In some embodiments, the iCAR inhibitory domain component is a BTLA inhibitory domain (SEQ ID NO:130).

In some embodiments, the iCAR inhibitory domain component is a LAG3 inhibitory domain (SEQ ID NO:131).

In some embodiments, the iCAR inhibitory domain component is a 2B4 inhibitory domain (SEQ ID NO:132).

In some embodiments, the iCAR inhibitory domain component is a CD160 inhibitory domain (SEQ ID NO:133).

In some embodiments, the iCAR inhibitory domain component is a CEACAM1 inhibitory domain (SEQ ID NO:134).

In some embodiments, the iCAR inhibitory domain component is a TIM3 inhibitory domain (SEQ ID NO:135).

In some embodiments, the iCAR inhibitory domain component is a VISTA inhibitory domain (SEQ ID NO:136).

In some embodiments, the iCAR inhibitory domain component is a TIGIT inhibitory domain (SEQ ID NO:137).

In some embodiments, the iCAR inhibitory domain component is a SIRPalpha inhibitory domain (SEQ ID NO:138).

In some embodiments, the iCAR inhibitory domain component is a FcγRIIB inhibitory domain (SEQ ID NO:139).

In some embodiments, the iCAR inhibitory domain component is a CD5 inhibitory domain (SEQ ID NO:140).

In some embodiments, the iCAR inhibitory domain component is a CD300a inhibitory domain (SEQ ID NO:141).

In some embodiments, the iCAR inhibitory domain component is a CD300f inhibitory domain (SEQ ID NO:142).

In some embodiments, the iCAR inhibitory domain component is a LIR1 inhibitory domain (SEQ ID NO:143).

In some embodiments, the iCAR inhibitory domain component is a LIR2 inhibitory domain (SEQ ID NO:144).

In some embodiments, the iCAR inhibitory domain component is a LIR3 inhibitory domain (SEQ ID NO:145).

In some embodiments, the iCAR inhibitory domain component is a LIR5 inhibitory domain (SEQ ID NO:146).

In some embodiments, the iCAR inhibitory domain component is a LIR8 inhibitory domain (SEQ ID NO:147).

In some embodiments, the iCAR inhibitory domain component is a Ly9 inhibitory domain (SEQ ID NO:148).

In some embodiments, the iCAR inhibitory domain component is a 2×PD1(G4S) inhibitory domain (SEQ ID NO:149).

In some embodiments, the iCAR inhibitory domain component is a 2×PD1(PD1) inhibitory domain (SEQ ID NO:150).

In some embodiments, the iCAR inhibitory domain component is a PVRIg inhibitory domain (SEQ ID NO:151).

In some embodiments, the iCAR inhibitory domain component is a AA2AR inhibitory domain (SEQ ID NO:152).

In some embodiments, the aCAR single chain variable fragment (scFv) component targets Her2.

In some embodiments, the aCAR scFv comprises the Vh and Vl from trastuzumab (SEQ ID NOs:170 and 171, respectively).

In some embodiments, the aCAR scFv is SEQ ID NO:172.

In some embodiments, the aCAR scFv comprises the Vh and Vl from trastuzumab F9G (SEQ ID NOs: 307 and 308).

In some embodiments, the aCAR scFv comprises the Vh and Vl from pertuzumab (SEQ ID NOs:173 and 174, respectively).

In some embodiments, the aCAR scFv is SEQ ID NO:175.

130In some embodiments, the aCAR scFv comprises the Vh and Vl from FRP5 (SEQ ID NOs:176 and 177, respectively).

In some embodiments, the aCAR scFv comprises the Vh and Vl from A21 (SEQ ID NOs:178 and 179, respectively).

In some embodiments, the aCAR scFv comprises the Vh and Vl from XMT1517 (SEQ ID NOs:180 and 181, respectively).

In some embodiments, the aCAR scFv comprises the Vh and Vl from XMT1518 (SEQ ID NOs:182 and 183, respectively).

In some embodiments, the aCAR scFv comprises the Vh and Vl from XMT1519 (SEQ ID NOs:184 and 185, respectively).

In some embodiments, the aCAR scFv comprises the Vh and Vl from FWP51 (SEQ ID NOs:186 and 187, respectively).

In some embodiments, the aCAR scFv comprises SEQ ID NO:188.

In some embodiments, the aCAR single chain variable fragment (scFv) component targets EGFR.

In some embodiments, the aCAR scFv comprises the Vh and Vl from cetuximab (SEQ ID NOs:189 and 190, respectively).

In some embodiments, the aCAR scFv is SEQ ID NO:191.

In some embodiments, the aCAR scFv comprises the Vh and Vl from panitumumab (SEQ ID NOs:192 and 193, respectively).

In some embodiments, the aCAR scFv is SEQ ID NO:194.

In some embodiments, the aCAR scFv comprises the Vh and Vl from Imgatuzumab (SEQ ID NOs:195 and 196, respectively).

In some embodiments, the aCAR scFv comprises the Vh and Vl from Nimotuzumab (SEQ ID NOs:197 and 198, respectively).

In some embodiments, the aCAR scFv comprises the Vh and Vl from Nimotuzumab (K5) (SEQ ID NOs:310 and 311, respectively).

In some embodiments, the aCAR scFv comprises the Vh and Vl from Necitumumab (SEQ ID NOs:199 and 200, respectively).

In some embodiments, the aCAR scFv comprises the Vh and Vl from ICR62 (SEQ ID NOs:201 and 202, respectively).

In some embodiments, the aCAR scFv comprises the Vh and Vl from Matuzumab (SEQ ID NOs:204 and 205, respectively).

In some embodiments, the aCAR scFv comprises the Vh and Vl from C10 (SEQ ID NOs:206 and 207, respectively).

In some embodiments, the aCAR scFv comprises the Vh and Vl from Zalutumumab (SEQ ID NOs:208 and 209, respectively).

In some embodiments, the aCAR scFv comprises the Vh and Vl from P1X (SEQ ID NOs:210 and 211, respectively).

In some embodiments, the aCAR scFv comprises the Vh and Vl from P2X (SEQ ID NOs:212 and 213, respectively).

In some embodiments, the aCAR scFv comprises the Vh and Vl from P3X (SEQ ID NOs:214 and 215, respectively).

In some embodiments, the aCAR scFv comprises the VH from EGFR-1a1-VHH (SEQ ID NO:216).

In some embodiments, the aCAR scFv comprises the VH from EGFR-VHH (SEQ ID NO:312).

In some embodiments, the aCAR single chain variable fragment (scFv) component targets Mesothelin.

In some embodiments, the aCAR scFv comprise the Vh and Vl from Amatuximab (SEQ ID NOs:217 and 218, respectively).

In some embodiments, the aCAR scFv comprise the Vh and Vl from P4 (SEQ ID NOs:219 and 220, respectively).

In some embodiments, the aCAR scFv comprise the Vh and Vl from SS1 (SEQ ID NOs:222 and 223, respectively).

In some embodiments, the aCAR scFv comprise the VHH from SD1 (SEQ ID NO:225).

In some embodiments, the aCAR scFv comprise the VHH from SD2 (SEQ ID NO:226).

In some embodiments, the aCAR scFv comprise the Vh and Vl from 1H7 (SEQ ID NOs:227 and 228, respectively).

In some embodiments, the aCAR scFv comprise the Vh and Vl from 3C02 (SEQ ID NOs:230 and 231, respectively).

In some embodiments, the aCAR hinge TM domain component is selected from the group consisting of a CD28 hinge and a CD8 hinge (including a CD8a hinge domain).

In some embodiments, the aCAR hinge TM domain component is a CD28 hinge domain (SEQ ID NO:85).

In some embodiments, aCAR the hinge TM domain component is a CD8 alpha hinge domain (SEQ ID NO:84).

In some embodiments, the aCAR co-stimulatory domain component is selected from the group consisting of a CD137 (4-1BB) co-stimulatory domain, a CD28 co-stimulatory domain, a 28BB co-stimulatory domain, and a CD3z co-stimulatory domain.

In some embodiments, the aCAR co-stimulatory domain component is a CD137 (4-1BB) co-stimulatory domain (SEQ ID NO:233).

In some embodiments, the aCAR co-stimulatory domain component is a CD28 co-stimulatory domain (SEQ ID NO:234).

In some embodiments, the aCAR co-stimulatory domain component a CD3z activation signaling domain (SEQ ID NO:235).

In some embodiments, the aCAR ITAM is a CD3 zeta domain.

In some embodiments, the aCAR ITAM is a CD3 zeta domain (SEQ ID NO:236).

In some embodiments, the aCAR ITAM is a CD3 zeta 3F domain (SEQ ID NO:237).

In some embodiments, the aCAR ITAM is a CD3 zeta 4F domain (SEQ ID NO:238).

In some embodiments, the aCAR ITAM is a CD3 zeta 4OF domain (SEQ ID NO:239).

In some embodiments, the linker connecting the iCAR portion and the aCAR portion comprises one or more linker selected from the group consisting of T2A (SEQ ID NO:155), F2A (SEQ ID NO:156), P2A (SEQ ID NO:157), E2A (SEQ ID NO:158), and an IRES sequence (SEQ ID NO:159 or 160).

In some embodiments, the linker connecting the iCAR portion and the aCAR portion is GSG T2A (SEQ ID NO:155).

In some embodiments, the bicistronic iCAR/aCAR construct comprises an amino acid sequence encoded by a nucleic acid sequence selected from the group consisting of SEQ ID NO:1, SEQ ID NO:3, SEQ ID NO:5, SEQ ID NO:7, SEQ ID NO:9, SEQ ID NO:11, SEQ ID NO:13, SEQ ID NO:15, SEQ ID NO:17, SEQ ID NO:19, SEQ ID NO:21, SEQ ID NO:23, SEQ ID NO:25, SEQ ID NO:27, SEQ ID NO:29, SEQ ID NO:31, SEQ ID NO:33, SEQ ID NO:35, SEQ ID NO:275, SEQ ID NO:277, SEQ ID NO:279, SEQ ID NO:281, SEQ ID NO:321, SEQ ID NO:323, and SEQ ID NO:325.

In some embodiments, the bicistronic iCAR/aCAR construct comprises an amino acid sequence encoded by a nucleic acid sequence selected from the group consisting of SEQ ID NO:275, SEQ ID NO:277, SEQ ID NO:279, SEQ ID NO:281, SEQ ID NO:321, SEQ ID NO:323, and SEQ ID NO:325.

In some embodiments, the bicistronic iCAR/aCAR construct comprises an amino acid sequence selected from the group consisting of SEQ ID NO:2, SEQ ID NO:4, SEQ ID NO:6, SEQ ID NO:8, SEQ ID NO:10, SEQ ID NO:12, SEQ ID NO:14, SEQ ID NO:16, SEQ ID NO:18, SEQ ID NO:20, SEQ ID NO:22, SEQ ID NO:24, SEQ ID NO:26, SEQ ID NO:28, SEQ ID NO:30, SEQ ID NO:32, SEQ ID NO:34, SEQ ID NO:36, SEQ ID NO:276, SEQ ID NO:278, SEQ ID NO:280, SEQ ID NO:282, SEQ ID NO:322, SEQ ID NO:324, and SEQ ID NO:326.

In some embodiments, the bicistronic iCAR/aCAR construct comprises an amino acid sequence selected from the group consisting of SEQ ID NO:276, SEQ ID NO:278, SEQ ID NO:280, SEQ ID NO:282, SEQ ID NO:322, SEQ ID NO:324, and SEQ ID NO:326.

In some embodiments, the bicistronic iCAR/aCAR construct further comprises a short hairpin RNA (shRNA).

In some embodiments, the iCAR comprises a synthetic PD-1 or LIR1 sequence as shown in Table 8, including one selected from the group consisting of SEQ ID NO:243, SEQ ID NO:244, SEQ ID NO:245, SEQ ID NO:246, SEQ ID NO:247, SEQ ID NO:248, SEQ ID NO:249, SEQ ID NO:250, SEQ ID NO:251, SEQ ID NO:252, SEQ ID NO:253, SEQ ID NO:254, SEQ ID NO:296, SEQ ID NO:297, SEQ ID NO:298, SEQ ID NO:299, SEQ ID NO:300, SEQ ID NO:301, SEQ ID NO:302, and SEQ ID NO:304.

In some embodiments, the iCAR/aCAR comprises a construct as described in Table 1.

In some embodiments, the iCAR/aCAR comprises a nucleic acid sequence as described in Table 1, including SEQ ID NO:1, SEQ ID NO:3, SEQ ID NO:5, SEQ ID NO:7, SEQ ID NO:9, SEQ ID NO:11, SEQ ID NO:13, SEQ ID NO:15, SEQ ID NO:17, SEQ ID NO:19, SEQ ID NO:21, SEQ ID NO:23, SEQ ID NO:25, SEQ ID NO:27, SEQ ID NO:29, SEQ ID NO:31, SEQ ID NO:33, SEQ ID NO:35, SEQ ID NO:275, SEQ ID NO:277, SEQ ID NO:279, SEQ ID NO:281, SEQ ID NO:321, SEQ ID NO:323, and SEQ ID NO:325.

In some embodiments, the iCAR/aCAR comprises an amino acid sequence as described in Table 1, including SEQ ID NO:2, SEQ ID NO:4, SEQ ID NO:6, SEQ ID NO:8, SEQ ID NO:10, SEQ ID NO:12, SEQ ID NO:14, SEQ ID NO:16, SEQ ID NO:18, SEQ ID NO:20, SEQ ID NO:22, SEQ ID NO:24, SEQ ID NO:26, SEQ ID NO:28, SEQ ID NO:30, SEQ ID NO:32, SEQ ID NO:34, SEQ ID NO:36, SEQ ID NO:276, SEQ ID NO:278, SEQ ID NO:280, SEQ ID NO:282, SEQ ID NO:322, SEQ ID NO:324, and SEQ ID NO:326.

In some embodiments, the iCAR comprises an amino acid sequence selected from the group consisting of SEQ ID NO: 255, SEQ ID NO:256, SEQ ID NO:257, SEQ ID NO:258, SEQ ID NO:305, SEQ ID NO:259, SEQ ID NO:260, SEQ ID NO:261, SEQ ID NO:262, SEQ ID NO:263, SEQ ID NO:264, SEQ ID NO:265, SEQ ID NO:266, SEQ ID NO:267, SEQ ID NO:268, SEQ ID NO:269, SEQ ID NO:270, SEQ ID NO:271, SEQ ID NO:272, SEQ ID NO:327, SEQ ID NO:328, SEQ ID NO:329, SEQ ID NO:330, SEQ ID NO:331, SEQ ID NO:332, SEQ ID NO:333, and SEQ ID NO:334.

In some embodiments, the iCAR/aCAR comprises a construct as described in Table 1, Table 11 and/or Table 12.

In some embodiments, the iCAR/aCAR comprises a construct or portion thereof as described in any one of Tables 1 to 22.

In some embodiments, the aCAR comprises a construct as described in any one of Tables 15, 16, 17, and/or 21.

In some embodiments, the iCAR comprises a construct as described in any one of Tables 1, 2, 4, 9, 10, 11 and/or 12.

The present invention provides a population of CD4+ cells, CD8+ cells, or combination thereof, comprising a nucleic acid that encodes a bicistronic iCAR/aCAR construct or monocistronic aCAR and iCAR constructs for co-transduction described herein.

The present invention provides a population of CD4+ cells, CD8+ cells, or combination thereof, comprising a vector comprising a nucleic acid sequence encoding for a bicistronic iCAR/aCAR construct or monocistronic aCAR and iCAR constructs for co-transduction described herein.

In some embodiments, the iCAR/aCAR construct or monocistronic aCAR and iCAR constructs for co-transduction comprises a signal peptide upstream of the iCAR and/or aCAR portions.

In some embodiments, the signal peptide is a CD8 alpha signal peptide (SEQ ID NO: 161), a GM-CSF signal peptide (SEQ ID NO: 162), or a mIgK signal peptide (SEQ ID NO: 306).

In some embodiments, the cells demonstrate a decrease of about 20%, about 25%, about 30%, about 35%, about 40%, about 45%, about 50%, about 55%, about 60%, about 65%, about 70%, about 75%, about 80%, about 85%, about 90%, about 95%, or about 99% of IL-2 secretion when co-incubated with cells expressing both the first and second target antigens, as compared to IL-2 secretion when co-incubated with cells expressing only one of the first and second target antigens.

In some embodiments, the cells demonstrate a decrease of about 20%, about 25%, about 30%, about 35%, about 40%, about 45%, about 50%, about 55%, about 60%, about 65%, about 70%, about 75%, about 80%, about 85%, about 90%, about 95%, or about 99% of IFN-7 secretion when co-incubated with cells expressing both the first and second target antigens, as compared to IFN-7 secretion when co-incubated with cells expressing only one of the first and second target antigens.

In some embodiments, the cells demonstrate a decrease of about 20%, about 25%, about 30%, about 35%, about 40%, about 45%, about 50%, about 55%, about 60%, about 65%, about 70%, about 75%, about 80%, about 85%, about 90%, about 95%, or about 99% of TNFα secretion when co-incubated with cells expressing both the first and second target antigens, as compared to TNFα secretion when co-incubated with cells expressing only one of the first and second target antigens.

In some embodiments, the combination of comprises a ratio of CD4+ cells to CD8+ cells of about 20:1 to about 1:20, about 15:1 to about 1:15, about 10:1 to about 1:10, about 9:1 to about 1:9, about 8:1 to about 1:8, about 7:1 to about 1:7, about 6:1 to about 1:6, about 5:1 to about 1:5, about 4:1 to about 1:4, about 3:1 to about 1:3, about 2:1 to about 1:2, or about 1:1.

The present invention provides a method for treating cancer in a patient having a tumor characterized by LOH, comprising administering to the patient a population of CD4+ cells, CD8+ cells, or a combination thereof, described herein.

The present invention provides a method for treating cancer in a patient having a tumor characterized by a genetic mutation resulting in a complete loss of expression of a target gene or target extracellular polymorphic epitope gene, comprising administering to the patient a population of CD4+ cells, CD8+ cells, or a combination thereof, described herein.

The present invention provides a method for treating cancer in a patient having a tumor characterized by loss of heterozygosity (LOH), or other genetic loss or allelic imbalance phenotypes including, without limitation, loss of function or expression, resulting from mutations affecting one or more nucleotides, comprising administering to the patient a population of CD4+ cells, CD8+ cells, or a combination thereof, described herein.

In some embodiments, the cancer is selected from the group consisting of Acute Myeloid Leukemia [LAML], Adrenocortical carcinoma [ACC], Bladder Urothelial Carcinoma [BLCA], Brain Lower Grade Glioma [LGG], Breast invasive carcinoma [BRCA], Cervical squamous cell carcinoma and endocervical adenocarcinoma [CESC], Cholangiocarcinoma [CHOL], Colon adenocarcinoma [COAD], Esophageal carcinoma [ESCA], Glioblastoma multiforme [GBM], Head and Neck squamous cell carcinoma [HNSC], Kidney Chromophobe [KICH], Kidney renal clear cell carcinoma [KIRC], Kidney renal papillary cell carcinoma [KIRP], Liver hepatocellular carcinoma [LIHC], Lung adenocarcinoma [LUAD], Lung squamous cell carcinoma [LUSC], Lymphoid Neoplasm Diffuse Large B-cell Lymphoma [DLBC], Mesothelioma [MESO], Ovarian serous cystadenocarcinoma [OV], Pancreatic adenocarcinoma [PAAD], Pheochromocytoma and Paraganglioma [PCPG], Prostate adenocarcinoma [PRAD], Rectum adenocarcinoma [READ], Sarcoma [SARC], Skin Cutaneous Melanoma [SKCM], Stomach adenocarcinoma [STAD], Testicular Germ Cell Tumors [TGCT], Thymoma [THYM], Thyroid carcinoma [THCA], Uterine Carcinosarcoma [UCS], Uterine Corpus Endometrial Carcinoma [UCEC], Uveal Melanoma [UVM], Non-small cell lung carcinoma [NSCLC], and Small cell lung cancer [SCLC].

The present invention provides a method for treating an autoimmune disease in a patient in need thereof, comprising administering to the patient a population of CD4+ cells, CD8+ cell populations, or a combination thereof described herein.

The present invention provides a method for producing the population of CD4+ cells, described herein, the method comprising:
i. obtaining a population of effector immune cells directed to a tumor-associated antigen;
ii. enriching the effector immune cells for CD4+; and
iii. transfecting the CD4+ effector immune cells with a nucleic acid molecule or a vector comprising a nucleotide sequence encoding the bicistronic iCAR/aCAR construct or monocistronic aCAR and iCAR constructs for co-transduction.

The present invention provides a method for producing the population of CD4+ cells described herein, the method comprising:
i. obtaining a population of naïve effector immune cells;
ii. enriching the naïve effector immune cells for CD4+; and
iii. transfecting the CD4+ naïve effector immune cells with a nucleic acid molecule or a vector comprising a nucleotide sequence encoding the bicistronic iCAR/aCAR construct or monocistronic aCAR and iCAR constructs for co-transduction.

The present invention provides a method for producing the population of CD8+ cells described herein, the method comprising:
i. obtaining a population of effector immune cells directed to a tumor-associated antigen;
ii. enriching the effector immune cells for CD8+; and
iii. transfecting the CD8+ effector immune cells with a nucleic acid molecule or a vector comprising a nucleotide sequence encoding the bicistronic iCAR/aCAR construct or monocistronic aCAR and iCAR constructs for co-transduction.

The present invention provides a method for producing the population of CD8+ cells described herein, the method comprising:
i. obtaining a population of naïve effector immune cells;
ii. enriching the naïve effector immune cells for CD8+; and
iii. transfecting the CD8+ naïve effector immune cells with a nucleic acid molecule or a vector comprising a nucleotide sequence encoding the bicistronic iCAR/aCAR construct or monocistronic aCAR and iCAR constructs for co-transduction.

In some embodiments, step (ii) is performed before step (iii).

In some embodiments, step (iii) is performed before step (ii).

In some embodiments, the bicistronic iCAR/aCAR construct is encoded a single vector.

In some embodiments, the bicistronic iCAR and aCAR constructs are encoded on different/separate vectors.

In some embodiments, the monocistronic aCAR and iCAR constructs for co-transduction are encoded on a single vector.

In some embodiments, the monocistronic aCAR and iCAR constructs for co-transduction are encoded on different/separate vectors.

In some embodiments, the immune cell is a T-cell, a natural killer cell, or a cytokine-induced killer cell.

In some embodiments, the immune cell is a Jurkat T-cell, a Jurkat-NFAT T-cell, and/or a peripheral blood mononuclear cell (PBMC).

### Additional Embodiments

The following additional aspects and embodiments thereof described and illustrated below are meant to be exemplary and illustrative.

In one aspect, the present invention provides a population of CD4+ cells, CD8+ cells, or a combination thereof, comprising a bicistronic inhibitory chimeric antigen receptor (iCAR)/activating chimeric antigen receptor (aCAR) nucleotide construct which encodes:
i. an iCAR portion, comprising:
   a. an iCAR single chain variable fragment (scFv) component, optionally in the VH-VL or VL-VH orientation, comprising a first linker, wherein the iCAR targets a first antigen;
   b. an iCAR hinge domain component;
   c. an iCAR transmembrane (TM) domain component;
   d. an iCAR inhibitory domain component; and
ii. an aCAR portion, comprising:
   a. an aCAR single chain variable fragment (scFv) component, optionally in the VH-VL or VL-VH orientation, comprising a second linker, wherein the aCAR scFv targets a second antigen;
   b. an aCAR hinge domain component;
   c. an aCAR transmembrane (TM) domain component;
   d. an aCAR co-stimulatory domain component
   e. an aCAR activation signaling domain; and
iii. the bicistronic construct comprises a third linker that connects the iCAR portion in (i) and the aCAR portion in (ii).

In some embodiments, the first and/or second linker comprises one or more linkers selected from the group consisting of: (G4S)X3 linker (SEQ ID NO:81), G4S linker (SEQ ID NO:153), (G4S)X3 linker (SEQ ID NO:154), and Whitlow linker (SEQ ID NO:82).

In some embodiments, the iCAR scFv component targets an HLA antigen.

In some embodiments, the HLA antigen consists essentially of or is HLA-A2, HLA-A3, HLA-A, HLA-B, HLA-C, HLA-G, HLA-E, HLA-F, HLA-DPA1, HLA-DQA1, HLA-DQB1, HLA-DQB2, HLA-DRB1, and HLA-DRB5.

In some embodiments, the iCAR scFv component is selected from the group consisting of: BB7.2, 3PF12, 3PF12/C4, 3PF12/F12, 3PF12/B11, W6/32, BBM.1, SN66E3, Ha5C2.A2, MWB1, MWB1-mod, Hz.BB7.2 VH1-69_A18VK, Hz.BB7.2 VH1-69 (27,30)_A18, Hz.BB7.2 VH1-69 (27,30,48)_A18, Hz.BB7.2 VH1-69 (27,30,67)_A18, Hz.BB7.2 VH1-69 (27,30,69) A18, Hz.BB7.2 VH1-69 (27,30,67,69)_A18, Hz.BB7.2 VH1-3_A18, Hz.BB7.2 VH1-3(48)_A18, Hz.BB7.2 VH1-3(67)_A18, Hz.BB7.2 VH1-3(69)_A18, Hz.BB7.2 VH1-3(71)_A18, Hz.BB7.2 VH1-3(73)_A18, MWB1.2, SN66E3.2 and SN66E3.3.

In some embodiments, the iCAR scFv component comprises Hz BB7.2.1 (SEQ ID NO:287), or SN66E3.3 (SEQ ID NO:286).

In some embodiments, the iCAR scFv component consists essentially of or is: Hz BB7.2.1 (SEQ ID NO:287), or SN66E3.3 (SEQ ID NO:286).

In some embodiments, the iCAR scFv consists essentially of or is Hz BB7.2.1 (SEQ ID NO:287).

In some embodiments, the iCAR scFv consists essentially of or is SN66E3.3 (SEQ ID NO:286).

In some embodiments, the iCAR hinge domain component is selected from a LIR1 52 aa hinge, a LIR1 36 aa hinge, a LIR1 30 aa hinge, a LIR1 26 aa hinge, or a LIR1 8 aa hinge.

In some embodiments, the iCAR hinge domain component is selected from a LIR1 52 aa hinge, a LIR1 36 aa hinge, a LIR1 30 aa hinge, a LIR1 26 aa hinge, a LIR1 8 aa hinge.

In some embodiments, the iCAR inhibitory domain component is an inhibitory domain from a protein selected from the group consisting of LIR1, LIR2, LIR3, LIR5, or LIR8.

In some embodiments, the iCAR inhibitory domain component comprises, or consists essentially of, or is, a LIR1 inhibitory domain (SEQ ID NO:143).

In some embodiments, the iCAR inhibitory domain component comprises or consists of a LIR1 inhibitory domain (SEQ ID NO:143), or a sequence having at least 95% sequence identity thereto.

In some embodiments, the iCAR inhibitory domain component comprises or consists of a LIR1 inhibitory domain (SEQ ID NO:143), or a sequence having at least 96% sequence identity thereto.

In some embodiments, the iCAR inhibitor domain component comprises or consists of a LIR1 inhibitory domain (SEQ ID NO:143), or a sequence having at least 97% sequence identity thereto.

In some embodiments, the iCAR inhibitor domain component comprises or consists of a LIR1 inhibitory domain (SEQ ID NO:143), or a sequence having at least 98% sequence identity thereto.

In some embodiments, the iCAR inhibitor domain component comprises or consists of a LIR1 inhibitory domain (SEQ ID NO:143), or a sequence having at least 99% sequence identity thereto.

In some embodiments, the iCAR inhibitory domain component consists of a LIR1 inhibitory domain (SEQ ID NO:143), or a sequence having at least 95% sequence identity thereto.

In some embodiments, the iCAR inhibitory domain component consists of a LIR1 inhibitory domain (SEQ ID NO:143), or a sequence having at least 96% sequence identity thereto.

In some embodiments, the iCAR inhibitor domain component consists of a LIR1 inhibitory domain (SEQ ID NO:143), or a sequence having at least 97% sequence identity thereto.

In some embodiments, the iCAR inhibitor domain component consists of a LIR1 inhibitory domain (SEQ ID NO:143), or a sequence having at least 98% sequence identity thereto.

In some embodiments, the iCAR inhibitor domain component consists of a LIR1 inhibitory domain (SEQ ID NO:143), or a sequence having at least 99% sequence identity thereto.

In some embodiments, the iCAR inhibitory domain component is a LIR1 inhibitory domain (SEQ ID NO:143).

In some embodiments, the aCAR scFv comprises or consists of the VH and VL domains from trastuzumab (SEQ ID NOs:170 and 171, respectively).

In some embodiments, the aCAR scFv comprises or consists of the VH and VL domains from trastuzumab (SEQ ID NOs:170 and 171, respectively), in either the VH-VL orientation or the VL-VH orientation.

In some embodiments, the aCAR scFv consists essentially of the VH and VL domains from trastuzumab (SEQ ID NOs:170 and 171, respectively).

In some embodiments, the aCAR scFv consists essentially of the VH and VL domains from trastuzumab (SEQ ID NOs:170 and 171, respectively), in the VH-VL orientation.

In some embodiments, the aCAR scFv consists essentially of the VH and VL domains from trastuzumab (SEQ ID Nos:170 and 171, respectively), in the the VL-VH orientation.

In some embodiments, the aCAR scFv consists of the VH and VL domains from trastuzumab (SEQ ID Nos:170 and 171, respectively), in the the VH-VL orientation.

In some embodiments, the aCAR scFv consists of the VH and VL domains from trastuzumab (SEQ ID Nos:170 and 171, respectively), in the the VL-VH orientation.

In some embodiments, the aCAR scFv comprises or consists essentially of the VH and VL domains of SEQ ID NO:172.

In some embodiments, the aCAR scFv consists essentially of the VH and VL domains of SEQ ID NO:172, in the VH-VL orientation.

In some embodiments, the aCAR scFv consists essentially of the VH and VL domains of SEQ ID NO:172, in the VL-VH orientation.

In some embodiments, the aCAR scFv comprises or consists of the VH and VL domains of SEQ ID NO:172, in the VH-VL orientation.

In some embodiments, the aCAR scFv comprises or consists of the VH and VL domains of SEQ ID NO:172, in the VL-VH orientation.

In some embodiments, the aCAR hinge TM domain component comprises, or consists essentially of, or is a CD8 alpha hinge domain (SEQ ID NO:84).

In some embodiments, the aCAR hinge TM domain component consists essentially of, or is a CD8 alpha hinge domain (SEQ ID NO:84).

In some embodiments, the aCAR hinge TM domain component consists essentially of a CD8 alpha hinge domain (SEQ ID NO:84).

In some embodiments, the aCAR hinge TM domain component is a CD8 alpha hinge domain (SEQ ID NO:84).

In some embodiments, the aCAR hinge TM domain component is a CD8 alpha hinge domain (SEQ ID NO:84), or a sequence having at least 95%, 96%, 97%, 98% or 99% sequence identity thereto.

In some embodiments, the aCAR hinge TM domain component is a CD8 alpha hinge domain (SEQ ID NO:84), or a sequence having at least 95% sequence identity thereto.

In some embodiments, the aCAR hinge TM domain component is a CD8 alpha hinge domain (SEQ ID NO:84), or a sequence having at least 96% sequence identity thereto.

In some embodiments, the aCAR hinge TM domain component is a CD8 alpha hinge domain (SEQ ID NO:84), or a sequence having at least 97% sequence identity thereto.

In some embodiments, the aCAR hinge TM domain component is a CD8 alpha hinge domain (SEQ ID NO:84), or a sequence having at least 98% sequence identity thereto.

In some embodiments, the aCAR hinge TM domain component is a CD8 alpha hinge domain (SEQ ID NO:84), or a sequence having at least 99% sequence identity thereto.

In some embodiments, the aCAR co-stimulatory domain component is selected from the group consisting of a CD137 (4-1BB) co-stimulatory domain, a CD28 co-stimulatory domain, a 28BB co-stimulatory domain, and a CD3z co-stimulatory domain.

In some embodiments, the aCAR co-stimulatory domain component comprises a CD137 (4-1BB) co-stimulatory domain (SEQ ID NO:233) and a CD3z activation signaling domain (SEQ ID NO:235).

In some embodiments, the aCAR co-stimulatory domain component consists essentially of both: a CD137 (4-1BB) co-stimulatory domain (SEQ ID NO:233) and a CD3z activation signaling domain (SEQ ID NO:235).

In some embodiments, the aCAR co-stimulatory domain component consists of: a CD137 (4-1BB) co-stimulatory domain (SEQ ID NO:233) and a CD3z activation signaling domain (SEQ ID NO:235), or a sequence with at least 95%, 96%, 97%, 98% or 99% sequence identity thereto.

In some embodiments, the aCAR co-stimulatory domain component consists of: a CD137 (4-1BB) co-stimulatory domain (SEQ ID NO:233) and a CD3z activation signaling domain (SEQ ID NO:235), or a sequence with at least 95% sequence identity thereto.

In some embodiments, the aCAR co-stimulatory domain component consists of: a CD137 (4-1BB) co-stimulatory domain (SEQ ID NO:233) and a CD3z activation signaling domain (SEQ ID NO:235), or a sequence with at least 96% sequence identity thereto.

In some embodiments, the aCAR co-stimulatory domain component consists of: a CD137 (4-1BB) co-stimulatory domain (SEQ ID NO:233) and a CD3z activation signaling domain (SEQ ID NO:235), or a sequence with at least 97% sequence identity thereto.

In some embodiments, the aCAR co-stimulatory domain component consists of: a CD137 (4-1BB) co-stimulatory domain (SEQ ID NO:233) and a CD3z activation signaling domain (SEQ ID NO:235), or a sequence with at least 98% sequence identity thereto.

In some embodiments, the aCAR co-stimulatory domain component consists of: a CD137 (4-1BB) co-stimulatory domain (SEQ ID NO:233) and a CD3z activation signaling domain (SEQ ID NO:235), or a sequence with at least 99% sequence identity thereto.

In some embodiments, the aCAR co-stimulatory domain component comprises a CD137 (4-1BB) co-stimulatory domain (SEQ ID NO:233) and/or a CD3z activation signaling domain (SEQ ID NO:235).

In some embodiments, the aCAR co-stimulatory domain component consists essentially of: a CD137 (4-1BB) co-stimulatory domain (SEQ ID NO:233) and/or a CD3z activation signaling domain (SEQ ID NO:235).

In some embodiments, the aCAR co-stimulatory domain component consists of: a CD137 (4-1BB) co-stimulatory domain (SEQ ID NO:233) and/or a CD3z activation signaling domain (SEQ ID NO:235), or a sequence with at least 95%, 96%, 97%, 98% or 99% sequence identity thereto.

In some embodiments, the aCAR co-stimulatory domain component consists of: a CD137 (4-1BB) co-stimulatory domain (SEQ ID NO:233) and/or a CD3z activation signaling domain (SEQ ID NO:235), or a sequence with at least 95% sequence identity thereto.

In some embodiments, the aCAR co-stimulatory domain component consists of: a CD137 (4-1BB) co-stimulatory domain (SEQ ID NO:233) and/or a CD3z activation signaling domain (SEQ ID NO:235), or a sequence with at least 96% sequence identity thereto.

In some embodiments, the aCAR co-stimulatory domain component consists of: a CD137 (4-1BB) co-stimulatory domain (SEQ ID NO:233) and/or a CD3z activation signaling domain (SEQ ID NO:235), or a sequence with at least 97% sequence identity thereto.

In some embodiments, the aCAR co-stimulatory domain component consists of: a CD137 (4-1BB) co-stimulatory domain (SEQ ID NO:233) and/or a CD3z activation signaling domain (SEQ ID NO:235), or a sequence with at least 98% sequence identity thereto.

In some embodiments, the aCAR co-stimulatory domain component consists of: a CD137 (4-1BB) co-stimulatory domain (SEQ ID NO:233) and/or a CD3z activation signaling domain (SEQ ID NO:235), or a sequence with at least 99% sequence identity thereto.

In some embodiments, the aCAR co-stimulatory domain component comprises a CD137 (4-1BB) co-stimulatory domain (SEQ ID NO:233).

In some embodiments, the aCAR co-stimulatory domain component consists essentially of or is a CD137 (4-1BB) co-stimulatory domain (SEQ ID NO:233).

In some embodiments, the aCAR co-stimulatory domain component consists essentially of a CD137 (4-1BB) co-stimulatory domain (SEQ ID NO:233).

In some embodiments, the aCAR co-stimulatory domain component is a CD137 (4-1BB) co-stimulatory domain (SEQ ID NO:233).

In some embodiments, the aCAR co-stimulatory domain component is a CD137 (4-1BB) co-stimulatory domain (SEQ ID NO:233), or a sequence with at least 95%, 96%, 97%, 98% or 99% sequence identity thereto.

In some embodiments, the aCAR co-stimulatory domain component is a CD137 (4-1BB) co-stimulatory domain (SEQ ID NO:233), or a sequence with at least 95% sequence identity thereto.

In some embodiments, the aCAR co-stimulatory domain component is a CD137 (4-1BB) co-stimulatory domain (SEQ ID NO:233), or a sequence with at least 96% sequence identity thereto.

In some embodiments, the aCAR co-stimulatory domain component is a CD137 (4-1BB) co-stimulatory domain (SEQ ID NO:233), or a sequence with at least 97% sequence identity thereto.

In some embodiments, the aCAR co-stimulatory domain component is a CD137 (4-1BB) co-stimulatory domain (SEQ ID NO:233), or a sequence with at least 98% sequence identity thereto.

In some embodiments, the aCAR co-stimulatory domain component is a CD137 (4-1BB) co-stimulatory domain (SEQ ID NO:233), or a sequence with at least 99% sequence identity thereto.

In some embodiments, the aCAR co-stimulatory domain component comprises a CD3z activation signaling domain (SEQ ID NO:235).

In some embodiments, the aCAR co-stimulatory domain consists essentially of or is a CD3z activation signaling domain (SEQ ID NO:235).

In some embodiments, the aCAR co-stimulatory domain consists essentially of a CD3z activation signaling domain (SEQ ID NO:235), or a sequence with at least 95%, 96%, 97%, 98% or 99% sequence identity thereto.

In some embodiments, the aCAR co-stimulatory domain is a CD3z activation signaling domain (SEQ ID NO:235).

In some embodiments, the aCAR co-stimulatory domain consists of a CD3z activation signaling domain (SEQ ID NO:235), or a sequence with at least 95%, 96%, 97%, 98% or 99% sequence identity thereto.

In some embodiments, the aCAR co-stimulatory domain consists of a CD3z activation signaling domain (SEQ ID NO:235), or a sequence with at least 98% sequence identity thereto.

In some embodiments, the aCAR co-stimulatory domain consists of a CD3z activation signaling domain (SEQ ID NO:235), or a sequence with at least 99% sequence identity thereto.

In some embodiments, the linker connecting the iCAR portion and the aCAR portion is encoded by a nucleotide sequence that comprises, or consists essentially of, or is: a T2A sequence (SEQ ID NO:155) and/or an IRES sequence (SEQ ID NO:159 or 160).

In some embodiments, the linker connecting the iCAR portion and the aCAR portion is encoded by a nucleotide sequence that comprises, or consists essentially of, or is: a T2A sequence (SEQ ID NO:155) or an IRES sequence (SEQ ID NO:159 or 160).

In some embodiments, the linker connecting the iCAR portion and the aCAR portion is encoded by a nucleotide sequence that comprises or consists of: a T2A sequence (SEQ ID NO:155) or an IRES sequence (SEQ ID NO:159 or 160).

In some embodiments, the linker connecting the iCAR portion and the aCAR portion is encoded by a nucleotide sequence that consists of: a T2A sequence (SEQ ID NO:155) or an IRES sequence (SEQ ID NO:159 or 160).

In some embodiments, the linker connecting the iCAR portion and the aCAR portion is encoded by a nucleotide sequence that comprises or consists of: an IRES sequence (SEQ ID NO:159 or 160).

In some embodiments, the linker connecting the iCAR portion and the aCAR portion is encoded by a nucleotide sequence that consists of: an IRES sequence (SEQ ID NO:159 or 160).

In some embodiments, the linker connecting the iCAR portion and the aCAR portion is encoded by a nucleotide sequence that consists essentially of: a T2A sequence (SEQ ID NO:155), or a sequence with at least 95%, 96%, 97%, 98% or 99% sequence identity thereto.

In some embodiments, the linker connecting the iCAR portion and the aCAR portion is encoded by a nucleotide sequence that consists of: a T2A sequence (SEQ ID NO:155), or a sequence with at least 95%, 96%, 97%, 98% or 99% sequence identity thereto.

In some embodiments, the linker connecting the iCAR portion and the aCAR portion is encoded by a nucleotide sequence that consists essentially of: an IRES sequence (SEQ ID NO:159 or 160), or a sequence with at least 95%, 96%, 97%, 98% or 99% sequence identity thereto.

In some embodiments, the linker connecting the iCAR portion and the aCAR portion is encoded by a nucleotide sequence that consists of: an IRES sequence (SEQ ID NO:159 or 160), or a sequence with at least 95%, 96%, 97%, 98% or 99% sequence identity thereto.

In some embodiments, the linker connecting the iCAR portion and the aCAR portion is encoded by a nucleotide sequence that comprises, or consists essentially of, or is, an IRES sequence (SEQ ID NO: 159).

In some embodiments, the linker connecting the iCAR portion and the aCAR portion is encoded by a nucleotide sequence that comprises, or consists essentially of, an IRES sequence (SEQ ID NO: 159).

In some embodiments, the linker connecting the iCAR portion and the aCAR portion is encoded by a nucleotide sequence that comprises an IRES sequence (SEQ ID NO: 159).

In some embodiments, the linker connecting the iCAR portion and the aCAR portion is encoded by a nucleotide sequence that consists essentially of: an IRES sequence (SEQ ID NO:159).

In some embodiments, the linker connecting the iCAR portion and the aCAR portion is encoded by a nucleotide sequence that consists essentially of: an IRES sequence (SEQ ID NO:159), or a sequence with at least 95%, 96%, 97%, 98% or 99% sequence identity thereto.

In some embodiments, the linker connecting the iCAR portion and the aCAR portion is encoded by a nucleotide sequence that consists essentially of: an IRES sequence (SEQ ID NO:159), or a sequence with at least 98% sequence identity thereto.

In some embodiments, the linker connecting the iCAR portion and the aCAR portion is encoded by a nucleotide sequence that consists essentially of: an IRES sequence (SEQ ID NO:159), or a sequence with at least 99% sequence identity thereto.

In some embodiments, the linker connecting the iCAR portion and the aCAR portion is encoded by a nucleotide sequence that consists of: an IRES sequence (SEQ ID NO:159), or a sequence with at least 95%, 96%, 97%, 98% or 99% sequence identity thereto.

In some embodiments, the linker connecting the iCAR portion and the aCAR portion is encoded by a nucleotide sequence that consists of: an IRES sequence (SEQ ID NO:159), or a sequence with at least 95% sequence identity thereto.

In some embodiments, the linker connecting the iCAR portion and the aCAR portion is encoded by a nucleotide sequence that consists of: an IRES sequence (SEQ ID NO:159), or a sequence with at least 96% sequence identity thereto.

In some embodiments, the linker connecting the iCAR portion and the aCAR portion is encoded by a nucleotide sequence that consists of: an IRES sequence (SEQ ID NO:159), or a sequence with at least 97% sequence identity thereto.

In some embodiments, the linker connecting the iCAR portion and the aCAR portion is encoded by a nucleotide sequence that consists of: an IRES sequence (SEQ ID NO:159), or a sequence with at least 98% sequence identity thereto.

In some embodiments, the linker connecting the iCAR portion and the aCAR portion is encoded by a nucleotide sequence that consists of: an IRES sequence (SEQ ID NO:159), or a sequence with at least 99% sequence identity thereto.

In some embodiments, the bicistronic iCAR/aCAR construct comprises, or consists essentially of, or is, the nucleic acid sequence of SEQ ID NO:277.

In some embodiments, the bicistronic iCAR/aCAR construct comprises or consists essentially of the nucleic acid sequence of SEQ ID NO:277.

In some embodiments, the bicistronic iCAR/aCAR construct consists essentially of the nucleic acid sequence of SEQ ID NO:277.

In some embodiments, the bicistronic iCAR/aCAR construct consists essentially of the nucleic acid sequence of SEQ ID NO:277, or a sequence with at least 98% sequence identity thereto.

In some embodiments, the bicistronic iCAR/aCAR construct consists essentially of the nucleic acid sequence of SEQ ID NO:277, or a sequence with at least 99% sequence identity thereto.

In some embodiments, the bicistronic iCAR/aCAR construct is the nucleic acid sequence of SEQ ID NO:277.

In some embodiments, the bicistronic iCAR/aCAR construct is the nucleic acid sequence of SEQ ID NO:277, or a sequence with at least 98% sequence identity thereto.

In some embodiments, the bicistronic iCAR/aCAR construct is the nucleic acid sequence of SEQ ID NO:277, or a sequence with at least 99% sequence identity thereto.

In some embodiments, the bicistronic iCAR/aCAR construct comprises, or consists essentially of, or is, the nucleic acid sequence of SEQ ID NO:279.

In some embodiments, the bicistronic iCAR/aCAR construct comprises or consists essentially of the nucleic acid sequence of SEQ ID NO:279.

In some embodiments, the bicistronic iCAR/aCAR construct consists essentially of the nucleic acid sequence of SEQ ID NO:279.

In some embodiments, the bicistronic iCAR/aCAR construct consists essentially of the nucleic acid sequence of SEQ ID NO:279, or a sequence with at least 98% sequence identity thereto.

In some embodiments, the bicistronic iCAR/aCAR construct consists essentially of the nucleic acid sequence of SEQ ID NO:279, or a sequence with at least 99% sequence identity thereto.

In some embodiments, the bicistronic iCAR/aCAR construct is the nucleic acid sequence of SEQ ID NO:279.

In some embodiments, the bicistronic iCAR/aCAR construct is the nucleic acid sequence of SEQ ID NO:279, or a sequence with at least 98% sequence identity thereto.

In some embodiments, the bicistronic iCAR/aCAR construct is the nucleic acid sequence of SEQ ID NO:279, or a sequence with at least 99% sequence identity thereto.

In some embodiments, the bicistronic iCAR/aCAR construct further comprises or consists essentially of: a nucleotide sequence as set forth in one or more of: SEQ ID NO:240, SEQ ID NO:241 or SEQ ID NO:242.

In some embodiments, the bicistronic iCAR/aCAR construct further consists of: a nucleotide sequence as set forth in one or more of: SEQ ID NO:240, SEQ ID NO:241 or SEQ ID NO:242.

In some embodiments, the bicistronic iCAR/aCAR construct further consists of: a nucleotide sequence as set forth in at least one of: SEQ ID NO:240, SEQ ID NO:241 or SEQ ID NO:242.

In some embodiments, the bicistronic iCAR/aCAR construct further consists of: a nucleotide sequence as set forth in at least one of: SEQ ID NO:240, SEQ ID NO:241 or SEQ ID NO:242, or a nucleotide sequence with at least 98% sequence identity thereto.

In some embodiments, the bicistronic iCAR/aCAR construct further consists of: a nucleotide sequence as set forth in at least one of: SEQ ID NO:240, SEQ ID NO:241 or SEQ ID NO:242, or a nucleotide sequence with at least 99% sequence identity thereto.

In some embodiments, the bicistronic iCAR/aCAR construct further consists of: a nucleotide sequence as set forth in: SEQ ID NO:240.

In some embodiments, the bicistronic iCAR/aCAR construct further consists of: a nucleotide sequence as set forth in: SEQ ID NO:240, or a nucleotide sequence with at least 99% sequence identity thereto.

In some embodiments, the bicistronic iCAR/aCAR construct further consists of: a nucleotide sequence as set forth in: SEQ ID NO:241.

In some embodiments, the bicistronic iCAR/aCAR construct further consists of: a nucleotide sequence as set forth in: SEQ ID NO:241, or a nucleotide sequence with at least 99% sequence identity thereto.

In some embodiments, the bicistronic iCAR/aCAR construct further consists of: a nucleotide sequence as set forth in: SEQ ID NO:242.

In some embodiments, the bicistronic iCAR/aCAR construct further consists of: a nucleotide sequence as set forth in: SEQ ID NO:242, or a nucleotide sequence with at least 99% sequence identity thereto.

In some embodiments, the bicistronic iCAR/aCAR construct further comprises a nucleotide sequence that encodes a CD8 alpha signal peptide as set forth in (SEQ ID NO: 161).

In some embodiments, the bicistronic iCAR/aCAR construct further consists essentially of a nucleotide sequence that encodes a CD8 alpha signal peptide as set forth in (SEQ ID NO: 161).

In some embodiments, the bicistronic iCAR/aCAR construct further consists essentially of a nucleotide sequence that encodes a CD8 alpha signal peptide as set forth in (SEQ ID NO: 161), or a sequence with at least 98% sequence identity thereto.

In some embodiments, the bicistronic iCAR/aCAR construct further consists essentially of a nucleotide sequence that encodes a CD8 alpha signal peptide as set forth in (SEQ ID NO: 161), or a sequence with at least 99% sequence identity thereto.

In some embodiments, the bicistronic iCAR/aCAR construct further consists of a nucleotide sequence that encodes a CD8 alpha signal peptide as set forth in (SEQ ID NO: 161).

In some embodiments, the bicistronic iCAR/aCAR construct further consists of a nucleotide sequence that encodes a CD8 alpha signal peptide as set forth in (SEQ ID NO: 161), or a sequence with at least 98% sequence identity thereto.

In some embodiments, the bicistronic iCAR/aCAR construct further consists of a nucleotide sequence that encodes a CD8 alpha signal peptide as set forth in (SEQ ID NO: 161), or a sequence with at least 99% sequence identity thereto.

### BRIEF DESCRIPTION OF THE DRAWINGS

FIG. 1 shows bicistronic construct design overview and component table.

FIG. 2A-2H show bicistronic survey—constructs MC0280-MC0300, MC0428, MC0447, MC0449, HLA-A2 shRNA.

FIG. 3A-3B shows a schematic for IMPT001: A dual CART system designed to kill based on tumor specific loss-of-HLA-A2 gene expression.

FIG. 4 shows efficacy analysis of untouched, CD4, and CD8 CAR T cells comprising a VR33 aCAR construct. Both A2+ target cells and A2− target cells were killed in a similar manner.

FIG. 5 shows protection of A2+ target cell line (H1703 WT) (top panels) and efficacy of A2− target cell line (H1703 KO) (bottom panels) analysis of untouched, CD4, and CD8 CAR T cells comprising VR51 and VR354 bicistronic iCAR/aCAR constructs. CD4+ cells maintained similar efficacy and showed complete protection.

FIG. 6 shows protection of A2+ target cell line (H1703 WT) (left panel) and efficacy of A2− target cell line (H1703 KO) (right panel) analysis of thawed untouched, CD4, and CD8 CAR T cells comprising a VR54 bicistronic iCAR/aCAR construct. Thawed CD4+ cells maintained similar efficacy and showed complete protection.

FIG. 7 shows protection analysis of of A2+ target cell line (H1650 WT) untouched (UT), CD4, and CD8 CAR T cells isolated on Day 0 or Day 14 and comprising a VR33 aCAR construct (33E) or a VR354 bicistronic iCAR/aCAR construct (354E). Day0 and Day14 CD4+ cells maintained similar efficacy and showed complete protection.

FIG. 8 shows protection of A2+ target cell line (H1703 WT) (top panels) and efficacy of A2− target cell line (H1703 KO) (bottom panels) analysis of untouched, CD4, and CD8 CAR T cells comprising a VR33 aCAR construct (33E), a VR354 bicistronic iCAR/aCAR construct (354E), or a VR449 bicistronic iCAR/aCAR construct (449E).

FIG. 9 shows FACS analysis of CD4+ and CD8+ CAR T cells following negative selection.

FIG. 10 shows FACS analysis of aCAR-iCAR expression in isolated CD4 and CD8 cells.

FIG. 11 shows analysis of IFNg secretion of untouched, CD4, and CD8 CAR T cells following co-incubation with A2+ or A2− target cell lines. For both CD4+ and CD8+ protection was complete, as represented by no IFNg secretion.

FIG. 12 shows analysis of killing and IFNg secretion following co-incubation with A2+ and A2− target cells by VR33 untouched cells.

FIG. 13 shows analysis of killing and IFNg secretion following co-incubation with A2+ and A2− target cells by VR51 untouched cells.

FIG. 14 shows analysis of killing and IFNg secretion following co-incubation with A2+ and A2− target cells by VR354 untouched cells.

FIG. 15 shows analysis of killing and IFNg secretion following co-incubation with A2+ and A2− target cells by VR33 CD4 cells.

FIG. 16 shows analysis of killing and IFNg secretion following co-incubation with A2+ and A2− target cells by VR51 CD4 cells.

FIG. 17 shows analysis of killing and IFNg secretion following co-incubation with A2+ and A2− target cells by VR354 CD4 cells.

FIG. 18 shows analysis of killing and IFNg secretion following co-incubation with A2+ and A2− target cells by VR33 CD8 cells.

FIG. 19 shows analysis of killing and IFNg secretion following co-incubation with A2+ and A2− target cells by VR51 CD8 cells.

FIG. 20 shows analysis of killing and IFNg secretion following co-incubation with A2+ and A2− cells by VR354 CD8 cells.

FIG. 21 shows analysis of IL2 production for untouched, CD4, and CD8 CAR T cells following co-incubation with A2+ and A2− target cells.

FIG. 22 shows analysis of IL4 production for untouched, CD4, and CD8 CAR T cells following co-incubation with A2+ and A2− target cells.

### DETAILED DESCRIPTION OF THE INVENTION

### I. Introduction

The present invention provides CD4+ cell populations, CD8+ cell populations, or a combination thereof, comprising bicistronic and co-administered monocistronic constructs specifically targeting tumor cells while keeping the normal cells protected. The cells provided herein comprise iCAR/aCAR constructs provided herein that target single allelic variants of polymorphic cell surface epitopes, which are lost from tumor cells due to loss of heterozygosity (LOH) of the chromosomal region they reside in, while remaining expressed on normal tissue. Because of the polymorphic variation, the iCAR/aCAR pair present in the CD4+ cells, CD8+ cells, or a combination thereof, is able to distinguish the two alleles and target only the tumor cells missing the target allele due to LOH.

### II. Select Definitions

The term “nucleic acid molecule” as used herein refers to a DNA or RNA molecule.

The term “encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (e.g., rRNA, tRNA and mRNA) or a defined sequence of amino acids and the biological properties resulting therefrom. Thus, a gene encodes a protein if transcription and translation of mRNA corresponding to that gene produces the protein in a cell or other biological system. Both the coding strand, the nucleotide sequence of which is identical to the mRNA sequence and is usually provided in sequence listings, and the non-coding strand, used as the template for transcription of a gene or cDNA, can be referred to as encoding the protein or other product of that gene or cDNA.

Unless otherwise specified, a “nucleotide sequence encoding an amino acid sequence” includes all nucleotide sequences that are degenerate versions of each other and that encode the same amino acid sequence. Nucleotide sequences that encode proteins and RNA may include introns.

The term “endogenous” refers to any material from or produced inside an organism, cell, tissue or system.

The term “exogenous” refers to any material introduced from or produced outside an organism, cell, tissue or system.

The term “expression” as used herein is defined as the transcription and/or translation of a particular nucleotide sequence driven by its promoter.

“Expression vector” refers to a vector comprising a recombinant polynucleotide comprising expression control sequences operatively linked to a nucleotide sequence to be expressed. An expression vector comprises sufficient cis-acting elements for expression; other elements for expression can be supplied by the host cell or in an in vitro expression system. Expression vectors include all those known in the art, such as cosmids, plasmids (e.g., naked or contained in liposomes) and viruses (e.g., lentiviruses, retroviruses, adenoviruses, and adeno-associated viruses) that incorporate the recombinant polynucleotide.

The term “genomic variant” as used herein refers to a change of at least one nucleotide at the genomic level in a sequenced sample compared to the reference or consensus sequence at the same genomic position.

The term “corresponding reference allele” as used herein with reference to a variant means the reference or consensus sequence or nucleotide at the same genomic position as the variant.

The term “extracellular domain” as used herein with reference to a protein means a region of the protein which is outside of the cell membrane.

The term “loss of heterozygosity” or “LOH” as used herein means the loss of chromosomal materials such as a complete chromosome or a part thereof, in one copy of the two chromosomes in a somatic cell.

The term “sequence region” as used herein with reference to a variant or a reference allele means a sequence starting upstream and ending downstream from the position of the variant, which can be translated into an “epitope peptide” that can be recognized by an antibody.

The term “CAR”, as that term is used herein, refers to a chimeric polypeptide that shares structural and functional properties with a cell immune-function receptor or adaptor molecule, from e.g., a T cell or a NK cell. CARs include TCARs and NKR-CARs. Upon binding to cognate antigen, a CAR can activate or inactivate the cytotoxic cell in which it is disposed, or modulate the cell's antitumor activity or otherwise modulate the cells immune response.

The term “specific binding” as used herein in the context of an extracellular domain, such as an scFv, that specifically binds to a single allelic variant of a polymorphic cell surface epitope, refers to the relative binding of the scFv to one allelic variant and its failure to bind to the corresponding different allelic variant of the same polymorphic cell surface epitope. Since this depends on the avidity (number of CAR copies on the T cell, number of antigen molecules on the surface of target cells (or cells to be protected) and the affinity of the specific CARs used, a functional definition would be that the specific scFv would provide a significant signal in an ELISA against the single allelic variant of a polymorphic cell surface epitope to which it is specific or cells transfected with a CAR displaying the scFv would be clearly labeled with the single allelic variant of a polymorphic cell surface epitope in a FACS assay, while the same assays using the corresponding different allelic variant of the same polymorphic cell surface epitope would not give any detectable signal.

The term “treating” as used herein refers to means of obtaining a desired physiological effect. The effect may be therapeutic in terms of partially or completely curing a disease and/or symptoms attributed to the disease. The term refers to inhibiting the disease, e.g., arresting its development; or ameliorating the disease, e.g., causing regression of the disease.

As used herein, the terms “subject” or “individual” or “animal” or “patient” or “mammal,” refers to any subject, particularly a mammalian subject, for whom diagnosis, prognosis, or therapy is desired, for example, a human.

The phrase “safe effector immune cell” or “safe effector cell” includes those cells described by the invention that express at least one bicistronic iCAR/aCAR construct, or portion thereof, as described herein, or exhibit co-expression of monocistronic aCAR and iCAR constructs. In some embodiments, the “safe effector immune cell” or “safe effector cell” is capable of administration to a subject. In some embodiments, the “safe effector immune cell” or “safe effector cell” further expresses at least one bicistronic iCAR/aCAR construct, or portion thereof, or exhibit co-expression of monocistronic aCAR and iCAR constructs, as described herein. In some embodiments, the “safe effector immune cell” or “safe effector cell” is a CD4+ cell. In some embodiments, the “safe effector immune cell” or “safe effector cell” is a CD8+ cell.

The term “CD4+ cell” or “CD4 cell” as used herein refers to a T cell that expresses CD4 on the surface thereof. The term “CD4+ CAR T cell” as used herein refers to a T cell that expresses CD4 on the surface thereof as well as a CAR. As used herein, the term “CD4+ CAR T cell” includes those cells described by the invention that express at least one bicistronic iCAR/aCAR construct, or portion thereof, as described herein, or exhibit co-expression of monocistronic aCAR and iCAR constructs. Methods for isolating CD4+ cells, or enriching for CD4+ cells, are readily apparent to those skilled in the art. A non-limiting example is isolation of CD4+ cells from peripheral blood mononuclear cells (PBMCs) or from a transfected cell population using immunomagnetic negative selection, for example, using an EasySep™ procedure that involves labeling unwanted cells with antibody complexes and magnetic particles, and separating the magnetically labeled cells from the untouched desired cells by using an EasySep™ magnet and pouring or pipetting the desired cells into a new tube.

The term “CD8+ cell” or “CD8 cell” as used herein refers to a T cell that expresses CD8 on the surface thereof. The term “CD8+ CAR T cell” as used herein refers to a T cell that expresses CD8 on the surface thereof as well as a CAR. As used herein, the term “CD8+ CAR T cell” includes those cells described by the invention that express at least one bicistronic iCAR/aCAR construct, or portion thereof, as described herein, or exhibit co-expression of monocistronic aCAR and iCAR constructs. Methods for isolating CD8+ cells, or enriching for CD8+ cells, are readily apparent to those skilled in the art. A non-limiting example is isolation of CD8+ cells from PBMCs or from a transfected cell population using immunomagnetic negative selection, for example, using an EasySep™ procedure that involves labeling unwanted cells with antibody complexes and magnetic particles, and separating the magnetically labeled cells from the untouched desired cells by using an EasySep™ magnet and pouring or pipetting the desired cells into a new tube.

The term “untouched” or “unsorted” as used herein refers to cells that did not undergo any purification or separation step.

Pharmaceutical compositions for use in accordance with the present invention may be formulated in conventional manner using one or more physiologically acceptable carriers or excipients. The carrier(s) must be “acceptable” in the sense of being compatible with the other ingredients of the composition and not deleterious to the recipient thereof.

The phrase “effective amount” or “therapeutically effective amount” are used interchangeably herein, and refer to an amount of a compound, formulation, material, or composition, as described herein effective to achieve a particular biological result.

The term “peripheral blood mononuclear cell (PBMC)” as used herein refers to any blood cell having a round nucleus, such as a lymphocyte, or a monocyte. Methods for isolating PBMCs from blood are readily apparent to those skilled in the art. A non-limiting example is the extraction of these cells from whole blood using ficoll, a hydrophilic polysaccharide that separates layers of blood, with monocytes and lymphocytes forming a buffy coat under a layer of plasma or by leukapheresis, the preparation of leukocyte concentrates with the return of red cells and leukocyte-poor plasma to the donor.

The term “cancer” as used herein is defined as disease characterized by the rapid and uncontrolled growth of aberrant cells. Cancer cells can spread locally or through the bloodstream and lymphatic system to other parts of the body. Examples of various cancers include but are not limited to, breast cancer, prostate cancer, ovarian cancer, cervical cancer, skin cancer, pancreatic cancer, colorectal cancer, renal cancer, liver cancer, brain cancer, lymphoma, leukemia, lung cancer, glioma, and the like.

### III. Car-T System: iCARs and aCARs

LOH, being a genomic event, results in a total loss of a specific variant from the tumor with a very rare probability of gaining back the lost allele. If the LOH event occurs very early in the development of tumors, it ensures a uniform target signature in all tumor cells derived from the initial pre-malignant tissue including metastatic tumors. Additionally, LOH occurs in almost all types of cancer and this concept can therefore be relied upon as a universal tool for developing markers relevant to all these cancer types. Since the LOH events are to some extent random, the present invention further provides for selection of personalized tumor markers for each individual cancer patient, based on the specific LOH events which took place in that patient. The tools relied upon to execute this concept, the aCARs and the iCARs, are well-known and can be easily prepared using methods well-known in the art as taught for example, in WO 2015/142314 and in U.S. Pat. No. 9,745,368, both incorporated by reference as if fully disclosed herein.

According to one strategy, the two CARs in every given pair specifically recognize the product of a different allelic variant of the same target gene for which the patient is heterozygous. The basic principle is as follows: the aCAR targets an allelic variant of a selected cell surface protein that is expressed by the given tumor cells and is not affected by LOH while the iCAR targets the product encoded by the allelic variant of the same gene that has been lost from these tumor cells due to LOH. In other normal tissues of that individual patient that express the said gene, both alleles are present and are known to be equally functional, that is, expression is biallelic in all tissues (in contrast to other genes which may exhibit random monoallelic expression (Chess, 2012; Savova et al., 2016). In one scenario, the two CARs target two related epitopes residing at the same location on the protein product, which differ by one, or only few amino acids. In another scenario, the aCAR targets a non-polymorphic epitope on the same protein while the iCAR is allele-specific. In these embodiments, the density of the aCAR epitope on normal cells would generally be two-fold higher than that of the iCAR one. In some embodiments, a single nucleic acid vector encodes both the aCAR and iCAR, as exemplified with the bicistronic constructs described herein. In some embodiments, the aCAR and iCAR are encoded by separate nucleic acid vectors and co-expressed.

Care must be taken to ensure that the inhibitory signal transmitted by the iCAR is dominant over the aCAR signal and that cross-recognition between the iCAR and the aCAR is limited and/or negligible. Dominance of the iCAR guarantees that activation of the killer cell upon encounter with normal cells expressing both alleles would be prevented. This default brake would not operate upon engagement with tumor cells: in the absence of its target antigen the iCAR would not deliver inhibitory signals, thus unleashing the anticipated aCAR-mediated cellular activation and subsequent tumor cell lysis. Dominance of the iCARs over their aCARs counterparts is a significant portion of how the system functions. The present invention provides novel bicistronic iCAR/aCAR constructs that function in this manner, as well as methods for co-transduction of monocistronic aCAR and iCAR constructs.

The bicistronic constructs of the present invention comprise the following components: an iCAR and aCAR connected via a linker domain. In some embodiments, the iCAR (protective) portion comprises an iCAR scFv, a hinge transmembrane (TM) domain, and inhibitory domain. In some embodiments, the aCAR (efficacy) portion comprises an aCAR scFv, a hinge transmembrane (TM) domain, a co-stimulatory domain, and a CD3 zeta domain.

i. Bicistronic Sequences

In some embodiments, the bicistronic iCAR/aCAR comprises an amino acid sequence encoded by a nucleic acid sequence selected from the group consisting of SEQ ID NO:1, SEQ ID NO:3, SEQ ID NO:5, SEQ ID NO:7, SEQ ID NO:9, SEQ ID NO:11, SEQ ID NO:13, SEQ ID NO:15, SEQ ID NO:17, SEQ ID NO:19, SEQ ID NO:21, SEQ ID NO:23, SEQ ID NO:25, SEQ ID NO:27, SEQ ID NO:29, SEQ ID NO:31, SEQ ID NO:33, SEQ ID NO:35, SEQ ID NO:275, SEQ ID NO:277, SEQ ID NO:279, SEQ ID NO:281, SEQ ID NO:321, SEQ ID NO:323, and SEQ ID NO:325, as provided in Table 1 below. In some embodiments, the bicistronic iCAR/aCAR comprise an amino acid sequence encoded by a nucleic acid sequence comprising SEQ ID NO:1. In some embodiments, the bicistronic iCAR/aCAR comprise an amino acid sequence encoded by a nucleic acid sequence comprising SEQ ID NO:3. In some embodiments, the bicistronic iCAR/aCAR comprise an amino acid sequence encoded by a nucleic acid sequence comprising SEQ ID NO:5. In some embodiments, the bicistronic iCAR/aCAR comprise an amino acid sequence encoded by a nucleic acid sequence comprising SEQ ID NO:7. In some embodiments, the bicistronic iCAR/aCAR comprise an amino acid sequence encoded by a nucleic acid sequence comprising SEQ ID NO:9. In some embodiments, the bicistronic iCAR/aCAR comprise an amino acid sequence encoded by a nucleic acid sequence comprising SEQ ID NO:11. In some embodiments, the bicistronic iCAR/aCAR comprise an amino acid sequence encoded by a nucleic acid sequence comprising SEQ ID NO:13. In some embodiments, the bicistronic iCAR/aCAR comprise an amino acid sequence encoded by a nucleic acid sequence comprising SEQ ID NO:15. In some embodiments, the bicistronic iCAR/aCAR comprise an amino acid sequence encoded by a nucleic acid sequence comprising SEQ ID NO:17. In some embodiments, the bicistronic iCAR/aCAR comprise an amino acid sequence encoded by a nucleic acid sequence comprising SEQ ID NO:19. In some embodiments, the bicistronic iCAR/aCAR comprise an amino acid sequence encoded by a nucleic acid sequence comprising SEQ ID NO:21. In some embodiments, the bicistronic iCAR/aCAR comprise an amino acid sequence encoded by a nucleic acid sequence comprising SEQ ID NO:23. In some embodiments, the bicistronic iCAR/aCAR comprise an amino acid sequence encoded by a nucleic acid sequence comprising SEQ ID NO:25. In some embodiments, the bicistronic iCAR/aCAR comprise an amino acid sequence encoded by a nucleic acid sequence comprising SEQ ID NO:27. In some embodiments, the bicistronic iCAR/aCAR comprise an amino acid sequence encoded by a nucleic acid sequence comprising SEQ ID NO:29. In some embodiments, the bicistronic iCAR/aCAR comprise an amino acid sequence encoded by a nucleic acid sequence comprising SEQ ID NO:31. In some embodiments, the bicistronic iCAR/aCAR comprise an amino acid sequence encoded by a nucleic acid sequence comprising SEQ ID NO:33. In some embodiments, the bicistronic iCAR/aCAR comprise an amino acid sequence encoded by a nucleic acid sequence comprising SEQ ID NO:35. In some embodiments, the bicistronic iCAR/aCAR comprise an amino acid sequence encoded by a nucleic acid sequence comprising SEQ ID NO:275. In some embodiments, the bicistronic iCAR/aCAR comprise an amino acid sequence encoded by a nucleic acid sequence comprising SEQ ID NO:277. In some embodiments, the bicistronic iCAR/aCAR comprise an amino acid sequence encoded by a nucleic acid sequence comprising SEQ ID NO:279. In some embodiments, the bicistronic iCAR/aCAR comprise an amino acid sequence encoded by a nucleic acid sequence comprising SEQ ID NO:281. In some embodiments, the bicistronic iCAR/aCAR comprise an amino acid sequence encoded by a nucleic acid sequence comprising SEQ ID NO:321. In some embodiments, the bicistronic iCAR/aCAR comprise an amino acid sequence encoded by a nucleic acid sequence comprising SEQ ID NO:323. In some embodiments, the bicistronic iCAR/aCAR comprise an amino acid sequence encoded by a nucleic acid sequence comprising SEQ ID NO:325.

In some embodiments, the bicistronic iCAR/aCAR comprises an amino acid sequence selected from the group consisting of SEQ ID NO:2, SEQ ID NO:4, SEQ ID NO:6, SEQ ID NO:8, SEQ ID NO:10, SEQ ID NO:12, SEQ ID NO:14, SEQ ID NO:16, SEQ ID NO:18, SEQ ID NO:20, SEQ ID NO:22, SEQ ID NO:24, SEQ ID NO:26, SEQ ID NO:28, SEQ ID NO:30, SEQ ID NO:32, SEQ ID NO:34, SEQ ID NO:36, SEQ ID NO:276, SEQ ID NO:278, SEQ ID NO:280, SEQ ID NO:282, SEQ ID NO:322, SEQ ID NO:324, and SEQ ID NO:326 as provided in Table 1 below. In some embodiments, the bicistronic iCAR/aCAR comprises SEQ ID NO:2. In some embodiments, the bicistronic iCAR/aCAR comprises SEQ ID NO:4. In some embodiments, the bicistronic iCAR/aCAR comprises SEQ ID NO:6. In some embodiments, the bicistronic iCAR/aCAR comprises SEQ ID NO:8. In some embodiments, the bicistronic iCAR/aCAR comprises SEQ ID NO:10. In some embodiments, the bicistronic iCAR/aCAR comprises SEQ ID NO:12. In some embodiments, the bicistronic iCAR/aCAR comprises SEQ ID NO:14. In some embodiments, the bicistronic iCAR/aCAR comprises SEQ ID NO:16. In some embodiments, the bicistronic iCAR/aCAR comprises SEQ ID NO:18. In some embodiments, the bicistronic iCAR/aCAR comprises SEQ ID NO:20. In some embodiments, the bicistronic iCAR/aCAR comprises SEQ ID NO:22. In some embodiments, the bicistronic iCAR/aCAR comprises SEQ ID NO:24. In some embodiments, the bicistronic iCAR/aCAR comprises SEQ ID NO:26. In some embodiments, the bicistronic iCAR/aCAR comprises SEQ ID NO:28. In some embodiments, the bicistronic iCAR/aCAR comprises SEQ ID NO:30. In some embodiments, the bicistronic iCAR/aCAR comprises SEQ ID NO:32, SEQ ID NO:34. In some embodiments, the bicistronic iCAR/aCAR comprises SEQ ID NO:36. In some embodiments, the bicistronic iCAR/aCAR comprises SEQ ID NO:276. In some embodiments, the bicistronic iCAR/aCAR comprises SEQ ID NO:278. In some embodiments, the bicistronic iCAR/aCAR comprises SEQ ID NO:280. In some embodiments, the bicistronic iCAR/aCAR comprises SEQ ID NO:282. In some embodiments, the bicistronic iCAR/aCAR comprises SEQ ID NO:322. In some embodiments, the bicistronic iCAR/aCAR comprises SEQ ID NO:324. In some embodiments, the bicistronic iCAR/aCAR comprises SEQ ID NO:326.

TABLE 1
Bicistonic iCAR/aCARs: nucleic acid and amino acid sequences

| Sequence | SEQ ID | |
|---|---|---|
| name | NO: | Polynucleotide or polypeptide sequences |
| MC0280- | SEQ ID | ATGGCACTGCCAGTGACCGCCCTGCTGCTGCCTCTGGCC |
| BB7.2_28_ | NO: 1 | CTGCTGCTGCACGCAGCCAGACCCCAGGTGCAGCTGCA |
| PD1_HER2 | | GCAGTCTGGACCTGAGCTGGTGAAGCCAGGAGCCTCCG |
| Nucleotide | | TGAAGATGTCTTGCAAGGCCAGCGGCTACACCTTCACAT |
| sequence | | CTTATCACATCCAGTGGGTGAAGCAGCGGCCCGGACAG |
| (VR280) | | GGCCTGGAGTGGATCGGATGGATCTACCCAGGCGACGG |
| | | CTCCACACAGTATAACGAGAAGTTCAAGGGCAAGACCA |
| | | CACTGACCGCCGATAAGAGCAGCAGCACCGCCTACATG |
| | | CTGCTGAGCAGCCTGACCAGCGAGGACAGCGCCATCTA |
| | | CTTTTGCGCCAGGGAGGGCACATACTATGCTATGGACTA |
| | | TTGGGGCCAGGGCACCAGCGTGACAGTGTCTAGCGGAG |
| | | GAGGAGGCTCCGGAGGAGGAGGCTCTGGCGGCGGCGGC |
| | | AGCGACGTGCTGATGACCCAGACACCACTGAGCCTGCC |
| | | CGTGAGCCTGGGCGATCAGGTGAGCATCTCCTGTAGATC |
| | | CTCTCAGAGCATCGTGCACTCCAACGGCAATACCTACCT |
| | | GGAGTGGTATCTGCAGAAGCCAGGCCAGTCCCCCAAGC |
| | | TGCTGATCTATAAGGTGTCTAATCGGTTCAGCGGCGTGC |
| | | CTGACAGATTTTCTGGCAGCGGCTCCGGCACCGACTTCA |
| | | CCCTGAAGATCAGCCGGGTGGAGGCAGAGGATCTGGGC |
| | | GTGTACTATTGTTTCCAGGGCTCCCACGTGCCACGCACC |
| | | TTTGGCGGCGGTACCAAGCTGGAGATCAAGATTGAAGTT |
| | | ATGTATCCTCCTCCTTACCTAGACAATGAGAAGAGCAAT |
| | | GGAACCATTATCCATGTGAAAGGGAAACACCTTTGTCCA |
| | | AGTCCCCTATTTCCCGGGCCTTCGAAGCCCTTTTGGGTG |
| | | CTGGTGGTGGTTGGTGGAGTCCTGGCTTGCTATAGCTTG |
| | | CTAGTAACAGTAGCGTTTATTATTTTCTGGGTGTGCAGC |
| | | AGGGCCGCCCGCGGCACCATCGGCGCCAGGCGCACAGG |
| | | CCAGCCTCTGAAGGAGGACCCTTCCGCCGTGCCAGTGTT |
| | | CTCTGTGGACTACGGCGAGCTGGATTTTCAGTGGCGGGA |
| | | GAAAACCCCAGAGCCACCTGTGCCCTGCGTGCCTGAGC |
| | | AGACCGAGTATGCCACAATCGTGTTTCCATCCGGAATGG |
| | | GCACAAGCTCCCCTGCAAGGAGAGGCAGCGCCGACGGA |
| | | CCACGGTCCGCCCAGCCACTGCGGCCCGAGGATGGCCA |
| | | CTGTTCTTGGCCCCTGCGGAGAAAGCGTGGATCCGGGGA |
| | | AGGCCGAGGCTCCCTTCTAACATGTGGAGATGTCGAGG |
| | | AAAACCCTGGCCCTATGGCGCTGCCAGTCACTGCATTGT |
| | | TATTGCCTCTGGCCCTGCTTCTCCATGCGGCGCGCCCAG |
| | | AAGTGCAGCTGGTCGAGAGCGGAGGCGGACTGGTTCAA |
| | | CCCGGAGGCAGCTTGAGACTGTCCTGCGCGGCCAGCGG |
| | | CTTCAACATCAAGGATACCTATATCCACTGGGTGAGGCA |
| | | GGCTCCAGGAAAGGGCCTGGAGTGGGTGGCAAGGATTT |
| | | ACCCTACTAATGGATATACACGCTACGCTGATTCCGTGA |
| | | AGGGACGCTTTACAATCTCAGCAGATACATCCAAAAAC |
| | | ACGGCCTATTTACAGATGAATAGTTTGCGGGCCGAAGAC |
| | | ACGGCTGTATACTATTGTTCTCGGTGGGGGGGCGATGGA |
| | | TTTTATGCGATGGATTACTGGGGCCAGGGCACCCTGGTA |
| | | ACCGTGTCAAGCGGCTCAACATCCGGGTCCGGTAAGCC |
| | | GGGCTCCGGCGAGGGGTCTACAAAGGGAGATATACAGA |
| | | TGACACAGTCCCCCAGTTCCCTGTCCGCCTCAGTGGGAG |
| | | ACCGAGTGACGATTACCTGTCGTGCCAGCCAGGACGTCA |
| | | ATACCGCCGTCGCTTGGTATCAGCAAAAACCAGGCAAG |
| | | GCCCCGAAACTATTGATCTACAGTGCCTCTTTTCTGTACT |
| | | CCGGGGTGCCGAGCAGATTTAGTGGCTCCAGGAGCGGA |
| | | ACCGATTTCACCCTAACCATTTCCAGTTTGCAGCCAGAG |
| | | GATTTCGCGACCTATTACTGCCAGCAACACTACACCACA |
| | | CCGCCAACTTTCGGACAAGGAACCAAGGTTGAAATCAA |
| | | AACTACGACCCCAGCACCTAGACCTCCCACCCCAGCTCC |
| | | AACTATAGCTTCCCAGCCATTGTCTCTCCGGCCAGAGGC |
| | | GTGTCGACCAGCCGCTGGAGGGGCCGTTCATACAAGAG |
| | | GACTCGATTTCGCTTGCGATATCTACATATGGGCCCCTC |
| | | TTGCCGGGACATGCGGTGTCCTGCTTCTAAGCTTGGTTA |
| | | TTACCCTCTATTGCAAACGCGGCCGCAAGAAACTGCTCT |
| | | ACATCTTTAAACAGCCGTTCATGAGGCCTGTGCAGACAA |
| | | CGCAGGAAGAGGATGGCTGTAGTTGTCGGTTTCCGGAA |
| | | GAGGAAGAGGGGGGCTGCGAGTTGCGTGTCAAATTTTC |
| | | TCGGTCTGCCGACGCCCCCGCGTACCAGCAAGGGCAGA |
| | | ACCAGCTTTATAATGAGCTGAATCTTGGACGACGGGAG |
| | | GAATATGACGTGCTTGACAAGAGGCGAGGTAGGGACCC |
| | | TGAGATGGGGGGAAAACCTCGGAGGAAAAACCCACAGG |
| | | AAGGCCTGTATAACGAACTGCAGAAGGACAAGATGGCT |
| | | GAAGCCTACTCTGAGATTGGAATGAAAGGGGAACGCAG |
| | | ACGCGGCAAGGGCCATGATGGCCTCTACCAAGGTCTAA |
| | | GCACTGCCACCAAGGACACCTATGACGCACTCCACATGC |
| | | AAGCTCTACCTCCCCGTTGATAA |
| MC0280- | SEQ ID | MALPVTALLLPLALLLHAARPQVQLQQSGPELVKPGASVK |
| BB7.2_28_ | NO: 2 | MSCKASGYTFTSYHIQWVKQRPGQGLEWIGWIYPGDGSTQ |
| PD1_HER2 | | YNEKFKGKTTLTADKSSSTAYMLLSSLTSEDSAIYFCAREG |
| Protein | | TYYAMDYWGQGTSVTVSSGGGGSGGGGSGGGGSDVLMT |
| sequence | | QTPLSLPVSLGDQVSISCRSSQSIVHSNGNTYLEWYLQKPG |
| (VR280) | | QSPKLLIYKVSNRFSGVPDRFSGSGSGTDFTLKISRVEAEDL |
| | | GVYYCFQGSHVPRTFGGGTKLEIKIEVMYPPPYLDNEKSN |
| | | GTIIHVKGKHLCPSPLFPGPSKPFWVLVVVGGVLACYSLLV |
| | | TVAFIIFWVCSRAARGTIGARRTGQPLKEDPSAVPVFSVDY |
| | | GELDFQWREKTPEPPVPCVPEQTEYATIVFPSGMGTSSPAR |
| | | RGSADGPRSAQPLRPEDGHCSWPLRRKRGSGEGRGSLLTC |
| | | GDVEENPGPMALPVTALLLPLALLLHAARPEVQLVESGGG |
| | | LVQPGGSLRLSCAASGFNIKDTYIHWVRQAPGKGLEWVAR |
| | | IYPTNGYTRYADSVKGRFTISADTSKNTAYLQMNSLRAED |
| | | TAVYYCSRWGGDGFYAMDYWGQGTLVTVSSGSTSGSGK |
| | | PGSGEGSTKGDIQMTQSPSSLSASVGDRVTITCRASQDVNT |
| | | AVAWYQQKPGKAPKLLIYSASFLYSGVPSRFSGSRSGTDFT |
| | | LTISSLQPEDFATYYCQQHYTTPPTFGQGTKVEIKTTTPAPR |
| | | PPTPAPTIASQPLSLRPEACRPAAGGAVHTRGLDFACDIYIW |
| | | APLAGTCGVLLLSLVITLYCKRGRKKLLYIFKQPFMRPVQT |
| | | TQEEDGCSCRFPEEEEGGCELRVKFSRSADAPAYQQGQNQ |
| | | LYNELNLGRREEYDVLDKRRGRDPEMGGKPRRKNPQEGL |
| | | YNELQKDKMAEAYSEIGMKGERRRGKGHDGLYQGLSTAT |
| | | KDTYDALHMQALPPR |
| MC0281- | SEQ ID | ATGGCACTGCCAGTGACCGCCCTGCTGCTGCCTCTGGCC |
| BB7.2_28_ | NO: 3 | CTGCTGCTGCACGCAGCCAGACCCCAGGTGCAGCTGCA |
| PD1_EGFR | | GCAGTCTGGACCTGAGCTGGTGAAGCCAGGAGCCTCCG |
| nucleotide | | TGAAGATGTCTTGCAAGGCCAGCGGCTACACCTTCACAT |
| Sequence | | CTTATCACATCCAGTGGGTGAAGCAGCGGCCCGGACAG |
| (VR281) | | GGCCTGGAGTGGATCGGATGGATCTACCCAGGCGACGG |
| | | CTCCACACAGTATAACGAGAAGTTCAAGGGCAAGACCA |
| | | CACTGACCGCCGATAAGAGCAGCAGCACCGCCTACATG |
| | | CTGCTGAGCAGCCTGACCAGCGAGGACAGCGCCATCTA |
| | | CTTTTGCGCCAGGGAGGGCACATACTATGCTATGGACTA |
| | | TTGGGGCCAGGGCACCAGCGTGACAGTGTCTAGCGGAG |
| | | GAGGAGGCTCCGGAGGAGGAGGCTCTGGCGGCGGCGGC |
| | | AGCGACGTGCTGATGACCCAGACACCACTGAGCCTGCC |
| | | CGTGAGCCTGGGCGATCAGGTGAGCATCTCCTGTAGATC |
| | | CTCTCAGAGCATCGTGCACTCCAACGGCAATACCTACCT |
| | | GGAGTGGTATCTGCAGAAGCCAGGCCAGTCCCCCAAGC |
| | | TGCTGATCTATAAGGTGTCTAATCGGTTCAGCGGCGTGC |
| | | CTGACAGATTTTCTGGCAGCGGCTCCGGCACCGACTTCA |
| | | CCCTGAAGATCAGCCGGGTGGAGGCAGAGGATCTGGGC |
| | | GTGTACTATTGTTTCCAGGGCTCCCACGTGCCACGCACC |
| | | TTTGGCGGCGGTACCAAGCTGGAGATCAAGATTGAAGTT |
| | | ATGTATCCTCCTCCTTACCTAGACAATGAGAAGAGCAAT |
| | | GGAACCATTATCCATGTGAAAGGGAAACACCTTTGTCCA |
| | | AGTCCCCTATTTCCCGGGCCTTCGAAGCCCTTTTGGGTG |
| | | CTGGTGGTGGTTGGTGGAGTCCTGGCTTGCTATAGCTTG |
| | | CTAGTAACAGTAGCGTTTATTATTTTCTGGGTGTGCAGC |
| | | AGGGCCGCCCGCGGCACCATCGGCGCCAGGCGCACAGG |
| | | CCAGCCTCTGAAGGAGGACCCTTCCGCCGTGCCAGTGTT |
| | | CTCTGTGGACTACGGCGAGCTGGATTTTCAGTGGCGGGA |
| | | GAAAACCCCAGAGCCACCTGTGCCCTGCGTGCCTGAGC |
| | | AGACCGAGTATGCCACAATCGTGTTTCCATCCGGAATGG |
| | | GCACAAGCTCCCCTGCAAGGAGAGGCAGCGCCGACGGA |
| | | CCACGGTCCGCCCAGCCACTGCGGCCCGAGGATGGCCA |
| | | CTGTTCTTGGCCCCTGCGGAGAAAGCGTGGATCCGGGGA |
| | | AGGCCGAGGCTCCCTTCTAACATGTGGAGATGTCGAGG |
| | | AAAACCCTGGCCCTATGGCGCTGCCAGTCACTGCATTGT |
| | | TATTGCCTCTGGCCCTGCTTCTCCATGCGGCGCGCCCAC |
| | | AAGTGCAGCTGAAACAGAGCGGACCAGGACTGGTTCAA |
| | | CCCAGCCAGAGCTTGAGCATCACGTGCACGGTTAGCGG |
| | | CTTCAGTCTGACCAATTATGGTGTGCACTGGGTGAGGCA |
| | | GTCTCCAGGAAAGGGCCTGGAGTGGCTTGGAGTCATTTG |
| | | GAGCGGTGGGAATACAGATTACAATACACCTTTTACGTC |
| | | ACGTCTCTCCATTAACAAGGACAACTCCAAATCCCAAGT |
| | | ATTTTTCAAAATGAATAGCCTGCAGAGTAATGATACCGC |
| | | CATCTATTACTGTGCACGAGCTTTGACATATTACGACTA |
| | | TGAATTTGCCTATTGGGGTCAAGGCACGCTGGTGACCGT |
| | | ATCAGGCTCAACATCCGGGTCCGGTAAGCCGGGCTCCG |
| | | GCGAGGGGTCTACAAAGGGAGACATCCTTCTGACACAG |
| | | AGCCCCGTGATCCTGTCCGTGTCCCCCGGCGAGAGAGTA |
| | | TCATTTTCCTGTAGGGCTTCTCAGAGCATCGGAACAAAT |
| | | ATCCACTGGTATCAGCAACGGACTAACGGATCACCTCGC |
| | | CTGCTCATAAAGTACGCCAGTGAATCTATTAGTGGCATA |
| | | CCGAGCCGCTTCAGCGGGAGTGGCTCCGGCACAGACTTT |
| | | ACTCTGAGTATAAATTCCGTGGAATCTGAGGACATCGCG |
| | | GACTATTACTGCCAGCAAAACAATAACTGGCCCACCAC |
| | | GTTCGGCGCGGGAACTAAACTAGAACTAAAGACTACGA |
| | | CCCCAGCACCTAGACCTCCCACCCCAGCTCCAACTATAG |
| | | CTTCCCAGCCATTGTCTCTCCGGCCAGAGGCGTGTCGAC |
| | | CAGCCGCTGGAGGGGCCGTTCATACAAGAGGACTCGAT |
| | | TTCGCTTGCGATATCTACATATGGGCCCCTCTTGCCGGG |
| | | ACATGCGGTGTCCTGCTTCTAAGCTTGGTTATTACCCTCT |
| | | ATTGCAAACGCGGCCGCAAGAAACTGCTCTACATCTTTA |
| | | AACAGCCGTTCATGAGGCCTGTGCAGACAACGCAGGAA |
| | | GAGGATGGCTGTAGTTGTCGGTTTCCGGAAGAGGAAGA |
| | | GGGGGGCTGCGAGTTGCGTGTCAAATTTTCTCGGTCTGC |
| | | CGACGCCCCCGCGTACCAGCAAGGGCAGAACCAGCTTT |
| | | ATAATGAGCTGAATCTTGGACGACGGGAGGAATATGAC |
| | | GTGCTTGACAAGAGGCGAGGTAGGGACCCTGAGATGGG |
| | | GGGAAAACCTCGGAGGAAAAACCCACAGGAAGGCCTGT |
| | | ATAACGAACTGCAGAAGGACAAGATGGCTGAAGCCTAC |
| | | TCTGAGATTGGAATGAAAGGGGAACGCAGACGCGGCAA |
| | | GGGCCATGATGGCCTCTACCAAGGTCTAAGCACTGCCAC |
| | | CAAGGACACCTATGACGCACTCCACATGCAAGCTCTACC |
| | | TCCCCGTTGATAA |
| MC0281- | SEQ ID | MALPVTALLLPLALLLHAARPQVQLQQSGPELVKPGASVK |
| BB7.2_28_ | NO: 4 | MSCKASGYTFTSYHIQWVKQRPGQGLEWIGWIYPGDGSTQ |
| PD1_EGFR | | YNEKFKGKTTLTADKSSSTAYMLLSSLTSEDSAIYFCAREG |
| Protein | | TYYAMDYWGQGTSVTVSSGGGGSGGGGSGGGGSDVLMT |
| Sequence | | QTPLSLPVSLGDQVSISCRSSQSIVHSNGNTYLEWYLQKPG |
| (VR281) | | QSPKLLIYKVSNRFSGVPDRFSGSGSGTDFTLKISRVEAEDL |
| | | GVYYCFQGSHVPRTFGGGTKLEIKIEVMYPPPYLDNEKSN |
| | | GTIIHVKGKHLCPSPLFPGPSKPFWVLVVVGGVLACYSLLV |
| | | TVAFIIFWVCSRAARGTIGARRTGQPLKEDPSAVPVFSVDY |
| | | GELDFQWREKTPEPPVPCVPEQTEYATIVFPSGMGTSSPAR |
| | | RGSADGPRSAQPLRPEDGHCSWPLRRKRGSGEGRGSLLTC |
| | | GDVEENPGPMALPVTALLLPLALLLHAARPQVQLKQSGPG |
| | | LVQPSQSLSITCTVSGFSLTNYGVHWVRQSPGKGLEWLGVI |
| | | WSGGNTDYNTPFTSRLSINKDNSKSQVFFKMNSLQSNDTAI |
| | | YYCARALTYYDYEFAYWGQGTLVTVSGSTSGSGKPGSGE |
| | | GSTKGDILLTQSPVILSVSPGERVSFSCRASQSIGTNIHWYQ |
| | | QRTNGSPRLLIKYASESISGIPSRFSGSGSGTDFTLSINSVESE |
| | | DIADYYCQQNNNWPTTFGAGTKLELKTTTPAPRPPTPAPTI |
| | | ASQPLSLRPEACRPAAGGAVHTRGLDFACDIYIWAPLAGT |
| | | CGVLLLSLVITLYCKRGRKKLLYIFKQPFMRPVQTTQEEDG |
| | | CSCRFPEEEEGGCELRVKFSRSADAPAYQQGQNQLYNELN |
| | | LGRREEYDVLDKRRGRDPEMGGKPRRKNPQEGLYNELQK |
| | | DKMAEAYSEIGMKGERRRGKGHDGLYQGLSTATKDTYDA |
| | | LHMQALPPR |
| MC0282- | SEQ ID | ATGGCACTGCCAGTGACCGCCCTGCTGCTGCCTCTGGCC |
| 3PF12_28_ | NO: 5 | CTGCTGCTGCACGCAGCCAGACCCCAAGTGCAACTAGTC |
| PD1_HER2 | | CAATCAGGTGGAGGCGTCGTGCAACCTGGAGGGTCCCT |
| Nucleotide | | CCGCGTTAGCTGCGCCGCATCAGGCGTTACCTTGTCAGA |
| Sequence | | CTACGGCATGCATTGGGTTAGGCAAGCCCCCGGCAAGG |
| (VR282) | | GGCTCGAATGGATGGCTTTCATTCGGAATGACGGGAGC |
| | | GATAAATATTACGCGGATTCAGTTAAAGGGCGGTTCACC |
| | | ATCAGCCGCGACAATAGCAAAAAGACGGTCTCCTTACA |
| | | GATGTCCAGCTTGCGGGCCGAAGACACGGCTGTATACTA |
| | | TTGTGCTAAAAATGGCGAGAGCGGCCCCCTGGATTACTG |
| | | GTACTTTGACCTGTGGGGCAGAGGCACCCTGGTCACGGT |
| | | GTCCTCTGGAGGAGGAGGCTCCGGAGGAGGAGGCTCTG |
| | | GCGGCGGCGGCAGCGACATTGTAATGACCCAGTCACCC |
| | | TCCTTCCTTAGTGCCTCAGTCGGAGACCGCGTGACTATC |
| | | ACTTGTCGTGCCTCACACGGAATTAATAACTACCTCGCT |
| | | TGGTATCAGCAAAAACCAGGCAAGGCCCCGAAACTATT |
| | | GATCTACGCCGCATCTACTCTGCAGAGCGGAGTACCGAG |
| | | CAGATTTAGTGGTTCCGGCAGCGGAACCGAGTTCACCCT |
| | | AACCATTTCCAGTTTGCAGCCAGAGGATTTCGCGACCTA |
| | | TTACTGCCAGCAATACGATTCATACCCGCCAACTTTCGG |
| | | AAGAGGTACCAAGGTTGAAATCAAGATTGAAGTTATGT |
| | | ATCCTCCTCCTTACCTAGACAATGAGAAGAGCAATGGAA |
| | | CCATTATCCATGTGAAAGGGAAACACCTTTGTCCAAGTC |
| | | CCCTATTTCCCGGGCCTTCGAAGCCCTTTTGGGTGCTGGT |
| | | GGTGGTTGGTGGAGTCCTGGCTTGCTATAGCTTGCTAGT |
| | | AACAGTAGCGTTTATTATTTTCTGGGTGTGCAGCAGGGC |
| | | CGCCCGCGGCACCATCGGCGCCAGGCGCACAGGCCAGC |
| | | CTCTGAAGGAGGACCCTTCCGCCGTGCCAGTGTTCTCTG |
| | | TGGACTACGGCGAGCTGGATTTTCAGTGGCGGGAGAAA |
| | | ACCCCAGAGCCACCTGTGCCCTGCGTGCCTGAGCAGACC |
| | | GAGTATGCCACAATCGTGTTTCCATCCGGAATGGGCACA |
| | | AGCTCCCCTGCAAGGAGAGGCAGCGCCGACGGACCACG |
| | | GTCCGCCCAGCCACTGCGGCCCGAGGATGGCCACTGTTC |
| | | TTGGCCCCTGCGGAGAAAGCGTGGATCCGGGGAAGGCC |
| | | GAGGCTCCCTTCTAACATGTGGAGATGTCGAGGAAAAC |
| | | CCTGGCCCTATGGCGCTGCCAGTCACTGCATTGTTATTG |
| | | CCTCTGGCCCTGCTTCTCCATGCGGCGCGCCCAGAAGTG |
| | | CAGCTGGTCGAGAGCGGAGGCGGACTGGTTCAACCCGG |
| | | AGGCAGCTTGAGACTGTCCTGCGCGGCCAGCGGCTTCAA |
| | | CATCAAGGATACCTATATCCACTGGGTGAGGCAGGCTCC |
| | | AGGAAAGGGCCTGGAGTGGGTGGCAAGGATTTACCCTA |
| | | CTAATGGATATACACGCTACGCTGATTCCGTGAAGGGAC |
| | | GCTTTACAATCTCAGCAGATACATCCAAAAACACGGCCT |
| | | ATTTACAGATGAATAGTTTGCGGGCCGAAGACACGGCT |
| | | GTATACTATTGTTCTCGGTGGGGGGGCGATGGATTTTAT |
| | | GCGATGGATTACTGGGGCCAGGGCACCCTGGTAACCGT |
| | | GTCAAGCGGCTCAACATCCGGGTCCGGTAAGCCGGGCT |
| | | CCGGCGAGGGGTCTACAAAGGGAGATATACAGATGACA |
| | | CAGTCCCCCAGTTCCCTGTCCGCCTCAGTGGGAGACCGA |
| | | GTGACGATTACCTGTCGTGCCAGCCAGGACGTCAATACC |
| | | GCCGTCGCTTGGTATCAGCAAAAACCAGGCAAGGCCCC |
| | | GAAACTATTGATCTACAGTGCCTCTTTTCTGTACTCCGG |
| | | GGTGCCGAGCAGATTTAGTGGCTCCAGGAGCGGAACCG |
| | | ATTTCACCCTAACCATTTCCAGTTTGCAGCCAGAGGATT |
| | | TCGCGACCTATTACTGCCAGCAACACTACACCACACCGC |
| | | CAACTTTCGGACAAGGAACCAAGGTTGAAATCAAAACT |
| | | ACGACCCCAGCACCTAGACCTCCCACCCCAGCTCCAACT |
| | | ATAGCTTCCCAGCCATTGTCTCTCCGGCCAGAGGCGTGT |
| | | CGACCAGCCGCTGGAGGGGCCGTTCATACAAGAGGACT |
| | | CGATTTCGCTTGCGATATCTACATATGGGCCCCTCTTGCC |
| | | GGGACATGCGGTGTCCTGCTTCTAAGCTTGGTTATTACC |
| | | CTCTATTGCAAACGCGGCCGCAAGAAACTGCTCTACATC |
| | | TTTAAACAGCCGTTCATGAGGCCTGTGCAGACAACGCAG |
| | | GAAGAGGATGGCTGTAGTTGTCGGTTTCCGGAAGAGGA |
| | | AGAGGGGGGCTGCGAGTTGCGTGTCAAATTTTCTCGGTC |
| | | TGCCGACGCCCCCGCGTACCAGCAAGGGCAGAACCAGC |
| | | TTTATAATGAGCTGAATCTTGGACGACGGGAGGAATATG |
| | | ACGTGCTTGACAAGAGGCGAGGTAGGGACCCTGAGATG |
| | | GGGGGAAAACCTCGGAGGAAAAACCCACAGGAAGGCCT |
| | | GTATAACGAACTGCAGAAGGACAAGATGGCTGAAGCCT |
| | | ACTCTGAGATTGGAATGAAAGGGGAACGCAGACGCGGC |
| | | AAGGGCCATGATGGCCTCTACCAAGGTCTAAGCACTGCC |
| | | ACCAAGGACACCTATGACGCACTCCACATGCAAGCTCTA |
| | | CCTCCCCGTTGATAA |
| MC0282- | SEQ ID | MALPVTALLLPLALLLHAARPQVQLVQSGGGVVQPGGSLR |
| 3PF12_28_ | NO: 6 | VSCAASGVTLSDYGMHWVRQAPGKGLEWMAFIRNDGSD |
| PD1_HER2 | | KYYADSVKGRFTISRDNSKKTVSLQMSSLRAEDTAVYYCA |
| Protein | | KNGESGPLDYWYFDLWGRGTLVTVSSGGGGSGGGGSGGG |
| Sequence | | GSDIVMTQSPSFLSASVGDRVTITCRASHGINNYLAWYQQ |
| (VR282) | | KPGKAPKLLIYAASTLQSGVPSRFSGSGSGTEFTLTISSLQPE |
| | | DFATYYCQQYDSYPPTFGRGTKVEIKIEVMYPPPYLDNEKS |
| | | NGTIIHVKGKHLCPSPLFPGPSKPFWVLVVVGGVLACYSLL |
| | | VTVAFIIFWVCSRAARGTIGARRTGQPLKEDPSAVPVFSVD |
| | | YGELDFQWREKTPEPPVPCVPEQTEYATIVFPSGMGTSSPA |
| | | RRGSADGPRSAQPLRPEDGHCSWPLRRKRGSGEGRGSLLT |
| | | CGDVEENPGPMALPVTALLLPLALLLHAARPEVQLVESGG |
| | | GLVQPGGSLRLSCAASGFNIKDTYIHWVRQAPGKGLEWVA |
| | | RIYPTNGYTRYADSVKGRFTISADTSKNTAYLQMNSLRAE |
| | | DTAVYYCSRWGGDGFYAMDYWGQGTLVTVSSGSTSGSG |
| | | KPGSGEGSTKGDIQMTQSPSSLSASVGDRVTITCRASQDVN |
| | | TAVAWYQQKPGKAPKLLIYSASFLYSGVPSRFSGSRSGTDF |
| | | TLTISSLQPEDFATYYCQQHYTTPPTFGQGTKVEIKTTTPAP |
| | | RPPTPAPTIASQPLSLRPEACRPAAGGAVHTRGLDFACDIYI |
| | | WAPLAGTCGVLLLSLVITLYCKRGRKKLLYIFKQPFMRPV |
| | | QTTQEEDGCSCRFPEEEEGGCELRVKFSRSADAPAYQQGQ |
| | | NQLYNELNLGRREEYDVLDKRRGRDPEMGGKPRRKNPQE |
| | | GLYNELQKDKMAEAYSEIGMKGERRRGKGHDGLYQGLST |
| | | ATKDTYDALHMQALPPR |
| MC0283- | SEQ ID | ATGGCACTGCCAGTGACCGCCCTGCTGCTGCCTCTGGCC |
| 3PF12_28_ | NO: 7 | CTGCTGCTGCACGCAGCCAGACCCCAAGTGCAACTAGTC |
| PD1_EGFR | | CAATCAGGTGGAGGCGTCGTGCAACCTGGAGGGTCCCT |
| Nucleotide | | CCGCGTTAGCTGCGCCGCATCAGGCGTTACCTTGTCAGA |
| Sequence | | CTACGGCATGCATTGGGTTAGGCAAGCCCCCGGCAAGG |
| (VR283) | | GGCTCGAATGGATGGCTTTCATTCGGAATGACGGGAGC |
| | | GATAAATATTACGCGGATTCAGTTAAAGGGCGGTTCACC |
| | | ATCAGCCGCGACAATAGCAAAAAGACGGTCTCCTTACA |
| | | GATGTCCAGCTTGCGGGCCGAAGACACGGCTGTATACTA |
| | | TTGTGCTAAAAATGGCGAGAGCGGCCCCCTGGATTACTG |
| | | GTACTTTGACCTGTGGGGCAGAGGCACCCTGGTCACGGT |
| | | GTCCTCTGGAGGAGGAGGCTCCGGAGGAGGAGGCTCTG |
| | | GCGGCGGCGGCAGCGACATTGTAATGACCCAGTCACCC |
| | | TCCTTCCTTAGTGCCTCAGTCGGAGACCGCGTGACTATC |
| | | ACTTGTCGTGCCTCACACGGAATTAATAACTACCTCGCT |
| | | TGGTATCAGCAAAAACCAGGCAAGGCCCCGAAACTATT |
| | | GATCTACGCCGCATCTACTCTGCAGAGCGGAGTACCGAG |
| | | CAGATTTAGTGGTTCCGGCAGCGGAACCGAGTTCACCCT |
| | | AACCATTTCCAGTTTGCAGCCAGAGGATTTCGCGACCTA |
| | | TTACTGCCAGCAATACGATTCATACCCGCCAACTTTCGG |
| | | AAGAGGTACCAAGGTTGAAATCAAGATTGAAGTTATGT |
| | | ATCCTCCTCCTTACCTAGACAATGAGAAGAGCAATGGAA |
| | | CCATTATCCATGTGAAAGGGAAACACCTTTGTCCAAGTC |
| | | CCCTATTTCCCGGGCCTTCGAAGCCCTTTTGGGTGCTGGT |
| | | GGTGGTTGGTGGAGTCCTGGCTTGCTATAGCTTGCTAGT |
| | | AACAGTAGCGTTTATTATTTTCTGGGTGTGCAGCAGGGC |
| | | CGCCCGCGGCACCATCGGCGCCAGGCGCACAGGCCAGC |
| | | CTCTGAAGGAGGACCCTTCCGCCGTGCCAGTGTTCTCTG |
| | | TGGACTACGGCGAGCTGGATTTTCAGTGGCGGGAGAAA |
| | | ACCCCAGAGCCACCTGTGCCCTGCGTGCCTGAGCAGACC |
| | | GAGTATGCCACAATCGTGTTTCCATCCGGAATGGGCACA |
| | | AGCTCCCCTGCAAGGAGAGGCAGCGCCGACGGACCACG |
| | | GTCCGCCCAGCCACTGCGGCCCGAGGATGGCCACTGTTC |
| | | TTGGCCCCTGCGGAGAAAGCGTGGATCCGGGGAAGGCC |
| | | GAGGCTCCCTTCTAACATGTGGAGATGTCGAGGAAAAC |
| | | CCTGGCCCTATGGCGCTGCCAGTCACTGCATTGTTATTG |
| | | CCTCTGGCCCTGCTTCTCCATGCGGCGCGCCCACAAGTG |
| | | CAGCTGAAACAGAGCGGACCAGGACTGGTTCAACCCAG |
| | | CCAGAGCTTGAGCATCACGTGCACGGTTAGCGGCTTCAG |
| | | TCTGACCAATTATGGTGTGCACTGGGTGAGGCAGTCTCC |
| | | AGGAAAGGGCCTGGAGTGGCTTGGAGTCATTTGGAGCG |
| | | GTGGGAATACAGATTACAATACACCTTTTACGTCACGTC |
| | | TCTCCATTAACAAGGACAACTCCAAATCCCAAGTATTTT |
| | | TCAAAATGAATAGCCTGCAGAGTAATGATACCGCCATCT |
| | | ATTACTGTGCACGAGCTTTGACATATTACGACTATGAAT |
| | | TTGCCTATTGGGGTCAAGGCACGCTGGTGACCGTATCAG |
| | | GCTCAACATCCGGGTCCGGTAAGCCGGGCTCCGGCGAG |
| | | GGGTCTACAAAGGGAGACATCCTTCTGACACAGAGCCC |
| | | CGTGATCCTGTCCGTGTCCCCCGGCGAGAGAGTATCATT |
| | | TTCCTGTAGGGCTTCTCAGAGCATCGGAACAAATATCCA |
| | | CTGGTATCAGCAACGGACTAACGGATCACCTCGCCTGCT |
| | | CATAAAGTACGCCAGTGAATCTATTAGTGGCATACCGAG |
| | | CCGCTTCAGCGGGAGTGGCTCCGGCACAGACTTTACTCT |
| | | GAGTATAAATTCCGTGGAATCTGAGGACATCGCGGACT |
| | | ATTACTGCCAGCAAAACAATAACTGGCCCACCACGTTCG |
| | | GCGCGGGAACTAAACTAGAACTAAAGACTACGACCCCA |
| | | GCACCTAGACCTCCCACCCCAGCTCCAACTATAGCTTCC |
| | | CAGCCATTGTCTCTCCGGCCAGAGGCGTGTCGACCAGCC |
| | | GCTGGAGGGGCCGTTCATACAAGAGGACTCGATTTCGCT |
| | | TGCGATATCTACATATGGGCCCCTCTTGCCGGGACATGC |
| | | GGTGTCCTGCTTCTAAGCTTGGTTATTACCCTCTATTGCA |
| | | AACGCGGCCGCAAGAAACTGCTCTACATCTTTAAACAGC |
| | | CGTTCATGAGGCCTGTGCAGACAACGCAGGAAGAGGAT |
| | | GGCTGTAGTTGTCGGTTTCCGGAAGAGGAAGAGGGGGG |
| | | CTGCGAGTTGCGTGTCAAATTTTCTCGGTCTGCCGACGC |
| | | CCCCGCGTACCAGCAAGGGCAGAACCAGCTTTATAATG |
| | | AGCTGAATCTTGGACGACGGGAGGAATATGACGTGCTT |
| | | GACAAGAGGCGAGGTAGGGACCCTGAGATGGGGGGAA |
| | | AACCTCGGAGGAAAAACCCACAGGAAGGCCTGTATAAC |
| | | GAACTGCAGAAGGACAAGATGGCTGAAGCCTACTCTGA |
| | | GATTGGAATGAAAGGGGAACGCAGACGCGGCAAGGGCC |
| | | ATGATGGCCTCTACCAAGGTCTAAGCACTGCCACCAAGG |
| | | ACACCTATGACGCACTCCACATGCAAGCTCTACCTCCCC |
| | | GTTGATAA |
| MC0283- | SEQ ID | MALPVTALLLPLALLLHAARPQVQLVQSGGGVVQPGGSLR |
| 3PF12_28_ | NO: 8 | VSCAASGVTLSDYGMHWVRQAPGKGLEWMAFIRNDGSD |
| PD1_EGFR | | KYYADSVKGRFTISRDNSKKTVSLQMSSLRAEDTAVYYCA |
| Protein | | KNGESGPLDYWYFDLWGRGTLVTVSSGGGGSGGGGSGGG |
| Sequence | | GSDIVMTQSPSFLSASVGDRVTITCRASHGINNYLAWYQQ |
| (VR283) | | KPGKAPKLLIYAASTLQSGVPSRFSGSGSGTEFTLTISSLQPE |
| | | DFATYYCQQYDSYPPTFGRGTKVEIKIEVMYPPPYLDNEKS |
| | | NGTIIHVKGKHLCPSPLFPGPSKPFWVLVVVGGVLACYSLL |
| | | VTVAFIIFWVCSRAARGTIGARRTGQPLKEDPSAVPVFSVD |
| | | YGELDFQWREKTPEPPVPCVPEQTEYATIVFPSGMGTSSPA |
| | | RRGSADGPRSAQPLRPEDGHCSWPLRRKRGSGEGRGSLLT |
| | | CGDVEENPGPMALPVTALLLPLALLLHAARPQVQLKQSGP |
| | | GLVQPSQSLSITCTVSGFSLTNYGVHWVRQSPGKGLEWLG |
| | | VIWSGGNTDYNTPFTSRLSINKDNSKSQVFFKMNSLQSNDT |
| | | AIYYCARALTYYDYEFAYWGQGTLVTVSGSTSGSGKPGSG |
| | | EGSTKGDILLTQSPVILSVSPGERVSFSCRASQSIGTNIHWY |
| | | QQRTNGSPRLLIKYASESISGIPSRFSGSGSGTDFTLSINSVES |
| | | EDIADYYCQQNNNWPTTFGAGTKLELKTTTPAPRPPTPAPT |
| | | IASQPLSLRPEACRPAAGGAVHTRGLDFACDIYIWAPLAGT |
| | | CGVLLLSLVITLYCKRGRKKLLYIFKQPFMRPVQTTQEEDG |
| | | CSCRFPEEEEGGCELRVKFSRSADAPAYQQGQNQLYNELN |
| | | LGRREEYDVLDKRRGRDPEMGGKPRRKNPQEGLYNELQK |
| | | DKMAEAYSEIGMKGERRRGKGHDGLYQGLSTATKDTYDA |
| | | LHMQALPPR |
| MC0284- | SEQ ID | ATGGCACTGCCAGTGACCGCCCTGCTGCTGCCTCTGGCC |
| BB7.2_8_P | NO: 9 | CTGCTGCTGCACGCAGCCAGACCCCAGGTGCAGCTGCA |
| D1_HER2 | | GCAGTCTGGACCTGAGCTGGTGAAGCCAGGAGCCTCCG |
| Nucleotide | | TGAAGATGTCTTGCAAGGCCAGCGGCTACACCTTCACAT |
| Sequence | | CTTATCACATCCAGTGGGTGAAGCAGCGGCCCGGACAG |
| (VR284) | | GGCCTGGAGTGGATCGGATGGATCTACCCAGGCGACGG |
| | | CTCCACACAGTATAACGAGAAGTTCAAGGGCAAGACCA |
| | | CACTGACCGCCGATAAGAGCAGCAGCACCGCCTACATG |
| | | CTGCTGAGCAGCCTGACCAGCGAGGACAGCGCCATCTA |
| | | CTTTTGCGCCAGGGAGGGCACATACTATGCTATGGACTA |
| | | TTGGGGCCAGGGCACCAGCGTGACAGTGTCTAGCGGAG |
| | | GAGGAGGCTCCGGAGGAGGAGGCTCTGGCGGCGGCGGC |
| | | AGCGACGTGCTGATGACCCAGACACCACTGAGCCTGCC |
| | | CGTGAGCCTGGGCGATCAGGTGAGCATCTCCTGTAGATC |
| | | CTCTCAGAGCATCGTGCACTCCAACGGCAATACCTACCT |
| | | GGAGTGGTATCTGCAGAAGCCAGGCCAGTCCCCCAAGC |
| | | TGCTGATCTATAAGGTGTCTAATCGGTTCAGCGGCGTGC |
| | | CTGACAGATTTTCTGGCAGCGGCTCCGGCACCGACTTCA |
| | | CCCTGAAGATCAGCCGGGTGGAGGCAGAGGATCTGGGC |
| | | GTGTACTATTGTTTCCAGGGCTCCCACGTGCCACGCACC |
| | | TTTGGCGGCGGTACCAAGCTGGAGATCAAGACTACGAC |
| | | CCCAGCACCTAGACCTCCCACCCCAGCTCCAACTATAGC |
| | | TTCCCAGCCATTGTCTCTCCGGCCAGAGGCGTGTCGACC |
| | | AGCCGCTGGAGGGGCCGTTCATACAAGAGGACTCGATT |
| | | TCGCTTGCGATATCTACATATGGGCCCCTCTTGCCGGGA |
| | | CATGCGGTGTCCTGCTTCTAAGCTTGGTTATTACCCTCTA |
| | | TTGCTGCAGCAGGGCCGCCCGCGGCACCATCGGCGCCA |
| | | GGCGCACAGGCCAGCCTCTGAAGGAGGACCCTTCCGCC |
| | | GTGCCAGTGTTCTCTGTGGACTACGGCGAGCTGGATTTT |
| | | CAGTGGCGGGAGAAAACCCCAGAGCCACCTGTGCCCTG |
| | | CGTGCCTGAGCAGACCGAGTATGCCACAATCGTGTTTCC |
| | | ATCCGGAATGGGCACAAGCTCCCCTGCAAGGAGAGGCA |
| | | GCGCCGACGGACCACGGTCCGCCCAGCCACTGCGGCCC |
| | | GAGGATGGCCACTGTTCTTGGCCCCTGCGGAGAAAGCGT |
| | | GGATCCGGGGAAGGCCGAGGCTCCCTTCTAACATGTGG |
| | | AGATGTCGAGGAAAACCCTGGCCCTATGGCGCTGCCAG |
| | | TCACTGCATTGTTATTGCCTCTGGCCCTGCTTCTCCATGC |
| | | GGCGCGCCCAGAAGTGCAGCTGGTCGAGAGCGGAGGCG |
| | | GACTGGTTCAACCCGGAGGCAGCTTGAGACTGTCCTGCG |
| | | CGGCCAGCGGCTTCAACATCAAGGATACCTATATCCACT |
| | | GGGTGAGGCAGGCTCCAGGAAAGGGCCTGGAGTGGGTG |
| | | GCAAGGATTTACCCTACTAATGGATATACACGCTACGCT |
| | | GATTCCGTGAAGGGACGCTTTACAATCTCAGCAGATACA |
| | | TCCAAAAACACGGCCTATTTACAGATGAATAGTTTGCGG |
| | | GCCGAAGACACGGCTGTATACTATTGTTCTCGGTGGGGG |
| | | GGCGATGGATTTTATGCGATGGATTACTGGGGCCAGGGC |
| | | ACCCTGGTAACCGTGTCAAGCGGCTCAACATCCGGGTCC |
| | | GGTAAGCCGGGCTCCGGCGAGGGGTCTACAAAGGGAGA |
| | | TATACAGATGACACAGTCCCCCAGTTCCCTGTCCGCCTC |
| | | AGTGGGAGACCGAGTGACGATTACCTGTCGTGCCAGCC |
| | | AGGACGTCAATACCGCCGTCGCTTGGTATCAGCAAAAA |
| | | CCAGGCAAGGCCCCGAAACTATTGATCTACAGTGCCTCT |
| | | TTTCTGTACTCCGGGGTGCCGAGCAGATTTAGTGGCTCC |
| | | AGGAGCGGAACCGATTTCACCCTAACCATTTCCAGTTTG |
| | | CAGCCAGAGGATTTCGCGACCTATTACTGCCAGCAACAC |
| | | TACACCACACCGCCAACTTTCGGACAAGGAACCAAGGT |
| | | TGAAATCAAAATTGAAGTTATGTATCCTCCTCCTTACCT |
| | | AGACAATGAGAAGAGCAATGGAACCATTATCCATGTGA |
| | | AAGGGAAACACCTTTGTCCAAGTCCCCTATTTCCCGGGC |
| | | CTTCGAAGCCCTTTTGGGTGCTGGTGGTGGTTGGTGGAG |
| | | TCCTGGCTTGCTATAGCTTGCTAGTAACAGTAGCGTTTA |
| | | TTATTTTCTGGGTGAAACGCGGCCGCAAGAAACTGCTCT |
| | | ACATCTTTAAACAGCCGTTCATGAGGCCTGTGCAGACAA |
| | | CGCAGGAAGAGGATGGCTGTAGTTGTCGGTTTCCGGAA |
| | | GAGGAAGAGGGGGGCTGCGAGTTGCGTGTCAAATTTTC |
| | | TCGGTCTGCCGACGCCCCCGCGTACCAGCAAGGGCAGA |
| | | ACCAGCTTTATAATGAGCTGAATCTTGGACGACGGGAG |
| | | GAATATGACGTGCTTGACAAGAGGCGAGGTAGGGACCC |
| | | TGAGATGGGGGGAAAACCTCGGAGGAAAAACCCACAGG |
| | | AAGGCCTGTATAACGAACTGCAGAAGGACAAGATGGCT |
| | | GAAGCCTACTCTGAGATTGGAATGAAAGGGGAACGCAG |
| | | ACGCGGCAAGGGCCATGATGGCCTCTACCAAGGTCTAA |
| | | GCACTGCCACCAAGGACACCTATGACGCACTCCACATGC |
| | | AAGCTCTACCTCCCCGTTGATAA |
| MC0284- | SEQ ID | MALPVTALLLPLALLLHAARPQVQLQQSGPELVKPGASVK |
| BB7.2_8_P | NO: 10 | MSCKASGYTFTSYHIQWVKQRPGQGLEWIGWIYPGDGSTQ |
| D1_HER2 | | YNEKFKGKTTLTADKSSSTAYMLLSSLTSEDSAIYFCAREG |
| Protein | | TYYAMDYWGQGTSVTVSSGGGGSGGGGSGGGGSDVLMT |
| Sequence | | QTPLSLPVSLGDQVSISCRSSQSIVHSNGNTYLEWYLQKPG |
| (VR284) | | QSPKLLIYKVSNRFSGVPDRFSGSGSGTDFTLKISRVEAEDL |
| | | GVYYCFQGSHVPRTFGGGTKLEIKTTTPAPRPPTPAPTIASQ |
| | | PLSLRPEACRPAAGGAVHTRGLDFACDIYIWAPLAGTCGV |
| | | LLLSLVITLYCCSRAARGTIGARRTGQPLKEDPSAVPVFSV |
| | | DYGELDFQWREKTPEPPVPCVPEQTEYATIVFPSGMGTSSP |
| | | ARRGSADGPRSAQPLRPEDGHCSWPLRRKRGSGEGRGSLL |
| | | TCGDVEENPGPMALPVTALLLPLALLLHAARPEVQLVESG |
| | | GGLVQPGGSLRLSCAASGFNIKDTYIHWVRQAPGKGLEWV |
| | | ARIYPTNGYTRYADSVKGRFTISADTSKNTAYLQMNSLRA |
| | | EDTAVYYCSRWGGDGFYAMDYWGQGTLVTVSSGSTSGS |
| | | GKPGSGEGSTKGDIQMTQSPSSLSASVGDRVTITCRASQDV |
| | | NTAVAWYQQKPGKAPKLLIYSASFLYSGVPSRFSGSRSGT |
| | | DFTLTISSLQPEDFATYYCQQHYTTPPTFGQGTKVEIKIEVM |
| | | YPPPYLDNEKSNGTIIHVKGKHLCPSPLFPGPSKPFWVLVV |
| | | VGGVLACYSLLVTVAFIIFWVKRGRKKLLYIFKQPFMRPV |
| | | QTTQEEDGCSCRFPEEEEGGCELRVKFSRSADAPAYQQGQ |
| | | NQLYNELNLGRREEYDVLDKRRGRDPEMGGKPRRKNPQE |
| | | GLYNELQKDKMAEAYSEIGMKGERRRGKGHDGLYQGLST |
| | | ATKDTYDALHMQALPPR |
| MC0285- | SEQ ID | ATGGCACTGCCAGTGACCGCCCTGCTGCTGCCTCTGGCC |
| 3PF12_8_P | NO: 11 | CTGCTGCTGCACGCAGCCAGACCCCAAGTGCAACTAGTC |
| D1_HER2 | | CAATCAGGTGGAGGCGTCGTGCAACCTGGAGGGTCCCT |
| nucleotide | | CCGCGTTAGCTGCGCCGCATCAGGCGTTACCTTGTCAGA |
| Sequence | | CTACGGCATGCATTGGGTTAGGCAAGCCCCCGGCAAGG |
| (VR285) | | GGCTCGAATGGATGGCTTTCATTCGGAATGACGGGAGC |
| | | GATAAATATTACGCGGATTCAGTTAAAGGGCGGTTCACC |
| | | ATCAGCCGCGACAATAGCAAAAAGACGGTCTCCTTACA |
| | | GATGTCCAGCTTGCGGGCCGAAGACACGGCTGTATACTA |
| | | TTGTGCTAAAAATGGCGAGAGCGGCCCCCTGGATTACTG |
| | | GTACTTTGACCTGTGGGGCAGAGGCACCCTGGTCACGGT |
| | | GTCCTCTGGAGGAGGAGGCTCCGGAGGAGGAGGCTCTG |
| | | GCGGCGGCGGCAGCGACATTGTAATGACCCAGTCACCC |
| | | TCCTTCCTTAGTGCCTCAGTCGGAGACCGCGTGACTATC |
| | | ACTTGTCGTGCCTCACACGGAATTAATAACTACCTCGCT |
| | | TGGTATCAGCAAAAACCAGGCAAGGCCCCGAAACTATT |
| | | GATCTACGCCGCATCTACTCTGCAGAGCGGAGTACCGAG |
| | | CAGATTTAGTGGTTCCGGCAGCGGAACCGAGTTCACCCT |
| | | AACCATTTCCAGTTTGCAGCCAGAGGATTTCGCGACCTA |
| | | TTACTGCCAGCAATACGATTCATACCCGCCAACTTTCGG |
| | | AAGAGGTACCAAGGTTGAAATCAAGACTACGACCCCAG |
| | | CACCTAGACCTCCCACCCCAGCTCCAACTATAGCTTCCC |
| | | AGCCATTGTCTCTCCGGCCAGAGGCGTGTCGACCAGCCG |
| | | CTGGAGGGGCCGTTCATACAAGAGGACTCGATTTCGCTT |
| | | GCGATATCTACATATGGGCCCCTCTTGCCGGGACATGCG |
| | | GTGTCCTGCTTCTAAGCTTGGTTATTACCCTCTATTGCTG |
| | | CAGCAGGGCCGCCCGCGGCACCATCGGCGCCAGGCGCA |
| | | CAGGCCAGCCTCTGAAGGAGGACCCTTCCGCCGTGCCA |
| | | GTGTTCTCTGTGGACTACGGCGAGCTGGATTTTCAGTGG |
| | | CGGGAGAAAACCCCAGAGCCACCTGTGCCCTGCGTGCC |
| | | TGAGCAGACCGAGTATGCCACAATCGTGTTTCCATCCGG |
| | | AATGGGCACAAGCTCCCCTGCAAGGAGAGGCAGCGCCG |
| | | ACGGACCACGGTCCGCCCAGCCACTGCGGCCCGAGGAT |
| | | GGCCACTGTTCTTGGCCCCTGCGGAGAAAGCGTGGATCC |
| | | GGGGAAGGCCGAGGCTCCCTTCTAACATGTGGAGATGT |
| | | CGAGGAAAACCCTGGCCCTATGGCGCTGCCAGTCACTGC |
| | | ATTGTTATTGCCTCTGGCCCTGCTTCTCCATGCGGCGCGC |
| | | CCAGAAGTGCAGCTGGTCGAGAGCGGAGGCGGACTGGT |
| | | TCAACCCGGAGGCAGCTTGAGACTGTCCTGCGCGGCCA |
| | | GCGGCTTCAACATCAAGGATACCTATATCCACTGGGTGA |
| | | GGCAGGCTCCAGGAAAGGGCCTGGAGTGGGTGGCAAGG |
| | | ATTTACCCTACTAATGGATATACACGCTACGCTGATTCC |
| | | GTGAAGGGACGCTTTACAATCTCAGCAGATACATCCAA |
| | | AAACACGGCCTATTTACAGATGAATAGTTTGCGGGCCGA |
| | | AGACACGGCTGTATACTATTGTTCTCGGTGGGGGGGCGA |
| | | TGGATTTTATGCGATGGATTACTGGGGCCAGGGCACCCT |
| | | GGTAACCGTGTCAAGCGGCTCAACATCCGGGTCCGGTA |
| | | AGCCGGGCTCCGGCGAGGGGTCTACAAAGGGAGATATA |
| | | CAGATGACACAGTCCCCCAGTTCCCTGTCCGCCTCAGTG |
| | | GGAGACCGAGTGACGATTACCTGTCGTGCCAGCCAGGA |
| | | CGTCAATACCGCCGTCGCTTGGTATCAGCAAAAACCAGG |
| | | CAAGGCCCCGAAACTATTGATCTACAGTGCCTCTTTTCT |
| | | GTACTCCGGGGTGCCGAGCAGATTTAGTGGCTCCAGGA |
| | | GCGGAACCGATTTCACCCTAACCATTTCCAGTTTGCAGC |
| | | CAGAGGATTTCGCGACCTATTACTGCCAGCAACACTACA |
| | | CCACACCGCCAACTTTCGGACAAGGAACCAAGGTTGAA |
| | | ATCAAAATTGAAGTTATGTATCCTCCTCCTTACCTAGAC |
| | | AATGAGAAGAGCAATGGAACCATTATCCATGTGAAAGG |
| | | GAAACACCTTTGTCCAAGTCCCCTATTTCCCGGGCCTTC |
| | | GAAGCCCTTTTGGGTGCTGGTGGTGGTTGGTGGAGTCCT |
| | | GGCTTGCTATAGCTTGCTAGTAACAGTAGCGTTTATTAT |
| | | TTTCTGGGTGAAACGCGGCCGCAAGAAACTGCTCTACAT |
| | | CTTTAAACAGCCGTTCATGAGGCCTGTGCAGACAACGCA |
| | | GGAAGAGGATGGCTGTAGTTGTCGGTTTCCGGAAGAGG |
| | | AAGAGGGGGGCTGCGAGTTGCGTGTCAAATTTTCTCGGT |
| | | CTGCCGACGCCCCCGCGTACCAGCAAGGGCAGAACCAG |
| | | CTTTATAATGAGCTGAATCTTGGACGACGGGAGGAATAT |
| | | GACGTGCTTGACAAGAGGCGAGGTAGGGACCCTGAGAT |
| | | GGGGGGAAAACCTCGGAGGAAAAACCCACAGGAAGGC |
| | | CTGTATAACGAACTGCAGAAGGACAAGATGGCTGAAGC |
| | | CTACTCTGAGATTGGAATGAAAGGGGAACGCAGACGCG |
| | | GCAAGGGCCATGATGGCCTCTACCAAGGTCTAAGCACT |
| | | GCCACCAAGGACACCTATGACGCACTCCACATGCAAGC |
| | | TCTACCTCCCCGTTGATAAMC |
| MC0285- | SEQ ID | MALPVTALLLPLALLLHAARPQVQLVQSGGGVVQPGGSLR |
| 3PF12_8_P | NO: 12 | VSCAASGVTLSDYGMHWVRQAPGKGLEWMAFIRNDGSD |
| D1_HER2 | | KYYADSVKGRFTISRDNSKKTVSLQMSSLRAEDTAVYYCA |
| Protein | | KNGESGPLDYWYFDLWGRGTLVTVSSGGGGSGGGGSGGG |
| Sequence | | GSDIVMTQSPSFLSASVGDRVTITCRASHGINNYLAWYQQ |
| (VR285) | | KPGKAPKLLIYAASTLQSGVPSRFSGSGSGTEFTLTISSLQPE |
| | | DFATYYCQQYDSYPPTFGRGTKVEIKTTTPAPRPPTPAPTIA |
| | | SQPLSLRPEACRPAAGGAVHTRGLDFACDIYIWAPLAGTC |
| | | GVLLLSLVITLYCCSRAARGTIGARRTGQPLKEDPSAVPVF |
| | | SVDYGELDFQWREKTPEPPVPCVPEQTEYATIVFPSGMGTS |
| | | SPARRGSADGPRSAQPLRPEDGHCSWPLRRKRGSGEGRGS |
| | | LLTCGDVEENPGPMALPVTALLLPLALLLHAARPEVQLVE |
| | | SGGGLVQPGGSLRLSCAASGFNIKDTYIHWVRQAPGKGLE |
| | | WVARIYPTNGYTRYADSVKGRFTISADTSKNTAYLQMNSL |
| | | RAEDTAVYYCSRWGGDGFYAMDYWGQGTLVTVSSGSTS |
| | | GSGKPGSGEGSTKGDIQMTQSPSSLSASVGDRVTITCRASQ |
| | | DVNTAVAWYQQKPGKAPKLLIYSASFLYSGVPSRFSGSRS |
| | | GTDFTLTISSLQPEDFATYYCQQHYTTPPTFGQGTKVEIKIE |
| | | VMYPPPYLDNEKSNGTIIHVKGKHLCPSPLFPGPSKPFWVL |
| | | VVVGGVLACYSLLVTVAFIIFWVKRGRKKLLYIFKQPFMR |
| | | PVQTTQEEDGCSCRFPEEEEGGCELRVKFSRSADAPAYQQ |
| | | GQNQLYNELNLGRREEYDVLDKRRGRDPEMGGKPRRKNP |
| | | QEGLYNELQKDKMAEAYSEIGMKGERRRGKGHDGLYQG |
| | | LSTATKDTYDALHMQALPPR |
| MC0286- | SEQ ID | ATGGCACTGCCAGTGACCGCCCTGCTGCTGCCTCTGGCC |
| BB7.2_8_P | NO: 13 | CTGCTGCTGCACGCAGCCAGACCCCAGGTGCAGCTGCA |
| D1_EGFR | | GCAGTCTGGACCTGAGCTGGTGAAGCCAGGAGCCTCCG |
| nucleotide | | TGAAGATGTCTTGCAAGGCCAGCGGCTACACCTTCACAT |
| Sequence | | CTTATCACATCCAGTGGGTGAAGCAGCGGCCCGGACAG |
| (VR286) | | GGCCTGGAGTGGATCGGATGGATCTACCCAGGCGACGG |
| | | CTCCACACAGTATAACGAGAAGTTCAAGGGCAAGACCA |
| | | CACTGACCGCCGATAAGAGCAGCAGCACCGCCTACATG |
| | | CTGCTGAGCAGCCTGACCAGCGAGGACAGCGCCATCTA |
| | | CTTTTGCGCCAGGGAGGGCACATACTATGCTATGGACTA |
| | | TTGGGGCCAGGGCACCAGCGTGACAGTGTCTAGCGGAG |
| | | GAGGAGGCTCCGGAGGAGGAGGCTCTGGCGGCGGCGGC |
| | | AGCGACGTGCTGATGACCCAGACACCACTGAGCCTGCC |
| | | CGTGAGCCTGGGCGATCAGGTGAGCATCTCCTGTAGATC |
| | | CTCTCAGAGCATCGTGCACTCCAACGGCAATACCTACCT |
| | | GGAGTGGTATCTGCAGAAGCCAGGCCAGTCCCCCAAGC |
| | | TGCTGATCTATAAGGTGTCTAATCGGTTCAGCGGCGTGC |
| | | CTGACAGATTTTCTGGCAGCGGCTCCGGCACCGACTTCA |
| | | CCCTGAAGATCAGCCGGGTGGAGGCAGAGGATCTGGGC |
| | | GTGTACTATTGTTTCCAGGGCTCCCACGTGCCACGCACC |
| | | TTTGGCGGCGGTACCAAGCTGGAGATCAAGACTACGAC |
| | | CCCAGCACCTAGACCTCCCACCCCAGCTCCAACTATAGC |
| | | TTCCCAGCCATTGTCTCTCCGGCCAGAGGCGTGTCGACC |
| | | AGCCGCTGGAGGGGCCGTTCATACAAGAGGACTCGATT |
| | | TCGCTTGCGATATCTACATATGGGCCCCTCTTGCCGGGA |
| | | CATGCGGTGTCCTGCTTCTAAGCTTGGTTATTACCCTCTA |
| | | TTGCTGCAGCAGGGCCGCCCGCGGCACCATCGGCGCCA |
| | | GGCGCACAGGCCAGCCTCTGAAGGAGGACCCTTCCGCC |
| | | GTGCCAGTGTTCTCTGTGGACTACGGCGAGCTGGATTTT |
| | | CAGTGGCGGGAGAAAACCCCAGAGCCACCTGTGCCCTG |
| | | CGTGCCTGAGCAGACCGAGTATGCCACAATCGTGTTTCC |
| | | ATCCGGAATGGGCACAAGCTCCCCTGCAAGGAGAGGCA |
| | | GCGCCGACGGACCACGGTCCGCCCAGCCACTGCGGCCC |
| | | GAGGATGGCCACTGTTCTTGGCCCCTGCGGAGAAAGCGT |
| | | GGATCCGGGGAAGGCCGAGGCTCCCTTCTAACATGTGG |
| | | AGATGTCGAGGAAAACCCTGGCCCTATGGCGCTGCCAG |
| | | TCACTGCATTGTTATTGCCTCTGGCCCTGCTTCTCCATGC |
| | | GGCGCGCCCACAAGTGCAGCTGAAACAGAGCGGACCAG |
| | | GACTGGTTCAACCCAGCCAGAGCTTGAGCATCACGTGCA |
| | | CGGTTAGCGGCTTCAGTCTGACCAATTATGGTGTGCACT |
| | | GGGTGAGGCAGTCTCCAGGAAAGGGCCTGGAGTGGCTT |
| | | GGAGTCATTTGGAGCGGTGGGAATACAGATTACAATAC |
| | | ACCTTTTACGTCACGTCTCTCCATTAACAAGGACAACTC |
| | | CAAATCCCAAGTATTTTTCAAAATGAATAGCCTGCAGAG |
| | | TAATGATACCGCCATCTATTACTGTGCACGAGCTTTGAC |
| | | ATATTACGACTATGAATTTGCCTATTGGGGTCAAGGCAC |
| | | GCTGGTGACCGTATCAGGCTCAACATCCGGGTCCGGTAA |
| | | GCCGGGCTCCGGCGAGGGGTCTACAAAGGGAGACATCC |
| | | TTCTGACACAGAGCCCCGTGATCCTGTCCGTGTCCCCCG |
| | | GCGAGAGAGTATCATTTTCCTGTAGGGCTTCTCAGAGCA |
| | | TCGGAACAAATATCCACTGGTATCAGCAACGGACTAAC |
| | | GGATCACCTCGCCTGCTCATAAAGTACGCCAGTGAATCT |
| | | ATTAGTGGCATACCGAGCCGCTTCAGCGGGAGTGGCTCC |
| | | GGCACAGACTTTACTCTGAGTATAAATTCCGTGGAATCT |
| | | GAGGACATCGCGGACTATTACTGCCAGCAAAACAATAA |
| | | CTGGCCCACCACGTTCGGCGCGGGAACTAAACTAGAAC |
| | | TAAAGATTGAAGTTATGTATCCTCCTCCTTACCTAGACA |
| | | ATGAGAAGAGCAATGGAACCATTATCCATGTGAAAGGG |
| | | AAACACCTTTGTCCAAGTCCCCTATTTCCCGGGCCTTCG |
| | | AAGCCCTTTTGGGTGCTGGTGGTGGTTGGTGGAGTCCTG |
| | | GCTTGCTATAGCTTGCTAGTAACAGTAGCGTTTATTATTT |
| | | TCTGGGTGAAACGCGGCCGCAAGAAACTGCTCTACATCT |
| | | TTAAACAGCCGTTCATGAGGCCTGTGCAGACAACGCAG |
| | | GAAGAGGATGGCTGTAGTTGTCGGTTTCCGGAAGAGGA |
| | | AGAGGGGGGCTGCGAGTTGCGTGTCAAATTTTCTCGGTC |
| | | TGCCGACGCCCCCGCGTACCAGCAAGGGCAGAACCAGC |
| | | TTTATAATGAGCTGAATCTTGGACGACGGGAGGAATATG |
| | | ACGTGCTTGACAAGAGGCGAGGTAGGGACCCTGAGATG |
| | | GGGGGAAAACCTCGGAGGAAAAACCCACAGGAAGGCCT |
| | | GTATAACGAACTGCAGAAGGACAAGATGGCTGAAGCCT |
| | | ACTCTGAGATTGGAATGAAAGGGGAACGCAGACGCGGC |
| | | AAGGGCCATGATGGCCTCTACCAAGGTCTAAGCACTGCC |
| | | ACCAAGGACACCTATGACGCACTCCACATGCAAGCTCTA |
| | | CCTCCCCGTTGATAA |
| MC0286- | SEQ ID | MALPVTALLLPLALLLHAARPQVQLQQSGPELVKPGASVK |
| BB7.2_8_P | NO: 14 | MSCKASGYTFTSYHIQWVKQRPGQGLEWIGWIYPGDGSTQ |
| D1_EGFR | | YNEKFKGKTTLTADKSSSTAYMLLSSLTSEDSAIYFCAREG |
| Protein | | TYYAMDYWGQGTSVTVSSGGGGSGGGGSGGGGSDVLMT |
| Sequence | | QTPLSLPVSLGDQVSISCRSSQSIVHSNGNTYLEWYLQKPG |
| (VR286) | | QSPKLLIYKVSNRFSGVPDRFSGSGSGTDFTLKISRVEAEDL |
| | | GVYYCFQGSHVPRTFGGGTKLEIKTTTPAPRPPTPAPTIASQ |
| | | PLSLRPEACRPAAGGAVHTRGLDFACDIYIWAPLAGTCGV |
| | | LLLSLVITLYCCSRAARGTIGARRTGQPLKEDPSAVPVFSV |
| | | DYGELDFQWREKTPEPPVPCVPEQTEYATIVFPSGMGTSSP |
| | | ARRGSADGPRSAQPLRPEDGHCSWPLRRKRGSGEGRGSLL |
| | | TCGDVEENPGPMALPVTALLLPLALLLHAARPQVQLKQSG |
| | | PGLVQPSQSLSITCTVSGFSLTNYGVHWVRQSPGKGLEWL |
| | | GVIWSGGNTDYNTPFTSRLSINKDNSKSQVFFKMNSLQSN |
| | | DTAIYYCARALTYYDYEFAYWGQGTLVTVSGSTSGSGKPG |
| | | SGEGSTKGDILLTQSPVILSVSPGERVSFSCRASQSIGTNIHW |
| | | YQQRTNGSPRLLIKYASESISGIPSRFSGSGSGTDFTLSINSV |
| | | ESEDIADYYCQQNNNWPTTFGAGTKLELKIEVMYPPPYLD |
| | | NEKSNGTIIHVKGKHLCPSPLFPGPSKPFWVLVVVGGVLAC |
| | | YSLLVTVAFIIFWVKRGRKKLLYIFKQPFMRPVQTTQEEDG |
| | | CSCRFPEEEEGGCELRVKFSRSADAPAYQQGQNQLYNELN |
| | | LGRREEYDVLDKRRGRDPEMGGKPRRKNPQEGLYNELQK |
| | | DKMAEAYSEIGMKGERRRGKGHDGLYQGLSTATKDTYDA |
| | | LHMQALPPR |
| MC0287- | SEQ ID | ATGGCACTGCCAGTGACCGCCCTGCTGCTGCCTCTGGCC |
| 3PF12_8_P | NO: 15 | CTGCTGCTGCACGCAGCCAGACCCCAAGTGCAACTAGTC |
| D1_EGFR | | CAATCAGGTGGAGGCGTCGTGCAACCTGGAGGGTCCCT |
| Nucleotide | | CCGCGTTAGCTGCGCCGCATCAGGCGTTACCTTGTCAGA |
| Sequence | | CTACGGCATGCATTGGGTTAGGCAAGCCCCCGGCAAGG |
| (VR287) | | GGCTCGAATGGATGGCTTTCATTCGGAATGACGGGAGC |
| | | GATAAATATTACGCGGATTCAGTTAAAGGGCGGTTCACC |
| | | ATCAGCCGCGACAATAGCAAAAAGACGGTCTCCTTACA |
| | | GATGTCCAGCTTGCGGGCCGAAGACACGGCTGTATACTA |
| | | TTGTGCTAAAAATGGCGAGAGCGGCCCCCTGGATTACTG |
| | | GTACTTTGACCTGTGGGGCAGAGGCACCCTGGTCACGGT |
| | | GTCCTCTGGAGGAGGAGGCTCCGGAGGAGGAGGCTCTG |
| | | GCGGCGGCGGCAGCGACATTGTAATGACCCAGTCACCC |
| | | TCCTTCCTTAGTGCCTCAGTCGGAGACCGCGTGACTATC |
| | | ACTTGTCGTGCCTCACACGGAATTAATAACTACCTCGCT |
| | | TGGTATCAGCAAAAACCAGGCAAGGCCCCGAAACTATT |
| | | GATCTACGCCGCATCTACTCTGCAGAGCGGAGTACCGAG |
| | | CAGATTTAGTGGTTCCGGCAGCGGAACCGAGTTCACCCT |
| | | AACCATTTCCAGTTTGCAGCCAGAGGATTTCGCGACCTA |
| | | TTACTGCCAGCAATACGATTCATACCCGCCAACTTTCGG |
| | | AAGAGGTACCAAGGTTGAAATCAAGACTACGACCCCAG |
| | | CACCTAGACCTCCCACCCCAGCTCCAACTATAGCTTCCC |
| | | AGCCATTGTCTCTCCGGCCAGAGGCGTGTCGACCAGCCG |
| | | CTGGAGGGGCCGTTCATACAAGAGGACTCGATTTCGCTT |
| | | GCGATATCTACATATGGGCCCCTCTTGCCGGGACATGCG |
| | | GTGTCCTGCTTCTAAGCTTGGTTATTACCCTCTATTGCTG |
| | | CAGCAGGGCCGCCCGCGGCACCATCGGCGCCAGGCGCA |
| | | CAGGCCAGCCTCTGAAGGAGGACCCTTCCGCCGTGCCA |
| | | GTGTTCTCTGTGGACTACGGCGAGCTGGATTTTCAGTGG |
| | | CGGGAGAAAACCCCAGAGCCACCTGTGCCCTGCGTGCC |
| | | TGAGCAGACCGAGTATGCCACAATCGTGTTTCCATCCGG |
| | | AATGGGCACAAGCTCCCCTGCAAGGAGAGGCAGCGCCG |
| | | ACGGACCACGGTCCGCCCAGCCACTGCGGCCCGAGGAT |
| | | GGCCACTGTTCTTGGCCCCTGCGGAGAAAGCGTGGATCC |
| | | GGGGAAGGCCGAGGCTCCCTTCTAACATGTGGAGATGT |
| | | CGAGGAAAACCCTGGCCCTATGGCGCTGCCAGTCACTGC |
| | | ATTGTTATTGCCTCTGGCCCTGCTTCTCCATGCGGCGCGC |
| | | CCACAAGTGCAGCTGAAACAGAGCGGACCAGGACTGGT |
| | | TCAACCCAGCCAGAGCTTGAGCATCACGTGCACGGTTAG |
| | | CGGCTTCAGTCTGACCAATTATGGTGTGCACTGGGTGAG |
| | | GCAGTCTCCAGGAAAGGGCCTGGAGTGGCTTGGAGTCA |
| | | TTTGGAGCGGTGGGAATACAGATTACAATACACCTTTTA |
| | | CGTCACGTCTCTCCATTAACAAGGACAACTCCAAATCCC |
| | | AAGTATTTTTCAAAATGAATAGCCTGCAGAGTAATGATA |
| | | CCGCCATCTATTACTGTGCACGAGCTTTGACATATTACG |
| | | ACTATGAATTTGCCTATTGGGGTCAAGGCACGCTGGTGA |
| | | CCGTATCAGGCTCAACATCCGGGTCCGGTAAGCCGGGCT |
| | | CCGGCGAGGGGTCTACAAAGGGAGACATCCTTCTGACA |
| | | CAGAGCCCCGTGATCCTGTCCGTGTCCCCCGGCGAGAGA |
| | | GTATCATTTTCCTGTAGGGCTTCTCAGAGCATCGGAACA |
| | | AATATCCACTGGTATCAGCAACGGACTAACGGATCACCT |
| | | CGCCTGCTCATAAAGTACGCCAGTGAATCTATTAGTGGC |
| | | ATACCGAGCCGCTTCAGCGGGAGTGGCTCCGGCACAGA |
| | | CTTTACTCTGAGTATAAATTCCGTGGAATCTGAGGACAT |
| | | CGCGGACTATTACTGCCAGCAAAACAATAACTGGCCCA |
| | | CCACGTTCGGCGCGGGAACTAAACTAGAACTAAAGATT |
| | | GAAGTTATGTATCCTCCTCCTTACCTAGACAATGAGAAG |
| | | AGCAATGGAACCATTATCCATGTGAAAGGGAAACACCT |
| | | TTGTCCAAGTCCCCTATTTCCCGGGCCTTCGAAGCCCTTT |
| | | TGGGTGCTGGTGGTGGTTGGTGGAGTCCTGGCTTGCTAT |
| | | AGCTTGCTAGTAACAGTAGCGTTTATTATTTTCTGGGTG |
| | | AAACGCGGCCGCAAGAAACTGCTCTACATCTTTAAACA |
| | | GCCGTTCATGAGGCCTGTGCAGACAACGCAGGAAGAGG |
| | | ATGGCTGTAGTTGTCGGTTTCCGGAAGAGGAAGAGGGG |
| | | GGCTGCGAGTTGCGTGTCAAATTTTCTCGGTCTGCCGAC |
| | | GCCCCCGCGTACCAGCAAGGGCAGAACCAGCTTTATAA |
| | | TGAGCTGAATCTTGGACGACGGGAGGAATATGACGTGC |
| | | TTGACAAGAGGCGAGGTAGGGACCCTGAGATGGGGGGA |
| | | AAACCTCGGAGGAAAAACCCACAGGAAGGCCTGTATAA |
| | | CGAACTGCAGAAGGACAAGATGGCTGAAGCCTACTCTG |
| | | AGATTGGAATGAAAGGGGAACGCAGACGCGGCAAGGG |
| | | CCATGATGGCCTCTACCAAGGTCTAAGCACTGCCACCAA |
| | | GGACACCTATGACGCACTCCACATGCAAGCTCTACCTCC |
| | | CCGTTGATAA |
| MC0287- | SEQ ID | MALPVTALLLPLALLLHAARPQVQLVQSGGGVVQPGGSLR |
| 3PF12_8_P | NO: 16 | VSCAASGVTLSDYGMHWVRQAPGKGLEWMAFIRNDGSD |
| D1_EGFR | | KYYADSVKGRFTISRDNSKKTVSLQMSSLRAEDTAVYYCA |
| Protein | | KNGESGPLDYWYFDLWGRGTLVTVSSGGGGSGGGGSGGG |
| Sequence | | GSDIVMTQSPSFLSASVGDRVTITCRASHGINNYLAWYQQ |
| (VR287) | | KPGKAPKLLIYAASTLQSGVPSRFSGSGSGTEFTLTISSLQPE |
| | | DFATYYCQQYDSYPPTFGRGTKVEIKTTTPAPRPPTPAPTIA |
| | | SQPLSLRPEACRPAAGGAVHTRGLDFACDIYIWAPLAGTC |
| | | GVLLLSLVITLYCCSRAARGTIGARRTGQPLKEDPSAVPVF |
| | | SVDYGELDFQWREKTPEPPVPCVPEQTEYATIVFPSGMGTS |
| | | SPARRGSADGPRSAQPLRPEDGHCSWPLRRKRGSGEGRGS |
| | | LLTCGDVEENPGPMALPVTALLLPLALLLHAARPQVQLKQ |
| | | SGPGLVQPSQSLSITCTVSGFSLTNYGVHWVRQSPGKGLE |
| | | WLGVIWSGGNTDYNTPFTSRLSINKDNSKSQVFFKMNSLQ |
| | | SNDTAIYYCARALTYYDYEFAYWGQGTLVTVSGSTSGSGK |
| | | PGSGEGSTKGDILLTQSPVILSVSPGERVSFSCRASQSIGTNI |
| | | HWYQQRTNGSPRLLIKYASESISGIPSRFSGSGSGTDFTLSIN |
| | | SVESEDIADYYCQQNNNWPTTFGAGTKLELKIEVMYPPPY |
| | | LDNEKSNGTIIHVKGKHLCPSPLFPGPSKPFWVLVVVGGVL |
| | | ACYSLLVTVAFIIFWVKRGRKKLLYIFKQPFMRPVQTTQEE |
| | | DGCSCRFPEEEEGGCELRVKFSRSADAPAYQQGQNQLYNE |
| | | LNLGRREEYDVLDKRRGRDPEMGGKPRRKNPQEGLYNEL |
| | | QKDKMAEAYSEIGMKGERRRGKGHDGLYQGLSTATKDTY |
| | | DALHMQALPPR |
| MC0288- | SEQ ID | ATGGCACTGCCAGTGACCGCCCTGCTGCTGCCTCTGGCC |
| BB7.2_28_ | NO: 17 | CTGCTGCTGCACGCAGCCAGACCCCAGGTGCAGCTGCA |
| Pdel_HER2 | | GCAGTCTGGACCTGAGCTGGTGAAGCCAGGAGCCTCCG |
| Nucleotide | | TGAAGATGTCTTGCAAGGCCAGCGGCTACACCTTCACAT |
| Sequence | | CTTATCACATCCAGTGGGTGAAGCAGCGGCCCGGACAG |
| (VR288) | | GGCCTGGAGTGGATCGGATGGATCTACCCAGGCGACGG |
| | | CTCCACACAGTATAACGAGAAGTTCAAGGGCAAGACCA |
| | | CACTGACCGCCGATAAGAGCAGCAGCACCGCCTACATG |
| | | CTGCTGAGCAGCCTGACCAGCGAGGACAGCGCCATCTA |
| | | CTTTTGCGCCAGGGAGGGCACATACTATGCTATGGACTA |
| | | TTGGGGCCAGGGCACCAGCGTGACAGTGTCTAGCGGAG |
| | | GAGGAGGCTCCGGAGGAGGAGGCTCTGGCGGCGGCGGC |
| | | AGCGACGTGCTGATGACCCAGACACCACTGAGCCTGCC |
| | | CGTGAGCCTGGGCGATCAGGTGAGCATCTCCTGTAGATC |
| | | CTCTCAGAGCATCGTGCACTCCAACGGCAATACCTACCT |
| | | GGAGTGGTATCTGCAGAAGCCAGGCCAGTCCCCCAAGC |
| | | TGCTGATCTATAAGGTGTCTAATCGGTTCAGCGGCGTGC |
| | | CTGACAGATTTTCTGGCAGCGGCTCCGGCACCGACTTCA |
| | | CCCTGAAGATCAGCCGGGTGGAGGCAGAGGATCTGGGC |
| | | GTGTACTATTGTTTCCAGGGCTCCCACGTGCCACGCACC |
| | | TTTGGCGGCGGTACCAAGCTGGAGATCAAGATTGAAGTT |
| | | ATGTATCCTCCTCCTTACCTAGACAATGAGAAGAGCAAT |
| | | GGAACCATTATCCATGTGAAAGGGAAACACCTTTGTCCA |
| | | AGTCCCCTATTTCCCGGGCCTTCGAAGCCCTTTTGGGTG |
| | | CTGGTGGTGGTTGGTGGAGTCCTGGCTTGCTATAGCTTG |
| | | CTAGTAACAGTAGCGTTTATTATTTTCTGGGTGCGGAGA |
| | | AAGCGTGGATCCGGGGAAGGCCGAGGCTCCCTTCTAAC |
| | | ATGTGGAGATGTCGAGGAAAACCCTGGCCCTATGGCGC |
| | | TGCCAGTCACTGCATTGTTATTGCCTCTGGCCCTGCTTCT |
| | | CCATGCGGCGCGCCCAGAAGTGCAGCTGGTCGAGAGCG |
| | | GAGGCGGACTGGTTCAACCCGGAGGCAGCTTGAGACTG |
| | | TCCTGCGCGGCCAGCGGCTTCAACATCAAGGATACCTAT |
| | | ATCCACTGGGTGAGGCAGGCTCCAGGAAAGGGCCTGGA |
| | | GTGGGTGGCAAGGATTTACCCTACTAATGGATATACACG |
| | | CTACGCTGATTCCGTGAAGGGACGCTTTACAATCTCAGC |
| | | AGATACATCCAAAAACACGGCCTATTTACAGATGAATA |
| | | GTTTGCGGGCCGAAGACACGGCTGTATACTATTGTTCTC |
| | | GGTGGGGGGGCGATGGATTTTATGCGATGGATTACTGG |
| | | GGCCAGGGCACCCTGGTAACCGTGTCAAGCGGCTCAAC |
| | | ATCCGGGTCCGGTAAGCCGGGCTCCGGCGAGGGGTCTA |
| | | CAAAGGGAGATATACAGATGACACAGTCCCCCAGTTCC |
| | | CTGTCCGCCTCAGTGGGAGACCGAGTGACGATTACCTGT |
| | | CGTGCCAGCCAGGACGTCAATACCGCCGTCGCTTGGTAT |
| | | CAGCAAAAACCAGGCAAGGCCCCGAAACTATTGATCTA |
| | | CAGTGCCTCTTTTCTGTACTCCGGGGTGCCGAGCAGATT |
| | | TAGTGGCTCCAGGAGCGGAACCGATTTCACCCTAACCAT |
| | | TTCCAGTTTGCAGCCAGAGGATTTCGCGACCTATTACTG |
| | | CCAGCAACACTACACCACACCGCCAACTTTCGGACAAG |
| | | GAACCAAGGTTGAAATCAAAACTACGACCCCAGCACCT |
| | | AGACCTCCCACCCCAGCTCCAACTATAGCTTCCCAGCCA |
| | | TTGTCTCTCCGGCCAGAGGCGTGTCGACCAGCCGCTGGA |
| | | GGGGCCGTTCATACAAGAGGACTCGATTTCGCTTGCGAT |
| | | ATCTACATATGGGCCCCTCTTGCCGGGACATGCGGTGTC |
| | | CTGCTTCTAAGCTTGGTTATTACCCTCTATTGCAAACGCG |
| | | GCCGCAAGAAACTGCTCTACATCTTTAAACAGCCGTTCA |
| | | TGAGGCCTGTGCAGACAACGCAGGAAGAGGATGGCTGT |
| | | AGTTGTCGGTTTCCGGAAGAGGAAGAGGGGGGCTGCGA |
| | | GTTGCGTGTCAAATTTTCTCGGTCTGCCGACGCCCCCGC |
| | | GTACCAGCAAGGGCAGAACCAGCTTTATAATGAGCTGA |
| | | ATCTTGGACGACGGGAGGAATATGACGTGCTTGACAAG |
| | | AGGCGAGGTAGGGACCCTGAGATGGGGGGAAAACCTCG |
| | | GAGGAAAAACCCACAGGAAGGCCTGTATAACGAACTGC |
| | | AGAAGGACAAGATGGCTGAAGCCTACTCTGAGATTGGA |
| | | ATGAAAGGGGAACGCAGACGCGGCAAGGGCCATGATGG |
| | | CCTCTACCAAGGTCTAAGCACTGCCACCAAGGACACCTA |
| | | TGACGCACTCCACATGCAAGCTCTACCTCCCCGTTGATA |
| | | A |
| MC0288- | SEQ ID | MALPVTALLLPLALLLHAARPQVQLQQSGPELVKPGASVK |
| BB7.2_28_ | NO: 18 | MSCKASGYTFTSYHIQWVKQRPGQGLEWIGWIYPGDGSTQ |
| Pdel_HER2 | | YNEKFKGKTTLTADKSSSTAYMLLSSLTSEDSAIYFCAREG |
| Protein | | TYYAMDYWGQGTSVTVSSGGGGSGGGGSGGGGSDVLMT |
| Sequence | | QTPLSLPVSLGDQVSISCRSSQSIVHSNGNTYLEWYLQKPG |
| (VR288) | | QSPKLLIYKVSNRFSGVPDRFSGSGSGTDFTLKISRVEAEDL |
| | | GVYYCFQGSHVPRTFGGGTKLEIKIEVMYPPPYLDNEKSN |
| | | GTIIHVKGKHLCPSPLFPGPSKPFWVLVVVGGVLACYSLLV |
| | | TVAFIIFWVRRKRGSGEGRGSLLTCGDVEENPGPMALPVT |
| | | ALLLPLALLLHAARPEVQLVESGGGLVQPGGSLRLSCAAS |
| | | GFNIKDTYIHWVRQAPGKGLEWVARIYPTNGYTRYADSV |
| | | KGRFTISADTSKNTAYLQMNSLRAEDTAVYYCSRWGGDG |
| | | FYAMDYWGQGTLVTVSSGSTSGSGKPGSGEGSTKGDIQM |
| | | TQSPSSLSASVGDRVTITCRASQDVNTAVAWYQQKPGKAP |
| | | KLLIYSASFLYSGVPSRFSGSRSGTDFTLTISSLQPEDFATYY |
| | | CQQHYTTPPTFGQGTKVEIKTTTPAPRPPTPAPTIASQPLSL |
| | | RPEACRPAAGGAVHTRGLDFACDIYIWAPLAGTCGVLLLS |
| | | LVITLYCKRGRKKLLYIFKQPFMRPVQTTQEEDGCSCRFPE |
| | | EEEGGCELRVKFSRSADAPAYQQGQNQLYNELNLGRREEY |
| | | DVLDKRRGRDPEMGGKPRRKNPQEGLYNELQKDKMAEA |
| | | YSEIGMKGERRRGKGHDGLYQGLSTATKDTYDALHMQAL |
| | | PPR |
| MC0289- | SEQ ID | ATGGCACTGCCAGTGACCGCCCTGCTGCTGCCTCTGGCC |
| 3PF12_28_ | NO: 19 | CTGCTGCTGCACGCAGCCAGACCCCAAGTGCAACTAGTC |
| Pdel_HER2 | | CAATCAGGTGGAGGCGTCGTGCAACCTGGAGGGTCCCT |
| Nucleotide | | CCGCGTTAGCTGCGCCGCATCAGGCGTTACCTTGTCAGA |
| Sequence | | CTACGGCATGCATTGGGTTAGGCAAGCCCCCGGCAAGG |
| (VR289) | | GGCTCGAATGGATGGCTTTCATTCGGAATGACGGGAGC |
| | | GATAAATATTACGCGGATTCAGTTAAAGGGCGGTTCACC |
| | | ATCAGCCGCGACAATAGCAAAAAGACGGTCTCCTTACA |
| | | GATGTCCAGCTTGCGGGCCGAAGACACGGCTGTATACTA |
| | | TTGTGCTAAAAATGGCGAGAGCGGCCCCCTGGATTACTG |
| | | GTACTTTGACCTGTGGGGCAGAGGCACCCTGGTCACGGT |
| | | GTCCTCTGGAGGAGGAGGCTCCGGAGGAGGAGGCTCTG |
| | | GCGGCGGCGGCAGCGACATTGTAATGACCCAGTCACCC |
| | | TCCTTCCTTAGTGCCTCAGTCGGAGACCGCGTGACTATC |
| | | ACTTGTCGTGCCTCACACGGAATTAATAACTACCTCGCT |
| | | TGGTATCAGCAAAAACCAGGCAAGGCCCCGAAACTATT |
| | | GATCTACGCCGCATCTACTCTGCAGAGCGGAGTACCGAG |
| | | CAGATTTAGTGGTTCCGGCAGCGGAACCGAGTTCACCCT |
| | | AACCATTTCCAGTTTGCAGCCAGAGGATTTCGCGACCTA |
| | | TTACTGCCAGCAATACGATTCATACCCGCCAACTTTCGG |
| | | AAGAGGTACCAAGGTTGAAATCAAGATTGAAGTTATGT |
| | | ATCCTCCTCCTTACCTAGACAATGAGAAGAGCAATGGAA |
| | | CCATTATCCATGTGAAAGGGAAACACCTTTGTCCAAGTC |
| | | CCCTATTTCCCGGGCCTTCGAAGCCCTTTTGGGTGCTGGT |
| | | GGTGGTTGGTGGAGTCCTGGCTTGCTATAGCTTGCTAGT |
| | | AACAGTAGCGTTTATTATTTTCTGGGTGCGGAGAAAGCG |
| | | TGGATCCGGGGAAGGCCGAGGCTCCCTTCTAACATGTGG |
| | | AGATGTCGAGGAAAACCCTGGCCCTATGGCGCTGCCAG |
| | | TCACTGCATTGTTATTGCCTCTGGCCCTGCTTCTCCATGC |
| | | GGCGCGCCCAGAAGTGCAGCTGGTCGAGAGCGGAGGCG |
| | | GACTGGTTCAACCCGGAGGCAGCTTGAGACTGTCCTGCG |
| | | CGGCCAGCGGCTTCAACATCAAGGATACCTATATCCACT |
| | | GGGTGAGGCAGGCTCCAGGAAAGGGCCTGGAGTGGGTG |
| | | GCAAGGATTTACCCTACTAATGGATATACACGCTACGCT |
| | | GATTCCGTGAAGGGACGCTTTACAATCTCAGCAGATACA |
| | | TCCAAAAACACGGCCTATTTACAGATGAATAGTTTGCGG |
| | | GCCGAAGACACGGCTGTATACTATTGTTCTCGGTGGGGG |
| | | GGCGATGGATTTTATGCGATGGATTACTGGGGCCAGGGC |
| | | ACCCTGGTAACCGTGTCAAGCGGCTCAACATCCGGGTCC |
| | | GGTAAGCCGGGCTCCGGCGAGGGGTCTACAAAGGGAGA |
| | | TATACAGATGACACAGTCCCCCAGTTCCCTGTCCGCCTC |
| | | AGTGGGAGACCGAGTGACGATTACCTGTCGTGCCAGCC |
| | | AGGACGTCAATACCGCCGTCGCTTGGTATCAGCAAAAA |
| | | CCAGGCAAGGCCCCGAAACTATTGATCTACAGTGCCTCT |
| | | TTTCTGTACTCCGGGGTGCCGAGCAGATTTAGTGGCTCC |
| | | AGGAGCGGAACCGATTTCACCCTAACCATTTCCAGTTTG |
| | | CAGCCAGAGGATTTCGCGACCTATTACTGCCAGCAACAC |
| | | TACACCACACCGCCAACTTTCGGACAAGGAACCAAGGT |
| | | TGAAATCAAAACTACGACCCCAGCACCTAGACCTCCCAC |
| | | CCCAGCTCCAACTATAGCTTCCCAGCCATTGTCTCTCCG |
| | | GCCAGAGGCGTGTCGACCAGCCGCTGGAGGGGCCGTTC |
| | | ATACAAGAGGACTCGATTTCGCTTGCGATATCTACATAT |
| | | GGGCCCCTCTTGCCGGGACATGCGGTGTCCTGCTTCTAA |
| | | GCTTGGTTATTACCCTCTATTGCAAACGCGGCCGCAAGA |
| | | AACTGCTCTACATCTTTAAACAGCCGTTCATGAGGCCTG |
| | | TGCAGACAACGCAGGAAGAGGATGGCTGTAGTTGTCGG |
| | | TTTCCGGAAGAGGAAGAGGGGGGCTGCGAGTTGCGTGT |
| | | CAAATTTTCTCGGTCTGCCGACGCCCCCGCGTACCAGCA |
| | | AGGGCAGAACCAGCTTTATAATGAGCTGAATCTTGGAC |
| | | GACGGGAGGAATATGACGTGCTTGACAAGAGGCGAGGT |
| | | AGGGACCCTGAGATGGGGGGAAAACCTCGGAGGAAAA |
| | | ACCCACAGGAAGGCCTGTATAACGAACTGCAGAAGGAC |
| | | AAGATGGCTGAAGCCTACTCTGAGATTGGAATGAAAGG |
| | | GGAACGCAGACGCGGCAAGGGCCATGATGGCCTCTACC |
| | | AAGGTCTAAGCACTGCCACCAAGGACACCTATGACGCA |
| | | CTCCACATGCAAGCTCTACCTCCCCGTTGATAA |
| MC0289- | SEQ ID | MALPVTALLLPLALLLHAARPQVQLVQSGGGVVQPGGSLR |
| 3PF12_28_ | NO: 20 | VSCAASGVTLSDYGMHWVRQAPGKGLEWMAFIRNDGSD |
| Pdel_HER2 | | KYYADSVKGRFTISRDNSKKTVSLQMSSLRAEDTAVYYCA |
| Protein | | KNGESGPLDYWYFDLWGRGTLVTVSSGGGGSGGGGSGGG |
| Sequence | | GSDIVMTQSPSFLSASVGDRVTITCRASHGINNYLAWYQQ |
| (VR289) | | KPGKAPKLLIYAASTLQSGVPSRFSGSGSGTEFTLTISSLQPE |
| | | DFATYYCQQYDSYPPTFGRGTKVEIKIEVMYPPPYLDNEKS |
| | | NGTIIHVKGKHLCPSPLFPGPSKPFWVLVVVGGVLACYSLL |
| | | VTVAFIIFWVRRKRGSGEGRGSLLTCGDVEENPGPMALPV |
| | | TALLLPLALLLHAARPEVQLVESGGGLVQPGGSLRLSCAAS |
| | | GFNIKDTYIHWVRQAPGKGLEWVARIYPTNGYTRYADSV |
| | | KGRFTISADTSKNTAYLQMNSLRAEDTAVYYCSRWGGDG |
| | | FYAMDYWGQGTLVTVSSGSTSGSGKPGSGEGSTKGDIQM |
| | | TQSPSSLSASVGDRVTITCRASQDVNTAVAWYQQKPGKAP |
| | | KLLIYSASFLYSGVPSRFSGSRSGTDFTLTISSLQPEDFATYY |
| | | CQQHYTTPPTFGQGTKVEIKTTTPAPRPPTPAPTIASQPLSL |
| | | RPEACRPAAGGAVHTRGLDFACDIYIWAPLAGTCGVLLLS |
| | | LVITLYCKRGRKKLLYIFKQPFMRPVQTTQEEDGCSCRFPE |
| | | EEEGGCELRVKFSRSADAPAYQQGQNQLYNELNLGRREEY |
| | | DVLDKRRGRDPEMGGKPRRKNPQEGLYNELQKDKMAEA |
| | | YSEIGMKGERRRGKGHDGLYQGLSTATKDTYDALHMQAL |
| | | PPR |
| MC0290- | SEQ ID | ATGGCACTGCCAGTGACCGCCCTGCTGCTGCCTCTGGCC |
| 3PF12_28_ | NO: 21 | CTGCTGCTGCACGCAGCCAGACCCCAAGTGCAACTAGTC |
| LIR1_HER | | CAATCAGGTGGAGGCGTCGTGCAACCTGGAGGGTCCCT |
| 2 Nucleic | | CCGCGTTAGCTGCGCCGCATCAGGCGTTACCTTGTCAGA |
| acid | | CTACGGCATGCATTGGGTTAGGCAAGCCCCCGGCAAGG |
| sequence | | GGCTCGAATGGATGGCTTTCATTCGGAATGACGGGAGC |
| (VR290) | | GATAAATATTACGCGGATTCAGTTAAAGGGCGGTTCACC |
| | | ATCAGCCGCGACAATAGCAAAAAGACGGTCTCCTTACA |
| | | GATGTCCAGCTTGCGGGCCGAAGACACGGCTGTATACTA |
| | | TTGTGCTAAAAATGGCGAGAGCGGCCCCCTGGATTACTG |
| | | GTACTTTGACCTGTGGGGCAGAGGCACCCTGGTCACGGT |
| | | GTCCTCTGGAGGAGGAGGCTCCGGAGGAGGAGGCTCTG |
| | | GCGGCGGCGGCAGCGACATTGTAATGACCCAGTCACCC |
| | | TCCTTCCTTAGTGCCTCAGTCGGAGACCGCGTGACTATC |
| | | ACTTGTCGTGCCTCACACGGAATTAATAACTACCTCGCT |
| | | TGGTATCAGCAAAAACCAGGCAAGGCCCCGAAACTATT |
| | | GATCTACGCCGCATCTACTCTGCAGAGCGGAGTACCGAG |
| | | CAGATTTAGTGGTTCCGGCAGCGGAACCGAGTTCACCCT |
| | | AACCATTTCCAGTTTGCAGCCAGAGGATTTCGCGACCTA |
| | | TTACTGCCAGCAATACGATTCATACCCGCCAACTTTCGG |
| | | AAGAGGTACCAAGGTTGAAATCAAGATTGAAGTTATGT |
| | | ATCCTCCTCCTTACCTAGACAATGAGAAGAGCAATGGAA |
| | | CCATTATCCATGTGAAAGGGAAACACCTTTGTCCAAGTC |
| | | CCCTATTTCCCGGGCCTTCGAAGCCCTTTTGGGTGCTGGT |
| | | GGTGGTTGGTGGAGTCCTGGCTTGCTATAGCTTGCTAGT |
| | | AACAGTAGCGTTTATTATTTTCTGGGTGCTGCGCCACAG |
| | | GAGACAGGGCAAGCACTGGACCAGCACCCAGCGGAAGG |
| | | CCGACTTTCAGCACCCTGCCGGCGCCGTGGGCCCTGAGC |
| | | CTACCGACAGGGGCCTGCAGTGGAGGAGCTCCCCAGCC |
| | | GCCGATGCCCAGGAGGAGAATCTGTACGCCGCCGTGAA |
| | | GCACACCCAGCCAGAGGACGGCGTGGAGATGGACACCC |
| | | GCTCCCCACACGACGAGGATCCACAGGCCGTGACCTAC |
| | | GCCGAGGTGAAGCACAGCCGCCCCAGACGCGAGATGGC |
| | | CAGCCCACCCAGCCCCCTGTCCGGCGAGTTCCTGGACAC |
| | | CAAGGACAGGCAGGCCGAGGAGGACCGGCAGATGGAC |
| | | ACCGAGGCCGCCGCCTCCGAGGCCCCCCAGGACGTGAC |
| | | CTACGCCCAGCTGCACTCCCTGACCCTGCGGAGAGAGGC |
| | | CACCGAGCCCCCACCCAGCCAGGAGGGCCCCTCCCCCG |
| | | CCGTGCCTAGCATCTACGCCACCCTGGCCATCCACCGGA |
| | | GAAAGCGTGGATCCGGGGAAGGCCGAGGCTCCCTTCTA |
| | | ACATGTGGAGATGTCGAGGAAAACCCTGGCCCTATGGC |
| | | GCTGCCAGTCACTGCATTGTTATTGCCTCTGGCCCTGCTT |
| | | CTCCATGCGGCGCGCCCAGAAGTGCAGCTGGTCGAGAG |
| | | CGGAGGCGGACTGGTTCAACCCGGAGGCAGCTTGAGAC |
| | | TGTCCTGCGCGGCCAGCGGCTTCAACATCAAGGATACCT |
| | | ATATCCACTGGGTGAGGCAGGCTCCAGGAAAGGGCCTG |
| | | GAGTGGGTGGCAAGGATTTACCCTACTAATGGATATACA |
| | | CGCTACGCTGATTCCGTGAAGGGACGCTTTACAATCTCA |
| | | GCAGATACATCCAAAAACACGGCCTATTTACAGATGAA |
| | | TAGTTTGCGGGCCGAAGACACGGCTGTATACTATTGTTC |
| | | TCGGTGGGGGGGCGATGGATTTTATGCGATGGATTACTG |
| | | GGGCCAGGGCACCCTGGTAACCGTGTCAAGCGGCTCAA |
| | | CATCCGGGTCCGGTAAGCCGGGCTCCGGCGAGGGGTCT |
| | | ACAAAGGGAGATATACAGATGACACAGTCCCCCAGTTC |
| | | CCTGTCCGCCTCAGTGGGAGACCGAGTGACGATTACCTG |
| | | TCGTGCCAGCCAGGACGTCAATACCGCCGTCGCTTGGTA |
| | | TCAGCAAAAACCAGGCAAGGCCCCGAAACTATTGATCT |
| | | ACAGTGCCTCTTTTCTGTACTCCGGGGTGCCGAGCAGAT |
| | | TTAGTGGCTCCAGGAGCGGAACCGATTTCACCCTAACCA |
| | | TTTCCAGTTTGCAGCCAGAGGATTTCGCGACCTATTACT |
| | | GCCAGCAACACTACACCACACCGCCAACTTTCGGACAA |
| | | GGAACCAAGGTTGAAATCAAAACTACGACCCCAGCACC |
| | | TAGACCTCCCACCCCAGCTCCAACTATAGCTTCCCAGCC |
| | | ATTGTCTCTCCGGCCAGAGGCGTGTCGACCAGCCGCTGG |
| | | AGGGGCCGTTCATACAAGAGGACTCGATTTCGCTTGCGA |
| | | TATCTACATATGGGCCCCTCTTGCCGGGACATGCGGTGT |
| | | CCTGCTTCTAAGCTTGGTTATTACCCTCTATTGCAAACGC |
| | | GGCCGCAAGAAACTGCTCTACATCTTTAAACAGCCGTTC |
| | | ATGAGGCCTGTGCAGACAACGCAGGAAGAGGATGGCTG |
| | | TAGTTGTCGGTTTCCGGAAGAGGAAGAGGGGGGCTGCG |
| | | AGTTGCGTGTCAAATTTTCTCGGTCTGCCGACGCCCCCG |
| | | CGTACCAGCAAGGGCAGAACCAGCTTTATAATGAGCTG |
| | | AATCTTGGACGACGGGAGGAATATGACGTGCTTGACAA |
| | | GAGGCGAGGTAGGGACCCTGAGATGGGGGGAAAACCTC |
| | | GGAGGAAAAACCCACAGGAAGGCCTGTATAACGAACTG |
| | | CAGAAGGACAAGATGGCTGAAGCCTACTCTGAGATTGG |
| | | AATGAAAGGGGAACGCAGACGCGGCAAGGGCCATGATG |
| | | GCCTCTACCAAGGTCTAAGCACTGCCACCAAGGACACCT |
| | | ATGACGCACTCCACATGCAAGCTCTACCTCCCCGTTGAT |
| | | AA |
| MC0290- | SEQ ID | MALPVTALLLPLALLLHAARPQVQLVQSGGGVVQPGGSLR |
| 3PF12_28_ | NO: 22 | VSCAASGVTLSDYGMHWVRQAPGKGLEWMAFIRNDGSD |
| LIR1_HER | | KYYADSVKGRFTISRDNSKKTVSLQMSSLRAEDTAVYYCA |
| 2 Protein | | KNGESGPLDYWYFDLWGRGTLVTVSSGGGGSGGGGSGGG |
| sequence | | GSDIVMTQSPSFLSASVGDRVTITCRASHGINNYLAWYQQ |
| (VR290) | | KPGKAPKLLIYAASTLQSGVPSRFSGSGSGTEFTLTISSLQPE |
| | | DFATYYCQQYDSYPPTFGRGTKVEIKIEVMYPPPYLDNEKS |
| | | NGTIIHVKGKHLCPSPLFPGPSKPFWVLVVVGGVLACYSLL |
| | | VTVAFIIFWVLRHRRQGKHWTSTQRKADFQHPAGAVGPEP |
| | | TDRGLQWRSSPAADAQEENLYAAVKHTQPEDGVEMDTRS |
| | | PHDEDPQAVTYAEVKHSRPRREMASPPSPLSGEFLDTKDR |
| | | QAEEDRQMDTEAAASEAPQDVTYAQLHSLTLRREATEPPP |
| | | SQEGPSPAVPSIYATLAIHRRKRGSGEGRGSLLTCGDVEEN |
| | | PGPMALPVTALLLPLALLLHAARPEVQLVESGGGLVQPGG |
| | | SLRLSCAASGFNIKDTYIHWVRQAPGKGLEWVARIYPTNG |
| | | YTRYADSVKGRFTISADTSKNTAYLQMNSLRAEDTAVYYC |
| | | SRWGGDGFYAMDYWGQGTLVTVSSGSTSGSGKPGSGEGS |
| | | TKGDIQMTQSPSSLSASVGDRVTITCRASQDVNTAVAWYQ |
| | | QKPGKAPKLLIYSASFLYSGVPSRFSGSRSGTDFTLTISSLQP |
| | | EDFATYYCQQHYTTPPTFGQGTKVEIKTTTPAPRPPTPAPTI |
| | | ASQPLSLRPEACRPAAGGAVHTRGLDFACDIYIWAPLAGT |
| | | CGVLLLSLVITLYCKRGRKKLLYIFKQPFMRPVQTTQEEDG |
| | | CSCRFPEEEEGGCELRVKFSRSADAPAYQQGQNQLYNELN |
| | | LGRREEYDVLDKRRGRDPEMGGKPRRKNPQEGLYNELQK |
| | | DKMAEAYSEIGMKGERRRGKGHDGLYQGLSTATKDTYDA |
| | | LHMQALPPR |
| MC0291- | SEQ ID | ATGGCACTGCCAGTGACCGCCCTGCTGCTGCCTCTGGCC |
| 3PF12_28_ | NO: 23 | CTGCTGCTGCACGCAGCCAGACCCCAAGTGCAACTAGTC |
| KIR2DL1_ | | CAATCAGGTGGAGGCGTCGTGCAACCTGGAGGGTCCCT |
| HER2 | | CCGCGTTAGCTGCGCCGCATCAGGCGTTACCTTGTCAGA |
| nucleotide | | CTACGGCATGCATTGGGTTAGGCAAGCCCCCGGCAAGG |
| Sequence | | GGCTCGAATGGATGGCTTTCATTCGGAATGACGGGAGC |
| (VR291) | | GATAAATATTACGCGGATTCAGTTAAAGGGCGGTTCACC |
| | | ATCAGCCGCGACAATAGCAAAAAGACGGTCTCCTTACA |
| | | GATGTCCAGCTTGCGGGCCGAAGACACGGCTGTATACTA |
| | | TTGTGCTAAAAATGGCGAGAGCGGCCCCCTGGATTACTG |
| | | GTACTTTGACCTGTGGGGCAGAGGCACCCTGGTCACGGT |
| | | GTCCTCTGGAGGAGGAGGCTCCGGAGGAGGAGGCTCTG |
| | | GCGGCGGCGGCAGCGACATTGTAATGACCCAGTCACCC |
| | | TCCTTCCTTAGTGCCTCAGTCGGAGACCGCGTGACTATC |
| | | ACTTGTCGTGCCTCACACGGAATTAATAACTACCTCGCT |
| | | TGGTATCAGCAAAAACCAGGCAAGGCCCCGAAACTATT |
| | | GATCTACGCCGCATCTACTCTGCAGAGCGGAGTACCGAG |
| | | CAGATTTAGTGGTTCCGGCAGCGGAACCGAGTTCACCCT |
| | | AACCATTTCCAGTTTGCAGCCAGAGGATTTCGCGACCTA |
| | | TTACTGCCAGCAATACGATTCATACCCGCCAACTTTCGG |
| | | AAGAGGTACCAAGGTTGAAATCAAGATTGAAGTTATGT |
| | | ATCCTCCTCCTTACCTAGACAATGAGAAGAGCAATGGAA |
| | | CCATTATCCATGTGAAAGGGAAACACCTTTGTCCAAGTC |
| | | CCCTATTTCCCGGGCCTTCGAAGCCCTTTTGGGTGCTGGT |
| | | GGTGGTTGGTGGAGTCCTGGCTTGCTATAGCTTGCTAGT |
| | | AACAGTAGCGTTTATTATTTTCTGGGTGCATAGGTGGTG |
| | | CTCAAACAAAAAGAATGCTGCCGTCATGGACCAGGAGA |
| | | GCGCGGGCAATCGGACCGCAAACTCAGAGGACTCAGAT |
| | | GAACAAGATCCACAGGAAGTGACCTACACTCAGCTGAA |
| | | CCATTGTGTGTTTACACAGCGCAAGATTACTCGTCCAAG |
| | | CCAGCGTCCTAAGACCCCCCCGACCGATATCATTGTGTA |
| | | TACCGAGCTTCCTAATGCCGAATCCCGCAGCAAGGTGGT |
| | | CTCCTGCCCGCGGAGAAAGCGTGGATCCGGGGAAGGCC |
| | | GAGGCTCCCTTCTAACATGTGGAGATGTCGAGGAAAAC |
| | | CCTGGCCCTATGGCGCTGCCAGTCACTGCATTGTTATTG |
| | | CCTCTGGCCCTGCTTCTCCATGCGGCGCGCCCAGAAGTG |
| | | CAGCTGGTCGAGAGCGGAGGCGGACTGGTTCAACCCGG |
| | | AGGCAGCTTGAGACTGTCCTGCGCGGCCAGCGGCTTCAA |
| | | CATCAAGGATACCTATATCCACTGGGTGAGGCAGGCTCC |
| | | AGGAAAGGGCCTGGAGTGGGTGGCAAGGATTTACCCTA |
| | | CTAATGGATATACACGCTACGCTGATTCCGTGAAGGGAC |
| | | GCTTTACAATCTCAGCAGATACATCCAAAAACACGGCCT |
| | | ATTTACAGATGAATAGTTTGCGGGCCGAAGACACGGCT |
| | | GTATACTATTGTTCTCGGTGGGGGGGCGATGGATTTTAT |
| | | GCGATGGATTACTGGGGCCAGGGCACCCTGGTAACCGT |
| | | GTCAAGCGGCTCAACATCCGGGTCCGGTAAGCCGGGCT |
| | | CCGGCGAGGGGTCTACAAAGGGAGATATACAGATGACA |
| | | CAGTCCCCCAGTTCCCTGTCCGCCTCAGTGGGAGACCGA |
| | | GTGACGATTACCTGTCGTGCCAGCCAGGACGTCAATACC |
| | | GCCGTCGCTTGGTATCAGCAAAAACCAGGCAAGGCCCC |
| | | GAAACTATTGATCTACAGTGCCTCTTTTCTGTACTCCGG |
| | | GGTGCCGAGCAGATTTAGTGGCTCCAGGAGCGGAACCG |
| | | ATTTCACCCTAACCATTTCCAGTTTGCAGCCAGAGGATT |
| | | TCGCGACCTATTACTGCCAGCAACACTACACCACACCGC |
| | | CAACTTTCGGACAAGGAACCAAGGTTGAAATCAAAACT |
| | | ACGACCCCAGCACCTAGACCTCCCACCCCAGCTCCAACT |
| | | ATAGCTTCCCAGCCATTGTCTCTCCGGCCAGAGGCGTGT |
| | | CGACCAGCCGCTGGAGGGGCCGTTCATACAAGAGGACT |
| | | CGATTTCGCTTGCGATATCTACATATGGGCCCCTCTTGCC |
| | | GGGACATGCGGTGTCCTGCTTCTAAGCTTGGTTATTACC |
| | | CTCTATTGCAAACGCGGCCGCAAGAAACTGCTCTACATC |
| | | TTTAAACAGCCGTTCATGAGGCCTGTGCAGACAACGCAG |
| | | GAAGAGGATGGCTGTAGTTGTCGGTTTCCGGAAGAGGA |
| | | AGAGGGGGGCTGCGAGTTGCGTGTCAAATTTTCTCGGTC |
| | | TGCCGACGCCCCCGCGTACCAGCAAGGGCAGAACCAGC |
| | | TTTATAATGAGCTGAATCTTGGACGACGGGAGGAATATG |
| | | ACGTGCTTGACAAGAGGCGAGGTAGGGACCCTGAGATG |
| | | GGGGGAAAACCTCGGAGGAAAAACCCACAGGAAGGCCT |
| | | GTATAACGAACTGCAGAAGGACAAGATGGCTGAAGCCT |
| | | ACTCTGAGATTGGAATGAAAGGGGAACGCAGACGCGGC |
| | | AAGGGCCATGATGGCCTCTACCAAGGTCTAAGCACTGCC |
| | | ACCAAGGACACCTATGACGCACTCCACATGCAAGCTCTA |
| | | CCTCCCCGTTGATAA |
| MC0291- | SEQ ID | MALPVTALLLPLALLLHAARPQVQLVQSGGGVVQPGGSLR |
| 3PF12_28_ | NO: 24 | VSCAASGVTLSDYGMHWVRQAPGKGLEWMAFIRNDGSD |
| KIR2DL1_ | | KYYADSVKGRFTISRDNSKKTVSLQMSSLRAEDTAVYYCA |
| HER2 | | KNGESGPLDYWYFDLWGRGTLVTVSSGGGGSGGGGSGGG |
| Protein | | GSDIVMTQSPSFLSASVGDRVTITCRASHGINNYLAWYQQ |
| Sequence | | KPGKAPKLLIYAASTLQSGVPSRFSGSGSGTEFTLTISSLQPE |
| (VR291) | | DFATYYCQQYDSYPPTFGRGTKVEIKIEVMYPPPYLDNEKS |
| | | NGTIIHVKGKHLCPSPLFPGPSKPFWVLVVVGGVLACYSLL |
| | | VTVAFIIFWVHRWCSNKKNAAVMDQESAGNRTANSEDSD |
| | | EQDPQEVTYTQLNHCVFTQRKITRPSQRPKTPPTDIIVYTEL |
| | | PNAESRSKVVSCPRRKRGSGEGRGSLLTCGDVEENPGPMA |
| | | LPVTALLLPLALLLHAARPEVQLVESGGGLVQPGGSLRLSC |
| | | AASGFNIKDTYIHWVRQAPGKGLEWVARIYPTNGYTRYA |
| | | DSVKGRFTISADTSKNTAYLQMNSLRAEDTAVYYCSRWG |
| | | GDGFYAMDYWGQGTLVTVSSGSTSGSGKPGSGEGSTKGDI |
| | | QMTQSPSSLSASVGDRVTITCRASQDVNTAVAWYQQKPG |
| | | KAPKLLIYSASFLYSGVPSRFSGSRSGTDFTLTISSLQPEDFA |
| | | TYYCQQHYTTPPTFGQGTKVEIKTTTPAPRPPTPAPTIASQP |
| | | LSLRPEACRPAAGGAVHTRGLDFACDIYIWAPLAGTCGVL |
| | | LLSLVITLYCKRGRKKLLYIFKQPFMRPVQTTQEEDGCSCR |
| | | FPEEEEGGCELRVKFSRSADAPAYQQGQNQLYNELNLGRR |
| | | EEYDVLDKRRGRDPEMGGKPRRKNPQEGLYNELQKDKM |
| | | AEAYSEIGMKGERRRGKGHDGLYQGLSTATKDTYDALHM |
| | | QALPPR |
| MC0292- | SEQ ID | ATGGCACTGCCAGTGACCGCCCTGCTGCTGCCTCTGGCC |
| BB7.2_28_ | NO: 25 | CTGCTGCTGCACGCAGCCAGACCCCAGGTGCAGCTGCA |
| LIR1_HER2 | | GCAGTCTGGACCTGAGCTGGTGAAGCCAGGAGCCTCCG |
| nucleotide | | TGAAGATGTCTTGCAAGGCCAGCGGCTACACCTTCACAT |
| Sequence | | CTTATCACATCCAGTGGGTGAAGCAGCGGCCCGGACAG |
| (VR292) | | GGCCTGGAGTGGATCGGATGGATCTACCCAGGCGACGG |
| | | CTCCACACAGTATAACGAGAAGTTCAAGGGCAAGACCA |
| | | CACTGACCGCCGATAAGAGCAGCAGCACCGCCTACATG |
| | | CTGCTGAGCAGCCTGACCAGCGAGGACAGCGCCATCTA |
| | | CTTTTGCGCCAGGGAGGGCACATACTATGCTATGGACTA |
| | | TTGGGGCCAGGGCACCAGCGTGACAGTGTCTAGCGGAG |
| | | GAGGAGGCTCCGGAGGAGGAGGCTCTGGCGGCGGCGGC |
| | | AGCGACGTGCTGATGACCCAGACACCACTGAGCCTGCC |
| | | CGTGAGCCTGGGCGATCAGGTGAGCATCTCCTGTAGATC |
| | | CTCTCAGAGCATCGTGCACTCCAACGGCAATACCTACCT |
| | | GGAGTGGTATCTGCAGAAGCCAGGCCAGTCCCCCAAGC |
| | | TGCTGATCTATAAGGTGTCTAATCGGTTCAGCGGCGTGC |
| | | CTGACAGATTTTCTGGCAGCGGCTCCGGCACCGACTTCA |
| | | CCCTGAAGATCAGCCGGGTGGAGGCAGAGGATCTGGGC |
| | | GTGTACTATTGTTTCCAGGGCTCCCACGTGCCACGCACC |
| | | TTTGGCGGCGGTACCAAGCTGGAGATCAAGATTGAAGTT |
| | | ATGTATCCTCCTCCTTACCTAGACAATGAGAAGAGCAAT |
| | | GGAACCATTATCCATGTGAAAGGGAAACACCTTTGTCCA |
| | | AGTCCCCTATTTCCCGGGCCTTCGAAGCCCTTTTGGGTG |
| | | CTGGTGGTGGTTGGTGGAGTCCTGGCTTGCTATAGCTTG |
| | | CTAGTAACAGTAGCGTTTATTATTTTCTGGGTGCTGCGC |
| | | CACAGGAGACAGGGCAAGCACTGGACCAGCACCCAGCG |
| | | GAAGGCCGACTTTCAGCACCCTGCCGGCGCCGTGGGCCC |
| | | TGAGCCTACCGACAGGGGCCTGCAGTGGAGGAGCTCCC |
| | | CAGCCGCCGATGCCCAGGAGGAGAATCTGTACGCCGCC |
| | | GTGAAGCACACCCAGCCAGAGGACGGCGTGGAGATGGA |
| | | CACCCGCTCCCCACACGACGAGGATCCACAGGCCGTGA |
| | | CCTACGCCGAGGTGAAGCACAGCCGCCCCAGACGCGAG |
| | | ATGGCCAGCCCACCCAGCCCCCTGTCCGGCGAGTTCCTG |
| | | GACACCAAGGACAGGCAGGCCGAGGAGGACCGGCAGA |
| | | TGGACACCGAGGCCGCCGCCTCCGAGGCCCCCCAGGAC |
| | | GTGACCTACGCCCAGCTGCACTCCCTGACCCTGCGGAGA |
| | | GAGGCCACCGAGCCCCCACCCAGCCAGGAGGGCCCCTC |
| | | CCCCGCCGTGCCTAGCATCTACGCCACCCTGGCCATCCA |
| | | CCGGAGAAAGCGTGGATCCGGGGAAGGCCGAGGCTCCC |
| | | TTCTAACATGTGGAGATGTCGAGGAAAACCCTGGCCCTA |
| | | TGGCGCTGCCAGTCACTGCATTGTTATTGCCTCTGGCCCT |
| | | GCTTCTCCATGCGGCGCGCCCAGAAGTGCAGCTGGTCGA |
| | | GAGCGGAGGCGGACTGGTTCAACCCGGAGGCAGCTTGA |
| | | GACTGTCCTGCGCGGCCAGCGGCTTCAACATCAAGGATA |
| | | CCTATATCCACTGGGTGAGGCAGGCTCCAGGAAAGGGC |
| | | CTGGAGTGGGTGGCAAGGATTTACCCTACTAATGGATAT |
| | | ACACGCTACGCTGATTCCGTGAAGGGACGCTTTACAATC |
| | | TCAGCAGATACATCCAAAAACACGGCCTATTTACAGATG |
| | | AATAGTTTGCGGGCCGAAGACACGGCTGTATACTATTGT |
| | | TCTCGGTGGGGGGGCGATGGATTTTATGCGATGGATTAC |
| | | TGGGGCCAGGGCACCCTGGTAACCGTGTCAAGCGGCTC |
| | | AACATCCGGGTCCGGTAAGCCGGGCTCCGGCGAGGGGT |
| | | CTACAAAGGGAGATATACAGATGACACAGTCCCCCAGT |
| | | TCCCTGTCCGCCTCAGTGGGAGACCGAGTGACGATTACC |
| | | TGTCGTGCCAGCCAGGACGTCAATACCGCCGTCGCTTGG |
| | | TATCAGCAAAAACCAGGCAAGGCCCCGAAACTATTGAT |
| | | CTACAGTGCCTCTTTTCTGTACTCCGGGGTGCCGAGCAG |
| | | ATTTAGTGGCTCCAGGAGCGGAACCGATTTCACCCTAAC |
| | | CATTTCCAGTTTGCAGCCAGAGGATTTCGCGACCTATTA |
| | | CTGCCAGCAACACTACACCACACCGCCAACTTTCGGACA |
| | | AGGAACCAAGGTTGAAATCAAAACTACGACCCCAGCAC |
| | | CTAGACCTCCCACCCCAGCTCCAACTATAGCTTCCCAGC |
| | | CATTGTCTCTCCGGCCAGAGGCGTGTCGACCAGCCGCTG |
| | | GAGGGGCCGTTCATACAAGAGGACTCGATTTCGCTTGCG |
| | | ATATCTACATATGGGCCCCTCTTGCCGGGACATGCGGTG |
| | | TCCTGCTTCTAAGCTTGGTTATTACCCTCTATTGCAAACG |
| | | CGGCCGCAAGAAACTGCTCTACATCTTTAAACAGCCGTT |
| | | CATGAGGCCTGTGCAGACAACGCAGGAAGAGGATGGCT |
| | | GTAGTTGTCGGTTTCCGGAAGAGGAAGAGGGGGGCTGC |
| | | GAGTTGCGTGTCAAATTTTCTCGGTCTGCCGACGCCCCC |
| | | GCGTACCAGCAAGGGCAGAACCAGCTTTATAATGAGCT |
| | | GAATCTTGGACGACGGGAGGAATATGACGTGCTTGACA |
| | | AGAGGCGAGGTAGGGACCCTGAGATGGGGGGAAAACCT |
| | | CGGAGGAAAAACCCACAGGAAGGCCTGTATAACGAACT |
| | | GCAGAAGGACAAGATGGCTGAAGCCTACTCTGAGATTG |
| | | GAATGAAAGGGGAACGCAGACGCGGCAAGGGCCATGAT |
| | | GGCCTCTACCAAGGTCTAAGCACTGCCACCAAGGACAC |
| | | CTATGACGCACTCCACATGCAAGCTCTACCTCCCCGTTG |
| | | ATAA |
| BB7.2_28_ | SEQ ID | MALPVTALLLPLALLLHAARPQVQLQQSGPELVKPGASVK |
| LIR1_HER | NO: 26 | MSCKASGYTFTSYHIQWVKQRPGQGLEWIGWIYPGDGSTQ |
| 2 Protein | | YNEKFKGKTTLTADKSSSTAYMLLSSLTSEDSAIYFCAREG |
| Sequence | | TYYAMDYWGQGTSVTVSSGGGGSGGGGSGGGGSDVLMT |
| (VR292) | | QTPLSLPVSLGDQVSISCRSSQSIVHSNGNTYLEWYLQKPG |
| | | QSPKLLIYKVSNRFSGVPDRFSGSGSGTDFTLKISRVEAEDL |
| | | GVYYCFQGSHVPRTFGGGTKLEIKIEVMYPPPYLDNEKSN |
| | | GTIIHVKGKHLCPSPLFPGPSKPFWVLVVVGGVLACYSLLV |
| | | TVAFIIFWVLRHRRQGKHWTSTQRKADFQHPAGAVGPEPT |
| | | DRGLQWRSSPAADAQEENLYAAVKHTQPEDGVEMDTRSP |
| | | HDEDPQAVTYAEVKHSRPRREMASPPSPLSGEFLDTKDRQ |
| | | AEEDRQMDTEAAASEAPQDVTYAQLHSLTLRREATEPPPS |
| | | QEGPSPAVPSIYATLAIHRRKRGSGEGRGSLLTCGDVEENP |
| | | GPMALPVTALLLPLALLLHAARPEVQLVESGGGLVQPGGS |
| | | LRLSCAASGFNIKDTYIHWVRQAPGKGLEWVARIYPTNGY |
| | | TRYADSVKGRFTISADTSKNTAYLQMNSLRAEDTAVYYCS |
| | | RWGGDGFYAMDYWGQGTLVTVSSGSTSGSGKPGSGEGST |
| | | KGDIQMTQSPSSLSASVGDRVTITCRASQDVNTAVAWYQQ |
| | | KPGKAPKLLIYSASFLYSGVPSRFSGSRSGTDFTLTISSLQPE |
| | | DFATYYCQQHYTTPPTFGQGTKVEIKTTTPAPRPPTPAPTIA |
| | | SQPLSLRPEACRPAAGGAVHTRGLDFACDIYIWAPLAGTC |
| | | GVLLLSLVITLYCKRGRKKLLYIFKQPFMRPVQTTQEEDGC |
| | | SCRFPEEEEGGCELRVKFSRSADAPAYQQGQNQLYNELNL |
| | | GRREEYDVLDKRRGRDPEMGGKPRRKNPQEGLYNELQKD |
| | | KMAEAYSEIGMKGERRRGKGHDGLYQGLSTATKDTYDAL |
| | | HMQALPPR |
| MC0293- | SEQ ID | ATGGCACTGCCAGTGACCGCCCTGCTGCTGCCTCTGGCC |
| BB7.2_28_ | NO: 27 | CTGCTGCTGCACGCAGCCAGACCCCAGGTGCAGCTGCA |
| KIR2DL1_ | | GCAGTCTGGACCTGAGCTGGTGAAGCCAGGAGCCTCCG |
| HER2 | | TGAAGATGTCTTGCAAGGCCAGCGGCTACACCTTCACAT |
| nucleotide | | CTTATCACATCCAGTGGGTGAAGCAGCGGCCCGGACAG |
| sequence | | GGCCTGGAGTGGATCGGATGGATCTACCCAGGCGACGG |
| (VR293) | | CTCCACACAGTATAACGAGAAGTTCAAGGGCAAGACCA |
| | | CACTGACCGCCGATAAGAGCAGCAGCACCGCCTACATG |
| | | CTGCTGAGCAGCCTGACCAGCGAGGACAGCGCCATCTA |
| | | CTTTTGCGCCAGGGAGGGCACATACTATGCTATGGACTA |
| | | TTGGGGCCAGGGCACCAGCGTGACAGTGTCTAGCGGAG |
| | | GAGGAGGCTCCGGAGGAGGAGGCTCTGGCGGCGGCGGC |
| | | AGCGACGTGCTGATGACCCAGACACCACTGAGCCTGCC |
| | | CGTGAGCCTGGGCGATCAGGTGAGCATCTCCTGTAGATC |
| | | CTCTCAGAGCATCGTGCACTCCAACGGCAATACCTACCT |
| | | GGAGTGGTATCTGCAGAAGCCAGGCCAGTCCCCCAAGC |
| | | TGCTGATCTATAAGGTGTCTAATCGGTTCAGCGGCGTGC |
| | | CTGACAGATTTTCTGGCAGCGGCTCCGGCACCGACTTCA |
| | | CCCTGAAGATCAGCCGGGTGGAGGCAGAGGATCTGGGC |
| | | GTGTACTATTGTTTCCAGGGCTCCCACGTGCCACGCACC |
| | | TTTGGCGGCGGTACCAAGCTGGAGATCAAGATTGAAGTT |
| | | ATGTATCCTCCTCCTTACCTAGACAATGAGAAGAGCAAT |
| | | GGAACCATTATCCATGTGAAAGGGAAACACCTTTGTCCA |
| | | AGTCCCCTATTTCCCGGGCCTTCGAAGCCCTTTTGGGTG |
| | | CTGGTGGTGGTTGGTGGAGTCCTGGCTTGCTATAGCTTG |
| | | CTAGTAACAGTAGCGTTTATTATTTTCTGGGTGCATAGG |
| | | TGGTGCTCAAACAAAAAGAATGCTGCCGTCATGGACCA |
| | | GGAGAGCGCGGGCAATCGGACCGCAAACTCAGAGGACT |
| | | CAGATGAACAAGATCCACAGGAAGTGACCTACACTCAG |
| | | CTGAACCATTGTGTGTTTACACAGCGCAAGATTACTCGT |
| | | CCAAGCCAGCGTCCTAAGACCCCCCCGACCGATATCATT |
| | | GTGTATACCGAGCTTCCTAATGCCGAATCCCGCAGCAAG |
| | | GTGGTCTCCTGCCCGCGGAGAAAGCGTGGATCCGGGGA |
| | | AGGCCGAGGCTCCCTTCTAACATGTGGAGATGTCGAGG |
| | | AAAACCCTGGCCCTATGGCGCTGCCAGTCACTGCATTGT |
| | | TATTGCCTCTGGCCCTGCTTCTCCATGCGGCGCGCCCAG |
| | | AAGTGCAGCTGGTCGAGAGCGGAGGCGGACTGGTTCAA |
| | | CCCGGAGGCAGCTTGAGACTGTCCTGCGCGGCCAGCGG |
| | | CTTCAACATCAAGGATACCTATATCCACTGGGTGAGGCA |
| | | GGCTCCAGGAAAGGGCCTGGAGTGGGTGGCAAGGATTT |
| | | ACCCTACTAATGGATATACACGCTACGCTGATTCCGTGA |
| | | AGGGACGCTTTACAATCTCAGCAGATACATCCAAAAAC |
| | | ACGGCCTATTTACAGATGAATAGTTTGCGGGCCGAAGAC |
| | | ACGGCTGTATACTATTGTTCTCGGTGGGGGGGCGATGGA |
| | | TTTTATGCGATGGATTACTGGGGCCAGGGCACCCTGGTA |
| | | ACCGTGTCAAGCGGCTCAACATCCGGGTCCGGTAAGCC |
| | | GGGCTCCGGCGAGGGGTCTACAAAGGGAGATATACAGA |
| | | TGACACAGTCCCCCAGTTCCCTGTCCGCCTCAGTGGGAG |
| | | ACCGAGTGACGATTACCTGTCGTGCCAGCCAGGACGTCA |
| | | ATACCGCCGTCGCTTGGTATCAGCAAAAACCAGGCAAG |
| | | GCCCCGAAACTATTGATCTACAGTGCCTCTTTTCTGTACT |
| | | CCGGGGTGCCGAGCAGATTTAGTGGCTCCAGGAGCGGA |
| | | ACCGATTTCACCCTAACCATTTCCAGTTTGCAGCCAGAG |
| | | GATTTCGCGACCTATTACTGCCAGCAACACTACACCACA |
| | | CCGCCAACTTTCGGACAAGGAACCAAGGTTGAAATCAA |
| | | AACTACGACCCCAGCACCTAGACCTCCCACCCCAGCTCC |
| | | AACTATAGCTTCCCAGCCATTGTCTCTCCGGCCAGAGGC |
| | | GTGTCGACCAGCCGCTGGAGGGGCCGTTCATACAAGAG |
| | | GACTCGATTTCGCTTGCGATATCTACATATGGGCCCCTC |
| | | TTGCCGGGACATGCGGTGTCCTGCTTCTAAGCTTGGTTA |
| | | TTACCCTCTATTGCAAACGCGGCCGCAAGAAACTGCTCT |
| | | ACATCTTTAAACAGCCGTTCATGAGGCCTGTGCAGACAA |
| | | CGCAGGAAGAGGATGGCTGTAGTTGTCGGTTTCCGGAA |
| | | GAGGAAGAGGGGGGCTGCGAGTTGCGTGTCAAATTTTC |
| | | TCGGTCTGCCGACGCCCCCGCGTACCAGCAAGGGCAGA |
| | | ACCAGCTTTATAATGAGCTGAATCTTGGACGACGGGAG |
| | | GAATATGACGTGCTTGACAAGAGGCGAGGTAGGGACCC |
| | | TGAGATGGGGGGAAAACCTCGGAGGAAAAACCCACAGG |
| | | AAGGCCTGTATAACGAACTGCAGAAGGACAAGATGGCT |
| | | GAAGCCTACTCTGAGATTGGAATGAAAGGGGAACGCAG |
| | | ACGCGGCAAGGGCCATGATGGCCTCTACCAAGGTCTAA |
| | | GCACTGCCACCAAGGACACCTATGACGCACTCCACATGC |
| | | AAGCTCTACCTCCCCGTTGATAA |
| MC0293- | SEQ ID | MALPVTALLLPLALLLHAARPQVQLQQSGPELVKPGASVK |
| BB7.2_28_ | NO: 28 | MSCKASGYTFTSYHIQWVKQRPGQGLEWIGWIYPGDGSTQ |
| KIR2DL1_ | | YNEKFKGKTTLTADKSSSTAYMLLSSLTSEDSAIYFCAREG |
| HER2 | | TYYAMDYWGQGTSVTVSSGGGGSGGGGSGGGGSDVLMT |
| Protein | | QTPLSLPVSLGDQVSISCRSSQSIVHSNGNTYLEWYLQKPG |
| Sequence | | QSPKLLIYKVSNRFSGVPDRFSGSGSGTDFTLKISRVEAEDL |
| (VR293) | | GVYYCFQGSHVPRTFGGGTKLEIKIEVMYPPPYLDNEKSN |
| | | GTIIHVKGKHLCPSPLFPGPSKPFWVLVVVGGVLACYSLLV |
| | | TVAFIIFWVHRWCSNKKNAAVMDQESAGNRTANSEDSDE |
| | | QDPQEVTYTQLNHCVFTQRKITRPSQRPKTPPTDIIVYTELP |
| | | NAESRSKVVSCPRRKRGSGEGRGSLLTCGDVEENPGPMAL |
| | | PVTALLLPLALLLHAARPEVQLVESGGGLVQPGGSLRLSCA |
| | | ASGFNIKDTYIHWVRQAPGKGLEWVARIYPTNGYTRYADS |
| | | VKGRFTISADTSKNTAYLQMNSLRAEDTAVYYCSRWGGD |
| | | GFYAMDYWGQGTLVTVSSGSTSGSGKPGSGEGSTKGDIQ |
| | | MTQSPSSLSASVGDRVTITCRASQDVNTAVAWYQQKPGK |
| | | APKLLIYSASFLYSGVPSRFSGSRSGTDFTLTISSLQPEDFAT |
| | | YYCQQHYTTPPTFGQGTKVEIKTTTPAPRPPTPAPTIASQPL |
| | | SLRPEACRPAAGGAVHTRGLDFACDIYIWAPLAGTCGVLL |
| | | LSLVITLYCKRGRKKLLYIFKQPFMRPVQTTQEEDGCSCRF |
| | | PEEEEGGCELRVKFSRSADAPAYQQGQNQLYNELNLGRRE |
| | | EYDVLDKRRGRDPEMGGKPRRKNPQEGLYNELQKDKMA |
| | | EAYSEIGMKGERRRGKGHDGLYQGLSTATKDTYDALHMQ |
| | | ALPPR |
| MC0294- | SEQ ID | ATGGCACTGCCAGTGACCGCCCTGCTGCTGCCTCTGGCC |
| 3PF12_CD8_ | NO: 29 | CTGCTGCTGCACGCAGCCAGACCCCAAGTGCAACTAGTC |
| _LIR1_HE | | CAATCAGGTGGAGGCGTCGTGCAACCTGGAGGGTCCCT |
| R2 | | CCGCGTTAGCTGCGCCGCATCAGGCGTTACCTTGTCAGA |
| Nucleotide | | CTACGGCATGCATTGGGTTAGGCAAGCCCCCGGCAAGG |
| sequence | | GGCTCGAATGGATGGCTTTCATTCGGAATGACGGGAGC |
| (VR294) | | GATAAATATTACGCGGATTCAGTTAAAGGGCGGTTCACC |
| | | ATCAGCCGCGACAATAGCAAAAAGACGGTCTCCTTACA |
| | | GATGTCCAGCTTGCGGGCCGAAGACACGGCTGTATACTA |
| | | TTGTGCTAAAAATGGCGAGAGCGGCCCCCTGGATTACTG |
| | | GTACTTTGACCTGTGGGGCAGAGGCACCCTGGTCACGGT |
| | | GTCCTCTGGAGGAGGAGGCTCCGGAGGAGGAGGCTCTG |
| | | GCGGCGGCGGCAGCGACATTGTAATGACCCAGTCACCC |
| | | TCCTTCCTTAGTGCCTCAGTCGGAGACCGCGTGACTATC |
| | | ACTTGTCGTGCCTCACACGGAATTAATAACTACCTCGCT |
| | | TGGTATCAGCAAAAACCAGGCAAGGCCCCGAAACTATT |
| | | GATCTACGCCGCATCTACTCTGCAGAGCGGAGTACCGAG |
| | | CAGATTTAGTGGTTCCGGCAGCGGAACCGAGTTCACCCT |
| | | AACCATTTCCAGTTTGCAGCCAGAGGATTTCGCGACCTA |
| | | TTACTGCCAGCAATACGATTCATACCCGCCAACTTTCGG |
| | | AAGAGGTACCAAGGTTGAAATCAAGACTACGACCCCAG |
| | | CACCTAGACCTCCCACCCCAGCTCCAACTATAGCTTCCC |
| | | AGCCATTGTCTCTCCGGCCAGAGGCGTGTCGACCAGCCG |
| | | CTGGAGGGGCCGTTCATACAAGAGGACTCGATTTCGCTT |
| | | GCGATATCTACATATGGGCCCCTCTTGCCGGGACATGCG |
| | | GTGTCCTGCTTCTAAGCTTGGTTATTACCCTCTATTGCCT |
| | | GCGCCACAGGAGACAGGGCAAGCACTGGACCAGCACCC |
| | | AGCGGAAGGCCGACTTTCAGCACCCTGCCGGCGCCGTG |
| | | GGCCCTGAGCCTACCGACAGGGGCCTGCAGTGGAGGAG |
| | | CTCCCCAGCCGCCGATGCCCAGGAGGAGAATCTGTACG |
| | | CCGCCGTGAAGCACACCCAGCCAGAGGACGGCGTGGAG |
| | | ATGGACACCCGCTCCCCACACGACGAGGATCCACAGGC |
| | | CGTGACCTACGCCGAGGTGAAGCACAGCCGCCCCAGAC |
| | | GCGAGATGGCCAGCCCACCCAGCCCCCTGTCCGGCGAG |
| | | TTCCTGGACACCAAGGACAGGCAGGCCGAGGAGGACCG |
| | | GCAGATGGACACCGAGGCCGCCGCCTCCGAGGCCCCCC |
| | | AGGACGTGACCTACGCCCAGCTGCACTCCCTGACCCTGC |
| | | GGAGAGAGGCCACCGAGCCCCCACCCAGCCAGGAGGGC |
| | | CCCTCCCCCGCCGTGCCTAGCATCTACGCCACCCTGGCC |
| | | ATCCACCGGAGAAAGCGTGGATCCGGGGAAGGCCGAGG |
| | | CTCCCTTCTAACATGTGGAGATGTCGAGGAAAACCCTGG |
| | | CCCTATGGCGCTGCCAGTCACTGCATTGTTATTGCCTCTG |
| | | GCCCTGCTTCTCCATGCGGCGCGCCCAGAAGTGCAGCTG |
| | | GTCGAGAGCGGAGGCGGACTGGTTCAACCCGGAGGCAG |
| | | CTTGAGACTGTCCTGCGCGGCCAGCGGCTTCAACATCAA |
| | | GGATACCTATATCCACTGGGTGAGGCAGGCTCCAGGAA |
| | | AGGGCCTGGAGTGGGTGGCAAGGATTTACCCTACTAAT |
| | | GGATATACACGCTACGCTGATTCCGTGAAGGGACGCTTT |
| | | ACAATCTCAGCAGATACATCCAAAAACACGGCCTATTTA |
| | | CAGATGAATAGTTTGCGGGCCGAAGACACGGCTGTATA |
| | | CTATTGTTCTCGGTGGGGGGGCGATGGATTTTATGCGAT |
| | | GGATTACTGGGGCCAGGGCACCCTGGTAACCGTGTCAA |
| | | GCGGCTCAACATCCGGGTCCGGTAAGCCGGGCTCCGGC |
| | | GAGGGGTCTACAAAGGGAGATATACAGATGACACAGTC |
| | | CCCCAGTTCCCTGTCCGCCTCAGTGGGAGACCGAGTGAC |
| | | GATTACCTGTCGTGCCAGCCAGGACGTCAATACCGCCGT |
| | | CGCTTGGTATCAGCAAAAACCAGGCAAGGCCCCGAAAC |
| | | TATTGATCTACAGTGCCTCTTTTCTGTACTCCGGGGTGCC |
| | | GAGCAGATTTAGTGGCTCCAGGAGCGGAACCGATTTCA |
| | | CCCTAACCATTTCCAGTTTGCAGCCAGAGGATTTCGCGA |
| | | CCTATTACTGCCAGCAACACTACACCACACCGCCAACTT |
| | | TCGGACAAGGAACCAAGGTTGAAATCAAAATTGAAGTT |
| | | ATGTATCCTCCTCCTTACCTAGACAATGAGAAGAGCAAT |
| | | GGAACCATTATCCATGTGAAAGGGAAACACCTTTGTCCA |
| | | AGTCCCCTATTTCCCGGGCCTTCGAAGCCCTTTTGGGTG |
| | | CTGGTGGTGGTTGGTGGAGTCCTGGCTTGCTATAGCTTG |
| | | CTAGTAACAGTAGCGTTTATTATTTTCTGGGTGAAACGC |
| | | GGCCGCAAGAAACTGCTCTACATCTTTAAACAGCCGTTC |
| | | ATGAGGCCTGTGCAGACAACGCAGGAAGAGGATGGCTG |
| | | TAGTTGTCGGTTTCCGGAAGAGGAAGAGGGGGGCTGCG |
| | | AGTTGCGTGTCAAATTTTCTCGGTCTGCCGACGCCCCCG |
| | | CGTACCAGCAAGGGCAGAACCAGCTTTATAATGAGCTG |
| | | AATCTTGGACGACGGGAGGAATATGACGTGCTTGACAA |
| | | GAGGCGAGGTAGGGACCCTGAGATGGGGGGAAAACCTC |
| | | GGAGGAAAAACCCACAGGAAGGCCTGTATAACGAACTG |
| | | CAGAAGGACAAGATGGCTGAAGCCTACTCTGAGATTGG |
| | | AATGAAAGGGGAACGCAGACGCGGCAAGGGCCATGATG |
| | | GCCTCTACCAAGGTCTAAGCACTGCCACCAAGGACACCT |
| | | ATGACGCACTCCACATGCAAGCTCTACCTCCCCGTTGAT |
| | | AA |
| MC0294- | SEQ ID | MALPVTALLLPLALLLHAARPQVQLVQSGGGVVQPGGSLR |
| 3PF12_CD8 | NO: 30 | VSCAASGVTLSDYGMHWVRQAPGKGLEWMAFIRNDGSD |
| _LIR1_HE | | KYYADSVKGRFTISRDNSKKTVSLQMSSLRAEDTAVYYCA |
| R2 Protein | | KNGESGPLDYWYFDLWGRGTLVTVSSGGGGSGGGGSGGG |
| Sequence | | GSDIVMTQSPSFLSASVGDRVTITCRASHGINNYLAWYQQ |
| (VR294) | | KPGKAPKLLIYAASTLQSGVPSRFSGSGSGTEFTLTISSLQPE |
| | | DFATYYCQQYDSYPPTFGRGTKVEIKTTTPAPRPPTPAPTIA |
| | | SQPLSLRPEACRPAAGGAVHTRGLDFACDIYIWAPLAGTC |
| | | GVLLLSLVITLYCLRHRRQGKHWTSTQRKADFQHPAGAV |
| | | GPEPTDRGLQWRSSPAADAQEENLYAAVKHTQPEDGVEM |
| | | DTRSPHDEDPQAVTYAEVKHSRPRREMASPPSPLSGEFLDT |
| | | KDRQAEEDRQMDTEAAASEAPQDVTYAQLHSLTLRREAT |
| | | EPPPSQEGPSPAVPSIYATLAIHRRKRGSGEGRGSLLTCGDV |
| | | EENPGPMALPVTALLLPLALLLHAARPEVQLVESGGGLVQ |
| | | PGGSLRLSCAASGFNIKDTYIHWVRQAPGKGLEWVARIYP |
| | | TNGYTRYADSVKGRFTISADTSKNTAYLQMNSLRAEDTAV |
| | | YYCSRWGGDGFYAMDYWGQGTLVTVSSGSTSGSGKPGSG |
| | | EGSTKGDIQMTQSPSSLSASVGDRVTITCRASQDVNTAVA |
| | | WYQQKPGKAPKLLIYSASFLYSGVPSRFSGSRSGTDFTLTIS |
| | | SLQPEDFATYYCQQHYTTPPTFGQGTKVEIKIEVMYPPPYL |
| | | DNEKSNGTIIHVKGKHLCPSPLFPGPSKPFWVLVVVGGVLA |
| | | CYSLLVTVAFIIFWVKRGRKKLLYIFKQPFMRPVQTTQEED |
| | | GCSCRFPEEEEGGCELRVKFSRSADAPAYQQGQNQLYNEL |
| | | NLGRREEYDVLDKRRGRDPEMGGKPRRKNPQEGLYNELQ |
| | | KDKMAEAYSEIGMKGERRRGKGHDGLYQGLSTATKDTYD |
| | | ALHMQALPPR |
| MC0295- | SEQ ID | ATGGCACTGCCAGTGACCGCCCTGCTGCTGCCTCTGGCC |
| 3PF12_CD8 | NO: 31 | CTGCTGCTGCACGCAGCCAGACCCCAAGTGCAACTAGTC |
| _KIR2DL1_ | | CAATCAGGTGGAGGCGTCGTGCAACCTGGAGGGTCCCT |
| HER2 | | CCGCGTTAGCTGCGCCGCATCAGGCGTTACCTTGTCAGA |
| nucleotide | | CTACGGCATGCATTGGGTTAGGCAAGCCCCCGGCAAGG |
| Sequence | | GGCTCGAATGGATGGCTTTCATTCGGAATGACGGGAGC |
| (VR295) | | GATAAATATTACGCGGATTCAGTTAAAGGGCGGTTCACC |
| | | ATCAGCCGCGACAATAGCAAAAAGACGGTCTCCTTACA |
| | | GATGTCCAGCTTGCGGGCCGAAGACACGGCTGTATACTA |
| | | TTGTGCTAAAAATGGCGAGAGCGGCCCCCTGGATTACTG |
| | | GTACTTTGACCTGTGGGGCAGAGGCACCCTGGTCACGGT |
| | | GTCCTCTGGAGGAGGAGGCTCCGGAGGAGGAGGCTCTG |
| | | GCGGCGGCGGCAGCGACATTGTAATGACCCAGTCACCC |
| | | TCCTTCCTTAGTGCCTCAGTCGGAGACCGCGTGACTATC |
| | | ACTTGTCGTGCCTCACACGGAATTAATAACTACCTCGCT |
| | | TGGTATCAGCAAAAACCAGGCAAGGCCCCGAAACTATT |
| | | GATCTACGCCGCATCTACTCTGCAGAGCGGAGTACCGAG |
| | | CAGATTTAGTGGTTCCGGCAGCGGAACCGAGTTCACCCT |
| | | AACCATTTCCAGTTTGCAGCCAGAGGATTTCGCGACCTA |
| | | TTACTGCCAGCAATACGATTCATACCCGCCAACTTTCGG |
| | | AAGAGGTACCAAGGTTGAAATCAAGACTACGACCCCAG |
| | | CACCTAGACCTCCCACCCCAGCTCCAACTATAGCTTCCC |
| | | AGCCATTGTCTCTCCGGCCAGAGGCGTGTCGACCAGCCG |
| | | CTGGAGGGGCCGTTCATACAAGAGGACTCGATTTCGCTT |
| | | GCGATATCTACATATGGGCCCCTCTTGCCGGGACATGCG |
| | | GTGTCCTGCTTCTAAGCTTGGTTATTACCCTCTATTGCCA |
| | | TAGGTGGTGCTCAAACAAAAAGAATGCTGCCGTCATGG |
| | | ACCAGGAGAGCGCGGGCAATCGGACCGCAAACTCAGAG |
| | | GACTCAGATGAACAAGATCCACAGGAAGTGACCTACAC |
| | | TCAGCTGAACCATTGTGTGTTTACACAGCGCAAGATTAC |
| | | TCGTCCAAGCCAGCGTCCTAAGACCCCCCCGACCGATAT |
| | | CATTGTGTATACCGAGCTTCCTAATGCCGAATCCCGCAG |
| | | CAAGGTGGTCTCCTGCCCGCGGAGAAAGCGTGGATCCG |
| | | GGGAAGGCCGAGGCTCCCTTCTAACATGTGGAGATGTC |
| | | GAGGAAAACCCTGGCCCTATGGCGCTGCCAGTCACTGC |
| | | ATTGTTATTGCCTCTGGCCCTGCTTCTCCATGCGGCGCGC |
| | | CCAGAAGTGCAGCTGGTCGAGAGCGGAGGCGGACTGGT |
| | | TCAACCCGGAGGCAGCTTGAGACTGTCCTGCGCGGCCA |
| | | GCGGCTTCAACATCAAGGATACCTATATCCACTGGGTGA |
| | | GGCAGGCTCCAGGAAAGGGCCTGGAGTGGGTGGCAAGG |
| | | ATTTACCCTACTAATGGATATACACGCTACGCTGATTCC |
| | | GTGAAGGGACGCTTTACAATCTCAGCAGATACATCCAA |
| | | AAACACGGCCTATTTACAGATGAATAGTTTGCGGGCCGA |
| | | AGACACGGCTGTATACTATTGTTCTCGGTGGGGGGGCGA |
| | | TGGATTTTATGCGATGGATTACTGGGGCCAGGGCACCCT |
| | | GGTAACCGTGTCAAGCGGCTCAACATCCGGGTCCGGTA |
| | | AGCCGGGCTCCGGCGAGGGGTCTACAAAGGGAGATATA |
| | | CAGATGACACAGTCCCCCAGTTCCCTGTCCGCCTCAGTG |
| | | GGAGACCGAGTGACGATTACCTGTCGTGCCAGCCAGGA |
| | | CGTCAATACCGCCGTCGCTTGGTATCAGCAAAAACCAGG |
| | | CAAGGCCCCGAAACTATTGATCTACAGTGCCTCTTTTCT |
| | | GTACTCCGGGGTGCCGAGCAGATTTAGTGGCTCCAGGA |
| | | GCGGAACCGATTTCACCCTAACCATTTCCAGTTTGCAGC |
| | | CAGAGGATTTCGCGACCTATTACTGCCAGCAACACTACA |
| | | CCACACCGCCAACTTTCGGACAAGGAACCAAGGTTGAA |
| | | ATCAAAATTGAAGTTATGTATCCTCCTCCTTACCTAGAC |
| | | AATGAGAAGAGCAATGGAACCATTATCCATGTGAAAGG |
| | | GAAACACCTTTGTCCAAGTCCCCTATTTCCCGGGCCTTC |
| | | GAAGCCCTTTTGGGTGCTGGTGGTGGTTGGTGGAGTCCT |
| | | GGCTTGCTATAGCTTGCTAGTAACAGTAGCGTTTATTAT |
| | | TTTCTGGGTGAAACGCGGCCGCAAGAAACTGCTCTACAT |
| | | CTTTAAACAGCCGTTCATGAGGCCTGTGCAGACAACGCA |
| | | GGAAGAGGATGGCTGTAGTTGTCGGTTTCCGGAAGAGG |
| | | AAGAGGGGGGCTGCGAGTTGCGTGTCAAATTTTCTCGGT |
| | | CTGCCGACGCCCCCGCGTACCAGCAAGGGCAGAACCAG |
| | | CTTTATAATGAGCTGAATCTTGGACGACGGGAGGAATAT |
| | | GACGTGCTTGACAAGAGGCGAGGTAGGGACCCTGAGAT |
| | | GGGGGGAAAACCTCGGAGGAAAAACCCACAGGAAGGC |
| | | CTGTATAACGAACTGCAGAAGGACAAGATGGCTGAAGC |
| | | CTACTCTGAGATTGGAATGAAAGGGGAACGCAGACGCG |
| | | GCAAGGGCCATGATGGCCTCTACCAAGGTCTAAGCACT |
| | | GCCACCAAGGACACCTATGACGCACTCCACATGCAAGC |
| | | TCTACCTCCCCGTTGATAA |
| MC0295- | SEQ ID | MALPVTALLLPLALLLHAARPQVQLVQSGGGVVQPGGSLR |
| 3PF12_CD8 | NO: 32 | VSCAASGVTLSDYGMHWVRQAPGKGLEWMAFIRNDGSD |
| _KIR2DL1_ | | KYYADSVKGRFTISRDNSKKTVSLQMSSLRAEDTAVYYCA |
| HER2 | | KNGESGPLDYWYFDLWGRGTLVTVSSGGGGSGGGGSGGG |
| Protein | | GSDIVMTQSPSFLSASVGDRVTITCRASHGINNYLAWYQQ |
| Sequence | | KPGKAPKLLIYAASTLQSGVPSRFSGSGSGTEFTLTISSLQPE |
| (VR295) | | DFATYYCQQYDSYPPTFGRGTKVEIKTTTPAPRPPTPAPTIA |
| | | SQPLSLRPEACRPAAGGAVHTRGLDFACDIYIWAPLAGTC |
| | | GVLLLSLVITLYCHRWCSNKKNAAVMDQESAGNRTANSE |
| | | DSDEQDPQEVTYTQLNHCVFTQRKITRPSQRPKTPPTDIIVY |
| | | TELPNAESRSKVVSCPRRKRGSGEGRGSLLTCGDVEENPGP |
| | | MALPVTALLLPLALLLHAARPEVQLVESGGGLVQPGGSLR |
| | | LSCAASGFNIKDTYIHWVRQAPGKGLEWVARIYPTNGYTR |
| | | YADSVKGRFTISADTSKNTAYLQMNSLRAEDTAVYYCSR |
| | | WGGDGFYAMDYWGQGTLVTVSSGSTSGSGKPGSGEGSTK |
| | | GDIQMTQSPSSLSASVGDRVTITCRASQDVNTAVAWYQQK |
| | | PGKAPKLLIYSASFLYSGVPSRFSGSRSGTDFTLTISSLQPED |
| | | FATYYCQQHYTTPPTFGQGTKVEIKIEVMYPPPYLDNEKSN |
| | | GTIIHVKGKHLCPSPLFPGPSKPFWVLVVVGGVLACYSLLV |
| | | TVAFIIFWVKRGRKKLLYIFKQPFMRPVQTTQEEDGCSCRF |
| | | PEEEEGGCELRVKFSRSADAPAYQQGQNQLYNELNLGRRE |
| | | EYDVLDKRRGRDPEMGGKPRRKNPQEGLYNELQKDKMA |
| | | EAYSEIGMKGERRRGKGHDGLYQGLSTATKDTYDALHMQ |
| | | ALPPR |
| MC0296- | SEQ ID | ATGGCACTGCCAGTGACCGCCCTGCTGCTGCCTCTGGCC |
| BB7.2_CD8 | NO: 33 | CTGCTGCTGCACGCAGCCAGACCCCAGGTGCAGCTGCA |
| _LIR1_HE | | GCAGTCTGGACCTGAGCTGGTGAAGCCAGGAGCCTCCG |
| R2 | | TGAAGATGTCTTGCAAGGCCAGCGGCTACACCTTCACAT |
| nucleotide | | CTTATCACATCCAGTGGGTGAAGCAGCGGCCCGGACAG |
| Sequence | | GGCCTGGAGTGGATCGGATGGATCTACCCAGGCGACGG |
| (VR296) | | CTCCACACAGTATAACGAGAAGTTCAAGGGCAAGACCA |
| | | CACTGACCGCCGATAAGAGCAGCAGCACCGCCTACATG |
| | | CTGCTGAGCAGCCTGACCAGCGAGGACAGCGCCATCTA |
| | | CTTTTGCGCCAGGGAGGGCACATACTATGCTATGGACTA |
| | | TTGGGGCCAGGGCACCAGCGTGACAGTGTCTAGCGGAG |
| | | GAGGAGGCTCCGGAGGAGGAGGCTCTGGCGGCGGCGGC |
| | | AGCGACGTGCTGATGACCCAGACACCACTGAGCCTGCC |
| | | CGTGAGCCTGGGCGATCAGGTGAGCATCTCCTGTAGATC |
| | | CTCTCAGAGCATCGTGCACTCCAACGGCAATACCTACCT |
| | | GGAGTGGTATCTGCAGAAGCCAGGCCAGTCCCCCAAGC |
| | | TGCTGATCTATAAGGTGTCTAATCGGTTCAGCGGCGTGC |
| | | CTGACAGATTTTCTGGCAGCGGCTCCGGCACCGACTTCA |
| | | CCCTGAAGATCAGCCGGGTGGAGGCAGAGGATCTGGGC |
| | | GTGTACTATTGTTTCCAGGGCTCCCACGTGCCACGCACC |
| | | TTTGGCGGCGGTACCAAGCTGGAGATCAAGACTACGAC |
| | | CCCAGCACCTAGACCTCCCACCCCAGCTCCAACTATAGC |
| | | TTCCCAGCCATTGTCTCTCCGGCCAGAGGCGTGTCGACC |
| | | AGCCGCTGGAGGGGCCGTTCATACAAGAGGACTCGATT |
| | | TCGCTTGCGATATCTACATATGGGCCCCTCTTGCCGGGA |
| | | CATGCGGTGTCCTGCTTCTAAGCTTGGTTATTACCCTCTA |
| | | TTGCCTGCGCCACAGGAGACAGGGCAAGCACTGGACCA |
| | | GCACCCAGCGGAAGGCCGACTTTCAGCACCCTGCCGGC |
| | | GCCGTGGGCCCTGAGCCTACCGACAGGGGCCTGCAGTG |
| | | GAGGAGCTCCCCAGCCGCCGATGCCCAGGAGGAGAATC |
| | | TGTACGCCGCCGTGAAGCACACCCAGCCAGAGGACGGC |
| | | GTGGAGATGGACACCCGCTCCCCACACGACGAGGATCC |
| | | ACAGGCCGTGACCTACGCCGAGGTGAAGCACAGCCGCC |
| | | CCAGACGCGAGATGGCCAGCCCACCCAGCCCCCTGTCC |
| | | GGCGAGTTCCTGGACACCAAGGACAGGCAGGCCGAGGA |
| | | GGACCGGCAGATGGACACCGAGGCCGCCGCCTCCGAGG |
| | | CCCCCCAGGACGTGACCTACGCCCAGCTGCACTCCCTGA |
| | | CCCTGCGGAGAGAGGCCACCGAGCCCCCACCCAGCCAG |
| | | GAGGGCCCCTCCCCCGCCGTGCCTAGCATCTACGCCACC |
| | | CTGGCCATCCACCGGAGAAAGCGTGGATCCGGGGAAGG |
| | | CCGAGGCTCCCTTCTAACATGTGGAGATGTCGAGGAAA |
| | | ACCCTGGCCCTATGGCGCTGCCAGTCACTGCATTGTTAT |
| | | TGCCTCTGGCCCTGCTTCTCCATGCGGCGCGCCCAGAAG |
| | | TGCAGCTGGTCGAGAGCGGAGGCGGACTGGTTCAACCC |
| | | GGAGGCAGCTTGAGACTGTCCTGCGCGGCCAGCGGCTTC |
| | | AACATCAAGGATACCTATATCCACTGGGTGAGGCAGGC |
| | | TCCAGGAAAGGGCCTGGAGTGGGTGGCAAGGATTTACC |
| | | CTACTAATGGATATACACGCTACGCTGATTCCGTGAAGG |
| | | GACGCTTTACAATCTCAGCAGATACATCCAAAAACACG |
| | | GCCTATTTACAGATGAATAGTTTGCGGGCCGAAGACACG |
| | | GCTGTATACTATTGTTCTCGGTGGGGGGGCGATGGATTT |
| | | TATGCGATGGATTACTGGGGCCAGGGCACCCTGGTAACC |
| | | GTGTCAAGCGGCTCAACATCCGGGTCCGGTAAGCCGGG |
| | | CTCCGGCGAGGGGTCTACAAAGGGAGATATACAGATGA |
| | | CACAGTCCCCCAGTTCCCTGTCCGCCTCAGTGGGAGACC |
| | | GAGTGACGATTACCTGTCGTGCCAGCCAGGACGTCAATA |
| | | CCGCCGTCGCTTGGTATCAGCAAAAACCAGGCAAGGCC |
| | | CCGAAACTATTGATCTACAGTGCCTCTTTTCTGTACTCCG |
| | | GGGTGCCGAGCAGATTTAGTGGCTCCAGGAGCGGAACC |
| | | GATTTCACCCTAACCATTTCCAGTTTGCAGCCAGAGGAT |
| | | TTCGCGACCTATTACTGCCAGCAACACTACACCACACCG |
| | | CCAACTTTCGGACAAGGAACCAAGGTTGAAATCAAAAT |
| | | TGAAGTTATGTATCCTCCTCCTTACCTAGACAATGAGAA |
| | | GAGCAATGGAACCATTATCCATGTGAAAGGGAAACACC |
| | | TTTGTCCAAGTCCCCTATTTCCCGGGCCTTCGAAGCCCTT |
| | | TTGGGTGCTGGTGGTGGTTGGTGGAGTCCTGGCTTGCTA |
| | | TAGCTTGCTAGTAACAGTAGCGTTTATTATTTTCTGGGTG |
| | | AAACGCGGCCGCAAGAAACTGCTCTACATCTTTAAACA |
| | | GCCGTTCATGAGGCCTGTGCAGACAACGCAGGAAGAGG |
| | | ATGGCTGTAGTTGTCGGTTTCCGGAAGAGGAAGAGGGG |
| | | GGCTGCGAGTTGCGTGTCAAATTTTCTCGGTCTGCCGAC |
| | | GCCCCCGCGTACCAGCAAGGGCAGAACCAGCTTTATAA |
| | | TGAGCTGAATCTTGGACGACGGGAGGAATATGACGTGC |
| | | TTGACAAGAGGCGAGGTAGGGACCCTGAGATGGGGGGA |
| | | AAACCTCGGAGGAAAAACCCACAGGAAGGCCTGTATAA |
| | | CGAACTGCAGAAGGACAAGATGGCTGAAGCCTACTCTG |
| | | AGATTGGAATGAAAGGGGAACGCAGACGCGGCAAGGG |
| | | CCATGATGGCCTCTACCAAGGTCTAAGCACTGCCACCAA |
| | | GGACACCTATGACGCACTCCACATGCAAGCTCTACCTCC |
| | | CCGTTGATAA |
| MC0296- | SEQ ID | MALPVTALLLPLALLLHAARPQVQLQQSGPELVKPGASVK |
| BB7.2_CD8 | NO: 34 | MSCKASGYTFTSYHIQWVKQRPGQGLEWIGWIYPGDGSTQ |
| _LIR1_HE | | YNEKFKGKTTLTADKSSSTAYMLLSSLTSEDSAIYFCAREG |
| R2 protein | | TYYAMDYWGQGTSVTVSSGGGGSGGGGSGGGGSDVLMT |
| Sequence | | QTPLSLPVSLGDQVSISCRSSQSIVHSNGNTYLEWYLQKPG |
| (VR296) | | QSPKLLIYKVSNRFSGVPDRFSGSGSGTDFTLKISRVEAEDL |
| | | GVYYCFQGSHVPRTFGGGTKLEIKTTTPAPRPPTPAPTIASQ |
| | | PLSLRPEACRPAAGGAVHTRGLDFACDIYIWAPLAGTCGV |
| | | LLLSLVITLYCLRHRRQGKHWTSTQRKADFQHPAGAVGPE |
| | | PTDRGLQWRSSPAADAQEENLYAAVKHTQPEDGVEMDTR |
| | | SPHDEDPQAVTYAEVKHSRPRREMASPPSPLSGEFLDTKDR |
| | | QAEEDRQMDTEAAASEAPQDVTYAQLHSLTLRREATEPPP |
| | | SQEGPSPAVPSIYATLAIHRRKRGSGEGRGSLLTCGDVEEN |
| | | PGPMALPVTALLLPLALLLHAARPEVQLVESGGGLVQPGG |
| | | SLRLSCAASGFNIKDTYIHWVRQAPGKGLEWVARIYPTNG |
| | | YTRYADSVKGRFTISADTSKNTAYLQMNSLRAEDTAVYYC |
| | | SRWGGDGFYAMDYWGQGTLVTVSSGSTSGSGKPGSGEGS |
| | | TKGDIQMTQSPSSLSASVGDRVTITCRASQDVNTAVAWYQ |
| | | QKPGKAPKLLIYSASFLYSGVPSRFSGSRSGTDFTLTISSLQP |
| | | EDFATYYCQQHYTTPPTFGQGTKVEIKIEVMYPPPYLDNEK |
| | | SNGTIIHVKGKHLCPSPLFPGPSKPFWVLVVVGGVLACYSL |
| | | LVTVAFIIFWVKRGRKKLLYIFKQPFMRPVQTTQEEDGCSC |
| | | RFPEEEEGGCELRVKFSRSADAPAYQQGQNQLYNELNLGR |
| | | REEYDVLDKRRGRDPEMGGKPRRKNPQEGLYNELQKDK |
| | | MAEAYSEIGMKGERRRGKGHDGLYQGLSTATKDTYDALH |
| | | MQALPPR |
| MC0297- | SEQ ID | ATGGCACTGCCAGTGACCGCCCTGCTGCTGCCTCTGGCC |
| BB7.2_CD8 | NO: 35 | CTGCTGCTGCACGCAGCCAGACCCCAGGTGCAGCTGCA |
| _KIR2DL1_ | | GCAGTCTGGACCTGAGCTGGTGAAGCCAGGAGCCTCCG |
| HER2- | | TGAAGATGTCTTGCAAGGCCAGCGGCTACACCTTCACAT |
| nucleic acid | | CTTATCACATCCAGTGGGTGAAGCAGCGGCCCGGACAG |
| (VR297) | | GGCCTGGAGTGGATCGGATGGATCTACCCAGGCGACGG |
| | | CTCCACACAGTATAACGAGAAGTTCAAGGGCAAGACCA |
| | | CACTGACCGCCGATAAGAGCAGCAGCACCGCCTACATG |
| | | CTGCTGAGCAGCCTGACCAGCGAGGACAGCGCCATCTA |
| | | CTTTTGCGCCAGGGAGGGCACATACTATGCTATGGACTA |
| | | TTGGGGCCAGGGCACCAGCGTGACAGTGTCTAGCGGAG |
| | | GAGGAGGCTCCGGAGGAGGAGGCTCTGGCGGCGGCGGC |
| | | AGCGACGTGCTGATGACCCAGACACCACTGAGCCTGCC |
| | | CGTGAGCCTGGGCGATCAGGTGAGCATCTCCTGTAGATC |
| | | CTCTCAGAGCATCGTGCACTCCAACGGCAATACCTACCT |
| | | GGAGTGGTATCTGCAGAAGCCAGGCCAGTCCCCCAAGC |
| | | TGCTGATCTATAAGGTGTCTAATCGGTTCAGCGGCGTGC |
| | | CTGACAGATTTTCTGGCAGCGGCTCCGGCACCGACTTCA |
| | | CCCTGAAGATCAGCCGGGTGGAGGCAGAGGATCTGGGC |
| | | GTGTACTATTGTTTCCAGGGCTCCCACGTGCCACGCACC |
| | | TTTGGCGGCGGTACCAAGCTGGAGATCAAGACTACGAC |
| | | CCCAGCACCTAGACCTCCCACCCCAGCTCCAACTATAGC |
| | | TTCCCAGCCATTGTCTCTCCGGCCAGAGGCGTGTCGACC |
| | | AGCCGCTGGAGGGGCCGTTCATACAAGAGGACTCGATT |
| | | TCGCTTGCGATATCTACATATGGGCCCCTCTTGCCGGGA |
| | | CATGCGGTGTCCTGCTTCTAAGCTTGGTTATTACCCTCTA |
| | | TTGCCATAGGTGGTGCTCAAACAAAAAGAATGCTGCCGT |
| | | CATGGACCAGGAGAGCGCGGGCAATCGGACCGCAAACT |
| | | CAGAGGACTCAGATGAACAAGATCCACAGGAAGTGACC |
| | | TACACTCAGCTGAACCATTGTGTGTTTACACAGCGCAAG |
| | | ATTACTCGTCCAAGCCAGCGTCCTAAGACCCCCCCGACC |
| | | GATATCATTGTGTATACCGAGCTTCCTAATGCCGAATCC |
| | | CGCAGCAAGGTGGTCTCCTGCCCGCGGAGAAAGCGTGG |
| | | ATCCGGGGAAGGCCGAGGCTCCCTTCTAACATGTGGAG |
| | | ATGTCGAGGAAAACCCTGGCCCTATGGCGCTGCCAGTCA |
| | | CTGCATTGTTATTGCCTCTGGCCCTGCTTCTCCATGCGGC |
| | | GCGCCCAGAAGTGCAGCTGGTCGAGAGCGGAGGCGGAC |
| | | TGGTTCAACCCGGAGGCAGCTTGAGACTGTCCTGCGCGG |
| | | CCAGCGGCTTCAACATCAAGGATACCTATATCCACTGGG |
| | | TGAGGCAGGCTCCAGGAAAGGGCCTGGAGTGGGTGGCA |
| | | AGGATTTACCCTACTAATGGATATACACGCTACGCTGAT |
| | | TCCGTGAAGGGACGCTTTACAATCTCAGCAGATACATCC |
| | | AAAAACACGGCCTATTTACAGATGAATAGTTTGCGGGCC |
| | | GAAGACACGGCTGTATACTATTGTTCTCGGTGGGGGGGC |
| | | GATGGATTTTATGCGATGGATTACTGGGGCCAGGGCACC |
| | | CTGGTAACCGTGTCAAGCGGCTCAACATCCGGGTCCGGT |
| | | AAGCCGGGCTCCGGCGAGGGGTCTACAAAGGGAGATAT |
| | | ACAGATGACACAGTCCCCCAGTTCCCTGTCCGCCTCAGT |
| | | GGGAGACCGAGTGACGATTACCTGTCGTGCCAGCCAGG |
| | | ACGTCAATACCGCCGTCGCTTGGTATCAGCAAAAACCAG |
| | | GCAAGGCCCCGAAACTATTGATCTACAGTGCCTCTTTTC |
| | | TGTACTCCGGGGTGCCGAGCAGATTTAGTGGCTCCAGGA |
| | | GCGGAACCGATTTCACCCTAACCATTTCCAGTTTGCAGC |
| | | CAGAGGATTTCGCGACCTATTACTGCCAGCAACACTACA |
| | | CCACACCGCCAACTTTCGGACAAGGAACCAAGGTTGAA |
| | | ATCAAAATTGAAGTTATGTATCCTCCTCCTTACCTAGAC |
| | | AATGAGAAGAGCAATGGAACCATTATCCATGTGAAAGG |
| | | GAAACACCTTTGTCCAAGTCCCCTATTTCCCGGGCCTTC |
| | | GAAGCCCTTTTGGGTGCTGGTGGTGGTTGGTGGAGTCCT |
| | | GGCTTGCTATAGCTTGCTAGTAACAGTAGCGTTTATTAT |
| | | TTTCTGGGTGAAACGCGGCCGCAAGAAACTGCTCTACAT |
| | | CTTTAAACAGCCGTTCATGAGGCCTGTGCAGACAACGCA |
| | | GGAAGAGGATGGCTGTAGTTGTCGGTTTCCGGAAGAGG |
| | | AAGAGGGGGGCTGCGAGTTGCGTGTCAAATTTTCTCGGT |
| | | CTGCCGACGCCCCCGCGTACCAGCAAGGGCAGAACCAG |
| | | CTTTATAATGAGCTGAATCTTGGACGACGGGAGGAATAT |
| | | GACGTGCTTGACAAGAGGCGAGGTAGGGACCCTGAGAT |
| | | GGGGGGAAAACCTCGGAGGAAAAACCCACAGGAAGGC |
| | | CTGTATAACGAACTGCAGAAGGACAAGATGGCTGAAGC |
| | | CTACTCTGAGATTGGAATGAAAGGGGAACGCAGACGCG |
| | | GCAAGGGCCATGATGGCCTCTACCAAGGTCTAAGCACT |
| | | GCCACCAAGGACACCTATGACGCACTCCACATGCAAGC |
| | | TCTACCTCCCCGTTGATAA |
| MC00297: | SEQ ID | MALPVTALLLPLALLLHAARPQVQLQQSGPELVKPGASVK |
| BB7.2_CD8 | NO: 36 | MSCKASGYTFTSYHIQWVKQRPGQGLEWIGWIYPGDGSTQ |
| _KIR2DL1_ | | YNEKFKGKTTLTADKSSSTAYMLLSSLTSEDSAIYFCAREG |
| HER2 | | TYYAMDYWGQGTSVTVSSGGGGSGGGGSGGGGSDVLMT |
| protein | | QTPLSLPVSLGDQVSISCRSSQSIVHSNGNTYLEWYLQKPG |
| Sequence | | QSPKLLIYKVSNRFSGVPDRFSGSGSGTDFTLKISRVEAEDL |
| (VR297) | | GVYYCFQGSHVPRTFGGGTKLEIKTTTPAPRPPTPAPTIASQ |
| | | PLSLRPEACRPAAGGAVHTRGLDFACDIYIWAPLAGTCGV |
| | | LLLSLVITLYCHRWCSNKKNAAVMDQESAGNRTANSEDS |
| | | DEQDPQEVTYTQLNHCVFTQRKITRPSQRPKTPPTDIIVYTE |
| | | LPNAESRSKVVSCPRRKRGSGEGRGSLLTCGDVEENPGPM |
| | | ALPVTALLLPLALLLHAARPEVQLVESGGGLLVQPGGSLRL |
| | | SCAASGFNIKDTYIHWVRQAPGKGLEWVARIYPTNGYTRY |
| | | ADSVKGRFTISADTSKNTAYLQMNSLRAEDTAVYYCSRW |
| | | GGDGFYAMDYWGQGTLVTVSSGSTSGSGKPGSGEGSTKG |
| | | DIQMTQSPSSLSASVGDRVTITCRASQDVNTAVAWYQQKP |
| | | GKAPKLLIYSASFLYSGVPSRFSGSRSGTDFTLTISSLQPEDF |
| | | ATYYCQQHYTTPPTFGQGTKVEIKIEVMYPPPYLDNEKSN |
| | | GTIIHVKGKHLCPSPLFPGPSKPFWVLVVVGGVLACYSLLV |
| | | TVAFIIFWVKRGRKKLLYIFKQPFMRPVQTTQEEDGCSCRF |
| | | PEEEEGGCELRVKFSRSADAPAYQQGQNQLYNELNLGRRE |
| | | EYDVLDKRRGRDPEMGGKPRRKNPQEGLYNELQKDKMA |
| | | EAYSEIGMKGERRRGKGHDGLYQGLSTATKDTYDALHMQ |
| | | ALPPR |
| MC0421 | SEQ ID | ATGGCGCTGCCAGTCACTGCATTGTTATTGCCTCTGGCC |
| HzBB7.2.2_ | NO: 275 | CTGCTTCTCCATGCGGCAAGGCCACAGGTGCAACTGGTT |
| LIR1(52)_2 | | CAATCTGGTGCTGAGGTGAAAAAGCCCGGCGCATCCGT |
| A_HER2 | | GAAAGTGAGCTGTAAGGCATCAGGGTACACCTTCACCA |
| nucleotide | | GCTATCACATACAATGGGTCCGCCAGGCCCCCGGACAG |
| sequence | | AGGTTGGAATGGATTGGGTGGATTTACCCGGGTGACGG |
| (VR421) | | CTCAACCCAGTACAATGAGAAGTTCAAGGGCAGGGTGA |
| | | CTATCACACGCGATACCTCCGCGAGCACAGCTTACATGG |
| | | AGTTATCTAGCCTGAGATCCGAAGATACGGCGGTGTATT |
| | | ACTGCGCGCGGGAAGGGACCTACTATGCCATGGACTATT |
| | | GGGGACAAGGGACCCTGGTTACCGTGAGTTCTGGGGGC |
| | | GGGGGTTCCGGGGGAGGGGGATCTGGGGGTGGAGGGAG |
| | | CGATGTGGTAATGACCCAGACACCTTTGTCTTTGAGTGT |
| | | CACCCCCGGACAGCCGGCAAGTATATCCTGTAGATCATC |
| | | CCAATCAATCGTGCACTCCAACGGAAACACATACTTGGA |
| | | ATGGTATCTCCAGAAACCTGGACAGTCCCCACAGTTGCT |
| | | CATCTACAAAGTGTCAAATCGCTTTTCAGGCGTGCCCGA |
| | | TCGTTTCAGCGGCTCAGGCTCCGGGACAGACTTTACATT |
| | | GAAGATTAGCCGCGTAGAGGCAGAGGATGTGGGCGTTT |
| | | ACTATTGTTTTCAAGGGTCACACGTGCCACGCACATTCG |
| | | GCGGCGGTACCAAGGTGGAAATTAAGCACCCCAGCGAC |
| | | CCGCTGGAGCTCGTTGTGTCCGGACCATCAGGGGGCCCG |
| | | AGTAGCCCTACAACCGGCCCCACTTCTACCAGTGGACCG |
| | | GAAGATCAACCACTTACACCAACGGGCAGCGACCCCCA |
| | | GTCAGGCCTAGGGCGCCACCTGGGTGTGGTCATCGGGAT |
| | | ACTGGTCGCTGTCATCCTGCTTCTGCTCCTTCTCTTGCTC |
| | | CTATTCCTAATCCTGCGCCACAGGAGACAGGGCAAGCA |
| | | CTGGACCAGCACCCAGCGGAAGGCCGACTTTCAGCACC |
| | | CTGCCGGCGCCGTGGGCCCTGAGCCTACCGACAGGGGC |
| | | CTGCAGTGGAGGAGCTCCCCAGCCGCCGATGCCCAGGA |
| | | GGAGAATCTGTACGCCGCCGTGAAGCACACCCAGCCAG |
| | | AGGACGGCGTGGAGATGGACACCCGCTCCCCACACGAC |
| | | GAGGACCCACAGGCCGTGACCTACGCCGAGGTGAAGCA |
| | | CAGCCGCCCCAGACGCGAGATGGCCAGCCCACCCAGCC |
| | | CCCTGTCCGGCGAGTTCCTGGACACCAAGGACAGGCAG |
| | | GCCGAGGAGGACCGGCAGATGGACACCGAGGCCGCCGC |
| | | CTCCGAGGCCCCCCAGGACGTGACCTACGCCCAGCTGCA |
| | | CTCCCTGACCCTGCGGAGAGAGGCCACCGAGCCCCCAC |
| | | CCAGCCAGGAGGGCCCCTCCCCCGCCGTGCCTAGCATCT |
| | | ACGCCACCCTGGCCATCCACCGGAGAAAGCGTGGATCC |
| | | GGGGAAGGCCGAGGCTCCCTTCTAACATGTGGAGATGT |
| | | CGAGGAAAACCCTGGCCCTATGGCGCTGCCAGTCACTGC |
| | | ATTGTTATTGCCTCTGGCCCTGCTTCTCCATGCGGCGCGC |
| | | CCAGACATCCAGATGACCCAATCCCCAAGCAGTCTCTCA |
| | | GCCAGCGTGGGAGACAGGGTTACAATCACGTGCCGCGC |
| | | CAGCCAGGACGTCAACACCGCTGTGGCTTGGTATCAGCA |
| | | AAAGCCCGGGAAGGCACCAAAGCTGCTTATTTATAGCG |
| | | CCTCCTTCTTGTATTCTGGAGTGCCATCCAGGTTTTCCGG |
| | | GTCACGTAGCGGGACTGACTTTACCCTCACCATATCCAG |
| | | CCTCCAGCCCGAGGATTTCGCCACCTATTACTGTCAGCA |
| | | ACACTACACGACTCCACCGACTTTTGGACAGGGCACTAA |
| | | AGTGGAGATTAAGGGCAGCACGAGTGGGAGTGGAAAGC |
| | | CCGGCAGCGGGGAGGGGTCTACCAAGGGAGAGGTCCAG |
| | | CTGGTTGAATCCGGAGGCGGGCTTGTGCAACCTGGAGG |
| | | CTCCCTGAGGCTTAGTTGTGCCGCGTCAGGATTCAACAT |
| | | TAAGGATACCTATATTCATTGGGTCCGACAAGCCCCGGG |
| | | CAAGGGCTTGGAGTGGGTGGCCAGAATCTATCCGACCA |
| | | ACGGATATACAAGGTACGCCGATTCTGTGAAAGGACGC |
| | | TTCACCATCAGCGCGGACACATCCAAAAACACAGCCTAT |
| | | CTGCAGATGAACTCCCTTCGCGCCGAGGATACAGCCGTG |
| | | TACTATTGTAGTCGGTGGGGAGGCGACGGCTTCTACGCG |
| | | ATGGACTATTGGGGACAAGGAACACTGGTGACTGTCAG |
| | | TAGCACTACGACCCCAGCACCTAGACCTCCCACCCCAGC |
| | | TCCAACTATAGCTTCCCAGCCATTGTCTCTCCGGCCAGA |
| | | GGCGTGTCGACCAGCCGCTGGAGGGGCCGTTCATACAA |
| | | GAGGACTCGATTTCGCTTGCGATATCTACATATGGGCCC |
| | | CTCTTGCCGGGACATGCGGTGTCCTGCTTCTAAGCTTGG |
| | | TTATTACCCTCTATTGCAAAAGAGGACGAAAGAAACTGC |
| | | TTTATATATTCAAGCAACCTTTCATGCGCCCCGTACAGA |
| | | CCACGCAGGAGGAAGATGGGTGTAGCTGTCGCTTCCCTG |
| | | AGGAAGAGGAAGGTGGATGCGAGTTGCGGGTGAAGTTC |
| | | AGTCGATCCGCCGATGCGCCTGCCTATCAGCAAGGGCA |
| | | GAACCAGCTTTATAACGAGTTAAACCTTGGCCGCCGGGA |
| | | AGAGTATGACGTGTTGGACAAGCGTCGCGGGAGAGACC |
| | | CTGAGATGGGCGGAAAACCAAGGAGAAAAAATCCACAG |
| | | GAAGGCTTATATAACGAGTTGCAGAAAGACAAGATGGC |
| | | CGAGGCATACTCCGAAATCGGAATGAAGGGCGAGCGAC |
| | | GGCGCGGCAAAGGCCACGATGGACTCTATCAGGGCTTA |
| | | AGCACCGCCACCAAAGACACCTACGATGCACTTCATATG |
| | | CAGGCACTCCCACCTAGATGATAA |
| MC0421 | SEQ ID | MALPVTALLLPLALLLHAARPQVQLVQSGAEVKKPGASV |
| HzBB7.2.2_ | NO: 276 | KVSCKASGYTFTSYHIQWVRQAPGQRLEWIGWIYPGDGST |
| LIR1(52)_2 | | QYNEKFKGRVTITRDTSASTAYMELSSLRSEDTAVYYCAR |
| A_HER2 | | EGTYYAMDYWGQGTLVTVSSGGGGSGGGGSGGGGSDVV |
| Protein | | MTQTPLSLSVTPGQPASISCRSSQSIVHSNGNTYLEWYLQK |
| Sequence | | PGQSPQLLIYKVSNRFSGVPDRFSGSGSGTDFTLKISRVEAE |
| (VR421) | | DVGVYYCFQGSHVPRTFGGGTKVEIKHPSDPLELVVSGPS |
| | | GGPSSPTTGPTSTSGPEDQPLTPTGSDPQSGLGRHLGVVIGI |
| | | LVAVILLLLLLLLLFLILRHRRQGKHWTSTQRKADFQHPAG |
| | | AVGPEPTDRGLQWRSSPAADAQEENLYAAVKHTQPEDGV |
| | | EMDTRSPHDEDPQAVTYAEVKHSRPRREMASPPSPLSGEFL |
| | | DTKDRQAEEDRQMDTEAAASEAPQDVTYAQLHSLTLRRE |
| | | ATEPPPSQEGPSPAVPSIYATLAIHRRKRGSGEGRGSLLTCG |
| | | DVEENPGPMALPVTALLLPLALLLHAARPDIQMTQSPSSLS |
| | | ASVGDRVTITCRASQDVNTAVAWYQQKPGKAPKLLIYSAS |
| | | FLYSGVPSRFSGSRSGTDFTLTISSLQPEDFATYYCQQHYTT |
| | | PPTFGQGTKVEIKGSTSGSGKPGSGEGSTKGEVQLVESGGG |
| | | LVQPGGSLRLSCAASGFNIKDTYIHWVRQAPGKGLEWVAR |
| | | IYPTNGYTRYADSVKGRFTISADTSKNTAYLQMNSLRAED |
| | | TAVYYCSRWGGDGFYAMDYWGQGTLVTVSSTTTPAPRPP |
| | | TPAPTIASQPLSLRPEACRPAAGGAVHTRGLDFACDIYIWA |
| | | PLAGTCGVLLLSLVITLYCKRGRKKLLYIFKQPFMRPVQTT |
| | | QEEDGCSCRFPEEEEGGCELRVKFSRSADAPAYQQGQNQL |
| | | YNELNLGRREEYDVLDKRRGRDPEMGGKPRRKNPQEGLY |
| | | NELQKDKMAEAYSEIGMKGERRRGKGHDGLYQGLSTATK |
| | | DTYDALHMQALPPR |
| MC00428 | SEQ ID | ATGGCGCTGCCAGTCACTGCATTGTTATTGCCTCTGGCC |
| HzBB7.2.1_ | NO: 277 | CTGCTTCTCCATGCGGCAAGGCCACAGGTGCAACTGGTT |
| LIR1(52)_ | | CAATCTGGTGCTGAGGTGAAAAAGCCCGGCAGCTCTGT |
| (IRESL)_H | | GAAAGTGAGCTGTAAGGCATCAGGGTATACCTTCACCA |
| ER2 | | GCTATCACATACAATGGGTCCGCCAGGCCCCCGGACAG |
| nucleotide | | GGATTGGAATGGATGGGGTGGATTTACCCGGGTGACGG |
| sequence | | CTCAACCCAGTACAATGAGAAGTTCAAGGGCAGGACAA |
| (VR428) | | CTATCACAGCCGATAAGTCCACGAGCACAGCTTACATGG |
| | | AGTTATCTAGCCTGAGATCCGAAGATACGGCGGTGTATT |
| | | ACTGCGCGCGGGAAGGGACCTACTATGCCATGGACTATT |
| | | GGGGACAAGGGACCCTGGTTACCGTGAGTTCTGGGGGC |
| | | GGGGGTTCCGGGGGAGGGGGATCTGGGGGTGGAGGGAG |
| | | CGATGTGGTAATGACCCAGACACCTTTGTCTTTGAGTGT |
| | | CACCCCCGGACAGCCGGCAAGTATATCCTGTAGATCATC |
| | | CCAATCAATCGTGCACTCCAACGGAAACACATACTTGGA |
| | | ATGGTATCTCCAGAAACCTGGACAGTCCCCACAGTTGCT |
| | | CATCTACAAAGTGTCAAATCGCTTTTCAGGCGTGCCCGA |
| | | TCGTTTCAGCGGCTCAGGCTCCGGGACAGACTTTACATT |
| | | GAAGATTAGCCGCGTAGAGGCAGAGGATGTGGGCGTTT |
| | | ACTATTGTTTTCAAGGGTCACACGTGCCACGCACATTCG |
| | | GCGGCGGTACCAAGGTGGAAATTAAGCACCCCAGCGAC |
| | | CCGCTGGAGCTCGTTGTGTCCGGACCATCAGGGGGCCCG |
| | | AGTAGCCCTACAACCGGCCCCACTTCTACCAGTGGACCG |
| | | GAAGATCAACCACTTACACCAACGGGCAGCGACCCCCA |
| | | GTCAGGCCTAGGGCGCCACCTGGGTGTGGTCATCGGGAT |
| | | ACTGGTCGCTGTCATCCTGCTTCTGCTCCTTCTCTTGCTC |
| | | CTATTCCTAATCCTGCGCCACAGGAGACAGGGCAAGCA |
| | | CTGGACCAGCACCCAGCGGAAGGCCGACTTTCAGCACC |
| | | CTGCCGGCGCCGTGGGCCCTGAGCCTACCGACAGGGGC |
| | | CTGCAGTGGAGGAGCTCCCCAGCCGCCGATGCCCAGGA |
| | | GGAGAATCTGTACGCCGCCGTGAAGCACACCCAGCCAG |
| | | AGGACGGCGTGGAGATGGACACCCGCTCCCCACACGAC |
| | | GAGGACCCACAGGCCGTGACCTACGCCGAGGTGAAGCA |
| | | CAGCCGCCCCAGACGCGAGATGGCCAGCCCACCCAGCC |
| | | CCCTGTCCGGCGAGTTCCTGGACACCAAGGACAGGCAG |
| | | GCCGAGGAGGACCGGCAGATGGACACCGAGGCCGCCGC |
| | | CTCCGAGGCCCCCCAGGACGTGACCTACGCCCAGCTGCA |
| | | CTCCCTGACCCTGCGGAGAGAGGCCACCGAGCCCCCAC |
| | | CCAGCCAGGAGGGCCCCTCCCCCGCCGTGCCTAGCATCT |
| | | ACGCCACCCTGGCCATCCACTGATAACCCCCCCCCCTAA |
| | | CGTTACTGGCCGAAGCCGCTTGGAATAAGGCCGGTGTGC |
| | | GTTTGTCTATATGTTATTTTCCACCATATTGCCGTCTTTT |
| | | GGCAATGTGAGGGCCCGGAAACCTGGCCCTGTCTTCTTG |
| | | ACGAGCATTCCTAGGGGTCTTTCCCCTCTCGCCAAAGGA |
| | | ATGCAAGGTCTGTTGAATGTCGTGAAGGAAGCAGTTCCT |
| | | CTGGAAGCTTCTTGAAGACAAACAACGTCTGTAGCGACC |
| | | CTTTGCAGGCAGCGGAACCCCCCACCTGGCGACAGGTG |
| | | CCTCTGCGGCCAAAAGCCACGTGTATAAGATACACCTGC |
| | | AAAGGCGGCACAACCCCAGTGCCACGTTGTGAGTTGGA |
| | | TAGTTGTGGAAAGAGTCAAATGGCTCTCCTCAAGCGTAT |
| | | TCAACAAGGGGCTGAAGGATGCCCAGAAGGTACCCCAT |
| | | TGTATGGGATCTGATCTGGGGCCTCGGTGCACATGCTTT |
| | | ACATGTGTTTAGTCGAGGTTAAAAAAACGTCTAGGCCCC |
| | | CCGAACCACGGGGACGTGGTTTTCCTTTGAAAAACACGA |
| | | TGATAATATGATGGCGCTGCCAGTCACTGCATTGTTATT |
| | | GCCTCTGGCCCTGCTTCTCCATGCGGCGCGCCCAGACAT |
| | | CCAGATGACCCAATCCCCAAGCAGTCTCTCAGCCAGCGT |
| | | GGGAGACAGGGTTACAATCACGTGCCGCGCCAGCCAGG |
| | | ACGTCAACACCGCTGTGGCTTGGTATCAGCAAAAGCCCG |
| | | GGAAGGCACCAAAGCTGCTTATTTATAGCGCCTCCTTCT |
| | | TGTATTCTGGAGTGCCATCCAGGTTTTCCGGGTCACGTA |
| | | GCGGGACTGACTTTACCCTCACCATATCCAGCCTCCAGC |
| | | CCGAGGATTTCGCCACCTATTACTGTCAGCAACACTACA |
| | | CGACTCCACCGACTTTTGGACAGGGCACTAAAGTGGAG |
| | | ATTAAGGGCAGCACGAGTGGGAGTGGAAAGCCCGGCAG |
| | | CGGGGAGGGGTCTACCAAGGGAGAGGTCCAGCTGGTTG |
| | | AATCCGGAGGCGGGCTTGTGCAACCTGGAGGCTCCCTG |
| | | AGGCTTAGTTGTGCCGCGTCAGGATTCAACATTAAGGAT |
| | | ACCTATATTCATTGGGTCCGACAAGCCCCGGGCAAGGGC |
| | | TTGGAGTGGGTGGCCAGAATCTATCCGACCAACGGATAT |
| | | ACAAGGTACGCCGATTCTGTGAAAGGACGCTTCACCATC |
| | | AGCGCGGACACATCCAAAAACACAGCCTATCTGCAGAT |
| | | GAACTCCCTTCGCGCCGAGGATACAGCCGTGTACTATTG |
| | | TAGTCGGTGGGGAGGCGACGGCTTCTACGCGATGGACT |
| | | ATTGGGGACAAGGAACACTGGTGACTGTCAGTAGCACT |
| | | ACGACCCCAGCACCTAGACCTCCCACCCCAGCTCCAACT |
| | | ATAGCTTCCCAGCCATTGTCTCTCCGGCCAGAGGCGTGT |
| | | CGACCAGCCGCTGGAGGGGCCGTTCATACAAGAGGACT |
| | | CGATTTCGCTTGCGATATCTACATATGGGCCCCTCTTGCC |
| | | GGGACATGCGGTGTCCTGCTTCTAAGCTTGGTTATTACC |
| | | CTCTATTGCAAAAGAGGACGAAAGAAACTGCTTTATATA |
| | | TTCAAGCAACCTTTCATGCGCCCCGTACAGACCACGCAG |
| | | GAGGAAGATGGGTGTAGCTGTCGCTTCCCTGAGGAAGA |
| | | GGAAGGTGGATGCGAGTTGCGGGTGAAGTTCAGTCGAT |
| | | CCGCCGATGCGCCTGCCTATCAGCAAGGGCAGAACCAG |
| | | CTTTATAACGAGTTAAACCTTGGCCGCCGGGAAGAGTAT |
| | | GACGTGTTGGACAAGCGTCGCGGGAGAGACCCTGAGAT |
| | | GGGCGGAAAACCAAGGAGAAAAAATCCACAGGAAGGC |
| | | TTATATAACGAGTTGCAGAAAGACAAGATGGCCGAGGC |
| | | ATACTCCGAAATCGGAATGAAGGGCGAGCGACGGCGCG |
| | | GCAAAGGCCACGATGGACTCTATCAGGGCTTAAGCACC |
| | | GCCACCAAAGACACCTACGATGCACTTCATATGCAGGC |
| | | ACTCCCACCTAGATGATAA |
| MC0428 | SEQ ID | MALPVTALLLPLALLLHAARPQVQLVQSGAEVKKPGSSVK |
| HzBB7.2.1_ | NO: 278 | VSCKASGYTFTSYHIQWVRQAPGQGLEWMGWIYPGDGST |
| LIR1(52)_ | | QYNEKFKGRTTITADKSTSTAYMELSSLRSEDTAVYYCAR |
| (IRESL)_H | | EGTYYAMDYWGQGTLVTVSSGGGGSGGGGSGGGGSDVV |
| ER2 Protein | | MTQTPLSLSVTPGQPASISCRSSQSIVHSNGNTYLEWYLQK |
| sequence | | PGQSPQLLIYKVSNRFSGVPDRFSGSGSGTDFTLKISRVEAE |
| (VR428) | | DVGVYYCFQGSHVPRTFGGGTKVEIKHPSDPLELVVSGPS |
| | | GGPSSPTTGPTSTSGPEDQPLTPTGSDPQSGLGRHLGVVIGI |
| | | LVAVILLLLLLLLLFLILRHRRQGKHWTSTQRKADFQHPAG |
| | | AVGPEPTDRGLQWRSSPAADAQEENLYAAVKHTQPEDGV |
| | | EMDTRSPHDEDPQAVTYAEVKHSRPRREMASPPSPLSGEFL |
| | | DTKDRQAEEDRQMDTEAAASEAPQDVTYAQLHSLTLRRE |
| | | ATEPPPSQEGPSPAVPSIYATLAIH* |
| | | MALPVTALLLPLALLLHAARPDIQMTQSPSSLSASVGDRVT |
| | | ITCRASQDVNTAVAWYQQKPGKAPKLLIYSASFLYSGVPS |
| | | RFSGSRSGTDFTLTISSLQPEDFATYYCQQHYTTPPTFGQGT |
| | | KVEIKGSTSGSGKPGSGEGSTKGEVQLVESGGGLVQPGGSL |
| | | RLSCAASGFNIKDTYIHWVRQAPGKGLEWVARIYPTNGYT |
| | | RYADSVKGRFTISADTSKNTAYLQMNSLRAEDTAVYYCSR |
| | | WGGDGFYAMDYWGQGTLVTVSSTTTPAPRPPTPAPTIASQ |
| | | PLSLRPEACRPAAGGAVHTRGLDFACDIYIWAPLAGTCGV |
| | | LLLSLVITLYCKRGRKKLLYIFKQPFMRPVQTTQEEDGCSC |
| | | RFPEEEEGGCELRVKFSRSADAPAYQQGQNQLYNELNLGR |
| | | REEYDVLDKRRGRDPEMGGKPRRKNPQEGLYNELQKDK |
| | | MAEAYSEIGMKGERRRGKGHDGLYQGLSTATKDTYDALH |
| | | MQALPPR* |
| MC0447 | SEQ ID | ATGGCGCTGCCAGTCACTGCATTGTTATTGCCTCTGGCC |
| SN66E3.2 | NO: 279 | CTGCTTCTCCATGCGGCAAGGCCAGATATAGTGATGACA |
| (LH)_LIR1 | | CAGTCCCCCGACTCCCTGGCTGTCTCACTGGGAGAACGA |
| (30)_ | | GCGACGATTAGTTGTAAGTCTAGCCAGAGCGTCCTGTAT |
| (IRESL)_H | | TCAAGCAATAACAAGAATTACCTCGCCTGGTATCAGCAA |
| ER2 | | AAGCCGGGACAGCCACCCAAACTGTTGATTTACTGGGCC |
| nucleotide | | AGCACGAGAGAGAGCGGAGTGCCCGACCGCTTCAGCGG |
| Sequence | | ATCCGGGTCAGGCACAGATTTTACCCTGACTATTAGCTC |
| (VR447) | | CCTTCAAGCGGAAGATGTCGCCGTCTACTATTGCCAGCA |
| | | ATATTACGGAACTCCATTCACATTCGGCGGTGGGACCAA |
| | | AGTAGAGATAAAGGGTGGCGGGGGATCCGGCGGTGGCG |
| | | GTAGCGGGGGAGGCGGGTCCCAAGTGCAACTAGTCCAA |
| | | TCAGGTGCCGAAGTCAAGAAACCAGGTGCATCCGTGAA |
| | | AGTGTCTTGCAAAGCCAGTGGCTACACTTTTACTGACTA |
| | | CTATCTGCACTGGGTGCGTCAAGCACCCGGCCAGGGGCT |
| | | TGAATGGATGGGCTGGATTAACCCTTATACTGGAGGGAC |
| | | AAATTACGCTCAGAAGTTCCAGGGACGCGTTACAATGA |
| | | CCCGAGACACCAGCATCAGCACAGCGTACATGGAGTTA |
| | | AGTGGGCTGACTTCCGACGATACCGCCGTGTATTACTGC |
| | | GCTCGGGCAGGGGCCTCTTACTATGATTTTTGGTCCGGT |
| | | TGGGTCTTCGATTACTGGGGGCAGGGAACCCTGGTGACA |
| | | GTGTCCTCAGGCCCCACTTCTACCAGTGGACCGGAAGAT |
| | | CAACCACTTACACCAACGGGCAGCGACCCCCAGTCAGG |
| | | CCTAGGGCGCCACCTGGGTGTGGTCATCGGGATACTGGT |
| | | CGCTGTCATCCTGCTTCTGCTCCTTCTCTTGCTCCTATTC |
| | | CTAATCCTGCGCCACAGGAGACAGGGCAAGCACTGGAC |
| | | CAGCACCCAGCGGAAGGCCGACTTTCAGCACCCTGCCG |
| | | GCGCCGTGGGCCCTGAGCCTACCGACAGGGGCCTGCAG |
| | | TGGAGGAGCTCCCCAGCCGCCGATGCCCAGGAGGAGAA |
| | | TCTGTACGCCGCCGTGAAGCACACCCAGCCAGAGGACG |
| | | GCGTGGAGATGGACACCCGCTCCCCACACGACGAGGAC |
| | | CCACAGGCCGTGACCTACGCCGAGGTGAAGCACAGCCG |
| | | CCCCAGACGCGAGATGGCCAGCCCACCCAGCCCCCTGTC |
| | | CGGCGAGTTCCTGGACACCAAGGACAGGCAGGCCGAGG |
| | | AGGACCGGCAGATGGACACCGAGGCCGCCGCCTCCGAG |
| | | GCCCCCCAGGACGTGACCTACGCCCAGCTGCACTCCCTG |
| | | ACCCTGCGGAGAGAGGCCACCGAGCCCCCACCCAGCCA |
| | | GGAGGGCCCCTCCCCCGCCGTGCCTAGCATCTACGCCAC |
| | | CCTGGCCATCCACTGATAACCCCCCCCCCTAACGTTACT |
| | | GGCCGAAGCCGCTTGGAATAAGGCCGGTGTGCGTTTGTC |
| | | TATATGTTATTTTCCACCATATTGCCGTCTTTTGGCAATG |
| | | TGAGGGCCCGGAAACCTGGCCCTGTCTTCTTGACGAGCA |
| | | TTCCTAGGGGTCTTTCCCCTCTCGCCAAAGGAATGCAAG |
| | | GTCTGTTGAATGTCGTGAAGGAAGCAGTTCCTCTGGAAG |
| | | CTTCTTGAAGACAAACAACGTCTGTAGCGACCCTTTGCA |
| | | GGCAGCGGAACCCCCCACCTGGCGACAGGTGCCTCTGC |
| | | GGCCAAAAGCCACGTGTATAAGATACACCTGCAAAGGC |
| | | GGCACAACCCCAGTGCCACGTTGTGAGTTGGATAGTTGT |
| | | GGAAAGAGTCAAATGGCTCTCCTCAAGCGTATTCAACA |
| | | AGGGGCTGAAGGATGCCCAGAAGGTACCCCATTGTATG |
| | | GGATCTGATCTGGGGCCTCGGTGCACATGCTTTACATGT |
| | | GTTTAGTCGAGGTTAAAAAAACGTCTAGGCCCCCCGAAC |
| | | CACGGGGACGTGGTTTTCCTTTGAAAAACACGATGATAA |
| | | TATGATGGCGCTGCCAGTCACTGCATTGTTATTGCCTCT |
| | | GGCCCTGCTTCTCCATGCGGCGCGCCCAGACATCCAGAT |
| | | GACCCAATCCCCAAGCAGTCTCTCAGCCAGCGTGGGAG |
| | | ACAGGGTTACAATCACGTGCCGCGCCAGCCAGGACGTC |
| | | AACACCGCTGTGGCTTGGTATCAGCAAAAGCCCGGGAA |
| | | GGCACCAAAGCTGCTTATTTATAGCGCCTCCTTCTTGTAT |
| | | TCTGGAGTGCCATCCAGGTTTTCCGGGTCACGTAGCGGG |
| | | ACTGACTTTACCCTCACCATATCCAGCCTCCAGCCCGAG |
| | | GATTTCGCCACCTATTACTGTCAGCAACACTACACGACT |
| | | CCACCGACTTTTGGACAGGGCACTAAAGTGGAGATTAA |
| | | GGGCAGCACGAGTGGGAGTGGAAAGCCCGGCAGCGGG |
| | | GAGGGGTCTACCAAGGGAGAGGTCCAGCTGGTTGAATC |
| | | CGGAGGCGGGCTTGTGCAACCTGGAGGCTCCCTGAGGC |
| | | TTAGTTGTGCCGCGTCAGGATTCAACATTAAGGATACCT |
| | | ATATTCATTGGGTCCGACAAGCCCCGGGCAAGGGCTTGG |
| | | AGTGGGTGGCCAGAATCTATCCGACCAACGGATATACA |
| | | AGGTACGCCGATTCTGTGAAAGGACGCTTCACCATCAGC |
| | | GCGGACACATCCAAAAACACAGCCTATCTGCAGATGAA |
| | | CTCCCTTCGCGCCGAGGATACAGCCGTGTACTATTGTAG |
| | | TCGGTGGGGAGGCGACGGCTTCTACGCGATGGACTATTG |
| | | GGGACAAGGAACACTGGTGACTGTCAGTAGCACTACGA |
| | | CCCCAGCACCTAGACCTCCCACCCCAGCTCCAACTATAG |
| | | CTTCCCAGCCATTGTCTCTCCGGCCAGAGGCGTGTCGAC |
| | | CAGCCGCTGGAGGGGCCGTTCATACAAGAGGACTCGAT |
| | | TTCGCTTGCGATATCTACATATGGGCCCCTCTTGCCGGG |
| | | ACATGCGGTGTCCTGCTTCTAAGCTTGGTTATTACCCTCT |
| | | ATTGCAAAAGAGGACGAAAGAAACTGCTTTATATATTC |
| | | AAGCAACCTTTCATGCGCCCCGTACAGACCACGCAGGA |
| | | GGAAGATGGGTGTAGCTGTCGCTTCCCTGAGGAAGAGG |
| | | AAGGTGGATGCGAGTTGCGGGTGAAGTTCAGTCGATCC |
| | | GCCGATGCGCCTGCCTATCAGCAAGGGCAGAACCAGCT |
| | | TTATAACGAGTTAAACCTTGGCCGCCGGGAAGAGTATG |
| | | ACGTGTTGGACAAGCGTCGCGGGAGAGACCCTGAGATG |
| | | GGCGGAAAACCAAGGAGAAAAAATCCACAGGAAGGCTT |
| | | ATATAACGAGTTGCAGAAAGACAAGATGGCCGAGGCAT |
| | | ACTCCGAAATCGGAATGAAGGGCGAGCGACGGCGCGGC |
| | | AAAGGCCACGATGGACTCTATCAGGGCTTAAGCACCGC |
| | | CACCAAAGACACCTACGATGCACTTCATATGCAGGCACT |
| | | CCCACCTAGATGATAA |
| MC0447 | SEQ ID | MALPVTALLLPLALLLHAARPDIVMTQSPDSLAVSLGERAT |
| SN66E3.2 | NO: 280 | ISCKSSQSVLYSSNNKNYLAWYQQKPGQPPKLLIYWASTR |
| (LH)_LIR1 | | ESGVPDRFSGSGSGTDFTLTISSLQAEDVAVYYCQQYYGTP |
| (30)_ | | FTFGGGTKVEIKGGGGSGGGGSGGGGSQVQLVQSGAEVK |
| (IRESL)_H | | KPGASVKVSCKASGYTFTDYYLHWVRQAPGQGLEWMGW |
| ER2 Protein | | INPYTGGTNYAQKFQGRVTMTRDTSISTAYMELSGLTSDD |
| Sequence | | TAVYYCARAGASYYDFWSGWVFDYWGQGTLVTVSSGPT |
| (VR447) | | STSGPEDQPLTPTGSDPQSGLGRHLGVVIGILVAVILLLLLL |
| | | LLLFLILRHRRQGKHWTSTQRKADFQHPAGAVGPEPTDRG |
| | | LQWRSSPAADAQEENLYAAVKHTQPEDGVEMDTRSPHDE |
| | | DPQAVTYAEVKHSRPRREMASPPSPLSGEFLDTKDRQAEE |
| | | DRQMDTEAAASEAPQDVTYAQLHSLTLRREATEPPPSQEG |
| | | PSPAVPSIYATLAIH* |
| | | MALPVTALLLPLALLLHAARPDIQMTQSPSSLSASVGDRVT |
| | | ITCRASQDVNTAVAWYQQKPGKAPKLLIYSASFLYSGVPS |
| | | RFSGSRSGTDFTLTISSLQPEDFATYYCQQHYTTPPTFGQGT |
| | | KVEIKGSTSGSGKPGSGEGSTKGEVQLVESGGGLVQPGGSL |
| | | RLSCAASGFNIKDTYIHWVRQAPGKGLEWVARIYPTNGYT |
| | | RYADSVKGRFTISADTSKNTAYLQMNSLRAEDTAVYYCSR |
| | | WGGDGFYAMDYWGQGTLVTVSSTTTPAPRPPTPAPTIASQ |
| | | PLSLRPEACRPAAGGAVHTRGLDFACDIYIWAPLAGTCGV |
| | | LLLSLVITLYCKRGRKKLLYIFKQPFMRPVQTTQEEDGCSC |
| | | RFPEEEEGGCELRVKFSRSADAPAYQQGQNQLYNELNLGR |
| | | REEYDVLDKRRGRDPEMGGKPRRKNPQEGLYNELQKDK |
| | | MAEAYSEIGMKGERRRGKGHDGLYQGLSTATKDTYDALH |
| | | MQALPPR* |
| MC0449 | SEQ ID | ATGGCGCTGCCAGTCACTGCATTGTTATTGCCTCTGGCC |
| SN66E3.3 | NO: 281 | CTGCTTCTCCATGCGGCAAGGCCAGATATAGTGATGACA |
| (LH)_LIR1 | | CAGTCCCCCGACTCCCTGGCTGTCTCACTGGGAGAACGA |
| (26)_ | | GCGACGATTAGTTGTAAGTCTAGCCAGAGCGTCCTGTAT |
| (IRESL)_H | | TCAAGCAATAACAAGAATTACCTCGCCTGGTATCAGCAA |
| ER2 | | AAGCCGGGACAGCCACCCAAACTGTTGATTTACTGGGCC |
| Nucleotide | | AGCACGAGAGAGAGCGGAGTGCCCGACCGCTTCAGCGG |
| Sequence | | ATCCGGGTCAGGCACAGATTTTACCCTGACTATTAGCTC |
| (VR449) | | CCTTCAAGCGGAAGATGTCGCCGTCTACTATTGCCAGCA |
| | | ATATTACGGAACTCCATTCACATTCGGCGGTGGGACCAA |
| | | AGTAGAGATAAAGGGTGGCGGGGGATCCGGCGGTGGCG |
| | | GTAGCGGGGGAGGCGGGTCCCAAGTGCAACTAGTCCAA |
| | | TCAGGTGCCGAAGTCAAGAAACCAGGTGCATCCGTGAA |
| | | AGTGTCTTGCAAAGCCAGTGGCTACACTTTTACTGACTA |
| | | CTATCTGCACTGGGTGCGTCAAGCACCCGGCCAGGGGCT |
| | | TGAATGGATGGGCTGGATTAACCCTTATACTGGAGGGAC |
| | | AAATTACGCTCAGAAGTTCCAGGGACGCGTTACAATGA |
| | | CCCGAGACACCAGCATCAGCACAGCGTACATGGAGTTA |
| | | AGTAGGCTGAGGTCCGAAGATACCGCCGTGTATTACTGC |
| | | GCTCGGGCAGGGGCCTCTTACTATGATTTTTGGTCCGGT |
| | | TGGGTCTTCGATTACTGGGGGCAGGGAACCCTGGTGACA |
| | | GTGTCCTCAACCAGTGGACCGGAAGATCAACCACTTACA |
| | | CCAACGGGCAGCGACCCCCAGTCAGGCCTAGGGCGCCA |
| | | CCTGGGTGTGGTCATCGGGATACTGGTCGCTGTCATCCT |
| | | GCTTCTGCTCCTTCTCTTGCTCCTATTCCTAATCCTGCGC |
| | | CACAGGAGACAGGGCAAGCACTGGACCAGCACCCAGCG |
| | | GAAGGCCGACTTTCAGCACCCTGCCGGCGCCGTGGGCCC |
| | | TGAGCCTACCGACAGGGGCCTGCAGTGGAGGAGCTCCC |
| | | CAGCCGCCGATGCCCAGGAGGAGAATCTGTACGCCGCC |
| | | GTGAAGCACACCCAGCCAGAGGACGGCGTGGAGATGGA |
| | | CACCCGCTCCCCACACGACGAGGACCCACAGGCCGTGA |
| | | CCTACGCCGAGGTGAAGCACAGCCGCCCCAGACGCGAG |
| | | ATGGCCAGCCCACCCAGCCCCCTGTCCGGCGAGTTCCTG |
| | | GACACCAAGGACAGGCAGGCCGAGGAGGACCGGCAGA |
| | | TGGACACCGAGGCCGCCGCCTCCGAGGCCCCCCAGGAC |
| | | GTGACCTACGCCCAGCTGCACTCCCTGACCCTGCGGAGA |
| | | GAGGCCACCGAGCCCCCACCCAGCCAGGAGGGCCCCTC |
| | | CCCCGCCGTGCCTAGCATCTACGCCACCCTGGCCATCCA |
| | | CTGATAACCCCCCCCCCTAACGTTACTGGCCGAAGCCGC |
| | | TTGGAATAAGGCCGGTGTGCGTTTGTCTATATGTTATTTT |
| | | CCACCATATTGCCGTCTTTTGGCAATGTGAGGGCCCGGA |
| | | AACCTGGCCCTGTCTTCTTGACGAGCATTCCTAGGGGTC |
| | | TTTCCCCTCTCGCCAAAGGAATGCAAGGTCTGTTGAATG |
| | | TCGTGAAGGAAGCAGTTCCTCTGGAAGCTTCTTGAAGAC |
| | | AAACAACGTCTGTAGCGACCCTTTGCAGGCAGCGGAAC |
| | | CCCCCACCTGGCGACAGGTGCCTCTGCGGCCAAAAGCC |
| | | ACGTGTATAAGATACACCTGCAAAGGCGGCACAACCCC |
| | | AGTGCCACGTTGTGAGTTGGATAGTTGTGGAAAGAGTCA |
| | | AATGGCTCTCCTCAAGCGTATTCAACAAGGGGCTGAAG |
| | | GATGCCCAGAAGGTACCCCATTGTATGGGATCTGATCTG |
| | | GGGCCTCGGTGCACATGCTTTACATGTGTTTAGTCGAGG |
| | | TTAAAAAAACGTCTAGGCCCCCCGAACCACGGGGACGT |
| | | GGTTTTCCTTTGAAAAACACGATGATAATATGATGGCGC |
| | | TGCCAGTCACTGCATTGTTATTGCCTCTGGCCCTGCTTCT |
| | | CCATGCGGCGCGCCCAGACATCCAGATGACCCAATCCCC |
| | | AAGCAGTCTCTCAGCCAGCGTGGGAGACAGGGTTACAA |
| | | TCACGTGCCGCGCCAGCCAGGACGTCAACACCGCTGTG |
| | | GCTTGGTATCAGCAAAAGCCCGGGAAGGCACCAAAGCT |
| | | GCTTATTTATAGCGCCTCCTTCTTGTATTCTGGAGTGCCA |
| | | TCCAGGTTTTCCGGGTCACGTAGCGGGACTGACTTTACC |
| | | CTCACCATATCCAGCCTCCAGCCCGAGGATTTCGCCACC |
| | | TATTACTGTCAGCAACACTACACGACTCCACCGACTTTT |
| | | GGACAGGGCACTAAAGTGGAGATTAAGGGCAGCACGAG |
| | | TGGGAGTGGAAAGCCCGGCAGCGGGGAGGGGTCTACCA |
| | | AGGGAGAGGTCCAGCTGGTTGAATCCGGAGGCGGGCTT |
| | | GTGCAACCTGGAGGCTCCCTGAGGCTTAGTTGTGCCGCG |
| | | TCAGGATTCAACATTAAGGATACCTATATTCATTGGGTC |
| | | CGACAAGCCCCGGGCAAGGGCTTGGAGTGGGTGGCCAG |
| | | AATCTATCCGACCAACGGATATACAAGGTACGCCGATTC |
| | | TGTGAAAGGACGCTTCACCATCAGCGCGGACACATCCA |
| | | AAAACACAGCCTATCTGCAGATGAACTCCCTTCGCGCCG |
| | | AGGATACAGCCGTGTACTATTGTAGTCGGTGGGGAGGC |
| | | GACGGCTTCTACGCGATGGACTATTGGGGACAAGGAAC |
| | | ACTGGTGACTGTCAGTAGCACTACGACCCCAGCACCTAG |
| | | ACCTCCCACCCCAGCTCCAACTATAGCTTCCCAGCCATT |
| | | GTCTCTCCGGCCAGAGGCGTGTCGACCAGCCGCTGGAG |
| | | GGGCCGTTCATACAAGAGGACTCGATTTCGCTTGCGATA |
| | | TCTACATATGGGCCCCTCTTGCCGGGACATGCGGTGTCC |
| | | TGCTTCTAAGCTTGGTTATTACCCTCTATTGCAAAAGAG |
| | | GACGAAAGAAACTGCTTTATATATTCAAGCAACCTTTCA |
| | | TGCGCCCCGTACAGACCACGCAGGAGGAAGATGGGTGT |
| | | AGCTGTCGCTTCCCTGAGGAAGAGGAAGGTGGATGCGA |
| | | GTTGCGGGTGAAGTTCAGTCGATCCGCCGATGCGCCTGC |
| | | CTATCAGCAAGGGCAGAACCAGCTTTATAACGAGTTAA |
| | | ACCTTGGCCGCCGGGAAGAGTATGACGTGTTGGACAAG |
| | | CGTCGCGGGAGAGACCCTGAGATGGGCGGAAAACCAAG |
| | | GAGAAAAAATCCACAGGAAGGCTTATATAACGAGTTGC |
| | | AGAAAGACAAGATGGCCGAGGCATACTCCGAAATCGGA |
| | | ATGAAGGGCGAGCGACGGCGCGGCAAAGGCCACGATGG |
| | | ACTCTATCAGGGCTTAAGCACCGCCACCAAAGACACCTA |
| | | CGATGCACTTCATATGCAGGCACTCCCACCTAGATGATA |
| | | A |
| MC0449 | SEQ ID | MALPVTALLLPLALLLHAARPDIVMTQSPDSLAVSLGERAT |
| SN66E3.3 | NO: 282 | ISCKSSQSVLYSSNNKNYLAWYQQKPGQPPKLLIYWASTR |
| (LH)_LIR1 | | ESGVPDRFSGSGSGTDFTLTISSLQAEDVAVYYCQQYYGTP |
| (26)_ | | FTFGGGTKVEIKGGGGSGGGGSGGGGSQVQLVQSGAEVK |
| (IRESL)_H | | KPGASVKVSCKASGYTFTDYYLHWVRQAPGQGLEWMGW |
| ER2 Protein | | INPYTGGTNYAQKFQGRVTMTRDTSISTAYMELSRLRSEDT |
| Sequence | | AVYYCARAGASYYDFWSGWVFDYWGQGTLVTVSSTSGP |
| (VR449) | | EDQPLTPTGSDPQSGLGRHLGVVIGILVAVILLLLLLLLLFLI |
| | | LRHRRQGKHWTSTQRKADFQHPAGAVGPEPTDRGLQWRS |
| | | SPAADAQEENLYAAVKHTQPEDGVEMDTRSPHDEDPQAV |
| | | TYAEVKHSRPRREMASPPSPLSGEFLDTKDRQAEEDRQMD |
| | | TEAAASEAPQDVTYAQLHSLTLRREATEPPPSQEGPSPAVP |
| | | SIYATLAIH* |
| | | MALPVTALLLPLALLLHAARPDIQMTQSPSSLSASVGDRVT |
| | | ITCRASQDVNTAVAWYQQKPGKAPKLLIYSASFLYSGVPS |
| | | RFSGSRSGTDFTLTISSLQPEDFATYYCQQHYTTPPTFGQGT |
| | | KVEIKGSTSGSGKPGSGEGSTKGEVQLVESGGGLVQPGGSL |
| | | RLSCAASGFNIKDTYIHWVRQAPGKGLEWVARIYPTNGYT |
| | | RYADSVKGRFTISADTSKNTAYLQMNSLRAEDTAVYYCSR |
| | | WGGDGFYAMDYWGQGTLVTVSSTTTPAPRPPTPAPTIASQ |
| | | PLSLRPEACRPAAGGAVHTRGLDFACDIYIWAPLAGTCGV |
| | | LLLSLVITLYCKRGRKKLLYIFKQPFMRPVQTTQEEDGCSC |
| | | RFPEEEEGGCELRVKFSRSADAPAYQQGQNQLYNELNLGR |
| | | REEYDVLDKRRGRDPEMGGKPRRKNPQEGLYNELQKDK |
| | | MAEAYSEIGMKGERRRGKGHDGLYQGLSTATKDTYDALH |
| | | MQALPPR* |
| MC0515- | SEQ ID | ATGGCGCTGCCAGTCACTGCATTGTTATTGCCTCTGGCC |
| HzBB7.2(2) | NO: 321 | CTGCTTCTCCATGCGGCAAGGCCACAGGTGCAACTGGTT |
| _LIR1(30)_ | | CAATCTGGTGCTGAGGTGAAAAAGCCCGGCGCATCCGT |
| 2A_HER2 | | GAAAGTGAGCTGTAAGGCATCAGGGTACACCTTCACCA |
| Nucleotide | | GCTATCACATACAATGGGTCCGCCAGGCCCCCGGACAG |
| Sequence | | AGGTTGGAATGGATTGGGTGGATTTACCCGGGTGACGG |
| (VR515) | | CTCAACCCAGTACAATGAGAAGTTCAAGGGCAGGGTGA |
| | | CTATCACACGCGATACCTCCGCGAGCACAGCTTACATGG |
| | | AGTTATCTAGCCTGAGATCCGAAGATACGGCGGTGTATT |
| | | ACTGCGCGCGGGAAGGGACCTACTATGCCATGGACTATT |
| | | GGGGACAAGGGACCCTGGTTACCGTGAGTTCTGGGGGC |
| | | GGGGGTTCCGGGGGAGGGGGATCTGGGGGTGGAGGGAG |
| | | CGATGTGGTAATGACCCAGACACCTTTGTCTTTGAGTGT |
| | | CACCCCCGGACAGCCGGCAAGTATATCCTGTAGATCATC |
| | | CCAATCAATCGTGCACTCCAACGGAAACACATACTTGGA |
| | | ATGGTATCTCCAGAAACCTGGACAGTCCCCACAGTTGCT |
| | | CATCTACAAAGTGTCAAATCGCTTTTCAGGCGTGCCCGA |
| | | TCGTTTCAGCGGCTCAGGCTCCGGGACAGACTTTACATT |
| | | GAAGATTAGCCGCGTAGAGGCAGAGGATGTGGGCGTTT |
| | | ACTATTGTTTTCAAGGGTCACACGTGCCACGCACATTCG |
| | | GCGGCGGTACCAAGGTGGAAATTAAGGGCCCCACTTCT |
| | | ACCAGTGGACCGGAAGATCAACCACTTACACCAACGGG |
| | | CAGCGACCCCCAGTCAGGCCTAGGGCGCCACCTGGGTG |
| | | TGGTCATCGGGATACTGGTCGCTGTCATCCTGCTTCTGCT |
| | | CCTTCTCTTGCTCCTATTCCTAATCCTGCGCCACAGGAGA |
| | | CAGGGCAAGCACTGGACCAGCACCCAGCGGAAGGCCGA |
| | | CTTTCAGCACCCTGCCGGCGCCGTGGGCCCTGAGCCTAC |
| | | CGACAGGGGCCTGCAGTGGAGGAGCTCCCCAGCCGCCG |
| | | ATGCCCAGGAGGAGAATCTGTACGCCGCCGTGAAGCAC |
| | | ACCCAGCCAGAGGACGGCGTGGAGATGGACACCCGCTC |
| | | CCCACACGACGAGGACCCACAGGCCGTGACCTACGCCG |
| | | AGGTGAAGCACAGCCGCCCCAGACGCGAGATGGCCAGC |
| | | CCACCCAGCCCCCTGTCCGGCGAGTTCCTGGACACCAAG |
| | | GACAGGCAGGCCGAGGAGGACCGGCAGATGGACACCG |
| | | AGGCCGCCGCCTCCGAGGCCCCCCAGGACGTGACCTAC |
| | | GCCCAGCTGCACTCCCTGACCCTGCGGAGAGAGGCCAC |
| | | CGAGCCCCCACCCAGCCAGGAGGGCCCCTCCCCCGCCGT |
| | | GCCTAGCATCTACGCCACCCTGGCCATCCACGGATCCGG |
| | | GGAAGGCCGAGGCTCCCTTCTAACATGTGGAGATGTCG |
| | | AGGAAAACCCTGGCCCTATGGCGCTGCCAGTCACTGCAT |
| | | TGTTATTGCCTCTGGCCCTGCTTCTCCATGCGGCGCGCCC |
| | | AGACATCCAGATGACCCAATCCCCAAGCAGTCTCTCAGC |
| | | CAGCGTGGGAGACAGGGTTACAATCACGTGCCGCGCCA |
| | | GCCAGGACGTCAACACCGCTGTGGCTTGGTATCAGCAA |
| | | AAGCCCGGGAAGGCACCAAAGCTGCTTATTTATAGCGC |
| | | CTCCTTCTTGTATTCTGGAGTGCCATCCAGGTTTTCCGGG |
| | | TCACGTAGCGGGACTGACTTTACCCTCACCATATCCAGC |
| | | CTCCAGCCCGAGGATTTCGCCACCTATTACTGTCAGCAA |
| | | CACTACACGACTCCACCGACTTTTGGACAGGGCACTAAA |
| | | GTGGAGATTAAGGGCAGCACGAGTGGGAGTGGAAAGCC |
| | | CGGCAGCGGGGAGGGGTCTACCAAGGGAGAGGTCCAGC |
| | | TGGTTGAATCCGGAGGCGGGCTTGTGCAACCTGGAGGCT |
| | | CCCTGAGGCTTAGTTGTGCCGCGTCAGGATTCAACATTA |
| | | AGGATACCTATATTCATTGGGTCCGACAAGCCCCGGGCA |
| | | AGGGCTTGGAGTGGGTGGCCAGAATCTATCCGACCAAC |
| | | GGATATACAAGGTACGCCGATTCTGTGAAAGGACGCTTC |
| | | ACCATCAGCGCGGACACATCCAAAAACACAGCCTATCT |
| | | GCAGATGAACTCCCTTCGCGCCGAGGATACAGCCGTGTA |
| | | CTATTGTAGTCGGTGGGGAGGCGACGGCTTCTACGCGAT |
| | | GGACTATTGGGGACAAGGAACACTGGTGACTGTCAGTA |
| | | GCACTACGACCCCAGCACCTAGACCTCCCACCCCAGCTC |
| | | CAACTATAGCTTCCCAGCCATTGTCTCTCCGGCCAGAGG |
| | | CGTGTCGACCAGCCGCTGGAGGGGCCGTTCATACAAGA |
| | | GGACTCGATTTCGCTTGCGATATCTACATATGGGCCCCT |
| | | CTTGCCGGGACATGCGGTGTCCTGCTTCTAAGCTTGGTT |
| | | ATTACCCTCTATTGCAAAAGAGGACGAAAGAAACTGCTT |
| | | TATATATTCAAGCAACCTTTCATGCGCCCCGTACAGACC |
| | | ACGCAGGAGGAAGATGGGTGTAGCTGTCGCTTCCCTGA |
| | | GGAAGAGGAAGGTGGATGCGAGTTGCGGGTGAAGTTCA |
| | | GTCGATCCGCCGATGCGCCTGCCTATCAGCAAGGGCAG |
| | | AACCAGCTTTATAACGAGTTAAACCTTGGCCGCCGGGAA |
| | | GAGTATGACGTGTTGGACAAGCGTCGCGGGAGAGACCC |
| | | TGAGATGGGCGGAAAACCAAGGAGAAAAAATCCACAG |
| | | GAAGGCTTATATAACGAGTTGCAGAAAGACAAGATGGC |
| | | CGAGGCATACTCCGAAATCGGAATGAAGGGCGAGCGAC |
| | | GGCGCGGCAAAGGCCACGATGGACTCTATCAGGGCTTA |
| | | AGCACCGCCACCAAAGACACCTACGATGCACTTCATATG |
| | | CAGGCACTCCCACCTAGATGATAA |
| MC0515 | SEQ ID | MALPVTALLLPLALLLHAARPQVQLVQSGAEVKKPGASV |
| HzBB7.2(2) | NO: 322 | KVSCKASGYTFTSYHIQWVRQAPGQRLEWIGWIYPGDGST |
| _LIR1(30)_ | | QYNEKFKGRVTITRDTSASTAYMELSSLRSEDTAVYYCAR |
| 2A_HER2 | | EGTYYAMDYWGQGTLVTVSSGGGGSGGGGSGGGGSDVV |
| Protein | | MTQTPLSLSVTPGQPASISCRSSQSIVHSNGNTYLEWYLQK |
| Sequence | | PGQSPQLLIYKVSNRFSGVPDRFSGSGSGTDFTLKISRVEAE |
| (VR515) | | DVGVYYCFQGSHVPRTFGGGTKVEIKGPTSTSGPEDQPLTP |
| | | TGSDPQSGLGRHLGVVIGILVAVILLLLLLLLLFLILRHRRQ |
| | | GKHWTSTQRKADFQHPAGAVGPEPTDRGLQWRSSPAADA |
| | | QEENLYAAVKHTQPEDGVEMDTRSPHDEDPQAVTYAEVK |
| | | HSRPRREMASPPSPLSGEFLDTKDRQAEEDRQMDTEAAAS |
| | | EAPQDVTYAQLHSLTLRREATEPPPSQEGPSPAVPSIYATLA |
| | | IHGSGEGRGSLLTCGDVEENPGPMALPVTALLLPLALLLHA |
| | | ARPDIQMTQSPSSLSASVGDRVTITCRASQDVNTAVAWYQ |
| | | QKPGKAPKLLIYSASFLYSGVPSRFSGSRSGTDFTLTISSLQP |
| | | EDFATYYCQQHYTTPPTFGQGTKVEIKGSTSGSGKPGSGEG |
| | | STKGEVQLVESGGGLVQPGGSLRLSCAASGFNIKDTYIHW |
| | | VRQAPGKGLEWVARIYPTNGYTRYADSVKGRFTISADTSK |
| | | NTAYLQMNSLRAEDTAVYYCSRWGGDGFYAMDYWGQG |
| | | TLVTVSSTTTPAPRPPTPAPTIASQPLSLRPEACRPAAGGAV |
| | | HTRGLDFACDIYIWAPLAGTCGVLLLSLVITLYCKRGRKKL |
| | | LYIFKQPFMRPVQTTQEEDGCSCRFPEEEEGGCELRVKFSR |
| | | SADAPAYQQGQNQLYNELNLGRREEYDVLDKRRGRDPEM |
| | | GGKPRRKNPQEGLYNELQKDKMAEAYSEIGMKGERRRGK |
| | | GHDGLYQGLSTATKDTYDALHMQALPPR |
| MC0516- | SEQ ID | ATGGCGCTGCCAGTCACTGCATTGTTATTGCCTCTGGCC |
| SN66E3.2 | NO: 323 | CTGCTTCTCCATGCGGCAAGGCCAGATATAGTGATGACA |
| (LH)_LIR1 | | CAGTCCCCCGACTCCCTGGCTGTCTCACTGGGAGAACGA |
| (30)_2A_HE | | GCGACGATTAGTTGTAAGTCTAGCCAGAGCGTCCTGTAT |
| R2 | | TCAAGCAATAACAAGAATTACCTCGCCTGGTATCAGCAA |
| Nucleotide | | AAGCCGGGACAGCCACCCAAACTGTTGATTTACTGGGCC |
| Sequence | | AGCACGAGAGAGAGCGGAGTGCCCGACCGCTTCAGCGG |
| (VR516) | | ATCCGGGTCAGGCACAGATTTTACCCTGACTATTAGCTC |
| | | CCTTCAAGCGGAAGATGTCGCCGTCTACTATTGCCAGCA |
| | | ATATTACGGAACTCCATTCACATTCGGCGGTGGGACCAA |
| | | AGTAGAGATAAAGGGTGGCGGGGGATCCGGCGGTGGCG |
| | | GTAGCGGGGGAGGCGGGTCCCAAGTGCAACTAGTCCAA |
| | | TCAGGTGCCGAAGTCAAGAAACCAGGTGCATCCGTGAA |
| | | AGTGTCTTGCAAAGCCAGTGGCTACACTTTTACTGACTA |
| | | CTATCTGCACTGGGTGCGTCAAGCACCCGGCCAGGGGCT |
| | | TGAATGGATGGGCTGGATTAACCCTTATACTGGAGGGAC |
| | | AAATTACGCTCAGAAGTTCCAGGGACGCGTTACAATGA |
| | | CCCGAGACACCAGCATCAGCACAGCGTACATGGAGTTA |
| | | AGTGGGCTGACTTCCGACGATACCGCCGTGTATTACTGC |
| | | GCTCGGGCAGGGGCCTCTTACTATGATTTTTGGTCCGGT |
| | | TGGGTCTTCGATTACTGGGGGCAGGGAACCCTGGTGACA |
| | | GTGTCCTCAGGCCCCACTTCTACCAGTGGACCGGAAGAT |
| | | CAACCACTTACACCAACGGGCAGCGACCCCCAGTCAGG |
| | | CCTAGGGCGCCACCTGGGTGTGGTCATCGGGATACTGGT |
| | | CGCTGTCATCCTGCTTCTGCTCCTTCTCTTGCTCCTATTC |
| | | CTAATCCTGCGCCACAGGAGACAGGGCAAGCACTGGAC |
| | | CAGCACCCAGCGGAAGGCCGACTTTCAGCACCCTGCCG |
| | | GCGCCGTGGGCCCTGAGCCTACCGACAGGGGCCTGCAG |
| | | TGGAGGAGCTCCCCAGCCGCCGATGCCCAGGAGGAGAA |
| | | TCTGTACGCCGCCGTGAAGCACACCCAGCCAGAGGACG |
| | | GCGTGGAGATGGACACCCGCTCCCCACACGACGAGGAC |
| | | CCACAGGCCGTGACCTACGCCGAGGTGAAGCACAGCCG |
| | | CCCCAGACGCGAGATGGCCAGCCCACCCAGCCCCCTGTC |
| | | CGGCGAGTTCCTGGACACCAAGGACAGGCAGGCCGAGG |
| | | AGGACCGGCAGATGGACACCGAGGCCGCCGCCTCCGAG |
| | | GCCCCCCAGGACGTGACCTACGCCCAGCTGCACTCCCTG |
| | | ACCCTGCGGAGAGAGGCCACCGAGCCCCCACCCAGCCA |
| | | GGAGGGCCCCTCCCCCGCCGTGCCTAGCATCTACGCCAC |
| | | CCTGGCCATCCACGGATCCGGGGAAGGCCGAGGCTCCC |
| | | TTCTAACATGTGGAGATGTCGAGGAAAACCCTGGCCCTA |
| | | TGGCGCTGCCAGTCACTGCATTGTTATTGCCTCTGGCCCT |
| | | GCTTCTCCATGCGGCGCGCCCAGACATCCAGATGACCCA |
| | | ATCCCCAAGCAGTCTCTCAGCCAGCGTGGGAGACAGGG |
| | | TTACAATCACGTGCCGCGCCAGCCAGGACGTCAACACC |
| | | GCTGTGGCTTGGTATCAGCAAAAGCCCGGGAAGGCACC |
| | | AAAGCTGCTTATTTATAGCGCCTCCTTCTTGTATTCTGGA |
| | | GTGCCATCCAGGTTTTCCGGGTCACGTAGCGGGACTGAC |
| | | TTTACCCTCACCATATCCAGCCTCCAGCCCGAGGATTTC |
| | | GCCACCTATTACTGTCAGCAACACTACACGACTCCACCG |
| | | ACTTTTGGACAGGGCACTAAAGTGGAGATTAAGGGCAG |
| | | CACGAGTGGGAGTGGAAAGCCCGGCAGCGGGGAGGGGT |
| | | CTACCAAGGGAGAGGTCCAGCTGGTTGAATCCGGAGGC |
| | | GGGCTTGTGCAACCTGGAGGCTCCCTGAGGCTTAGTTGT |
| | | GCCGCGTCAGGATTCAACATTAAGGATACCTATATTCAT |
| | | TGGGTCCGACAAGCCCCGGGCAAGGGCTTGGAGTGGGT |
| | | GGCCAGAATCTATCCGACCAACGGATATACAAGGTACG |
| | | CCGATTCTGTGAAAGGACGCTTCACCATCAGCGCGGACA |
| | | CATCCAAAAACACAGCCTATCTGCAGATGAACTCCCTTC |
| | | GCGCCGAGGATACAGCCGTGTACTATTGTAGTCGGTGGG |
| | | GAGGCGACGGCTTCTACGCGATGGACTATTGGGGACAA |
| | | GGAACACTGGTGACTGTCAGTAGCACTACGACCCCAGC |
| | | ACCTAGACCTCCCACCCCAGCTCCAACTATAGCTTCCCA |
| | | GCCATTGTCTCTCCGGCCAGAGGCGTGTCGACCAGCCGC |
| | | TGGAGGGGCCGTTCATACAAGAGGACTCGATTTCGCTTG |
| | | CGATATCTACATATGGGCCCCTCTTGCCGGGACATGCGG |
| | | TGTCCTGCTTCTAAGCTTGGTTATTACCCTCTATTGCAAA |
| | | AGAGGACGAAAGAAACTGCTTTATATATTCAAGCAACC |
| | | TTTCATGCGCCCCGTACAGACCACGCAGGAGGAAGATG |
| | | GGTGTAGCTGTCGCTTCCCTGAGGAAGAGGAAGGTGGA |
| | | TGCGAGTTGCGGGTGAAGTTCAGTCGATCCGCCGATGCG |
| | | CCTGCCTATCAGCAAGGGCAGAACCAGCTTTATAACGA |
| | | GTTAAACCTTGGCCGCCGGGAAGAGTATGACGTGTTGG |
| | | ACAAGCGTCGCGGGAGAGACCCTGAGATGGGCGGAAAA |
| | | CCAAGGAGAAAAAATCCACAGGAAGGCTTATATAACGA |
| | | GTTGCAGAAAGACAAGATGGCCGAGGCATACTCCGAAA |
| | | TCGGAATGAAGGGCGAGCGACGGCGCGGCAAAGGCCAC |
| | | GATGGACTCTATCAGGGCTTAAGCACCGCCACCAAAGA |
| | | CACCTACGATGCACTTCATATGCAGGCACTCCCACCTAG |
| | | ATGATAA |
| MC0516- | SEQ ID | MALPVTALLLPLALLLHAARPDIVMTQSPDSLAVSLGERAT |
| SN66E3.2 | NO: 324 | ISCKSSQSVLYSSNNKNYLAWYQQKPGQPPKLLIYWASTR |
| (LH)_LIR1 | | ESGVPDRFSGSGSGTDFTLTISSLQAEDVAVYYCQQYYGTP |
| (30)_2A_HE | | FTFGGGTKVEIKGGGGSGGGGSGGGGSQVQLVQSGAEVK |
| R2 | | KPGASVKVSCKASGYTFTDYYLHWVRQAPGQGLEWMGW |
| Protein | | INPYTGGTNYAQKFQGRVTMTRDTSISTAYMELSGLTSDD |
| Sequence | | TAVYYCARAGASYYDFWSGWVFDYWGQGTLVTVSSGPT |
| (VR516) | | STSGPEDQPLTPTGSDPQSGLGRHLGVVIGILVAVILLLLLL |
| | | LLLFLILRHRRQGKHWTSTQRKADFQHPAGAVGPEPTDRG |
| | | LQWRSSPAADAQEENLYAAVKHTQPEDGVEMDTRSPHDE |
| | | DPQAVTYAEVKHSRPRREMASPPSPLSGEFLDTKDRQAEE |
| | | DRQMDTEAAASEAPQDVTYAQLHSLTLRREATEPPPSQEG |
| | | PSPAVPSIYATLAIHGSGEGRGSLLTCGDVEENPGPMALPV |
| | | TALLLPLALLLHAARPDIQMTQSPSSLSASVGDRVTITCRAS |
| | | QDVNTAVAWYQQKPGKAPKLLIYSASFLYSGVPSRFSGSR |
| | | SGTDFTLTISSLQPEDFATYYCQQHYTTPPTFGQGTKVEIKG |
| | | STSGSGKPGSGEGSTKGEVQLVESGGGLVQPGGSLRLSCA |
| | | ASGFNIKDTYIHWVRQAPGKGLEWVARIYPTNGYTRYADS |
| | | VKGRFTISADTSKNTAYLQMNSLRAEDTAVYYCSRWGGD |
| | | GFYAMDYWGQGTLVTVSSTTTPAPRPPTPAPTIASQPLSLR |
| | | PEACRPAAGGAVHTRGLDFACDIYIWAPLAGTCGVLLLSL |
| | | VITLYCKRGRKKLLYIFKQPFMRPVQTTQEEDGCSCRFPEE |
| | | EEGGCELRVKFSRSADAPAYQQGQNQLYNELNLGRREEY |
| | | DVLDKRRGRDPEMGGKPRRKNPQEGLYNELQKDKMAEA |
| | | YSEIGMKGERRRGKGHDGLYQGLSTATKDTYDALHMQAL |
| | | PPR |
| MC0517- | SEQ ID | ATGGCGCTGCCAGTCACTGCATTGTTATTGCCTCTGGCC |
| SN66E3.3 | NO: 325 | CTGCTTCTCCATGCGGCAAGGCCAGATATAGTGATGACA |
| (LH)_LIR1 | | CAGTCCCCCGACTCCCTGGCTGTCTCACTGGGAGAACGA |
| (26)_2A_HE | | GCGACGATTAGTTGTAAGTCTAGCCAGAGCGTCCTGTAT |
| R2 | | TCAAGCAATAACAAGAATTACCTCGCCTGGTATCAGCAA |
| Nucleotide | | AAGCCGGGACAGCCACCCAAACTGTTGATTTACTGGGCC |
| Sequence | | AGCACGAGAGAGAGCGGAGTGCCCGACCGCTTCAGCGG |
| (VR517) | | ATCCGGGTCAGGCACAGATTTTACCCTGACTATTAGCTC |
| | | CCTTCAAGCGGAAGATGTCGCCGTCTACTATTGCCAGCA |
| | | ATATTACGGAACTCCATTCACATTCGGCGGTGGGACCAA |
| | | AGTAGAGATAAAGGGTGGCGGGGGATCCGGCGGTGGCG |
| | | GTAGCGGGGGAGGCGGGTCCCAAGTGCAACTAGTCCAA |
| | | TCAGGTGCCGAAGTCAAGAAACCAGGTGCATCCGTGAA |
| | | AGTGTCTTGCAAAGCCAGTGGCTACACTTTTACTGACTA |
| | | CTATCTGCACTGGGTGCGTCAAGCACCCGGCCAGGGGCT |
| | | TGAATGGATGGGCTGGATTAACCCTTATACTGGAGGGAC |
| | | AAATTACGCTCAGAAGTTCCAGGGACGCGTTACAATGA |
| | | CCCGAGACACCAGCATCAGCACAGCGTACATGGAGTTA |
| | | AGTAGGCTGAGGTCCGAAGATACCGCCGTGTATTACTGC |
| | | GCTCGGGCAGGGGCCTCTTACTATGATTTTTGGTCCGGT |
| | | TGGGTCTTCGATTACTGGGGGCAGGGAACCCTGGTGACA |
| | | GTGTCCTCAACCAGTGGACCGGAAGATCAACCACTTACA |
| | | CCAACGGGCAGCGACCCCCAGTCAGGCCTAGGGCGCCA |
| | | CCTGGGTGTGGTCATCGGGATACTGGTCGCTGTCATCCT |
| | | GCTTCTGCTCCTTCTCTTGCTCCTATTCCTAATCCTGCGC |
| | | CACAGGAGACAGGGCAAGCACTGGACCAGCACCCAGCG |
| | | GAAGGCCGACTTTCAGCACCCTGCCGGCGCCGTGGGCCC |
| | | TGAGCCTACCGACAGGGGCCTGCAGTGGAGGAGCTCCC |
| | | CAGCCGCCGATGCCCAGGAGGAGAATCTGTACGCCGCC |
| | | GTGAAGCACACCCAGCCAGAGGACGGCGTGGAGATGGA |
| | | CACCCGCTCCCCACACGACGAGGACCCACAGGCCGTGA |
| | | CCTACGCCGAGGTGAAGCACAGCCGCCCCAGACGCGAG |
| | | ATGGCCAGCCCACCCAGCCCCCTGTCCGGCGAGTTCCTG |
| | | GACACCAAGGACAGGCAGGCCGAGGAGGACCGGCAGA |
| | | TGGACACCGAGGCCGCCGCCTCCGAGGCCCCCCAGGAC |
| | | GTGACCTACGCCCAGCTGCACTCCCTGACCCTGCGGAGA |
| | | GAGGCCACCGAGCCCCCACCCAGCCAGGAGGGCCCCTC |
| | | CCCCGCCGTGCCTAGCATCTACGCCACCCTGGCCATCCA |
| | | CGGATCCGGGGAAGGCCGAGGCTCCCTTCTAACATGTG |
| | | GAGATGTCGAGGAAAACCCTGGCCCTATGGCGCTGCCA |
| | | GTCACTGCATTGTTATTGCCTCTGGCCCTGCTTCTCCATG |
| | | CGGCGCGCCCAGACATCCAGATGACCCAATCCCCAAGC |
| | | AGTCTCTCAGCCAGCGTGGGAGACAGGGTTACAATCAC |
| | | GTGCCGCGCCAGCCAGGACGTCAACACCGCTGTGGCTTG |
| | | GTATCAGCAAAAGCCCGGGAAGGCACCAAAGCTGCTTA |
| | | TTTATAGCGCCTCCTTCTTGTATTCTGGAGTGCCATCCAG |
| | | GTTTTCCGGGTCACGTAGCGGGACTGACTTTACCCTCAC |
| | | CATATCCAGCCTCCAGCCCGAGGATTTCGCCACCTATTA |
| | | CTGTCAGCAACACTACACGACTCCACCGACTTTTGGACA |
| | | GGGCACTAAAGTGGAGATTAAGGGCAGCACGAGTGGGA |
| | | GTGGAAAGCCCGGCAGCGGGGAGGGGTCTACCAAGGGA |
| | | GAGGTCCAGCTGGTTGAATCCGGAGGCGGGCTTGTGCA |
| | | ACCTGGAGGCTCCCTGAGGCTTAGTTGTGCCGCGTCAGG |
| | | ATTCAACATTAAGGATACCTATATTCATTGGGTCCGACA |
| | | AGCCCCGGGCAAGGGCTTGGAGTGGGTGGCCAGAATCT |
| | | ATCCGACCAACGGATATACAAGGTACGCCGATTCTGTGA |
| | | AAGGACGCTTCACCATCAGCGCGGACACATCCAAAAAC |
| | | ACAGCCTATCTGCAGATGAACTCCCTTCGCGCCGAGGAT |
| | | ACAGCCGTGTACTATTGTAGTCGGTGGGGAGGCGACGG |
| | | CTTCTACGCGATGGACTATTGGGGACAAGGAACACTGGT |
| | | GACTGTCAGTAGCACTACGACCCCAGCACCTAGACCTCC |
| | | CACCCCAGCTCCAACTATAGCTTCCCAGCCATTGTCTCT |
| | | CCGGCCAGAGGCGTGTCGACCAGCCGCTGGAGGGGCCG |
| | | TTCATACAAGAGGACTCGATTTCGCTTGCGATATCTACA |
| | | TATGGGCCCCTCTTGCCGGGACATGCGGTGTCCTGCTTC |
| | | TAAGCTTGGTTATTACCCTCTATTGCAAAAGAGGACGAA |
| | | AGAAACTGCTTTATATATTCAAGCAACCTTTCATGCGCC |
| | | CCGTACAGACCACGCAGGAGGAAGATGGGTGTAGCTGT |
| | | CGCTTCCCTGAGGAAGAGGAAGGTGGATGCGAGTTGCG |
| | | GGTGAAGTTCAGTCGATCCGCCGATGCGCCTGCCTATCA |
| | | GCAAGGGCAGAACCAGCTTTATAACGAGTTAAACCTTG |
| | | GCCGCCGGGAAGAGTATGACGTGTTGGACAAGCGTCGC |
| | | GGGAGAGACCCTGAGATGGGCGGAAAACCAAGGAGAA |
| | | AAAATCCACAGGAAGGCTTATATAACGAGTTGCAGAAA |
| | | GACAAGATGGCCGAGGCATACTCCGAAATCGGAATGAA |
| | | GGGCGAGCGACGGCGCGGCAAAGGCCACGATGGACTCT |
| | | ATCAGGGCTTAAGCACCGCCACCAAAGACACCTACGAT |
| | | GCACTTCATATGCAGGCACTCCCACCTAGATGATAA |
| MC0517- | SEQ ID | MALPVTALLLPLALLLHAARPDIVMTQSPDSLAVSLGERAT |
| SN66E3.3 | NO: 326 | ISCKSSQSVLYSSNNKNYLAWYQQKPGQPPKLLIYWASTR |
| (LH)_LIR1 | | ESGVPDRFSGSGSGTDFTLTISSLQAEDVAVYYCQQYYGTP |
| (26)_2A_HE | | FTFGGGTKVEIKGGGGSGGGGSGGGGSQVQLVQSGAEVK |
| R2 Protein | | KPGASVKVSCKASGYTFTDYYLHWVRQAPGQGLEWMGW |
| Sequence | | INPYTGGTNYAQKFQGRVTMTRDTSISTAYMELSRLRSEDT |
| (VR517) | | AVYYCARAGASYYDFWSGWVFDYWGQGTLVTVSSTSGP |
| | | EDQPLTPTGSDPQSGLGRHLGVVIGILVAVILLLLLLLLLFLI |
| | | LRHRRQGKHWTSTQRKADFQHPAGAVGPEPTDRGLQWRS |
| | | SPAADAQEENLYAAVKHTQPEDGVEMDTRSPHDEDPQAV |
| | | TYAEVKHSRPRREMASPPSPLSGEFLDTKDRQAEEDRQMD |
| | | TEAAASEAPQDVTYAQLHSLTLRREATEPPPSQEGPSPAVP |
| | | SIYATLAIHGSGEGRGSLLTCGDVEENPGPMALPVTALLLP |
| | | LALLLHAARPDIQMTQSPSSLSASVGDRVTITCRASQDVNT |
| | | AVAWYQQKPGKAPKLLIYSASFLYSGVPSRFSGSRSGTDFT |
| | | LTISSLQPEDFATYYCQQHYTTPPTFGQGTKVEIKGSTSGSG |
| | | KPGSGEGSTKGEVQLVESGGGLVQPGGSLRLSCAASGFNIK |
| | | DTYIHWVRQAPGKGLEWVARIYPTNGYTRYADSVKGRFTI |
| | | SADTSKNTAYLQMNSLRAEDTAVYYCSRWGGDGFYAMD |
| | | YWGQGTLVTVSSTTTPAPRPPTPAPTIASQPLSLRPEACRPA |
| | | AGGAVHTRGLDFACDIYIWAPLAGTCGVLLLSLVITLYCK |
| | | RGRKKLLYIFKQPFMRPVQTTQEEDGCSCRFPEEEEGGCEL |
| | | RVKFSRSADAPAYQQGQNQLYNELNLGRREEYDVLDKRR |
| | | GRDPEMGGKPRRKNPQEGLYNELQKDKMAEAYSEIGMKG |
| | | ERRRGKGHDGLYQGLSTATKDTYDALHMQALPPR |

ii. Bicistronic iCAR Portion

In some embodiments, the bicistronic iCAR portions described below can be included as part of monocistronic iCAR constructs for use in co-transduction methods along with a described monocistronic aCAR construct.

1. iCAR Portion: scFv Component

In some embodiments, the bicistronic construct comprises an iCAR portion comprising a single chain variable fragment (scFv) component. In some embodiments, the iCAR portion comprises a single chain variable fragment (scFv) component. In some embodiments, the scFv targets an HLA antigen. In some embodiments, the HLA antigen is selected from the group consisting of HLA-A2, HLA-A3, HLA-A, HLA-B, HLA-C, HLA-G, HLA-E, HLA-F, HLA-DPA1, HLA-DQA1, HLA-DQB1, HLA-DQB2, HLA-DRB1, and HLA-DRB5. In some embodiments, the iCAR comprises an scFv. In some embodiments, the scFv is selected from the group consisting of BB7.2, 3PF12, 3PF12/C4, 3PF12/F12, 3PF12/B11, W6/32, BBM.1, SN66E3.1, SN66E3.2, SN66E.3, Ha5C2.A2, MWB1, MWB1-mod, Hz.BB7.2VH1-69_A18VK, Hz.BB7.2VH1-69 (27,30)_A18, HzBB7.2VH1-69 (27,30,48) A18, Hz.BB7.2 VH1-69 (27,30,67)_A18, Hz.BB7.2 VH1-69 (27,30,69)_A18, Hz.BB7.2 VH1-69 (27,30,67,69)_A18, Hz.BB7.2 VH1-3_A18, Hz.BB7.2 VH1-3(48)_A18, Hz.BB7.2 VH1-3(67)_A18, Hz.BB7.2 VH1-3(69)_A18, Hz.BB7.2 VH1-3(71)_A18, Hz.BB7.2 VH1-3(73)_A18, and MWB1.2. In some embodiments, the scFv has the VL and VH sequences of BB7.2 (SEQ ID NOs: 37 and 38). In some embodiments, the scFv has the VL and VH sequences of 3PF12/C4 (SEQ ID NOs: 39 and 40). In some embodiments, the scFv has the VL and VH sequences of 3PF12/F12 (SEQ ID NOs: 41 and 42). In some embodiments, the scFv has the VL and VH sequences of 3PF12/B11 (SEQ ID NOs: 43 and 44). In some embodiments, the scFv has the VL and VH sequences of W6/32 (SEQ ID NOs: 45 and 46). In some embodiments, the scFv has the VL and VH sequences of BBM.1 (SEQ ID NOs: 47 and 48). In some embodiments, the scFv has the VL and VH sequences of SN66E3 (SEQ ID NOs: 49 and 50). In some embodiments, the scFv has the VL and VH sequences of Ha5C2.A2 (SEQ ID NOs: 51 and 52). In some embodiments, the scFv has the VL and VH sequences of MWB1 (SEQ ID NOs: 53 and 54). In some embodiments, the scFv has the VL and VH sequences of MWB1-mod (SEQ ID NOs: 55 and 56). In some embodiments, the scFv has the VL and VH sequences of Hz.BB7.2 VH1-69_A18VK (SEQ ID NOs: 57 and 58). In some embodiments, the scFv has the VL and VH sequences of Hz.BB7.2 VH1-69 (27,30)_A18 (SEQ ID NOs: 59 and 60). In some embodiments, the scFv has the VL and VH sequences of Hz.BB7.2 VH1-69 (27,30,48)_A18 (SEQ ID NOs: 61 and 62). In some embodiments, the scFv has the VL and VH sequences of Hz.BB7.2 VH1-69 (27,30,67)_A18 (SEQ ID NOs: 63 and 64). In some embodiments, the scFv has the VL and VH sequences of Hz.BB7.2 VH1-69 (27,30,69)_A18 (SEQ ID NOs: 65 and 66). In some embodiments, the scFv has the VL and VH sequences of Hz.BB7.2 VH1-69 (27,30,67,69)_A18 (SEQ ID NOs: 67 and 68). In some embodiments, the scFv has the VL and VH sequences of Hz.BB7.2 VH1-3_A18 (SEQ ID NOs: 69 and 70). In some embodiments, the scFv has the VL and VH sequences of Hz.BB7.2 VH1-3(48)_A18 (SEQ ID NOs: 71 and 72). In some embodiments, the scFv has the VL and VH sequences of Hz.BB7.2 VH1-3(67)_A18 (SEQ ID NOs: 73 and 74). In some embodiments, the scFv has the VL and VH sequences of Hz.BB7.2 VH1-3(69)_A18 (SEQ ID NOs: 75 and 76). In some embodiments, the scFv has the VL and VH sequences of Hz.BB7.2 VH1-3(71)_A18 (SEQ ID NOs: 77 and 78). In some embodiments, the scFv has the VL and VH sequences of Hz.BB7.2 VH1-3(73)_A18 (SEQ ID NOs: 79 and 80). In some embodiments, the scFv has the VL and VH sequences of MWB1.2 (SEQ ID NOs: 163 and 164). In some embodiments, the scFv has the VL and VH sequences of SN66E3.2 (SEQ ID NOs: 165 and 166). In some embodiments, the scFv has the VL and VH sequences of SN66E3.3 (SEQ ID NOs: 283 and 284) In some embodiments, the scFv is BB7.2 (SEQ ID NO:167). In some embodiments, the scFv is 3PF12 (SEQ ID NO:168). In some embodiments, the scFv is SN66E3.1 (SEQ ID NO:169). In some embodiments, the scFv is SN66E3.2 (SEQ ID NO:285). In some embodiments, the scFv is SN66E3.3 (SEQ ID NO:286). In some embodiments, the scFv is Hz BB7.2.1 (SEQ ID NO:287). In some embodiments, the scFv is HzBB7.2.2 (SEQ ID NO:288). In some embodiments, the scFv is MWB1.1 (SEQ ID NO:273). In some embodiments, the scFv is MWB1.2 (SEQ ID NO:274). In some embodiments, the scFv is 3PF12/C4. In some embodiments, the scFv is 3PF12/F12. In some embodiments, the scFv is 3PF12/B11. In some embodiments, the scFv is W6/32. In some embodiments, the scFv is BBM.1. In some embodiments, the scFv is Ha5C2.A2. In some embodiments, the scFv is MWB1. In some embodiments, the scFv is MWB1-mod. In some embodiments, the scFv is BB7.2. In some embodiments, the scFv is 3PF12. In some embodiments, the scFv is SN66E3.1. In some embodiments, the scFv is SN66E3.2. In some embodiments, the scFv is SN66E3.3. In some embodiments, the scFv is Hz BB7.2.1. In some embodiments, the scFv is HzBB7.2.2. In some embodiments, the scFv is MWB1.1. In some embodiments, the scFv is MWB1.2. In some embodiments, the scFv is Hz.BB7.2 VH1-69_A18VK. In some embodiments, the scFv is Hz.BB7.2 VH1-69 (27,30)_A18. In some embodiments, the scFv is Hz.BB7.2 VH1-69 (27,30,48)_A18. In some embodiments, the scFv is Hz.BB7.2 VH1-69 (27, 30, 67)_A18. In some embodiments, the scFv is Hz.BB7.2 VH1-69 (27, 30, 69)_A18. In some embodiments, the scFv is Hz.BB7.2 VH1-69 (27, 30, 67, 69)_A18. In some embodiments, the scFv is Hz.BB7.2VH1-3_A18. In some embodiments, the scFv is Hz.BB7.2 VH1-3(48)_A18. In some embodiments, the scFv is Hz.BB7.2-3(67)_A18. In some embodiments, the scFv is Hz.BB7.2 VH1-3(69)_A18. In some embodiments, the scFv is Hz.BB7.2 VH1-3(71)_A18. In some embodiments, the scFv is Hz.BB7.2 VH1-3(73)_A18. In some embodiments, the scFv is MWB1.2. In some embodiments, the scFv is SN66E3.2. In some embodiments, the scFv is MWB1.1. In some embodiments, the scFv is MWB1.2. In some embodiments, the scFv comprises Hz.BB7.2 heavy chain Hz.BB7.2VH1-69. In some embodiments, the scFv comprises Hz.BB7.2 Heavy chain Hz.BB7.2VH1-69(H27Y, H30S. In some embodiments, the scFv comprises Hz.BB7.2 heavy chain HZ.BB7.2VH1-69(H27Y, H30S, H48I). In some embodiments, the scFv comprises Hz.BB7.2 Heavy chain Hz.BB7.2VH1-69(H27Y, H30S, H67T). In some embodiments, the scFv comprises Hz. BB7.2 Heavy chain Hz.BB7.2VH1-69 (H27Y, H30S, H69L). In some embodiments, the scFv comprises Hz.BB7.2 Heavy Chain HZ.BB7.2VH1-69 (H27Y, H30S, VH67T, H69L). In some embodiments, the scFv comprises Hz.BB7.2 Heavy Chain Hz.BB7.2 VH1-3. In some embodiments, the scFv comprises Hz.BB7.2 Heavy Chain Hz.BB7.2 VH1-3 (H48I). In some embodiments, the scFv comprises Hz.BB7.2 Heavy Chain VH1-3 (H67T). In some embodiments, the scFv comprises Hz.BB7.2 Heavy Chain Hz.BB7.2 VH1-3 (H69L). In some embodiments, the scFv comprises Hz.BB7.2 Heavy Chain Hz.BB7.2 VH1-3 (H71A). In some embodiments, the scFv comprises Hz.BB7.2 Heavy Chain Hz.BB7.2 VH1-3 (H73A). In some embodiments, the scFv comprises Hz.BB7.2 Light chain VKA18. The 6 CDR sequences for the variable heavy and variable light chains are shown in bold and underline in Table 2 for each sequence, also referred to as vhCDR1, vhCDR2, vhCDR3, vlCDR1, vlCDR2, and vlCDR3. In some embodiments, the iCAR comprises the 6 CDR sequences for the variable heavy and variable light chains as show in bold and underline in Table 2 for each sequence, also referred to as vhCDR1, vhCDR2, vhCDR3, vlCDR1, vlCDR2, and vlCDR3. In some embodiments, the iCAR comprises the 6 CDR sequences for the variable heavy and variable light chains as show in bold and underline in Table 2 for each sequence, also referred to as vhCDR1, vhCDR2, vhCDR3, vlCDR1, vlCDR2, and vlCDR3, wherein each CDR individually optionally comprises one more substitutions. In some embodiments, the iCAR comprises the 6 CDR sequences for the variable heavy and variable light chains as show in bold and underline in Table 2 for each sequence, also referred to as vhCDR1, vhCDR2, vhCDR3, vlCDR1, vlCDR2, and vlCDR3, wherein each CDR individually optionally comprises 1, 2, and/or 3 substitutions. In some embodiments, the iCAR comprises the 6 CDR sequences for the variable heavy and variable light chains as show in bold and underline in Table 2 for each sequence, also referred to as vhCDR1, vhCDR2, vhCDR3, vlCDR1, vlCDR2, and vlCDR3, wherein each CDR individually comprises one more substitutions. In some embodiments, the iCAR comprises the 6 CDR sequences for the variable heavy and variable light chains as show in bold and underline in Table 2 for each sequence, also referred to as vhCDR1, vhCDR2, vhCDR3, vlCDR1, vlCDR2, and vlCDR3, wherein each CDR individually comprises 1, 2, and/or 3 substitutions.

TABLE 2
iCAR vh, vl, and scFv sequences

| Sequence | SEQ ID | |
|---|---|---|
| Information | NO | Amino acid sequence |
| BB7.2 variable | 37 | DVLMTQTPLSLPVSLGDQVSISCRSSQSIVHSNGNTYLE |
| light chain | | WYLQKPGQSPKLLIYKVSNRFSGVPDRFSGSGSGTDFT |
| | | LKISRVEAEDLGVYYCFQGSHVPRTFGGGTKLEIK |
| BB7.2 variable | 38 | QVQLQQSGPELVKPGASVKMSCKASGYTFTSYHIQWV |
| heavy chain | | KQRPGQGLEWIGWIYPGDGSTQYNEKFKGKTTLTAD |
| | | KSSSTAYMLLSSLTSEDSAIYFCAREGTYYAMDYWGQ |
| | | GTSVTVSS |
| 3PF12/C4 | 39 | DIVMTQSPSFLSASVGDRVTITCRASHGINNYLAWYQQ |
| variable light | | KPGKAPKLLIYAASTLQSGVPSRFSGSGSGTEFTLTISSL |
| chain | | QPEDFATYYCQQYDSYPPTFGRGTKVEIK |
| 3PF12/C4 | 40 | QVQLVQSGGGVVQPGGSLRVSCAASGVTLSDYGMHW |
| variable heavy | | VRQAPGKGLEWMAFIRNDGSDKYYADSVKGRFTISRD |
| chain | | NSKKTVSLQMSSLRAEDTAVYYCAKNGESGPLDYWY |
| | | FDLWGRGTLVTVSS |
| 3PF12/F12 | 41 | DVVMTQSPSSLSASVGDRVTITCQASQDISNYLNWYQQ |
| variable light | | KPGKAPKLLIYDASNLETGVPSRFSGSGSGTDFTFTISSL |
| chain | | QPEDFATYYCQQYSSFPLTFGGGTKVDIK |
| 3PF12/F12 | 42 | QVQLVQSGGGVVQPGGSLRVSCAASGVTLSDYGMHW |
| variable heavy | | VRQAPGKGLEWMAFIRNDGSDKYYADSVKGRFTISRD |
| chain | | NSKKTVSLQMSSLRAEDTAVYYCAKNGESGPLDYWY |
| | | FDLWGRGTLVTVSS |
| 3PF12/B11 | 43 | DVVMTQSPSSLSASVGDRVTITCQASQDISNYLNWYQQ |
| variable light | | KPGKAPKLLIYDASNLETGVPSRFSGSGSGTDFTFTISSL |
| chain | | QPEDIATYYCQQYDNLPPTFGGGTKLEIV |
| 3PF12/B11 | 44 | QVQLVQSGGGVVQPGGSLRVSCAASGVTLSDYGMHW |
| variable heavy | | VRQAPGKGLEWMAFIRNDGSDKYYADSVKGRFTISRD |
| chain | | NSKKTVSLQMSSLRAEDTAVYYCAKNGESGPLDYWY |
| | | FDLWGRGTLVTVSS |
| W6/32 variable | 45 | SIVMTQTPKFLLVSAGDRVTITCKASQSVSNDVAWYQQ |
| light chain | | KPGQSPKLLIYYASNRYTGVPDRFTGSGYGTDFTFTIST |
| | | VQAEDLAVYFCQQDYSSPPWTFGGGTKLEIR |
| W6/32 variable | 46 | QVQLKQSGPGLVQPSQSLSLTCTVSGFSLTSYGVHWVR |
| heavy chain | | QPPGKGLEWLGVIWSGGSTDYNAAFISRLSIRKDNSKS |
| | | QVFFKMNSLQADDTAIYYCARTFTTSTSAWFAYWGQ |
| | | GTLVTVSA |
| BBM.1 variable | 47 | DIQMTQSPASQSASLGESVTITCLASQTIGTWLAWYQQ |
| light chain | | KPGKSPQLLIYAATSLADGVPSRFSGSGSGTKFSLKIRTL |
| | | QAEDFVSYYCQQLYSKPYTFGGGTKLEIK |
| BBM.1 variable | 48 | EVQLQQSGAELVKPGASVKLSCTPSGFNVKDTYIHWV |
| heavy chain | | KQRPKQGLEWIGRIDPSDGDIKYDPKFQGKATITADTS |
| | | SNTVSLQLSSLTSEDTAVYYCARWFGDYGAMNYWGQ |
| | | GTSVTVSS |
| SN66E3.1 | 49 | DIVMTQSPDSLAVSLGERATISCKSSQSVLYSSNNKNYL |
| variable light | | AWYQQKLGQPPKLLIYWASTRESGVPDRFSGSGSGTNF |
| chain | | TLTISSLQAENVAVYYCQQYYGTPFTFGGGTKVEIK |
| SN66E3.1 | 50 | QVQLVQSGAEVKKPGASVKVSCKASGYTFTDYYLHW |
| variable heavy | | VRQAPGQGLEWMGWINPYTGGTNYAQKFQGRVTMT |
| chain | | RDASISTVYMELSGLTSDDTAVHFCARAGASYYDFWS |
| | | GWVFDYWGQGTLVTVSS |
| Ha5C2.A2 | 51 | DIQMTQSPSSLSASVGDRVTITCRASQSISTYLNWYQQK |
| variable light | | PGKAPKLLIYAASSLQSGVPSRESGSGSGTDFTLTISSLQ |
| chain | | PEDFATYQCQQSYSTPFTFGGGTKVEIK |
| Ha5C2.A2 | 52 | QVQLQESGPGLVKPSETLSLTCTVSGGSISSYYWSWIRQ |
| variable heavy | | PAGKGLEWIGRIYISGGTNYNPSLKSRVTMSVDTSKNQ |
| chain | | VSLKLSSVTAADTAVYYCARDILGGVSGWSHYGMDV |
| | | WGQGTTVTVSS |
| MWB 1 variable | 53 | QSALTQPPSASGSPGQSVTISCTGTSSDVGGYKYVSWY |
| light chain | | QHHPDKAPKLMIYEVNKRPSGVPDRFSGSKSDNTASLT |
| | | VSGLQAEDEADYYCSSYAGSNNWVFGGGTKLTVL |
| MWB1 variable | 54 | QVQLVESGGGVVQPGGSLRLSCAASGFTFSTYGMHWV |
| heavy chain | | RQAPGKGLEWAASVSYDGSNKYYADSGQGRFTISRDT |
| | | SMNSLYLQVNSLRDETAVYYCAIGIYGAYSFDYWGQG |
| | | TLVTVSS |
| MWB1.1 | 55 | QSALTQPPSASGSPGQSVTISCTGTSSDVGGYKYVSWY |
| (MWB1.1) | | QHHPDKAPKLMIYEVNKRPSGVPDRFSGSKSDNTASLT |
| variable light | | VSGLQAEDEADYYCSSYAGSNNWVFGGGTKLTVL |
| chain | | |
| MWB1.1 (MWB11) | 56 | QVQLVESGGGVVQPGGSLRLSCAASGFTFSTYGMHWV |
| variable heavy | | RQAPGKGLEWVASISYDGSNKYYADSGQGRFTISRDTS |
| chain | | KNSLYLQMNSLRAEDTAVYYCAIGIYGAYSFDYWGQG |
| | | TLVTVSS |
| Hz.BB7.2 | 57 | DVVMTQTPLSLSVTPGQPASISCRSSQSIVHSNGNTYLE |
| A18VK variable | | WYLQKPGQSPQLLIYKVSNRFSGVPDRFSGSGSGTDFT |
| light chain | | LKISRVEAEDVGVYYCFQGSHVPRTFGGGTKVEIK |
| Hz. BB7.2 VH1- | 58 | QVQLVQSGAEVKKPGSSVKVSCKASGGTFSSYHIQWV |
| 69 variable heavy | | RQAPGQGLEWMGWIYPGDGSTQYNEKFKGRVTITAD |
| chain | | KSTSTAYMELSSLRSEDTAVYYCAREGTYYAMDYWG |
| | | QGTLVTVSS |
| Hz.BB7.2 VH1- | 59 | DVVMTQTPLSLSVTPGQPASISCRSSQSIVHSNGNTYLE |
| 69 (27, 30) | | WYLQKPGQSPQLLIYKVSNRFSGVPDRFSGSGSGTDFT |
| variable light | | LKISRVEAEDVGVYYCFQGSHVPRTFGGGTKVEIK |
| chain | | |
| Hz.BB7.2 Heavy | 60 | QVQLVQSGAEVKKPGSSVKVSCKASGYTFTSYHIQWV |
| chain VH1-69 | | RQAPGQGLEWMGWIYPGDGSTQYNEKFKGRVTITAD |
| (H27Y, H30S) | | KSTSTAYMELSSLRSEDTAVYYCAREGTYYAMDYWG |
| | | QGTLVTVSS |
| HZ.BB7.2VH1- | 61 | DVVMTQTPLSLSVTPGQPASISCRSSQSIVHSNGNTYLE |
| 69 (27, 30, 48)_ | | WYLQKPGQSPQLLIYKVSNRFSGVPDRFSGSGSGTDFT |
| A18 variable | | LKISRVEAEDVGVYYCFQGSHVPRTFGGGTKVEIK |
| light chain | | |
| Hz.BB7.2 heavy | 62 | QVQLVQSGAEVKKPGSSVKVSCKASGYTFTSYHIQWV |
| chain VH1-69 | | RQAPGQGLEWIGWIYPGDGSTQYNEKFKGRVTITADK |
| (H27Y, H30S, | | STSTAYMELSSLRSEDTAVYYCAREGTYYAMDYWGQ |
| H48I)) | | GTLVTVSS |
| Hz.BB7.2 VH1-69 | 63 | DVVMTQTPLSLSVTPGQPASISCRSSQSIVHSNGNTYLE |
| (27, 30, 67)_A18 | | WYLQKPGQSPQLLIYKVSNRFSGVPDRFSGSGSGTDFT |
| variable light | | LKISRVEAEDVGVYYCFQGSHVPRTFGGGTKVEIK |
| chain | | |
| Hz.BB7.2 Heavy | 64 | QVQLVQSGAEVKKPGSSVKVSCKASGYTFTSYHIQWV |
| chain VH1-69 | | RQAPGQGLEWMGWIYPGDGSTQYNEKFKGRTTITAD |
| (H27Y, H30S, | | KSTSTAYMELSSLRSEDTAVYYCAREGTYYAMDYWG |
| H67T)) | | QGTLVTVSS |
| HZ.BB7.2VH1-69 | 65 | DVVMTQTPLSLSVTPGQPASISCRSSQSIVHSNGNTYLE |
| (27, 30, 69) | | WYLQKPGQSPQLLIYKVSNRFSGVPDRFSGSGSGTDFT |
| A18 variable | | LKISRVEAEDVGVYYCFQGSHVPRTFGGGTKVEIK |
| light chain | | |
| Hz. BB7.2 Heavy | 66 | QVQLVQSGAEVKKPGSSVKVSCKASGYTFTSYHIQWV |
| chain VH1-69 | | RQAPGQGLEWMGWIYPGDGSTQYNEKFKGRVTLTA |
| (H27Y, H30S, | | DKSTSTAYMELSSLRSEDTAVYYCAREGTYYAMDYW |
| H69L)) | | GQGTLVTVSS |
| Hz.BB7.2 VH1- | 67 | DVVMTQTPLSLSVTPGQPASISCRSSQSIVHSNGNTYLE |
| 69 | | WYLQKPGQSPQLLIYKVSNRFSGVPDRFSGSGSGTDFT |
| (27, 30, 67, 69)_ | | LKISRVEAEDVGVYYCFQGSHVPRTFGGGTKVEIK |
| A18 variable light | | |
| chain | | |
| Hz.BB7.2 Heavy | 68 | QVQLVQSGAEVKKPGSSVKVSCKASGYTFTSYHIQWV |
| Chain VH1-69 | | RQAPGQGLEWMGWIYPGDGSTQYNEKFKGRTTLTAD |
| (H27Y, H30S, | | KSTSTAYMELSSLRSEDTAVYYCAREGTYYAMDYWG |
| VH67T, H69L)) | | QGTLVTVSS |
| Hz.BB7.2VH1- | 69 | DVVMTQTPLSLSVTPGQPASISCRSSQSIVHSNGNTYLE |
| 3_A18 variable | | WYLQKPGQSPQLLIYKVSNRFSGVPDRFSGSGSGTDFT |
| light chain | | LKISRVEAEDVGVYYCFQGSHVPRTFGGGTKVEIK |
| Hz.BB7.2 Heavy | 70 | QVQLVQSGAEVKKPGASVKVSCKASGYTFTSYHIQWV |
| Chain VH1-3) | | RQAPGQRLEWMGWIYPGDGSTQYNEKFKGRVTITRD |
| | | TSASTAYMELSSLRSEDTAVYYCAREGTYYAMDYWG |
| | | QGTLVTVSS |
| Hz.BB7.2VH1-3 | 71 | DVVMTQTPLSLSVTPGQPASISCRSSQSIVHSNGNTYLE |
| (48)_A18 | | WYLQKPGQSPQLLIYKVSNRFSGVPDRFSGSGSGTDFT |
| variable light | | LKISRVEAEDVGVYYCFQGSHVPRTFGGGTKVEIK |
| chain | | |
| Hz.BB7.2 Heavy | 72 | QVQLVQSGAEVKKPGASVKVSCKASGYTFTSYHIQWV |
| Chain VH1-3 | | RQAPGQRLEWIGWIYPGDGSTQYNEKFKGRVTITRDT |
| (H48I)) | | SASTAYMELSSLRSEDTAVYYCAREGTYYAMDYWGQ |
| | | GTLVTVSS |
| Hz.BB7.2VH1-3 | 73 | DVVMTQTPLSLSVTPGQPASISCRSSQSIVHSNGNTYLE |
| (67)_A18 | | WYLQKPGQSPQLLIYKVSNRFSGVPDRFSGSGSGTDFT |
| variable light | | LKISRVEAEDVGVYYCFQGSHVPRTFGGGTKVEIK |
| chain | | |
| (Hz.BB7.2 Light | | |
| chain VKA18) | | |
| Hz.BB7.2 Heavy | 74 | QVQLVQSGAEVKKPGASVKVSCKASGYTFTSYHIQWV |
| Chain VH1-3 | | RQAPGQRLEWMGWIYPGDGSTQYNEKFKGRTTITRD |
| (H67T)) | | TSASTAYMELSSLRSEDTAVYYCAREGTYYAMDYWG |
| | | QGTLVTVSS |
| Hz.BB.2VH1-3 | 75 | DVVMTQTPLSLSVTPGQPASISCRSSQSIVHSNGNTYLE |
| (69)_A18 | | WYLQKPGQSPQLLIYKVSNRFSGVPDRFSGSGSGTDFT |
| variable light | | LKISRVEAEDVGVYYCFQGSHVPRTFGGGTKVEIK |
| chain | | |
| Hz.BB7.2 Heavy | 76 | QVQLVQSGAEVKKPGASVKVSCKASGYTFTSYHIQWV |
| Chain VH1-3 | | RQAPGQRLEWMGWIYPGDGSTQYNEKFKGRVTLTRD |
| (H69L)) | | TSASTAYMELSSLRSEDTAVYYCAREGTYYAMDYWG |
| | | QGTLVTVSS |
| Hz.BB7.2VH1-3 | 77 | DVVMTQTPLSLSVTPGQPASISCRSSQSIVHSNGNTYLE |
| (71)_A18 | | WYLQKPGQSPQLLIYKVSNRFSGVPDRFSGSGSGTDFT |
| variable light | | LKISRVEAEDVGVYYCFQGSHVPRTFGGGTKVEIK |
| chain | | |
| Hz.BB7.2 VH1-3 | 78 | QVQLVQSGAEVKKPGASVKVSCKASGYTFTSYHIQWV |
| (71)_variable | | RQAPGQRLEWMGWIYPGDGSTQYNEKFKGRVTITAD |
| heavy chain | | TSASTAYMELSSLRSEDTAVYYCAREGTYYAMDYWG |
| | | QGTLVTVSS |
| Hz.BB7.2VH1-3 | 79 | DVVMTQTPLSLSVTPGQPASISCRSSQSIVHSNGNTYLE |
| (73)_A18 | | WYLQKPGQSPQLLIYKVSNRFSGVPDRFSGSGSGTDFT |
| variable light | | LKISRVEAEDVGVYYCFQGSHVPRTFGGGTKVEIK |
| chain | | |
| Hz.BB7.2VH1-3 | 80 | QVQLVQSGAEVKKPGASVKVSCKASGYTFTSYHIQWV |
| (73)_A18 | | RQAPGQRLEWMGWIYPGDGSTQYNEKFKGRVTITRD |
| variable heavy | | KSASTAYMELSSLRSEDTAVYYCAREGTYYAMDYWG |
| chain | | QGTLVTVSS |
| MWB 1.2 variable | 163 | QSALTQPPSASGSPGQSVTISCTGTSSDVGGYKYVSWY |
| light chain | | QQHPGKAPKLMIYEVNKRPSGVPDRFSGSKSGNTASLT |
| | | VSGLQAEDEADYYCSSYAGSNNWVFGGGTKLTVL |
| MWB 1.2 variable | 164 | QVQLVESGGGVVQPGGSLRLSCAASGFTFSTYGMHWV |
| heavy chain | | RQAPGKGLEWVASISYDGSNKYYADSGQGRFTISRDTS |
| | | KNSLYLQMNSLRAEDTAVYYCAIGIYGAYSFDYWGQG |
| | | TLVTVSS |
| SN66E3.2 | 165 | DIVMTQSPDSLAVSLGERATISCKSSQSVLYSSNNKNYL |
| variable light | | AWYQQKPGQPPKLLIYWASTRESGVPDRFSGSGSGTDF |
| chain | | TLTISSLQAEDVAVYYCQQYYGTPFTFGGGTKVEIK |
| SN66E3.2 | 166 | QVQLVQSGAEVKKPGASVKVSCKASGYTFTDYYLHW |
| variable heavy | | VRQAPGQGLEWMGWINPYTGGTNYAQKFQGRVTMT |
| chain | | RDTSISTAYMELSGLTSDDTAVYYCARAGASYYDFWS |
| | | GWVFDYWGQGTLVTVSS |
| MWB1.1 | 273 | QVQLVESGGGVVQPGGSLRLSCAASGFTFSTYGMHWV |
| scFvVH_VL | | RQAPGKGLEWVASISYDGSNKYYADSGQGRFTISRDTS |
| | | KNSLYLQMNSLRAEDTAVYYCAIGIYGAYSFDYWGQG |
| | | TLVTVSSGGGGSGGGGSGGGGSQSALTQPPSASGSPGQ |
| | | SVTISCTGTSSDVGGYKYVSWYQHHPDKAPKLMIYEV |
| | | NKRPSGVPDRFSGSKSDNTASLTVSGLQAEDEADYYCS |
| | | SYAGSNNWVFGGGTKLTVL |
| MWB1.2scFvVH_ | 274 | QVQLVESGGGVVQPGGSLRLSCAASGFTFSTYGMHWV |
| VL | | RQAPGKGLEWVASISYDGSNKYYADSGQGRFTISRDTS |
| | | KNSLYLQMNSLRAEDTAVYYCAIGIYGAYSFDYWGQG |
| | | TLVTVSSGGGGSGGGGSGGGGSQSALTQPPSASGSPGQ |
| | | SVTISCTGTSSDVGGYKYVSWYQQHPGKAPKLMIYEV |
| | | NKRPSGVPDRFSGSKSGNTASLTVSGLQAEDEADYYCS |
| | | SYAGSNNWVFGGGTKLTVL |
| SN66E3.3 | 283 | DIVMTQSPDSLAVSLGERATISCKSSQSVLYSSNNKNYL |
| Variable Light | | AWYQQKPGQPPKLLIYWASTRESGVPDRFSGSGSGTDF |
| chain | | TLTISSLQAEDVAVYYCQQYYGTPFTFGGGTKVEIK |
| SN66E3.3 | 284 | QVQLVQSGAEVKKPGASVKVSCKASGYTFTDYYLHW |
| variable Heavy | | VRQAPGQGLEWMGWINPYTGGTNYAQKFQGRVTMT |
| chain | | RDTSISTAYMELSRLRSEDTAVYYCARAGASYYDFWS |
| | | GWVFDYWGQGTLVTVSS |

In some embodiments, the orientation of the iCAR VH and VL regions is VH-VL. In some embodiments, the orientation of the iCAR VH and VL regions is VL-VH.

In some embodiments, the iCAR scFv comprises a linker that covalently connects the VH and the VL to form the iCAR scFv.

In some embodiments, the heavy and light chains of the scFv are covalently connected via a linker. In some embodiments, the linker is a gly-ser polypeptide linker, i.e., a peptide that consists of glycine and serine residues. Exemplary gly-ser polypeptide linkers comprise the amino acid sequence Ser(Gly₄Ser)ₙ, as well as (Gly₄Ser)ₙ and/or (Gly₄Ser₃)ₙ. In some embodiments, n=1. In some embodiments, n=2. In some embodiments, n=3, i.e., Ser(Gly₄Ser)₃. In some embodiments, n=4, i.e., Ser(Gly₄Ser)₄. In some embodiments, n=5. In some embodiments, n=6. In some embodiments, n=7. In some embodiments, n=8. In some embodiments, n=9. In some embodiments, n=10. Another exemplary gly-ser polypeptide linker comprises the amino acid sequence Ser(Gly₄Ser)ₙ. In some embodiments, n=1. In some embodiments, n=2. In some embodiments, n=3. In another embodiment, n=4. In some embodiments, n=5. In some embodiments, n=6. Another exemplary gly-ser polypeptide linker comprises (Gly₄Ser)ₙ. In some embodiments, n=1. In some embodiments, n=2. In some embodiments, n=3. In some embodiments, n=4. In some embodiments, n=5. In some embodiments, n=6. Another exemplary gly-ser polypeptide linker comprises (Gly₃Ser)ₙ. In some embodiments, n=1. In some embodiments, n=2. In some embodiments, n=3. In some embodiments, n=4. In another embodiment, n=5. In yet another embodiment, n=6. Another exemplary gly-ser polypeptide linker comprises (Gly₄Ser₃)ₙ. In some embodiments, n=1. In some embodiments, n=2. In some embodiments, n=3. In some embodiments, n=4. In some embodiments, n=5. In some embodiments, n=6. Another exemplary gly-ser polypeptide linker comprises (Gly₃Ser)ₙ. In some embodiments, n=1. In some embodiments, n=2. In some embodiments, n=3. In some embodiments, n=4. In another embodiment, n=5. In yet another embodiment, n=6.

In some embodiments, the iCAR comprises a GS based linker sequence, connecting the VH and VL or the VL and VH to form the scFv. In some embodiments, the GS linker comprises GGGGS (SEQ ID NO:153). In some embodiments, the iCAR comprises a Whitlow linker sequence, e.g., GSTSGSGKPGSGEGSTKG (SEQ ID NO:82). In some embodiments, the iCAR comprises the Vh and Vl sequences in the Vh-Vl orientation. In some embodiments, the iCAR comprises the Vh and Vl sequences in the Vl-Vh orientation. In some embodiments, the iCAR comprises a linker between the Vh and Vl sequences. In some embodiments, the iCAR does not comprise a linker between the Vh and Vl sequences.

TABLE 3
iCAR linkers

| Sequence | SEQ ID | |
|---|---|---|
| Information | NO | Amino acid sequence |
| (G4S)X3 linker | 81 | GGGGSGGGGSGGGG |
| | | S |
| Whitlow linker | 82 | GSTSGSGKPGSGEGST |
| | | KG |
| PD1 linker | 83 | DFQWREKTPEPPVPC |
| | | VPEQ |
| G4S | 153 | GGGGS |

In some embodiments, the iCAR scFv comprises a linker. In some embodiments, the iCAR scFv is selected from the group consisting of BB7.2 scFv (SEQ ID NO: 167), 3PF12 scFv (SEQ ID NO: 168), SN66E3.1 scFv (SEQ ID NO: 169), SN66E3.2 scFv (SEQ ID NO: 285), SN66E3.3 scFv (SEQ ID NO: 286), Hz BB7.2.1 scFv (SEQ ID NO: 287), and Hz BB7.2.2 scFv (SEQ ID NO: 288). In some embodiments, the iCAR scFv is BB7.2 scFv (SEQ ID NO: 167). In some embodiments, the iCAR scFv is 3PF12 scFv (SEQ ID NO: 168). In some embodiments, the iCAR scFv is SN66E3.1 scFv (SEQ ID NO: 169). In some embodiments, the iCAR scFv is SN66E3.2 scFv (SEQ ID NO: 285). In some embodiments, the iCAR scFv is SN66E3.3 scFv (SEQ ID NO: 286). In some embodiments, the iCAR scFv is Hz BB7.2.1 scFv (SEQ ID NO: 287). In some embodiments, the iCAR scFv is Hz BB7.2.2 scFv (SEQ ID NO: 288).

TABLE 4
iCAR scFv sequences with linkers

| Sequence | SEQ ID | |
|---|---|---|
| Information | NO | Amino acid sequence |
| BB7.2 scFv | 167 | QVQLQQSGPELVKPGASVKMSCKASGYTFTSYHIQWVK |
| | | QRPGQGLEWIGWIYPGDGSTQYNEKFKGKTTLTADKSSS |
| | | TAYMLLSSLTSEDSAIYFCAREGTYYAMDYWGQGTSVT |
| | | VSSGGGGSGGGGSGGGGSDVLMTQTPLSLPVSLGDQVSI |
| | | SCRSSQSIVHSNGNTYLEWYLQKPGQSPKLLIYKVSNRFS |
| | | GVPDRFSGSGSGTDFTLKISRVEAEDLGVYYCFQGSHVP |
| | | RTFGGGTKLEIK |
| 3PF12 scFv | 168 | QVQLVQSGGGVVQPGGSLRVSCAASGVTLSDYGMHWV |
| | | RQAPGKGLEWMAFIRNDGSDKYYADSVKGRFTISRDNS |
| | | KKTVSLQMSSLRAEDTAVYYCAKNGESGPLDYWYFDL |
| | | WGRGTLVTVSSGGGGSGGGGSGGGGSDIVMTQSPSFLS |
| | | ASVGDRVTITCRASHGINNYLAWYQQKPGKAPKLLIYA |
| | | ASTLQSGVPSRESGSGSGTEFTLTISSLQPEDFATYYCQQ |
| | | YDSYPPTFGRGTKVEIK |
| SN66E3.1 scFv | 169 | QVQLVQSGAEVKKPGASVKVSCKASGYTFTDYYLHWV |
| | | RQAPGQGLEWMGWINPYTGGTNYAQKFQGRVTMTRD |
| | | ASISTVYMELSGLTSDDTAVHFCARAGASYYDFWSGWV |
| | | FDYWGQGTLVTVSSGGGGSGGGGSGGGGSDIVMTQSPD |
| | | SLAVSLGERATISCKSSQSVLYSSNNKNYLAWYQQKLG |
| | | QPPKLLIYWASTRESGVPDRFSGSGSGTNFTLTISSLQAE |
| | | NVAVYYCQQYYGTPFTFGGGTKVEIK |
| SN66E3.2 scFv | 285 | DIVMTQSPDSLAVSLGERATISCKSSQSVLYSSNNKNYL |
| | | AWYQQKPGQPPKLLIYWASTRESGVPDRFSGSGSGTDFT |
| | | LTISSLQAEDVAVYYCQQYYGTPFTFGGGTKVEIKGGGG |
| | | SGGGGSGGGGSQVQLVQSGAEVKKPGASVKVSCKASG |
| | | YTFTDYYLHWVRQAPGQGLEWMGWINPYTGGTNYAQ |
| | | KFQGRVTMTRDTSISTAYMELSGLTSDDTAVYYCARAG |
| | | ASYYDFWSGWVFDYWGQGTLVTVSS |
| SN66E3.3 scFv | 286 | DIVMTQSPDSLAVSLGERATISCKSSQSVLYSSNNKNYL |
| | | AWYQQKPGQPPKLLIYWASTRESGVPDRFSGSGSGTDFT |
| | | LTISSLQAEDVAVYYCQQYYGTPFTFGGGTKVEIKGGGG |
| | | SGGGGSGGGGSQVQLVQSGAEVKKPGASVKVSCKASG |
| | | YTFTDYYLHWVRQAPGQGLEWMGWINPYTGGTNYAQ |
| | | KFQGRVTMTRDTSISTAYMELSRLRSEDTAVYYCARAG |
| | | ASYYDFWSGWVFDYWGQGTLVTVSS |
| Hz BB7.2.1 scFv | 287 | QVQLVQSGAEVKKPGSSVKVSCKASGYTFTSYHIQWVR |
| | | QAPGQGLEWMGWIYPGDGSTQYNEKFKGRTTITADKST |
| | | STAYMELSSLRSEDTAVYYCAREGTYYAMDYWGQGTL |
| | | VTVSSGGGGSGGGGSGGGGSDVVMTQTPLSLSVTPGQP |
| | | ASISCRSSQSIVHSNGNTYLEWYLQKPGQSPQLLIYKVSN |
| | | RFSGVPDRFSGSGSGTDFTLKISRVEAEDVGVYYCFQGS |
| | | HVPRTFGGGTKVEIK |
| HzBB7.2.2 scFV | 288 | QVQLVQSGAEVKKPGASVKVSCKASGYTFTSYHIQWVR |
| | | QAPGQRLEWIGWIYPGDGSTQYNEKFKGRVTITRDTSAS |
| | | TAYMELSSLRSEDTAVYYCAREGTYYAMDYWGQGTLV |
| | | TVSSGGGGSGGGGSGGGGSDVVMTQTPLSLSVTPGQPA |
| | | SISCRSSQSIVHSNGNTYLEWYLQKPGQSPQLLIYKVSNR |
| | | FSGVPDRFSGSGSGTDFTLKISRVEAEDVGVYYCFQGSH |
| | | VPRTFGGGTKVEIK |

In some embodiments, the iCAR scFv linker is a gly-ser polypeptide linker, i.e., a peptide that consists of glycine and serine residues. Exemplary gly-ser polypeptide linkers comprise the amino acid sequence Ser(Gly₄Ser)ₙ, as well as (Gly₄Ser)ₙ and/or (Gly₄Ser₃)ₙ. In some embodiments, n=1. In some embodiments, n=2. In some embodiments, n=3, i.e., Ser(Gly₄Ser)₃. In some embodiments, n=4, i.e., Ser(Gly₄Ser)₄. In some embodiments, n=5. In some embodiments, n=6. In some embodiments, n=7. In some embodiments, n=8. In some embodiments, n=9. In some embodiments, n=10. Another exemplary gly-ser polypeptide linker comprises the amino acid sequence Ser(Gly₄Ser)ₙ. In some embodiments, n=1. In some embodiments, n=2. In some embodiments, n=3. In another embodiment, n=4. In some embodiments, n=5. In some embodiments, n=6. Another exemplary gly-ser polypeptide linker comprises (Gly₄Ser)ₙ. In some embodiments, n=1. In some embodiments, n=2. In some embodiments, n=3. In some embodiments, n=4. In some embodiments, n=5. In some embodiments, n=6. Another exemplary gly-ser polypeptide linker comprises (Gly₃Ser)ₙ. In some embodiments, n=1. In some embodiments, n=2. In some embodiments, n=3. In some embodiments, n=4. In another embodiment, n=5. In yet another embodiment, n=6. Another exemplary gly-ser polypeptide linker comprises (Gly₄Ser₃)ₙ. In some embodiments, n=1. In some embodiments, n=2. In some embodiments, n=3. In some embodiments, n=4. In some embodiments, n=5. In some embodiments, n=6. Another exemplary gly-ser polypeptide linker comprises (Gly₃Ser)ₙ. In some embodiments, n=1. In some embodiments, n=2. In some embodiments, n=3. In some embodiments, n=4. In another embodiment, n=5. In yet another embodiment, n=6.

2. iCAR Portion: Hinge Domain

In some embodiments, the bicistronic construct comprises an iCAR portion comprising a hinge domain component. In some embodiments, the hinge domain comprises a hinge selected from the group consisting of a PD-1 hinge domain, a CD28 hinge domain, and a CD8 hinge domain (including a CD8a hinge domain) a LIR1 Ig3-4 hinge domain, a LIR1 Ig-4 hinge domain, a LIR1 52 aa hinge domain, a LIR1 36 aa hinge domain, a LIR1 30 aa hinge domain, a LIR1 8 aa hinge domain, a CD33 hinge domain, and a KIR2DL1 hinge domain. In some embodiments, the hinge domain is a PD-1 hinge (SEQ ID NO: 86). In some embodiments, the hinge domain is a CD28 hinge domain (SEQ ID NO:85). In some embodiments, the vector comprises a CD8 hinge domain. In some embodiments, the vector comprises a CD8a hinge domain (SEQ ID NO:84). In some embodiments, the vector comprises a LIR1 Ig3-4 hinge domain (SEQ ID NO:87). In some embodiments, the vector comprises a LIR Ig-4 hinge domain (SEQ ID NO:88). In some embodiments, the vector comprises a LIR1 52 aa hinge domain (SEQ ID NO:89). In some embodiments, the vector comprises a LIR1 36 aa hinge domain (SEQ ID NO:90). In some embodiments, the vector comprises a LIR1 30 aa hinge domain (SEQ ID NO:91). In some embodiments, the vector comprises a LIR1 8 aa hinge domain (SEQ ID NO:92). In some embodiments, the vector comprises a CD33 hinge domain (SEQ ID NO:93). In some embodiments, the vector comprises a KIR2DL1 hinge domain (SEQ ID NO:94). In some embodiments, the iCAR comprises a LIR1 26 aa (SEQ ID NO: 289). In some embodiments, the iCAR comprises PD-1 (47) (SEQ ID NO: 290). In some embodiments, the iCAR comprises PD-1 (42) (SEQ ID NO: 291). In some embodiments, the iCAR comprises PD-1(36) (SEQ ID NO: 292). In some embodiments, the iCAR comprises PD-1 (30) (SEQ ID NO: 293). In some embodiments, the iCAR comprises PD-1 (26) (SEQ ID NO: 294). In some embodiments, the iCAR comprises PD-1 (20) (SEQ ID NO: 295).

TABLE 5
iCAR hinge sequences

| Sequence | SEQ ID | |
|---|---|---|
| Information | NO | Amino acid sequence |
| CD8 alpha | 84 | TTTPAPRPPTPAPTIASQPLSLRPEACRPAAGGAVHTR |
| | | GLDFACD |
| CD28 | 85 | IEVMYPPPYLDNEKSNGTIIHVKGKHLCPSPLFPGPSK |
| | | P |
| PD-1 | 86 | TERRAEVPTAHPSPSPRPAGQFQTLV |
| LIR1 Ig3-4 | 87 | VSKKPSLSVQPGPIVAPEETLTLQCGSDAGYNRFVLY |
| | | KDGERDFLQLAGAQPQAGLSQANFTLGPVSRSYGGQ |
| | | YRCYGAHNLSSEWSAPSDPLDILIAGQFYDRVSLSVQ |
| | | PGPTVASGENVTLLCQSQGWMQTFLLTKEGAADDP |
| | | WRLRSTYQSQKYQAEFPMGPVTSAHAGTYRCYGSQ |
| | | SSKPYLLTHPSDPLELVVSGPSGGPSSPTTGPTSTSGPE |
| | | DQPLTPTGSDPQSGLGRHLGV |
| LIR1 Ig-4 | 88 | PLDILIAGQFYDRVSLSVQPGPTVASGENVTLLCQSQ |
| | | GWMQTFLLTKEGAADDPWRLRSTYQSQKYQAEFPM |
| | | GPVTSAHAGTYRCYGSQSSKPYLLTHPSDPLELVVSG |
| | | PSGGPSSPTTGPTSTSGPEDQPLTPTGSDPQSGLGRHL |
| | | GV |
| LIR1 52 aa | 89 | HPSDPLELVVSGPSGGPSSPTTGPTSTSGPEDQPLTPT |
| | | GSDPQSGLGRHLGV |
| LIR1 36 aa | 90 | PSSPTTGPTSTSGPEDQPLTPTGSDPQSGLGRHLGV |
| LIR1 30 aa | 91 | GPTSTSGPEDQPLTPTGSDPQSGLGRHLGV |
| LIR1 8 aa | 92 | GLGRHLGV |
| CD33 | 93 | LNVTYVPQNPTTGIFPGDGSGKQETRAGVVH |
| KIR2DL1 | 94 | PYEWSKSSDPLLVSVTGNPSNSWPSPTEPSSKTGNPR |
| | | HLH |
| LIR1 26 aa | 289 | TSGPEDQPLTPTGSDPQSGLGRHLGV |
| PD-1 (47) | 290 | GAISLAPKAQIKESLRAELRVTERRAEVPTAHPSPSPR |
| | | PAGQFQTLV |
| PD-1 (42) | 291 | APKAQIKESLRAELRVTERRAEVPTAHPSPSPRPAGQ |
| | | FQTLV |
| PD-1 (36) | 292 | KESLRAELRVTERRAEVPTAHPSPSPRPAGQFQTLV |
| PD-1 (30) | 293 | ELRVTERRAEVPTAHPSPSPRPAGQFQTLV |
| PD-1 (26) | 294 | TERRAEVPTAHPSPSPRPAGQFQTLV |
| PD-1 (20) | 295 | VPTAHPSPSPRPAGQFQTLV |

3. iCAR Portion: Transmembrane Domain

In some embodiments, the bicistronic construct comprises an iCAR portion comprising a transmembrane (TM) domain component. In some embodiments, the TM domain comprises a TM domain selected from the group consisting of a PD-1 TM domain, a CD28 TM domain, a CD8 TM domain (including a CD8a TM domain), a LIR1 TM domain, a CD33 TM domain, and a KIR2DL1 TM domain. In some embodiments, the TM domain is a PD-1 TM domain (SEQ ID NO:97). In some embodiments, the TM domain is a CD28 TM domain (SEQ ID NO:96). In some embodiments, the vector comprises a CD8 TM domain. In some embodiments, the vector comprises a CD8a TM domain (SEQ ID NO:95). In some embodiments, the vector comprises a LIR1 TM domain (SEQ ID NO:98). In some embodiments, the vector comprises a CD33 TM domain (SEQ ID NO:99). In some embodiments, the vector comprises a KIR2DL1 TM domain (SEQ ID NO:100).

TABLE 6
iCAR transmembrane sequences

| Sequence | SEQ ID | |
|---|---|---|
| Information | NO | Amino acid sequence |
| CD8 alpha | 95 | IYIWAPLAGTCGVLLLSLVITLYC |
| CD28 | 96 | FWVLVVVGGVLACYSLLVTVAFIIFWV |
| PD-1 | 97 | VGVVGGLLGSLVLLVWVLAVI |
| LIR1 | 98 | VIGILVAVILLLLLLLLLFLI |
| CD33 | 99 | GAIGGAGVTALLALCLCLIFFIV |
| KIR2DL1 | 100 | ILIGTSVVIILFILLFFLL |

4. iCAR Portion: Inhibitory Domain

In some embodiments, the bicistronic construct comprises an iCAR portion comprising an inhibitory domain component. In some embodiments, the iCAR portion comprises an inhibitory domain. In some embodiments, the inhibitory domain is selected from the group consisting of PD-1, KIR2DL1, KIR2DL2, KIR2DL3, KIR2DL4, KIR2DL5A, KIR3DL1, KIR3DL2, KIR3DL3, LAIR1, CD22, CD33, SIGLEC5, SIGLEC6, SIGLEC7, SIGLEC8, SIGLEC9, SIGLEC10, SIGLEC1I, SIGLEC12, PECAM1/CD31, CD200R1, FCRL1, FCRL2, FCRL3, FCRL4, FCRL5, SLAMF1, SLAMF5, BTLA, LAG3, 2B4, CD160, CEACAM1, TIM3, VISTA, TIGIT, SIRPalpha, FcγRIIB, CD5, CD300a, CD300f, LIR1, LIR2, LIR3, LIR5, LIR8, Ly9, 2×PD1(G4S), 2×PD1(PD1), PVRIg, and AA2ARKIR2DL1, synthetic LIR1, and synthetic PD-1. In some embodiments, the inhibitory domain is KIR2DL1 (SEQ ID NO:102). In some embodiments, the inhibitory domain is LIR1 (SEQ ID NO:143). In some embodiments, the inhibitory domain is PD-1 (SEQ ID NO:101). In some embodiments, the inhibitory domain is KIR2DL2 (SEQ ID NO:103). In some embodiments, the inhibitory domain is KIR2DL3 (SEQ ID NO:104). In some embodiments, the inhibitory domain is KIR2DL4 (SEQ ID NO:105). In some embodiments, the inhibitory domain is KIR2DL5A (SEQ ID NO:106). In some embodiments, the inhibitory domain is KIR3DL1 (SEQ ID NO:107). In some embodiments, the inhibitory domain is KIR3DL2 (SEQ ID NO:108). In some embodiments, the inhibitory domain is KIR3DL3 (SEQ ID NO:109). In some embodiments, the inhibitory domain is LAIR1 (SEQ ID NO:110). In some embodiments, the inhibitory domain is CD22 (SEQ ID NO:111). In some embodiments, the inhibitory domain is CD33 (SEQ ID NO:112). In some embodiments, the inhibitory domain is SIGLEC5 (SEQ ID NO:113). In some embodiments, the inhibitory domain is SIGLEC6 (SEQ ID NO:114). In some embodiments, the inhibitory domain is SIGLEC7 (SEQ ID NO:115). In some embodiments, the inhibitory domain is SIGLEC8 (SEQ ID NO:116). In some embodiments, the inhibitory domain is SIGLEC9 (SEQ ID NO:117). In some embodiments, the inhibitory domain is SIGLEC10 (SEQ ID NO:118). In some embodiments, the inhibitory domain is SIGLEC1I (SEQ ID NO:119). In some embodiments, the inhibitory domain is SIGLEC12 (SEQ ID NO:120). In some embodiments, the inhibitory domain is PECAM1/CD31 (SEQ ID NO:121). In some embodiments, the inhibitory domain is CD200R1 (SEQ ID NO:122). In some embodiments, the inhibitory domain is FCRL1 (SEQ ID NO:123). In some embodiments, the inhibitory domain is FCRL2 (SEQ ID NO:124). In some embodiments, the inhibitory domain is FCRL3 (SEQ ID NO:125). In some embodiments, the inhibitory domain is FCRL4 (SEQ ID NO:126). In some embodiments, the inhibitory domain is FCRL5 (SEQ ID NO:127). In some embodiments, the inhibitory domain is SLAMF1 (SEQ ID NO:128). In some embodiments, the inhibitory domain is SLAMF5 (SEQ ID NO:129). In some embodiments, the inhibitory domain is BTLA (SEQ ID NO:130). In some embodiments, the inhibitory domain is LAG3 (SEQ ID NO:131). In some embodiments, the inhibitory domain is 2B4 (SEQ ID NO:132). In some embodiments, the inhibitory domain is CD160 (SEQ ID NO:133). In some embodiments, the inhibitory domain is CEACAM1 (SEQ ID NO:134). In some embodiments, the inhibitory domain is TIM3 (SEQ ID NO:135). In some embodiments, the inhibitory domain is VISTA (SEQ ID NO:136). In some embodiments, the inhibitory domain is TIGIT (SEQ ID NO:137). In some embodiments, the inhibitory domain is SIRPalpha (SEQ ID NO:138). In some embodiments, the inhibitory domain is FcγRIIB (SEQ ID NO:139). In some embodiments, the inhibitory domain is CD5 (SEQ ID NO:140). In some embodiments, the inhibitory domain is CD300a (SEQ ID NO:141). In some embodiments, the inhibitory domain is CD300f (SEQ ID NO:142). In some embodiments, the inhibitory domain is LIR1 (SEQ ID NO:143). In some embodiments, the inhibitory domain is LIR2 (SEQ ID NO:144). In some embodiments, the inhibitory domain is LIR3 (SEQ ID NO:145). In some embodiments, the inhibitory domain is LIR5 (SEQ ID NO:146). In some embodiments, the inhibitory domain is LIR8 (SEQ ID NO:147). In some embodiments, the inhibitory domain is Ly9 (SEQ ID NO:148). In some embodiments, the inhibitory domain is 2×PD1(G4S) (SEQ ID NO:149). In some embodiments, the inhibitory domain is 2×PD1(PD1) (SEQ ID NO:150). In some embodiments, the inhibitory domain is PVRIg (SEQ ID NO:151). In some embodiments, the inhibitory domain is AA2AR (SEQ ID NO:152).

TABLE 7
iCAR inhibitory domain sequences

| Sequence | SEQ ID | |
|---|---|---|
| Information | NO | Amino acid sequence |
| PD-1 | 101 | CSRAARGTIGARRTGQPLKEDPSAVPVFSVDYGELDFQW |
| | | REKTPEPPVPCVPEQTEYATIVFPSGMGTSSPARRGSADGP |
| | | RSAQPLRPEDGHCSWPL |
| KIR2DL1 | 102 | HRWCSNKKNAAVMDQESAGNRTANSEDSDEQDPQEVTY |
| | | TQLNHCVFTQRKITRPSQRPKTPPTDIIVYTELPNAESRSKV |
| | | VSCP |
| KIR2DL2 | 103 | HRWCSNKKNAAVMDQESAGNRTANSEDSDEQDPQEVTY |
| | | TQLNHCVFTQRKITRPSQRPKTPPTDIIVYAELPNAESRSK |
| | | VVSCP |
| KIR2DL3 | 104 | HRWCCNKKNAVVMDQEPAGNRTVNREDSDEQDPQEVT |
| | | YAQLNHCVFTQRKITRPSQRPKTPPTDIIVYTELPNAEP |
| KIR2DL4 | 105 | RWCSKKKDAAVMNQEPAGHRTVNREDSDEQDPQEVTYA |
| | | QLDHCIFTQRKITGPSQRSKRPSTDTSVCIELPNAEPRALSP |
| | | AHEHHSQALMGSSRETTALSQTQLASSNVPAAGI |
| KIR2DL5A | 106 | LHCCCSNKKNAAVMDQEPAGDRTVNREDSDDQDPQEVT |
| | | YAQLDHCVFTQTKITSPSQRPKTPPTDTTMYMELPNAKPR |
| | | SLSPAHKHHSQALRGSSRETTALSQNRVASSHVPAAGI |
| KIR3DL1 | 107 | HLWCSNKKNAAVMDQEPAGNRTANSEDSDEQDPEEVTY |
| | | AQLDHCVFTQRKITRPSQRPKTPPTDTILYTELPNAKPRSK |
| | | VVSCP |
| KIR3DL2 | 108 | YRWCSNKKNAAVMDQEPAGDRTVNRQDSDEQDPQEVT |
| | | YAQLDHCVFIQRKISRPSQRPKTPLTDTSVYTELPNAEPRS |
| | | KVVSCPRAPQSGLEGVF |
| KIR3DL3 | 109 | HRWCANKKNAVVMDQEPAGNRTVNREDSDEQDPQEVT |
| | | YAQLNHCVFTQRKITRPSQRPKTPPTDTSV |
| LAIR1 | 110 | HRQNQIKQGPPRSKDEEQKPQQRPDLAVDVLERTADKAT |
| | | VNGLPEKDRETDTSALAAGSSQEVTYAQLDHWALTQRTA |
| | | RAVSPQSTKPMAESITYAAVARH |
| CD22 | 111 | KLQRRWKRTQSQQGLQENSSGQSFFVRNKKVRRAPLSEG |
| | | PHSLGCYNPMMEDGISYTTLRFPEMNIPRTGDAESSEMQR |
| | | PPPDCDDTVTYSALHKRQVGDYENVIPDFPEDEGIHYSELI |
| | | QFGVGERPQAQENVDYVILKH |
| CD33 | 112 | KTHRRKAARTAVGRNDTHPTTGSASPKHQKKSKLHGPTE |
| | | TSSCSGAAPTVEMDEELHYASLNFHGMNPSKDTSTEYSEV |
| | | RTQ |
| SIGLEC5 | 113 | KARRKQAAGRPEKMDDEDPIMGTITSGSRKKPWPDSPGD |
| | | QASPPGDAPPLEEQKELHYASLSFSEMKSREPKDQEAPSTT |
| | | EYSEIKTSK |
| SIGLEC6 | 114 | RVKTRRKKAAQPVQNTDDVNPVMVSGSRGHQHQFQTGI |
| | | VSDHPAEAGPISEDEQELHYAVLHFHKVQPQEPKVTDTEY |
| | | SEIKIHK |
| SIGLEC7 | 115 | RSCRKKSARPAADVGDIGMKDANTIRGSASQGNLTESWA |
| | | DDNPRHHGLAAHSSGEEREIQYAPLSFHKGEPQDLSGQEA |
| | | TNNEYSEIKIPK |
| SIGLEC8 | 116 | RSCRKKSARPAAGVGDTGMEDAKAIRGSASQGPLTESWK |
| | | DGNPLKKPPPAVAPSSGEEGELHYATLSFHKVKPQDPQGQ |
| | | EATDSEYSEIKIHKRETAETQACLRNHNPSSKEVRG |
| SIGLEC9 | 117 | VRSCRKKSARPAAGVGDTGIEDANAVRGSASQGPLTEPW |
| | | AEDSPPDQPPPASARSSVGEGELQYASLSFQMVKPWDSRG |
| | | QEATDTEYSEIKIHR |
| SIGLEC10 | 118 | KILPKRRTQTETPRPRFSRHSTILDYINVVPTAGPLAQKRN |
| | | QKATPNSPRTPLPPGAPSPESKKNQKKQYQLPSFPEPKSST |
| | | QAPESQESQEELHYATLNFPGVRPRPEARMPKGTQADYA |
| | | EVKFQ |
| SIGLEC11 | 119 | KICRKEARKRAAAEQDVPSTLGPISQGHQHECSAGSSQDH |
| | | PPPGAATYTPGKGEEQELHYASLSFQGLRLWEPADQEAPS |
| | | TTEYSEIKIHTGQPLRGPGFGLQLEREMSGMVPK |
| SIGLEC12 | 120 | RSCRKKSARPAVGVGDTGMEDANAVRGSASQGPLIESPA |
| | | DDSPPHHAPPALATPSPEEGEIQYASLSFHKARPQYPQEQE |
| | | AIGYEYSEINIPK |
| PECAM1/CD31 | 121 | KCYFLRKAKAKQMPVEMSRPAVPLLNSNNEKMSDPNME |
| | | ANSHYGHNDDVRNHAMKPINDNKEPLNSDVQYTEVQVS |
| | | SAESHKDLGKKDTETVYSEVRKAVPDAVESRYSRTEGSL |
| | | DGT |
| CD200R1 | 122 | KVNGCRKYKLNKTESTPVVEEDEMQPYASYTEKNNPLYD |
| | | TTNKVKASEALQSEVDTDLHTL |
| FCRL1 | 123 | GLKRKIGRRSARDPLRSLPSPLPQEFTYLNSPTPGQLQPIYE |
| | | NVNVVSGDEVYSLAYYNQPEQESVAAETLGTHMEDKVS |
| | | LDIYSRLRKANITDVDYEDAM |
| FCRL2 | 124 | HKISGESSATNEPRGASRPNPQEFTYSSPTPDMEELQPVYV |
| | | NVGSVDVDVVYSQVWSMQQPESSANIRTLLENKDSQVIY |
| | | SSVKKS |
| FCRL3 | 125 | HYARARRKPGGLSATGTSSHSPSECQEPSSSRPSRIDPQEPT |
| | | HSKPLAPMELEPMYSNVNPGDSNPIYSQIWSIQHTKENSA |
| | | NCPMMHQEHEELTVLYSELKKTHPDDSAGEASSRGRAHE |
| | | EDDEENYENVPRVLLASDH |
| FCRL4 | 126 | HCWRRRKSGVGFLGDETRLPPAPGPGESSHSICPAQVELQ |
| | | SLYVDVHPKKGDLVYSEIQTTQLGEEEEANTSRTLLEDKD |
| | | VSVVYSEVKTQHPDNSAGKISSKDEES |
| FCRL5 | 127 | LSRKAGRKPASDPARSPSDSDSQEPTYHNVPAWEELQPVY |
| | | TNANPRGENVVYSEVRIIQEKKKHAVASDPRHLRNKGSPII |
| | | YSEVKVASTPVSGSLFLASSAPHR |
| SLAMF1 | 128 | QLRRRGKTNHYQTTVEKKSLTIYAQVQKPGPLQKKLDSFP |
| | | AQDPCTTIYVAATEPVPESVQETNSITVYASVTLPES |
| SLAMF5 | 129 | RLFKRRQGRIFPEGSCLNTFTKNPYAASKKTIYTYIMASRN |
| | | TQPAESRIYDEILQSKVLPSKEEPVNTVYSEVQFADKMGK |
| | | ASTQDSKPPGTSSYEIVI |
| BTLA | 130 | RRHQGKQNELSDTAGREINLVDAHLKSEQTEASTRQNSQ |
| | | VLLSETGIYDNDPDLCFRMQEGSEVYSNPCLEENKPGIVY |
| | | ASLNHSVIGPNSRLARNVKEAPTEYASICVRS |
| LAG3 | 131 | HLWRRQWRPRRFSALEQGIHPPQAQSKIEELEQEPEPEPEP |
| | | EPEPEPEPEPEQL |
| 2B4 | 132 | WRRKRKEKQSETSPKEFLTIYEDVKDLKTRRNHEQEQTFP |
| | | GGGSTIYSMIQSQSSAPTSQEPAYTLYSLIQPSRKSGSRKRN |
| | | HSPSFNSTIYEVIGKSQPKAQNPARLSRKELENFDVYS |
| CD160 | 133 | GCINITSSASQEGTRLNLICTVWHKKEEAEGFVVFLCKDRS |
| | | GDCSPETSLKQLRLKRDPGIDGVGEISSQLMFTISQVTPLH |
| | | SGTYQCCARSQKSGIRLQGHFFSILFTETGNYTVTGLKQR |
| | | QHLEFSHNEGTLS |
| CEACAM1 | 134 | HFGKTGRASDQRDLTEHKPSVSNHTQDHSNDPPNKMNEV |
| | | TYSTLNFEAQQPTQPTSASPSLTATEIIYSEVKKQ |
| TIM3 | 135 | FKWYSHSKEKIQNLSLISLANLPPSGLANAVAEGIRSEENI |
| | | YTIEENVYEVEEPNEYYCYVSSRQQPSQPLGCRFAMP |
| VISTA | 136 | YKQRQAASNRRAQELVRMDSNIQGIENPGFEASPPAQGIP |
| | | EAKVRHPLSYVAQRQPSESGRHLLSEPSTPLSPPGPGDVFF |
| | | PSLDPVPDSPNFEVI |
| TIGIT | 137 | LTRKKKALRIHSVEGDLRRKSAGQEEWSPSAPSPPGSCVQ |
| | | AEAAPAGLCGEQRGEDCAELHDYFNVLSYRSLGNCSFFTE |
| | | TG |
| SIRPalpha | 138 | RIRQKKAQGSTSSTRLHEPEKNAREITQDTNDITYADLNLP |
| | | KGKKPAPQAAEPNNHTEYASIQTSPQPASEDTLTYADLDM |
| | | VHLNRTPKQPAPKPEPSFSEYASVQVPRK |
| FcγRIIB | 139 | VVALIYCRKKRISALPGYPECREMGETLPEKPANPTNPDE |
| | | ADKVGAENTITYSLLMHPDALEEPDDQNRI |
| CD5 | 140 | KKLVKKFRQKKQRQWIGPTGMNQNMSFHRNHTATVRSH |
| | | AENPTASHVDNEYSQPPRNSHLSAYPALEGALHRSSMQPD |
| | | NSSDSDYDLHGAQRL |
| CD300a | 141 | RMFQKWIKAGDHSELSQNPKQAATQSELHYANLELLMW |
| | | PLQEKPAPPREVEVEYSTVASPREELHYASVVFDSNTNRIA |
| | | AQRPREEEPDSDYSVIRKT |
| CD300f | 142 | WRMMKYQQKAAGMSPEQVLQPLEGDLCYADLTLQLAG |
| | | TSPQKATTKLSSAQVDQVEVEYVTMASLPKEDISYASLTL |
| | | GAEDQEPTYCNMGHLSSHLPGRGPEEPTEYSTISRP |
| LIR1 | 143 | LRHRRQGKHWTSTQRKADFQHPAGAVGPEPTDRGLQWR |
| | | SSPAADAQEENLYAAVKHTQPEDGVEMDTRSPHDEDPQA |
| | | VTYAEVKHSRPRREMASPPSPLSGEFLDTKDRQAEEDRQ |
| | | MDTEAAASEAPQDVTYAQLHSLTLRREATEPPPSQEGPSP |
| | | AVPSIYATLAIH |
| LIR2 | 144 | LRHRRQGKHWTSTQRKADFQHPAGAVGPEPTDRGLQWR |
| | | SSPAADAQEENLYAAVKDTQPEDGVEMDTRAAASEAPQ |
| | | DVTYAQLHSLTLRRKATEPPPSQEREPPAEPSIYATLAIH |
| LIR3 | 145 | RRQRHSKHRTSDQRKTDFQRPAGAAETEPKDRGLLRRSSP |
| | | AADVQEENLYAAVKDTQSEDRVELDSQSPHDEDPQAVTY |
| | | APVKHSSPRREMASPPSSLSGEFLDTKDRQVEEDRQMDTE |
| | | AAASEASQDVTYAQLHSLTLRRKATEPPPSQEGEPPAEPSI |
| | | YATLAIH |
| LIR5 | 146 | QHWRQGKHRTLAQRQADFQRPPGAAEPEPKDGGLQRRSS |
| | | PAADVQGENFCAAVKNTQPEDGVEMDTRQSPHDEDPQA |
| | | VTYAKVKHSRPRREMASPPSPLSGEFLDTKDRQAEEDRQ |
| | | MDTEAAASEAPQDVTYAQLHSFTLRQKATEPPPSQEGASP |
| | | AEPSVYATLAIH |
| LIR8 | 147 | RHRHQSKHRTSAHFYRPAGAAGPEPKDQGLQKRASPVAD |
| | | IQEEILNAAVKDTQPKDGVEMDARAAASEAPQDVTYAQL |
| | | HSLTLRREATEPPPSQEREPPAEPSIYAPLAIH |
| Ly9 | 148 | KRKGRCSVPAFCSSQAEAPADTPEPTAGHTLYSVLSQGYE |
| | | KLDTPLRPARQQPTPTSDSSSDSNLTTEEDEDRPEVHKPIS |
| | | GRYEVFDQVTQEGAGHDPAPEGQADYDPVTPYVTEVESV |
| | | VGENTMYAQVFNLQGKTPVSQKEESSATIYCSIRKPQVVP |
| | | PPQQNDLEIPESPTYENFT |
| 2xPD1(G4S) | 149 | CSRAARGTIGARRTGQPLKEDPSAVPVFSVDYGELDFQW |
| | | REKTPEPPVPCVPEQTEYATIVFPSGMGTSSPARRGSADGP |
| | | RSAQPLRPEDGHCSWPLGGGGSGGGGSCSRAARGTIGAR |
| | | RTGQPLKEDPSAVPVFSVDYGELDFQWREKTPEPPVPCVP |
| | | EQTEYATIVFPSGMGTSSPARRGSADGPRSAQPLRPEDGH |
| | | CSWPL |
| 2xPD1(PD1) | 150 | CSRAARGTIGARRTGQPLKEDPSAVPVFSVDYGELDFQW |
| | | REKTPEPPVPCVPEQTEYATIDFQWREKTPEPPVPCVPEQV |
| | | DYGELDFQWREKTPEPPVPCVPEQTEYATIVFPSGMGTSS |
| | | PARRGSADGPRSAQPLRPEDGHCSWPL |
| PVRIg | 151 | LRRHKHRPAPRLQPSRTSPQAPRARAWAPSQASQAALHV |
| | | PYATINTSCRPATLDTAHPHGGPSWWASLPTHAAHRPQGP |
| | | AAWASTPIPARGSFVSVENGLYAQAGERPPHTGPGLTLFP |
| | | DPRGPRAMEGPLGVR |
| AA2AR | 152 | RIREFRQTFRKIIRSHVLRQQEPFKAAGTSARVLAAHGSDG |
| | | EQVSLRLNGHPPGVWANGSAPHPERRPNGYALGLVSGGS |
| | | AQESQGNTGLPDVELLSHELKGVCPEPPGLDDPLAQDGA |
| | | GVS |

### 5. Optional Synthetic PD-1 or LIR1 Sequences

In some embodiments, the iCAR construct comprises an optional synthetic PD-1 sequence. In some embodiments, the iCAR comprises a synthetic PD-1 sequence shown in Table 8. In some embodiments, the iCAR construct comprises an optional synthetic LIR1 sequence. In some embodiments, the iCAR comprises a synthetic LIR1 sequence shown in Table 8.

TABLE 8
Intracellular synthetic PD-1 and synthetic LIR1 sequences

| SEQ ID | |
|---|---|
| NO | Sequence |
| 243 | CSRAARGTIGARRTGQPLKEDPSAVPVFSVDYGELVFPSGMGTSSPARRGS |
| | ADGPRSAQPLRPEDGHCSWPL |
| 244 | CSRAARGTIGARRTGQPLKEDPSAVPVFSVDYGELDFQWREKTPEPPVPCV |
| | PEQVDYGELVFPSGMGTSSPARRGSADGPRSAQPLRPEDGHCSWPL |
| 245 | CSRAARGTIGARRTGQPLKEDPSAVPVFSVDYGELDFQWREKTPEPPVPCV |
| | PEQVDYGELDFQWREKTPEPPVPCVPEQVDYGELVFPSGMGTSSPARRGS |
| | ADGPRSAQPLRPEDGHCSWPL |
| 246 | CSRAARGTIGARRTGQPLKEDPSAVPVFSVDYGELDFQWREKTPEPPVPCV |
| | PEQVDYGELDFQWREKTPEPPVPCVPEQVDYGELDFQWREKTPEPPVPCV |
| | PEQVDYGELVFPSGMGTSSPARRGSADGPRSAQPLRPEDGHCSWPL |
| 247 | CSRAARGTIGARRTGQPLKEDPSAVPVFSVDYGELDFQWREKTPEPPVPCV |
| | PEQVDYGELDFQWREKTPEPPVPCVPEQVDYGELDFQWREKTPEPPVPCV |
| | PEQVDYGELDFQWREKTPEPPVPCVPEQVDYGELVFPSGMGTSSPARRGS |
| | ADGPRSAQPLRPEDGHCSWPL |
| 248 | CSRAARGTIGARRTGQPLKEDPSAVPVFSTEYATIVFPSGMGTSSPARRGSA |
| | DGPRSAQPLRPEDGHCSWPL |
| 249 | CSRAARGTIGARRTGQPLKEDPSAVPVFSTEYATIDFQWREKTPEPPVPCVP |
| | EQTEYATIVFPSGMGTSSPARRGSADGPRSAQPLRPEDGHCSWPL |
| 250 | CSRAARGTIGARRTGQPLKEDPSAVPVFSTEYATIDFQWREKTPEPPVPCVP |
| | EQTEYATIDFQWREKTPEPPVPCVPEQTEYATIVFPSGMGTSSPARRGSADG |
| | PRSAQPLRPEDGHCSWPL |
| 251 | CSRAARGTIGARRTGQPLKEDPSAVPVESTEYATIDFQWREKTPEPPVPCVP |
| | EQTEYATIDFQWREKTPEPPVPCVPEQTEYATIDFQWREKTPEPPVPCVPEQ |
| | TEYATIVFPSGMGTSSPARRGSADGPRSAQPLRPEDGHCSWPL |
| 252 | CSRAARGTIGARRTGQPLKEDPSAVPVFSTEYATIDFQWREKTPEPPVPCVP |
| | EQTEYATIDFQWREKTPEPPVPCVPEQTEYATIDFQWREKTPEPPVPCVPEQ |
| | TEYATIDFQWREKTPEPPVPCVPEQTEYATIVFPSGMGTSSPARRGSADGPR |
| | SAQPLRPEDGHCSWPL |
| 253 | CSRAARGTIGARRTGQPLKEDPSAVPVFSVDYGELDFQWREKTPEPPVPCV |
| | PEQTEYATIVFPSGMGTSSPARRGSADGPRSAQPLRPEDGHCSWPLGGGGS |
| | GGGGSCSRAARGTIGARRTGQPLKEDPSAVPVFSVDYGELDFQWREKTPEP |
| | PVPCVPEQTEYATIVFPSGMGTSSPARRGSADGPRSAQPLRPEDGHCSWPL |
| 254 | CSRAARGTIGARRTGQPLKEDPSAVPVFSVDYGELDFQWREKTPEPPVPCV |
| | PEQTEYATIDFQWREKTPEPPVPCVPEQVDYGELDFQWREKTPEPPVPCVP |
| | EQTEYATIVFPSGMGTSSPARRGSADGPRSAQPLRPEDGHCSWPL |
| 296 | LRHRRQGKHWTSTQRKADFQHPAGAVGPEPTDRGLQWRSSPAADAQEEN |
| | LYAAVKHTQPEDGVEMDTRSPHDEDPQANLYAAVKHSRPRREMASPPSPL |
| | SGEFLDTKDRQAEEDRQMDTEAAASEAPQDNLYAAVHSLTLRREATEPPP |
| | SQEGPSPAVPNLYAAVAIH |
| 297 | LRHRRQGKHWTSTQRKADFQHPAGAVGPEPTDRGLQWRSSPAADAQEEV |
| | TYAEVKHTQPEDGVEMDTRSPHDEDPQAVTYAEVKHSRPRREMASPPSPL |
| | SGEFLDTKDRQAEEDRQMDTEAAASEAPQDVTYAEVHSLTLRREATEPPPS |
| | QEGPSPAVPVTYAEVAIH |
| 298 | LRHRRQGKHWTSTQRKADFQHPAGAVGPEPTDRGLQWRSSPAADAQEEV |
| | TYAQLKHTQPEDGVEMDTRSPHDEDPQAVTYAQLKHSRPRREMASPPSPL |
| | SGEFLDTKDRQAEEDRQMDTEAAASEAPQDVTYAQLHSLTLRREATEPPPS |
| | QEGPSPAVPVTYAQLAIH |
| 299 | LRHRRQGKHWTSTQRKADFQHPAGAVGPEPTDRGLQWRSSPAADAQEESI |
| | YATLKHTQPEDGVEMDTRSPHDEDPQASIYATLKHSRPRREMASPPSPLSG |
| | EFLDTKDRQAEEDRQMDTEAAASEAPQDSIYATLHSLTLRREATEPPPSQE |
| | GPSPAVPSIYATLAIH |
| 300 | LRHRRQGKHWTSTQRKADFQHPAGAVGPEPTDRGLQWRSSPAADAQEEV |
| | TYAQLKHTQPEDGVEMDTRSPHDEDPQASIYATLKHSRPRREMASPPSPLS |
| | GEFLDTKDRQAEEDRQMDTEAAASEAPQDVTYAQLHSLTLRREATEPPPS |
| | QEGPSPAVPSIYATLAIH |
| 301 | LRHRRQGKHWTSTQRKADFQHPAGAVGPEPTDRGLQWRSSPAADAQEET |
| | EYATIKHTQPEDGVEMDTRSPHDEDPQATEYATIKHSRPRREMASPPSPLS |
| | GEFLDTKDRQAEEDRQMDTEAAASEAPQDVTYAQLHSLTLRREATEPPPS |
| | QEGPSPAVPSIYATLAIH |
| 302 | LRHRRQGKHWTSTQRKADFQHPAGAVGPEPTDRGLQWRSSPAADAQEEV |
| | TYAQLKHTQPEDGVEMDTRSPHDEDPQASIYATLKHSRPRREMASPPSPLS |
| | GEFLDTKDRQAEEDRQMDTEAAASEAPQDTEYATIHSLTLRREATEPPPSQ |
| | EGPSPAVPTEYATIAIH |
| 304 | LRHRRQGKHWTSTQRKADFQHPAGAVGPEPTDRGLQWRSSPAADAQEEV |
| | TYAQLKHTQPEDGVEMDTRSPHDEDPQATEYATIKHSRPRREMASPPSPLS |
| | GEFLDTKDRQAEEDRQMDTEAAASEAPQDTEYATIHSLTLRREATEPPPSQ |
| | EGPSPAVPSIYATLAIH |

6. Exemplary iCARs

In some embodiments, the iCAR comprises an scFv component comprising the VL and VH sequences of BB7.2 (SEQ ID NOs: 37 and 38). In some embodiments, the orientation of the iCAR VH and VL regions is VH-VL. In some embodiments, the orientation of the iCAR VH and VL regions is VL-VH. In some embodiments, the iCAR scFv comprises a (G₄S)X3 linker (SEQ ID NO:81) linker that covalently connects the VH and the VL to form the iCAR scFv. In some embodiments, the iCAR scFv comprises a Whitlow linker (SEQ ID NO:82) linker that covalently connects the VH and the VL to form the iCAR scFv. In some embodiments, the iCAR comprises a CD8 alpha hinge domain (SEQ ID NO:84). In some embodiments, the iCAR comprises a CD28 hinge domain (SEQ ID NO:85). In some embodiments, the iCAR comprises a PD-1 hinge domain (SEQ ID NO:86). In some embodiments, the iCAR comprises a LIR1 Ig3-4 hinge domain (SEQ ID NO:87). In some embodiments, the iCAR comprises a LIR1 Ig-4 hinge domain (SEQ ID NO:88). In some embodiments, the iCAR comprises a LIR1 52 aa hinge domain (SEQ ID NO:89). In some embodiments, the iCAR comprises a LIR1 36 aa hinge domain (SEQ ID NO:90). In some embodiments, the iCAR comprises a LIR1 30 aa hinge domain (SEQ ID NO:91). In some embodiments, the iCAR comprises a LIR1 8 aa hinge domain (SEQ ID NO:92). In some embodiments, the iCAR comprises a CD33 hinge domain (SEQ ID NO:93). In some embodiments, the iCAR comprises a KIR2DL1 hinge domain (SEQ ID NO:94). In some embodiments, the iCAR comprises a LIR1 26 aa (SEQ ID NO: 289). In some embodiments, the iCAR comprises PD-1 (47) (SEQ ID NO: 290). In some embodiments, the iCAR comprises PD-1 (42) (SEQ ID NO: 291). In some embodiments, the iCAR comprises PD-1 (36) (SEQ ID NO: 292). In some embodiments, the iCAR comprises PD-1 (30) (SEQ ID NO: 293). In some embodiments, the iCAR comprises PD-1 (26) (SEQ ID NO: 294). In some embodiments, the iCAR comprises PD-1 (20) (SEQ ID NO: 295). In some embodiments, the iCAR comprises a CD8 alpha transmembrane domain (SEQ ID NO:95). In some embodiments, the iCAR comprises a CD28 transmembrane domain (SEQ ID NO:96). In some embodiments, the iCAR comprises a PD-1 transmembrane domain (SEQ ID NO:97). In some embodiments, the iCAR comprises a LIR1 transmembrane domain (SEQ ID NO:98). In some embodiments, the iCAR comprises a CD33 transmembrane domain (SEQ ID NO:99). In some embodiments, the iCAR comprises a KIR2DL1 transmembrane domain (SEQ ID NO:100). In some embodiments, the iCAR comprises a PD-1 inhibitory domain (SEQ ID NO:101). In some embodiments, the iCAR comprises a KIR2DL1 inhibitory domain (SEQ ID NO:102). In some embodiments, the iCAR comprises a KIR2DL2 inhibitory domain (SEQ ID NO:103). In some embodiments, the iCAR comprises a KIR2DL3 inhibitory domain (SEQ ID NO:104). In some embodiments, the iCAR comprises a KIR2DL4 inhibitory domain (SEQ ID NO:105). In some embodiments, the iCAR comprises a KIR2DL5A inhibitory domain (SEQ ID NO:106). In some embodiments, the iCAR comprises a KIR3DL1 inhibitory domain (SEQ ID NO:107). In some embodiments, the iCAR comprises a KIR3DL2 inhibitory domain (SEQ ID NO:108). In some embodiments, the iCAR comprises a KIR3DL3 inhibitory domain (SEQ ID NO:109). In some embodiments, the iCAR comprises a LAIR1 inhibitory domain (SEQ ID NO:110). In some embodiments, the iCAR comprises a CD22 inhibitory domain (SEQ ID NO:111). In some embodiments, the iCAR comprises a CD33 inhibitory domain (SEQ ID NO:112). In some embodiments, the iCAR comprises a SIGLEC5 inhibitory domain (SEQ ID NO:113). In some embodiments, the iCAR comprises a SIGLEC6 inhibitory domain (SEQ ID NO:114). In some embodiments, the iCAR comprises a SIGLEC7 inhibitory domain (SEQ ID NO:115). In some embodiments, the iCAR comprises a SIGLEC8 inhibitory domain (SEQ ID NO:116). In some embodiments, the iCAR comprises a SIGLEC9 inhibitory domain (SEQ ID NO:117). In some embodiments, the iCAR comprises a SIGLEC10 inhibitory domain (SEQ ID NO:118). In some embodiments, the iCAR comprises a SIGLEC11 inhibitory domain (SEQ ID NO:119). In some embodiments, the iCAR comprises a SIGLEC12 inhibitory domain (SEQ ID NO:120). In some embodiments, the iCAR comprises a PECAM1/CD31 inhibitory domain (SEQ ID NO:121). In some embodiments, the iCAR comprises a CD200R1 inhibitory domain (SEQ ID NO:]122). In some embodiments, the iCAR comprises a FCRL1 inhibitory domain (SEQ ID NO:123). In some embodiments, the iCAR comprises a FCRL2 inhibitory domain (SEQ ID NO:124). In some embodiments, the iCAR comprises a FCRL3 inhibitory domain (SEQ ID NO:125). In some embodiments, the iCAR comprises a FCRL4 inhibitory domain (SEQ ID NO:126). In some embodiments, the iCAR comprises a FCRL5 inhibitory domain (SEQ ID NO:127). In some embodiments, the iCAR comprises a SLAMF1 inhibitory domain (SEQ ID NO:128). In some embodiments, the iCAR comprises a SLAMF5 inhibitory domain (SEQ ID NO:129). In some embodiments, the iCAR comprises a BTLA inhibitory domain (SEQ ID NO:130). In some embodiments, the iCAR comprises a LAG3 inhibitory domain (SEQ ID NO:131). In some embodiments, the iCAR comprises a 2B4 inhibitory domain (SEQ ID NO:132). In some embodiments, the iCAR comprises a CD160 inhibitory domain (SEQ ID NO:133). In some embodiments, the iCAR comprises a CEACAM1 inhibitory domain (SEQ ID NO:134). In some embodiments, the iCAR comprises a TIM3 inhibitory domain (SEQ ID NO:135). In some embodiments, the iCAR comprises a VISTA inhibitory domain (SEQ ID NO:136). In some embodiments, the iCAR comprises a TIGIT inhibitory domain (SEQ ID NO:137). In some embodiments, the iCAR comprises a SIRPalpha inhibitory domain (SEQ ID NO:138). In some embodiments, the iCAR comprises a FcγRIIB inhibitory domain (SEQ ID NO:139). In some embodiments, the iCAR comprises a CD5 inhibitory domain (SEQ ID NO:140). In some embodiments, the iCAR comprises a CD300a inhibitory domain (SEQ ID NO:141). In some embodiments, the iCAR comprises a CD300f inhibitory domain (SEQ ID NO:142). In some embodiments, the iCAR comprises a LIR1 inhibitory domain (SEQ ID NO:143). In some embodiments, the iCAR comprises a LIR2 inhibitory domain (SEQ ID NO:144). In some embodiments, the iCAR comprises a LIR3 inhibitory domain (SEQ ID NO:145). In some embodiments, the iCAR comprises a LIR5 inhibitory domain (SEQ ID NO:146). In some embodiments, the iCAR comprises a LIR8 inhibitory domain (SEQ ID NO:147). In some embodiments, the iCAR comprises a Ly9 inhibitory domain (SEQ ID NO:148). In some embodiments, the iCAR comprises a 2×PD1(G4S) inhibitory domain (SEQ ID NO:149). In some embodiments, the iCAR comprises a 2×PD1(PD1) inhibitory domain (SEQ ID NO:150). In some embodiments, the iCAR comprises a PVRIg inhibitory domain (SEQ ID NO:151). In some embodiments, the iCAR comprises an AA2AR inhibitory domain (SEQ ID NO:152). In some embodiments, the iCAR comprises a signal peptide upstream of the iCAR portion, wherein the signal peptide is a CD8 alpha signal peptide (SEQ ID NO: 161), a GM-CSF signal peptide (SEQ ID NO: 162), or a mIgK signal peptide (SEQ ID NO: 306). In some embodiments, the iCAR comprises an scFv component comprising the VL and VH sequences of 3PF12/C4 (SEQ ID NOs: 39 and 40). In some embodiments, the orientation of the iCAR VH and VL regions is VH-VL. In some embodiments, the orientation of the iCAR VH and VL regions is VL-VH. In some embodiments, the iCAR scFv comprises a (G4S)X3 linker (SEQ ID NO:81) linker that covalently connects the VH and the VL to form the iCAR scFv. In some embodiments, the iCAR scFv comprises a Whitlow linker (SEQ ID NO:82) linker that covalently connects the VH and the VL to form the iCAR scFv. In some embodiments, the iCAR comprises a CD8 alpha hinge domain (SEQ ID NO:84). In some embodiments, the iCAR comprises a CD28 hinge domain (SEQ ID NO:85). In some embodiments, the iCAR comprises a PD-1 hinge domain (SEQ ID NO:86). In some embodiments, the iCAR comprises a LIR1 Ig3-4 hinge domain (SEQ ID NO:87). In some embodiments, the iCAR comprises a LIR1 Ig-4 hinge domain (SEQ ID NO:88). In some embodiments, the iCAR comprises a LIR1 52 aa hinge domain (SEQ ID NO:89). In some embodiments, the iCAR comprises a LIR1 36 aa hinge domain (SEQ ID NO:90). In some embodiments, the iCAR comprises a LIR1 30 aa hinge domain (SEQ ID NO:91). In some embodiments, the iCAR comprises a LIR1 8 aa hinge domain (SEQ ID NO:92). In some embodiments, the iCAR comprises a CD33 hinge domain (SEQ ID NO:93). In some embodiments, the iCAR comprises a KIR2DL1 hinge domain (SEQ ID NO:94). In some embodiments, the iCAR comprises a LIR1 26 aa hinge domain (SEQ ID NO: 289). In some embodiments, the iCAR comprises PD-1 (47) hinge domain (SEQ ID NO: 290). In some embodiments, the iCAR comprises PD-1 (42) hinge domain (SEQ ID NO: 291). In some embodiments, the iCAR comprises PD-1 (36) hinge domain (SEQ ID NO: 292). In some embodiments, the iCAR comprises PD-1 (30) hinge domain (SEQ ID NO: 293). In some embodiments, the iCAR comprises PD-1 (26) hinge domain (SEQ ID NO: 294). In some embodiments, the iCAR comprises PD-1 (20) hinge domain (SEQ ID NO: 295). In some embodiments, the iCAR comprises a CD8 alpha transmembrane domain (SEQ ID NO:95). In some embodiments, the iCAR comprises a CD28 transmembrane domain (SEQ ID NO:96). In some embodiments, the iCAR comprises a PD-1 transmembrane domain (SEQ ID NO:97). In some embodiments, the iCAR comprises a LIR1 transmembrane domain (SEQ ID NO:98). In some embodiments, the iCAR comprises a CD33 transmembrane domain (SEQ ID NO:99). In some embodiments, the iCAR comprises a KIR2DL1 transmembrane domain (SEQ ID NO:100). In some embodiments, the iCAR comprises a PD-1 inhibitory domain (SEQ ID NO:101). In some embodiments, the iCAR comprises a KIR2DL1 inhibitory domain (SEQ ID NO:102). In some embodiments, the iCAR comprises a KIR2DL2 inhibitory domain (SEQ ID NO:103). In some embodiments, the iCAR comprises a KIR2DL3 inhibitory domain (SEQ ID NO:104). In some embodiments, the iCAR comprises a KIR2DL4 inhibitory domain (SEQ ID NO:105). In some embodiments, the iCAR comprises a KIR2DL5A inhibitory domain (SEQ ID NO:106). In some embodiments, the iCAR comprises a KIR3DL1 inhibitory domain (SEQ ID NO:107). In some embodiments, the iCAR comprises a KIR3DL2 inhibitory domain (SEQ ID NO:108). In some embodiments, the iCAR comprises a KIR3DL3 inhibitory domain (SEQ ID NO:109). In some embodiments, the iCAR comprises a LAIR1 inhibitory domain (SEQ ID NO:110). In some embodiments, the iCAR comprises a CD22 inhibitory domain (SEQ ID NO:111). In some embodiments, the iCAR comprises a CD33 inhibitory domain (SEQ ID NO:112). In some embodiments, the iCAR comprises a SIGLEC5 inhibitory domain (SEQ ID NO:113). In some embodiments, the iCAR comprises a SIGLEC6 inhibitory domain (SEQ ID NO:114). In some embodiments, the iCAR comprises a SIGLEC7 inhibitory domain (SEQ ID NO:115). In some embodiments, the iCAR comprises a SIGLEC8 inhibitory domain (SEQ ID NO:116). In some embodiments, the iCAR comprises a SIGLEC9 inhibitory domain (SEQ ID NO:117). In some embodiments, the iCAR comprises a SIGLEC10 inhibitory domain (SEQ ID NO:118). In some embodiments, the iCAR comprises a SIGLEC11 inhibitory domain (SEQ ID NO:119). In some embodiments, the iCAR comprises a SIGLEC12 inhibitory domain (SEQ ID NO:120). In some embodiments, the iCAR comprises a PECAM1/CD31 inhibitory domain (SEQ ID NO:121). In some embodiments, the iCAR comprises a CD200R1 inhibitory domain (SEQ ID NO:122). In some embodiments, the iCAR comprises a FCRL1 inhibitory domain (SEQ ID NO:123). In some embodiments, the iCAR comprises a FCRL2 inhibitory domain (SEQ ID NO:124). In some embodiments, the iCAR comprises a FCRL3 inhibitory domain (SEQ ID NO:125). In some embodiments, the iCAR comprises a FCRL4 inhibitory domain (SEQ ID NO:126). In some embodiments, the iCAR comprises a FCRL5 inhibitory domain (SEQ ID NO:127). In some embodiments, the iCAR comprises a SLAMF1 inhibitory domain (SEQ ID NO:128). In some embodiments, the iCAR comprises a SLAMF5 inhibitory domain (SEQ ID NO:129). In some embodiments, the iCAR comprises a BTLA inhibitory domain (SEQ ID NO:130). In some embodiments, the iCAR comprises a LAG3 inhibitory domain (SEQ ID NO:131). In some embodiments, the iCAR comprises a 2B4 inhibitory domain (SEQ ID NO:132). In some embodiments, the iCAR comprises a CD160 inhibitory domain (SEQ ID NO:133). In some embodiments, the iCAR comprises a CEACAM1 inhibitory domain (SEQ ID NO:134). In some embodiments, the iCAR comprises a TIM3 inhibitory domain (SEQ ID NO:135). In some embodiments, the iCAR comprises a VISTA inhibitory domain (SEQ ID NO:136). In some embodiments, the iCAR comprises a TIGIT inhibitory domain (SEQ ID NO:137). In some embodiments, the iCAR comprises a SIRPalpha inhibitory domain (SEQ ID NO:138). In some embodiments, the iCAR comprises a FcγRIIB inhibitory domain (SEQ ID NO:139). In some embodiments, the iCAR comprises a CD5 inhibitory domain (SEQ ID NO:140). In some embodiments, the iCAR comprises a CD300a inhibitory domain (SEQ ID NO:141). In some embodiments, the iCAR comprises a CD300f inhibitory domain (SEQ ID NO:142). In some embodiments, the iCAR comprises a LIR1 inhibitory domain (SEQ ID NO:143). In some embodiments, the iCAR comprises a LIR2 inhibitory domain (SEQ ID NO:144). In some embodiments, the iCAR comprises a LIR3 inhibitory domain (SEQ ID NO:145). In some embodiments, the iCAR comprises a LIR5 inhibitory domain (SEQ ID NO:146). In some embodiments, the iCAR comprises a LIR8 inhibitory domain (SEQ ID NO:147). In some embodiments, the iCAR comprises a Ly9 inhibitory domain (SEQ ID NO:148). In some embodiments, the iCAR comprises a 2×PD1(G4S) inhibitory domain (SEQ ID NO:149). In some embodiments, the iCAR comprises a 2×PD1(PD1) inhibitory domain (SEQ ID NO:150). In some embodiments, the iCAR comprises a PVRIg inhibitory domain (SEQ ID NO:151). In some embodiments, the iCAR comprises an AA2AR inhibitory domain (SEQ ID NO:152). In some embodiments, the iCAR comprises a signal peptide upstream of the iCAR portion, wherein the signal peptide is a CD8 alpha signal peptide (SEQ ID NO: 161), a GM-CSF signal peptide (SEQ ID NO: 162), or a mIgK signal peptide (SEQ ID NO: 306).

In some embodiments, the iCAR comprises an scFv component comprising the VL and VH sequences of 3PF12/F12 (SEQ ID NOs: 41 and 42). In some embodiments, the orientation of the iCAR VH and VL regions is VH-VL. In some embodiments, the orientation of the iCAR VH and VL regions is VL-VH. In some embodiments, the iCAR scFv comprises a (G4S)X3 linker (SEQ ID NO:81) linker that covalently connects the VH and the VL to form the iCAR scFv. In some embodiments, the iCAR scFv comprises a Whitlow linker (SEQ ID NO:82) linker that covalently connects the VH and the VL to form the iCAR scFv. In some embodiments, the iCAR comprises a CD8 alpha hinge domain (SEQ ID NO:84). In some embodiments, the iCAR comprises a CD28 hinge domain (SEQ ID NO:85). In some embodiments, the iCAR comprises a PD-1 hinge domain (SEQ ID NO:86). In some embodiments, the iCAR comprises a LIR1 Ig3-4 hinge domain (SEQ ID NO:87). In some embodiments, the iCAR comprises a LIR1 Ig-4 hinge domain (SEQ ID NO:88). In some embodiments, the iCAR comprises a LIR1 52 aa hinge domain (SEQ ID NO:89). In some embodiments, the iCAR comprises a LIR1 36 aa hinge domain (SEQ ID NO:90). In some embodiments, the iCAR comprises a LIR1 30 aa hinge domain (SEQ ID NO:91). In some embodiments, the iCAR comprises a LIR1 8 aa hinge domain (SEQ ID NO:92). In some embodiments, the iCAR comprises a CD33 hinge domain (SEQ ID NO:93). In some embodiments, the iCAR comprises a KIR2DL1 hinge domain (SEQ ID NO:94). In some embodiments, the iCAR comprises a LIR1 26 aa hinge domain (SEQ ID NO: 289). In some embodiments, the iCAR comprises PD-1 (47) hinge domain (SEQ ID NO: 290). In some embodiments, the iCAR comprises PD-1 (42) hinge domain (SEQ ID NO: 291). In some embodiments, the iCAR comprises PD-1 (36) hinge domain (SEQ ID NO: 292). In some embodiments, the iCAR comprises PD-1 (30) hinge domain (SEQ ID NO: 293). In some embodiments, the iCAR comprises PD-1 (26) hinge domain (SEQ ID NO: 294). In some embodiments, the iCAR comprises PD-1 (20) hinge domain (SEQ ID NO: 295). In some embodiments, the iCAR comprises a CD8 alpha transmembrane domain (SEQ ID NO:95). In some embodiments, the iCAR comprises a CD28 transmembrane domain (SEQ ID NO:96). In some embodiments, the iCAR comprises a PD-1 transmembrane domain (SEQ ID NO:97). In some embodiments, the iCAR comprises a LIR1 transmembrane domain (SEQ ID NO:98). In some embodiments, the iCAR comprises a CD33 transmembrane domain (SEQ ID NO:99). In some embodiments, the iCAR comprises a KIR2DL1 transmembrane domain (SEQ ID NO:100). In some embodiments, the iCAR comprises a PD-1 inhibitory domain (SEQ ID NO:101). In some embodiments, the iCAR comprises a KIR2DL1 inhibitory domain (SEQ ID NO:102). In some embodiments, the iCAR comprises a KIR2DL2 inhibitory domain (SEQ ID NO:103). In some embodiments, the iCAR comprises a KIR2DL3 inhibitory domain (SEQ ID NO:104). In some embodiments, the iCAR comprises a KIR2DL4 inhibitory domain (SEQ ID NO:105). In some embodiments, the iCAR comprises a KIR2DL5A inhibitory domain (SEQ ID NO:106). In some embodiments, the iCAR comprises a KIR3DL1 inhibitory domain (SEQ ID NO:107). In some embodiments, the iCAR comprises a KIR3DL2 inhibitory domain (SEQ ID NO:108). In some embodiments, the iCAR comprises a KIR3DL3 inhibitory domain (SEQ ID NO:109). In some embodiments, the iCAR comprises a LAIR1 inhibitory domain (SEQ ID NO:110). In some embodiments, the iCAR comprises a CD22 inhibitory domain (SEQ ID NO:111). In some embodiments, the iCAR comprises a CD33 inhibitory domain (SEQ ID NO:112). In some embodiments, the iCAR comprises a SIGLEC5 inhibitory domain (SEQ ID NO:113). In some embodiments, the iCAR comprises a SIGLEC6 inhibitory domain (SEQ ID NO:114). In some embodiments, the iCAR comprises a SIGLEC7 inhibitory domain (SEQ ID NO:115). In some embodiments, the iCAR comprises a SIGLEC8 inhibitory domain (SEQ ID NO:116). In some embodiments, the iCAR comprises a SIGLEC9 inhibitory domain (SEQ ID NO:117). In some embodiments, the iCAR comprises a SIGLEC10 inhibitory domain (SEQ ID NO:118). In some embodiments, the iCAR comprises a SIGLEC11 inhibitory domain (SEQ ID NO:119). In some embodiments, the iCAR comprises a SIGLEC12 inhibitory domain (SEQ ID NO:120). In some embodiments, the iCAR comprises a PECAM1/CD31 inhibitory domain (SEQ ID NO:121). In some embodiments, the iCAR comprises a CD200R1 inhibitory domain (SEQ ID NO:122). In some embodiments, the iCAR comprises a FCRL1 inhibitory domain (SEQ ID NO:123). In some embodiments, the iCAR comprises a FCRL2 inhibitory domain (SEQ ID NO:124). In some embodiments, the iCAR comprises a FCRL3 inhibitory domain (SEQ ID NO:125). In some embodiments, the iCAR comprises a FCRL4 inhibitory domain (SEQ ID NO:126). In some embodiments, the iCAR comprises a FCRL5 inhibitory domain (SEQ ID NO:127). In some embodiments, the iCAR comprises a SLAMF1 inhibitory domain (SEQ ID NO:128). In some embodiments, the iCAR comprises a SLAMF5 inhibitory domain (SEQ ID NO:129). In some embodiments, the iCAR comprises a BTLA inhibitory domain (SEQ ID NO:130). In some embodiments, the iCAR comprises a LAG3 inhibitory domain (SEQ ID NO:131). In some embodiments, the iCAR comprises a 2B4 inhibitory domain (SEQ ID NO:132). In some embodiments, the iCAR comprises a CD160 inhibitory domain (SEQ ID NO:133). In some embodiments, the iCAR comprises a CEACAM1 inhibitory domain (SEQ ID NO:134). In some embodiments, the iCAR comprises a TIM3 inhibitory domain (SEQ ID NO:135). In some embodiments, the iCAR comprises a VISTA inhibitory domain (SEQ ID NO:136). In some embodiments, the iCAR comprises a TIGIT inhibitory domain (SEQ ID NO:137). In some embodiments, the iCAR comprises a SIRPalpha inhibitory domain (SEQ ID NO:138). In some embodiments, the iCAR comprises a FcγRIIB inhibitory domain (SEQ ID NO:139). In some embodiments, the iCAR comprises a CD5 inhibitory domain (SEQ ID NO:140). In some embodiments, the iCAR comprises a CD300a inhibitory domain (SEQ ID NO:141). In some embodiments, the iCAR comprises a CD300f inhibitory domain (SEQ ID NO:142). In some embodiments, the iCAR comprises a LIR1 inhibitory domain (SEQ ID NO:143). In some embodiments, the iCAR comprises a LIR2 inhibitory domain (SEQ ID NO:144). In some embodiments, the iCAR comprises a LIR3 inhibitory domain (SEQ ID NO:145). In some embodiments, the iCAR comprises a LIR5 inhibitory domain (SEQ ID NO:146). In some embodiments, the iCAR comprises a LIR8 inhibitory domain (SEQ ID NO:147). In some embodiments, the iCAR comprises a Ly9 inhibitory domain (SEQ ID NO:148). In some embodiments, the iCAR comprises a 2×PD1(G4S) inhibitory domain (SEQ ID NO:149). In some embodiments, the iCAR comprises a 2×PD1(PD1) inhibitory domain (SEQ ID NO:150). In some embodiments, the iCAR comprises a PVRIg inhibitory domain (SEQ ID NO:151). In some embodiments, the iCAR comprises an AA2AR inhibitory domain (SEQ ID NO:152). In some embodiments, the iCAR comprises a signal peptide upstream of the iCAR portion, wherein the signal peptide is a CD8 alpha signal peptide (SEQ ID NO: 161), a GM-CSF signal peptide (SEQ ID NO: 162), or a mIgK signal peptide (SEQ ID NO: 306).

In some embodiments, the iCAR comprises an scFv component comprising the VL and VH sequences of 3PF12/B11 (SEQ ID NOs: 43 and 44). In some embodiments, the orientation of the iCAR VH and VL regions is VH-VL. In some embodiments, the orientation of the iCAR VH and VL regions is VL-VH. In some embodiments, the iCAR scFv comprises a (G4S)X3 linker (SEQ ID NO:81) linker that covalently connects the VH and the VL to form the iCAR scFv. In some embodiments, the iCAR scFv comprises a Whitlow linker (SEQ ID NO:82) linker that covalently connects the VH and the VL to form the iCAR scFv. In some embodiments, the iCAR comprises a CD8 alpha hinge domain (SEQ ID NO:84). In some embodiments, the iCAR comprises a CD28 hinge domain (SEQ ID NO:85). In some embodiments, the iCAR comprises a PD-1 hinge domain (SEQ ID NO:86). In some embodiments, the iCAR comprises a LIR1 Ig3-4 hinge domain (SEQ ID NO:87). In some embodiments, the iCAR comprises a LIR1 Ig-4 hinge domain (SEQ ID NO:88). In some embodiments, the iCAR comprises a LIR1 52 aa hinge domain (SEQ ID NO:89). In some embodiments, the iCAR comprises a LIR1 36 aa hinge domain (SEQ ID NO:90). In some embodiments, the iCAR comprises a LIR1 30 aa hinge domain (SEQ ID NO:91). In some embodiments, the iCAR comprises a LIR1 8 aa hinge domain (SEQ ID NO:92). In some embodiments, the iCAR comprises a CD33 hinge domain (SEQ ID NO:93). In some embodiments, the iCAR comprises a KIR2DL1 hinge domain (SEQ ID NO:94). In some embodiments, the iCAR comprises a LIR1 26 aa hinge domain (SEQ ID NO: 289). In some embodiments, the iCAR comprises PD-1 (47) hinge domain (SEQ ID NO: 290). In some embodiments, the iCAR comprises PD-1 (42) hinge domain (SEQ ID NO: 291). In some embodiments, the iCAR comprises PD-1 (36) hinge domain (SEQ ID NO: 292). In some embodiments, the iCAR comprises PD-1 (30) hinge domain (SEQ ID NO: 293). In some embodiments, the iCAR comprises PD-1 (26) hinge domain (SEQ ID NO: 294). In some embodiments, the iCAR comprises PD-1 (20) hinge domain (SEQ ID NO: 295). In some embodiments, the iCAR comprises a CD8 alpha transmembrane domain (SEQ ID NO:95). In some embodiments, the iCAR comprises a CD28 transmembrane domain (SEQ ID NO:96). In some embodiments, the iCAR comprises a PD-1 transmembrane domain (SEQ ID NO:97). In some embodiments, the iCAR comprises a LIR1 transmembrane domain (SEQ ID NO:98). In some embodiments, the iCAR comprises a CD33 transmembrane domain (SEQ ID NO:99). In some embodiments, the iCAR comprises a KIR2DL1 transmembrane domain (SEQ ID NO:100). In some embodiments, the iCAR comprises a PD-1 inhibitory domain (SEQ ID NO:101). In some embodiments, the iCAR comprises a KIR2DL1 inhibitory domain (SEQ ID NO:102). In some embodiments, the iCAR comprises a KIR2DL2 inhibitory domain (SEQ ID NO:103). In some embodiments, the iCAR comprises a KIR2DL3 inhibitory domain (SEQ ID NO:104). In some embodiments, the iCAR comprises a KIR2DL4 inhibitory domain (SEQ ID NO:105). In some embodiments, the iCAR comprises a KIR2DL5A inhibitory domain (SEQ ID NO:106). In some embodiments, the iCAR comprises a KIR3DL1 inhibitory domain (SEQ ID NO:107). In some embodiments, the iCAR comprises a KIR3DL2 inhibitory domain (SEQ ID NO:108). In some embodiments, the iCAR comprises a KIR3DL3 inhibitory domain (SEQ ID NO:109). In some embodiments, the iCAR comprises a LAIR1 inhibitory domain (SEQ ID NO:110). In some embodiments, the iCAR comprises a CD22 inhibitory domain (SEQ ID NO:111). In some embodiments, the iCAR comprises a CD33 inhibitory domain (SEQ ID NO:112). In some embodiments, the iCAR comprises a SIGLEC5 inhibitory domain (SEQ ID NO:113). In some embodiments, the iCAR comprises a SIGLEC6 inhibitory domain (SEQ ID NO:114). In some embodiments, the iCAR comprises a SIGLEC7 inhibitory domain (SEQ ID NO:115). In some embodiments, the iCAR comprises a SIGLEC8 inhibitory domain (SEQ ID NO:116). In some embodiments, the iCAR comprises a SIGLEC9 inhibitory domain (SEQ ID NO:117). In some embodiments, the iCAR comprises a SIGLEC10 inhibitory domain (SEQ ID NO:118). In some embodiments, the iCAR comprises a SIGLEC11 inhibitory domain (SEQ ID NO:119). In some embodiments, the iCAR comprises a SIGLEC12 inhibitory domain (SEQ ID NO:120). In some embodiments, the iCAR comprises a PECAM1/CD31 inhibitory domain (SEQ ID NO:121). In some embodiments, the iCAR comprises a CD200R1 inhibitory domain (SEQ ID NO:122). In some embodiments, the iCAR comprises a FCRL1 inhibitory domain (SEQ ID NO:123). In some embodiments, the iCAR comprises a FCRL2 inhibitory domain (SEQ ID NO:124). In some embodiments, the iCAR comprises a FCRL3 inhibitory domain (SEQ ID NO:125). In some embodiments, the iCAR comprises a FCRL4 inhibitory domain (SEQ ID NO:126). In some embodiments, the iCAR comprises a FCRL5 inhibitory domain (SEQ ID NO:127). In some embodiments, the iCAR comprises a SLAMF1 inhibitory domain (SEQ ID NO:128). In some embodiments, the iCAR comprises a SLAMF5 inhibitory domain (SEQ ID NO:129). In some embodiments, the iCAR comprises a BTLA inhibitory domain (SEQ ID NO:130). In some embodiments, the iCAR comprises a LAG3 inhibitory domain (SEQ ID NO:131). In some embodiments, the iCAR comprises a 2B4 inhibitory domain (SEQ ID NO:132). In some embodiments, the iCAR comprises a CD160 inhibitory domain (SEQ ID NO:133). In some embodiments, the iCAR comprises a CEACAM1 inhibitory domain (SEQ ID NO:134). In some embodiments, the iCAR comprises a TIM3 inhibitory domain (SEQ ID NO:135). In some embodiments, the iCAR comprises a VISTA inhibitory domain (SEQ ID NO:136). In some embodiments, the iCAR comprises a TIGIT inhibitory domain (SEQ ID NO:137). In some embodiments, the iCAR comprises a SIRPalpha inhibitory domain (SEQ ID NO:138). In some embodiments, the iCAR comprises a FcγRIIB inhibitory domain (SEQ ID NO:139). In some embodiments, the iCAR comprises a CD5 inhibitory domain (SEQ ID NO:140). In some embodiments, the iCAR comprises a CD300a inhibitory domain (SEQ ID NO:141). In some embodiments, the iCAR comprises a CD300f inhibitory domain (SEQ ID NO:142). In some embodiments, the iCAR comprises a LIR1 inhibitory domain (SEQ ID NO:143). In some embodiments, the iCAR comprises a LIR2 inhibitory domain (SEQ ID NO:144). In some embodiments, the iCAR comprises a LIR3 inhibitory domain (SEQ ID NO:145). In some embodiments, the iCAR comprises a LIR5 inhibitory domain (SEQ ID NO:146). In some embodiments, the iCAR comprises a LIR8 inhibitory domain (SEQ ID NO:147). In some embodiments, the iCAR comprises a Ly9 inhibitory domain (SEQ ID NO:148). In some embodiments, the iCAR comprises a 2×PD1(G4S) inhibitory domain (SEQ ID NO:149). In some embodiments, the iCAR comprises a 2×PD1(PD1) inhibitory domain (SEQ ID NO:150). In some embodiments, the iCAR comprises a PVRIg inhibitory domain (SEQ ID NO:151). In some embodiments, the iCAR comprises an AA2AR inhibitory domain (SEQ ID NO:152). In some embodiments, the iCAR comprises a signal peptide upstream of the iCAR portion, wherein the signal peptide is a CD8 alpha signal peptide (SEQ ID NO: 161), a GM-CSF signal peptide (SEQ ID NO: 162), or a mIgK signal peptide (SEQ ID NO: 306).

In some embodiments, the iCAR comprises an scFv component comprising the VL and VH sequences of W6/32 (SEQ ID NOs: 45 and 46). In some embodiments, the orientation of the iCAR VH and VL regions is VH-VL. In some embodiments, the orientation of the iCAR VH and VL regions is VL-VH. In some embodiments, the iCAR scFv comprises a (G4S)X3 linker (SEQ ID NO:81) linker that covalently connects the VH and the VL to form the iCAR scFv. In some embodiments, the iCAR scFv comprises a Whitlow linker (SEQ ID NO:82) linker that covalently connects the VH and the VL to form the iCAR scFv. In some embodiments, the iCAR comprises a CD8 alpha hinge domain (SEQ ID NO:84). In some embodiments, the iCAR comprises a CD28 hinge domain (SEQ ID NO:85). In some embodiments, the iCAR comprises a PD-1 hinge domain (SEQ ID NO:86). In some embodiments, the iCAR comprises a LIR1 Ig3-4 hinge domain (SEQ ID NO:87). In some embodiments, the iCAR comprises a LIR1 Ig-4 hinge domain (SEQ ID NO:88). In some embodiments, the iCAR comprises a LIR1 52 aa hinge domain (SEQ ID NO:89). In some embodiments, the iCAR comprises a LIR1 36 aa hinge domain (SEQ ID NO:90). In some embodiments, the iCAR comprises a LIR1 30 aa hinge domain (SEQ ID NO:91). In some embodiments, the iCAR comprises a LIR1 8 aa hinge domain (SEQ ID NO:92). In some embodiments, the iCAR comprises a CD33 hinge domain (SEQ ID NO:93). In some embodiments, the iCAR comprises a KIR2DL1 hinge domain (SEQ ID NO:94). In some embodiments, the iCAR comprises a LIR1 26 aa hinge domain (SEQ ID NO: 289). In some embodiments, the iCAR comprises PD-1 (47) hinge domain (SEQ ID NO: 290). In some embodiments, the iCAR comprises PD-1 (42) hinge domain (SEQ ID NO: 291). In some embodiments, the iCAR comprises PD-1 (36) hinge domain (SEQ ID NO: 292). In some embodiments, the iCAR comprises PD-1 (30) hinge domain (SEQ ID NO: 293). In some embodiments, the iCAR comprises PD-1 (26) hinge domain (SEQ ID NO: 294). In some embodiments, the iCAR comprises PD-1 (20) hinge domain (SEQ ID NO: 295). In some embodiments, the iCAR comprises a CD8 alpha transmembrane domain (SEQ ID NO:95). In some embodiments, the iCAR comprises a CD28 transmembrane domain (SEQ ID NO:96). In some embodiments, the iCAR comprises a PD-1 transmembrane domain (SEQ ID NO:97). In some embodiments, the iCAR comprises a LIR1 transmembrane domain (SEQ ID NO:98). In some embodiments, the iCAR comprises a CD33 transmembrane domain (SEQ ID NO:99). In some embodiments, the iCAR comprises a KIR2DL1 transmembrane domain (SEQ ID NO:100). In some embodiments, the iCAR comprises a PD-1 inhibitory domain (SEQ ID NO:101). In some embodiments, the iCAR comprises a KIR2DL1 inhibitory domain (SEQ ID NO:102). In some embodiments, the iCAR comprises a KIR2DL2 inhibitory domain (SEQ ID NO:103). In some embodiments, the iCAR comprises a KIR2DL3 inhibitory domain (SEQ ID NO:104). In some embodiments, the iCAR comprises a KIR2DL4 inhibitory domain (SEQ ID NO:105). In some embodiments, the iCAR comprises a KIR2DL5A inhibitory domain (SEQ ID NO:106). In some embodiments, the iCAR comprises a KIR3DL1 inhibitory domain (SEQ ID NO:107). In some embodiments, the iCAR comprises a KIR3DL2 inhibitory domain (SEQ ID NO:108). In some embodiments, the iCAR comprises a KIR3DL3 inhibitory domain (SEQ ID NO:109). In some embodiments, the iCAR comprises a LAIR1 inhibitory domain (SEQ ID NO:110). In some embodiments, the iCAR comprises a CD22 inhibitory domain (SEQ ID NO:111). In some embodiments, the iCAR comprises a CD33 inhibitory domain (SEQ ID NO:112). In some embodiments, the iCAR comprises a SIGLEC5 inhibitory domain (SEQ ID NO:113). In some embodiments, the iCAR comprises a SIGLEC6 inhibitory domain (SEQ ID NO:114). In some embodiments, the iCAR comprises a SIGLEC7 inhibitory domain (SEQ ID NO:115). In some embodiments, the iCAR comprises a SIGLEC8 inhibitory domain (SEQ ID NO:116). In some embodiments, the iCAR comprises a SIGLEC9 inhibitory domain (SEQ ID NO:117). In some embodiments, the iCAR comprises a SIGLEC10 inhibitory domain (SEQ ID NO:118). In some embodiments, the iCAR comprises a SIGLEC11 inhibitory domain (SEQ ID NO:119). In some embodiments, the iCAR comprises a SIGLEC12 inhibitory domain (SEQ ID NO:120). In some embodiments, the iCAR comprises a PECAM1/CD31 inhibitory domain (SEQ ID NO:121). In some embodiments, the iCAR comprises a CD200R1 inhibitory domain (SEQ ID NO:122). In some embodiments, the iCAR comprises a FCRL1 inhibitory domain (SEQ ID NO:123). In some embodiments, the iCAR comprises a FCRL2 inhibitory domain (SEQ ID NO:124). In some embodiments, the iCAR comprises a FCRL3 inhibitory domain (SEQ ID NO:125). In some embodiments, the iCAR comprises a FCRL4 inhibitory domain (SEQ ID NO:126). In some embodiments, the iCAR comprises a FCRL5 inhibitory domain (SEQ ID NO:127). In some embodiments, the iCAR comprises a SLAMF1 inhibitory domain (SEQ ID NO:128). In some embodiments, the iCAR comprises a SLAMF5 inhibitory domain (SEQ ID NO:129). In some embodiments, the iCAR comprises a BTLA inhibitory domain (SEQ ID NO:130). In some embodiments, the iCAR comprises a LAG3 inhibitory domain (SEQ ID NO:131). In some embodiments, the iCAR comprises a 2B4 inhibitory domain (SEQ ID NO:132). In some embodiments, the iCAR comprises a CD160 inhibitory domain (SEQ ID NO:133). In some embodiments, the iCAR comprises a CEACAM1 inhibitory domain (SEQ ID NO:134). In some embodiments, the iCAR comprises a TIM3 inhibitory domain (SEQ ID NO:135). In some embodiments, the iCAR comprises a VISTA inhibitory domain (SEQ ID NO:136). In some embodiments, the iCAR comprises a TIGIT inhibitory domain (SEQ ID NO:137). In some embodiments, the iCAR comprises a SIRPalpha inhibitory domain (SEQ ID NO:138). In some embodiments, the iCAR comprises a FcγRIIB inhibitory domain (SEQ ID NO:139). In some embodiments, the iCAR comprises a CD5 inhibitory domain (SEQ ID NO:140). In some embodiments, the iCAR comprises a CD300a inhibitory domain (SEQ ID NO:141). In some embodiments, the iCAR comprises a CD300f inhibitory domain (SEQ ID NO:142). In some embodiments, the iCAR comprises a LIR1 inhibitory domain (SEQ ID NO:143). In some embodiments, the iCAR comprises a LIR2 inhibitory domain (SEQ ID NO:144). In some embodiments, the iCAR comprises a LIR3 inhibitory domain (SEQ ID NO:145). In some embodiments, the iCAR comprises a LIR5 inhibitory domain (SEQ ID NO:146). In some embodiments, the iCAR comprises a LIR8 inhibitory domain (SEQ ID NO:147). In some embodiments, the iCAR comprises a Ly9 inhibitory domain (SEQ ID NO:148). In some embodiments, the iCAR comprises a 2×PD1(G4S) inhibitory domain (SEQ ID NO:149). In some embodiments, the iCAR comprises a 2×PD1(PD1) inhibitory domain (SEQ ID NO:150). In some embodiments, the iCAR comprises a PVRIg inhibitory domain (SEQ ID NO:151). In some embodiments, the iCAR comprises an AA2AR inhibitory domain (SEQ ID NO:152). In some embodiments, the iCAR comprises a signal peptide upstream of the iCAR portion, wherein the signal peptide is a CD8 alpha signal peptide (SEQ ID NO: 161), a GM-CSF signal peptide (SEQ ID NO: 162), or a mIgK signal peptide (SEQ ID NO: 306).

In some embodiments, the iCAR comprises an scFv component comprising the VL and VH sequences of BBM.1 (SEQ ID NOs: 47 and 48). In some embodiments, the orientation of the iCAR VH and VL regions is VH-VL. In some embodiments, the orientation of the iCAR VH and VL regions is VL-VH. In some embodiments, the iCAR scFv comprises a (G4S)X3 linker (SEQ ID NO:81) linker that covalently connects the VH and the VL to form the iCAR scFv. In some embodiments, the iCAR scFv comprises a Whitlow linker (SEQ ID NO:82) linker that covalently connects the VH and the VL to form the iCAR scFv. In some embodiments, the iCAR comprises a CD8 alpha hinge domain (SEQ ID NO:84). In some embodiments, the iCAR comprises a CD28 hinge domain (SEQ ID NO:85). In some embodiments, the iCAR comprises a PD-1 hinge domain (SEQ ID NO:86). In some embodiments, the iCAR comprises a LIR1 Ig3-4 hinge domain (SEQ ID NO:87). In some embodiments, the iCAR comprises a LIR1 Ig-4 hinge domain (SEQ ID NO:88). In some embodiments, the iCAR comprises a LIR1 52 aa hinge domain (SEQ ID NO:89). In some embodiments, the iCAR comprises a LIR1 36 aa hinge domain (SEQ ID NO:90). In some embodiments, the iCAR comprises a LIR1 30 aa hinge domain (SEQ ID NO:91). In some embodiments, the iCAR comprises a LIR1 8 aa hinge domain (SEQ ID NO:92). In some embodiments, the iCAR comprises a CD33 hinge domain (SEQ ID NO:93). In some embodiments, the iCAR comprises a KIR2DL1 hinge domain (SEQ ID NO:94). In some embodiments, the iCAR comprises a LIR1 26 aa hinge domain (SEQ ID NO: 289). In some embodiments, the iCAR comprises PD-1 (47) hinge domain (SEQ ID NO: 290). In some embodiments, the iCAR comprises PD-1 (42) hinge domain (SEQ ID NO: 291). In some embodiments, the iCAR comprises PD-1 (36) hinge domain (SEQ ID NO: 292). In some embodiments, the iCAR comprises PD-1 (30) hinge domain (SEQ ID NO: 293). In some embodiments, the iCAR comprises PD-1 (26) hinge domain (SEQ ID NO: 294). In some embodiments, the iCAR comprises PD-1 (20) hinge domain (SEQ ID NO: 295). In some embodiments, the iCAR comprises a CD8 alpha transmembrane domain (SEQ ID NO:95). In some embodiments, the iCAR comprises a CD28 transmembrane domain (SEQ ID NO:96). In some embodiments, the iCAR comprises a PD-1 transmembrane domain (SEQ ID NO:97). In some embodiments, the iCAR comprises a LIR1 transmembrane domain (SEQ ID NO:98). In some embodiments, the iCAR comprises a CD33 transmembrane domain (SEQ ID NO:99). In some embodiments, the iCAR comprises a KIR2DL1 transmembrane domain (SEQ ID NO:100). In some embodiments, the iCAR comprises a PD-1 inhibitory domain (SEQ ID NO:101). In some embodiments, the iCAR comprises a KIR2DL1 inhibitory domain (SEQ ID NO:102). In some embodiments, the iCAR comprises a KIR2DL2 inhibitory domain (SEQ ID NO:103). In some embodiments, the iCAR comprises a KIR2DL3 inhibitory domain (SEQ ID NO:104). In some embodiments, the iCAR comprises a KIR2DL4 inhibitory domain (SEQ ID NO:105). In some embodiments, the iCAR comprises a KIR2DL5A inhibitory domain (SEQ ID NO:106). In some embodiments, the iCAR comprises a KIR3DL1 inhibitory domain (SEQ ID NO:107). In some embodiments, the iCAR comprises a KIR3DL2 inhibitory domain (SEQ ID NO:108). In some embodiments, the iCAR comprises a KIR3DL3 inhibitory domain (SEQ ID NO:109). In some embodiments, the iCAR comprises a LAIR1 inhibitory domain (SEQ ID NO:110). In some embodiments, the iCAR comprises a CD22 inhibitory domain (SEQ ID NO:111). In some embodiments, the iCAR comprises a CD33 inhibitory domain (SEQ ID NO:112). In some embodiments, the iCAR comprises a SIGLEC5 inhibitory domain (SEQ ID NO:113). In some embodiments, the iCAR comprises a SIGLEC6 inhibitory domain (SEQ ID NO:114). In some embodiments, the iCAR comprises a SIGLEC7 inhibitory domain (SEQ ID NO:115). In some embodiments, the iCAR comprises a SIGLEC8 inhibitory domain (SEQ ID NO:116). In some embodiments, the iCAR comprises a SIGLEC9 inhibitory domain (SEQ ID NO:117). In some embodiments, the iCAR comprises a SIGLEC10 inhibitory domain (SEQ ID NO:118). In some embodiments, the iCAR comprises a SIGLEC11 inhibitory domain (SEQ ID NO:119). In some embodiments, the iCAR comprises a SIGLEC12 inhibitory domain (SEQ ID NO:120). In some embodiments, the iCAR comprises a PECAM1/CD31 inhibitory domain (SEQ ID NO:121). In some embodiments, the iCAR comprises a CD200R1 inhibitory domain (SEQ ID NO:122). In some embodiments, the iCAR comprises a FCRL1 inhibitory domain (SEQ ID NO:123). In some embodiments, the iCAR comprises a FCRL2 inhibitory domain (SEQ ID NO:124). In some embodiments, the iCAR comprises a FCRL3 inhibitory domain (SEQ ID NO:125). In some embodiments, the iCAR comprises a FCRL4 inhibitory domain (SEQ ID NO:126). In some embodiments, the iCAR comprises a FCRL5 inhibitory domain (SEQ ID NO:127). In some embodiments, the iCAR comprises a SLAMF1 inhibitory domain (SEQ ID NO:128). In some embodiments, the iCAR comprises a SLAMF5 inhibitory domain (SEQ ID NO:129). In some embodiments, the iCAR comprises a BTLA inhibitory domain (SEQ ID NO:130). In some embodiments, the iCAR comprises a LAG3 inhibitory domain (SEQ ID NO:131). In some embodiments, the iCAR comprises a 2B4 inhibitory domain (SEQ ID NO:132). In some embodiments, the iCAR comprises a CD160 inhibitory domain (SEQ ID NO:133). In some embodiments, the iCAR comprises a CEACAM1 inhibitory domain (SEQ ID NO:134). In some embodiments, the iCAR comprises a TIM3 inhibitory domain (SEQ ID NO:135). In some embodiments, the iCAR comprises a VISTA inhibitory domain (SEQ ID NO:136). In some embodiments, the iCAR comprises a TIGIT inhibitory domain (SEQ ID NO:137). In some embodiments, the iCAR comprises a SIRPalpha inhibitory domain (SEQ ID NO:138). In some embodiments, the iCAR comprises a FcγRIIB inhibitory domain (SEQ ID NO:139). In some embodiments, the iCAR comprises a CD5 inhibitory domain (SEQ ID NO:140). In some embodiments, the iCAR comprises a CD300a inhibitory domain (SEQ ID NO:141). In some embodiments, the iCAR comprises a CD300f inhibitory domain (SEQ ID NO:142). In some embodiments, the iCAR comprises a LIR1 inhibitory domain (SEQ ID NO:143). In some embodiments, the iCAR comprises a LIR2 inhibitory domain (SEQ ID NO:144). In some embodiments, the iCAR comprises a LIR3 inhibitory domain (SEQ ID NO:145). In some embodiments, the iCAR comprises a LIR5 inhibitory domain (SEQ ID NO:146). In some embodiments, the iCAR comprises a LIR8 inhibitory domain (SEQ ID NO:147). In some embodiments, the iCAR comprises a Ly9 inhibitory domain (SEQ ID NO:148). In some embodiments, the iCAR comprises a 2×PD1(G4S) inhibitory domain (SEQ ID NO:149). In some embodiments, the iCAR comprises a 2×PD1(PD1) inhibitory domain (SEQ ID NO:150). In some embodiments, the iCAR comprises a PVRIg inhibitory domain (SEQ ID NO:151). In some embodiments, the iCAR comprises an AA2AR inhibitory domain (SEQ ID NO:152). In some embodiments, the iCAR comprises a signal peptide upstream of the iCAR portion, wherein the signal peptide is a CD8 alpha signal peptide (SEQ ID NO: 161), a GM-CSF signal peptide (SEQ ID NO: 162), or a mIgK signal peptide (SEQ ID NO: 306).

In some embodiments, the iCAR comprises an scFv component comprising the VL and VH sequences of SN66E3.1 (SEQ ID NOs: 49 and 50). In some embodiments, the orientation of the iCAR VH and VL regions is VH-VL. In some embodiments, the orientation of the iCAR VH and VL regions is VL-VH. In some embodiments, the iCAR scFv comprises a (G4S)X3 linker (SEQ ID NO:81) linker that covalently connects the VH and the VL to form the iCAR scFv. In some embodiments, the iCAR scFv comprises a Whitlow linker (SEQ ID NO:82) linker that covalently connects the VH and the VL to form the iCAR scFv. In some embodiments, the iCAR comprises a CD8 alpha hinge domain (SEQ ID NO:84). In some embodiments, the iCAR comprises a CD28 hinge domain (SEQ ID NO:85). In some embodiments, the iCAR comprises a PD-1 hinge domain (SEQ ID NO:86). In some embodiments, the iCAR comprises a LIR1 Ig3-4 hinge domain (SEQ ID NO:87). In some embodiments, the iCAR comprises a LIR1 Ig-4 hinge domain (SEQ ID NO:88). In some embodiments, the iCAR comprises a LIR1 52 aa hinge domain (SEQ ID NO:89). In some embodiments, the iCAR comprises a LIR1 36 aa hinge domain (SEQ ID NO:90). In some embodiments, the iCAR comprises a LIR1 30 aa hinge domain (SEQ ID NO:91). In some embodiments, the iCAR comprises a LIR1 8 aa hinge domain (SEQ ID NO:92). In some embodiments, the iCAR comprises a CD33 hinge domain (SEQ ID NO:93). In some embodiments, the iCAR comprises a KIR2DL1 hinge domain (SEQ ID NO:94). In some embodiments, the iCAR comprises a LIR1 26 aa hinge domain (SEQ ID NO: 289). In some embodiments, the iCAR comprises PD-1 (47) hinge domain (SEQ ID NO: 290). In some embodiments, the iCAR comprises PD-1 (42) hinge domain (SEQ ID NO: 291). In some embodiments, the iCAR comprises PD-1 (36) hinge domain (SEQ ID NO: 292). In some embodiments, the iCAR comprises PD-1 (30) hinge domain (SEQ ID NO: 293). In some embodiments, the iCAR comprises PD-1 (26) hinge domain (SEQ ID NO: 294). In some embodiments, the iCAR comprises PD-1 (20) hinge domain (SEQ ID NO: 295). In some embodiments, the iCAR comprises a CD8 alpha transmembrane domain (SEQ ID NO:95). In some embodiments, the iCAR comprises a CD28 transmembrane domain (SEQ ID NO:96). In some embodiments, the iCAR comprises a PD-1 transmembrane domain (SEQ ID NO:97). In some embodiments, the iCAR comprises a LIR1 transmembrane domain (SEQ ID NO:98). In some embodiments, the iCAR comprises a CD33 transmembrane domain (SEQ ID NO:99). In some embodiments, the iCAR comprises a KIR2DL1 transmembrane domain (SEQ ID NO:100). In some embodiments, the iCAR comprises a PD-1 inhibitory domain (SEQ ID NO:101). In some embodiments, the iCAR comprises a KIR2DL1 inhibitory domain (SEQ ID NO:102). In some embodiments, the iCAR comprises a KIR2DL2 inhibitory domain (SEQ ID NO:103). In some embodiments, the iCAR comprises a KIR2DL3 inhibitory domain (SEQ ID NO:104). In some embodiments, the iCAR comprises a KIR2DL4 inhibitory domain (SEQ ID NO:105). In some embodiments, the iCAR comprises a KIR2DL5A inhibitory domain (SEQ ID NO:106). In some embodiments, the iCAR comprises a KIR3DL1 inhibitory domain (SEQ ID NO:107). In some embodiments, the iCAR comprises a KIR3DL2 inhibitory domain (SEQ ID NO:108). In some embodiments, the iCAR comprises a KIR3DL3 inhibitory domain (SEQ ID NO:109). In some embodiments, the iCAR comprises a LAIR1 inhibitory domain (SEQ ID NO:110). In some embodiments, the iCAR comprises a CD22 inhibitory domain (SEQ ID NO:111). In some embodiments, the iCAR comprises a CD33 inhibitory domain (SEQ ID NO:112). In some embodiments, the iCAR comprises a SIGLEC5 inhibitory domain (SEQ ID NO:113). In some embodiments, the iCAR comprises a SIGLEC6 inhibitory domain (SEQ ID NO:114). In some embodiments, the iCAR comprises a SIGLEC7 inhibitory domain (SEQ ID NO:115). In some embodiments, the iCAR comprises a SIGLEC8 inhibitory domain (SEQ ID NO:116). In some embodiments, the iCAR comprises a SIGLEC9 inhibitory domain (SEQ ID NO:117). In some embodiments, the iCAR comprises a SIGLEC10 inhibitory domain (SEQ ID NO:118). In some embodiments, the iCAR comprises a SIGLEC11 inhibitory domain (SEQ ID NO:119). In some embodiments, the iCAR comprises a SIGLEC12 inhibitory domain (SEQ ID NO:120). In some embodiments, the iCAR comprises a PECAM1/CD31 inhibitory domain (SEQ ID NO:121). In some embodiments, the iCAR comprises a CD200R1 inhibitory domain (SEQ ID NO:122). In some embodiments, the iCAR comprises a FCRL1 inhibitory domain (SEQ ID NO:123). In some embodiments, the iCAR comprises a FCRL2 inhibitory domain (SEQ ID NO:124). In some embodiments, the iCAR comprises a FCRL3 inhibitory domain (SEQ ID NO:125). In some embodiments, the iCAR comprises a FCRL4 inhibitory domain (SEQ ID NO:126). In some embodiments, the iCAR comprises a FCRL5 inhibitory domain (SEQ ID NO:127). In some embodiments, the iCAR comprises a SLAMF1 inhibitory domain (SEQ ID NO:128). In some embodiments, the iCAR comprises a SLAMF5 inhibitory domain (SEQ ID NO:129). In some embodiments, the iCAR comprises a BTLA inhibitory domain (SEQ ID NO:130). In some embodiments, the iCAR comprises a LAG3 inhibitory domain (SEQ ID NO:131). In some embodiments, the iCAR comprises a 2B4 inhibitory domain (SEQ ID NO:132). In some embodiments, the iCAR comprises a CD160 inhibitory domain (SEQ ID NO:133). In some embodiments, the iCAR comprises a CEACAM1 inhibitory domain (SEQ ID NO:134). In some embodiments, the iCAR comprises a TIM3 inhibitory domain (SEQ ID NO:135). In some embodiments, the iCAR comprises a VISTA inhibitory domain (SEQ ID NO:136). In some embodiments, the iCAR comprises a TIGIT inhibitory domain (SEQ ID NO:137). In some embodiments, the iCAR comprises a SIRPalpha inhibitory domain (SEQ ID NO:138). In some embodiments, the iCAR comprises a FcγRIIB inhibitory domain (SEQ ID NO:139). In some embodiments, the iCAR comprises a CD5 inhibitory domain (SEQ ID NO:140). In some embodiments, the iCAR comprises a CD300a inhibitory domain (SEQ ID NO:141). In some embodiments, the iCAR comprises a CD300f inhibitory domain (SEQ ID NO:142). In some embodiments, the iCAR comprises a LIR1 inhibitory domain (SEQ ID NO:143). In some embodiments, the iCAR comprises a LIR2 inhibitory domain (SEQ ID NO:144). In some embodiments, the iCAR comprises a LIR3 inhibitory domain (SEQ ID NO:145). In some embodiments, the iCAR comprises a LIR5 inhibitory domain (SEQ ID NO:146). In some embodiments, the iCAR comprises a LIR8 inhibitory domain (SEQ ID NO:147). In some embodiments, the iCAR comprises a Ly9 inhibitory domain (SEQ ID NO:148). In some embodiments, the iCAR comprises a 2×PD1(G4S) inhibitory domain (SEQ ID NO:149). In some embodiments, the iCAR comprises a 2×PD1(PD1) inhibitory domain (SEQ ID NO:150). In some embodiments, the iCAR comprises a PVRIg inhibitory domain (SEQ ID NO:151). In some embodiments, the iCAR comprises an AA2AR inhibitory domain (SEQ ID NO:152). In some embodiments, the iCAR comprises a signal peptide upstream of the iCAR portion, wherein the signal peptide is a CD8 alpha signal peptide (SEQ ID NO: 161), a GM-CSF signal peptide (SEQ ID NO: 162), or a mIgK signal peptide (SEQ ID NO: 306).

In some embodiments, the iCAR comprises an scFv component comprising the VL and VH sequences of Ha5C2.A2 (SEQ ID NOs: 51 and 52). In some embodiments, the orientation of the iCAR VH and VL regions is VH-VL. In some embodiments, the orientation of the iCAR VH and VL regions is VL-VH. In some embodiments, the iCAR scFv comprises a (G4S)X3 linker (SEQ ID NO:81) linker that covalently connects the VH and the VL to form the iCAR scFv. In some embodiments, the iCAR scFv comprises a Whitlow linker (SEQ ID NO:82) linker that covalently connects the VH and the VL to form the iCAR scFv. In some embodiments, the iCAR comprises a CD8 alpha hinge domain (SEQ ID NO:84). In some embodiments, the iCAR comprises a CD28 hinge domain (SEQ ID NO:85). In some embodiments, the iCAR comprises a PD-1 hinge domain (SEQ ID NO:86). In some embodiments, the iCAR comprises a LIR1 Ig3-4 hinge domain (SEQ ID NO:87). In some embodiments, the iCAR comprises a LIR1 Ig-4 hinge domain (SEQ ID NO:88). In some embodiments, the iCAR comprises a LIR1 52 aa hinge domain (SEQ ID NO:89). In some embodiments, the iCAR comprises a LIR1 36 aa hinge domain (SEQ ID NO:90). In some embodiments, the iCAR comprises a LIR1 30 aa hinge domain (SEQ ID NO:91). In some embodiments, the iCAR comprises a LIR1 8 aa hinge domain (SEQ ID NO:92). In some embodiments, the iCAR comprises a CD33 hinge domain (SEQ ID NO:93). In some embodiments, the iCAR comprises a KIR2DL1 hinge domain (SEQ ID NO:94). In some embodiments, the iCAR comprises a LIR1 26 aa hinge domain (SEQ ID NO: 289). In some embodiments, the iCAR comprises PD-1 (47) hinge domain (SEQ ID NO: 290). In some embodiments, the iCAR comprises PD-1 (42) hinge domain (SEQ ID NO: 291). In some embodiments, the iCAR comprises PD-1 (36) hinge domain (SEQ ID NO: 292). In some embodiments, the iCAR comprises PD-1 (30) hinge domain (SEQ ID NO: 293). In some embodiments, the iCAR comprises PD-1 (26) hinge domain (SEQ ID NO: 294). In some embodiments, the iCAR comprises PD-1 (20) hinge domain (SEQ ID NO: 295). In some embodiments, the iCAR comprises a CD8 alpha transmembrane domain (SEQ ID NO:95). In some embodiments, the iCAR comprises a CD28 transmembrane domain (SEQ ID NO:96). In some embodiments, the iCAR comprises a PD-1 transmembrane domain (SEQ ID NO:97). In some embodiments, the iCAR comprises a LIR1 transmembrane domain (SEQ ID NO:98). In some embodiments, the iCAR comprises a CD33 transmembrane domain (SEQ ID NO:99). In some embodiments, the iCAR comprises a KIR2DL1 transmembrane domain (SEQ ID NO:100). In some embodiments, the iCAR comprises a PD-1 inhibitory domain (SEQ ID NO:101). In some embodiments, the iCAR comprises a KIR2DL1 inhibitory domain (SEQ ID NO:102). In some embodiments, the iCAR comprises a KIR2DL2 inhibitory domain (SEQ ID NO:103). In some embodiments, the iCAR comprises a KIR2DL3 inhibitory domain (SEQ ID NO:104). In some embodiments, the iCAR comprises a KIR2DL4 inhibitory domain (SEQ ID NO:105). In some embodiments, the iCAR comprises a KIR2DL5A inhibitory domain (SEQ ID NO:106). In some embodiments, the iCAR comprises a KIR3DL1 inhibitory domain (SEQ ID NO:107). In some embodiments, the iCAR comprises a KIR3DL2 inhibitory domain (SEQ ID NO:108). In some embodiments, the iCAR comprises a KIR3DL3 inhibitory domain (SEQ ID NO:109). In some embodiments, the iCAR comprises a LAIR1 inhibitory domain (SEQ ID NO:110). In some embodiments, the iCAR comprises a CD22 inhibitory domain (SEQ ID NO:111). In some embodiments, the iCAR comprises a CD33 inhibitory domain (SEQ ID NO:112). In some embodiments, the iCAR comprises a SIGLEC5 inhibitory domain (SEQ ID NO:113). In some embodiments, the iCAR comprises a SIGLEC6 inhibitory domain (SEQ ID NO:114). In some embodiments, the iCAR comprises a SIGLEC7 inhibitory domain (SEQ ID NO:115). In some embodiments, the iCAR comprises a SIGLEC8 inhibitory domain (SEQ ID NO:116). In some embodiments, the iCAR comprises a SIGLEC9 inhibitory domain (SEQ ID NO:117). In some embodiments, the iCAR comprises a SIGLEC10 inhibitory domain (SEQ ID NO:118). In some embodiments, the iCAR comprises a SIGLEC11 inhibitory domain (SEQ ID NO:119). In some embodiments, the iCAR comprises a SIGLEC12 inhibitory domain (SEQ ID NO:120). In some embodiments, the iCAR comprises a PECAM1/CD31 inhibitory domain (SEQ ID NO:121). In some embodiments, the iCAR comprises a CD200R1 inhibitory domain (SEQ ID NO:122). In some embodiments, the iCAR comprises a FCRL1 inhibitory domain (SEQ ID NO:123). In some embodiments, the iCAR comprises a FCRL2 inhibitory domain (SEQ ID NO:124). In some embodiments, the iCAR comprises a FCRL3 inhibitory domain (SEQ ID NO:125). In some embodiments, the iCAR comprises a FCRL4 inhibitory domain (SEQ ID NO:126). In some embodiments, the iCAR comprises a FCRL5 inhibitory domain (SEQ ID NO:127). In some embodiments, the iCAR comprises a SLAMF1 inhibitory domain (SEQ ID NO:128). In some embodiments, the iCAR comprises a SLAMF5 inhibitory domain (SEQ ID NO:129). In some embodiments, the iCAR comprises a BTLA inhibitory domain (SEQ ID NO:130). In some embodiments, the iCAR comprises a LAG3 inhibitory domain (SEQ ID NO:131). In some embodiments, the iCAR comprises a 2B4 inhibitory domain (SEQ ID NO:132). In some embodiments, the iCAR comprises a CD160 inhibitory domain (SEQ ID NO:133). In some embodiments, the iCAR comprises a CEACAM1 inhibitory domain (SEQ ID NO:134). In some embodiments, the iCAR comprises a TIM3 inhibitory domain (SEQ ID NO:135). In some embodiments, the iCAR comprises a VISTA inhibitory domain (SEQ ID NO:136). In some embodiments, the iCAR comprises a TIGIT inhibitory domain (SEQ ID NO:137). In some embodiments, the iCAR comprises a SIRPalpha inhibitory domain (SEQ ID NO:138). In some embodiments, the iCAR comprises a FcγRIIB inhibitory domain (SEQ ID NO:139). In some embodiments, the iCAR comprises a CD5 inhibitory domain (SEQ ID NO:140). In some embodiments, the iCAR comprises a CD300a inhibitory domain (SEQ ID NO:141). In some embodiments, the iCAR comprises a CD300f inhibitory domain (SEQ ID NO:142). In some embodiments, the iCAR comprises a LIR1 inhibitory domain (SEQ ID NO:143). In some embodiments, the iCAR comprises a LIR2 inhibitory domain (SEQ ID NO:144). In some embodiments, the iCAR comprises a LIR3 inhibitory domain (SEQ ID NO:145). In some embodiments, the iCAR comprises a LIR5 inhibitory domain (SEQ ID NO:146). In some embodiments, the iCAR comprises a LIR8 inhibitory domain (SEQ ID NO:147). In some embodiments, the iCAR comprises a Ly9 inhibitory domain (SEQ ID NO:148). In some embodiments, the iCAR comprises a 2×PD1(G4S) inhibitory domain (SEQ ID NO:149). In some embodiments, the iCAR comprises a 2×PD1(PD1) inhibitory domain (SEQ ID NO:150). In some embodiments, the iCAR comprises a PVRIg inhibitory domain (SEQ ID NO:151). In some embodiments, the iCAR comprises an AA2AR inhibitory domain (SEQ ID NO:152). In some embodiments, the iCAR comprises a signal peptide upstream of the iCAR portion, wherein the signal peptide is a CD8 alpha signal peptide (SEQ ID NO: 161), a GM-CSF signal peptide (SEQ ID NO: 162), or a mIgK signal peptide (SEQ ID NO: 306).

In some embodiments, the iCAR comprises an scFv component comprising the VL and VH sequences of MWB1 (SEQ ID NOs: 53 and 54). In some embodiments, the orientation of the iCAR VH and VL regions is VH-VL. In some embodiments, the orientation of the iCAR VH and VL regions is VL-VH. In some embodiments, the iCAR scFv comprises a (G4S)X3 linker (SEQ ID NO:81) linker that covalently connects the VH and the VL to form the iCAR scFv. In some embodiments, the iCAR scFv comprises a Whitlow linker (SEQ ID NO:82) linker that covalently connects the VH and the VL to form the iCAR scFv. In some embodiments, the iCAR comprises a CD8 alpha hinge domain (SEQ ID NO:84). In some embodiments, the iCAR comprises a CD28 hinge domain (SEQ ID NO:85). In some embodiments, the iCAR comprises a PD-1 hinge domain (SEQ ID NO:86). In some embodiments, the iCAR comprises a LIR1 Ig3-4 hinge domain (SEQ ID NO:87). In some embodiments, the iCAR comprises a LIR1 Ig-4 hinge domain (SEQ ID NO:88). In some embodiments, the iCAR comprises a LIR1 52 aa hinge domain (SEQ ID NO:89). In some embodiments, the iCAR comprises a LIR1 36 aa hinge domain (SEQ ID NO:90). In some embodiments, the iCAR comprises a LIR1 30 aa hinge domain (SEQ ID NO:91). In some embodiments, the iCAR comprises a LIR1 8 aa hinge domain (SEQ ID NO:92). In some embodiments, the iCAR comprises a CD33 hinge domain (SEQ ID NO:93). In some embodiments, the iCAR comprises a KIR2DL1 hinge domain (SEQ ID NO:94). In some embodiments, the iCAR comprises a LIR1 26 aa hinge domain (SEQ ID NO: 289). In some embodiments, the iCAR comprises PD-1 (47) hinge domain (SEQ ID NO: 290). In some embodiments, the iCAR comprises PD-1 (42) hinge domain (SEQ ID NO: 291). In some embodiments, the iCAR comprises PD-1 (36) hinge domain (SEQ ID NO: 292). In some embodiments, the iCAR comprises PD-1 (30) hinge domain (SEQ ID NO: 293). In some embodiments, the iCAR comprises PD-1 (26) hinge domain (SEQ ID NO: 294). In some embodiments, the iCAR comprises PD-1 (20) hinge domain (SEQ ID NO: 295). In some embodiments, the iCAR comprises a CD8 alpha transmembrane domain (SEQ ID NO:95). In some embodiments, the iCAR comprises a CD28 transmembrane domain (SEQ ID NO:96). In some embodiments, the iCAR comprises a PD-1 transmembrane domain (SEQ ID NO:97). In some embodiments, the iCAR comprises a LIR1 transmembrane domain (SEQ ID NO:98). In some embodiments, the iCAR comprises a CD33 transmembrane domain (SEQ ID NO:99). In some embodiments, the iCAR comprises a KIR2DL1 transmembrane domain (SEQ ID NO:100). In some embodiments, the iCAR comprises a PD-1 inhibitory domain (SEQ ID NO:101). In some embodiments, the iCAR comprises a KIR2DL1 inhibitory domain (SEQ ID NO:102). In some embodiments, the iCAR comprises a KIR2DL2 inhibitory domain (SEQ ID NO:103). In some embodiments, the iCAR comprises a KIR2DL3 inhibitory domain (SEQ ID NO:104). In some embodiments, the iCAR comprises a KIR2DL4 inhibitory domain (SEQ ID NO:105). In some embodiments, the iCAR comprises a KIR2DL5A inhibitory domain (SEQ ID NO:106). In some embodiments, the iCAR comprises a KIR3DL1 inhibitory domain (SEQ ID NO:107). In some embodiments, the iCAR comprises a KIR3DL2 inhibitory domain (SEQ ID NO:108). In some embodiments, the iCAR comprises a KIR3DL3 inhibitory domain (SEQ ID NO:109). In some embodiments, the iCAR comprises a LAIR1 inhibitory domain (SEQ ID NO:110). In some embodiments, the iCAR comprises a CD22 inhibitory domain (SEQ ID NO:111). In some embodiments, the iCAR comprises a CD33 inhibitory domain (SEQ ID NO:112). In some embodiments, the iCAR comprises a SIGLEC5 inhibitory domain (SEQ ID NO:113). In some embodiments, the iCAR comprises a SIGLEC6 inhibitory domain (SEQ ID NO:114). In some embodiments, the iCAR comprises a SIGLEC7 inhibitory domain (SEQ ID NO:115). In some embodiments, the iCAR comprises a SIGLEC8 inhibitory domain (SEQ ID NO:116). In some embodiments, the iCAR comprises a SIGLEC9 inhibitory domain (SEQ ID NO:117). In some embodiments, the iCAR comprises a SIGLEC10 inhibitory domain (SEQ ID NO:118). In some embodiments, the iCAR comprises a SIGLEC11 inhibitory domain (SEQ ID NO:119). In some embodiments, the iCAR comprises a SIGLEC12 inhibitory domain (SEQ ID NO:120). In some embodiments, the iCAR comprises a PECAM1/CD31 inhibitory domain (SEQ ID NO:121). In some embodiments, the iCAR comprises a CD200R1 inhibitory domain (SEQ ID NO:122). In some embodiments, the iCAR comprises a FCRL1 inhibitory domain (SEQ ID NO:123). In some embodiments, the iCAR comprises a FCRL2 inhibitory domain (SEQ ID NO:124). In some embodiments, the iCAR comprises a FCRL3 inhibitory domain (SEQ ID NO:125). In some embodiments, the iCAR comprises a FCRL4 inhibitory domain (SEQ ID NO:126). In some embodiments, the iCAR comprises a FCRL5 inhibitory domain (SEQ ID NO:127). In some embodiments, the iCAR comprises a SLAMF1 inhibitory domain (SEQ ID NO:128). In some embodiments, the iCAR comprises a SLAMF5 inhibitory domain (SEQ ID NO:129). In some embodiments, the iCAR comprises a BTLA inhibitory domain (SEQ ID NO:130). In some embodiments, the iCAR comprises a LAG3 inhibitory domain (SEQ ID NO:131). In some embodiments, the iCAR comprises a 2B4 inhibitory domain (SEQ ID NO:132). In some embodiments, the iCAR comprises a CD160 inhibitory domain (SEQ ID NO:133). In some embodiments, the iCAR comprises a CEACAM1 inhibitory domain (SEQ ID NO:134). In some embodiments, the iCAR comprises a TIM3 inhibitory domain (SEQ ID NO:135). In some embodiments, the iCAR comprises a VISTA inhibitory domain (SEQ ID NO:136). In some embodiments, the iCAR comprises a TIGIT inhibitory domain (SEQ ID NO:137). In some embodiments, the iCAR comprises a SIRPalpha inhibitory domain (SEQ ID NO:138). In some embodiments, the iCAR comprises a FcγRIIB inhibitory domain (SEQ ID NO:139). In some embodiments, the iCAR comprises a CD5 inhibitory domain (SEQ ID NO:140). In some embodiments, the iCAR comprises a CD300a inhibitory domain (SEQ ID NO:141). In some embodiments, the iCAR comprises a CD300f inhibitory domain (SEQ ID NO:142). In some embodiments, the iCAR comprises a LIR1 inhibitory domain (SEQ ID NO:143). In some embodiments, the iCAR comprises a LIR2 inhibitory domain (SEQ ID NO:144). In some embodiments, the iCAR comprises a LIR3 inhibitory domain (SEQ ID NO:145). In some embodiments, the iCAR comprises a LIR5 inhibitory domain (SEQ ID NO:146). In some embodiments, the iCAR comprises a LIR8 inhibitory domain (SEQ ID NO:147). In some embodiments, the iCAR comprises a Ly9 inhibitory domain (SEQ ID NO:148). In some embodiments, the iCAR comprises a 2×PD1(G4S) inhibitory domain (SEQ ID NO:149). In some embodiments, the iCAR comprises a 2×PD1(PD1) inhibitory domain (SEQ ID NO:150). In some embodiments, the iCAR comprises a PVRIg inhibitory domain (SEQ ID NO:151). In some embodiments, the iCAR comprises an AA2AR inhibitory domain (SEQ ID NO:152). In some embodiments, the iCAR comprises a signal peptide upstream of the iCAR portion, wherein the signal peptide is a CD8 alpha signal peptide (SEQ ID NO: 161), a GM-CSF signal peptide (SEQ ID NO: 162), or a mIgK signal peptide (SEQ ID NO: 306).

In some embodiments, the iCAR comprises an scFv component comprising the VL and VH sequences of MWB1-mod (MWB1.1) (SEQ ID NOs: 55 and 56). In some embodiments, the orientation of the iCAR VH and VL regions is VH-VL. In some embodiments, the orientation of the iCAR VH and VL regions is VL-VH. In some embodiments, the iCAR scFv comprises a (G4S)X3 linker (SEQ ID NO:81) linker that covalently connects the VH and the VL to form the iCAR scFv. In some embodiments, the iCAR scFv comprises a Whitlow linker (SEQ ID NO:82) linker that covalently connects the VH and the VL to form the iCAR scFv. In some embodiments, the iCAR comprises a CD8 alpha hinge domain (SEQ ID NO:84). In some embodiments, the iCAR comprises a CD28 hinge domain (SEQ ID NO:85). In some embodiments, the iCAR comprises a PD-1 hinge domain (SEQ ID NO:86). In some embodiments, the iCAR comprises a LIR1 Ig3-4 hinge domain (SEQ ID NO:87). In some embodiments, the iCAR comprises a LIR1 Ig-4 hinge domain (SEQ ID NO:88). In some embodiments, the iCAR comprises a LIR1 52 aa hinge domain (SEQ ID NO:89). In some embodiments, the iCAR comprises a LIR1 36 aa hinge domain (SEQ ID NO:90). In some embodiments, the iCAR comprises a LIR1 30 aa hinge domain (SEQ ID NO:91). In some embodiments, the iCAR comprises a LIR1 8 aa hinge domain (SEQ ID NO:92). In some embodiments, the iCAR comprises a CD33 hinge domain (SEQ ID NO:93). In some embodiments, the iCAR comprises a KIR2DL1 hinge domain (SEQ ID NO:94). In some embodiments, the iCAR comprises a LIR1 26 aa hinge domain (SEQ ID NO: 289). In some embodiments, the iCAR comprises PD-1 (47) hinge domain (SEQ ID NO: 290). In some embodiments, the iCAR comprises PD-1 (42) hinge domain (SEQ ID NO: 291). In some embodiments, the iCAR comprises PD-1 (36) hinge domain (SEQ ID NO: 292). In some embodiments, the iCAR comprises PD-1 (30) hinge domain (SEQ ID NO: 293). In some embodiments, the iCAR comprises PD-1 (26) hinge domain (SEQ ID NO: 294). In some embodiments, the iCAR comprises PD-1 (20) hinge domain (SEQ ID NO: 295). In some embodiments, the iCAR comprises a CD8 alpha transmembrane domain (SEQ ID NO:95). In some embodiments, the iCAR comprises a CD28 transmembrane domain (SEQ ID NO:96). In some embodiments, the iCAR comprises a PD-1 transmembrane domain (SEQ ID NO:97). In some embodiments, the iCAR comprises a LIR1 transmembrane domain (SEQ ID NO:98). In some embodiments, the iCAR comprises a CD33 transmembrane domain (SEQ ID NO:99). In some embodiments, the iCAR comprises a KIR2DL1 transmembrane domain (SEQ ID NO:100). In some embodiments, the iCAR comprises a PD-1 inhibitory domain (SEQ ID NO:101). In some embodiments, the iCAR comprises a KIR2DL1 inhibitory domain (SEQ ID NO:102). In some embodiments, the iCAR comprises a KIR2DL2 inhibitory domain (SEQ ID NO:103). In some embodiments, the iCAR comprises a KIR2DL3 inhibitory domain (SEQ ID NO:104). In some embodiments, the iCAR comprises a KIR2DL4 inhibitory domain (SEQ ID NO:105). In some embodiments, the iCAR comprises a KIR2DL5A inhibitory domain (SEQ ID NO:106). In some embodiments, the iCAR comprises a KIR3DL1 inhibitory domain (SEQ ID NO:107). In some embodiments, the iCAR comprises a KIR3DL2 inhibitory domain (SEQ ID NO:108). In some embodiments, the iCAR comprises a KIR3DL3 inhibitory domain (SEQ ID NO:109). In some embodiments, the iCAR comprises a LAIR1 inhibitory domain (SEQ ID NO:110). In some embodiments, the iCAR comprises a CD22 inhibitory domain (SEQ ID NO:111). In some embodiments, the iCAR comprises a CD33 inhibitory domain (SEQ ID NO:112). In some embodiments, the iCAR comprises a SIGLEC5 inhibitory domain (SEQ ID NO:113). In some embodiments, the iCAR comprises a SIGLEC6 inhibitory domain (SEQ ID NO:114). In some embodiments, the iCAR comprises a SIGLEC7 inhibitory domain (SEQ ID NO:115). In some embodiments, the iCAR comprises a SIGLEC8 inhibitory domain (SEQ ID NO:116). In some embodiments, the iCAR comprises a SIGLEC9 inhibitory domain (SEQ ID NO:117). In some embodiments, the iCAR comprises a SIGLEC10 inhibitory domain (SEQ ID NO:118). In some embodiments, the iCAR comprises a SIGLEC11 inhibitory domain (SEQ ID NO:119). In some embodiments, the iCAR comprises a SIGLEC12 inhibitory domain (SEQ ID NO:120). In some embodiments, the iCAR comprises a PECAM1/CD31 inhibitory domain (SEQ ID NO:121). In some embodiments, the iCAR comprises a CD200R1 inhibitory domain (SEQ ID NO:122). In some embodiments, the iCAR comprises a FCRL1 inhibitory domain (SEQ ID NO:123). In some embodiments, the iCAR comprises a FCRL2 inhibitory domain (SEQ ID NO:124). In some embodiments, the iCAR comprises a FCRL3 inhibitory domain (SEQ ID NO:125). In some embodiments, the iCAR comprises a FCRL4 inhibitory domain (SEQ ID NO:126). In some embodiments, the iCAR comprises a FCRL5 inhibitory domain (SEQ ID NO:127). In some embodiments, the iCAR comprises a SLAMF1 inhibitory domain (SEQ ID NO:128). In some embodiments, the iCAR comprises a SLAMF5 inhibitory domain (SEQ ID NO:129). In some embodiments, the iCAR comprises a BTLA inhibitory domain (SEQ ID NO:130). In some embodiments, the iCAR comprises a LAG3 inhibitory domain (SEQ ID NO:131). In some embodiments, the iCAR comprises a 2B4 inhibitory domain (SEQ ID NO:132). In some embodiments, the iCAR comprises a CD160 inhibitory domain (SEQ ID NO:133). In some embodiments, the iCAR comprises a CEACAM1 inhibitory domain (SEQ ID NO:134). In some embodiments, the iCAR comprises a TIM3 inhibitory domain (SEQ ID NO:135). In some embodiments, the iCAR comprises a VISTA inhibitory domain (SEQ ID NO:136). In some embodiments, the iCAR comprises a TIGIT inhibitory domain (SEQ ID NO:137). In some embodiments, the iCAR comprises a SIRPalpha inhibitory domain (SEQ ID NO:138). In some embodiments, the iCAR comprises a FcγRIIB inhibitory domain (SEQ ID NO:139). In some embodiments, the iCAR comprises a CD5 inhibitory domain (SEQ ID NO:140). In some embodiments, the iCAR comprises a CD300a inhibitory domain (SEQ ID NO:141). In some embodiments, the iCAR comprises a CD300f inhibitory domain (SEQ ID NO:142). In some embodiments, the iCAR comprises a LIR1 inhibitory domain (SEQ ID NO:143). In some embodiments, the iCAR comprises a LIR2 inhibitory domain (SEQ ID NO:144). In some embodiments, the iCAR comprises a LIR3 inhibitory domain (SEQ ID NO:145). In some embodiments, the iCAR comprises a LIR5 inhibitory domain (SEQ ID NO:146). In some embodiments, the iCAR comprises a LIR8 inhibitory domain (SEQ ID NO:147). In some embodiments, the iCAR comprises a Ly9 inhibitory domain (SEQ ID NO:148). In some embodiments, the iCAR comprises a 2×PD1(G4S) inhibitory domain (SEQ ID NO:149). In some embodiments, the iCAR comprises a 2×PD1(PD1) inhibitory domain (SEQ ID NO:150). In some embodiments, the iCAR comprises a PVRIg inhibitory domain (SEQ ID NO:151). In some embodiments, the iCAR comprises an AA2AR inhibitory domain (SEQ ID NO:152). In some embodiments, the iCAR comprises a signal peptide upstream of the iCAR portion, wherein the signal peptide is a CD8 alpha signal peptide (SEQ ID NO: 161), a GM-CSF signal peptide (SEQ ID NO: 162), or a mIgK signal peptide (SEQ ID NO: 306).

In some embodiments, the iCAR comprises an scFv component comprising the VL and VH sequences of Hz.BB7.2 VH1-69_A18VK (SEQ ID NOs: 57 and 58). In some embodiments, the orientation of the iCAR VH and VL regions is VH-VL. In some embodiments, the orientation of the iCAR VH and VL regions is VL-VH. In some embodiments, the iCAR scFv comprises a (G4S)X3 linker (SEQ ID NO:81) linker that covalently connects the VH and the VL to form the iCAR scFv. In some embodiments, the iCAR scFv comprises a Whitlow linker (SEQ ID NO:82) linker that covalently connects the VH and the VL to form the iCAR scFv. In some embodiments, the iCAR comprises a CD8 alpha hinge domain (SEQ ID NO:84). In some embodiments, the iCAR comprises a CD28 hinge domain (SEQ ID NO:85). In some embodiments, the iCAR comprises a PD-1 hinge domain (SEQ ID NO:86). In some embodiments, the iCAR comprises a LIR1 Ig3-4 hinge domain (SEQ ID NO:87). In some embodiments, the iCAR comprises a LIR1 Ig-4 hinge domain (SEQ ID NO:88). In some embodiments, the iCAR comprises a LIR1 52 aa hinge domain (SEQ ID NO:89). In some embodiments, the iCAR comprises a LIR1 36 aa hinge domain (SEQ ID NO:90). In some embodiments, the iCAR comprises a LIR1 30 aa hinge domain (SEQ ID NO:91). In some embodiments, the iCAR comprises a LIR1 8 aa hinge domain (SEQ ID NO:92). In some embodiments, the iCAR comprises a CD33 hinge domain (SEQ ID NO:93). In some embodiments, the iCAR comprises a KIR2DL1 hinge domain (SEQ ID NO:94). In some embodiments, the iCAR comprises a LIR1 26 aa hinge domain (SEQ ID NO: 289). In some embodiments, the iCAR comprises PD-1 (47) hinge domain (SEQ ID NO: 290). In some embodiments, the iCAR comprises PD-1 (42) hinge domain (SEQ ID NO: 291). In some embodiments, the iCAR comprises PD-1 (36) hinge domain (SEQ ID NO: 292). In some embodiments, the iCAR comprises PD-1 (30) hinge domain (SEQ ID NO: 293). In some embodiments, the iCAR comprises PD-1 (26) hinge domain (SEQ ID NO: 294). In some embodiments, the iCAR comprises PD-1 (20) hinge domain (SEQ ID NO: 295). In some embodiments, the iCAR comprises a CD8 alpha transmembrane domain (SEQ ID NO:95). In some embodiments, the iCAR comprises a CD28 transmembrane domain (SEQ ID NO:96). In some embodiments, the iCAR comprises a PD-1 transmembrane domain (SEQ ID NO:97). In some embodiments, the iCAR comprises a LIR1 transmembrane domain (SEQ ID NO:98). In some embodiments, the iCAR comprises a CD33 transmembrane domain (SEQ ID NO:99). In some embodiments, the iCAR comprises a KIR2DL1 transmembrane domain (SEQ ID NO:100). In some embodiments, the iCAR comprises a PD-1 inhibitory domain (SEQ ID NO:101). In some embodiments, the iCAR comprises a KIR2DL1 inhibitory domain (SEQ ID NO:102). In some embodiments, the iCAR comprises a KIR2DL2 inhibitory domain (SEQ ID NO:103). In some embodiments, the iCAR comprises a KIR2DL3 inhibitory domain (SEQ ID NO:104). In some embodiments, the iCAR comprises a KIR2DL4 inhibitory domain (SEQ ID NO:105). In some embodiments, the iCAR comprises a KIR2DL5A inhibitory domain (SEQ ID NO:106). In some embodiments, the iCAR comprises a KIR3DL1 inhibitory domain (SEQ ID NO:107). In some embodiments, the iCAR comprises a KIR3DL2 inhibitory domain (SEQ ID NO:108). In some embodiments, the iCAR comprises a KIR3DL3 inhibitory domain (SEQ ID NO:109). In some embodiments, the iCAR comprises a LAIR1 inhibitory domain (SEQ ID NO:110). In some embodiments, the iCAR comprises a CD22 inhibitory domain (SEQ ID NO:111). In some embodiments, the iCAR comprises a CD33 inhibitory domain (SEQ ID NO:112). In some embodiments, the iCAR comprises a SIGLEC5 inhibitory domain (SEQ ID NO:113). In some embodiments, the iCAR comprises a SIGLEC6 inhibitory domain (SEQ ID NO:114). In some embodiments, the iCAR comprises a SIGLEC7 inhibitory domain (SEQ ID NO:115). In some embodiments, the iCAR comprises a SIGLEC8 inhibitory domain (SEQ ID NO:116). In some embodiments, the iCAR comprises a SIGLEC9 inhibitory domain (SEQ ID NO:117). In some embodiments, the iCAR comprises a SIGLEC10 inhibitory domain (SEQ ID NO:118). In some embodiments, the iCAR comprises a SIGLEC11 inhibitory domain (SEQ ID NO:119). In some embodiments, the iCAR comprises a SIGLEC12 inhibitory domain (SEQ ID NO:120). In some embodiments, the iCAR comprises a PECAM1/CD31 inhibitory domain (SEQ ID NO:121). In some embodiments, the iCAR comprises a CD200R1 inhibitory domain (SEQ ID NO:122). In some embodiments, the iCAR comprises a FCRL1 inhibitory domain (SEQ ID NO:123). In some embodiments, the iCAR comprises a FCRL2 inhibitory domain (SEQ ID NO:124). In some embodiments, the iCAR comprises a FCRL3 inhibitory domain (SEQ ID NO:125). In some embodiments, the iCAR comprises a FCRL4 inhibitory domain (SEQ ID NO:126). In some embodiments, the iCAR comprises a FCRL5 inhibitory domain (SEQ ID NO:127). In some embodiments, the iCAR comprises a SLAMF1 inhibitory domain (SEQ ID NO:128). In some embodiments, the iCAR comprises a SLAMF5 inhibitory domain (SEQ ID NO:129). In some embodiments, the iCAR comprises a BTLA inhibitory domain (SEQ ID NO:130). In some embodiments, the iCAR comprises a LAG3 inhibitory domain (SEQ ID NO:131). In some embodiments, the iCAR comprises a 2B4 inhibitory domain (SEQ ID NO:132). In some embodiments, the iCAR comprises a CD160 inhibitory domain (SEQ ID NO:133). In some embodiments, the iCAR comprises a CEACAM1 inhibitory domain (SEQ ID NO:134). In some embodiments, the iCAR comprises a TIM3 inhibitory domain (SEQ ID NO:135). In some embodiments, the iCAR comprises a VISTA inhibitory domain (SEQ ID NO:136). In some embodiments, the iCAR comprises a TIGIT inhibitory domain (SEQ ID NO:137). In some embodiments, the iCAR comprises a SIRPalpha inhibitory domain (SEQ ID NO:138). In some embodiments, the iCAR comprises a FcγRIIB inhibitory domain (SEQ ID NO:139). In some embodiments, the iCAR comprises a CD5 inhibitory domain (SEQ ID NO:140). In some embodiments, the iCAR comprises a CD300a inhibitory domain (SEQ ID NO:141). In some embodiments, the iCAR comprises a CD300f inhibitory domain (SEQ ID NO:142). In some embodiments, the iCAR comprises a LIR1 inhibitory domain (SEQ ID NO:143). In some embodiments, the iCAR comprises a LIR2 inhibitory domain (SEQ ID NO:144). In some embodiments, the iCAR comprises a LIR3 inhibitory domain (SEQ ID NO:145). In some embodiments, the iCAR comprises a LIR5 inhibitory domain (SEQ ID NO:146). In some embodiments, the iCAR comprises a LIR8 inhibitory domain (SEQ ID NO:147). In some embodiments, the iCAR comprises a Ly9 inhibitory domain (SEQ ID NO:148). In some embodiments, the iCAR comprises a 2×PD1(G4S) inhibitory domain (SEQ ID NO:149). In some embodiments, the iCAR comprises a 2×PD1(PD1) inhibitory domain (SEQ ID NO:150). In some embodiments, the iCAR comprises a PVRIg inhibitory domain (SEQ ID NO:151). In some embodiments, the iCAR comprises an AA2AR inhibitory domain (SEQ ID NO:152). In some embodiments, the iCAR comprises a signal peptide upstream of the iCAR portion, wherein the signal peptide is a CD8 alpha signal peptide (SEQ ID NO: 161), a GM-CSF signal peptide (SEQ ID NO: 162), or a mIgK signal peptide (SEQ ID NO: 306).

In some embodiments, the iCAR comprises an scFv component comprising the VL and VH sequences of Hz.BB7.2 VH1-69 (27,30)_A18 (SEQ ID NOs: 59 and 60). In some embodiments, the orientation of the iCAR VH and VL regions is VH-VL. In some embodiments, the orientation of the iCAR VH and VL regions is VL-VH. In some embodiments, the iCAR scFv comprises a (G4S)X3 linker (SEQ ID NO:81) linker that covalently connects the VH and the VL to form the iCAR scFv. In some embodiments, the iCAR scFv comprises a Whitlow linker (SEQ ID NO:82) linker that covalently connects the VH and the VL to form the iCAR scFv. In some embodiments, the iCAR comprises a CD8 alpha hinge domain (SEQ ID NO:84). In some embodiments, the iCAR comprises a CD28 hinge domain (SEQ ID NO:85). In some embodiments, the iCAR comprises a PD-1 hinge domain (SEQ ID NO:86). In some embodiments, the iCAR comprises a LIR1 Ig3-4 hinge domain (SEQ ID NO:87). In some embodiments, the iCAR comprises a LIR1 Ig-4 hinge domain (SEQ ID NO:88). In some embodiments, the iCAR comprises a LIR1 52 aa hinge domain (SEQ ID NO:89). In some embodiments, the iCAR comprises a LIR1 36 aa hinge domain (SEQ ID NO:90). In some embodiments, the iCAR comprises a LIR1 30 aa hinge domain (SEQ ID NO:91). In some embodiments, the iCAR comprises a LIR1 8 aa hinge domain (SEQ ID NO:92). In some embodiments, the iCAR comprises a CD33 hinge domain (SEQ ID NO:93). In some embodiments, the iCAR comprises a KIR2DL1 hinge domain (SEQ ID NO:94). In some embodiments, the iCAR comprises a LIR1 26 aa hinge domain (SEQ ID NO: 289). In some embodiments, the iCAR comprises PD-1 (47) hinge domain (SEQ ID NO: 290). In some embodiments, the iCAR comprises PD-1 (42) hinge domain (SEQ ID NO: 291). In some embodiments, the iCAR comprises PD-1 (36) hinge domain (SEQ ID NO: 292). In some embodiments, the iCAR comprises PD-1 (30) hinge domain (SEQ ID NO: 293). In some embodiments, the iCAR comprises PD-1 (26) hinge domain (SEQ ID NO: 294). In some embodiments, the iCAR comprises PD-1 (20) hinge domain (SEQ ID NO: 295). In some embodiments, the iCAR comprises a CD8 alpha transmembrane domain (SEQ ID NO:95). In some embodiments, the iCAR comprises a CD28 transmembrane domain (SEQ ID NO:96). In some embodiments, the iCAR comprises a PD-1 transmembrane domain (SEQ ID NO:97). In some embodiments, the iCAR comprises a LIR1 transmembrane domain (SEQ ID NO:98). In some embodiments, the iCAR comprises a CD33 transmembrane domain (SEQ ID NO:99). In some embodiments, the iCAR comprises a KIR2DL1 transmembrane domain (SEQ ID NO:100). In some embodiments, the iCAR comprises a PD-1 inhibitory domain (SEQ ID NO:101). In some embodiments, the iCAR comprises a KIR2DL1 inhibitory domain (SEQ ID NO:102). In some embodiments, the iCAR comprises a KIR2DL2 inhibitory domain (SEQ ID NO:103). In some embodiments, the iCAR comprises a KIR2DL3 inhibitory domain (SEQ ID NO:104). In some embodiments, the iCAR comprises a KIR2DL4 inhibitory domain (SEQ ID NO:105). In some embodiments, the iCAR comprises a KIR2DL5A inhibitory domain (SEQ ID NO:106). In some embodiments, the iCAR comprises a KIR3DL1 inhibitory domain (SEQ ID NO:107). In some embodiments, the iCAR comprises a KIR3DL2 inhibitory domain (SEQ ID NO:108). In some embodiments, the iCAR comprises a KIR3DL3 inhibitory domain (SEQ ID NO:109). In some embodiments, the iCAR comprises a LAIR1 inhibitory domain (SEQ ID NO:110). In some embodiments, the iCAR comprises a CD22 inhibitory domain (SEQ ID NO:111). In some embodiments, the iCAR comprises a CD33 inhibitory domain (SEQ ID NO:112). In some embodiments, the iCAR comprises a SIGLEC5 inhibitory domain (SEQ ID NO:113). In some embodiments, the iCAR comprises a SIGLEC6 inhibitory domain (SEQ ID NO:114). In some embodiments, the iCAR comprises a SIGLEC7 inhibitory domain (SEQ ID NO:115). In some embodiments, the iCAR comprises a SIGLEC8 inhibitory domain (SEQ ID NO:116). In some embodiments, the iCAR comprises a SIGLEC9 inhibitory domain (SEQ ID NO:117). In some embodiments, the iCAR comprises a SIGLEC10 inhibitory domain (SEQ ID NO:118). In some embodiments, the iCAR comprises a SIGLEC11 inhibitory domain (SEQ ID NO:119). In some embodiments, the iCAR comprises a SIGLEC12 inhibitory domain (SEQ ID NO:120). In some embodiments, the iCAR comprises a PECAM1/CD31 inhibitory domain (SEQ ID NO:121). In some embodiments, the iCAR comprises a CD200R1 inhibitory domain (SEQ ID NO:122). In some embodiments, the iCAR comprises a FCRL1 inhibitory domain (SEQ ID NO:123). In some embodiments, the iCAR comprises a FCRL2 inhibitory domain (SEQ ID NO:124). In some embodiments, the iCAR comprises a FCRL3 inhibitory domain (SEQ ID NO:125). In some embodiments, the iCAR comprises a FCRL4 inhibitory domain (SEQ ID NO:126). In some embodiments, the iCAR comprises a FCRL5 inhibitory domain (SEQ ID NO:127). In some embodiments, the iCAR comprises a SLAMF1 inhibitory domain (SEQ ID NO:128). In some embodiments, the iCAR comprises a SLAMF5 inhibitory domain (SEQ ID NO:129). In some embodiments, the iCAR comprises a BTLA inhibitory domain (SEQ ID NO:130). In some embodiments, the iCAR comprises a LAG3 inhibitory domain (SEQ ID NO:131). In some embodiments, the iCAR comprises a 2B4 inhibitory domain (SEQ ID NO:132). In some embodiments, the iCAR comprises a CD160 inhibitory domain (SEQ ID NO:133). In some embodiments, the iCAR comprises a CEACAM1 inhibitory domain (SEQ ID NO:134). In some embodiments, the iCAR comprises a TIM3 inhibitory domain (SEQ ID NO:135). In some embodiments, the iCAR comprises a VISTA inhibitory domain (SEQ ID NO:136). In some embodiments, the iCAR comprises a TIGIT inhibitory domain (SEQ ID NO:137). In some embodiments, the iCAR comprises a SIRPalpha inhibitory domain (SEQ ID NO:138). In some embodiments, the iCAR comprises a FcγRIIB inhibitory domain (SEQ ID NO:139). In some embodiments, the iCAR comprises a CD5 inhibitory domain (SEQ ID NO:140). In some embodiments, the iCAR comprises a CD300a inhibitory domain (SEQ ID NO:141). In some embodiments, the iCAR comprises a CD300f inhibitory domain (SEQ ID NO:142). In some embodiments, the iCAR comprises a LIR1 inhibitory domain (SEQ ID NO:143). In some embodiments, the iCAR comprises a LIR2 inhibitory domain (SEQ ID NO:144). In some embodiments, the iCAR comprises a LIR3 inhibitory domain (SEQ ID NO:145). In some embodiments, the iCAR comprises a LIR5 inhibitory domain (SEQ ID NO:146). In some embodiments, the iCAR comprises a LIR8 inhibitory domain (SEQ ID NO:147). In some embodiments, the iCAR comprises a Ly9 inhibitory domain (SEQ ID NO:148). In some embodiments, the iCAR comprises a 2×PD1(G4S) inhibitory domain (SEQ ID NO:149). In some embodiments, the iCAR comprises a 2×PD1(PD1) inhibitory domain (SEQ ID NO:150). In some embodiments, the iCAR comprises a PVRIg inhibitory domain (SEQ ID NO:151). In some embodiments, the iCAR comprises an AA2AR inhibitory domain (SEQ ID NO:152). In some embodiments, the iCAR comprises a signal peptide upstream of the iCAR portion, wherein the signal peptide is a CD8 alpha signal peptide (SEQ ID NO: 161), a GM-CSF signal peptide (SEQ ID NO: 162), or a mIgK signal peptide (SEQ ID NO: 306).

In some embodiments, the iCAR comprises an scFv component comprising the VL and VH sequences of Hz.BB7.2VH1-69 (27,30,48)_A18 (SEQ ID NOs: 61 and 62). In some embodiments, the orientation of the iCAR VH and VL regions is VH-VL. In some embodiments, the orientation of the iCAR VH and VL regions is VL-VH. In some embodiments, the iCAR scFv comprises a (G4S)X3 linker (SEQ ID NO:81) linker that covalently connects the VH and the VL to form the iCAR scFv. In some embodiments, the iCAR scFv comprises a Whitlow linker (SEQ ID NO:82) linker that covalently connects the VH and the VL to form the iCAR scFv. In some embodiments, the iCAR comprises a CD8 alpha hinge domain (SEQ ID NO:84). In some embodiments, the iCAR comprises a CD28 hinge domain (SEQ ID NO:85). In some embodiments, the iCAR comprises a PD-1 hinge domain (SEQ ID NO:86). In some embodiments, the iCAR comprises a LIR1 Ig3-4 hinge domain (SEQ ID NO:87). In some embodiments, the iCAR comprises a LIR1 Ig-4 hinge domain (SEQ ID NO:88). In some embodiments, the iCAR comprises a LIR1 52 aa hinge domain (SEQ ID NO:89). In some embodiments, the iCAR comprises a LIR1 36 aa hinge domain (SEQ ID NO:90). In some embodiments, the iCAR comprises a LIR1 30 aa hinge domain (SEQ ID NO:91). In some embodiments, the iCAR comprises a LIR1 8 aa hinge domain (SEQ ID NO:92). In some embodiments, the iCAR comprises a CD33 hinge domain (SEQ ID NO:93). In some embodiments, the iCAR comprises a KIR2DL1 hinge domain (SEQ ID NO:94). In some embodiments, the iCAR comprises a LIR1 26 aa hinge domain (SEQ ID NO: 289). In some embodiments, the iCAR comprises PD-1 (47) hinge domain (SEQ ID NO: 290). In some embodiments, the iCAR comprises PD-1 (42) hinge domain (SEQ ID NO: 291). In some embodiments, the iCAR comprises PD-1 (36) hinge domain (SEQ ID NO: 292). In some embodiments, the iCAR comprises PD-1 (30) hinge domain (SEQ ID NO: 293). In some embodiments, the iCAR comprises PD-1 (26) hinge domain (SEQ ID NO: 294). In some embodiments, the iCAR comprises PD-1 (20) hinge domain (SEQ ID NO: 295). In some embodiments, the iCAR comprises a CD8 alpha transmembrane domain (SEQ ID NO:95). In some embodiments, the iCAR comprises a CD28 transmembrane domain (SEQ ID NO:96). In some embodiments, the iCAR comprises a PD-1 transmembrane domain (SEQ ID NO:97). In some embodiments, the iCAR comprises a LIR1 transmembrane domain (SEQ ID NO:98). In some embodiments, the iCAR comprises a CD33 transmembrane domain (SEQ ID NO:99). In some embodiments, the iCAR comprises a KIR2DL1 transmembrane domain (SEQ ID NO:100). In some embodiments, the iCAR comprises a PD-1 inhibitory domain (SEQ ID NO:101). In some embodiments, the iCAR comprises a KIR2DL1 inhibitory domain (SEQ ID NO:102). In some embodiments, the iCAR comprises a KIR2DL2 inhibitory domain (SEQ ID NO:103). In some embodiments, the iCAR comprises a KIR2DL3 inhibitory domain (SEQ ID NO:104). In some embodiments, the iCAR comprises a KIR2DL4 inhibitory domain (SEQ ID NO:105). In some embodiments, the iCAR comprises a KIR2DL5A inhibitory domain (SEQ ID NO:106). In some embodiments, the iCAR comprises a KIR3DL1 inhibitory domain (SEQ ID NO:107). In some embodiments, the iCAR comprises a KIR3DL2 inhibitory domain (SEQ ID NO:108). In some embodiments, the iCAR comprises a KIR3DL3 inhibitory domain (SEQ ID NO:109). In some embodiments, the iCAR comprises a LAIR1 inhibitory domain (SEQ ID NO:110). In some embodiments, the iCAR comprises a CD22 inhibitory domain (SEQ ID NO:111). In some embodiments, the iCAR comprises a CD33 inhibitory domain (SEQ ID NO:112). In some embodiments, the iCAR comprises a SIGLEC5 inhibitory domain (SEQ ID NO:113). In some embodiments, the iCAR comprises a SIGLEC6 inhibitory domain (SEQ ID NO:114). In some embodiments, the iCAR comprises a SIGLEC7 inhibitory domain (SEQ ID NO:115). In some embodiments, the iCAR comprises a SIGLEC8 inhibitory domain (SEQ ID NO:116). In some embodiments, the iCAR comprises a SIGLEC9 inhibitory domain (SEQ ID NO:117). In some embodiments, the iCAR comprises a SIGLEC10 inhibitory domain (SEQ ID NO:118). In some embodiments, the iCAR comprises a SIGLEC11 inhibitory domain (SEQ ID NO:119). In some embodiments, the iCAR comprises a SIGLEC12 inhibitory domain (SEQ ID NO:120). In some embodiments, the iCAR comprises a PECAM1/CD31 inhibitory domain (SEQ ID NO:121). In some embodiments, the iCAR comprises a CD200R1 inhibitory domain (SEQ ID NO:122). In some embodiments, the iCAR comprises a FCRL1 inhibitory domain (SEQ ID NO:123). In some embodiments, the iCAR comprises a FCRL2 inhibitory domain (SEQ ID NO:124). In some embodiments, the iCAR comprises a FCRL3 inhibitory domain (SEQ ID NO:125). In some embodiments, the iCAR comprises a FCRL4 inhibitory domain (SEQ ID NO:126). In some embodiments, the iCAR comprises a FCRL5 inhibitory domain (SEQ ID NO:127). In some embodiments, the iCAR comprises a SLAMF1 inhibitory domain (SEQ ID NO:128). In some embodiments, the iCAR comprises a SLAMF5 inhibitory domain (SEQ ID NO:129). In some embodiments, the iCAR comprises a BTLA inhibitory domain (SEQ ID NO:130). In some embodiments, the iCAR comprises a LAG3 inhibitory domain (SEQ ID NO:131). In some embodiments, the iCAR comprises a 2B4 inhibitory domain (SEQ ID NO:132). In some embodiments, the iCAR comprises a CD160 inhibitory domain (SEQ ID NO:133). In some embodiments, the iCAR comprises a CEACAM1 inhibitory domain (SEQ ID NO:134). In some embodiments, the iCAR comprises a TIM3 inhibitory domain (SEQ ID NO:135). In some embodiments, the iCAR comprises a VISTA inhibitory domain (SEQ ID NO:136). In some embodiments, the iCAR comprises a TIGIT inhibitory domain (SEQ ID NO:137). In some embodiments, the iCAR comprises a SIRPalpha inhibitory domain (SEQ ID NO:138). In some embodiments, the iCAR comprises a FcγRIIB inhibitory domain (SEQ ID NO:139). In some embodiments, the iCAR comprises a CD5 inhibitory domain (SEQ ID NO:140). In some embodiments, the iCAR comprises a CD300a inhibitory domain (SEQ ID NO:141). In some embodiments, the iCAR comprises a CD300f inhibitory domain (SEQ ID NO:142). In some embodiments, the iCAR comprises a LIR1 inhibitory domain (SEQ ID NO:143). In some embodiments, the iCAR comprises a LIR2 inhibitory domain (SEQ ID NO:144). In some embodiments, the iCAR comprises a LIR3 inhibitory domain (SEQ ID NO:145). In some embodiments, the iCAR comprises a LIR5 inhibitory domain (SEQ ID NO:146). In some embodiments, the iCAR comprises a LIR8 inhibitory domain (SEQ ID NO:147). In some embodiments, the iCAR comprises a Ly9 inhibitory domain (SEQ ID NO:148). In some embodiments, the iCAR comprises a 2×PD1(G4S) inhibitory domain (SEQ ID NO:149). In some embodiments, the iCAR comprises a 2×PD1(PD1) inhibitory domain (SEQ ID NO:150). In some embodiments, the iCAR comprises a PVRIg inhibitory domain (SEQ ID NO:151). In some embodiments, the iCAR comprises an AA2AR inhibitory domain (SEQ ID NO:152). In some embodiments, the iCAR comprises a signal peptide upstream of the iCAR portion, wherein the signal peptide is a CD8 alpha signal peptide (SEQ ID NO: 161), a GM-CSF signal peptide (SEQ ID NO: 162), or a mIgK signal peptide (SEQ ID NO: 306).

In some embodiments, the iCAR comprises an scFv component comprising the VL and VH sequences of Hz.BB7.2 VH1-69 (27,30,67)_A18 (SEQ ID NOs: 63 and 64). In some embodiments, the orientation of the iCAR VH and VL regions is VH-VL. In some embodiments, the orientation of the iCAR VH and VL regions is VL-VH. In some embodiments, the iCAR scFv comprises a (G4S)X3 linker (SEQ ID NO:81) linker that covalently connects the VH and the VL to form the iCAR scFv. In some embodiments, the iCAR scFv comprises a Whitlow linker (SEQ ID NO:82) linker that covalently connects the VH and the VL to form the iCAR scFv. In some embodiments, the iCAR comprises a CD8 alpha hinge domain (SEQ ID NO:84). In some embodiments, the iCAR comprises a CD28 hinge domain (SEQ ID NO:85). In some embodiments, the iCAR comprises a PD-1 hinge domain (SEQ ID NO:86). In some embodiments, the iCAR comprises a LIR1 Ig3-4 hinge domain (SEQ ID NO:87). In some embodiments, the iCAR comprises a LIR1 Ig-4 hinge domain (SEQ ID NO:88). In some embodiments, the iCAR comprises a LIR1 52 aa hinge domain (SEQ ID NO:89). In some embodiments, the iCAR comprises a LIR1 36 aa hinge domain (SEQ ID NO:90). In some embodiments, the iCAR comprises a LIR1 30 aa hinge domain (SEQ ID NO:91). In some embodiments, the iCAR comprises a LIR1 8 aa hinge domain (SEQ ID NO:92). In some embodiments, the iCAR comprises a CD33 hinge domain (SEQ ID NO:93). In some embodiments, the iCAR comprises a KIR2DL1 hinge domain (SEQ ID NO:94). In some embodiments, the iCAR comprises a LIR1 26 aa hinge domain (SEQ ID NO: 289). In some embodiments, the iCAR comprises PD-1 (47) hinge domain (SEQ ID NO: 290). In some embodiments, the iCAR comprises PD-1 (42) hinge domain (SEQ ID NO: 291). In some embodiments, the iCAR comprises PD-1 (36) hinge domain (SEQ ID NO: 292). In some embodiments, the iCAR comprises PD-1 (30) hinge domain (SEQ ID NO: 293). In some embodiments, the iCAR comprises PD-1 (26) hinge domain (SEQ ID NO: 294). In some embodiments, the iCAR comprises PD-1 (20) hinge domain (SEQ ID NO: 295). In some embodiments, the iCAR comprises a CD8 alpha transmembrane domain (SEQ ID NO:95). In some embodiments, the iCAR comprises a CD28 transmembrane domain (SEQ ID NO:96). In some embodiments, the iCAR comprises a PD-1 transmembrane domain (SEQ ID NO:97). In some embodiments, the iCAR comprises a LIR1 transmembrane domain (SEQ ID NO:98). In some embodiments, the iCAR comprises a CD33 transmembrane domain (SEQ ID NO:99). In some embodiments, the iCAR comprises a KIR2DL1 transmembrane domain (SEQ ID NO:100). In some embodiments, the iCAR comprises a PD-1 inhibitory domain (SEQ ID NO:101). In some embodiments, the iCAR comprises a KIR2DL1 inhibitory domain (SEQ ID NO:102). In some embodiments, the iCAR comprises a KIR2DL2 inhibitory domain (SEQ ID NO:103). In some embodiments, the iCAR comprises a KIR2DL3 inhibitory domain (SEQ ID NO:104). In some embodiments, the iCAR comprises a KIR2DL4 inhibitory domain (SEQ ID NO:105). In some embodiments, the iCAR comprises a KIR2DL5A inhibitory domain (SEQ ID NO:106). In some embodiments, the iCAR comprises a KIR3DL1 inhibitory domain (SEQ ID NO:107). In some embodiments, the iCAR comprises a KIR3DL2 inhibitory domain (SEQ ID NO:108). In some embodiments, the iCAR comprises a KIR3DL3 inhibitory domain (SEQ ID NO:109). In some embodiments, the iCAR comprises a LAIR1 inhibitory domain (SEQ ID NO:110). In some embodiments, the iCAR comprises a CD22 inhibitory domain (SEQ ID NO:111). In some embodiments, the iCAR comprises a CD33 inhibitory domain (SEQ ID NO:112). In some embodiments, the iCAR comprises a SIGLEC5 inhibitory domain (SEQ ID NO:113). In some embodiments, the iCAR comprises a SIGLEC6 inhibitory domain (SEQ ID NO:114). In some embodiments, the iCAR comprises a SIGLEC7 inhibitory domain (SEQ ID NO:115). In some embodiments, the iCAR comprises a SIGLEC8 inhibitory domain (SEQ ID NO:116). In some embodiments, the iCAR comprises a SIGLEC9 inhibitory domain (SEQ ID NO:117). In some embodiments, the iCAR comprises a SIGLEC10 inhibitory domain (SEQ ID NO:118). In some embodiments, the iCAR comprises a SIGLEC11 inhibitory domain (SEQ ID NO:119). In some embodiments, the iCAR comprises a SIGLEC12 inhibitory domain (SEQ ID NO:120). In some embodiments, the iCAR comprises a PECAM1/CD31 inhibitory domain (SEQ ID NO:121). In some embodiments, the iCAR comprises a CD200R1 inhibitory domain (SEQ ID NO:122). In some embodiments, the iCAR comprises a FCRL1 inhibitory domain (SEQ ID NO:123). In some embodiments, the iCAR comprises a FCRL2 inhibitory domain (SEQ ID NO:124). In some embodiments, the iCAR comprises a FCRL3 inhibitory domain (SEQ ID NO:125). In some embodiments, the iCAR comprises a FCRL4 inhibitory domain (SEQ ID NO:126). In some embodiments, the iCAR comprises a FCRL5 inhibitory domain (SEQ ID NO:127). In some embodiments, the iCAR comprises a SLAMF1 inhibitory domain (SEQ ID NO:128). In some embodiments, the iCAR comprises a SLAMF5 inhibitory domain (SEQ ID NO:129). In some embodiments, the iCAR comprises a BTLA inhibitory domain (SEQ ID NO:130). In some embodiments, the iCAR comprises a LAG3 inhibitory domain (SEQ ID NO:131). In some embodiments, the iCAR comprises a 2B4 inhibitory domain (SEQ ID NO:132). In some embodiments, the iCAR comprises a CD160 inhibitory domain (SEQ ID NO:133). In some embodiments, the iCAR comprises a CEACAM1 inhibitory domain (SEQ ID NO:134). In some embodiments, the iCAR comprises a TIM3 inhibitory domain (SEQ ID NO:135). In some embodiments, the iCAR comprises a VISTA inhibitory domain (SEQ ID NO:136). In some embodiments, the iCAR comprises a TIGIT inhibitory domain (SEQ ID NO:137). In some embodiments, the iCAR comprises a SIRPalpha inhibitory domain (SEQ ID NO:138). In some embodiments, the iCAR comprises a FcγRIIB inhibitory domain (SEQ ID NO:139). In some embodiments, the iCAR comprises a CD5 inhibitory domain (SEQ ID NO:140). In some embodiments, the iCAR comprises a CD300a inhibitory domain (SEQ ID NO:141). In some embodiments, the iCAR comprises a CD300f inhibitory domain (SEQ ID NO:142). In some embodiments, the iCAR comprises a LIR1 inhibitory domain (SEQ ID NO:143). In some embodiments, the iCAR comprises a LIR2 inhibitory domain (SEQ ID NO:144). In some embodiments, the iCAR comprises a LIR3 inhibitory domain (SEQ ID NO:145). In some embodiments, the iCAR comprises a LIR5 inhibitory domain (SEQ ID NO:146). In some embodiments, the iCAR comprises a LIR8 inhibitory domain (SEQ ID NO:147). In some embodiments, the iCAR comprises a Ly9 inhibitory domain (SEQ ID NO:148). In some embodiments, the iCAR comprises a 2×PD1(G4S) inhibitory domain (SEQ ID NO:149). In some embodiments, the iCAR comprises a 2×PD1(PD1) inhibitory domain (SEQ ID NO:150). In some embodiments, the iCAR comprises a PVRIg inhibitory domain (SEQ ID NO:151). In some embodiments, the iCAR comprises an AA2AR inhibitory domain (SEQ ID NO:152). In some embodiments, the iCAR comprises a signal peptide upstream of the iCAR portion, wherein the signal peptide is a CD8 alpha signal peptide (SEQ ID NO: 161), a GM-CSF signal peptide (SEQ ID NO: 162), or a mIgK signal peptide (SEQ ID NO: 306).

In some embodiments, the iCAR comprises an scFv component comprising the VL and VH sequences of Hz.BB7.2 VH1-69 (27,30,69)_A18 (SEQ ID NOs: 65 and 66). In some embodiments, the orientation of the iCAR VH and VL regions is VH-VL. In some embodiments, the orientation of the iCAR VH and VL regions is VL-VH. In some embodiments, the iCAR scFv comprises a (G4S)X3 linker (SEQ ID NO:81) linker that covalently connects the VH and the VL to form the iCAR scFv. In some embodiments, the iCAR scFv comprises a Whitlow linker (SEQ ID NO:82) linker that covalently connects the VH and the VL to form the iCAR scFv. In some embodiments, the iCAR comprises a CD8 alpha hinge domain (SEQ ID NO:84). In some embodiments, the iCAR comprises a CD28 hinge domain (SEQ ID NO:85). In some embodiments, the iCAR comprises a PD-1 hinge domain (SEQ ID NO:86). In some embodiments, the iCAR comprises a LIR1 Ig3-4 hinge domain (SEQ ID NO:87). In some embodiments, the iCAR comprises a LIR1 Ig-4 hinge domain (SEQ ID NO:88). In some embodiments, the iCAR comprises a LIR1 52 aa hinge domain (SEQ ID NO:89). In some embodiments, the iCAR comprises a LIR1 36 aa hinge domain (SEQ ID NO:90). In some embodiments, the iCAR comprises a LIR1 30 aa hinge domain (SEQ ID NO:91). In some embodiments, the iCAR comprises a LIR1 8 aa hinge domain (SEQ ID NO:92). In some embodiments, the iCAR comprises a CD33 hinge domain (SEQ ID NO:93). In some embodiments, the iCAR comprises a KIR2DL1 hinge domain (SEQ ID NO:94). In some embodiments, the iCAR comprises a LIR1 26 aa hinge domain (SEQ ID NO: 289). In some embodiments, the iCAR comprises PD-1 (47) hinge domain (SEQ ID NO: 290). In some embodiments, the iCAR comprises PD-1 (42) hinge domain (SEQ ID NO: 291). In some embodiments, the iCAR comprises PD-1 (36) hinge domain (SEQ ID NO: 292). In some embodiments, the iCAR comprises PD-1 (30) hinge domain (SEQ ID NO: 293). In some embodiments, the iCAR comprises PD-1 (26) hinge domain (SEQ ID NO: 294). In some embodiments, the iCAR comprises PD-1 (20) hinge domain (SEQ ID NO: 295). In some embodiments, the iCAR comprises a CD8 alpha transmembrane domain (SEQ ID NO:95). In some embodiments, the iCAR comprises a CD28 transmembrane domain (SEQ ID NO:96). In some embodiments, the iCAR comprises a PD-1 transmembrane domain (SEQ ID NO:97). In some embodiments, the iCAR comprises a LIR1 transmembrane domain (SEQ ID NO:98). In some embodiments, the iCAR comprises a CD33 transmembrane domain (SEQ ID NO:99). In some embodiments, the iCAR comprises a KIR2DL1 transmembrane domain (SEQ ID NO:100). In some embodiments, the iCAR comprises a PD-1 inhibitory domain (SEQ ID NO:101). In some embodiments, the iCAR comprises a KIR2DL1 inhibitory domain (SEQ ID NO:102). In some embodiments, the iCAR comprises a KIR2DL2 inhibitory domain (SEQ ID NO:103). In some embodiments, the iCAR comprises a KIR2DL3 inhibitory domain (SEQ ID NO:104). In some embodiments, the iCAR comprises a KIR2DL4 inhibitory domain (SEQ ID NO:105). In some embodiments, the iCAR comprises a KIR2DL5A inhibitory domain (SEQ ID NO:106). In some embodiments, the iCAR comprises a KIR3DL1 inhibitory domain (SEQ ID NO:107). In some embodiments, the iCAR comprises a KIR3DL2 inhibitory domain (SEQ ID NO:108). In some embodiments, the iCAR comprises a KIR3DL3 inhibitory domain (SEQ ID NO:109). In some embodiments, the iCAR comprises a LAIR1 inhibitory domain (SEQ ID NO:110). In some embodiments, the iCAR comprises a CD22 inhibitory domain (SEQ ID NO:111). In some embodiments, the iCAR comprises a CD33 inhibitory domain (SEQ ID NO:112). In some embodiments, the iCAR comprises a SIGLEC5 inhibitory domain (SEQ ID NO:113). In some embodiments, the iCAR comprises a SIGLEC6 inhibitory domain (SEQ ID NO:114). In some embodiments, the iCAR comprises a SIGLEC7 inhibitory domain (SEQ ID NO:115). In some embodiments, the iCAR comprises a SIGLEC8 inhibitory domain (SEQ ID NO:116). In some embodiments, the iCAR comprises a SIGLEC9 inhibitory domain (SEQ ID NO:117). In some embodiments, the iCAR comprises a SIGLEC10 inhibitory domain (SEQ ID NO:118). In some embodiments, the iCAR comprises a SIGLEC11 inhibitory domain (SEQ ID NO:119). In some embodiments, the iCAR comprises a SIGLEC12 inhibitory domain (SEQ ID NO:120). In some embodiments, the iCAR comprises a PECAM1/CD31 inhibitory domain (SEQ ID NO:121). In some embodiments, the iCAR comprises a CD200R1 inhibitory domain (SEQ ID NO:122). In some embodiments, the iCAR comprises a FCRL1 inhibitory domain (SEQ ID NO:123). In some embodiments, the iCAR comprises a FCRL2 inhibitory domain (SEQ ID NO:124). In some embodiments, the iCAR comprises a FCRL3 inhibitory domain (SEQ ID NO:125). In some embodiments, the iCAR comprises a FCRL4 inhibitory domain (SEQ ID NO:126). In some embodiments, the iCAR comprises a FCRL5 inhibitory domain (SEQ ID NO:127). In some embodiments, the iCAR comprises a SLAMF1 inhibitory domain (SEQ ID NO:128). In some embodiments, the iCAR comprises a SLAMF5 inhibitory domain (SEQ ID NO:129). In some embodiments, the iCAR comprises a BTLA inhibitory domain (SEQ ID NO:130). In some embodiments, the iCAR comprises a LAG3 inhibitory domain (SEQ ID NO:131). In some embodiments, the iCAR comprises a 2B4 inhibitory domain (SEQ ID NO:132). In some embodiments, the iCAR comprises a CD160 inhibitory domain (SEQ ID NO:133). In some embodiments, the iCAR comprises a CEACAM1 inhibitory domain (SEQ ID NO:134). In some embodiments, the iCAR comprises a TIM3 inhibitory domain (SEQ ID NO:135). In some embodiments, the iCAR comprises a VISTA inhibitory domain (SEQ ID NO:136). In some embodiments, the iCAR comprises a TIGIT inhibitory domain (SEQ ID NO:137). In some embodiments, the iCAR comprises a SIRPalpha inhibitory domain (SEQ ID NO:138). In some embodiments, the iCAR comprises a FcγRIIB inhibitory domain (SEQ ID NO:139). In some embodiments, the iCAR comprises a CD5 inhibitory domain (SEQ ID NO:140). In some embodiments, the iCAR comprises a CD300a inhibitory domain (SEQ ID NO:141). In some embodiments, the iCAR comprises a CD300f inhibitory domain (SEQ ID NO:142). In some embodiments, the iCAR comprises a LIR1 inhibitory domain (SEQ ID NO:143). In some embodiments, the iCAR comprises a LIR2 inhibitory domain (SEQ ID NO:144). In some embodiments, the iCAR comprises a LIR3 inhibitory domain (SEQ ID NO:145). In some embodiments, the iCAR comprises a LIR5 inhibitory domain (SEQ ID NO:146). In some embodiments, the iCAR comprises a LIR8 inhibitory domain (SEQ ID NO:147). In some embodiments, the iCAR comprises a Ly9 inhibitory domain (SEQ ID NO:148). In some embodiments, the iCAR comprises a 2×PD1(G4S) inhibitory domain (SEQ ID NO:149). In some embodiments, the iCAR comprises a 2×PD1(PD1) inhibitory domain (SEQ ID NO:150). In some embodiments, the iCAR comprises a PVRIg inhibitory domain (SEQ ID NO:151). In some embodiments, the iCAR comprises an AA2AR inhibitory domain (SEQ ID NO:152). In some embodiments, the iCAR comprises a signal peptide upstream of the iCAR portion, wherein the signal peptide is a CD8 alpha signal peptide (SEQ ID NO: 161), a GM-CSF signal peptide (SEQ ID NO: 162), or a mIgK signal peptide (SEQ ID NO: 306).

In some embodiments, the iCAR comprises an scFv component comprising the VL and VH sequences of Hz.BB7.2 VH1-69 (27,30,67,69) A18 (SEQ ID NOs: 67 and 68). In some embodiments, the orientation of the iCAR VH and VL regions is VH-VL. In some embodiments, the orientation of the iCAR VH and VL regions is VL-VH. In some embodiments, the iCAR scFv comprises a (G4S)X3 linker (SEQ ID NO:81) linker that covalently connects the VH and the VL to form the iCAR scFv. In some embodiments, the iCAR scFv comprises a Whitlow linker (SEQ ID NO:82) linker that covalently connects the VH and the VL to form the iCAR scFv. In some embodiments, the iCAR comprises a CD8 alpha hinge domain (SEQ ID NO:84). In some embodiments, the iCAR comprises a CD28 hinge domain (SEQ ID NO:85). In some embodiments, the iCAR comprises a PD-1 hinge domain (SEQ ID NO:86). In some embodiments, the iCAR comprises a LIR1 Ig3-4 hinge domain (SEQ ID NO:87). In some embodiments, the iCAR comprises a LIR1 Ig-4 hinge domain (SEQ ID NO:88). In some embodiments, the iCAR comprises a LIR1 52 aa hinge domain (SEQ ID NO:89). In some embodiments, the iCAR comprises a LIR1 36 aa hinge domain (SEQ ID NO:90). In some embodiments, the iCAR comprises a LIR1 30 aa hinge domain (SEQ ID NO:91). In some embodiments, the iCAR comprises a LIR1 8 aa hinge domain (SEQ ID NO:92). In some embodiments, the iCAR comprises a CD33 hinge domain (SEQ ID NO:93). In some embodiments, the iCAR comprises a KIR2DL1 hinge domain (SEQ ID NO:94). In some embodiments, the iCAR comprises a LIR1 26 aa hinge domain (SEQ ID NO: 289). In some embodiments, the iCAR comprises PD-1 (47) hinge domain (SEQ ID NO: 290). In some embodiments, the iCAR comprises PD-1 (42) hinge domain (SEQ ID NO: 291). In some embodiments, the iCAR comprises PD-1 (36) hinge domain (SEQ ID NO: 292). In some embodiments, the iCAR comprises PD-1 (30) hinge domain (SEQ ID NO: 293). In some embodiments, the iCAR comprises PD-1 (26) hinge domain (SEQ ID NO: 294). In some embodiments, the iCAR comprises PD-1 (20) hinge domain (SEQ ID NO: 295). In some embodiments, the iCAR comprises a CD8 alpha transmembrane domain (SEQ ID NO:95). In some embodiments, the iCAR comprises a CD28 transmembrane domain (SEQ ID NO:96). In some embodiments, the iCAR comprises a PD-1 transmembrane domain (SEQ ID NO:97). In some embodiments, the iCAR comprises a LIR1 transmembrane domain (SEQ ID NO:98). In some embodiments, the iCAR comprises a CD33 transmembrane domain (SEQ ID NO:99). In some embodiments, the iCAR comprises a KIR2DL1 transmembrane domain (SEQ ID NO:100). In some embodiments, the iCAR comprises a PD-1 inhibitory domain (SEQ ID NO:101). In some embodiments, the iCAR comprises a KIR2DL1 inhibitory domain (SEQ ID NO:102). In some embodiments, the iCAR comprises a KIR2DL2 inhibitory domain (SEQ ID NO:103). In some embodiments, the iCAR comprises a KIR2DL3 inhibitory domain (SEQ ID NO:104). In some embodiments, the iCAR comprises a KIR2DL4 inhibitory domain (SEQ ID NO:105). In some embodiments, the iCAR comprises a KIR2DL5A inhibitory domain (SEQ ID NO:106). In some embodiments, the iCAR comprises a KIR3DL1 inhibitory domain (SEQ ID NO:107). In some embodiments, the iCAR comprises a KIR3DL2 inhibitory domain (SEQ ID NO:108). In some embodiments, the iCAR comprises a KIR3DL3 inhibitory domain (SEQ ID NO:109). In some embodiments, the iCAR comprises a LAIR1 inhibitory domain (SEQ ID NO:110). In some embodiments, the iCAR comprises a CD22 inhibitory domain (SEQ ID NO:111). In some embodiments, the iCAR comprises a CD33 inhibitory domain (SEQ ID NO:112). In some embodiments, the iCAR comprises a SIGLEC5 inhibitory domain (SEQ ID NO:113). In some embodiments, the iCAR comprises a SIGLEC6 inhibitory domain (SEQ ID NO:114). In some embodiments, the iCAR comprises a SIGLEC7 inhibitory domain (SEQ ID NO:115). In some embodiments, the iCAR comprises a SIGLEC8 inhibitory domain (SEQ ID NO:116). In some embodiments, the iCAR comprises a SIGLEC9 inhibitory domain (SEQ ID NO:117). In some embodiments, the iCAR comprises a SIGLEC10 inhibitory domain (SEQ ID NO:118). In some embodiments, the iCAR comprises a SIGLEC11 inhibitory domain (SEQ ID NO:119). In some embodiments, the iCAR comprises a SIGLEC12 inhibitory domain (SEQ ID NO:120). In some embodiments, the iCAR comprises a PECAM1/CD31 inhibitory domain (SEQ ID NO:121). In some embodiments, the iCAR comprises a CD200R1 inhibitory domain (SEQ ID NO:122). In some embodiments, the iCAR comprises a FCRL1 inhibitory domain (SEQ ID NO:123). In some embodiments, the iCAR comprises a FCRL2 inhibitory domain (SEQ ID NO:124). In some embodiments, the iCAR comprises a FCRL3 inhibitory domain (SEQ ID NO:125). In some embodiments, the iCAR comprises a FCRL4 inhibitory domain (SEQ ID NO:126). In some embodiments, the iCAR comprises a FCRL5 inhibitory domain (SEQ ID NO:127). In some embodiments, the iCAR comprises a SLAMF1 inhibitory domain (SEQ ID NO:128). In some embodiments, the iCAR comprises a SLAMF5 inhibitory domain (SEQ ID NO:129). In some embodiments, the iCAR comprises a BTLA inhibitory domain (SEQ ID NO:130). In some embodiments, the iCAR comprises a LAG3 inhibitory domain (SEQ ID NO:131). In some embodiments, the iCAR comprises a 2B4 inhibitory domain (SEQ ID NO:132). In some embodiments, the iCAR comprises a CD160 inhibitory domain (SEQ ID NO:133). In some embodiments, the iCAR comprises a CEACAM1 inhibitory domain (SEQ ID NO:134). In some embodiments, the iCAR comprises a TIM3 inhibitory domain (SEQ ID NO:135). In some embodiments, the iCAR comprises a VISTA inhibitory domain (SEQ ID NO:136). In some embodiments, the iCAR comprises a TIGIT inhibitory domain (SEQ ID NO:137). In some embodiments, the iCAR comprises a SIRPalpha inhibitory domain (SEQ ID NO:138). In some embodiments, the iCAR comprises a FcγRIIB inhibitory domain (SEQ ID NO:139). In some embodiments, the iCAR comprises a CD5 inhibitory domain (SEQ ID NO:140). In some embodiments, the iCAR comprises a CD300a inhibitory domain (SEQ ID NO:141). In some embodiments, the iCAR comprises a CD300f inhibitory domain (SEQ ID NO:142). In some embodiments, the iCAR comprises a LIR1 inhibitory domain (SEQ ID NO:143). In some embodiments, the iCAR comprises a LIR2 inhibitory domain (SEQ ID NO:144). In some embodiments, the iCAR comprises a LIR3 inhibitory domain (SEQ ID NO:145). In some embodiments, the iCAR comprises a LIR5 inhibitory domain (SEQ ID NO:146). In some embodiments, the iCAR comprises a LIR8 inhibitory domain (SEQ ID NO:147). In some embodiments, the iCAR comprises a Ly9 inhibitory domain (SEQ ID NO:148). In some embodiments, the iCAR comprises a 2×PD1(G4S) inhibitory domain (SEQ ID NO:149). In some embodiments, the iCAR comprises a 2×PD1(PD1) inhibitory domain (SEQ ID NO:150). In some embodiments, the iCAR comprises a PVRIg inhibitory domain (SEQ ID NO:151). In some embodiments, the iCAR comprises an AA2AR inhibitory domain (SEQ ID NO:152). In some embodiments, the iCAR comprises a signal peptide upstream of the iCAR portion, wherein the signal peptide is a CD8 alpha signal peptide (SEQ ID NO: 161), a GM-CSF signal peptide (SEQ ID NO: 162), or a mIgK signal peptide (SEQ ID NO: 306).

In some embodiments, the iCAR comprises an scFv component comprising the VL and VH sequences of Hz.BB7.2 VH1-3_A18 (SEQ ID NOs: 69 and 70). In some embodiments, the orientation of the iCAR VH and VL regions is VH-VL. In some embodiments, the orientation of the iCAR VH and VL regions is VL-VH. In some embodiments, the iCAR scFv comprises a (G4S)X3 linker (SEQ ID NO:81) linker that covalently connects the VH and the VL to form the iCAR scFv. In some embodiments, the iCAR scFv comprises a Whitlow linker (SEQ ID NO:82) linker that covalently connects the VH and the VL to form the iCAR scFv. In some embodiments, the iCAR comprises a CD8 alpha hinge domain (SEQ ID NO:84). In some embodiments, the iCAR comprises a CD28 hinge domain (SEQ ID NO:85). In some embodiments, the iCAR comprises a PD-1 hinge domain (SEQ ID NO:86). In some embodiments, the iCAR comprises a LIR1 Ig3-4 hinge domain (SEQ ID NO:87). In some embodiments, the iCAR comprises a LIR1 Ig-4 hinge domain (SEQ ID NO:88). In some embodiments, the iCAR comprises a LIR1 52 aa hinge domain (SEQ ID NO:89). In some embodiments, the iCAR comprises a LIR1 36 aa hinge domain (SEQ ID NO:90). In some embodiments, the iCAR comprises a LIR1 30 aa hinge domain (SEQ ID NO:91). In some embodiments, the iCAR comprises a LIR1 8 aa hinge domain (SEQ ID NO:92). In some embodiments, the iCAR comprises a CD33 hinge domain (SEQ ID NO:93). In some embodiments, the iCAR comprises a KIR2DL1 hinge domain (SEQ ID NO:94). In some embodiments, the iCAR comprises a LIR1 26 aa hinge domain (SEQ ID NO: 289). In some embodiments, the iCAR comprises PD-1 (47) hinge domain (SEQ ID NO: 290). In some embodiments, the iCAR comprises PD-1 (42) hinge domain (SEQ ID NO: 291). In some embodiments, the iCAR comprises PD-1 (36) hinge domain (SEQ ID NO: 292). In some embodiments, the iCAR comprises PD-1 (30) hinge domain (SEQ ID NO: 293). In some embodiments, the iCAR comprises PD-1 (26) hinge domain (SEQ ID NO: 294). In some embodiments, the iCAR comprises PD-1 (20) hinge domain (SEQ ID NO: 295). In some embodiments, the iCAR comprises a CD8 alpha transmembrane domain (SEQ ID NO:95). In some embodiments, the iCAR comprises a CD28 transmembrane domain (SEQ ID NO:96). In some embodiments, the iCAR comprises a PD-1 transmembrane domain (SEQ ID NO:97). In some embodiments, the iCAR comprises a LIR1 transmembrane domain (SEQ ID NO:98). In some embodiments, the iCAR comprises a CD33 transmembrane domain (SEQ ID NO:99). In some embodiments, the iCAR comprises a KIR2DL1 transmembrane domain (SEQ ID NO:100). In some embodiments, the iCAR comprises a PD-1 inhibitory domain (SEQ ID NO:101). In some embodiments, the iCAR comprises a KIR2DL1 inhibitory domain (SEQ ID NO:102). In some embodiments, the iCAR comprises a KIR2DL2 inhibitory domain (SEQ ID NO:103). In some embodiments, the iCAR comprises a KIR2DL3 inhibitory domain (SEQ ID NO:104). In some embodiments, the iCAR comprises a KIR2DL4 inhibitory domain (SEQ ID NO:105). In some embodiments, the iCAR comprises a KIR2DL5A inhibitory domain (SEQ ID NO:106). In some embodiments, the iCAR comprises a KIR3DL1 inhibitory domain (SEQ ID NO:107). In some embodiments, the iCAR comprises a KIR3DL2 inhibitory domain (SEQ ID NO:108). In some embodiments, the iCAR comprises a KIR3DL3 inhibitory domain (SEQ ID NO:109). In some embodiments, the iCAR comprises a LAIR1 inhibitory domain (SEQ ID NO:110). In some embodiments, the iCAR comprises a CD22 inhibitory domain (SEQ ID NO:111). In some embodiments, the iCAR comprises a CD33 inhibitory domain (SEQ ID NO:112). In some embodiments, the iCAR comprises a SIGLEC5 inhibitory domain (SEQ ID NO:113). In some embodiments, the iCAR comprises a SIGLEC6 inhibitory domain (SEQ ID NO:114). In some embodiments, the iCAR comprises a SIGLEC7 inhibitory domain (SEQ ID NO:115). In some embodiments, the iCAR comprises a SIGLEC8 inhibitory domain (SEQ ID NO:116). In some embodiments, the iCAR comprises a SIGLEC9 inhibitory domain (SEQ ID NO:117). In some embodiments, the iCAR comprises a SIGLEC10 inhibitory domain (SEQ ID NO:118). In some embodiments, the iCAR comprises a SIGLEC11 inhibitory domain (SEQ ID NO:119). In some embodiments, the iCAR comprises a SIGLEC12 inhibitory domain (SEQ ID NO:120). In some embodiments, the iCAR comprises a PECAM1/CD31 inhibitory domain (SEQ ID NO:121). In some embodiments, the iCAR comprises a CD200R1 inhibitory domain (SEQ ID NO:122). In some embodiments, the iCAR comprises a FCRL1 inhibitory domain (SEQ ID NO:123). In some embodiments, the iCAR comprises a FCRL2 inhibitory domain (SEQ ID NO:124). In some embodiments, the iCAR comprises a FCRL3 inhibitory domain (SEQ ID NO:125). In some embodiments, the iCAR comprises a FCRL4 inhibitory domain (SEQ ID NO:126). In some embodiments, the iCAR comprises a FCRL5 inhibitory domain (SEQ ID NO:127). In some embodiments, the iCAR comprises a SLAMF1 inhibitory domain (SEQ ID NO:128). In some embodiments, the iCAR comprises a SLAMF5 inhibitory domain (SEQ ID NO:129). In some embodiments, the iCAR comprises a BTLA inhibitory domain (SEQ ID NO:130). In some embodiments, the iCAR comprises a LAG3 inhibitory domain (SEQ ID NO:131). In some embodiments, the iCAR comprises a 2B4 inhibitory domain (SEQ ID NO:132). In some embodiments, the iCAR comprises a CD160 inhibitory domain (SEQ ID NO:133). In some embodiments, the iCAR comprises a CEACAM1 inhibitory domain (SEQ ID NO:134). In some embodiments, the iCAR comprises a TIM3 inhibitory domain (SEQ ID NO:135). In some embodiments, the iCAR comprises a VISTA inhibitory domain (SEQ ID NO:136). In some embodiments, the iCAR comprises a TIGIT inhibitory domain (SEQ ID NO:137). In some embodiments, the iCAR comprises a SIRPalpha inhibitory domain (SEQ ID NO:138). In some embodiments, the iCAR comprises a FcγRIIB inhibitory domain (SEQ ID NO:139). In some embodiments, the iCAR comprises a CD5 inhibitory domain (SEQ ID NO:140). In some embodiments, the iCAR comprises a CD300a inhibitory domain (SEQ ID NO:141). In some embodiments, the iCAR comprises a CD300f inhibitory domain (SEQ ID NO:142). In some embodiments, the iCAR comprises a LIR1 inhibitory domain (SEQ ID NO:143). In some embodiments, the iCAR comprises a LIR2 inhibitory domain (SEQ ID NO:144). In some embodiments, the iCAR comprises a LIR3 inhibitory domain (SEQ ID NO:145). In some embodiments, the iCAR comprises a LIR5 inhibitory domain (SEQ ID NO:146). In some embodiments, the iCAR comprises a LIR8 inhibitory domain (SEQ ID NO:147). In some embodiments, the iCAR comprises a Ly9 inhibitory domain (SEQ ID NO:148). In some embodiments, the iCAR comprises a 2×PD1(G4S) inhibitory domain (SEQ ID NO:149). In some embodiments, the iCAR comprises a 2×PD1(PD1) inhibitory domain (SEQ ID NO:150). In some embodiments, the iCAR comprises a PVRIg inhibitory domain (SEQ ID NO:151). In some embodiments, the iCAR comprises an AA2AR inhibitory domain (SEQ ID NO:152). In some embodiments, the iCAR comprises a signal peptide upstream of the iCAR portion, wherein the signal peptide is a CD8 alpha signal peptide (SEQ ID NO: 161), a GM-CSF signal peptide (SEQ ID NO: 162), or a mIgK signal peptide (SEQ ID NO: 306).

In some embodiments, the iCAR comprises an scFv component comprising the VL and VH sequences of Hz.BB7.2 VH1-3(48)_A18 (SEQ ID NOs: 71 and 72). In some embodiments, the orientation of the iCAR VH and VL regions is VH-VL. In some embodiments, the orientation of the iCAR VH and VL regions is VL-VH. In some embodiments, the iCAR scFv comprises a (G4S)X3 linker (SEQ ID NO:81) linker that covalently connects the VH and the VL to form the iCAR scFv. In some embodiments, the iCAR scFv comprises a Whitlow linker (SEQ ID NO:82) linker that covalently connects the VH and the VL to form the iCAR scFv. In some embodiments, the iCAR comprises a CD8 alpha hinge domain (SEQ ID NO:84). In some embodiments, the iCAR comprises a CD28 hinge domain (SEQ ID NO:85). In some embodiments, the iCAR comprises a PD-1 hinge domain (SEQ ID NO:86). In some embodiments, the iCAR comprises a LIR1 Ig3-4 hinge domain (SEQ ID NO:87). In some embodiments, the iCAR comprises a LIR1 Ig-4 hinge domain (SEQ ID NO:88). In some embodiments, the iCAR comprises a LIR1 52 aa hinge domain (SEQ ID NO:89). In some embodiments, the iCAR comprises a LIR1 36 aa hinge domain (SEQ ID NO:90). In some embodiments, the iCAR comprises a LIR1 30 aa hinge domain (SEQ ID NO:91). In some embodiments, the iCAR comprises a LIR1 8 aa hinge domain (SEQ ID NO:92). In some embodiments, the iCAR comprises a CD33 hinge domain (SEQ ID NO:93). In some embodiments, the iCAR comprises a KIR2DL1 hinge domain (SEQ ID NO:94). In some embodiments, the iCAR comprises a LIR1 26 aa hinge domain (SEQ ID NO: 289). In some embodiments, the iCAR comprises PD-1 (47) hinge domain (SEQ ID NO: 290). In some embodiments, the iCAR comprises PD-1 (42) hinge domain (SEQ ID NO: 291). In some embodiments, the iCAR comprises PD-1 (36) hinge domain (SEQ ID NO: 292). In some embodiments, the iCAR comprises PD-1 (30) hinge domain (SEQ ID NO: 293). In some embodiments, the iCAR comprises PD-1 (26) hinge domain (SEQ ID NO: 294). In some embodiments, the iCAR comprises PD-1 (20) hinge domain (SEQ ID NO: 295). In some embodiments, the iCAR comprises a CD8 alpha transmembrane domain (SEQ ID NO:95). In some embodiments, the iCAR comprises a CD28 transmembrane domain (SEQ ID NO:96). In some embodiments, the iCAR comprises a PD-1 transmembrane domain (SEQ ID NO:97). In some embodiments, the iCAR comprises a LIR1 transmembrane domain (SEQ ID NO:98). In some embodiments, the iCAR comprises a CD33 transmembrane domain (SEQ ID NO:99). In some embodiments, the iCAR comprises a KIR2DL1 transmembrane domain (SEQ ID NO:100). In some embodiments, the iCAR comprises a PD-1 inhibitory domain (SEQ ID NO:101). In some embodiments, the iCAR comprises a KIR2DL1 inhibitory domain (SEQ ID NO:102). In some embodiments, the iCAR comprises a KIR2DL2 inhibitory domain (SEQ ID NO:103). In some embodiments, the iCAR comprises a KIR2DL3 inhibitory domain (SEQ ID NO:104). In some embodiments, the iCAR comprises a KIR2DL4 inhibitory domain (SEQ ID NO:105). In some embodiments, the iCAR comprises a KIR2DL5A inhibitory domain (SEQ ID NO:106). In some embodiments, the iCAR comprises a KIR3DL1 inhibitory domain (SEQ ID NO:107). In some embodiments, the iCAR comprises a KIR3DL2 inhibitory domain (SEQ ID NO:108). In some embodiments, the iCAR comprises a KIR3DL3 inhibitory domain (SEQ ID NO:109). In some embodiments, the iCAR comprises a LAIR1 inhibitory domain (SEQ ID NO:110). In some embodiments, the iCAR comprises a CD22 inhibitory domain (SEQ ID NO:111). In some embodiments, the iCAR comprises a CD33 inhibitory domain (SEQ ID NO:112). In some embodiments, the iCAR comprises a SIGLEC5 inhibitory domain (SEQ ID NO:113). In some embodiments, the iCAR comprises a SIGLEC6 inhibitory domain (SEQ ID NO:114). In some embodiments, the iCAR comprises a SIGLEC7 inhibitory domain (SEQ ID NO:115). In some embodiments, the iCAR comprises a SIGLEC8 inhibitory domain (SEQ ID NO:116). In some embodiments, the iCAR comprises a SIGLEC9 inhibitory domain (SEQ ID NO:117). In some embodiments, the iCAR comprises a SIGLEC10 inhibitory domain (SEQ ID NO:118). In some embodiments, the iCAR comprises a SIGLEC11 inhibitory domain (SEQ ID NO:119). In some embodiments, the iCAR comprises a SIGLEC12 inhibitory domain (SEQ ID NO:120). In some embodiments, the iCAR comprises a PECAM1/CD31 inhibitory domain (SEQ ID NO:121). In some embodiments, the iCAR comprises a CD200R1 inhibitory domain (SEQ ID NO:122). In some embodiments, the iCAR comprises a FCRL1 inhibitory domain (SEQ ID NO:123). In some embodiments, the iCAR comprises a FCRL2 inhibitory domain (SEQ ID NO:124). In some embodiments, the iCAR comprises a FCRL3 inhibitory domain (SEQ ID NO:125). In some embodiments, the iCAR comprises a FCRL4 inhibitory domain (SEQ ID NO:126). In some embodiments, the iCAR comprises a FCRL5 inhibitory domain (SEQ ID NO:127). In some embodiments, the iCAR comprises a SLAMF1 inhibitory domain (SEQ ID NO:128). In some embodiments, the iCAR comprises a SLAMF5 inhibitory domain (SEQ ID NO:129). In some embodiments, the iCAR comprises a BTLA inhibitory domain (SEQ ID NO:130). In some embodiments, the iCAR comprises a LAG3 inhibitory domain (SEQ ID NO:131). In some embodiments, the iCAR comprises a 2B4 inhibitory domain (SEQ ID NO:132). In some embodiments, the iCAR comprises a CD160 inhibitory domain (SEQ ID NO:133). In some embodiments, the iCAR comprises a CEACAM1 inhibitory domain (SEQ ID NO:134). In some embodiments, the iCAR comprises a TIM3 inhibitory domain (SEQ ID NO:135). In some embodiments, the iCAR comprises a VISTA inhibitory domain (SEQ ID NO:136). In some embodiments, the iCAR comprises a TIGIT inhibitory domain (SEQ ID NO:137). In some embodiments, the iCAR comprises a SIRPalpha inhibitory domain (SEQ ID NO:138). In some embodiments, the iCAR comprises a FcγRIIB inhibitory domain (SEQ ID NO:139). In some embodiments, the iCAR comprises a CD5 inhibitory domain (SEQ ID NO:140). In some embodiments, the iCAR comprises a CD300a inhibitory domain (SEQ ID NO:141). In some embodiments, the iCAR comprises a CD300f inhibitory domain (SEQ ID NO:142). In some embodiments, the iCAR comprises a LIR1 inhibitory domain (SEQ ID NO:143). In some embodiments, the iCAR comprises a LIR2 inhibitory domain (SEQ ID NO:144). In some embodiments, the iCAR comprises a LIR3 inhibitory domain (SEQ ID NO:145). In some embodiments, the iCAR comprises a LIR5 inhibitory domain (SEQ ID NO:146). In some embodiments, the iCAR comprises a LIR8 inhibitory domain (SEQ ID NO:147). In some embodiments, the iCAR comprises a Ly9 inhibitory domain (SEQ ID NO:148). In some embodiments, the iCAR comprises a 2×PD1(G4S) inhibitory domain (SEQ ID NO:149). In some embodiments, the iCAR comprises a 2×PD1(PD1) inhibitory domain (SEQ ID NO:150). In some embodiments, the iCAR comprises a PVRIg inhibitory domain (SEQ ID NO:151). In some embodiments, the iCAR comprises an AA2AR inhibitory domain (SEQ ID NO:152). In some embodiments, the iCAR comprises a signal peptide upstream of the iCAR portion, wherein the signal peptide is a CD8 alpha signal peptide (SEQ ID NO: 161), a GM-CSF signal peptide (SEQ ID NO: 162), or a mIgK signal peptide (SEQ ID NO: 306).

In some embodiments, the iCAR comprises an scFv component comprising the VL and VH sequences of Hz.BB7.2 VH1-3(67)_A18 (SEQ ID NOs: 73 and 74). In some embodiments, the orientation of the iCAR VH and VL regions is VH-VL. In some embodiments, the orientation of the iCAR VH and VL regions is VL-VH. In some embodiments, the iCAR scFv comprises a (G4S)X3 linker (SEQ ID NO:81) linker that covalently connects the VH and the VL to form the iCAR scFv. In some embodiments, the iCAR scFv comprises a Whitlow linker (SEQ ID NO:82) linker that covalently connects the VH and the VL to form the iCAR scFv. In some embodiments, the iCAR comprises a CD8 alpha hinge domain (SEQ ID NO:84). In some embodiments, the iCAR comprises a CD28 hinge domain (SEQ ID NO:85). In some embodiments, the iCAR comprises a PD-1 hinge domain (SEQ ID NO:86). In some embodiments, the iCAR comprises a LIR1 Ig3-4 hinge domain (SEQ ID NO:87). In some embodiments, the iCAR comprises a LIR1 Ig-4 hinge domain (SEQ ID NO:88). In some embodiments, the iCAR comprises a LIR1 52 aa hinge domain (SEQ ID NO:89). In some embodiments, the iCAR comprises a LIR1 36 aa hinge domain (SEQ ID NO:90). In some embodiments, the iCAR comprises a LIR1 30 aa hinge domain (SEQ ID NO:91). In some embodiments, the iCAR comprises a LIR1 8 aa hinge domain (SEQ ID NO:92). In some embodiments, the iCAR comprises a CD33 hinge domain (SEQ ID NO:93). In some embodiments, the iCAR comprises a KIR2DL1 hinge domain (SEQ ID NO:94). In some embodiments, the iCAR comprises a LIR1 26 aa hinge domain (SEQ ID NO: 289). In some embodiments, the iCAR comprises PD-1 (47) hinge domain (SEQ ID NO: 290). In some embodiments, the iCAR comprises PD-1 (42) hinge domain (SEQ ID NO: 291). In some embodiments, the iCAR comprises PD-1 (36) hinge domain (SEQ ID NO: 292). In some embodiments, the iCAR comprises PD-1 (30) hinge domain (SEQ ID NO: 293). In some embodiments, the iCAR comprises PD-1 (26) hinge domain (SEQ ID NO: 294). In some embodiments, the iCAR comprises PD-1 (20) hinge domain (SEQ ID NO: 295). In some embodiments, the iCAR comprises a CD8 alpha transmembrane domain (SEQ ID NO:95). In some embodiments, the iCAR comprises a CD28 transmembrane domain (SEQ ID NO:96). In some embodiments, the iCAR comprises a PD-1 transmembrane domain (SEQ ID NO:97). In some embodiments, the iCAR comprises a LIR1 transmembrane domain (SEQ ID NO:98). In some embodiments, the iCAR comprises a CD33 transmembrane domain (SEQ ID NO:99). In some embodiments, the iCAR comprises a KIR2DL1 transmembrane domain (SEQ ID NO:100). In some embodiments, the iCAR comprises a PD-1 inhibitory domain (SEQ ID NO:101). In some embodiments, the iCAR comprises a KIR2DL1 inhibitory domain (SEQ ID NO:102). In some embodiments, the iCAR comprises a KIR2DL2 inhibitory domain (SEQ ID NO:103). In some embodiments, the iCAR comprises a KIR2DL3 inhibitory domain (SEQ ID NO:104). In some embodiments, the iCAR comprises a KIR2DL4 inhibitory domain (SEQ ID NO:105). In some embodiments, the iCAR comprises a KIR2DL5A inhibitory domain (SEQ ID NO:106). In some embodiments, the iCAR comprises a KIR3DL1 inhibitory domain (SEQ ID NO:107). In some embodiments, the iCAR comprises a KIR3DL2 inhibitory domain (SEQ ID NO:108). In some embodiments, the iCAR comprises a KIR3DL3 inhibitory domain (SEQ ID NO:109). In some embodiments, the iCAR comprises a LAIR1 inhibitory domain (SEQ ID NO:110). In some embodiments, the iCAR comprises a CD22 inhibitory domain (SEQ ID NO:111). In some embodiments, the iCAR comprises a CD33 inhibitory domain (SEQ ID NO:112). In some embodiments, the iCAR comprises a SIGLEC5 inhibitory domain (SEQ ID NO:113). In some embodiments, the iCAR comprises a SIGLEC6 inhibitory domain (SEQ ID NO:114). In some embodiments, the iCAR comprises a SIGLEC7 inhibitory domain (SEQ ID NO:115). In some embodiments, the iCAR comprises a SIGLEC8 inhibitory domain (SEQ ID NO:116). In some embodiments, the iCAR comprises a SIGLEC9 inhibitory domain (SEQ ID NO:117). In some embodiments, the iCAR comprises a SIGLEC10 inhibitory domain (SEQ ID NO:118). In some embodiments, the iCAR comprises a SIGLEC11 inhibitory domain (SEQ ID NO:119). In some embodiments, the iCAR comprises a SIGLEC12 inhibitory domain (SEQ ID NO:120). In some embodiments, the iCAR comprises a PECAM1/CD31 inhibitory domain (SEQ ID NO:121). In some embodiments, the iCAR comprises a CD200R1 inhibitory domain (SEQ ID NO:122). In some embodiments, the iCAR comprises a FCRL1 inhibitory domain (SEQ ID NO:123). In some embodiments, the iCAR comprises a FCRL2 inhibitory domain (SEQ ID NO:124). In some embodiments, the iCAR comprises a FCRL3 inhibitory domain (SEQ ID NO:125). In some embodiments, the iCAR comprises a FCRL4 inhibitory domain (SEQ ID NO:126). In some embodiments, the iCAR comprises a FCRL5 inhibitory domain (SEQ ID NO:127). In some embodiments, the iCAR comprises a SLAMF1 inhibitory domain (SEQ ID NO:128). In some embodiments, the iCAR comprises a SLAMF5 inhibitory domain (SEQ ID NO:129). In some embodiments, the iCAR comprises a BTLA inhibitory domain (SEQ ID NO:130). In some embodiments, the iCAR comprises a LAG3 inhibitory domain (SEQ ID NO:131). In some embodiments, the iCAR comprises a 2B4 inhibitory domain (SEQ ID NO:132). In some embodiments, the iCAR comprises a CD160 inhibitory domain (SEQ ID NO:133). In some embodiments, the iCAR comprises a CEACAM1 inhibitory domain (SEQ ID NO:134). In some embodiments, the iCAR comprises a TIM3 inhibitory domain (SEQ ID NO:135). In some embodiments, the iCAR comprises a VISTA inhibitory domain (SEQ ID NO:136). In some embodiments, the iCAR comprises a TIGIT inhibitory domain (SEQ ID NO:137). In some embodiments, the iCAR comprises a SIRPalpha inhibitory domain (SEQ ID NO:138). In some embodiments, the iCAR comprises a FcγRIIB inhibitory domain (SEQ ID NO:139). In some embodiments, the iCAR comprises a CD5 inhibitory domain (SEQ ID NO:140). In some embodiments, the iCAR comprises a CD300a inhibitory domain (SEQ ID NO:141). In some embodiments, the iCAR comprises a CD300f inhibitory domain (SEQ ID NO:142). In some embodiments, the iCAR comprises a LIR1 inhibitory domain (SEQ ID NO:143). In some embodiments, the iCAR comprises a LIR2 inhibitory domain (SEQ ID NO:144). In some embodiments, the iCAR comprises a LIR3 inhibitory domain (SEQ ID NO:145). In some embodiments, the iCAR comprises a LIR5 inhibitory domain (SEQ ID NO:146). In some embodiments, the iCAR comprises a LIR8 inhibitory domain (SEQ ID NO:147). In some embodiments, the iCAR comprises a Ly9 inhibitory domain (SEQ ID NO:148). In some embodiments, the iCAR comprises a 2×PD1(G4S) inhibitory domain (SEQ ID NO:149). In some embodiments, the iCAR comprises a 2×PD1(PD1) inhibitory domain (SEQ ID NO:150). In some embodiments, the iCAR comprises a PVRIg inhibitory domain (SEQ ID NO:151). In some embodiments, the iCAR comprises an AA2AR inhibitory domain (SEQ ID NO:152). In some embodiments, the iCAR comprises a signal peptide upstream of the iCAR portion, wherein the signal peptide is a CD8 alpha signal peptide (SEQ ID NO: 161), a GM-CSF signal peptide (SEQ ID NO: 162), or a mIgK signal peptide (SEQ ID NO: 306).

In some embodiments, the iCAR comprises an scFv component comprising the VL and VH sequences of Hz.Bb7.2 VH1-3(69)_A18 (SEQ ID NOs: 75 and 76). In some embodiments, the orientation of the iCAR VH and VL regions is VH-VL. In some embodiments, the orientation of the iCAR VH and VL regions is VL-VH. In some embodiments, the iCAR scFv comprises a (G4S)X3 linker (SEQ ID NO:81) linker that covalently connects the VH and the VL to form the iCAR scFv. In some embodiments, the iCAR scFv comprises a Whitlow linker (SEQ ID NO:82) linker that covalently connects the VH and the VL to form the iCAR scFv. In some embodiments, the iCAR comprises a CD8 alpha hinge domain (SEQ ID NO:84). In some embodiments, the iCAR comprises a CD28 hinge domain (SEQ ID NO:85). In some embodiments, the iCAR comprises a PD-1 hinge domain (SEQ ID NO:86). In some embodiments, the iCAR comprises a LIR1 Ig3-4 hinge domain (SEQ ID NO:87). In some embodiments, the iCAR comprises a LIR1 Ig-4 hinge domain (SEQ ID NO:88). In some embodiments, the iCAR comprises a LIR1 52 aa hinge domain (SEQ ID NO:89). In some embodiments, the iCAR comprises a LIR1 36 aa hinge domain (SEQ ID NO:90). In some embodiments, the iCAR comprises a LIR1 30 aa hinge domain (SEQ ID NO:91). In some embodiments, the iCAR comprises a LIR1 8 aa hinge domain (SEQ ID NO:92). In some embodiments, the iCAR comprises a CD33 hinge domain (SEQ ID NO:93). In some embodiments, the iCAR comprises a KIR2DL1 hinge domain (SEQ ID NO:94). In some embodiments, the iCAR comprises a LIR1 26 aa hinge domain (SEQ ID NO: 289). In some embodiments, the iCAR comprises PD-1 (47) hinge domain (SEQ ID NO: 290). In some embodiments, the iCAR comprises PD-1 (42) hinge domain (SEQ ID NO: 291). In some embodiments, the iCAR comprises PD-1 (36) hinge domain (SEQ ID NO: 292). In some embodiments, the iCAR comprises PD-1 (30) hinge domain (SEQ ID NO: 293). In some embodiments, the iCAR comprises PD-1 (26) hinge domain (SEQ ID NO: 294). In some embodiments, the iCAR comprises PD-1 (20) hinge domain (SEQ ID NO: 295). In some embodiments, the iCAR comprises a CD8 alpha transmembrane domain (SEQ ID NO:95). In some embodiments, the iCAR comprises a CD28 transmembrane domain (SEQ ID NO:96). In some embodiments, the iCAR comprises a PD-1 transmembrane domain (SEQ ID NO:97). In some embodiments, the iCAR comprises a LIR1 transmembrane domain (SEQ ID NO:98). In some embodiments, the iCAR comprises a CD33 transmembrane domain (SEQ ID NO:99). In some embodiments, the iCAR comprises a KIR2DL1 transmembrane domain (SEQ ID NO:100). In some embodiments, the iCAR comprises a PD-1 inhibitory domain (SEQ ID NO:101). In some embodiments, the iCAR comprises a KIR2DL1 inhibitory domain (SEQ ID NO:102). In some embodiments, the iCAR comprises a KIR2DL2 inhibitory domain (SEQ ID NO:103). In some embodiments, the iCAR comprises a KIR2DL3 inhibitory domain (SEQ ID NO:104). In some embodiments, the iCAR comprises a KIR2DL4 inhibitory domain (SEQ ID NO:105). In some embodiments, the iCAR comprises a KIR2DL5A inhibitory domain (SEQ ID NO:106). In some embodiments, the iCAR comprises a KIR3DL1 inhibitory domain (SEQ ID NO:107). In some embodiments, the iCAR comprises a KIR3DL2 inhibitory domain (SEQ ID NO:108). In some embodiments, the iCAR comprises a KIR3DL3 inhibitory domain (SEQ ID NO:109). In some embodiments, the iCAR comprises a LAIR1 inhibitory domain (SEQ ID NO:110). In some embodiments, the iCAR comprises a CD22 inhibitory domain (SEQ ID NO:111). In some embodiments, the iCAR comprises a CD33 inhibitory domain (SEQ ID NO:112). In some embodiments, the iCAR comprises a SIGLEC5 inhibitory domain (SEQ ID NO:113). In some embodiments, the iCAR comprises a SIGLEC6 inhibitory domain (SEQ ID NO:114). In some embodiments, the iCAR comprises a SIGLEC7 inhibitory domain (SEQ ID NO:115). In some embodiments, the iCAR comprises a SIGLEC8 inhibitory domain (SEQ ID NO:116). In some embodiments, the iCAR comprises a SIGLEC9 inhibitory domain (SEQ ID NO:117). In some embodiments, the iCAR comprises a SIGLEC10 inhibitory domain (SEQ ID NO:118). In some embodiments, the iCAR comprises a SIGLEC11 inhibitory domain (SEQ ID NO:119). In some embodiments, the iCAR comprises a SIGLEC12 inhibitory domain (SEQ ID NO:120). In some embodiments, the iCAR comprises a PECAM1/CD31 inhibitory domain (SEQ ID NO:121). In some embodiments, the iCAR comprises a CD200R1 inhibitory domain (SEQ ID NO:122). In some embodiments, the iCAR comprises a FCRL1 inhibitory domain (SEQ ID NO:123). In some embodiments, the iCAR comprises a FCRL2 inhibitory domain (SEQ ID NO:124). In some embodiments, the iCAR comprises a FCRL3 inhibitory domain (SEQ ID NO:125). In some embodiments, the iCAR comprises a FCRL4 inhibitory domain (SEQ ID NO:126). In some embodiments, the iCAR comprises a FCRL5 inhibitory domain (SEQ ID NO:127). In some embodiments, the iCAR comprises a SLAMF1 inhibitory domain (SEQ ID NO:128). In some embodiments, the iCAR comprises a SLAMF5 inhibitory domain (SEQ ID NO:129). In some embodiments, the iCAR comprises a BTLA inhibitory domain (SEQ ID NO:130). In some embodiments, the iCAR comprises a LAG3 inhibitory domain (SEQ ID NO:131). In some embodiments, the iCAR comprises a 2B4 inhibitory domain (SEQ ID NO:132). In some embodiments, the iCAR comprises a CD160 inhibitory domain (SEQ ID NO:133). In some embodiments, the iCAR comprises a CEACAM1 inhibitory domain (SEQ ID NO:134). In some embodiments, the iCAR comprises a TIM3 inhibitory domain (SEQ ID NO:135). In some embodiments, the iCAR comprises a VISTA inhibitory domain (SEQ ID NO:136). In some embodiments, the iCAR comprises a TIGIT inhibitory domain (SEQ ID NO:137). In some embodiments, the iCAR comprises a SIRPalpha inhibitory domain (SEQ ID NO:138). In some embodiments, the iCAR comprises a FcγRIIB inhibitory domain (SEQ ID NO:139). In some embodiments, the iCAR comprises a CD5 inhibitory domain (SEQ ID NO:140). In some embodiments, the iCAR comprises a CD300a inhibitory domain (SEQ ID NO:141). In some embodiments, the iCAR comprises a CD300f inhibitory domain (SEQ ID NO:142). In some embodiments, the iCAR comprises a LIR1 inhibitory domain (SEQ ID NO:143). In some embodiments, the iCAR comprises a LIR2 inhibitory domain (SEQ ID NO:144). In some embodiments, the iCAR comprises a LIR3 inhibitory domain (SEQ ID NO:145). In some embodiments, the iCAR comprises a LIR5 inhibitory domain (SEQ ID NO:146). In some embodiments, the iCAR comprises a LIR8 inhibitory domain (SEQ ID NO:147). In some embodiments, the iCAR comprises a Ly9 inhibitory domain (SEQ ID NO:148). In some embodiments, the iCAR comprises a 2×PD1(G4S) inhibitory domain (SEQ ID NO:149). In some embodiments, the iCAR comprises a 2×PD1(PD1) inhibitory domain (SEQ ID NO:150). In some embodiments, the iCAR comprises a PVRIg inhibitory domain (SEQ ID NO:151). In some embodiments, the iCAR comprises an AA2AR inhibitory domain (SEQ ID NO:152). In some embodiments, the iCAR comprises a signal peptide upstream of the iCAR portion, wherein the signal peptide is a CD8 alpha signal peptide (SEQ ID NO: 161), a GM-CSF signal peptide (SEQ ID NO: 162), or a mIgK signal peptide (SEQ ID NO: 306).

In some embodiments, the scFv has the VL and VH sequences of Hz.BB7.2 VH1-3(71)_A18 (SEQ ID NOs: 77 and 78). In some embodiments, the orientation of the iCAR VH and VL regions is VH-VL. In some embodiments, the orientation of the iCAR VH and VL regions is VL-VH. In some embodiments, the iCAR scFv comprises a (G4S)X3 linker (SEQ ID NO:81) linker that covalently connects the VH and the VL to form the iCAR scFv. In some embodiments, the iCAR scFv comprises a Whitlow linker (SEQ ID NO:82) linker that covalently connects the VH and the VL to form the iCAR scFv. In some embodiments, the iCAR comprises a CD8 alpha hinge domain (SEQ ID NO:84). In some embodiments, the iCAR comprises a CD28 hinge domain (SEQ ID NO:85). In some embodiments, the iCAR comprises a PD-1 hinge domain (SEQ ID NO:86). In some embodiments, the iCAR comprises a LIR1 Ig3-4 hinge domain (SEQ ID NO:87). In some embodiments, the iCAR comprises a LIR1 Ig-4 hinge domain (SEQ ID NO:88). In some embodiments, the iCAR comprises a LIR1 52 aa hinge domain (SEQ ID NO:89). In some embodiments, the iCAR comprises a LIR1 36 aa hinge domain (SEQ ID NO:90). In some embodiments, the iCAR comprises a LIR1 30 aa hinge domain (SEQ ID NO:91). In some embodiments, the iCAR comprises a LIR1 8 aa hinge domain (SEQ ID NO:92). In some embodiments, the iCAR comprises a CD33 hinge domain (SEQ ID NO:93). In some embodiments, the iCAR comprises a KIR2DL1 hinge domain (SEQ ID NO:94). In some embodiments, the iCAR comprises a LIR1 26 aa hinge domain (SEQ ID NO: 289). In some embodiments, the iCAR comprises PD-1 (47) hinge domain (SEQ ID NO: 290). In some embodiments, the iCAR comprises PD-1 (42) hinge domain (SEQ ID NO: 291). In some embodiments, the iCAR comprises PD-1 (36) hinge domain (SEQ ID NO: 292). In some embodiments, the iCAR comprises PD-1 (30) hinge domain (SEQ ID NO: 293). In some embodiments, the iCAR comprises PD-1 (26) hinge domain (SEQ ID NO: 294). In some embodiments, the iCAR comprises PD-1 (20) hinge domain (SEQ ID NO: 295). In some embodiments, the iCAR comprises a CD8 alpha transmembrane domain (SEQ ID NO:95). In some embodiments, the iCAR comprises a CD28 transmembrane domain (SEQ ID NO:96). In some embodiments, the iCAR comprises a PD-1 transmembrane domain (SEQ ID NO:97). In some embodiments, the iCAR comprises a LIR1 transmembrane domain (SEQ ID NO:98). In some embodiments, the iCAR comprises a CD33 transmembrane domain (SEQ ID NO:99). In some embodiments, the iCAR comprises a KIR2DL1 transmembrane domain (SEQ ID NO:100). In some embodiments, the iCAR comprises a PD-1 inhibitory domain (SEQ ID NO:101). In some embodiments, the iCAR comprises a KIR2DL1 inhibitory domain (SEQ ID NO:102). In some embodiments, the iCAR comprises a KIR2DL2 inhibitory domain (SEQ ID NO:103). In some embodiments, the iCAR comprises a KIR2DL3 inhibitory domain (SEQ ID NO:104). In some embodiments, the iCAR comprises a KIR2DL4 inhibitory domain (SEQ ID NO:105). In some embodiments, the iCAR comprises a KIR2DL5A inhibitory domain (SEQ ID NO:106). In some embodiments, the iCAR comprises a KIR3DL1 inhibitory domain (SEQ ID NO:107). In some embodiments, the iCAR comprises a KIR3DL2 inhibitory domain (SEQ ID NO:108). In some embodiments, the iCAR comprises a KIR3DL3 inhibitory domain (SEQ ID NO:109). In some embodiments, the iCAR comprises a LAIR1 inhibitory domain (SEQ ID NO:110). In some embodiments, the iCAR comprises a CD22 inhibitory domain (SEQ ID NO:111). In some embodiments, the iCAR comprises a CD33 inhibitory domain (SEQ ID NO:112). In some embodiments, the iCAR comprises a SIGLEC5 inhibitory domain (SEQ ID NO:113). In some embodiments, the iCAR comprises a SIGLEC6 inhibitory domain (SEQ ID NO:114). In some embodiments, the iCAR comprises a SIGLEC7 inhibitory domain (SEQ ID NO:115). In some embodiments, the iCAR comprises a SIGLEC8 inhibitory domain (SEQ ID NO:116). In some embodiments, the iCAR comprises a SIGLEC9 inhibitory domain (SEQ ID NO:117). In some embodiments, the iCAR comprises a SIGLEC10 inhibitory domain (SEQ ID NO:118). In some embodiments, the iCAR comprises a SIGLEC11 inhibitory domain (SEQ ID NO:119). In some embodiments, the iCAR comprises a SIGLEC12 inhibitory domain (SEQ ID NO:120). In some embodiments, the iCAR comprises a PECAM1/CD31 inhibitory domain (SEQ ID NO:121). In some embodiments, the iCAR comprises a CD200R1 inhibitory domain (SEQ ID NO:122). In some embodiments, the iCAR comprises a FCRL1 inhibitory domain (SEQ ID NO:123). In some embodiments, the iCAR comprises a FCRL2 inhibitory domain (SEQ ID NO:124). In some embodiments, the iCAR comprises a FCRL3 inhibitory domain (SEQ ID NO:125). In some embodiments, the iCAR comprises a FCRL4 inhibitory domain (SEQ ID NO:126). In some embodiments, the iCAR comprises a FCRL5 inhibitory domain (SEQ ID NO:127). In some embodiments, the iCAR comprises a SLAMF1 inhibitory domain (SEQ ID NO:128). In some embodiments, the iCAR comprises a SLAMF5 inhibitory domain (SEQ ID NO:129). In some embodiments, the iCAR comprises a BTLA inhibitory domain (SEQ ID NO:130). In some embodiments, the iCAR comprises a LAG3 inhibitory domain (SEQ ID NO:131). In some embodiments, the iCAR comprises a 2B4 inhibitory domain (SEQ ID NO:132). In some embodiments, the iCAR comprises a CD160 inhibitory domain (SEQ ID NO:133). In some embodiments, the iCAR comprises a CEACAM1 inhibitory domain (SEQ ID NO:134). In some embodiments, the iCAR comprises a TIM3 inhibitory domain (SEQ ID NO:135). In some embodiments, the iCAR comprises a VISTA inhibitory domain (SEQ ID NO:136). In some embodiments, the iCAR comprises a TIGIT inhibitory domain (SEQ ID NO:137). In some embodiments, the iCAR comprises a SIRPalpha inhibitory domain (SEQ ID NO:138). In some embodiments, the iCAR comprises a FcγRIIB inhibitory domain (SEQ ID NO:139). In some embodiments, the iCAR comprises a CD5 inhibitory domain (SEQ ID NO:140). In some embodiments, the iCAR comprises a CD300a inhibitory domain (SEQ ID NO:141). In some embodiments, the iCAR comprises a CD300f inhibitory domain (SEQ ID NO:142). In some embodiments, the iCAR comprises a LIR1 inhibitory domain (SEQ ID NO:143). In some embodiments, the iCAR comprises a LIR2 inhibitory domain (SEQ ID NO:144). In some embodiments, the iCAR comprises a LIR3 inhibitory domain (SEQ ID NO:145). In some embodiments, the iCAR comprises a LIR5 inhibitory domain (SEQ ID NO:146). In some embodiments, the iCAR comprises a LIR8 inhibitory domain (SEQ ID NO:147). In some embodiments, the iCAR comprises a Ly9 inhibitory domain (SEQ ID NO:148). In some embodiments, the iCAR comprises a 2×PD1(G4S) inhibitory domain (SEQ ID NO:149). In some embodiments, the iCAR comprises a 2×PD1(PD1) inhibitory domain (SEQ ID NO:150). In some embodiments, the iCAR comprises a PVRIg inhibitory domain (SEQ ID NO:151). In some embodiments, the iCAR comprises an AA2AR inhibitory domain (SEQ ID NO:152). In some embodiments, the iCAR comprises a signal peptide upstream of the iCAR portion, wherein the signal peptide is a CD8 alpha signal peptide (SEQ ID NO: 161), a GM-CSF signal peptide (SEQ ID NO: 162), or a mIgK signal peptide (SEQ ID NO: 306).

In some embodiments, the iCAR comprises an scFv component comprising the VL and VH sequences of Hz. BB7.2VH1-3(73)_A18 (SEQ ID NOs: 79 and 80). In some embodiments, the orientation of the iCAR VH and VL regions is VH-VL. In some embodiments, the orientation of the iCAR VH and VL regions is VL-VH. In some embodiments, the iCAR scFv comprises a (G4S)X3 linker (SEQ ID NO:81) linker that covalently connects the VH and the VL to form the iCAR scFv. In some embodiments, the iCAR scFv comprises a Whitlow linker (SEQ ID NO:82) linker that covalently connects the VH and the VL to form the iCAR scFv. In some embodiments, the iCAR comprises a CD8 alpha hinge domain (SEQ ID NO:84). In some embodiments, the iCAR comprises a CD28 hinge domain (SEQ ID NO:85). In some embodiments, the iCAR comprises a PD-1 hinge domain (SEQ ID NO:86). In some embodiments, the iCAR comprises a LIR1 Ig3-4 hinge domain (SEQ ID NO:87). In some embodiments, the iCAR comprises a LIR1 Ig-4 hinge domain (SEQ ID NO:88). In some embodiments, the iCAR comprises a LIR1 52 aa hinge domain (SEQ ID NO:89). In some embodiments, the iCAR comprises a LIR1 36 aa hinge domain (SEQ ID NO:90). In some embodiments, the iCAR comprises a LIR1 30 aa hinge domain (SEQ ID NO:91). In some embodiments, the iCAR comprises a LIR1 8 aa hinge domain (SEQ ID NO:92). In some embodiments, the iCAR comprises a CD33 hinge domain (SEQ ID NO:93). In some embodiments, the iCAR comprises a KIR2DL1 hinge domain (SEQ ID NO:94). In some embodiments, the iCAR comprises a LIR1 26 aa hinge domain (SEQ ID NO: 289). In some embodiments, the iCAR comprises PD-1 (47) hinge domain (SEQ ID NO: 290). In some embodiments, the iCAR comprises PD-1 (42) hinge domain (SEQ ID NO: 291). In some embodiments, the iCAR comprises PD-1 (36) hinge domain (SEQ ID NO: 292). In some embodiments, the iCAR comprises PD-1 (30) hinge domain (SEQ ID NO: 293). In some embodiments, the iCAR comprises PD-1 (26) hinge domain (SEQ ID NO: 294). In some embodiments, the iCAR comprises PD-1 (20) hinge domain (SEQ ID NO: 295). In some embodiments, the iCAR comprises a CD8 alpha transmembrane domain (SEQ ID NO:95). In some embodiments, the iCAR comprises a CD28 transmembrane domain (SEQ ID NO:96). In some embodiments, the iCAR comprises a PD-1 transmembrane domain (SEQ ID NO:97). In some embodiments, the iCAR comprises a LIR1 transmembrane domain (SEQ ID NO:98). In some embodiments, the iCAR comprises a CD33 transmembrane domain (SEQ ID NO:99). In some embodiments, the iCAR comprises a KIR2DL1 transmembrane domain (SEQ ID NO:100). In some embodiments, the iCAR comprises a PD-1 inhibitory domain (SEQ ID NO:101). In some embodiments, the iCAR comprises a KIR2DL1 inhibitory domain (SEQ ID NO:102). In some embodiments, the iCAR comprises a KIR2DL2 inhibitory domain (SEQ ID NO:103). In some embodiments, the iCAR comprises a KIR2DL3 inhibitory domain (SEQ ID NO:104). In some embodiments, the iCAR comprises a KIR2DL4 inhibitory domain (SEQ ID NO:105). In some embodiments, the iCAR comprises a KIR2DL5A inhibitory domain (SEQ ID NO:106). In some embodiments, the iCAR comprises a KIR3DL1 inhibitory domain (SEQ ID NO:107). In some embodiments, the iCAR comprises a KIR3DL2 inhibitory domain (SEQ ID NO:108). In some embodiments, the iCAR comprises a KIR3DL3 inhibitory domain (SEQ ID NO:109). In some embodiments, the iCAR comprises a LAIR1 inhibitory domain (SEQ ID NO:110). In some embodiments, the iCAR comprises a CD22 inhibitory domain (SEQ ID NO:111). In some embodiments, the iCAR comprises a CD33 inhibitory domain (SEQ ID NO:112). In some embodiments, the iCAR comprises a SIGLEC5 inhibitory domain (SEQ ID NO:113). In some embodiments, the iCAR comprises a SIGLEC6 inhibitory domain (SEQ ID NO:114). In some embodiments, the iCAR comprises a SIGLEC7 inhibitory domain (SEQ ID NO:115). In some embodiments, the iCAR comprises a SIGLEC8 inhibitory domain (SEQ ID NO:116). In some embodiments, the iCAR comprises a SIGLEC9 inhibitory domain (SEQ ID NO:117). In some embodiments, the iCAR comprises a SIGLEC10 inhibitory domain (SEQ ID NO:118). In some embodiments, the iCAR comprises a SIGLEC11 inhibitory domain (SEQ ID NO:119). In some embodiments, the iCAR comprises a SIGLEC12 inhibitory domain (SEQ ID NO:120). In some embodiments, the iCAR comprises a PECAM1/CD31 inhibitory domain (SEQ ID NO:121). In some embodiments, the iCAR comprises a CD200R1 inhibitory domain (SEQ ID NO:122). In some embodiments, the iCAR comprises a FCRL1 inhibitory domain (SEQ ID NO:123). In some embodiments, the iCAR comprises a FCRL2 inhibitory domain (SEQ ID NO:124). In some embodiments, the iCAR comprises a FCRL3 inhibitory domain (SEQ ID NO:125). In some embodiments, the iCAR comprises a FCRL4 inhibitory domain (SEQ ID NO:126). In some embodiments, the iCAR comprises a FCRL5 inhibitory domain (SEQ ID NO:127). In some embodiments, the iCAR comprises a SLAMF1 inhibitory domain (SEQ ID NO:128). In some embodiments, the iCAR comprises a SLAMF5 inhibitory domain (SEQ ID NO:129). In some embodiments, the iCAR comprises a BTLA inhibitory domain (SEQ ID NO:130). In some embodiments, the iCAR comprises a LAG3 inhibitory domain (SEQ ID NO:131). In some embodiments, the iCAR comprises a 2B4 inhibitory domain (SEQ ID NO:132). In some embodiments, the iCAR comprises a CD160 inhibitory domain (SEQ ID NO:133). In some embodiments, the iCAR comprises a CEACAM1 inhibitory domain (SEQ ID NO:134). In some embodiments, the iCAR comprises a TIM3 inhibitory domain (SEQ ID NO:135). In some embodiments, the iCAR comprises a VISTA inhibitory domain (SEQ ID NO:136). In some embodiments, the iCAR comprises a TIGIT inhibitory domain (SEQ ID NO:137). In some embodiments, the iCAR comprises a SIRPalpha inhibitory domain (SEQ ID NO:138). In some embodiments, the iCAR comprises a FcγRIIB inhibitory domain (SEQ ID NO:139). In some embodiments, the iCAR comprises a CD5 inhibitory domain (SEQ ID NO:140). In some embodiments, the iCAR comprises a CD300a inhibitory domain (SEQ ID NO:141). In some embodiments, the iCAR comprises a CD300f inhibitory domain (SEQ ID NO:142). In some embodiments, the iCAR comprises a LIR1 inhibitory domain (SEQ ID NO:143). In some embodiments, the iCAR comprises a LIR2 inhibitory domain (SEQ ID NO:144). In some embodiments, the iCAR comprises a LIR3 inhibitory domain (SEQ ID NO:145). In some embodiments, the iCAR comprises a LIR5 inhibitory domain (SEQ ID NO:146). In some embodiments, the iCAR comprises a LIR8 inhibitory domain (SEQ ID NO:147). In some embodiments, the iCAR comprises a Ly9 inhibitory domain (SEQ ID NO:148). In some embodiments, the iCAR comprises a 2×PD1(G4S) inhibitory domain (SEQ ID NO:149). In some embodiments, the iCAR comprises a 2×PD1(PD1) inhibitory domain (SEQ ID NO:150). In some embodiments, the iCAR comprises a PVRIg inhibitory domain (SEQ ID NO:151). In some embodiments, the iCAR comprises an AA2AR inhibitory domain (SEQ ID NO:152). In some embodiments, the iCAR comprises a signal peptide upstream of the iCAR portion, wherein the signal peptide is a CD8 alpha signal peptide (SEQ ID NO: 161), a GM-CSF signal peptide (SEQ ID NO: 162), or a mIgK signal peptide (SEQ ID NO: 306).

In some embodiments, the scFv has the VL and VH sequences of MWB1.2 (SEQ ID NOs: 163 and 164). In some embodiments, the orientation of the iCAR VH and VL regions is VH-VL. In some embodiments, the orientation of the iCAR VH and VL regions is VL-VH. In some embodiments, the iCAR scFv comprises a (G4S)X3 linker (SEQ ID NO:81) linker that covalently connects the VH and the VL to form the iCAR scFv. In some embodiments, the iCAR scFv comprises a Whitlow linker (SEQ ID NO:82) linker that covalently connects the VH and the VL to form the iCAR scFv. In some embodiments, the iCAR comprises a CD8 alpha hinge domain (SEQ ID NO:84). In some embodiments, the iCAR comprises a CD28 hinge domain (SEQ ID NO:85). In some embodiments, the iCAR comprises a PD-1 hinge domain (SEQ ID NO:86). In some embodiments, the iCAR comprises a LIR1 Ig3-4 hinge domain (SEQ ID NO:87). In some embodiments, the iCAR comprises a LIR1 Ig-4 hinge domain (SEQ ID NO:88). In some embodiments, the iCAR comprises a LIR1 52 aa hinge domain (SEQ ID NO:89). In some embodiments, the iCAR comprises a LIR1 36 aa hinge domain (SEQ ID NO:90). In some embodiments, the iCAR comprises a LIR1 30 aa hinge domain (SEQ ID NO:91). In some embodiments, the iCAR comprises a LIR1 8 aa hinge domain (SEQ ID NO:92). In some embodiments, the iCAR comprises a CD33 hinge domain (SEQ ID NO:93). In some embodiments, the iCAR comprises a KIR2DL1 hinge domain (SEQ ID NO:94). In some embodiments, the iCAR comprises a LIR1 26 aa hinge domain (SEQ ID NO: 289). In some embodiments, the iCAR comprises PD-1 (47) hinge domain (SEQ ID NO: 290). In some embodiments, the iCAR comprises PD-1 (42) hinge domain (SEQ ID NO: 291). In some embodiments, the iCAR comprises PD-1 (36) hinge domain (SEQ ID NO: 292). In some embodiments, the iCAR comprises PD-1 (30) hinge domain (SEQ ID NO: 293). In some embodiments, the iCAR comprises PD-1 (26) hinge domain (SEQ ID NO: 294). In some embodiments, the iCAR comprises PD-1 (20) hinge domain (SEQ ID NO: 295). In some embodiments, the iCAR comprises a CD8 alpha transmembrane domain (SEQ ID NO:95). In some embodiments, the iCAR comprises a CD28 transmembrane domain (SEQ ID NO:96). In some embodiments, the iCAR comprises a PD-1 transmembrane domain (SEQ ID NO:97). In some embodiments, the iCAR comprises a LIR1 transmembrane domain (SEQ ID NO:98). In some embodiments, the iCAR comprises a CD33 transmembrane domain (SEQ ID NO:99). In some embodiments, the iCAR comprises a KIR2DL1 transmembrane domain (SEQ ID NO:100). In some embodiments, the iCAR comprises a PD-1 inhibitory domain (SEQ ID NO:101). In some embodiments, the iCAR comprises a KIR2DL1 inhibitory domain (SEQ ID NO:102). In some embodiments, the iCAR comprises a KIR2DL2 inhibitory domain (SEQ ID NO:103). In some embodiments, the iCAR comprises a KIR2DL3 inhibitory domain (SEQ ID NO:104). In some embodiments, the iCAR comprises a KIR2DL4 inhibitory domain (SEQ ID NO:105). In some embodiments, the iCAR comprises a KIR2DL5A inhibitory domain (SEQ ID NO:106). In some embodiments, the iCAR comprises a KIR3DL1 inhibitory domain (SEQ ID NO:107). In some embodiments, the iCAR comprises a KIR3DL2 inhibitory domain (SEQ ID NO:108). In some embodiments, the iCAR comprises a KIR3DL3 inhibitory domain (SEQ ID NO:109). In some embodiments, the iCAR comprises a LAIR1 inhibitory domain (SEQ ID NO:110). In some embodiments, the iCAR comprises a CD22 inhibitory domain (SEQ ID NO:111). In some embodiments, the iCAR comprises a CD33 inhibitory domain (SEQ ID NO:112). In some embodiments, the iCAR comprises a SIGLEC5 inhibitory domain (SEQ ID NO:113). In some embodiments, the iCAR comprises a SIGLEC6 inhibitory domain (SEQ ID NO:114). In some embodiments, the iCAR comprises a SIGLEC7 inhibitory domain (SEQ ID NO:115). In some embodiments, the iCAR comprises a SIGLEC8 inhibitory domain (SEQ ID NO:116). In some embodiments, the iCAR comprises a SIGLEC9 inhibitory domain (SEQ ID NO:117). In some embodiments, the iCAR comprises a SIGLEC10 inhibitory domain (SEQ ID NO:118). In some embodiments, the iCAR comprises a SIGLEC11 inhibitory domain (SEQ ID NO:119). In some embodiments, the iCAR comprises a SIGLEC12 inhibitory domain (SEQ ID NO:120). In some embodiments, the iCAR comprises a PECAM1/CD31 inhibitory domain (SEQ ID NO:121). In some embodiments, the iCAR comprises a CD200R1 inhibitory domain (SEQ ID NO:122). In some embodiments, the iCAR comprises a FCRL1 inhibitory domain (SEQ ID NO:123). In some embodiments, the iCAR comprises a FCRL2 inhibitory domain (SEQ ID NO:124). In some embodiments, the iCAR comprises a FCRL3 inhibitory domain (SEQ ID NO:125). In some embodiments, the iCAR comprises a FCRL4 inhibitory domain (SEQ ID NO:126). In some embodiments, the iCAR comprises a FCRL5 inhibitory domain (SEQ ID NO:127). In some embodiments, the iCAR comprises a SLAMF1 inhibitory domain (SEQ ID NO:128). In some embodiments, the iCAR comprises a SLAMF5 inhibitory domain (SEQ ID NO:129). In some embodiments, the iCAR comprises a BTLA inhibitory domain (SEQ ID NO:130). In some embodiments, the iCAR comprises a LAG3 inhibitory domain (SEQ ID NO:131). In some embodiments, the iCAR comprises a 2B4 inhibitory domain (SEQ ID NO:132). In some embodiments, the iCAR comprises a CD160 inhibitory domain (SEQ ID NO:133). In some embodiments, the iCAR comprises a CEACAM1 inhibitory domain (SEQ ID NO:134). In some embodiments, the iCAR comprises a TIM3 inhibitory domain (SEQ ID NO:135). In some embodiments, the iCAR comprises a VISTA inhibitory domain (SEQ ID NO:136). In some embodiments, the iCAR comprises a TIGIT inhibitory domain (SEQ ID NO:137). In some embodiments, the iCAR comprises a SIRPalpha inhibitory domain (SEQ ID NO:138). In some embodiments, the iCAR comprises a FcγRIIB inhibitory domain (SEQ ID NO:139). In some embodiments, the iCAR comprises a CD5 inhibitory domain (SEQ ID NO:140). In some embodiments, the iCAR comprises a CD300a inhibitory domain (SEQ ID NO:141). In some embodiments, the iCAR comprises a CD300f inhibitory domain (SEQ ID NO:142). In some embodiments, the iCAR comprises a LIR1 inhibitory domain (SEQ ID NO:143). In some embodiments, the iCAR comprises a LIR2 inhibitory domain (SEQ ID NO:144). In some embodiments, the iCAR comprises a LIR3 inhibitory domain (SEQ ID NO:145). In some embodiments, the iCAR comprises a LIR5 inhibitory domain (SEQ ID NO:146). In some embodiments, the iCAR comprises a LIR8 inhibitory domain (SEQ ID NO:147). In some embodiments, the iCAR comprises a Ly9 inhibitory domain (SEQ ID NO:148). In some embodiments, the iCAR comprises a 2×PD1(G4S) inhibitory domain (SEQ ID NO:149). In some embodiments, the iCAR comprises a 2×PD1(PD1) inhibitory domain (SEQ ID NO:150). In some embodiments, the iCAR comprises a PVRIg inhibitory domain (SEQ ID NO:151). In some embodiments, the iCAR comprises an AA2AR inhibitory domain (SEQ ID NO:152). In some embodiments, the iCAR comprises a signal peptide upstream of the iCAR portion, wherein the signal peptide is a CD8 alpha signal peptide (SEQ ID NO: 161), a GM-CSF signal peptide (SEQ ID NO: 162), or a mIgK signal peptide (SEQ ID NO: 306).

In some embodiments, the scFv has the VL and VH sequences of SN66E3.2 (SEQ ID NOs: 165 and 166). In some embodiments, the orientation of the iCAR VH and VL regions is VH-VL. In some embodiments, the orientation of the iCAR VH and VL regions is VL-VH. In some embodiments, the iCAR scFv comprises a (G4S)X3 linker (SEQ ID NO:81) linker that covalently connects the VH and the VL to form the iCAR scFv. In some embodiments, the iCAR scFv comprises a Whitlow linker (SEQ ID NO:82) linker that covalently connects the VH and the VL to form the iCAR scFv. In some embodiments, the iCAR comprises a CD8 alpha hinge domain (SEQ ID NO:84). In some embodiments, the iCAR comprises a CD28 hinge domain (SEQ ID NO:85). In some embodiments, the iCAR comprises a PD-1 hinge domain (SEQ ID NO:86). In some embodiments, the iCAR comprises a LIR1 Ig3-4 hinge domain (SEQ ID NO:87). In some embodiments, the iCAR comprises a LIR1 Ig-4 hinge domain (SEQ ID NO:88). In some embodiments, the iCAR comprises a LIR1 52 aa hinge domain (SEQ ID NO:89). In some embodiments, the iCAR comprises a LIR1 36 aa hinge domain (SEQ ID NO:90). In some embodiments, the iCAR comprises a LIR1 30 aa hinge domain (SEQ ID NO:91). In some embodiments, the iCAR comprises a LIR1 8 aa hinge domain (SEQ ID NO:92). In some embodiments, the iCAR comprises a CD33 hinge domain (SEQ ID NO:93). In some embodiments, the iCAR comprises a KIR2DL1 hinge domain (SEQ ID NO:94). In some embodiments, the iCAR comprises a LIR1 26 aa hinge domain (SEQ ID NO: 289). In some embodiments, the iCAR comprises PD-1 (47) hinge domain (SEQ ID NO: 290). In some embodiments, the iCAR comprises PD-1 (42) hinge domain (SEQ ID NO: 291). In some embodiments, the iCAR comprises PD-1 (36) hinge domain (SEQ ID NO: 292). In some embodiments, the iCAR comprises PD-1 (30) hinge domain (SEQ ID NO: 293). In some embodiments, the iCAR comprises PD-1 (26) hinge domain (SEQ ID NO: 294). In some embodiments, the iCAR comprises PD-1 (20) hinge domain (SEQ ID NO: 295). In some embodiments, the iCAR comprises a CD8 alpha transmembrane domain (SEQ ID NO:95). In some embodiments, the iCAR comprises a CD28 transmembrane domain (SEQ ID NO:96). In some embodiments, the iCAR comprises a PD-1 transmembrane domain (SEQ ID NO:97). In some embodiments, the iCAR comprises a LIR1 transmembrane domain (SEQ ID NO:98). In some embodiments, the iCAR comprises a CD33 transmembrane domain (SEQ ID NO:99). In some embodiments, the iCAR comprises a KIR2DL1 transmembrane domain (SEQ ID NO:100). In some embodiments, the iCAR comprises a PD-1 inhibitory domain (SEQ ID NO:101). In some embodiments, the iCAR comprises a KIR2DL1 inhibitory domain (SEQ ID NO:102). In some embodiments, the iCAR comprises a KIR2DL2 inhibitory domain (SEQ ID NO:103). In some embodiments, the iCAR comprises a KIR2DL3 inhibitory domain (SEQ ID NO:104). In some embodiments, the iCAR comprises a KIR2DL4 inhibitory domain (SEQ ID NO:105). In some embodiments, the iCAR comprises a KIR2DL5A inhibitory domain (SEQ ID NO:106). In some embodiments, the iCAR comprises a KIR3DL1 inhibitory domain (SEQ ID NO:107). In some embodiments, the iCAR comprises a KIR3DL2 inhibitory domain (SEQ ID NO:108). In some embodiments, the iCAR comprises a KIR3DL3 inhibitory domain (SEQ ID NO:109). In some embodiments, the iCAR comprises a LAIR1 inhibitory domain (SEQ ID NO:110). In some embodiments, the iCAR comprises a CD22 inhibitory domain (SEQ ID NO:111). In some embodiments, the iCAR comprises a CD33 inhibitory domain (SEQ ID NO:112). In some embodiments, the iCAR comprises a SIGLEC5 inhibitory domain (SEQ ID NO:113). In some embodiments, the iCAR comprises a SIGLEC6 inhibitory domain (SEQ ID NO:114). In some embodiments, the iCAR comprises a SIGLEC7 inhibitory domain (SEQ ID NO:115). In some embodiments, the iCAR comprises a SIGLEC8 inhibitory domain (SEQ ID NO:116). In some embodiments, the iCAR comprises a SIGLEC9 inhibitory domain (SEQ ID NO:117). In some embodiments, the iCAR comprises a SIGLEC10 inhibitory domain (SEQ ID NO:118). In some embodiments, the iCAR comprises a SIGLEC11 inhibitory domain (SEQ ID NO:119). In some embodiments, the iCAR comprises a SIGLEC12 inhibitory domain (SEQ ID NO:120). In some embodiments, the iCAR comprises a PECAM1/CD31 inhibitory domain (SEQ ID NO:121). In some embodiments, the iCAR comprises a CD200R1 inhibitory domain (SEQ ID NO:122). In some embodiments, the iCAR comprises a FCRL1 inhibitory domain (SEQ ID NO:123). In some embodiments, the iCAR comprises a FCRL2 inhibitory domain (SEQ ID NO:124). In some embodiments, the iCAR comprises a FCRL3 inhibitory domain (SEQ ID NO:125). In some embodiments, the iCAR comprises a FCRL4 inhibitory domain (SEQ ID NO:126). In some embodiments, the iCAR comprises a FCRL5 inhibitory domain (SEQ ID NO:127). In some embodiments, the iCAR comprises a SLAMF1 inhibitory domain (SEQ ID NO:128). In some embodiments, the iCAR comprises a SLAMF5 inhibitory domain (SEQ ID NO:129). In some embodiments, the iCAR comprises a BTLA inhibitory domain (SEQ ID NO:130). In some embodiments, the iCAR comprises a LAG3 inhibitory domain (SEQ ID NO:131). In some embodiments, the iCAR comprises a 2B4 inhibitory domain (SEQ ID NO:132). In some embodiments, the iCAR comprises a CD160 inhibitory domain (SEQ ID NO:133). In some embodiments, the iCAR comprises a CEACAM1 inhibitory domain (SEQ ID NO:134). In some embodiments, the iCAR comprises a TIM3 inhibitory domain (SEQ ID NO:135). In some embodiments, the iCAR comprises a VISTA inhibitory domain (SEQ ID NO:136). In some embodiments, the iCAR comprises a TIGIT inhibitory domain (SEQ ID NO:137). In some embodiments, the iCAR comprises a SIRPalpha inhibitory domain (SEQ ID NO:138). In some embodiments, the iCAR comprises a FcγRIIB inhibitory domain (SEQ ID NO:139). In some embodiments, the iCAR comprises a CD5 inhibitory domain (SEQ ID NO:140). In some embodiments, the iCAR comprises a CD300a inhibitory domain (SEQ ID NO:141). In some embodiments, the iCAR comprises a CD300f inhibitory domain (SEQ ID NO:142). In some embodiments, the iCAR comprises a LIR1 inhibitory domain (SEQ ID NO:143). In some embodiments, the iCAR comprises a LIR2 inhibitory domain (SEQ ID NO:144). In some embodiments, the iCAR comprises a LIR3 inhibitory domain (SEQ ID NO:145). In some embodiments, the iCAR comprises a LIR5 inhibitory domain (SEQ ID NO:146). In some embodiments, the iCAR comprises a LIR8 inhibitory domain (SEQ ID NO:147). In some embodiments, the iCAR comprises a Ly9 inhibitory domain (SEQ ID NO:148). In some embodiments, the iCAR comprises a 2×PD1(G4S) inhibitory domain (SEQ ID NO:149). In some embodiments, the iCAR comprises a 2×PD1(PD1) inhibitory domain (SEQ ID NO:150). In some embodiments, the iCAR comprises a PVRIg inhibitory domain (SEQ ID NO:151). In some embodiments, the iCAR comprises an AA2AR inhibitory domain (SEQ ID NO:152). In some embodiments, the iCAR comprises a signal peptide upstream of the iCAR portion, wherein the signal peptide is a CD8 alpha signal peptide (SEQ ID NO: 161), a GM-CSF signal peptide (SEQ ID NO: 162), or a mIgK signal peptide (SEQ ID NO: 306).

In some embodiments, the scFv has the VL and VH sequences of MWB1.1 (SEQ ID NOs: 273). In some embodiments, the orientation of the iCAR VH and VL regions is VH-VL. In some embodiments, the orientation of the iCAR VH and VL regions is VL-VH. In some embodiments, the iCAR scFv comprises a (G4S)X3 linker (SEQ ID NO:81) linker that covalently connects the VH and the VL to form the iCAR scFv. In some embodiments, the iCAR scFv comprises a Whitlow linker (SEQ ID NO:82) linker that covalently connects the VH and the VL to form the iCAR scFv. In some embodiments, the iCAR comprises a CD8 alpha hinge domain (SEQ ID NO:84). In some embodiments, the iCAR comprises a CD28 hinge domain (SEQ ID NO:85). In some embodiments, the iCAR comprises a PD-1 hinge domain (SEQ ID NO:86). In some embodiments, the iCAR comprises a LIR1 Ig3-4 hinge domain (SEQ ID NO:87). In some embodiments, the iCAR comprises a LIR1 Ig-4 hinge domain (SEQ ID NO:88). In some embodiments, the iCAR comprises a LIR1 52 aa hinge domain (SEQ ID NO:89). In some embodiments, the iCAR comprises a LIR1 36 aa hinge domain (SEQ ID NO:90). In some embodiments, the iCAR comprises a LIR1 30 aa hinge domain (SEQ ID NO:91). In some embodiments, the iCAR comprises a LIR1 8 aa hinge domain (SEQ ID NO:92). In some embodiments, the iCAR comprises a CD33 hinge domain (SEQ ID NO:93). In some embodiments, the iCAR comprises a KIR2DL1 hinge domain (SEQ ID NO:94). In some embodiments, the iCAR comprises a LIR1 26 aa hinge domain (SEQ ID NO: 289). In some embodiments, the iCAR comprises PD-1 (47) hinge domain (SEQ ID NO: 290). In some embodiments, the iCAR comprises PD-1 (42) hinge domain (SEQ ID NO: 291). In some embodiments, the iCAR comprises PD-1 (36) hinge domain (SEQ ID NO: 292). In some embodiments, the iCAR comprises PD-1 (30) hinge domain (SEQ ID NO: 293). In some embodiments, the iCAR comprises PD-1 (26) hinge domain (SEQ ID NO: 294). In some embodiments, the iCAR comprises PD-1 (20) hinge domain (SEQ ID NO: 295). In some embodiments, the iCAR comprises a CD8 alpha transmembrane domain (SEQ ID NO:95). In some embodiments, the iCAR comprises a CD28 transmembrane domain (SEQ ID NO:96). In some embodiments, the iCAR comprises a PD-1 transmembrane domain (SEQ ID NO:97). In some embodiments, the iCAR comprises a LIR1 transmembrane domain (SEQ ID NO:98). In some embodiments, the iCAR comprises a CD33 transmembrane domain (SEQ ID NO:99). In some embodiments, the iCAR comprises a KIR2DL1 transmembrane domain (SEQ ID NO:100). In some embodiments, the iCAR comprises a PD-1 inhibitory domain (SEQ ID NO:101). In some embodiments, the iCAR comprises a KIR2DL1 inhibitory domain (SEQ ID NO:102). In some embodiments, the iCAR comprises a KIR2DL2 inhibitory domain (SEQ ID NO:103). In some embodiments, the iCAR comprises a KIR2DL3 inhibitory domain (SEQ ID NO:104). In some embodiments, the iCAR comprises a KIR2DL4 inhibitory domain (SEQ ID NO:105). In some embodiments, the iCAR comprises a KIR2DL5A inhibitory domain (SEQ ID NO:106). In some embodiments, the iCAR comprises a KIR3DL1 inhibitory domain (SEQ ID NO:107). In some embodiments, the iCAR comprises a KIR3DL2 inhibitory domain (SEQ ID NO:108). In some embodiments, the iCAR comprises a KIR3DL3 inhibitory domain (SEQ ID NO:109). In some embodiments, the iCAR comprises a LAIR1 inhibitory domain (SEQ ID NO:110). In some embodiments, the iCAR comprises a CD22 inhibitory domain (SEQ ID NO:111). In some embodiments, the iCAR comprises a CD33 inhibitory domain (SEQ ID NO:112). In some embodiments, the iCAR comprises a SIGLEC5 inhibitory domain (SEQ ID NO:113). In some embodiments, the iCAR comprises a SIGLEC6 inhibitory domain (SEQ ID NO:114). In some embodiments, the iCAR comprises a SIGLEC7 inhibitory domain (SEQ ID NO:115). In some embodiments, the iCAR comprises a SIGLEC8 inhibitory domain (SEQ ID NO:116). In some embodiments, the iCAR comprises a SIGLEC9 inhibitory domain (SEQ ID NO:117). In some embodiments, the iCAR comprises a SIGLEC10 inhibitory domain (SEQ ID NO:118). In some embodiments, the iCAR comprises a SIGLEC11 inhibitory domain (SEQ ID NO:119). In some embodiments, the iCAR comprises a SIGLEC12 inhibitory domain (SEQ ID NO:120). In some embodiments, the iCAR comprises a PECAM1/CD31 inhibitory domain (SEQ ID NO:121). In some embodiments, the iCAR comprises a CD200R1 inhibitory domain (SEQ ID NO:122). In some embodiments, the iCAR comprises a FCRL1 inhibitory domain (SEQ ID NO:123). In some embodiments, the iCAR comprises a FCRL2 inhibitory domain (SEQ ID NO:124). In some embodiments, the iCAR comprises a FCRL3 inhibitory domain (SEQ ID NO:125). In some embodiments, the iCAR comprises a FCRL4 inhibitory domain (SEQ ID NO:126). In some embodiments, the iCAR comprises a FCRL5 inhibitory domain (SEQ ID NO:127). In some embodiments, the iCAR comprises a SLAMF1 inhibitory domain (SEQ ID NO:128). In some embodiments, the iCAR comprises a SLAMF5 inhibitory domain (SEQ ID NO:129). In some embodiments, the iCAR comprises a BTLA inhibitory domain (SEQ ID NO:130). In some embodiments, the iCAR comprises a LAG3 inhibitory domain (SEQ ID NO:131). In some embodiments, the iCAR comprises a 2B4 inhibitory domain (SEQ ID NO:132). In some embodiments, the iCAR comprises a CD160 inhibitory domain (SEQ ID NO:133). In some embodiments, the iCAR comprises a CEACAM1 inhibitory domain (SEQ ID NO:134). In some embodiments, the iCAR comprises a TIM3 inhibitory domain (SEQ ID NO:135). In some embodiments, the iCAR comprises a VISTA inhibitory domain (SEQ ID NO:136). In some embodiments, the iCAR comprises a TIGIT inhibitory domain (SEQ ID NO:137). In some embodiments, the iCAR comprises a SIRPalpha inhibitory domain (SEQ ID NO:138). In some embodiments, the iCAR comprises a FcγRIIB inhibitory domain (SEQ ID NO:139). In some embodiments, the iCAR comprises a CD5 inhibitory domain (SEQ ID NO:140). In some embodiments, the iCAR comprises a CD300a inhibitory domain (SEQ ID NO:141). In some embodiments, the iCAR comprises a CD300f inhibitory domain (SEQ ID NO:142). In some embodiments, the iCAR comprises a LIR1 inhibitory domain (SEQ ID NO:143). In some embodiments, the iCAR comprises a LIR2 inhibitory domain (SEQ ID NO:144). In some embodiments, the iCAR comprises a LIR3 inhibitory domain (SEQ ID NO:145). In some embodiments, the iCAR comprises a LIR5 inhibitory domain (SEQ ID NO:146). In some embodiments, the iCAR comprises a LIR8 inhibitory domain (SEQ ID NO:147). In some embodiments, the iCAR comprises a Ly9 inhibitory domain (SEQ ID NO:148). In some embodiments, the iCAR comprises a 2×PD1(G4S) inhibitory domain (SEQ ID NO:149). In some embodiments, the iCAR comprises a 2×PD1(PD1) inhibitory domain (SEQ ID NO:150). In some embodiments, the iCAR comprises a PVRIg inhibitory domain (SEQ ID NO:151). In some embodiments, the iCAR comprises an AA2AR inhibitory domain (SEQ ID NO:152). In some embodiments, the iCAR comprises a signal peptide upstream of the iCAR portion, wherein the signal peptide is a CD8 alpha signal peptide (SEQ ID NO: 161), a GM-CSF signal peptide (SEQ ID NO: 162), or a mIgK signal peptide (SEQ ID NO: 306).

In some embodiments, the scFv has the VL and VH sequences of MWB1.2 (SEQ ID NOs: 274). In some embodiments, the orientation of the iCAR VH and VL regions is VH-VL. In some embodiments, the orientation of the iCAR VH and VL regions is VL-VH. In some embodiments, the iCAR scFv comprises a (G4S)X3 linker (SEQ ID NO:81) linker that covalently connects the VH and the VL to form the iCAR scFv. In some embodiments, the iCAR scFv comprises a Whitlow linker (SEQ ID NO:82) linker that covalently connects the VH and the VL to form the iCAR scFv. In some embodiments, the iCAR comprises a CD8 alpha hinge domain (SEQ ID NO:84). In some embodiments, the iCAR comprises a CD28 hinge domain (SEQ ID NO:85). In some embodiments, the iCAR comprises a PD-1 hinge domain (SEQ ID NO:86). In some embodiments, the iCAR comprises a LIR1 Ig3-4 hinge domain (SEQ ID NO:87). In some embodiments, the iCAR comprises a LIR1 Ig-4 hinge domain (SEQ ID NO:88). In some embodiments, the iCAR comprises a LIR1 52 aa hinge domain (SEQ ID NO:89). In some embodiments, the iCAR comprises a LIR1 36 aa hinge domain (SEQ ID NO:90). In some embodiments, the iCAR comprises a LIR1 30 aa hinge domain (SEQ ID NO:91). In some embodiments, the iCAR comprises a LIR1 8 aa hinge domain (SEQ ID NO:92). In some embodiments, the iCAR comprises a CD33 hinge domain (SEQ ID NO:93). In some embodiments, the iCAR comprises a KIR2DL1 hinge domain (SEQ ID NO:94). In some embodiments, the iCAR comprises a LIR1 26 aa hinge domain (SEQ ID NO: 289). In some embodiments, the iCAR comprises PD-1 (47) hinge domain (SEQ ID NO: 290). In some embodiments, the iCAR comprises PD-1 (42) hinge domain (SEQ ID NO: 291). In some embodiments, the iCAR comprises PD-1 (36) hinge domain (SEQ ID NO: 292). In some embodiments, the iCAR comprises PD-1 (30) hinge domain (SEQ ID NO: 293). In some embodiments, the iCAR comprises PD-1 (26) hinge domain (SEQ ID NO: 294). In some embodiments, the iCAR comprises PD-1 (20) hinge domain (SEQ ID NO: 295). In some embodiments, the iCAR comprises a CD8 alpha transmembrane domain (SEQ ID NO:95). In some embodiments, the iCAR comprises a CD28 transmembrane domain (SEQ ID NO:96). In some embodiments, the iCAR comprises a PD-1 transmembrane domain (SEQ ID NO:97). In some embodiments, the iCAR comprises a LIR1 transmembrane domain (SEQ ID NO:98). In some embodiments, the iCAR comprises a CD33 transmembrane domain (SEQ ID NO:99). In some embodiments, the iCAR comprises a KIR2DL1 transmembrane domain (SEQ ID NO:100). In some embodiments, the iCAR comprises a PD-1 inhibitory domain (SEQ ID NO:101). In some embodiments, the iCAR comprises a KIR2DL1 inhibitory domain (SEQ ID NO:102). In some embodiments, the iCAR comprises a KIR2DL2 inhibitory domain (SEQ ID NO:103). In some embodiments, the iCAR comprises a KIR2DL3 inhibitory domain (SEQ ID NO:104). In some embodiments, the iCAR comprises a KIR2DL4 inhibitory domain (SEQ ID NO:105). In some embodiments, the iCAR comprises a KIR2DL5A inhibitory domain (SEQ ID NO:106). In some embodiments, the iCAR comprises a KIR3DL1 inhibitory domain (SEQ ID NO:107). In some embodiments, the iCAR comprises a KIR3DL2 inhibitory domain (SEQ ID NO:108). In some embodiments, the iCAR comprises a KIR3DL3 inhibitory domain (SEQ ID NO:109). In some embodiments, the iCAR comprises a LAIR1 inhibitory domain (SEQ ID NO:110). In some embodiments, the iCAR comprises a CD22 inhibitory domain (SEQ ID NO:111). In some embodiments, the iCAR comprises a CD33 inhibitory domain (SEQ ID NO:112). In some embodiments, the iCAR comprises a SIGLEC5 inhibitory domain (SEQ ID NO:113). In some embodiments, the iCAR comprises a SIGLEC6 inhibitory domain (SEQ ID NO:114). In some embodiments, the iCAR comprises a SIGLEC7 inhibitory domain (SEQ ID NO:115). In some embodiments, the iCAR comprises a SIGLEC8 inhibitory domain (SEQ ID NO:116). In some embodiments, the iCAR comprises a SIGLEC9 inhibitory domain (SEQ ID NO:117). In some embodiments, the iCAR comprises a SIGLEC10 inhibitory domain (SEQ ID NO:118). In some embodiments, the iCAR comprises a SIGLEC11 inhibitory domain (SEQ ID NO:119). In some embodiments, the iCAR comprises a SIGLEC12 inhibitory domain (SEQ ID NO:120). In some embodiments, the iCAR comprises a PECAM1/CD31 inhibitory domain (SEQ ID NO:121). In some embodiments, the iCAR comprises a CD200R1 inhibitory domain (SEQ ID NO:122). In some embodiments, the iCAR comprises a FCRL1 inhibitory domain (SEQ ID NO:123). In some embodiments, the iCAR comprises a FCRL2 inhibitory domain (SEQ ID NO:124). In some embodiments, the iCAR comprises a FCRL3 inhibitory domain (SEQ ID NO:125). In some embodiments, the iCAR comprises a FCRL4 inhibitory domain (SEQ ID NO:126). In some embodiments, the iCAR comprises a FCRL5 inhibitory domain (SEQ ID NO:127). In some embodiments, the iCAR comprises a SLAMF1 inhibitory domain (SEQ ID NO:128). In some embodiments, the iCAR comprises a SLAMF5 inhibitory domain (SEQ ID NO:129). In some embodiments, the iCAR comprises a BTLA inhibitory domain (SEQ ID NO:130). In some embodiments, the iCAR comprises a LAG3 inhibitory domain (SEQ ID NO:131). In some embodiments, the iCAR comprises a 2B4 inhibitory domain (SEQ ID NO:132). In some embodiments, the iCAR comprises a CD160 inhibitory domain (SEQ ID NO:133). In some embodiments, the iCAR comprises a CEACAM1 inhibitory domain (SEQ ID NO:134). In some embodiments, the iCAR comprises a TIM3 inhibitory domain (SEQ ID NO:135). In some embodiments, the iCAR comprises a VISTA inhibitory domain (SEQ ID NO:136). In some embodiments, the iCAR comprises a TIGIT inhibitory domain (SEQ ID NO:137). In some embodiments, the iCAR comprises a SIRPalpha inhibitory domain (SEQ ID NO:138). In some embodiments, the iCAR comprises a FcγRIIB inhibitory domain (SEQ ID NO:139). In some embodiments, the iCAR comprises a CD5 inhibitory domain (SEQ ID NO:140). In some embodiments, the iCAR comprises a CD300a inhibitory domain (SEQ ID NO:141). In some embodiments, the iCAR comprises a CD300f inhibitory domain (SEQ ID NO:142). In some embodiments, the iCAR comprises a LIR1 inhibitory domain (SEQ ID NO:143). In some embodiments, the iCAR comprises a LIR2 inhibitory domain (SEQ ID NO:144). In some embodiments, the iCAR comprises a LIR3 inhibitory domain (SEQ ID NO:145). In some embodiments, the iCAR comprises a LIR5 inhibitory domain (SEQ ID NO:146). In some embodiments, the iCAR comprises a LIR8 inhibitory domain (SEQ ID NO:147). In some embodiments, the iCAR comprises a Ly9 inhibitory domain (SEQ ID NO:148). In some embodiments, the iCAR comprises a 2×PD1(G4S) inhibitory domain (SEQ ID NO:149). In some embodiments, the iCAR comprises a 2×PD1(PD1) inhibitory domain (SEQ ID NO:150). In some embodiments, the iCAR comprises a PVRIg inhibitory domain (SEQ ID NO:151). In some embodiments, the iCAR comprises an AA2AR inhibitory domain (SEQ ID NO:152). In some embodiments, the iCAR comprises a signal peptide upstream of the iCAR portion, wherein the signal peptide is a CD8 alpha signal peptide (SEQ ID NO: 161), a GM-CSF signal peptide (SEQ ID NO: 162), or a mIgK signal peptide (SEQ ID NO: 306).

In some embodiments, the scFv has the VL and VH sequences of SN66E3.3 (SEQ ID NOs: 283 and 284). In some embodiments, the orientation of the iCAR VH and VL regions is VH-VL. In some embodiments, the orientation of the iCAR VH and VL regions is VL-VH. In some embodiments, the iCAR scFv comprises a (G4S)X3 linker (SEQ ID NO:81) linker that covalently connects the VH and the VL to form the iCAR scFv. In some embodiments, the iCAR scFv comprises a Whitlow linker (SEQ ID NO:82) linker that covalently connects the VH and the VL to form the iCAR scFv. In some embodiments, the iCAR comprises a CD8 alpha hinge domain (SEQ ID NO:84). In some embodiments, the iCAR comprises a CD28 hinge domain (SEQ ID NO:85). In some embodiments, the iCAR comprises a PD-1 hinge domain (SEQ ID NO:86). In some embodiments, the iCAR comprises a LIR1 Ig3-4 hinge domain (SEQ ID NO:87). In some embodiments, the iCAR comprises a LIR1 Ig-4 hinge domain (SEQ ID NO:88). In some embodiments, the iCAR comprises a LIR1 52 aa hinge domain (SEQ ID NO:89). In some embodiments, the iCAR comprises a LIR1 36 aa hinge domain (SEQ ID NO:90). In some embodiments, the iCAR comprises a LIR1 30 aa hinge domain (SEQ ID NO:91). In some embodiments, the iCAR comprises a LIR1 8 aa hinge domain (SEQ ID NO:92). In some embodiments, the iCAR comprises a CD33 hinge domain (SEQ ID NO:93). In some embodiments, the iCAR comprises a KIR2DL1 hinge domain (SEQ ID NO:94). In some embodiments, the iCAR comprises a LIR1 26 aa hinge domain (SEQ ID NO: 289). In some embodiments, the iCAR comprises PD-1 (47) hinge domain (SEQ ID NO: 290). In some embodiments, the iCAR comprises PD-1 (42) hinge domain (SEQ ID NO: 291). In some embodiments, the iCAR comprises PD-1 (36) hinge domain (SEQ ID NO: 292). In some embodiments, the iCAR comprises PD-1 (30) hinge domain (SEQ ID NO: 293). In some embodiments, the iCAR comprises PD-1 (26) hinge domain (SEQ ID NO: 294). In some embodiments, the iCAR comprises PD-1 (20) hinge domain (SEQ ID NO: 295). In some embodiments, the iCAR comprises a CD8 alpha transmembrane domain (SEQ ID NO:95). In some embodiments, the iCAR comprises a CD28 transmembrane domain (SEQ ID NO:96). In some embodiments, the iCAR comprises a PD-1 transmembrane domain (SEQ ID NO:97). In some embodiments, the iCAR comprises a LIR1 transmembrane domain (SEQ ID NO:98). In some embodiments, the iCAR comprises a CD33 transmembrane domain (SEQ ID NO:99). In some embodiments, the iCAR comprises a KIR2DL1 transmembrane domain (SEQ ID NO:100). In some embodiments, the iCAR comprises a PD-1 inhibitory domain (SEQ ID NO:101). In some embodiments, the iCAR comprises a KIR2DL1 inhibitory domain (SEQ ID NO:102). In some embodiments, the iCAR comprises a KIR2DL2 inhibitory domain (SEQ ID NO:103). In some embodiments, the iCAR comprises a KIR2DL3 inhibitory domain (SEQ ID NO:104). In some embodiments, the iCAR comprises a KIR2DL4 inhibitory domain (SEQ ID NO:105). In some embodiments, the iCAR comprises a KIR2DL5A inhibitory domain (SEQ ID NO:106). In some embodiments, the iCAR comprises a KIR3DL1 inhibitory domain (SEQ ID NO:107). In some embodiments, the iCAR comprises a KIR3DL2 inhibitory domain (SEQ ID NO:108). In some embodiments, the iCAR comprises a KIR3DL3 inhibitory domain (SEQ ID NO:109). In some embodiments, the iCAR comprises a LAIR1 inhibitory domain (SEQ ID NO:110). In some embodiments, the iCAR comprises a CD22 inhibitory domain (SEQ ID NO:111). In some embodiments, the iCAR comprises a CD33 inhibitory domain (SEQ ID NO:112). In some embodiments, the iCAR comprises a SIGLEC5 inhibitory domain (SEQ ID NO:113). In some embodiments, the iCAR comprises a SIGLEC6 inhibitory domain (SEQ ID NO:114). In some embodiments, the iCAR comprises a SIGLEC7 inhibitory domain (SEQ ID NO:115). In some embodiments, the iCAR comprises a SIGLEC8 inhibitory domain (SEQ ID NO:116). In some embodiments, the iCAR comprises a SIGLEC9 inhibitory domain (SEQ ID NO:117). In some embodiments, the iCAR comprises a SIGLEC10 inhibitory domain (SEQ ID NO:118). In some embodiments, the iCAR comprises a SIGLEC11 inhibitory domain (SEQ ID NO:119). In some embodiments, the iCAR comprises a SIGLEC12 inhibitory domain (SEQ ID NO:120). In some embodiments, the iCAR comprises a PECAM1/CD31 inhibitory domain (SEQ ID NO:121). In some embodiments, the iCAR comprises a CD200R1 inhibitory domain (SEQ ID NO:122). In some embodiments, the iCAR comprises a FCRL1 inhibitory domain (SEQ ID NO:123). In some embodiments, the iCAR comprises a FCRL2 inhibitory domain (SEQ ID NO:124). In some embodiments, the iCAR comprises a FCRL3 inhibitory domain (SEQ ID NO:125). In some embodiments, the iCAR comprises a FCRL4 inhibitory domain (SEQ ID NO:126). In some embodiments, the iCAR comprises a FCRL5 inhibitory domain (SEQ ID NO:127). In some embodiments, the iCAR comprises a SLAMF1 inhibitory domain (SEQ ID NO:128). In some embodiments, the iCAR comprises a SLAMF5 inhibitory domain (SEQ ID NO:129). In some embodiments, the iCAR comprises a BTLA inhibitory domain (SEQ ID NO:130). In some embodiments, the iCAR comprises a LAG3 inhibitory domain (SEQ ID NO:131). In some embodiments, the iCAR comprises a 2B4 inhibitory domain (SEQ ID NO:132). In some embodiments, the iCAR comprises a CD160 inhibitory domain (SEQ ID NO:133). In some embodiments, the iCAR comprises a CEACAM1 inhibitory domain (SEQ ID NO:134). In some embodiments, the iCAR comprises a TIM3 inhibitory domain (SEQ ID NO:135). In some embodiments, the iCAR comprises a VISTA inhibitory domain (SEQ ID NO:136). In some embodiments, the iCAR comprises a TIGIT inhibitory domain (SEQ ID NO:137). In some embodiments, the iCAR comprises a SIRPalpha inhibitory domain (SEQ ID NO:138). In some embodiments, the iCAR comprises a FcγRIIB inhibitory domain (SEQ ID NO:139). In some embodiments, the iCAR comprises a CD5 inhibitory domain (SEQ ID NO:140). In some embodiments, the iCAR comprises a CD300a inhibitory domain (SEQ ID NO:141). In some embodiments, the iCAR comprises a CD300f inhibitory domain (SEQ ID NO:142). In some embodiments, the iCAR comprises a LIR1 inhibitory domain (SEQ ID NO:143). In some embodiments, the iCAR comprises a LIR2 inhibitory domain (SEQ ID NO:144). In some embodiments, the iCAR comprises a LIR3 inhibitory domain (SEQ ID NO:145). In some embodiments, the iCAR comprises a LIR5 inhibitory domain (SEQ ID NO:146). In some embodiments, the iCAR comprises a LIR8 inhibitory domain (SEQ ID NO:147). In some embodiments, the iCAR comprises a Ly9 inhibitory domain (SEQ ID NO:148). In some embodiments, the iCAR comprises a 2×PD1(G4S) inhibitory domain (SEQ ID NO:149). In some embodiments, the iCAR comprises a 2×PD1(PD1) inhibitory domain (SEQ ID NO:150). In some embodiments, the iCAR comprises a PVRIg inhibitory domain (SEQ ID NO:151). In some embodiments, the iCAR comprises an AA2AR inhibitory domain (SEQ ID NO:152). In some embodiments, the WCAR comprises a signal peptide upstream of the iCAR portion, wherein the signal peptide is a CD8 alpha signal peptide (SEQ ID NO: 161), a GM-CSF signal peptide (SEQ ID NO: 162), or a mIgK signal peptide (SEQ ID NO: 306).

In some embodiments, the iCAR has a set of components shown in Tables 9-10 and/or an amino acid sequence shown in Tables 11-12.

TABLE 9
iCAR constructs

| | | VH SEQ | VL SEQ | Signal | scFv | | | |
|---|---|---|---|---|---|---|---|---|
| Construct | scFv | ID NO | ID NO | peptide | Linker | Hinge | TM | Signaling |
| | BB7.2 | 38 | 37 | CD8 alpha | (G4S) × 3 | PD-1 | PD-1 | PD-1 |
| MC0096 | 3PF12/C4 | 40 | 39 | CD8 alpha | (G4S) × 3 | PD-1 | PD-1 | PD-1 |
| (VR96) | | | | | | | | |
| MC0274 | 3PF12/F12 | 42 | 41 | CD8 alpha | (G4S) × 3 | PD-1 | PD-1 | PD-1 |
| (VR274) | | | | | | | | |
| MC0276 | 3PF12/B11 | 44 | 43 | CD8 alpha | (G4S) × 3 | PD-1 | PD-1 | PD-1 |
| (VR276) | | | | | | | | |
| MC0097 | W6/32 | 46 | 45 | CD8 alpha | (G4S) × 3 | PD-1 | PD-1 | PD-1 |
| (VR97) | | | | | | | | |
| MC0098 | BBM.1 | 48 | 47 | CD8 alpha | (G4S) × 3 | PD-1 | PD-1 | PD-1 |
| (VR98) | | | | | | | | |
| MC0099 | SN66E3.1 | 50 | 49 | CD8 alpha | (G4S) × 3 | PD-1 | PD-1 | PD-1 |
| (VR99) | | | | | | | | |
| MC0100 | Ha5C2.A2 | 52 | 51 | CD8 alpha | (G4S) × 3 | PD-1 | PD-1 | PD-1 |
| (VR100) | | | | | | | | |
| MC0101 | MWB1 | 54 | 53 | CD8 alpha | (G4S) × 3 | PD-1 | PD-1 | PD-1 |
| (VR101) | | | | | | | | |
| MC0102 | MWB1.1d | 56 | 55 | CD8 alpha | (G4S) × 3 | PD-1 | PD-1 | PD-1 |
| (VR102) | | | | | | | | |
| MC0372 | Hz.BB7.2 VH1-69_A18VK | 58 | 57 | CD8 alpha | (G4S) × 3 | CD8 alpha | CD8 alpha | 41BBz |
| (VR372) | | | | | | | | |
| MC0373 | Hz.BB7.2 VH1-69 (27, | 60 | 59 | CD8 alpha | (G4S) × 3 | CD8 alpha | CD8 alpha | 41BBz |
| (VR373) | 30)_A18 | | | | | | | |
| MC0374 | Hz.BB7.2 VH1-69 (27, | 62 | 61 | CD8 alpha | (G4S) × 3 | CD8 alpha | CD8 alpha | 41BBz |
| (VR374) | 30, 48)_A18 | | | | | | | |
| MC0375 | Hz.BB7.2 VH1-69 (27, | 64 | 63 | CD8 alpha | (G4S) × 3 | CD8 alpha | CD8 alpha | 41BBz |
| (VR375) | 30, 67)_A18 | | | | | | | |
| MC0376 | Hz.BB7.2 VH1-69 (27, | 66 | 65 | CD8 alpha | (G4S) × 3 | CD8 alpha | CD8 alpha | 41BBz |
| (VR376) | 30, 69)_A18 | | | | | | | |
| MC0377 | Hz.BB7.2 VH1-69 (27, | 68 | 67 | CD8 alpha | (G4S) × 3 | CD8 alpha | CD8 alpha | 41BBz |
| (VR377) | 30, 67, 69)_A18 | | | | | | | |
| MC0378 | Hz.BB7.2 VH1-3_A18 | 70 | 69 | CD8 alpha | (G4S) × 3 | CD8alpha | CD8 alpha | 41BBz |
| (VR378) | | | | | | | | |
| MC0379 | Hz.BB7.2 VH1-3(48)_A18 | 72 | 71 | CD8 alpha | (G4S) × 3 | CD8 alpha | CD8 alpha | 41BBz |
| (VR379) | | | | | | | | |
| MC0380 | Hz.BB7.2 VH1-3(67)_A18 | 74 | 73 | CD8 alpha | (G4S) × 3 | CD8 alpha | CD8 alpha | 41BBz |
| (VR380) | | | | | | | | |
| MC0381 | Hz.BB7.2 VH1-3(69)_A18 | 76 | 75 | CD8 alpha | (G4S) × 3 | CD8 alpha | CD8 alpha | 41BBz |
| (VR381) | | | | | | | | |
| MC0382 | Hz.BB7.2 VH1-3(71)_A18 | 78 | 77 | CD8 alpha | (G4S) × 3 | CD8 alpha | CD8 alpha | 41BBz |
| (VR382) | | | | | | | | |
| MC0383 | Hz.BB7.2 VH1-3(73)_A18 | 80 | 79 | CD8 alpha | (G4S) × 3 | CD8 alpha | CD8 alpha | 41BBz |
| (VR383) | | | | | | | | |
| MC0384 | 3PF12_274_LIR1_HER2_shRNA(A2) | 40 | 41 | CD8 alpha | 3PF12_274 | (G4S) × 3 | PD-1 | LIR-1 |
| (VR384) | | | | | | | | |
| MC0385 | 3PF12_276_LIR1_HER2_shRNA(A2) | 44 | 43 | CD8 alpha | 3PF12_276 | (G4S) × 3 | PD-1 | LIR-1 |
| (VR385) | | | | | | | | |
| MC0386 | MWB1.1_HL_LIR1_HER2_shRNA(A2) | 56 | 55 | CD8 alpha | MWB1.1_HL | (G4S) × 3 | PD-1 | LIR-1 |
| (VR386) | | | | | | | | |
| MC0387 | MWB1.1_LH_LIR1_HER2_shRNA(A2) | 56 | 55 | CD8 alpha | MWB1.1_LH | (G4S) × 3 | PD-1 | LIR-1 |
| (VR387) | | | | | | | | |
| MC0388 | MWB1.2_HL_LIR1_HER2_shRNA(A2) | 164 | 163 | CD8 alpha | MWB1.2_HL | (G4S) × 3 | PD-1 | LIR-1 |
| (VR388) | | | | | | | | |
| MC0389 | MWB1.2_LH_LIR1_HER2_shRNA(A2) | 164 | 163 | CD8 alpha | MWB1.2_LH | (G4S) × 3 | PD-1 | LIR-1 |
| (VR389) | | | | | | | | |
| MC0390 | SN66E3.1_HL_LIR1_HER2_shRNA(A2) | 50 | 49 | CD8 alpha | SN66E3.1_HL | (G4S) × 3 | PD-1 | LIR-1 |
| (VR390) | | | | | | | | |
| MC0391 | SN66E3.1_LH_LIR1_HER2_shRNA(A2) | 50 | 49 | CD8 alpha | SN66E3.1_LH | (G4S) × 3 | PD-1 | LIR-1 |
| (VR391) | | | | | | | | |
| MC0446 | SN66E3.2_HL_LIR1_HER2_{—} | 166 | 165 | CD8 alpha | SN66E3.2_HL | (G4S) × 3 | LIR1 | LIR-1 |
| (VR446) | | | | | | | | |
| MC0447 | SN66E3.2_LH_LIR1_HER2_{—} | 166 | 165 | CD8 alpha | SN66E3.2_LH | (G4S) × 3 | LIR1 | LIR-1 |
| (VR447) | | | | | | | | |
| MC0448 | SN66E3.3(HL)_LIR1(26)_HER2 | 284 | 283 | CD8 alpha | SN66E.3.3_HL | (G4S) × 3 | LIR1 | LIR1 |
| (VR448) | | | | | | or none | | |
| MC449 | SN66E3.3(LH)_LIR1(26)_HER2 | 284 | 283 | CD8 alpha | SN66E3.3_LH | (G4S) × 3 | LIR1 | LIR1 |
| (VR449) | | | | | | | | |
| MC0428 | HzBB7.2.1_H69_LIR1_H | 64 | 63 | CD8 alpha | HzBB7.2_H69 | (G4S) × 3 | LIR1 | LIR-1 |
| (VR428) | | | | | | | | |
| MC0421 | HzBB7.2.2_H3_LIR1_) | 72 | 71 | CD8 alpha | HzBB7.2_H3 | (G4S) × 3 | LIR1 | LIR-1 |
| (VR421) | | | | | | | | |

TABLE 10
iCAR constructs

| | Construct | Signal | | scFv | | | |
|---|---|---|---|---|---|---|---|
| Construct | Name | Peptide | scFv | Linker | Hinge | TM | Signaling |
| MC0058 | 1 × ITIM | CD8alpha | BB7.2 | (G4S) × 3 | PD-1 | PD-1 | 1 × ITIM |
| (VR58) | | | VH VL | | | | PD-1 |
| MC0059 | 2 × ITIM | CD8alpha | BB7.2 | (G4S) × 3 | PD-1 | PD-1 | 2 × ITIM |
| (VR59) | | | VH VL | | | | PD-1 |
| MC0060 | 3 × ITIM | CD8alpha | BB7.2 | (G4S) × 3 | PD-1 | PD-1 | 3 × ITIM |
| (VR60) | | | VH VL | | | | PD-1 |
| MC0061 | 4 × ITIM | CD8alpha | BB7.2 | (G4S) × 3 | PD-1 | PD-1 | 4 × ITIM |
| (VR61) | | | VH VL | | | | PD-1 |
| MC0062 | 5 × ITIM | CD8alpha | BB7.2 | (G4S) × 3 | PD-1 | PD-1 | 5 × ITIM |
| (VR62) | | | VH VL | | | | PD-1 |
| MC0063 | 1 × ITSM | CD8alpha | BB7.2 | (G4S) × 3 | PD-1 | PD-1 | 1 × ITSM |
| (VR63) | | | VH VL | | | | PD-1 |
| MC0064 | 2 × ITSM | CD8alpha | BB7.2 | (G4S) × 3 | PD-1 | PD-1 | 2 × ITSM |
| (VR64) | | | VH VL | | | | PD-1 |
| MC0065 | 3 × ITSM | CD8alpha | BB7.2 | (G4S) × 3 | PD-1 | PD-1 | 3 × ITSM |
| (VR65) | | | VH VL | | | | PD-1 |
| MC0066 | 4 × ITSM | CD8alpha | BB7.2 | (G4S) × 3 | PD-1 | PD-1 | 4 × ITSM |
| (VR66) | | | VH VL | | | | PD-1 |
| MC0067 | 5 × ITSM | CD8alpha | BB7.2 | (G4S) × 3 | PD-1 | PD-1 | 5 × ITSM |
| (VR67) | | | VH VL | | | | PD-1 |
| MC0068 | 2 × PD1(G4S) | CD8alpha | BB7.2 | (G4S) × 3 | PD-1 | PD-1 | 2 × PD-1 |
| (VR68) | | | VH VL | | | | (G4S) × 2 |
| MC0069 | 2 × PD1(PD1) | CD8alpha | BB7.2 | (G4S) × 3 | PD-1 | PD-1 | 2 × PD-1 |
| (VR69) | | | VH VL | | | | (PD1 linker) |

TABLE 11
iCAR constructs

| | | SEQ | |
|---|---|---|---|
| | | ID | |
| Construct | scFv | NO | Amino acid sequence |
| MC0387 | MWB1.1_ | 255 | MALPVTALLLPLALLLHAARPQSALTQPPSASG |
| (VR387) | LH_LIR1_ | | SPGQSVTISCTGTSSDVGGYKYVSWYQHHPDK |
| | HER2_ | | APKLMIYEVNKRPSGVPDRFSGSKSDNTASLTV |
| | shRNA(A2) | | SGLQAEDEADYYCSSYAGSNNWVFGGGTKLTV |
| | | | LGGGGSGGGGSGGGGSQVQLVESGGGVVQPG |
| | | | GSLRLSCAASGFTFSTYGMHWVRQAPGKGLEW |
| | | | VASISYDGSNKYYADSGQGRFTISRDTSKNSLYL |
| | | | QMNSLRAEDTAVYYCAIGIYGAYSFDYWGQGT |
| | | | LVTVSSTERRAEVPTAHPSPSPRPAGQFQTLVVG |
| | | | VVGGLLGSLVLLVWVLAVILRHRRQGKHWTST |
| | | | QRKADFQHPAGAVGPEPTDRGLQWRSSPAADA |
| | | | QEENLYAAVKHTQPEDGVEMDTRSPHDEDPQA |
| | | | VTYAEVKHSRPRREMASPPSPLSGEFLDTKDRQ |
| | | | AEEDRQMDTEAAASEAPQDVTYAQLHSLTLRR |
| | | | EATEPPPSQEGPSPAVPSIYATLAIH |
| MC0389 | MWB1.2_ | 256 | MALPVTALLLPLALLLHAARPQSALTQPPSASG |
| (VR389) | LH_LIR1_ | | SPGQSVTISCTGTSSDVGGYKYVSWYQQHPGK |
| | HER2_ | | APKLMIYEVNKRPSGVPDRFSGSKSGNTASLTV |
| | shRNA(A2) | | SGLQAEDEADYYCSSYAGSNNWVFGGGTKLTV |
| | | | LGGGGSGGGGSGGGGSQVQLVESGGGVVQPG |
| | | | GSLRLSCAASGFTFSTYGMHWVRQAPGKGLEW |
| | | | VASISYDGSNKYYADSGQGRFTISRDTSKNSLYL |
| | | | QMNSLRAEDTAVYYCAIGIYGAYSFDYWGQGT |
| | | | LVTVSSTERRAEVPTAHPSPSPRPAGQFQTLVVG |
| | | | VVGGLLGSLVLLVWVLAVILRHRRQGKHWTST |
| | | | QRKADFQHPAGAVGPEPTDRGLQWRSSPAADA |
| | | | QEENLYAAVKHTQPEDGVEMDTRSPHDEDPQA |
| | | | VTYAEVKHSRPRREMASPPSPLSGEFLDTKDRQ |
| | | | AEEDRQMDTEAAASEAPQDVTYAQLHSLTLRR |
| | | | EATEPPPSQEGPSPAVPSIYATLAIH |
| MC0391 | SN66E3.1_ | 257 | MALPVTALLLPLALLLHAARPDIVMTQSPDSLA |
| (VR391) | LH_LIR1_ | | VSLGERATISCKSSQSVLYSSNNKNYLAWYQQK |
| | HER2_ | | LGQPPKLLIYWASTRESGVPDRFSGSGSGTNFTL |
| | shRNA(A2) | | TISSLQAENVAVYYCQQYYGTPFTFGGGTKVEI |
| | | | KGGGGSGGGGSGGGGSQVQLVQSGAEVKKPG |
| | | | ASVKVSCKASGYTFTDYYLHWVRQAPGQGLE |
| | | | WMGWINPYTGGTNYAQKFQGRVTMTRDASIST |
| | | | VYMELSGLTSDDTAVHFCARAGASYYDFWSG |
| | | | WVFDYWGQGTLVTVSSTERRAEVPTAHPSPSPR |
| | | | PAGQFQTLVVGVVGGLLGSLVLLVWVLAVILR |
| | | | HRRQGKHWTSTQRKADFQHPAGAVGPEPTDRG |
| | | | LQWRSSPAADAQEENLYAAVKHTQPEDGVEM |
| | | | DTRSPHDEDPQAVTYAEVKHSRPRREMASPPSP |
| | | | LSGEFLDTKDRQAEEDRQMDTEAAASEAPQDV |
| | | | TYAQLHSLTLRREATEPPPSQEGPSPAVPSIYATL |
| | | | AIH |
| MC0447 | SN66E3.2 | 258 | MALPVTALLLPLALLLHAARPDIVMTQSPDSLA |
| (VR447) | (LH)_LIR1 | | VSLGERATISCKSSQSVLYSSNNKNYLAWYQQK |
| | (30)_HER2 | | PGQPPKLLIYWASTRESGVPDRFSGSGSGTDFTL |
| | | | TISSLQAEDVAVYYCQQYYGTPFTFGGGTKVEI |
| | | | KGGGGSGGGGSGGGGSQVQLVQSGAEVKKPG |
| | | | ASVKVSCKASGYTFTDYYLHWVRQAPGQGLE |
| | | | WMGWINPYTGGTNYAQKFQGRVTMTRDTSIST |
| | | | AYMELSGLTSDDTAVYYCARAGASYYDFWSG |
| | | | WVFDYWGQGTLVTVSSGPTSTSGPEDQPLTPTG |
| | | | SDPQSGLGRHLGVVIGILVAVILLLLLLLLLFLIL |
| | | | RHRRQGKHWTSTQRKADFQHPAGAVGPEPTDR |
| | | | GLQWRSSPAADAQEENLYAAVKHTQPEDGVE |
| | | | MDTRSPHDEDPQAVTYAEVKHSRPRREMASPPS |
| | | | PLSGEFLDTKDRQAEEDRQMDTEAAASEAPQD |
| | | | VTYAQLHSLTLRREATEPPPSQEGPSPAVPSIYA |
| | | | TLAIH |
| MC0449 | SN66E3.3 | 305 | MALPVTALLLPLALLLHAARPDIVMTQSPDSLA |
| (VR449) | (LH)_LIR1 | | VSLGERATISCKSSQSVLYSSNNKNYLAWYQQK |
| | (26)_HER2 | | PGQPPKLLIYWASTRESGVPDRFSGSGSGTDFTL |
| | | | TISSLQAEDVAVYYCQQYYGTPFTFGGGTKVEI |
| | | | KGGGGSGGGGSGGGGSQVQLVQSGAEVKKPG |
| | | | ASVKVSCKASGYTFTDYYLHWVRQAPGQGLE |
| | | | WMGWINPYTGGTNYAQKFQGRVTMTRDTSIST |
| | | | AYMELSRLRSEDTAVYYCARAGASYYDFWSG |
| | | | WVFDYWGQGTLVTVSSTSGPEDQPLTPTGSDPQ |
| | | | SGLGRHLGVVIGILVAVILLLLLLLLLFLILRHRR |
| | | | QGKHWTSTQRKADFQHPAGAVGPEPTDRGLQ |
| | | | WRSSPAADAQEENLYAAVKHTQPEDGVEMDT |
| | | | RSPHDEDPQAVTYAEVKHSRPRREMASPPSPLS |
| | | | GEFLDTKDRQAEEDRQMDTEAAASEAPQDVTY |
| | | | AQLHSLTLRREATEPPPSQEGPSPAVPSIYATLAI |
| | | | H |
| MC0428 | HzBB7.2.1_ | 259 | MALPVTALLLPLALLLHAARPQVQLVQSGAEV |
| (VR428) | _LIR1 | | KKPGSSVKVSCKASGYTFTSYHIQWVRQAPGQ |
| | (52)_HER2 | | GLEWMGWIYPGDGSTQYNEKFKGRTTITADKS |
| | | | TSTAYMELSSLRSEDTAVYYCAREGTYYAMDY |
| | | | WGQGTLVTVSSGGGGSGGGGSGGGGSDVVMT |
| | | | QTPLSLSVTPGQPASISCRSSQSIVHSNGNTYLE |
| | | | WYLQKPGQSPQLLIYKVSNRFSGVPDRFSGSGS |
| | | | GTDFTLKISRVEAEDVGVYYCFQGSHVPRTFGG |
| | | | GTKVEIKHPSDPLELVVSGPSGGPSSPTTGPTSTS |
| | | | GPEDQPLTPTGSDPQSGLGRHLGVVIGILVAVIL |
| | | | LLLLLLLLFLILRHRRQGKHWTSTQRKADFQHP |
| | | | AGAVGPEPTDRGLQWRSSPAADAQEENLYAAV |
| | | | KHTQPEDGVEMDTRSPHDEDPQAVTYAEVKHS |
| | | | RPRREMASPPSPLSGEFLDTKDRQAEEDRQMDT |
| | | | EAAASEAPQDVTYAQLHSLTLRREATEPPPSQE |
| | | | GPSPAVPSIYATLAIH |
| MC0421 | HzBB7.2.2_ | 260 | MALPVTALLLPLALLLHAARPQVQLVQSGAEV |
| (VR421) | H3_LIR1_ | | KKPGASVKVSCKASGYTFTSYHIQWVRQAPGQ |
| | HER2_ | | RLEWIGWIYPGDGSTQYNEKFKGRVTITRDTSA |
| | | | STAYMELSSLRSEDTAVYYCAREGTYYAMDY |
| | | | WGQGTLVTVSSGGGGSGGGGSGGGGSDVVMT |
| | | | QTPLSLSVTPGQPASISCRSSQSIVHSNGNTYLE |
| | | | WYLQKPGQSPQLLIYKVSNRFSGVPDRFSGSGS |
| | | | GTDFTLKISRVEAEDVGVYYCFQGSHVPRTFGG |
| | | | GTKVEIKHPSDPLELVVSGPSGGPSSPTTGPTSTS |
| | | | GPEDQPLTPTGSDPQSGLGRHLGVVIGILVAVIL |
| | | | LLLLLLLLFLILRHRRQGKHWTSTQRKADFQHP |
| | | | AGAVGPEPTDRGLQWRSSPAADAQEENLYAAV |
| | | | KHTQPEDGVEMDTRSPHDEDPQAVTYAEVKHS |
| | | | RPRREMASPPSPLSGEFLDTKDRQAEEDRQMDT |
| | | | EAAASEAPQDVTYAQLHSLTLRREATEPPPSQE |
| | | | GPSPAVPSIYATLAIH |

TABLE 12
iCAR constructs

| | | SEQ | |
|---|---|---|---|
| | Construct | ID | |
| Construct | Name | NO | Full length iCAR sequence |
| MC0058 | 1xITIM | 261 | MALPVTALLLPLALLLHAARPEQKLISEEDLQV |
| (VR58) | | | QLQQSGPELVKPGASVKMSCKASGYTFTSYHIQ |
| | | | WVKQRPGQGLEWIGWIYPGDGSTQYNEKFKGK |
| | | | TTLTADKSSSTAYMLLSSLTSEDSAIYFCAREGT |
| | | | YYAMDYWGQGTSVTVSSGGGGSGGGGSGGGG |
| | | | SDVLMTQTPLSLPVSLGDQVSISCRSSQSIVHSN |
| | | | GNTYLEWYLQKPGQSPKLLIYKVSNRFSGVPDR |
| | | | FSGSGSGTDFTLKISRVEAEDLGVYYCFQGSHVP |
| | | | RTFGGGTKLEIKTERRAEVPTAHPSPSPRPAGQF |
| | | | QTLVVGVVGGLLGSLVLLVWVLAVICSRAARG |
| | | | TIGARRTGQPLKEDPSAVPVFSVDYGELVFPSG |
| | | | MGTSSPARRGSADGPRSAQPLRPEDGHCSWPL |
| MC0059 | 2xITIM | 262 | MALPVTALLLPLALLLHAARPEQKLISEEDLQV |
| (VR59) | | | QLQQSGPELVKPGASVKMSCKASGYTFTSYHIQ |
| | | | WVKQRPGQGLEWIGWIYPGDGSTQYNEKFKGK |
| | | | TTLTADKSSSTAYMLLSSLTSEDSAIYFCAREGT |
| | | | YYAMDYWGQGTSVTVSSGGGGSGGGGSGGGG |
| | | | SDVLMTQTPLSLPVSLGDQVSISCRSSQSIVHSN |
| | | | GNTYLEWYLQKPGQSPKLLIYKVSNRFSGVPDR |
| | | | FSGSGSGTDFTLKISRVEAEDLGVYYCFQGSHVP |
| | | | RTFGGGTKLEIKTERRAEVPTAHPSPSPRPAGQF |
| | | | QTLVVGVVGGLLGSLVLLVWVLAVICSRAARG |
| | | | TIGARRTGQPLKEDPSAVPVFSVDYGELDFQWR |
| | | | EKTPEPPVPCVPEQVDYGELVFPSGMGTSSPAR |
| | | | RGSADGPRSAQPLRPEDGHCSWPL |
| MC0060 | 3xITIM | 263 | MALPVTALLLPLALLLHAARPEQKLISEEDLQV |
| (VR60) | | | QLQQSGPELVKPGASVKMSCKASGYTFTSYHIQ |
| | | | WVKQRPGQGLEWIGWIYPGDGSTQYNEKFKGK |
| | | | TTLTADKSSSTAYMLLSSLTSEDSAIYFCAREGT |
| | | | YYAMDYWGQGTSVTVSSGGGGSGGGGSGGGG |
| | | | SDVLMTQTPLSLPVSLGDQVSISCRSSQSIVHSN |
| | | | GNTYLEWYLQKPGQSPKLLIYKVSNRFSGVPDR |
| | | | FSGSGSGTDFTLKISRVEAEDLGVYYCFQGSHVP |
| | | | RTFGGGTKLEIKTERRAEVPTAHPSPSPRPAGQF |
| | | | QTLVVGVVGGLLGSLVLLVWVLAVICSRAARG |
| | | | TIGARRTGQPLKEDPSAVPVFSVDYGELDFQWR |
| | | | EKTPEPPVPCVPEQVDYGELDFQWREKTPEPPV |
| | | | PCVPEQVDYGELVFPSGMGTSSPARRGSADGPR |
| | | | SAQPLRPEDGHCSWPL |
| MC0061 | 4xITIM | 264 | MALPVTALLLPLALLLHAARPEQKLISEEDLQV |
| (VR61) | | | QLQQSGPELVKPGASVKMSCKASGYTFTSYHIQ |
| | | | WVKQRPGQGLEWIGWIYPGDGSTQYNEKFKGK |
| | | | TTLTADKSSSTAYMLLSSLTSEDSAIYFCAREGT |
| | | | YYAMDYWGQGTSVTVSSGGGGSGGGGSGGGG |
| | | | SDVLMTQTPLSLPVSLGDQVSISCRSSQSIVHSN |
| | | | GNTYLEWYLQKPGQSPKLLIYKVSNRFSGVPDR |
| | | | FSGSGSGTDFTLKISRVEAEDLGVYYCFQGSHVP |
| | | | RTFGGGTKLEIKTERRAEVPTAHPSPSPRPAGQF |
| | | | QTLVVGVVGGLLGSLVLLVWVLAVICSRAARG |
| | | | TIGARRTGQPLKEDPSAVPVFSVDYGELDFQWR |
| | | | EKTPEPPVPCVPEQVDYGELDFQWREKTPEPPV |
| | | | PCVPEQVDYGELDFQWREKTPEPPVPCVPEQVD |
| | | | YGELVFPSGMGTSSPARRGSADGPRSAQPLRPE |
| | | | DGHCSWPL |
| MC0062 | 5xITIM | 265 | MALPVTALLLPLALLLHAARPEQKLISEEDLQV |
| (VR62) | | | QLQQSGPELVKPGASVKMSCKASGYTFTSYHIQ |
| | | | WVKQRPGQGLEWIGWIYPGDGSTQYNEKFKGK |
| | | | TTLTADKSSSTAYMLLSSLTSEDSAIYFCAREGT |
| | | | YYAMDYWGQGTSVTVSSGGGGSGGGGSGGGG |
| | | | SDVLMTQTPLSLPVSLGDQVSISCRSSQSIVHSN |
| | | | GNTYLEWYLQKPGQSPKLLIYKVSNRFSGVPDR |
| | | | FSGSGSGTDFTLKISRVEAEDLGVYYCFQGSHVP |
| | | | RTFGGGTKLEIKTERRAEVPTAHPSPSPRPAGQF |
| | | | QTLVVGVVGGLLGSLVLLVWVLAVICSRAARG |
| | | | TIGARRTGQPLKEDPSAVPVFSVDYGELDFQWR |
| | | | EKTPEPPVPCVPEQVDYGELDFQWREKTPEPPV |
| | | | PCVPEQVDYGELDFQWREKTPEPPVPCVPEQVD |
| | | | YGELDFQWREKTPEPPVPCVPEQVDYGELVFPS |
| | | | GMGTSSPARRGSADGPRSAQPLRPEDGHCSWPL |
| MC0063 | 1×ITSM | 266 | MALPVTALLLPLALLLHAARPEQKLISEEDLQV |
| (VR63) | | | QLQQSGPELVKPGASVKMSCKASGYTFTSYHIQ |
| | | | WVKQRPGQGLEWIGWIYPGDGSTQYNEKFKGK |
| | | | TTLTADKSSSTAYMLLSSLTSEDSAIYFCAREGT |
| | | | YYAMDYWGQGTSVTVSSGGGGSGGGGSGGGG |
| | | | SDVLMTQTPLSLPVSLGDQVSISCRSSQSIVHSN |
| | | | GNTYLEWYLQKPGQSPKLLIYKVSNRFSGVPDR |
| | | | FSGSGSGTDFTLKISRVEAEDLGVYYCFQGSHVP |
| | | | RTFGGGTKLEIKTERRAEVPTAHPSPSPRPAGQF |
| | | | QTLVVGVVGGLLGSLVLLVWVLAVICSRAARG |
| | | | TIGARRTGQPLKEDPSAVPVFSTEYATIVFPSGM |
| | | | GTSSPARRGSADGPRSAQPLRPEDGHCSWPL |
| MC0064 | 2xITSM | 267 | MALPVTALLLPLALLLHAARPEQKLISEEDLQV |
| (VR64) | | | QLQQSGPELVKPGASVKMSCKASGYTFTSYHIQ |
| | | | WVKQRPGQGLEWIGWIYPGDGSTQYNEKFKGK |
| | | | TTLTADKSSSTAYMLLSSLTSEDSAIYFCAREGT |
| | | | YYAMDYWGQGTSVTVSSGGGGSGGGGSGGGG |
| | | | SDVLMTQTPLSLPVSLGDQVSISCRSSQSIVHSN |
| | | | GNTYLEWYLQKPGQSPKLLIYKVSNRFSGVPDR |
| | | | FSGSGSGTDFTLKISRVEAEDLGVYYCFQGSHVP |
| | | | RTFGGGTKLEIKTERRAEVPTAHPSPSPRPAGQF |
| | | | QTLVVGVVGGLLGSLVLLVWVLAVICSRAARG |
| | | | TIGARRTGQPLKEDPSAVPVESTEYATIDFQWRE |
| | | | KTPEPPVPCVPEQTEYATIVFPSGMGTSSPARRG |
| | | | SADGPRSAQPLRPEDGHCSWPL |
| MC0065 | 3xITSM | 268 | MALPVTALLLPLALLLHAARPEQKLISEEDLQV |
| (VR65) | | | QLQQSGPELVKPGASVKMSCKASGYTFTSYHIQ |
| | | | WVKQRPGQGLEWIGWIYPGDGSTQYNEKFKGK |
| | | | TTLTADKSSSTAYMLLSSLTSEDSAIYFCAREGT |
| | | | YYAMDYWGQGTSVTVSSGGGGSGGGGSGGGG |
| | | | SDVLMTQTPLSLPVSLGDQVSISCRSSQSIVHSN |
| | | | GNTYLEWYLQKPGQSPKLLIYKVSNRFSGVPDR |
| | | | FSGSGSGTDFTLKISRVEAEDLGVYYCFQGSHVP |
| | | | RTFGGGTKLEIKTERRAEVPTAHPSPSPRPAGQF |
| | | | QTLVVGVVGGLLGSLVLLVWVLAVICSRAARG |
| | | | TIGARRTGQPLKEDPSAVPVESTEYATIDFQWRE |
| | | | KTPEPPVPCVPEQTEYATIDFQWREKTPEPPVPC |
| | | | VPEQTEYATIVFPSGMGTSSPARRGSADGPRSA |
| | | | QPLRPEDGHCSWPL |
| MC0066 | 4xITSM | 269 | MALPVTALLLPLALLLHAARPEQKLISEEDLQV |
| (VR66) | | | QLQQSGPELVKPGASVKMSCKASGYTFTSYHIQ |
| | | | WVKQRPGQGLEWIGWIYPGDGSTQYNEKFKGK |
| | | | TTLTADKSSSTAYMLLSSLTSEDSAIYFCAREGT |
| | | | YYAMDYWGQGTSVTVSSGGGGSGGGGSGGGG |
| | | | SDVLMTQTPLSLPVSLGDQVSISCRSSQSIVHSN |
| | | | GNTYLEWYLQKPGQSPKLLIYKVSNRFSGVPDR |
| | | | FSGSGSGTDFTLKISRVEAEDLGVYYCFQGSHVP |
| | | | RTFGGGTKLEIKTERRAEVPTAHPSPSPRPAGQF |
| | | | QTLVVGVVGGLLGSLVLLVWVLAVICSRAARG |
| | | | TIGARRTGQPLKEDPSAVPVFSTEYATIDFQWRE |
| | | | KTPEPPVPCVPEQTEYATIDFQWREKTPEPPVPC |
| | | | VPEQTEYATIDFQWREKTPEPPVPCVPEQTEYAT |
| | | | IVFPSGMGTSSPARRGSADGPRSAQPLRPEDGHC |
| | | | SWPL |
| MC0067 | 5xITSM | 270 | MALPVTALLLPLALLLHAARPEQKLISEEDLQV |
| (VR67) | | | QLQQSGPELVKPGASVKMSCKASGYTFTSYHIQ |
| | | | WVKQRPGQGLEWIGWIYPGDGSTQYNEKFKGK |
| | | | TTLTADKSSSTAYMLLSSLTSEDSAIYFCAREGT |
| | | | YYAMDYWGQGTSVTVSSGGGGSGGGGSGGGG |
| | | | SDVLMTQTPLSLPVSLGDQVSISCRSSQSIVHSN |
| | | | GNTYLEWYLQKPGQSPKLLIYKVSNRFSGVPDR |
| | | | FSGSGSGTDFTLKISRVEAEDLGVYYCFQGSHVP |
| | | | RTFGGGTKLEIKTERRAEVPTAHPSPSPRPAGQF |
| | | | QTLVVGVVGGLLGSLVLLVWVLAVICSRAARG |
| | | | TIGARRTGQPLKEDPSAVPVESTEYATIDFQWRE |
| | | | KTPEPPVPCVPEQTEYATIDFQWREKTPEPPVPC |
| | | | VPEQTEYATIDFQWREKTPEPPVPCVPEQTEYAT |
| | | | IDFQWREKTPEPPVPCVPEQTEYATIVFPSGMGT |
| | | | SSPARRGSADGPRSAQPLRPEDGHCSWPL |
| MC0068 | 2xPD1 | 271 | MALPVTALLLPLALLLHAARPEQKLISEEDLQV |
| (VR68) | (G4S) | | QLQQSGPELVKPGASVKMSCKASGYTFTSYHIQ |
| | | | WVKQRPGQGLEWIGWIYPGDGSTQYNEKFKGK |
| | | | TTLTADKSSSTAYMLLSSLTSEDSAIYFCAREGT |
| | | | YYAMDYWGQGTSVTVSSGGGGSGGGGSGGGG |
| | | | SDVLMTQTPLSLPVSLGDQVSISCRSSQSIVHSN |
| | | | GNTYLEWYLQKPGQSPKLLIYKVSNRFSGVPDR |
| | | | FSGSGSGTDFTLKISRVEAEDLGVYYCFQGSHVP |
| | | | RTFGGGTKLEIKTERRAEVPTAHPSPSPRPAGQF |
| | | | QTLVVGVVGGLLGSLVLLVWVLAVICSRAARG |
| | | | TIGARRTGQPLKEDPSAVPVFSVDYGELDFQWR |
| | | | EKTPEPPVPCVPEQTEYATIVFPSGMGTSSPARR |
| | | | GSADGPRSAQPLRPEDGHCSWPLGGGGSGGGG |
| | | | SCSRAARGTIGARRTGQPLKEDPSAVPVFSVDY |
| | | | GELDFQWREKTPEPPVPCVPEQTEYATIVFPSG |
| | | | MGTSSPARRGSADGPRSAQPLRPEDGHCSWPL |
| MC0069 | 2xPD1 | 272 | MALPVTALLLPLALLLHAARPEQKLISEEDLQV |
| (VR69) | (PD1) | | QLQQSGPELVKPGASVKMSCKASGYTFTSYHIQ |
| | | | WVKQRPGQGLEWIGWIYPGDGSTQYNEKFKGK |
| | | | TTLTADKSSSTAYMLLSSLTSEDSAIYFCAREGT |
| | | | YYAMDYWGQGTSVTVSSGGGGSGGGGSGGGG |
| | | | SDVLMTQTPLSLPVSLGDQVSISCRSSQSIVHSN |
| | | | GNTYLEWYLQKPGQSPKLLIYKVSNRFSGVPDR |
| | | | FSGSGSGTDFTLKISRVEAEDLGVYYCFQGSHVP |
| | | | RTFGGGTKLEIKTERRAEVPTAHPSPSPRPAGQF |
| | | | QTLVVGVVGGLLGSLVLLVWVLAVICSRAARG |
| | | | TIGARRTGQPLKEDPSAVPVFSVDYGELDFQWR |
| | | | EKTPEPPVPCVPEQTEYATIDFQWREKTPEPPVP |
| | | | CVPEQVDYGELDFQWREKTPEPPVPCVPEQTEY |
| | | | ATIVFPSGMGTSSPARRGSADGPRSAQPLRPEDG |
| | | | HCSWPL |
| MC0456 | LIR1 | 327 | MALPVTALLLPLALLLHAARPQVQLVQSGAEV |
| (VR456) | (ITIM1)X4 | | KKPGASVKVSCKASGYTFTSYHIQWVRQAPGQ |
| | | | RLEWIGWIYPGDGSTQYNEKFKGRVTITRDTSA |
| | | | STAYMELSSLRSEDTAVYYCAREGTYYAMDY |
| | | | WGQGTLVTVSSGGGGSGGGGSGGGGSDVVMT |
| | | | QTPLSLSVTPGQPASISCRSSQSIVHSNGNTYLE |
| | | | WYLQKPGQSPQLLIYKVSNRFSGVPDRFSGSGS |
| | | | GTDFTLKISRVEAEDVGVYYCFQGSHVPRTFGG |
| | | | GTKVEIKTERRAEVPTAHPSPSPRPAGQFQTLVV |
| | | | GVVGGLLGSLVLLVWVLAVILRHRRQGKHWTS |
| | | | TQRKADFQHPAGAVGPEPTDRGLQWRSSPAAD |
| | | | AQEENLYAAVKHTQPEDGVEMDTRSPHDEDPQ |
| | | | ANLYAAVKHSRPRREMASPPSPLSGEFLDTKDR |
| | | | QAEEDRQMDTEAAASEAPQDNLYAAVHSLTLR |
| | | | REATEPPPSQEGPSPAVPNLYAAVAIH |
| MC0457 | LIR1 | 328 | MALPVTALLLPLALLLHAARPQVQLVQSGAEV |
| (VR457) | (ITIM2)X4 | | KKPGASVKVSCKASGYTFTSYHIQWVRQAPGQ |
| | | | RLEWIGWIYPGDGSTQYNEKFKGRVTITRDTSA |
| | | | STAYMELSSLRSEDTAVYYCAREGTYYAMDY |
| | | | WGQGTLVTVSSGGGGSGGGGSGGGGSDVVMT |
| | | | QTPLSLSVTPGQPASISCRSSQSIVHSNGNTYLE |
| | | | WYLQKPGQSPQLLIYKVSNRFSGVPDRFSGSGS |
| | | | GTDFTLKISRVEAEDVGVYYCFQGSHVPRTFGG |
| | | | GTKVEIKTERRAEVPTAHPSPSPRPAGQFQTLVV |
| | | | GVVGGLLGSLVLLVWVLAVILRHRRQGKHWTS |
| | | | TQRKADFQHPAGAVGPEPTDRGLQWRSSPAAD |
| | | | AQEEVTYAEVKHTQPEDGVEMDTRSPHDEDPQ |
| | | | AVTYAEVKHSRPRREMASPPSPLSGEFLDTKDR |
| | | | QAEEDRQMDTEAAASEAPQDVTYAEVHSLTLR |
| | | | REATEPPPSQEGPSPAVPVTYAEVAIH |
| MC0458 | LIR1 | 329 | MALPVTALLLPLALLLHAARPQVQLVQSGAEV |
| (VR458) | (ITIM3)X4 | | KKPGASVKVSCKASGYTFTSYHIQWVRQAPGQ |
| | | | RLEWIGWIYPGDGSTQYNEKFKGRVTITRDTSA |
| | | | STAYMELSSLRSEDTAVYYCAREGTYYAMDY |
| | | | WGQGTLVTVSSGGGGSGGGGSGGGGSDVVMT |
| | | | QTPLSLSVTPGQPASISCRSSQSIVHSNGNTYLE |
| | | | WYLQKPGQSPQLLIYKVSNRFSGVPDRFSGSGS |
| | | | GTDFTLKISRVEAEDVGVYYCFQGSHVPRTFGG |
| | | | GTKVEIKTERRAEVPTAHPSPSPRPAGQFQTLVV |
| | | | GVVGGLLGSLVLLVWVLAVILRHRRQGKHWTS |
| | | | TQRKADFQHPAGAVGPEPTDRGLQWRSSPAAD |
| | | | AQEEVTYAQLKHTQPEDGVEMDTRSPHDEDPQ |
| | | | AVTYAQLKHSRPRREMASPPSPLSGEFLDTKDR |
| | | | QAEEDRQMDTEAAASEAPQDVTYAQLHSLTLR |
| | | | REATEPPPSQEGPSPAVPVTYAQLAIH |
| MC0459 | LIR1 | 330 | MALPVTALLLPLALLLHAARPQVQLVQSGAEV |
| (VR459) | (ITM4)X4 | | KKPGASVKVSCKASGYTFTSYHIQWVRQAPGQ |
| | | | RLEWIGWIYPGDGSTQYNEKFKGRVTITRDTSA |
| | | | STAYMELSSLRSEDTAVYYCAREGTYYAMDY |
| | | | WGQGTLVTVSSGGGGSGGGGSGGGGSDVVMT |
| | | | QTPLSLSVTPGQPASISCRSSQSIVHSNGNTYLE |
| | | | WYLQKPGQSPQLLIYKVSNRFSGVPDRFSGSGS |
| | | | GTDFTLKISRVEAEDVGVYYCFQGSHVPRTFGG |
| | | | GTKVEIKTERRAEVPTAHPSPSPRPAGQFQTLVV |
| | | | GVVGGLLGSLVLLVWVLAVILRHRRQGKHWTS |
| | | | TQRKADFQHPAGAVGPEPTDRGLQWRSSPAAD |
| | | | AQEESIYATLKHTQPEDGVEMDTRSPHDEDPQA |
| | | | SIYATLKHSRPRREMASPPSPLSGEFLDTKDRQA |
| | | | EEDRQMDTEAAASEAPQDSIYATLHSLTLRREA |
| | | | TEPPPSQEGPSPAVPSIYATLAIH |
| MC0460 | LIR1 | 33 | MALPVTALLLPLALLLHAARPQVQLVQSGAEV |
| (VR460) | ITIM(3-4) | | KKPGASVKVSCKASGYTFTSYHIQWVRQAPGQ |
| | | | RLEWIGWIYPGDGSTQYNEKFKGRVTITRDTSA |
| | | | STAYMELSSLRSEDTAVYYCAREGTYYAMDY |
| | | | WGQGTLVTVSSGGGGSGGGGSGGGGSDVVMT |
| | | | QTPLSLSVTPGQPASISCRSSQSIVHSNGNTYLE |
| | | | WYLQKPGQSPQLLIYKVSNRFSGVPDRFSGSGS |
| | | | GTDFTLKISRVEAEDVGVYYCFQGSHVPRTFGG |
| | | | GTKVEIKTERRAEVPTAHPSPSPRPAGQFQTLVV |
| | | | GVVGGLLGSLVLLVWVLAVILRHRRQGKHWTS |
| | | | TQRKADFQHPAGAVGPEPTDRGLQWRSSPAAD |
| | | | AQEEVTYAQLKHTQPEDGVEMDTRSPHDEDPQ |
| | | | ASIYATLKHSRPRREMASPPSPLSGEFLDTKDRQ |
| | | | AEEDRQMDTEAAASEAPQDVTYAQLHSLTLRR |
| | | | EATEPPPSQEGPSPAVPSIYATLAIH |
| MC0461 | PD-1ITSM | 332 | MALPVTALLLPLALLLHAARPQVQLVQSGAEV |
| (VR461) | LIR1 | | KKPGASVKVSCKASGYTFTSYHIQWVRQAPGQ |
| | (ITIM3-4) | | RLEWIGWIYPGDGSTQYNEKFKGRVTITRDTSA |
| | | | STAYMELSSLRSEDTAVYYCAREGTYYAMDY |
| | | | WGQGTLVTVSSGGGGSGGGGSGGGGSDVVMT |
| | | | QTPLSLSVTPGQPASISCRSSQSIVHSNGNTYLE |
| | | | WYLQKPGQSPQLLIYKVSNRFSGVPDRFSGSGS |
| | | | GTDFTLKISRVEAEDVGVYYCFQGSHVPRTFGG |
| | | | GTKVEIKTERRAEVPTAHPSPSPRPAGQFQTLVV |
| | | | GVVGGLLGSLVLLVWVLAVILRHRRQGKHWTS |
| | | | TQRKADFQHPAGAVGPEPTDRGLQWRSSPAAD |
| | | | AQEETEYATIKHTQPEDGVEMDTRSPHDEDPQA |
| | | | TEYATIKHSRPRREMASPPSPLSGEFLDTKDRQA |
| | | | EEDRQMDTEAAASEAPQDVTYAQLHSLTLRRE |
| | | | ATEPPPSQEGPSPAVPSIYATLAIH |
| MC0462 | LIR1 | 333 | MALPVTALLLPLALLLHAARPQVQLVQSGAEV |
| (VR462) | (ITIM3-4) | | KKPGASVKVSCKASGYTFTSYHIQWVRQAPGQ |
| | PD- | | RLEWIGWIYPGDGSTQYNEKFKGRVTITRDTSA |
| | 1ITSMX2 | | STAYMELSSLRSEDTAVYYCAREGTYYAMDY |
| | | | WGQGTLVTVSSGGGGSGGGGSGGGGSDVVMT |
| | | | QTPLSLSVTPGQPASISCRSSQSIVHSNGNTYLE |
| | | | WYLQKPGQSPQLLIYKVSNRFSGVPDRFSGSGS |
| | | | GTDFTLKISRVEAEDVGVYYCFQGSHVPRTFGG |
| | | | GTKVEIKTERRAEVPTAHPSPSPRPAGQFQTLVV |
| | | | GVVGGLLGSLVLLVWVLAVILRHRRQGKHWTS |
| | | | TQRKADFQHPAGAVGPEPTDRGLQWRSSPAAD |
| | | | AQEEVTYAQLKHTQPEDGVEMDTRSPHDEDPQ |
| | | | ASIYATLKHSRPRREMASPPSPLSGEFLDTKDRQ |
| | | | AEEDRQMDTEAAASEAPQDTEYATIHSLTLRRE |
| | | | ATEPPPSQEGPSPAVPTEYATIAIH |
| MC0463 | LIR1 | 334 | MALPVTALLLPLALLLHAARPQVQLVQSGAEV |
| (VR463) | ITIM3, | | KKPGASVKVSCKASGYTFTSYHIQWVRQAPGQ |
| | PD-1 | | RLEWIGWIYPGDGSTQYNEKFKGRVTITRDTSA |
| | (ITSM)X2, | | STAYMELSSLRSEDTAVYYCAREGTYYAMDY |
| | LIR1 | | WGQGTLVTVSSGGGGSGGGGSGGGGSDVVMT |
| | ITIM4 | | QTPLSLSVTPGQPASISCRSSQSIVHSNGNTYLE |
| | | | WYLQKPGQSPQLLIYKVSNRFSGVPDRFSGSGS |
| | | | GTDFTLKISRVEAEDVGVYYCFQGSHVPRTFGG |
| | | | GTKVEIKTERRAEVPTAHPSPSPRPAGQFQTLVV |
| | | | GVVGGLLGSLVLLVWVLAVILRHRRQGKHWTS |
| | | | TQRKADFQHPAGAVGPEPTDRGLQWRSSPAAD |
| | | | AQEEVTYAQLKHTQPEDGVEMDTRSPHDEDPQ |
| | | | ATEYATIKHSRPRREMASPPSPLSGEFLDTKDRQ |
| | | | AEEDRQMDTEAAASEAPQDTEYATIHSLTLRRE |
| | | | ATEPPPSQEGPSPAVPSIYATLAIH |

In some embodiments, the WCAR comprises an amino acid sequence selected from the group consisting of SEQ ID NO: 255, SEQ ID NO:256, SEQ ID NO:257, SEQ ID NO:258, SEQ ID NO:305, SEQ ID NO:259, SEQ ID NO:260, SEQ ID NO:261, SEQ ID NO:262, SEQ ID NO:263, SEQ ID NO:264, SEQ ID NO:265, SEQ ID NO:266, SEQ ID NO:267, SEQ ID NO:268, SEQ ID NO:269, SEQ ID NO:270, SEQ ID NO:271, SEQ ID NO:272, SEQ ID NO:327, SEQ ID NO:328, SEQ ID NO:329, SEQ ID NO:330, SEQ ID NO:331, SEQ ID NO:332, SEQ ID NO:333, and SEQ ID NO:334.

7. iCAR Portion/aCAR Portion: Linker

In some embodiments, the iCAR portion is covalently linked to the aCAR portion via a linker. In a certain embodiment, the linker is a gly-ser polypeptide linker, i.e., a peptide that consists of glycine and serine residues. Exemplary gly-ser polypeptide linkers comprise the amino acid sequence Ser(Gly₄Ser)ₙ, as well as (Gly₄Ser)ₙ and/or (Gly₄Ser₃)ₙ. In some embodiments, n=1. In some embodiments, n=2. In some embodiments, n=3, i.e., Ser(Gly₄Ser)₃. In some embodiments, n=4, i.e., Ser(Gly₄Ser)₄. In some embodiments, n=5. In some embodiments, n=6. In some embodiments, n=7. In some embodiments, n=8. In some embodiments, n=9. In some embodiments, n=10. Another exemplary gly-ser polypeptide linker comprises the amino acid sequence Ser(Gly₄Ser)ₙ. In some embodiments, n=1. In some embodiments, n=2. In some embodiments, n=3. In another embodiment, n=4. In some embodiments, n=5. In some embodiments, n=6. Another exemplary gly-ser polypeptide linker comprises (Gly₄Ser)ₙ. In some embodiments, n=1. In some embodiments, n=2. In some embodiments, n=3. In some embodiments, n=4. In some embodiments, n=5. In some embodiments, n=6. Another exemplary gly-ser polypeptide linker comprises (Gly₃Ser)ₙ. In some embodiments, n=1. In some embodiments, n=2. In some embodiments, n=3. In some embodiments, n=4. In another embodiment, n=5. In yet another embodiment, n=6. Another exemplary gly-ser polypeptide linker comprises (Gly₄Ser₃)ₙ. In some embodiments, n=1. In some embodiments, n=2. In some embodiments, n=3. In some embodiments, n=4. In some embodiments, n=5. In some embodiments, n=6. Another exemplary gly-ser polypeptide linker comprises (Gly₃Ser)ₙ. In some embodiments, n=1. In some embodiments, n=2. In some embodiments, n=3. In some embodiments, n=4. In another embodiment, n=5. In yet another embodiment, n=6.

In some embodiments, the bicistronic construct comprises a linker that covalently connects the iCAR portion and the aCAR portion. In some embodiments, the bicistronic construct comprises a viral self-cleaving 2A peptide between the nucleic acid sequence encoding the iCAR portion and the nucleic acid sequence encoding the aCAR portion of the construct. In some embodiments, the viral self-cleaving 2A peptide includes T2A from Thosea asigna virus (TaV). In some embodiments, the iCAR portion is covalently linked to the aCAR portion via a linker. In some embodiments, the iCAR portion is covalently linked to the aCAR portion via a GSG. In some embodiments, the iCAR portion is covalently linked to the aCAR portion via a GGGGS linker (SEQ ID NO:153). In some embodiments, the iCAR portion is covalently linked to the aCAR portion via a GGGGSGGGGSGGGGS linker (SEQ ID NO:154). In some embodiments, the iCAR is covalently linked to the aCAR portion via a T2A linker (SEQ ID NO:155). In some embodiments, the iCAR is covalently linked to the aCAR portion via a F2A linker (SEQ ID NO:156). In some embodiments, the iCAR is covalently linked to the aCAR portion via a P2A linker (SEQ ID NO:157). In some embodiments, the iCAR is covalently linked to the aCAR portion via a E2A linker (SEQ ID NO:158). In some embodiments, the iCAR is covalently linked to the aCAR portion via a IRES long linker (SEQ ID NO:159). In some embodiments, the iCAR is covalently linked to the aCAR portion via a IRES short linker (SEQ ID NO:160).

TABLE 13
iCAR portion/aCAR portion linker sequences

| Sequence | | |
|---|---|---|
| Information | SEQ ID NO | Amino acid sequence |
| G₄S | 153 | GGGGS |
| (G₄S)X3 | 154 | GGGGSGGGGSGGGGS |
| T2A | 155 | GSGEGRGSLLTCGDVEENPGP |
| F2A | 156 | GSGVKQTLNFDLLKLAGDVESNPGP |
| P2A | 157 | GSGATNFSLLKQAGDVEENPGP |
| E2A | 158 | GSGQCTNYALLKLAGDVESNPGP |
| IRES long | 159 | CCCCCCCCCCTAACGTTACTGGCCGAAGCCGCTT |
| | | GGAATAAGGCCGGTGTGCGTTTGTCTATATGTTA |
| | | TTTTCCACCATATTGCCGTCTTTTGGCAATGTGAG |
| | | GGCCCGGAAACCTGGCCCTGTCTTCTTGACGAGC |
| | | ATTCCTAGGGGTCTTTCCCCTCTCGCCAAAGGAA |
| | | TGCAAGGTCTGTTGAATGTCGTGAAGGAAGCAGT |
| | | TCCTCTGGAAGCTTCTTGAAGACAAACAACGTCT |
| | | GTAGCGACCCTTTGCAGGCAGCGGAACCCCCCAC |
| | | CTGGCGACAGGTGCCTCTGCGGCCAAAAGCCACG |
| | | TGTATAAGATACACCTGCAAAGGCGGCACAACCC |
| | | CAGTGCCACGTTGTGAGTTGGATAGTTGTGGAAA |
| | | GAGTCAAATGGCTCTCCTCAAGCGTATTCAACAA |
| | | GGGGCTGAAGGATGCCCAGAAGGTACCCCATTGT |
| | | ATGGGATCTGATCTGGGGCCTCGGTGCACATGCT |
| | | TTACATGTGTTTAGTCGAGGTTAAAAAAACGTCT |
| | | AGGCCCCCCGAACCACGGGGACGTGGTTTTCCTT |
| | | TGAAAAACACGATGATAATATG |
| IRES short | 160 | CCCCTCTCGCCAAAGGAATGCAAGGTCTGTTGAA |
| | | TGTCGTGAAGGAAGCAGTTCCTCTGGAAGCTTCT |
| | | TGAAGACAAACAACGTCTGTAGCGACCCTTTGCA |
| | | GGCAGCGGAACCCCCCACCTGGCGACAGGTGCCT |
| | | CTGCGGCCAAAAGCCACGTGTATAAGATACACCT |
| | | GCAAAGGCGGCACAACCCCAGTGCCACGTTGTG |
| | | AGTTGGATAGTTGTGGAAAGAGTCAAATGGCTCT |
| | | CCTCAAGCGTATTCAACAAGGGGCTGAAGGATGC |
| | | CCAGAAGGTACCCCATTGTATGGGATCTGATCTG |
| | | GGGCCTCGGTGCACATGCTTTACATGTGTTTAGT |
| | | CGAGGTTAAAAAAACGTCTAGGCCCCCCGAACC |
| | | ACGGGGACGTGGTTTTCCTTTGAAAAACACGATG |
| | | ATAATATG |

8. iCAR Portion/aCAR Portion: Signal Peptide

In some embodiments, the bicistronic construct comprises a signal peptide upstream of the iCAR and aCAR portions. In some embodiments, the signal peptide is a CD8 alpha signal peptide (SEQ ID NO: 161). In some embodiments, the signal peptide is a GM-CSF signal peptide (SEQ ID NO: 162). In some embodiments, the signal peptide is a mIgK signal peptide (SEQ ID NO: 306).

TABLE 14
iCAR/aCAR signal peptide sequences

| Sequence | | |
|---|---|---|
| Information | SEQ ID NO | Amino acid sequence |
| CD8 alpha | 161 | MALPVTALLLPLALLLHAARP |
| GM-CSF | 162 | MLLLVTSLLLCELPHPAFLLIP |
| mIgK | 306 | MSVPTQVLGLLLLWLTDARC |

9. aCAR Portion: aCAR Scfv

In some embodiments, the bicistronic construct comprises an aCAR portion comprising a single chain variable fragment (scFv) component. In some embodiments, the iCAR portion comprises an scFv component. In some embodiments, the scFv targets Her2, Mesothelin, or EGFR. In some embodiments, the scFv targets Her2. In some embodiments, the scFv targets Mesothelin. In some embodiments, the scFv targets EGFR. In some embodiments, the scFv is an scFv based on trastuzumab (anti-Her2 antibody, also referred to as HERCEPTIN®), pertuzumab (anti-Her2 antibody, also referred to as PERJETA®), another commercial anti-Her2 antibody including, but not limited to, FRP5, A21, XMT1517, XMT1518, XMT1519, FWP51, bioequivalents thereof, or biosimilars thereof. In some embodiments, the scFv has the VH and VL domains of trastuzumab, pertuzumab, FRP5, A21, XMT1517, XMT1518, XMT1519, FWP51, bioequivalents thereof, or biosimilars thereof. In some embodiments, the scFv is an scFv based on cetuximab (anti-EGFR antibody, also referred to as ERBITUX®), panitumumab (anti-EGFR antibody, also referred to as VECTIBIX®), another commercial anti-EGFR antibody including, but not limited to, Imgatuzumab, Nimotuzumab, Necitumumab, ICR62, Matuzumab, C10, Zalutumumab, P1X, P2X, P3X, EGFR-1a1-VHH, bioequivalents thereof, or biosimilars thereof. In some embodiments, the scFv has the VH and VL domains of cetuximab, panitumumab, Imgatuzumab, Nimotuzumab, Necitumumab, ICR62, Matuzumab, C10, Zalutumumab, P1X, P2X, P3X, EGFR-1a1-VHH, bioequivalents thereof, or biosimilars thereof. In some embodiments, the scFv is an scFv based on a commercial anti-Mesothelin antibody including, but not limited to, Amatuximab, P4, SS1, SD1, SD2, 11H7, 3C02, bioequivalents thereof, or biosimilars thereof. In some embodiments, the scFv has the VH and VL domains of Amatuximab, P4, SS1, SD1, SD2, 1 e7, 3C02, bioequivalents thereof, or biosimilars thereof.

In some embodiments, the scFv targets Her2. In some embodiments, the Her2 scFv is based on the Vh and Vl from trastuzumab or pertuzumab. In some embodiments, the Her2 scFv is based on the Vh and Vl from trastuzumab. In some embodiments, the Her2 scFv is based on the Vh and Vl from pertuzumab. The Vh and Vl chains for trastuzumab and pertuzumab are provided below in Tables 15 and 16. In some embodiments, the Her2 scFv is based on the Vh and Vl from FRP5. In some embodiments, the Her2 scFv is based on the Vh and Vl from A21. In some embodiments, the Her2 scFv is based on the Vh and Vl from XMT1517. In some embodiments, the Her2 scFv is based on the Vh and Vl from XMT1518. In some embodiments, the Her2 scFv is based on the Vh and Vl from XMT1519. In some embodiments, the Her2 scFv is based on the Vh and Vl from FWP51. In some embodiments, the Her2 scFv is based on the Vh and Vl from trastuzumab F9G.

TABLE 15
anti-Her2 sequences

| | SEQ | |
|---|---|---|
| Sequence | ID | |
| Information | NO | Amino acid sequence |
| trastuzumab | 170 | EVQLVESGGGLVQPGGSLRLSCAASGFNIKDTYIHWVRQ |
| Variable heavy | | APGKGLEWVARIYPTNGYTRYADSVKGRFTISADTSKNT |
| chain | | AYLQMNSLRAEDTAVYYCSRWGGDGFYAMDYWGQGT |
| | | LVTVSS |
| trastuzumab | 171 | DIQMTQSPSSLSASVGDRVTITCRASQDVNTAVAWYQQK |
| Variable light | | PGKAPKLLIYSASFLYSGVPSRFSGSRSGTDFTLTISSLQPE |
| chain | | DFATYYCQQHYTTPPTFGQGTKVEIK |
| trastuzumab | 172 | EVQLVESGGGLVQPGGSLRLSCAASGFNIKDTYIHWVRQ |
| scFv | | APGKGLEWVARIYPTNGYTRYADSVKGRFTISADTSKNT |
| | | AYLQMNSLRAEDTAVYYCSRWGGDGFYAMDYWGQGT |
| | | LVTVSSGSTSGSGKPGSGEGSTKGDIQMTQSPSSLSASV |
| | | GDRVTITCRASQDVNTAVAWYQQKPGKAPKLLIYSASFL |
| | | YSGVPSRFSGSRSGTDFTLTISSLQPEDFATYYCQQHYTTP |
| | | PTFGQGTKVEIK |
| Trastuzumab | 307 | DIQMTQSPSSLSASVGDRVTITCRASQDVNTAVAWYQQK |
| F9G variable | | PGKAPKLLIYSASFLYSGVPSRFSGSRSGTDFTLTISSLQPE |
| Heavy chain | | DFATYYCQQHYTTPPTFGQGTKVEIK |
| Trastuzumab | 308 | EVQLVESGGGLVQPGGSLRLSCAASGFNIKDTYIHWVRQ |
| F9G variable | | APGKGLEWVARIYPTNGYTRYADSVKGRFTISADTSKNT |
| Light chain | | AYLQMNSLRAEDTAVYYCSRWGGDGGYAMDYWGQGT |
| | | LVTVSS |
| pertuzumab | 173 | EVQLVESGGGLVQPGGSLRLSCAASGFTFTDYTMDWVR |
| Variable heavy | | QAPGKGLEWVADVNPNSGGSIYNQRFKGRFTLSVDRSK |
| chain | | NTLYLQMNSLRAEDTAVYYCARNLGPSFYFDYWGQGTL |
| | | VTVSS |
| pertuzumab | 174 | DIQMTQSPSSLSASVGDRVTITCKASQDVSIGVAWYQQK |
| Variable light | | PGKAPKLLIYSASYRYTGVPSRFSGSGSGTDFTLTISSLQP |
| chain | | EDFATYYCQQYYIYPYTFGQGTKVEIK |
| pertuzumab scFv | 175 | DIQMTQSPSSLSASVGDRVTITCKASQDVSIGVAWYQQK |
| | | PGKAPKLLIYSASYRYTGVPSRFSGSGSGTDFTLTISSLQP |
| | | EDFATYYCQQYYIYPYTFGQGTKVEIKGSTSGSGKPGSGE |
| | | GSTKGEVQLVESGGGLVQPGGSLRLSCAASGFTFTDYTM |
| | | DWVRQAPGKGLEWVADVNPNSGGSIYNQRFKGRFTLSV |
| | | DRSKNTLYLQMNSLRAEDTAVYYCARNLGPSFYFDYWG |
| | | QGTLVTVSS |
| FRP5 variable | 176 | QVQLQQSGPELKKPGETVKISCKASGYPFTNYGMNWVK |
| heavy chain | | QAPGQGLKWMGWINTSTGESTFADDFKGRFDFSLETSAN |
| | | TAYLQINNLKSEDMATYFCARWEVYHGYVPYWGQGTT |
| | | VTVSS |
| FRP5 variable | 177 | DIQLTQSHKFLSTSVGDRVSITCKASQDVYNAVAWYQQK |
| light chain | | PGQSPKLLIYSASSRYTGVPSRFTGSGSGPDFTFTISSVQA |
| | | EDLAVYFCQQHFRTPFTFGSGTKLEIK |
| A21 variable | 178 | EVQLQQSGPEVVKTGASVKISCKASGYSFTGYFINWVKK |
| heavy chain | | NSGKSPEWIGHISSSYATSTYNQKFKNKAAFTVDTSSSTA |
| | | FMQLNSLTSEDSAVYYCVRSGNYEEYAMDYWGQGTSVT |
| | | VSS |
| A21 variable | 179 | DIVLTQTPSSLPVSVGEKVTMTCKSSQTLLYSNNQKNYL |
| light chain | | AWYQQKPGQSPKLLISWAFTRKSGVPDRFTGSGSGTDFT |
| | | LTIGSVKAEDLAVYYCQQYSNYPWTFGGGTKLEIK |
| XMT1517 | 180 | QVQLVESGGGVVQPGRSLRLSCAASGFTFSSYGMHWVR |
| variable heavy | | QAPGKGLEWVAVIWYDGSNKYYADSVKGRFTISRDNSK |
| chain | | NTLYLQMNSLRAEDTAVYYCAKEAPYYAKDYMDVWG |
| | | KGTTVTVSS |
| XMT1517 | 181 | EIVLTQSPGTLSLSPGERATLSCRASQSVSSDYLAWYQQK |
| variable light | | PGQAPRLLIYGASSRATGIPDRFSGSGSGTDFTLTISRLEPE |
| chain | | DFAVYYCQQYVSYWTFGGGTKVEIK |
| XMT1518 | 182 | QVQLVESGGGVVQPGRSLRLSCAASGFTFSSYGMHWVR |
| variable heavy | | QAPGKGLEWVAGIWWDGSNEKYADSVKGRFTISRDNSK |
| chain | | NTLYLQMNSLRAEDTAVYYCAKEAPYYAKDYMDVWG |
| | | KGTTVTVSS |
| XMT1518 | 183 | EIVLTQSPGTLSLSPGERATLSCRASQSVSSDYLAWYQQK |
| variable light | | PGQAPRLLIYGASRRATGIPDRFSGSGSGTDFTLTISRLEPE |
| chain | | DFAVYYCQQYVSYWTFGGGTKVEIK |
| XMT1519 | 184 | EVQLVESGGGLVQPGGSLRLSCAASGFTFSSYSMNWVRQ |
| variable heavy | | APGKGLEWVSYISSSSSTIYYADSVKGRFTISRDNAKNSL |
| chain | | YLQMNSLRAEDTAVYYCARGGHGYFDLWGRGTLVTVS |
| | | S |
| XMT1519 | 185 | EIVLTQSPGTLSLSPGERATLSCRASQSVSSSYLAWYQQK |
| variable light | | PGQAPRLLIYGASSRATGIPDRFSGSGSGTDFTLTISRLEPE |
| chain | | DFAVYYCQQYHHSPLTFGGGTKVEIK |
| FWP51 variable | 186 | QVQLQQSGAELVRPGTSVKLSCKASDYTFTSYWMNWVK |
| heavy chain | | QRPGQGLEWIGMIDPSDSETQYNQMFKDKAALTVDKSS |
| | | NTAYMQLSSLTSEDSAVYYCAKGGASGDWYFDVWGQG |
| | | TTVT |
| FWP51 variable | 187 | DIQLTQSPSSLSASLGGEVTITCKASQDIKKYIAWYQHKP |
| light chain | | GKSPRLLIHYTSVLQPGIPSRFSGSGSGRDYSFSIHNLEPED |
| | | IATYYCLHYDYLYTFGGGTKLEI |
| FWP51 VL VH | 188 | MLLLVTSLLLCELPHPAFLLIPDYKDDDDKQVQLQQSGA |
| | | ELVRPGTSVKLSCKASDYTFTSYWMNWVKQRPGQGLE |
| | | WIGMIDPSDSETQYNQMFKDKAALTVDKSSNTAYMQLS |
| | | SLTSEDSAVYYCAKGGASGDWYFDVWGQGTTVTGSTSG |
| | | SGKPGSGEGSTKGDIQLTQSPSSLSASLGGEVTITCKASQD |
| | | IKKYIAWYQHKPGKSPRLLIHYTSVLQPGIPSRFSGSGSGR |
| | | DYSFSIHNLEPEDIATYYCLHYDYLYTFGGGTKLEI |
| Anti HER2 VHH | 309 | QVQLVQSGGGLVQAGGSLRLSCAASGRTFSSYAMAWFR |
| | | QAPGKEREFVAAISWSGANIYVADSVKGRFTISRDNAKD |
| | | TVYLQMNSLKPEDTAVYYCAVKLGFAPVEERQYDYWG |
| | | QGTQVTVSS |

In some embodiments, the scFv targets EGFR. In some embodiments, the EGFR scFv is based on the Vh and Vl from cetuximab, panitumumab, Jmgatuzumab, Nimotuzumab, Necitumumab, ICR62, Matuzumab, C10, Zalutumumab, P1X, P2X, P3X, or EGFR-1a1-VHH. In some embodiments, the EGFR scFv is based on the Vh and Vl from cetuximab. In some embodiments, the EGFR scFv is based on the Vh and Vl from panitumumab. In some embodiments, the EGFR scFv is based on the Vh and Vl from Jmgatuzumab. In some embodiments, the EGFR scFv is based on the Vh and Vl from Nimotuzumab. In some embodiments, the EGFR scFv is based on the Vh and Vl from Nimotuzumab (K5). In some embodiments, the EGFR scFv is based on the Vh and Vl from Necitumumab. In some embodiments, the EGFR scFv is based on the Vh and Vl from ICR62. In some embodiments, the EGFR scFv is based on the Vh and Vl from Matuzumab. In some embodiments, the EGFR scFv is based on the Vh and Vl from C10. In some embodiments, the EGFR scFv is based on the Vh and Vl from Zalutumumab. In some embodiments, the EGFR scFv is based on the Vh and Vl from P1X. In some embodiments, the EGFR scFv is based on the Vh and Vl from P2X. In some embodiments, the EGFR scFv is based on the Vh and Vl from P3X. In some embodiments, the EGFR scFv is based on EGFR-1a1-VHH. In some embodiments, the EGFR scFv is based on EGFR-VHH.

TABLE 16
anti-EGFR sequences

| Sequence | SEQ ID | |
|---|---|---|
| Information | NO | Amino acid sequence |
| cetuximab | 189 | QVQLKQSGPGLVQPSQSLSITCTVSGFSLTNYGVHWVRQ |
| Variable heavy | | SPGKGLEWLGVIWSGGNTDYNTPFTSRLSINKDNSKSQV |
| chain | | FFKMNSLQSNDTAIYYCARALTYYDYEFAYWGQGTLVT |
| | | VS |
| cetuximab | 190 | DILLTQSPVILSVSPGERVSFSCRASQSIGTNIHWYQQRTN |
| Variable light | | GSPRLLIKYASESISGIPSRFSGSGSGTDFTLSINSVESEDIA |
| chain | | DYYCQQNNNWPTTFGAGTKLELK |
| cetuximab scFv | 191 | QVQLKQSGPGLVQPSQSLSITCTVSGFSLTNYGVHWVRQ |
| (SEQ ID NO:) | | SPGKGLEWLGVIWSGGNTDYNTPFTSRLSINKDNSKSQV |
| | | FFKMNSLQSNDTAIYYCARALTYYDYEFAYWGQGTLVT |
| | | VSGSTSGSGKPGSGEGSTKGDILLTQSPVILSVSPGERVSF |
| | | SCRASQSIGTNIHWYQQRTNGSPRLLIKYASESISGIPSRF |
| | | SGSGSGTDFTLSINSVESEDIADYYCQQNNNWPTTFGAG |
| | | TKLELK |
| panitumumab | 192 | QVQLQESGPGLVKPSETLSLTCTVSGGSVSSGDYYWTWI |
| Variable heavy | | RQSPGKGLEWIGHIYYSGNTNYNPSLKSRLTISIDTSKTQ |
| chain | | FSLKLSSVTAADTAIYYCVRDRVTGAFDIWGQGTMVTV |
| | | SS |
| panitumumab | 193 | DIQMTQSPSSLSASVGDRVTITCQASQDISNYLNWYQQK |
| Variable light | | PGKAPKLLIYDASNLETGVPSRFSGSGSGTDFTFTISSLQP |
| chain | | EDIATYFCQHFDHLPLAFGGGTKVEIK |
| panitumumab | 194 | DIQMTQSPSSLSASVGDRVTITCQASQDISNYLNWYQQK |
| scFv | | PGKAPKLLIYDASNLETGVPSRFSGSGSGTDFTFTISSLQP |
| | | EDIATYFCQHFDHLPLAFGGGTKVEIKGSTSGSGKPGSGE |
| | | GSTKGQVQLQESGPGLVKPSETLSLTCTVSGGSVSSGDY |
| | | YWTWIRQSPGKGLEWIGHIYYSGNTNYNPSLKSRLTISID |
| | | TSKTQFSLKLSSVTAADTAIYYCVRDRVTGAFDIWGQGT |
| | | MVTVSS |
| Imgatuzuma | 195 | QVQLVQSGAEVKKPGSSVKVSCKASGFTFTDYKIHWVR |
| variable heavy | | QAPGQGLEWMGYFNPNSGYSTYAQKFQGRVTITADKST |
| chain | | STAYMELSSLRSEDTAVYYCARLSPGGYYVMDAWGQG |
| | | TTVTVSS |
| Imgatuzumab | 196 | DIQMTQSPSSLSASVGDRVTITCRASQGINNYLNWYQQK |
| variable light | | PGKAPKRLIYNTNNLQTGVPSRFSGSGSGTEFTLTISSLQP |
| chain | | EDFATYYCLQHNSFPTFGQGTKLEIK |
| Nimotuzumab | 197 | QVQLQQSGAEVKKPGSSVKVSCKASGYTFTNYYIYWVR |
| variable heavy | | QAPGQGLEWIGGINPTSGGSNFNEKFKTRVTITVDESTNT |
| chain | | AYMELSSLRSEDTAFYFCARQGLWFDSDGRGFDFWGQG |
| | | STVTVSS |
| Nimotuzumab | 198 | DIQMTQSPSSLSASVGDRVTITCRSSQNIVHSNGNTYLD |
| variable light | | WYQQTPGKAPKLLIYKVSNRFSGVPSRFSGSGSGTDFTF |
| chain | | TISSLQPEDIATYYCFQYSHVPWTFGQGTKLQIT |
| Nimotuzumab | 310 | DIQMTQSPSSLSASVGDRVTITCRSSQNIVHSNGNTYLD |
| (K5) variable | | WYQQTPGKAPKLLIYKVSNRFSGVPSRFSGSGSGTDFTF |
| light chain | | TISSLQPEDIATYYCFQYSHVPWTFGQGTKLQIT |
| Nimotuzumab | 311 | QVQLQQSGAEVKKPGSSVKVSCKASGYTFTDYYIYWVR |
| (K5) variable | | QAPGQGLEWIGGINPVTQRPVFNEKFKTRVTITVDESTNT |
| Heavy chain | | AYMELSSLRSEDTAFYFCARQGLWFDSDGRGFDFWGQG |
| | | STVTVSS |
| Necitumumab | 199 | QVQLQESGPGLVKPSQTLSLTCTVSGGSISSGDYYWSWI |
| variable heavy | | RQPPGKGLEWIGYIYYSGSTDYNPSLKSRVTMSVDTSKN |
| chain | | QFSLKVNSVTAADTAVYYCARVSIFGVGTFDYWGQGTL |
| | | VTVSS |
| Necitumumab | 200 | EIVMTQSPATLSLSPGERATLSCRASQSVSSYLAWYQQK |
| variable light | | PGQAPRLLIYDASNRATGIPARFSGSGSGTDFTLTISSLEP |
| chain | | EDFAVYYCHQYGSTPLTFGGGTKAEIK |
| ICR62 variable | 201 | QVNLLQSGAALVKPGASVKLSCKGSGFTFTDYKIHWVK |
| heavy chain | | QSHGKSLEWIGYFNPNSGYSTYNEKFKSKATLTADKSTD |
| | | TAYMELTSLTSEDSATYYCTRLSPGGYYVMDAWGQGA |
| | | SVTVSS |
| ICR62 variable | 202 | DIQMTQSPSFLSASVGDRVTINCKASQNINNYLNWYQQK |
| light chain | | LGEAPKRLIYNTNNLQTGIPSRFSGSGSGTDYTLTISSLQP |
| | | EDFATYFCLQHNSFPTFGAGTKLELK |
| ICR62 VL VH | 203 | MLLLVTSLLLCELPHPAFLLIPDIQMTQSPSFLSASVGDR |
| | | VTINCKASQNINNYLNWYQQKLGEAPKRLIYNTNNLQT |
| | | GIPSRFSGSGSGTDYTLTISSLQPEDFATYFCLQHNSFPTF |
| | | GAGTKLELKGSTSGSGKPGSGEGSTKGQVNLLQSGAAL |
| | | VKPGASVKLSCKGSGFTFTDYKIHWVKQSHGKSLEWIG |
| | | YFNPNSGYSTYNEKFKSKATLTADKSTDTAYMELTSLTS |
| | | EDSATYYCTRLSPGGYYVMDAWGQGASVTVSS |
| Matuzumab | 204 | QVQLVQSGAEVKKPGASVKVSCKASGYTFTSHWMHWV |
| variable heavy | | RQAPGQGLEWIGEFNPSNGRTNYNEKFKSKATMTVDTS |
| chain | | TNTAYMELSSLRSEDTAVYYCASRDYDYDGRYFDYWG |
| | | QGTLVTVSS |
| Matuzumab | 205 | DIQMTQSPSSLSASVGDRVTITCSASSSVTYMYWYQQKP |
| variable light | | GKAPKLLIYDTSNLASGVPSRFSGSGSGTDYTFTISSLQPE |
| chain | | DIATYYCQQWSSHIFTFGQGTKVEIK |
| C10 variable | 206 | EVQLVQSGAEVKKPGSSVKVSCKASGGTFSSYAIGWVR |
| heavy chain | | QAPGQGLEWMGGIIPIFGIANYAQKFQGRVTITADESTSS |
| | | AYMELSSLRSEDTAVYYCAREEGPYCSSTSCYAAFDIWG |
| | | QGTLVTLSS |
| C10 variable | 207 | QSVLTQDPAVSVALGQTVKITCQGDSLRSYFASWYQQK |
| light chain | | PGQAPTLVMYARNDRPAGVPDRFSGSKSGTSASLSAISG |
| | | LQPEDEAYYCAAWDDSLNGYLFGAGTKLTVL |
| Zalutumumab | 208 | QVQLVESGGGVVQPGRSLRLSCAASGFTFSTYGMHWVR |
| variable heavy | | QAPGKGLEWVAVIWDDGSYKYYGDSVKGRFTISRDNSK |
| chain | | NTLYLQMNSLRAEDTAVYYCARDGITMVRGVMKDYFD |
| | | YWGQGTLVTVSS |
| Zalutumumab | 209 | AIQLTQSPSSLSASVGDRVTITCRASQDISSALVWYQQKP |
| variable light | | GKAPKLLIYDASSLESGVPSRFSGSESGTDFTLTISSLQPE |
| chain | | DFATYYCQQFNSYPLTFGGGTKVEIK |
| P1X variable | 210 | QVQLVQSGAEVKKPGSSVKVSCKASGGTFSSYAISWVR |
| heavy chain | | QAPGQGLEWMGSIIPIFGTVNYAQKFQGRVTITADESTST |
| | | AYMELSSLRSEDTAVYYCARDPSVNLYWYFDLWGRGT |
| | | LVTVSS |
| P1X variable | 211 | DIQMTQSPSTLSASVGDRVTITCRASQSISSWWAWYQQK |
| light chain | | PGKAPKLLIYDASSLESGVPSRFSGSGSGTEFTLTISSLQP |
| | | DDFATYYCQQYHAHPTTFGGGTKVEIK |
| P2X variable | 212 | QVQLVQSGAEVKKPGSSVKVSCKASGGTFGSYAISWVR |
| heavy chain | | QAPGQGLEWMGSIIPIFGAANPAQKSQGRVTITADESTST |
| | | AYMELSSLRSEDTAVYYCAKMGRGKVAFDIWGQGTMV |
| | | TVSS |
| P2X variable | 213 | DIVMTQSPDSLAVSLGERATINCKSSQSVLYSPNNKNYL |
| light chain | | AWYQQKPGQPPKLLIYWASTRESGVPDRFSGSGSGTDFT |
| | | LTISSLQAEDVAVYYCQQYYGSPITFGGGTKVEIK |
| P3X variable | 214 | QVQLVQSGAEVKKPGASVKVSCKASGYAFTSYGINWVR |
| heavy chain | | QAPGQGLEWMGWISAYNGNTYYAQKLRGRVTMTTDTS |
| | | TSTAYMELRSLRSDDTAVYYCARDLGGYGSGSVPFDPW |
| | | GQGTLVTVSS |
| P3X variable | 215 | EIVMTQSPATLSVSPGERATLSCRASQSVSSNLAWYQQK |
| light chain | | PGQAPRLLIYGASTRATGIPARFSGSGSGTEFTLTISSLQS |
| | | EDFAVYYCQDYRTWPRRVFGGGTKVEIK |
| EGFR-1a1-VHH | 216 | QVQLQESGGGLVQAGGSLLLSCAASGRTFSSYAMGWFR |
| variable heavy | | QAPGKEREFVAAINWSGGSTSYADSVKGRFTISRDNTKN |
| chain | | TVYLQMNSLKPEDTAAFYCAATYNPYSRDHYFPRMTTE |
| | | YDYWGQGTQVTVSS |
| EGFR-VHH | 312 | EVQQASGGGLVQAGGSLRLSCAASGRTETTSAIAWFRQ |
| variable heavy | | APGKEREFVAQISASGLGINYSGTVKGRFTISRDADKTTV |
| chain | | YLQMNSLTPEDTAVYYCAAGFHYIAAIRRTTDFHFWGP |
| | | GTLVTVSS |

In some embodiments, the scFv targets Mesothelin. In some embodiments, the Mesothelin scFv is based on the Vh and Vl from Amatuximab, P4, SS1, SD1, SD2, 1H7, or 3C02. In some embodiments, the Mesothelin scFv is based on the Vh and Vl from Amatuximab. In some embodiments, the Mesothelin scFv is based on the Vh and Vl from P4. In some embodiments, the Mesothelin scFv is based on the Vh and Vl from SS1. In some embodiments, the Mesothelin scFv is based on SD1. In some embodiments, the Mesothelin scFv is based on SD2. In some embodiments, the Mesothelin scFv is based on the Vh and Vl from 1H7. In some embodiments, the Mesothelin scFv is based on the Vh and Vl from 3C02.

TABLE 17
anti-Mesothelin sequences

| Sequence | SEQ ID | |
|---|---|---|
| Information | NO | Amino acid sequence |
| Amatuximab | 217 | QVQLVQSGAEVKKPGASVKVSCKASGYSFTGYTMNWV |
| variable heavy | | RQAPGQGLEWMGLITPYNGASSYNQKFRGKATMTVDTS |
| chain | | TSTVYMELSSLRSEDTAVYYCARGGYDGRGFDYWGQG |
| | | TLVTVSS |
| Amatuximab | 218 | DIQMTQSPSSLSASVGDRVTITCSASSSVSYMHWYQQKS |
| variable light | | GKAPKLLIYDTSKLASGVPSRFSGSGSGTDFTLTISSLQPE |
| chain | | DFATYYCQQWSKHPLTFGQGTKLEIK |
| P4 variable | 219 | QVQLQQSGPGLVTPSQTLSLTCAISGDSVSSNSATWNWI |
| heavy chain | | RQSPSRGLEWLGRTYYRSKWYNDYAVSVKSRMSINPDT |
| | | SKNQFSLQLNSVTPEDTAVYYCARGMMTYYYGMDVW |
| | | GQGTTVTVSS |
| P4 variable light | 220 | QPVLTQSSSLSASPGASASLTCTLRSGINVGPYRIYWYQQ |
| chain | | KPGSPPQYLLNYKSDSDKQQGSGVPSRFSGSKDASANA |
| | | GVLLISGLRSEDEADYYCMIWHSSAAVFGGGTQLTVL |
| P4 VL VH | 221 | MLLLVTSLLLCELPHPAFLLIPQPVLTQSSSLSASPGASAS |
| | | LTCTLRSGINVGPYRIYWYQQKPGSPPQYLLNYKSDSDK |
| | | QQGSGVPSRFSGSKDASANAGVLLISGLRSEDEADYYC |
| | | MIWHSSAAVFGGGTQLTVLGSTSGSGKPGSGEGSTKGQ |
| | | VQLQQSGPGLVTPSQTLSLTCAISGDSVSSNSATWNWIR |
| | | QSPSRGLEWLGRTYYRSKWYNDYAVSVKSRMSINPDTS |
| | | KNQFSLQLNSVTPEDTAVYYCARGMMTYYYGMDVWG |
| | | QGTTVTVSS |
| SS1 variable | 222 | QVQLQQSGPELEKPGASVKISCKASGYSFTGYTMNWVK |
| heavy chain | | QSHGKSLEWIGLITPYNGASSYNQKFRGKATLTVDKSSS |
| | | TAYMDLLSLTSEDSAVYFCARGGYDGRGFDYWGSGTPV |
| | | TVSS |
| SS1 variable | 223 | DIELTQSPAIMSASPGEKVTMTCSASSSVSYMHWYQQKS |
| light chain | | GTSPKRWIYDTSKLASGVPGRFSGSGSGNSYSLTISSVEA |
| | | EDDATYYCQQWSKHPLTFGSGTKVEIK |
| SS1 VL VH | 224 | MLLLVTSLLLCELPHPDIELTQSPAIMSASPGEKVTMTCS |
| | | ASSSVSYMHWYQQKSGTSPKRWIYDTSKLASGVPGRES |
| | | GSGSGNSYSLTISSVEAEDDATYYCQQWSKHPLTFGSGT |
| | | KVEIKGSTSGSGKPGSGEGSTKGQVQLQQSGPELEKPGA |
| | | SVKISCKASGYSFTGYTMNWVKQSHGKSLEWIGLITPYN |
| | | GASSYNQKFRGKATLTVDKSSSTAYMDLLSLTSEDSAV |
| | | YFCARGGYDGRGFDYWGSGTPVTVSS |
| SD1 VHH | 225 | QVQLVQSGGGLVQPGGSLRLSCAASDFDFAAYEMSWV |
| | | RQSAPGQGLEWVAIISHDGIDKYYTDSVKGRFTISRDNS |
| | | KNTLYLQMNTLRAEDTATYYCLRLGAVGQGTLVTVSSS |
| SD2 VHH | 226 | QVQLVQSGGGLVQPGGSLRLSCAASDFAFDDYEMSWV |
| | | RQAPGKALEWIGDINHSGTTIYNPSLKSRVTISRDNSKNT |
| | | LYLQMNTLRAEDTAIYYCARPHYGDYSDAFDIWGQGT |
| | | MVTVSS |
| 1H7 variable | 227 | EVQLQQSGTVLARPGASVKMSCKASGYSFTNYRMNWV |
| heavy chain | | KQRPGQGLEWIGGIYPGNRDTTYNQKFKDKAKLTAVTS |
| | | ANTAYMELSSLTNEDSAVYYCTRGVIGIYFDYWGQGTT |
| | | LTVSS |
| 1H7 variable | 228 | DIVMTQSPASLAVSLGQRATISCKASQSVDYDGDSYMN |
| light chain | | WYQQKPGQPPKLLIYAASNLESGIPARFSGSGSGTDFTLN |
| | | IHPVEEEDAATYYCQQNNEAPLTFGAGTKLELK |
| 1H7 VL VH | 229 | MLLLVTSLLLCELPHPAFLLIPDIVMTQSPASLAVSLGQR |
| | | ATISCKASQSVDYDGDSYMNWYQQKPGQPPKLLIYAAS |
| | | NLESGIPARFSGSGSGTDFTLNIHPVEEEDAATYYCQQNN |
| | | EAPLTFGAGTKLELKGSTSGSGKPGSGEGSTKGEVQLQQ |
| | | SGTVLARPGASVKMSCKASGYSFTNYRMNWVKQRPGQ |
| | | GLEWIGGIYPGNRDTTYNQKFKDKAKLTAVTSANTAYM |
| | | ELSSLTNEDSAVYYCTRGVIGIYFDYWGQGTTLTVSS |
| 3C02 variable | 230 | QVQLQQSGTVLARPGASVKMSCKASGYSFTNYRMYWV |
| heavy chain | | KQRPGQGLEWIGAIYPGNSDTTYKQKFKGKAKLTAVTS |
| | | ASTAYMELSSLTNEDSAVYYCTRGIRGSYFDVWGAGTT |
| | | VTVSS |
| 3C02 variable | 231 | DIVMTQSPASLAVSLGQRATISCKASQSVDYDGDSYMN |
| light chain | | WYQQKPGQPPKLLIYAASNLESGIPARFSGSGSGTDFTLN |
| | | IHPVEEEDAATYYCQQSNEDPYTFGGGTKLEIK |
| 3C02 VL VH | 232 | MLLLVTSLLLCELPHPAFLLIPDIVMTQSPASLAVSLGQR |
| | | ATISCKASQSVDYDGDSYMNWYQQKPGQPPKLLIYAAS |
| | | NLESGIPARFSGSGSGTDFTLNIHPVEEEDAATYYCQQSN |
| | | EDPYTFGGGTKLEIKGSTSGSGKPGSGEGSTKGQVQLQQ |
| | | SGTVLARPGASVKMSCKASGYSFTNYRMYWVKQRPGQ |
| | | GLEWIGAIYPGNSDTTYKQKFKGKAKLTAVTSASTAYM |
| | | ELSSLTNEDSAVYYCTRGIRGSYFDVWGAGTTVTVSS |
| M1 variable Ligh | 313 | EIVLTQSPATLSLSPGERATISCRASQSVSSNFAWYQQRP |
| Chain | | GQAPRLLIYDASNRATGIPPRFSGSGSGTDFTLTISSLEPE |
| | | DFAAYYCHQRSNWLYTFGQGTKVDIK |
| M1 variable | 314 | QVQLQQSGAEVKKPGASVKVSCKASGYTFTGYYMHWV |
| Heavy Chain | | RQAPGQGLEWMGRINPNSGGTNYAQKFQGRVTMTRDT |
| | | SISTAYMELSRLRSEDTAVYYCARGRYYGMDVWGQGT |
| | | MVTVSS |
| M5 Variable | 315 | DIVMTQSPSSLSASVGDRVTITCRASQSIRYYLSWYQQKP |
| Light chain | | GKAPKLLIYTASILQNGVPSRFSGSGSGTDFTLTISSLQPE |
| | | DFATYYCLQTYTTPDFGPGTKVEIK |
| M5 Variable | 316 | QVQLVQSGAEVEKPGASVKVSCKASGYTFTDYYMHWV |
| Heavy chain | | RQAPGQGLEWMGWINPNSGGTNYAQKFQGRVTMTRDT |
| | | SISTAYMELSRLRSDDTAVYYCASGWDFDYWGQGTLVT |
| | | VSS |
| VD9.V3 | 317 | DIQMTQSPSSLSASVGDRVTITCKSSQSVLYSSNQKNYLA |
| Variable light | | WFQQKPGKAPKLLIYWASTRESGVPSRFSGSGSGTDFTL |
| chain | | TISSLQPEDFATYFCHQYLSSYTFGQGTKVEIK |
| VD9.V3 | 318 | EVQLVESGGGLVQPGGSLRLSCAASGYTFTTYWMHWV |
| Variable Heavy | | RQAPGKGLEWVGYIRPSTGYTEYNQKFKDRFTISADTSK |
| chain | | NTAYLQMNSLRAEDTAVYYCARSRWLLDYWGQGTLVT |
| | | VSS |

In some embodiments, the orientation of the aCAR VH and VL regions is VH-VL. In some embodiments, the orientation of the aCAR VH and VL regions is VL-VH.

In some embodiments, the aCAR scFv comprises a linker that covalently connects the VH and the VL to form the aCAR scFv. In some embodiments, the aCAR comprises a GS based linker sequence, connecting the VH and VL to form the scFv. In some embodiments, the GS linker comprises GGGGS (SEQ ID NO:81). In some embodiments, the aCAR comprises a Whitlow linker sequence, e.g., GSTSGSGKPGSGEGSTKG (SEQ ID NO:82).

10. aCAR Portion: Hinge and Transmembrane Domain

In some embodiments, the bicistronic construct comprises an aCAR portion comprising a hinge transmembrane (TM) domain component. In some embodiments, the aCAR portion comprises a hinge TM domain. In some embodiments, the hinge TM domain comprises a hinge TM domain selected from the group consisting of a CD28 hinge TM domain and a CD8 hinge TM domain (including a CD8a hinge TM domain). In some embodiments, the hinge TM domain is a CD28 hinge TM domain. In some embodiments, the vector comprises a CD8 hinge TM domain. In some embodiments, the vector comprises a CD8a hinge TM domain. In some embodiments, the hinge domain comprises a hinge domain selected from the group consisting of a CD28 hinge domain and a CD8 hinge domain (including a CD8a hinge domain). In some embodiments, the hinge domain is a CD28 hinge domain. In some embodiments, the vector comprises a CD8 hinge domain. In some embodiments, the vector comprises a CD8a hinge domain. In some embodiments, the TM domain comprises a TM domain selected from the group consisting of a CD28 TM domain and a CD8 TM domain (including a CD8a TM domain). In some embodiments, the TM domain is a CD28 TM domain. In some embodiments, the vector comprises a CD8 TM domain. In some embodiments, the vector comprises a CD8a TM domain. In some embodiments, the hinge domain is a CD28 hinge domain of SEQ ID NO:85. In some embodiments, the vector comprises a CD8a hinge domain of SEQ ID NO:84. In some embodiments, the TM domain is a CD28 TM domain of SEQ ID NO:319. In some embodiments, the vector comprises a CD8a TM domain of SEQ ID NO:320.

TABLE 18
aCAR hinge and TM domain sequences

| Sequence | SEQ ID | |
|---|---|---|
| Information | NO | Amino acid sequence |
| CD28 hinge | 85 | IEVMYPPPYLDNEKSNGTIIHVKGKHLCPSP |
| | | LFPGPSKP |
| CD28 TM | 319 | FWVLVVVGGVLACYSLLVTVAFIIFWV |
| CD8alpha hinge | 84 | TTTPAPRPPTPAPTIASQPLSLRPEACRPAAG |
| | | GAVHTRGLDFACD |
| CD8alpha TM | 320 | IYIWAPLAGTCGVLLLSLVITLYC |

11. aCAR Portion: Co-Stimulatory and Activation Signaling Domain

In some embodiments, the bicistronic construct comprises an aCAR portion comprising co-stimulatory domain component. In some embodiments, the aCAR portion comprises a co-stimulatory domain. In some embodiments, the co-stimulatory domain is selected from the group consisting of CD137 (4-1BB) or CD28 or both 4-1BB and CD28 (28BB). In some embodiments, the co-stimulatory domain is a CD137 (4-1BB) co-stimulatory domain. In some embodiments, the co-stimulatory domain is a CD28 co-stimulatory domain. In some embodiments, the activation signaling domain is CD3z domain. In some embodiments, the co-stimulatory domain is a 28BB co-stimulatory domain. In some embodiments, the co-stimulatory domain is 4-1BB (SEQ ID NO:233). In some embodiments, the co-stimulatory domain is CD28 (SEQ ID NO:234). In some embodiments, the activation signaling domain is CD3z (SEQ ID NO:235).

TABLE 19
aCAR co-stimulatory and activation signaling domain sequences

| Sequence | SEQ | |
|---|---|---|
| Information | ID NO | Amino acid sequence |
| 4-1BB costim | 233 | KRGRKKLLYIFKQPFMRPVQTTQEEDGCSCRFPEEEEG |
| | | GCEL |
| CD28 costim | 234 | RSKRSRLLHSDYMNMTPRRPGPTRKHYQPYAPPRDFA |
| | | AY |
| CD3z activation | 235 | RVKFSRSADAPAYQQGQNQLYNELNLGRREEYDVLD |
| signaling | | KRRGRDPEMGGKPRRKNPQEGLYNELQKDKMAEAYS |
| | | EIGMKGERRRGKGHDGLYQGLSTATKDTYDALHMQA |
| | | LPPR |

12. aCAR Portion: Immunoreceptor Tyrosine-Based Activation Motif (ITAM)

In some embodiments, the aCAR portion comprises an Immunoreceptor Tyrosine-Based Activation Motif (ITAM). In some embodiments, the ITAM is a CD3 zeta domain. In some embodiments, the ITAM is a CD3 zeta domain of SEQ ID NO:236. In some embodiments, the ITAM is a CD3 zeta 3F domain of SEQ ID NO:237. In some embodiments, the ITAM is a CD3 zeta 4F domain of SEQ ID NO:238. In some embodiments, the ITAM is a CD3 zeta 4OF domain of SEQ ID NO:239.

TABLE 20
aCAR ITAM domain sequences

| Sequence | SEQ | |
|---|---|---|
| Information | ID NO | Amino acid sequence |
| CD3 zeta domain | 236 | RVKFSRSADAPAYQQGQNQLYNELNLGRREEYDVLD |
| | | KRRGRDPEMGGKPRRKNPQEGLYNELQKDKMAEAY |
| | | SEIGMKGERRRGKGHDGLYQGLSTATKDTYDALHMQ |
| | | ALPPR |
| CD3 Zeta 3F | 237 | RVKFSRSADAPAYQQGQNQLFNELNLGRREEYDVLD |
| | | KRRGRDPEMGGKPRRKNPQEGLYNELQKDKMAEAFS |
| | | EIGMKGERRRGKGHDGLFQGLSTATKDTYDALHMQA |
| | | LPPR |
| CD3 Zeta 4F | 238 | RVKFSRSADAPAYQQGQNQLFNELNLGRREEFDVLD |
| | | KRRGRDPEMGGKPRRKNPQEGLYNELQKDKMAEAFS |
| | | EIGMKGERRRGKGHDGLFQGLSTATKDTYDALHMQA |
| | | LPPR |
| CD3 Zeta 4OF | 239 | RVKFSRSADAPAYQQGQNQLFNELNLGRREEFDVLD |
| | | KRRGRDPEMGGKPRRKNPQEGLFNELQKDKMAEAYS |
| | | EIGMKGERRRGKGHDGLYQGLSTATKDTFDALHMQA |
| | | LPPR |

13. Exemplary aCARs

In some embodiments, the aCAR comprises an scFv component comprising the VL and VH sequences of trastuzumab (SEQ ID NOs: 170 and 171). In some embodiments, the orientation of the aCAR VH and VL regions is VH-VL. In some embodiments, the orientation of the aCAR VH and VL regions is VL-VH. In some embodiments, the aCAR scFv comprises a (G4S)X3 linker (SEQ ID NO:81) linker that covalently connects the VH and the VL to form the aCAR scFv. In some embodiments, the aCAR scFv comprises a Whitlow linker (SEQ ID NO:82) linker that covalently connects the VH and the VL to form the aCAR scFv. In some embodiments, the aCAR comprises a CD8 alpha hinge TM domain (SEQ ID NO:84). In some embodiments, the aCAR comprises a CD28 hinge TM domain (SEQ ID NO:85). In some embodiments, the aCAR comprises a 4-1BB costimulatory domain (SEQ ID NO: 233). In some embodiments, the aCAR comprises a CD28 costimulatory domain (SEQ ID NO: 234). In some embodiments, the aCAR comprises a CD3z activation signaling domain (SEQ ID NO: 235). In some embodiments, the aCAR comprises an ITAM comprising a CD3 zeta domain (SEQ ID NO: 236). In some embodiments, the aCAR comprises an ITAM comprising a CD3 zeta 3F domain (SEQ ID NO: 237). In some embodiments, the aCAR comprises an ITAM comprising a CD3 zeta 4F domain (SEQ ID NO: 238). In some embodiments, the aCAR comprises an ITAM comprising a CD3 zeta 4OF domain (SEQ ID NO: 239). In some embodiments, the aCAR comprises a signal peptide upstream of the aCAR portion, wherein the signal peptide is a CD8 alpha signal peptide (SEQ ID NO: 161), a GM-CSF signal peptide (SEQ ID NO: 162), or a mIgK signal peptide (SEQ ID NO: 306).

In some embodiments, the aCAR comprises an scFv component comprising the VL and VH sequences of trastuzumab F9G (SEQ ID NOs: 307 and 308). In some embodiments, the orientation of the aCAR VH and VL regions is VH-VL. In some embodiments, the orientation of the aCAR VH and VL regions is VL-VH. In some embodiments, the aCAR scFv comprises a (G4S)X3 linker (SEQ ID NO:81) linker that covalently connects the VH and the VL to form the aCAR scFv. In some embodiments, the aCAR scFv comprises a Whitlow linker (SEQ ID NO:82) linker that covalently connects the VH and the VL to form the aCAR scFv. In some embodiments, the aCAR comprises a CD8 alpha hinge TM domain (SEQ ID NO:84). In some embodiments, the aCAR comprises a CD28 hinge TM domain (SEQ ID NO:85). In some embodiments, the aCAR comprises a 4-1BB costimulatory domain (SEQ ID NO: 233). In some embodiments, the aCAR comprises a CD28 costimulatory domain (SEQ ID NO: 234). In some embodiments, the aCAR comprises a CD3z activation signaling domain (SEQ ID NO: 235). In some embodiments, the aCAR comprises an ITAM comprising a CD3 zeta domain (SEQ ID NO: 236). In some embodiments, the aCAR comprises an ITAM comprising a CD3 zeta 3F domain (SEQ ID NO: 237). In some embodiments, the aCAR comprises an ITAM comprising a CD3 zeta 4F domain (SEQ ID NO: 238). In some embodiments, the aCAR comprises an ITAM comprising a CD3 zeta 4OF domain (SEQ ID NO: 239). In some embodiments, the aCAR comprises a signal peptide upstream of the aCAR portion, wherein the signal peptide is a CD8 alpha signal peptide (SEQ ID NO: 161), a GM-CSF signal peptide (SEQ ID NO: 162), or a mIgK signal peptide (SEQ ID NO: 306).

In some embodiments, the aCAR comprises an scFv component comprising the VL and VH sequences of pertuzumab (SEQ ID NOs: 173 and 174). In some embodiments, the orientation of the aCAR VH and VL regions is VH-VL. In some embodiments, the orientation of the aCAR VH and VL regions is VL-VH. In some embodiments, the aCAR scFv comprises a (G4S)X3 linker (SEQ ID NO:81) linker that covalently connects the VH and the VL to form the aCAR scFv. In some embodiments, the aCAR scFv comprises a Whitlow linker (SEQ ID NO:82) linker that covalently connects the VH and the VL to form the aCAR scFv. In some embodiments, the aCAR comprises a CD8 alpha hinge TM domain (SEQ ID NO:84). In some embodiments, the aCAR comprises a CD28 hinge TM domain (SEQ ID NO:85). In some embodiments, the aCAR comprises a 4-1BB costimulatory domain (SEQ ID NO: 233). In some embodiments, the aCAR comprises a CD28 costimulatory domain (SEQ ID NO: 234). In some embodiments, the aCAR comprises a CD3z costimulatory domain (SEQ ID NO: 235). In some embodiments, the aCAR comprises an ITAM comprising a CD3 zeta domain (SEQ ID NO: 236). In some embodiments, the aCAR comprises an ITAM comprising a CD3 zeta 3F domain (SEQ ID NO: 237). In some embodiments, the aCAR comprises an ITAM comprising a CD3 zeta 4F domain (SEQ ID NO: 238). In some embodiments, the aCAR comprises an ITAM comprising a CD3 zeta 4OF domain (SEQ ID NO: 239). In some embodiments, the aCAR comprises a signal peptide upstream of the aCAR portion, wherein the signal peptide is a CD8 alpha signal peptide (SEQ ID NO: 161), a GM-CSF signal peptide (SEQ ID NO: 162), or a mIgK signal peptide (SEQ ID NO: 306).

In some embodiments, the aCAR comprises an scFv component comprising the VL and VH sequences of FRP5 (SEQ ID NOs: 176 and 177). In some embodiments, the orientation of the aCAR VH and VL regions is VH-VL. In some embodiments, the orientation of the aCAR VH and VL regions is VL-VH. In some embodiments, the aCAR scFv comprises a (G4S)X3 linker (SEQ ID NO:81) linker that covalently connects the VH and the VL to form the aCAR scFv. In some embodiments, the aCAR scFv comprises a Whitlow linker (SEQ ID NO:82) linker that covalently connects the VH and the VL to form the aCAR scFv. In some embodiments, the aCAR comprises a CD8 alpha hinge TM domain (SEQ ID NO:84). In some embodiments, the aCAR comprises a CD28 hinge TM domain (SEQ ID NO:85). In some embodiments, the aCAR comprises a 4-1BB costimulatory domain (SEQ ID NO: 233). In some embodiments, the aCAR comprises a CD28 costimulatory domain (SEQ ID NO: 234). In some embodiments, the aCAR comprises a CD3z costimulatory domain (SEQ ID NO: 235). In some embodiments, the aCAR comprises an ITAM comprising a CD3 zeta domain (SEQ ID NO: 236). In some embodiments, the aCAR comprises an ITAM comprising a CD3 zeta 3F domain (SEQ ID NO: 237). In some embodiments, the aCAR comprises an ITAM comprising a CD3 zeta 4F domain (SEQ ID NO: 238). In some embodiments, the aCAR comprises an ITAM comprising a CD3 zeta 4OF domain (SEQ ID NO: 239). In some embodiments, the aCAR comprises a signal peptide upstream of the aCAR portion, wherein the signal peptide is a CD8 alpha signal peptide (SEQ ID NO: 161), a GM-CSF signal peptide (SEQ ID NO: 162), or a mIgK signal peptide (SEQ ID NO: 306).

In some embodiments, the aCAR comprises an scFv component comprising the VL and VH sequences of A21 (SEQ ID NOs: 178 and 179). In some embodiments, the orientation of the aCAR VH and VL regions is VH-VL. In some embodiments, the orientation of the aCAR VH and VL regions is VL-VH. In some embodiments, the aCAR scFv comprises a (G4S)X3 linker (SEQ ID NO:81) linker that covalently connects the VH and the VL to form the aCAR scFv. In some embodiments, the aCAR scFv comprises a Whitlow linker (SEQ ID NO:82) linker that covalently connects the VH and the VL to form the aCAR scFv. In some embodiments, the aCAR comprises a CD8 alpha hinge TM domain (SEQ ID NO:84). In some embodiments, the aCAR comprises a CD28 hinge TM domain (SEQ ID NO:85). In some embodiments, the aCAR comprises a 4-1BB costimulatory domain (SEQ ID NO: 233). In some embodiments, the aCAR comprises a CD28 costimulatory domain (SEQ ID NO: 234). In some embodiments, the aCAR comprises a CD3z costimulatory domain (SEQ ID NO: 235). In some embodiments, the aCAR comprises an ITAM comprising a CD3 zeta domain (SEQ ID NO: 236). In some embodiments, the aCAR comprises an ITAM comprising a CD3 zeta 3F domain (SEQ ID NO: 237). In some embodiments, the aCAR comprises an ITAM comprising a CD3 zeta 4F domain (SEQ ID NO: 238). In some embodiments, the aCAR comprises an ITAM comprising a CD3 zeta 4OF domain (SEQ ID NO: 239). In some embodiments, the aCAR comprises a signal peptide upstream of the aCAR portion, wherein the signal peptide is a CD8 alpha signal peptide (SEQ ID NO: 161), a GM-CSF signal peptide (SEQ ID NO: 162), or a mIgK signal peptide (SEQ ID NO: 306).

In some embodiments, the aCAR comprises an scFv component comprising the VL and VH sequences of XMT1517 (SEQ ID NOs: 180 and 181). In some embodiments, the orientation of the aCAR VH and VL regions is VH-VL. In some embodiments, the orientation of the aCAR VH and VL regions is VL-VH. In some embodiments, the aCAR scFv comprises a (G4S)X3 linker (SEQ ID NO:81) linker that covalently connects the VH and the VL to form the aCAR scFv. In some embodiments, the aCAR scFv comprises a Whitlow linker (SEQ ID NO:82) linker that covalently connects the VH and the VL to form the aCAR scFv. In some embodiments, the aCAR comprises a CD8 alpha hinge TM domain (SEQ ID NO:84). In some embodiments, the aCAR comprises a CD28 hinge TM domain (SEQ ID NO:85). In some embodiments, the aCAR comprises a 4-1BB costimulatory domain (SEQ ID NO: 233). In some embodiments, the aCAR comprises a CD28 costimulatory domain (SEQ ID NO: 234). In some embodiments, the aCAR comprises a CD3z costimulatory domain (SEQ ID NO: 235). In some embodiments, the aCAR comprises an ITAM comprising a CD3 zeta domain (SEQ ID NO: 236). In some embodiments, the aCAR comprises an ITAM comprising a CD3 zeta 3F domain (SEQ ID NO: 237). In some embodiments, the aCAR comprises an ITAM comprising a CD3 zeta 4F domain (SEQ ID NO: 238). In some embodiments, the aCAR comprises an ITAM comprising a CD3 zeta 4OF domain (SEQ ID NO: 239). In some embodiments, the aCAR comprises a signal peptide upstream of the aCAR portion, wherein the signal peptide is a CD8 alpha signal peptide (SEQ ID NO: 161), a GM-CSF signal peptide (SEQ ID NO: 162), or a mIgK signal peptide (SEQ ID NO: 306).

In some embodiments, the aCAR comprises an scFv component comprising the VL and VH sequences of XMT1518 (SEQ ID NOs: 182 and 183). In some embodiments, the orientation of the aCAR VH and VL regions is VH-VL. In some embodiments, the orientation of the aCAR VH and VL regions is VL-VH. In some embodiments, the aCAR scFv comprises a (G4S)X3 linker (SEQ ID NO:81) linker that covalently connects the VH and the VL to form the aCAR scFv. In some embodiments, the aCAR scFv comprises a Whitlow linker (SEQ ID NO:82) linker that covalently connects the VH and the VL to form the aCAR scFv. In some embodiments, the aCAR comprises a CD8 alpha hinge TM domain (SEQ ID NO:84). In some embodiments, the aCAR comprises a CD28 hinge TM domain (SEQ ID NO:85). In some embodiments, the aCAR comprises a 4-1BB costimulatory domain (SEQ ID NO: 233). In some embodiments, the aCAR comprises a CD28 costimulatory domain (SEQ ID NO: 234). In some embodiments, the aCAR comprises a CD3z costimulatory domain (SEQ ID NO: 235). In some embodiments, the aCAR comprises an ITAM comprising a CD3 zeta domain (SEQ ID NO: 236). In some embodiments, the aCAR comprises an ITAM comprising a CD3 zeta 3F domain (SEQ ID NO: 237). In some embodiments, the aCAR comprises an ITAM comprising a CD3 zeta 4F domain (SEQ ID NO: 238). In some embodiments, the aCAR comprises an ITAM comprising a CD3 zeta 4OF domain (SEQ ID NO: 239). In some embodiments, the aCAR comprises a signal peptide upstream of the aCAR portion, wherein the signal peptide is a CD8 alpha signal peptide (SEQ ID NO: 161), a GM-CSF signal peptide (SEQ ID NO: 162), or a mIgK signal peptide (SEQ ID NO: 306).

In some embodiments, the aCAR comprises an scFv component comprising the VL and VH sequences of XMT1519 (SEQ ID NOs: 184 and 185). In some embodiments, the orientation of the aCAR VH and VL regions is VH-VL. In some embodiments, the orientation of the aCAR VH and VL regions is VL-VH. In some embodiments, the aCAR scFv comprises a (G4S)X3 linker (SEQ ID NO:81) linker that covalently connects the VH and the VL to form the aCAR scFv. In some embodiments, the aCAR scFv comprises a Whitlow linker (SEQ ID NO:82) linker that covalently connects the VH and the VL to form the aCAR scFv. In some embodiments, the aCAR comprises a CD8 alpha hinge TM domain (SEQ ID NO:84). In some embodiments, the aCAR comprises a CD28 hinge TM domain (SEQ ID NO:85). In some embodiments, the aCAR comprises a 4-1BB costimulatory domain (SEQ ID NO: 233). In some embodiments, the aCAR comprises a CD28 costimulatory domain (SEQ ID NO: 234). In some embodiments, the aCAR comprises a CD3z costimulatory domain (SEQ ID NO: 235). In some embodiments, the aCAR comprises an ITAM comprising a CD3 zeta domain (SEQ ID NO: 236). In some embodiments, the aCAR comprises an ITAM comprising a CD3 zeta 3F domain (SEQ ID NO: 237). In some embodiments, the aCAR comprises an ITAM comprising a CD3 zeta 4F domain (SEQ ID NO: 238). In some embodiments, the aCAR comprises an ITAM comprising a CD3 zeta 4OF domain (SEQ ID NO: 239). In some embodiments, the aCAR comprises a signal peptide upstream of the aCAR portion, wherein the signal peptide is a CD8 alpha signal peptide (SEQ ID NO: 161), a GM-CSF signal peptide (SEQ ID NO: 162), or a mIgK signal peptide (SEQ ID NO: 306).

In some embodiments, the aCAR comprises an scFv component comprising the VL and VH sequences of FWP51 (SEQ ID NOs: 186 and 187). In some embodiments, the orientation of the aCAR VH and VL regions is VH-VL. In some embodiments, the orientation of the aCAR VH and VL regions is VL-VH. In some embodiments, the aCAR scFv comprises a (G4S)X3 linker (SEQ ID NO:81) linker that covalently connects the VH and the VL to form the aCAR scFv. In some embodiments, the aCAR scFv comprises a Whitlow linker (SEQ ID NO:82) linker that covalently connects the VH and the VL to form the aCAR scFv. In some embodiments, the aCAR comprises a CD8 alpha hinge TM domain (SEQ ID NO:84). In some embodiments, the aCAR comprises a CD28 hinge TM domain (SEQ ID NO:85). In some embodiments, the aCAR comprises a 4-1BB costimulatory domain (SEQ ID NO: 233). In some embodiments, the aCAR comprises a CD28 costimulatory domain (SEQ ID NO: 234). In some embodiments, the aCAR comprises a CD3z costimulatory domain (SEQ ID NO: 235). In some embodiments, the aCAR comprises an ITAM comprising a CD3 zeta domain (SEQ ID NO: 236). In some embodiments, the aCAR comprises an ITAM comprising a CD3 zeta 3F domain (SEQ ID NO: 237). In some embodiments, the aCAR comprises an ITAM comprising a CD3 zeta 4F domain (SEQ ID NO: 238). In some embodiments, the aCAR comprises an ITAM comprising a CD3 zeta 4OF domain (SEQ ID NO: 239). In some embodiments, the aCAR comprises a signal peptide upstream of the aCAR portion, wherein the signal peptide is a CD8 alpha signal peptide (SEQ ID NO: 161), a GM-CSF signal peptide (SEQ ID NO: 162), or a mIgK signal peptide (SEQ ID NO: 306).

In some embodiments, the aCAR comprises an scFv component comprising the anti-HER2 VHH (SEQ ID NO: 309). In some embodiments, the orientation of the aCAR VH and VL regions is VH-VL. In some embodiments, the orientation of the aCAR VH and VL regions is VL-VH. In some embodiments, the aCAR scFv comprises a (G4S)X3 linker (SEQ ID NO:81) linker that covalently connects the VH and the VL to form the aCAR scFv. In some embodiments, the aCAR scFv comprises a Whitlow linker (SEQ ID NO:82) linker that covalently connects the VH and the VL to form the aCAR scFv. In some embodiments, the aCAR comprises a CD8 alpha hinge TM domain (SEQ ID NO:84). In some embodiments, the aCAR comprises a CD28 hinge TM domain (SEQ ID NO:85). In some embodiments, the aCAR comprises a 4-1BB costimulatory domain (SEQ ID NO: 233). In some embodiments, the aCAR comprises a CD28 costimulatory domain (SEQ ID NO: 234). In some embodiments, the aCAR comprises a CD3z costimulatory domain (SEQ ID NO: 235). In some embodiments, the aCAR comprises an ITAM comprising a CD3 zeta domain (SEQ ID NO: 236). In some embodiments, the aCAR comprises an ITAM comprising a CD3 zeta 3F domain (SEQ ID NO: 237). In some embodiments, the aCAR comprises an ITAM comprising a CD3 zeta 4F domain (SEQ ID NO: 238). In some embodiments, the aCAR comprises an ITAM comprising a CD3 zeta 4OF domain (SEQ ID NO: 239).

In some embodiments, the aCAR comprises an scFv component comprising the VL and VH sequences of Cetuximab (SEQ ID NOs: 189 and 190). In some embodiments, the orientation of the aCAR VH and VL regions is VH-VL. In some embodiments, the orientation of the aCAR VH and VL regions is VL-VH. In some embodiments, the aCAR scFv comprises a (G4S)X3 linker (SEQ ID NO:81) linker that covalently connects the VH and the VL to form the aCAR scFv. In some embodiments, the aCAR scFv comprises a Whitlow linker (SEQ ID NO:82) linker that covalently connects the VH and the VL to form the aCAR scFv. In some embodiments, the aCAR comprises a CD8 alpha hinge TM domain (SEQ ID NO:84). In some embodiments, the aCAR comprises a CD28 hinge TM domain (SEQ ID NO:85). In some embodiments, the aCAR comprises a 4-1BB costimulatory domain (SEQ ID NO: 233). In some embodiments, the aCAR comprises a CD28 costimulatory domain (SEQ ID NO: 234). In some embodiments, the aCAR comprises a CD3z costimulatory domain (SEQ ID NO: 235). In some embodiments, the aCAR comprises an ITAM comprising a CD3 zeta domain (SEQ ID NO: 236). In some embodiments, the aCAR comprises an ITAM comprising a CD3 zeta 3F domain (SEQ ID NO: 237). In some embodiments, the aCAR comprises an ITAM comprising a CD3 zeta 4F domain (SEQ ID NO: 238). In some embodiments, the aCAR comprises an ITAM comprising a CD3 zeta 4OF domain (SEQ ID NO: 239). In some embodiments, the aCAR comprises a signal peptide upstream of the aCAR portion, wherein the signal peptide is a CD8 alpha signal peptide (SEQ ID NO: 161), a GM-CSF signal peptide (SEQ ID NO: 162), or a mIgK signal peptide (SEQ ID NO: 306).

In some embodiments, the aCAR comprises an scFv component comprising the VL and VH sequences of Panitumumab (SEQ ID NOs: 192 and 193). In some embodiments, the orientation of the aCAR VH and VL regions is VH-VL. In some embodiments, the orientation of the aCAR VH and VL regions is VL-VH. In some embodiments, the aCAR scFv comprises a (G4S)X3 linker (SEQ ID NO:81) linker that covalently connects the VH and the VL to form the aCAR scFv. In some embodiments, the aCAR scFv comprises a Whitlow linker (SEQ ID NO:82) linker that covalently connects the VH and the VL to form the aCAR scFv. In some embodiments, the aCAR comprises a CD8 alpha hinge TM domain (SEQ ID NO:84). In some embodiments, the aCAR comprises a CD28 hinge TM domain (SEQ ID NO:85). In some embodiments, the aCAR comprises a 4-1BB costimulatory domain (SEQ ID NO: 233). In some embodiments, the aCAR comprises a CD28 costimulatory domain (SEQ ID NO: 234). In some embodiments, the aCAR comprises a CD3z costimulatory domain (SEQ ID NO: 235). In some embodiments, the aCAR comprises an ITAM comprising a CD3 zeta domain (SEQ ID NO: 236). In some embodiments, the aCAR comprises an ITAM comprising a CD3 zeta 3F domain (SEQ ID NO: 237). In some embodiments, the aCAR comprises an ITAM comprising a CD3 zeta 4F domain (SEQ ID NO: 238). In some embodiments, the aCAR comprises an ITAM comprising a CD3 zeta 4OF domain (SEQ ID NO: 239). In some embodiments, the aCAR comprises a signal peptide upstream of the aCAR portion, wherein the signal peptide is a CD8 alpha signal peptide (SEQ ID NO: 161), a GM-CSF signal peptide (SEQ ID NO: 162), or a mIgK signal peptide (SEQ ID NO: 306).

In some embodiments, the aCAR comprises an scFv component comprising the VL and VH sequences of Imgatuzumab (SEQ ID NOs: 195 and 196). In some embodiments, the orientation of the aCAR VH and VL regions is VH-VL. In some embodiments, the orientation of the aCAR VH and VL regions is VL-VH. In some embodiments, the aCAR scFv comprises a (G4S)X3 linker (SEQ ID NO:81) linker that covalently connects the VH and the VL to form the aCAR scFv. In some embodiments, the aCAR scFv comprises a Whitlow linker (SEQ ID NO:82) linker that covalently connects the VH and the VL to form the aCAR scFv. In some embodiments, the aCAR comprises a CD8 alpha hinge TM domain (SEQ ID NO:84). In some embodiments, the aCAR comprises a CD28 hinge TM domain (SEQ ID NO:85). In some embodiments, the aCAR comprises a 4-1BB costimulatory domain (SEQ ID NO: 233). In some embodiments, the aCAR comprises a CD28 costimulatory domain (SEQ ID NO: 234). In some embodiments, the aCAR comprises a CD3z costimulatory domain (SEQ ID NO: 235). In some embodiments, the aCAR comprises an ITAM comprising a CD3 zeta domain (SEQ ID NO: 236). In some embodiments, the aCAR comprises an ITAM comprising a CD3 zeta 3F domain (SEQ ID NO: 237). In some embodiments, the aCAR comprises an ITAM comprising a CD3 zeta 4F domain (SEQ ID NO: 238). In some embodiments, the aCAR comprises an ITAM comprising a CD3 zeta 4OF domain (SEQ ID NO: 239). In some embodiments, the aCAR comprises a signal peptide upstream of the aCAR portion, wherein the signal peptide is a CD8 alpha signal peptide (SEQ ID NO: 161), a GM-CSF signal peptide (SEQ ID NO: 162), or a mIgK signal peptide (SEQ ID NO: 306).

In some embodiments, the aCAR comprises an scFv component comprising the VL and VH sequences of Nimotuzumab (SEQ ID NOs: 197 and 198). In some embodiments, the orientation of the aCAR VH and VL regions is VH-VL. In some embodiments, the orientation of the aCAR VH and VL regions is VL-VH. In some embodiments, the aCAR scFv comprises a (G4S)X3 linker (SEQ ID NO:81) linker that covalently connects the VH and the VL to form the aCAR scFv. In some embodiments, the aCAR scFv comprises a Whitlow linker (SEQ ID NO:82) linker that covalently connects the VH and the VL to form the aCAR scFv. In some embodiments, the aCAR comprises a CD8 alpha hinge TM domain (SEQ ID NO:84). In some embodiments, the aCAR comprises a CD28 hinge TM domain (SEQ ID NO:85). In some embodiments, the aCAR comprises a 4-1BB costimulatory domain (SEQ ID NO: 233). In some embodiments, the aCAR comprises a CD28 costimulatory domain (SEQ ID NO: 234). In some embodiments, the aCAR comprises a CD3z costimulatory domain (SEQ ID NO: 235). In some embodiments, the aCAR comprises an ITAM comprising a CD3 zeta domain (SEQ ID NO: 236). In some embodiments, the aCAR comprises an ITAM comprising a CD3 zeta 3F domain (SEQ ID NO: 237). In some embodiments, the aCAR comprises an ITAM comprising a CD3 zeta 4F domain (SEQ ID NO: 238). In some embodiments, the aCAR comprises an ITAM comprising a CD3 zeta 4OF domain (SEQ ID NO: 239). In some embodiments, the aCAR comprises a signal peptide upstream of the aCAR portion, wherein the signal peptide is a CD8 alpha signal peptide (SEQ ID NO: 161), a GM-CSF signal peptide (SEQ ID NO: 162), or a mIgK signal peptide (SEQ ID NO: 306).

In some embodiments, the aCAR comprises an scFv component comprising the VL and VH sequences of Nimotuzumab (K5) (SEQ ID NOs: 310 and 311). In some embodiments, the orientation of the aCAR VH and VL regions is VH-VL. In some embodiments, the orientation of the aCAR VH and VL regions is VL-VH. In some embodiments, the aCAR scFv comprises a (G4S)X3 linker (SEQ ID NO:81) linker that covalently connects the VH and the VL to form the aCAR scFv. In some embodiments, the aCAR scFv comprises a Whitlow linker (SEQ ID NO:82) linker that covalently connects the VH and the VL to form the aCAR scFv. In some embodiments, the aCAR comprises a CD8 alpha hinge TM domain (SEQ ID NO:84). In some embodiments, the aCAR comprises a CD28 hinge TM domain (SEQ ID NO:85). In some embodiments, the aCAR comprises a 4-1BB costimulatory domain (SEQ ID NO: 233). In some embodiments, the aCAR comprises a CD28 costimulatory domain (SEQ ID NO: 234). In some embodiments, the aCAR comprises a CD3z costimulatory domain (SEQ ID NO: 235). In some embodiments, the aCAR comprises an ITAM comprising a CD3 zeta domain (SEQ ID NO: 236). In some embodiments, the aCAR comprises an ITAM comprising a CD3 zeta 3F domain (SEQ ID NO: 237). In some embodiments, the aCAR comprises an ITAM comprising a CD3 zeta 4F domain (SEQ ID NO: 238). In some embodiments, the aCAR comprises an ITAM comprising a CD3 zeta 4OF domain (SEQ ID NO: 239). In some embodiments, the aCAR comprises a signal peptide upstream of the aCAR portion, wherein the signal peptide is a CD8 alpha signal peptide (SEQ ID NO: 161), a GM-CSF signal peptide (SEQ ID NO: 162), or a mIgK signal peptide (SEQ ID NO: 306).

In some embodiments, the aCAR comprises an scFv component comprising the VL and VH sequences of Necitumumab (SEQ ID NOs: 199 and 200). In some embodiments, the orientation of the aCAR VH and VL regions is VH-VL. In some embodiments, the orientation of the aCAR VH and VL regions is VL-VH. In some embodiments, the aCAR scFv comprises a (G4S)X3 linker (SEQ ID NO:81) linker that covalently connects the VH and the VL to form the aCAR scFv. In some embodiments, the aCAR scFv comprises a Whitlow linker (SEQ ID NO:82) linker that covalently connects the VH and the VL to form the aCAR scFv. In some embodiments, the aCAR comprises a CD8 alpha hinge TM domain (SEQ ID NO:84). In some embodiments, the aCAR comprises a CD28 hinge TM domain (SEQ ID NO:85). In some embodiments, the aCAR comprises a 4-1BB costimulatory domain (SEQ ID NO: 233). In some embodiments, the aCAR comprises a CD28 costimulatory domain (SEQ ID NO: 234). In some embodiments, the aCAR comprises a CD3z costimulatory domain (SEQ ID NO: 235). In some embodiments, the aCAR comprises an ITAM comprising a CD3 zeta domain (SEQ ID NO: 236). In some embodiments, the aCAR comprises an ITAM comprising a CD3 zeta 3F domain (SEQ ID NO: 237). In some embodiments, the aCAR comprises an ITAM comprising a CD3 zeta 4F domain (SEQ ID NO: 238). In some embodiments, the aCAR comprises an ITAM comprising a CD3 zeta 4OF domain (SEQ ID NO: 239). In some embodiments, the aCAR comprises a signal peptide upstream of the aCAR portion, wherein the signal peptide is a CD8 alpha signal peptide (SEQ ID NO: 161), a GM-CSF signal peptide (SEQ ID NO: 162), or a mIgK signal peptide (SEQ ID NO: 306).

In some embodiments, the aCAR comprises an scFv component comprising the VL and VH sequences of ICR62 (SEQ ID NOs: 201 and 202). In some embodiments, the orientation of the aCAR VH and VL regions is VH-VL. In some embodiments, the orientation of the aCAR VH and VL regions is VL-VH. In some embodiments, the aCAR scFv comprises a (G4S)X3 linker (SEQ ID NO:81) linker that covalently connects the VH and the VL to form the aCAR scFv. In some embodiments, the aCAR scFv comprises a Whitlow linker (SEQ ID NO:82) linker that covalently connects the VH and the VL to form the aCAR scFv. In some embodiments, the aCAR comprises a CD8 alpha hinge TM domain (SEQ ID NO:84). In some embodiments, the aCAR comprises a CD28 hinge TM domain (SEQ ID NO:85). In some embodiments, the aCAR comprises a 4-1BB costimulatory domain (SEQ ID NO: 233). In some embodiments, the aCAR comprises a CD28 costimulatory domain (SEQ ID NO: 234). In some embodiments, the aCAR comprises a CD3z costimulatory domain (SEQ ID NO: 235). In some embodiments, the aCAR comprises an ITAM comprising a CD3 zeta domain (SEQ ID NO: 236). In some embodiments, the aCAR comprises an ITAM comprising a CD3 zeta 3F domain (SEQ ID NO: 237). In some embodiments, the aCAR comprises an ITAM comprising a CD3 zeta 4F domain (SEQ ID NO: 238). In some embodiments, the aCAR comprises an ITAM comprising a CD3 zeta 4OF domain (SEQ ID NO: 239). In some embodiments, the aCAR comprises a signal peptide upstream of the aCAR portion, wherein the signal peptide is a CD8 alpha signal peptide (SEQ ID NO: 161), a GM-CSF signal peptide (SEQ ID NO: 162), or a mIgK signal peptide (SEQ ID NO: 306).

In some embodiments, the aCAR comprises an scFv component comprising the VL and VH sequences of Matuzumab (SEQ ID NOs: 204 and 205). In some embodiments, the orientation of the aCAR VH and VL regions is VH-VL. In some embodiments, the orientation of the aCAR VH and VL regions is VL-VH. In some embodiments, the aCAR scFv comprises a (G4S)X3 linker (SEQ ID NO:81) linker that covalently connects the VH and the VL to form the aCAR scFv. In some embodiments, the aCAR scFv comprises a Whitlow linker (SEQ ID NO:82) linker that covalently connects the VH and the VL to form the aCAR scFv. In some embodiments, the aCAR comprises a CD8 alpha hinge TM domain (SEQ ID NO:84). In some embodiments, the aCAR comprises a CD28 hinge TM domain (SEQ ID NO:85). In some embodiments, the aCAR comprises a 4-1BB costimulatory domain (SEQ ID NO: 233). In some embodiments, the aCAR comprises a CD28 costimulatory domain (SEQ ID NO: 234). In some embodiments, the aCAR comprises a CD3z costimulatory domain (SEQ ID NO: 235). In some embodiments, the aCAR comprises an ITAM comprising a CD3 zeta domain (SEQ ID NO: 236). In some embodiments, the aCAR comprises an ITAM comprising a CD3 zeta 3F domain (SEQ ID NO: 237). In some embodiments, the aCAR comprises an ITAM comprising a CD3 zeta 4F domain (SEQ ID NO: 238). In some embodiments, the aCAR comprises an ITAM comprising a CD3 zeta 4OF domain (SEQ ID NO: 239). In some embodiments, the aCAR comprises a signal peptide upstream of the aCAR portion, wherein the signal peptide is a CD8 alpha signal peptide (SEQ ID NO: 161), a GM-CSF signal peptide (SEQ ID NO: 162), or a mIgK signal peptide (SEQ ID NO: 306).

In some embodiments, the aCAR comprises an scFv component comprising the VL and VH sequences of C10 (SEQ ID NOs: 206 and 207). In some embodiments, the orientation of the aCAR VH and VL regions is VH-VL. In some embodiments, the orientation of the aCAR VH and VL regions is VL-VH. In some embodiments, the aCAR scFv comprises a (G4S)X3 linker (SEQ ID NO:81) linker that covalently connects the VH and the VL to form the aCAR scFv. In some embodiments, the aCAR scFv comprises a Whitlow linker (SEQ ID NO:82) linker that covalently connects the VH and the VL to form the aCAR scFv. In some embodiments, the aCAR comprises a CD8 alpha hinge TM domain (SEQ ID NO:84). In some embodiments, the aCAR comprises a CD28 hinge TM domain (SEQ ID NO:85). In some embodiments, the aCAR comprises a 4-1BB costimulatory domain (SEQ ID NO: 233). In some embodiments, the aCAR comprises a CD28 costimulatory domain (SEQ ID NO: 234). In some embodiments, the aCAR comprises a CD3z costimulatory domain (SEQ ID NO: 235). In some embodiments, the aCAR comprises an ITAM comprising a CD3 zeta domain (SEQ ID NO: 236). In some embodiments, the aCAR comprises an ITAM comprising a CD3 zeta 3F domain (SEQ ID NO: 237). In some embodiments, the aCAR comprises an ITAM comprising a CD3 zeta 4F domain (SEQ ID NO: 238). In some embodiments, the aCAR comprises an ITAM comprising a CD3 zeta 4OF domain (SEQ ID NO: 239). In some embodiments, the aCAR comprises a signal peptide upstream of the aCAR portion, wherein the signal peptide is a CD8 alpha signal peptide (SEQ ID NO: 161), a GM-CSF signal peptide (SEQ ID NO: 162), or a mIgK signal peptide (SEQ ID NO: 306).

In some embodiments, the aCAR comprises an scFv component comprising the VL and VH sequences of Zalutumumab (SEQ ID NOs: 208 and 209). In some embodiments, the orientation of the aCAR VH and VL regions is VH-VL. In some embodiments, the orientation of the aCAR VH and VL regions is VL-VH. In some embodiments, the aCAR scFv comprises a (G4S)X3 linker (SEQ ID NO:81) linker that covalently connects the VH and the VL to form the aCAR scFv. In some embodiments, the aCAR scFv comprises a Whitlow linker (SEQ ID NO:82) linker that covalently connects the VH and the VL to form the aCAR scFv. In some embodiments, the aCAR comprises a CD8 alpha hinge TM domain (SEQ ID NO:84). In some embodiments, the aCAR comprises a CD28 hinge TM domain (SEQ ID NO:85). In some embodiments, the aCAR comprises a 4-1BB costimulatory domain (SEQ ID NO: 233). In some embodiments, the aCAR comprises a CD28 costimulatory domain (SEQ ID NO: 234). In some embodiments, the aCAR comprises a CD3z costimulatory domain (SEQ ID NO: 235). In some embodiments, the aCAR comprises an ITAM comprising a CD3 zeta domain (SEQ ID NO: 236). In some embodiments, the aCAR comprises an ITAM comprising a CD3 zeta 3F domain (SEQ ID NO: 237). In some embodiments, the aCAR comprises an ITAM comprising a CD3 zeta 4F domain (SEQ ID NO: 238). In some embodiments, the aCAR comprises an ITAM comprising a CD3 zeta 4OF domain (SEQ ID NO: 239). In some embodiments, the aCAR comprises a signal peptide upstream of the aCAR portion, wherein the signal peptide is a CD8 alpha signal peptide (SEQ ID NO: 161), a GM-CSF signal peptide (SEQ ID NO: 162), or a mIgK signal peptide (SEQ ID NO: 306).

In some embodiments, the aCAR comprises an scFv component comprising the VL and VH sequences of P1X (SEQ ID NOs: 210 and 211). In some embodiments, the orientation of the aCAR VH and VL regions is VH-VL. In some embodiments, the orientation of the aCAR VH and VL regions is VL-VH. In some embodiments, the aCAR scFv comprises a (G4S)X3 linker (SEQ ID NO:81) linker that covalently connects the VH and the VL to form the aCAR scFv. In some embodiments, the aCAR scFv comprises a Whitlow linker (SEQ ID NO:82) linker that covalently connects the VH and the VL to form the aCAR scFv. In some embodiments, the aCAR comprises a CD8 alpha hinge TM domain (SEQ ID NO:84). In some embodiments, the aCAR comprises a CD28 hinge TM domain (SEQ ID NO:85). In some embodiments, the aCAR comprises a 4-1BB costimulatory domain (SEQ ID NO: 233). In some embodiments, the aCAR comprises a CD28 costimulatory domain (SEQ ID NO: 234). In some embodiments, the aCAR comprises a CD3z costimulatory domain (SEQ ID NO: 235). In some embodiments, the aCAR comprises an ITAM comprising a CD3 zeta domain (SEQ ID NO: 236). In some embodiments, the aCAR comprises an ITAM comprising a CD3 zeta 3F domain (SEQ ID NO: 237). In some embodiments, the aCAR comprises an ITAM comprising a CD3 zeta 4F domain (SEQ ID NO: 238). In some embodiments, the aCAR comprises an ITAM comprising a CD3 zeta 4OF domain (SEQ ID NO: 239). In some embodiments, the aCAR comprises a signal peptide upstream of the aCAR portion, wherein the signal peptide is a CD8 alpha signal peptide (SEQ ID NO: 161), a GM-CSF signal peptide (SEQ ID NO: 162), or a mIgK signal peptide (SEQ ID NO: 306).

In some embodiments, the aCAR comprises an scFv component comprising the VL and VH sequences of P2X (SEQ ID NOs: 212 and 213). In some embodiments, the orientation of the aCAR VH and VL regions is VH-VL. In some embodiments, the orientation of the aCAR VH and VL regions is VL-VH. In some embodiments, the aCAR scFv comprises a (G4S)X3 linker (SEQ ID NO:81) linker that covalently connects the VH and the VL to form the aCAR scFv. In some embodiments, the aCAR scFv comprises a Whitlow linker (SEQ ID NO:82) linker that covalently connects the VH and the VL to form the aCAR scFv. In some embodiments, the aCAR comprises a CD8 alpha hinge TM domain (SEQ ID NO:84). In some embodiments, the aCAR comprises a CD28 hinge TM domain (SEQ ID NO:85). In some embodiments, the aCAR comprises a 4-1BB costimulatory domain (SEQ ID NO: 233). In some embodiments, the aCAR comprises a CD28 costimulatory domain (SEQ ID NO: 234). In some embodiments, the aCAR comprises a CD3z costimulatory domain (SEQ ID NO: 235). In some embodiments, the aCAR comprises an ITAM comprising a CD3 zeta domain (SEQ ID NO: 236). In some embodiments, the aCAR comprises an ITAM comprising a CD3 zeta 3F domain (SEQ ID NO: 237). In some embodiments, the aCAR comprises an ITAM comprising a CD3 zeta 4F domain (SEQ ID NO: 238). In some embodiments, the aCAR comprises an ITAM comprising a CD3 zeta 4OF domain (SEQ ID NO: 239). In some embodiments, the aCAR comprises a signal peptide upstream of the aCAR portion, wherein the signal peptide is a CD8 alpha signal peptide (SEQ ID NO: 161), a GM-CSF signal peptide (SEQ ID NO: 162), or a mIgK signal peptide (SEQ ID NO: 306).

In some embodiments, the aCAR comprises an scFv component comprising the VL and VH sequences of P3X (SEQ ID NOs: 214 and 215). In some embodiments, the orientation of the aCAR VH and VL regions is VH-VL. In some embodiments, the orientation of the aCAR VH and VL regions is VL-VH. In some embodiments, the aCAR scFv comprises a (G4S)X3 linker (SEQ ID NO:81) linker that covalently connects the VH and the VL to form the aCAR scFv. In some embodiments, the aCAR scFv comprises a Whitlow linker (SEQ ID NO:82) linker that covalently connects the VH and the VL to form the aCAR scFv. In some embodiments, the aCAR comprises a CD8 alpha hinge TM domain (SEQ ID NO:84). In some embodiments, the aCAR comprises a CD28 hinge TM domain (SEQ ID NO:85). In some embodiments, the aCAR comprises a 4-1BB costimulatory domain (SEQ ID NO: 233). In some embodiments, the aCAR comprises a CD28 costimulatory domain (SEQ ID NO: 234). In some embodiments, the aCAR comprises a CD3z costimulatory domain (SEQ ID NO: 235). In some embodiments, the aCAR comprises an ITAM comprising a CD3 zeta domain (SEQ ID NO: 236). In some embodiments, the aCAR comprises an ITAM comprising a CD3 zeta 3F domain (SEQ ID NO: 237). In some embodiments, the aCAR comprises an ITAM comprising a CD3 zeta 4F domain (SEQ ID NO: 238). In some embodiments, the aCAR comprises an ITAM comprising a CD3 zeta 4OF domain (SEQ ID NO: 239). In some embodiments, the aCAR comprises a signal peptide upstream of the aCAR portion, wherein the signal peptide is a CD8 alpha signal peptide (SEQ ID NO: 161), a GM-CSF signal peptide (SEQ ID NO: 162), or a mIgK signal peptide (SEQ ID NO: 306).

In some embodiments, the aCAR comprises an scFv component comprising the VHH sequence of EGFR-1a1-VHH (SEQ ID NO: 216). In some embodiments, the orientation of the aCAR VH and VL regions is VH-VL. In some embodiments, the orientation of the aCAR VH and VL regions is VL-VH. In some embodiments, the aCAR scFv comprises a (G4S)X3 linker (SEQ ID NO:81) linker that covalently connects the VH and the VL to form the aCAR scFv. In some embodiments, the aCAR scFv comprises a Whitlow linker (SEQ ID NO:82) linker that covalently connects the VH and the VL to form the aCAR scFv. In some embodiments, the aCAR comprises a CD8 alpha hinge TM domain (SEQ ID NO:84). In some embodiments, the aCAR comprises a CD28 hinge TM domain (SEQ ID NO:85). In some embodiments, the aCAR comprises a 4-1BB costimulatory domain (SEQ ID NO: 233). In some embodiments, the aCAR comprises a CD28 costimulatory domain (SEQ ID NO: 234). In some embodiments, the aCAR comprises a CD3z costimulatory domain (SEQ ID NO: 235). In some embodiments, the aCAR comprises an ITAM comprising a CD3 zeta domain (SEQ ID NO: 236). In some embodiments, the aCAR comprises an ITAM comprising a CD3 zeta 3F domain (SEQ ID NO: 237). In some embodiments, the aCAR comprises an ITAM comprising a CD3 zeta 4F domain (SEQ ID NO: 238). In some embodiments, the aCAR comprises an ITAM comprising a CD3 zeta 4OF domain (SEQ ID NO: 239). In some embodiments, the aCAR comprises a signal peptide upstream of the aCAR portion, wherein the signal peptide is a CD8 alpha signal peptide (SEQ ID NO: 161), a GM-CSF signal peptide (SEQ ID NO: 162), or a mIgK signal peptide (SEQ ID NO: 306).

In some embodiments, the aCAR comprises an scFv component comprising the VHH sequence of EGFR-VHH (SEQ ID NO: 312). In some embodiments, the orientation of the aCAR VH and VL regions is VH-VL. In some embodiments, the orientation of the aCAR VH and VL regions is VL-VH. In some embodiments, the aCAR scFv comprises a (G4S)X3 linker (SEQ ID NO:81) linker that covalently connects the VH and the VL to form the aCAR scFv. In some embodiments, the aCAR scFv comprises a Whitlow linker (SEQ ID NO:82) linker that covalently connects the VH and the VL to form the aCAR scFv. In some embodiments, the aCAR comprises a CD8 alpha hinge TM domain (SEQ ID NO:84). In some embodiments, the aCAR comprises a CD28 hinge TM domain (SEQ ID NO:85). In some embodiments, the aCAR comprises a 4-1BB costimulatory domain (SEQ ID NO: 233). In some embodiments, the aCAR comprises a CD28 costimulatory domain (SEQ ID NO: 234). In some embodiments, the aCAR comprises a CD3z costimulatory domain (SEQ ID NO: 235). In some embodiments, the aCAR comprises an ITAM comprising a CD3 zeta domain (SEQ ID NO: 236). In some embodiments, the aCAR comprises an ITAM comprising a CD3 zeta 3F domain (SEQ ID NO: 237). In some embodiments, the aCAR comprises an ITAM comprising a CD3 zeta 4F domain (SEQ ID NO: 238). In some embodiments, the aCAR comprises an ITAM comprising a CD3 zeta 4OF domain (SEQ ID NO: 239). In some embodiments, the aCAR comprises a signal peptide upstream of the aCAR portion, wherein the signal peptide is a CD8 alpha signal peptide (SEQ ID NO: 161), a GM-CSF signal peptide (SEQ ID NO: 162), or a mIgK signal peptide (SEQ ID NO: 306).

In some embodiments, the aCAR comprises an scFv component comprising the VL and VH sequences of Amatuximab (SEQ ID NOs: 217 and 218). In some embodiments, the orientation of the aCAR VH and VL regions is VH-VL. In some embodiments, the orientation of the aCAR VH and VL regions is VL-VH. In some embodiments, the aCAR scFv comprises a (G4S)X3 linker (SEQ ID NO:81) linker that covalently connects the VH and the VL to form the aCAR scFv. In some embodiments, the aCAR scFv comprises a Whitlow linker (SEQ ID NO:82) linker that covalently connects the VH and the VL to form the aCAR scFv. In some embodiments, the aCAR comprises a CD8 alpha hinge TM domain (SEQ ID NO:84). In some embodiments, the aCAR comprises a CD28 hinge TM domain (SEQ ID NO:85). In some embodiments, the aCAR comprises a 4-1BB costimulatory domain (SEQ ID NO: 233). In some embodiments, the aCAR comprises a CD28 costimulatory domain (SEQ ID NO: 234). In some embodiments, the aCAR comprises a CD3z costimulatory domain (SEQ ID NO: 235). In some embodiments, the aCAR comprises an ITAM comprising a CD3 zeta domain (SEQ ID NO: 236). In some embodiments, the aCAR comprises an ITAM comprising a CD3 zeta 3F domain (SEQ ID NO: 237). In some embodiments, the aCAR comprises an ITAM comprising a CD3 zeta 4F domain (SEQ ID NO: 238). In some embodiments, the aCAR comprises an ITAM comprising a CD3 zeta 4OF domain (SEQ ID NO: 239). In some embodiments, the aCAR comprises a signal peptide upstream of the aCAR portion, wherein the signal peptide is a CD8 alpha signal peptide (SEQ ID NO: 161), a GM-CSF signal peptide (SEQ ID NO: 162), or a mIgK signal peptide (SEQ ID NO: 306).

In some embodiments, the aCAR comprises an scFv component comprising the VL and VH sequences of P4 (SEQ ID NOs: 219 and 220). In some embodiments, the orientation of the aCAR VH and VL regions is VH-VL. In some embodiments, the orientation of the aCAR VH and VL regions is VL-VH. In some embodiments, the aCAR scFv comprises a (G4S)X3 linker (SEQ ID NO:81) linker that covalently connects the VH and the VL to form the aCAR scFv. In some embodiments, the aCAR scFv comprises a Whitlow linker (SEQ ID NO:82) linker that covalently connects the VH and the VL to form the aCAR scFv. In some embodiments, the aCAR comprises a CD8 alpha hinge TM domain (SEQ ID NO:84). In some embodiments, the aCAR comprises a CD28 hinge TM domain (SEQ ID NO:85). In some embodiments, the aCAR comprises a 4-1BB costimulatory domain (SEQ ID NO: 233). In some embodiments, the aCAR comprises a CD28 costimulatory domain (SEQ ID NO: 234). In some embodiments, the aCAR comprises a CD3z costimulatory domain (SEQ ID NO: 235). In some embodiments, the aCAR comprises an ITAM comprising a CD3 zeta domain (SEQ ID NO: 236). In some embodiments, the aCAR comprises an ITAM comprising a CD3 zeta 3F domain (SEQ ID NO: 237). In some embodiments, the aCAR comprises an ITAM comprising a CD3 zeta 4F domain (SEQ ID NO: 238). In some embodiments, the aCAR comprises an ITAM comprising a CD3 zeta 4OF domain (SEQ ID NO: 239). In some embodiments, the aCAR comprises a signal peptide upstream of the aCAR portion, wherein the signal peptide is a CD8 alpha signal peptide (SEQ ID NO: 161), a GM-CSF signal peptide (SEQ ID NO: 162), or a mIgK signal peptide (SEQ ID NO: 306).

In some embodiments, the aCAR comprises an scFv component comprising the VL and VH sequences of SS1 (SEQ ID NOs: 222 and 223). In some embodiments, the orientation of the aCAR VH and VL regions is VH-VL. In some embodiments, the orientation of the aCAR VH and VL regions is VL-VH. In some embodiments, the aCAR scFv comprises a (G4S)X3 linker (SEQ ID NO:81) linker that covalently connects the VH and the VL to form the aCAR scFv. In some embodiments, the aCAR scFv comprises a Whitlow linker (SEQ ID NO:82) linker that covalently connects the VH and the VL to form the aCAR scFv. In some embodiments, the aCAR comprises a CD8 alpha hinge TM domain (SEQ ID NO:84). In some embodiments, the aCAR comprises a CD28 hinge TM domain (SEQ ID NO:85). In some embodiments, the aCAR comprises a 4-1BB costimulatory domain (SEQ ID NO: 233). In some embodiments, the aCAR comprises a CD28 costimulatory domain (SEQ ID NO: 234). In some embodiments, the aCAR comprises a CD3z costimulatory domain (SEQ ID NO: 235). In some embodiments, the aCAR comprises an ITAM comprising a CD3 zeta domain (SEQ ID NO: 236). In some embodiments, the aCAR comprises an ITAM comprising a CD3 zeta 3F domain (SEQ ID NO: 237). In some embodiments, the aCAR comprises an ITAM comprising a CD3 zeta 4F domain (SEQ ID NO: 238). In some embodiments, the aCAR comprises an ITAM comprising a CD3 zeta 4OF domain (SEQ ID NO: 239). In some embodiments, the aCAR comprises a signal peptide upstream of the aCAR portion, wherein the signal peptide is a CD8 alpha signal peptide (SEQ ID NO: 161), a GM-CSF signal peptide (SEQ ID NO: 162), or a mIgK signal peptide (SEQ ID NO: 306).

In some embodiments, the aCAR comprises an scFv component comprising the VHH sequence of SD1 (SEQ ID NO: 225). In some embodiments, the orientation of the aCAR VH and VL regions is VH-VL. In some embodiments, the orientation of the aCAR VH and VL regions is VL-VH. In some embodiments, the aCAR scFv comprises a (G4S)X3 linker (SEQ ID NO:81) linker that covalently connects the VH and the VL to form the aCAR scFv. In some embodiments, the aCAR scFv comprises a Whitlow linker (SEQ ID NO:82) linker that covalently connects the VH and the VL to form the aCAR scFv. In some embodiments, the aCAR comprises a CD8 alpha hinge TM domain (SEQ ID NO:84). In some embodiments, the aCAR comprises a CD28 hinge TM domain (SEQ ID NO:85). In some embodiments, the aCAR comprises a 4-1BB costimulatory domain (SEQ ID NO: 233). In some embodiments, the aCAR comprises a CD28 costimulatory domain (SEQ ID NO: 234). In some embodiments, the aCAR comprises a CD3z costimulatory domain (SEQ ID NO: 235). In some embodiments, the aCAR comprises an ITAM comprising a CD3 zeta domain (SEQ ID NO: 236). In some embodiments, the aCAR comprises an ITAM comprising a CD3 zeta 3F domain (SEQ ID NO: 237). In some embodiments, the aCAR comprises an ITAM comprising a CD3 zeta 4F domain (SEQ ID NO: 238). In some embodiments, the aCAR comprises an ITAM comprising a CD3 zeta 4OF domain (SEQ ID NO: 239). In some embodiments, the aCAR comprises a signal peptide upstream of the aCAR portion, wherein the signal peptide is a CD8 alpha signal peptide (SEQ ID NO: 161), a GM-CSF signal peptide (SEQ ID NO: 162), or a mIgK signal peptide (SEQ ID NO: 306).

In some embodiments, the aCAR comprises an scFv component comprising the VHH sequence of SD2 (SEQ ID NO: 226). In some embodiments, the orientation of the aCAR VH and VL regions is VH-VL. In some embodiments, the orientation of the aCAR VH and VL regions is VL-VH. In some embodiments, the aCAR scFv comprises a (G4S)X3 linker (SEQ ID NO:81) linker that covalently connects the VH and the VL to form the aCAR scFv. In some embodiments, the aCAR scFv comprises a Whitlow linker (SEQ ID NO:82) linker that covalently connects the VH and the VL to form the aCAR scFv. In some embodiments, the aCAR comprises a CD8 alpha hinge TM domain (SEQ ID NO:84). In some embodiments, the aCAR comprises a CD28 hinge TM domain (SEQ ID NO:85). In some embodiments, the aCAR comprises a 4-1BB costimulatory domain (SEQ ID NO: 233). In some embodiments, the aCAR comprises a CD28 costimulatory domain (SEQ ID NO: 234). In some embodiments, the aCAR comprises a CD3z costimulatory domain (SEQ ID NO: 235). In some embodiments, the aCAR comprises an ITAM comprising a CD3 zeta domain (SEQ ID NO: 236). In some embodiments, the aCAR comprises an ITAM comprising a CD3 zeta 3F domain (SEQ ID NO: 237). In some embodiments, the aCAR comprises an ITAM comprising a CD3 zeta 4F domain (SEQ ID NO: 238). In some embodiments, the aCAR comprises an ITAM comprising a CD3 zeta 4OF domain (SEQ ID NO: 239). In some embodiments, the aCAR comprises a signal peptide upstream of the aCAR portion, wherein the signal peptide is a CD8 alpha signal peptide (SEQ ID NO: 161), a GM-CSF signal peptide (SEQ ID NO: 162), or a mIgK signal peptide (SEQ ID NO: 306).

In some embodiments, the aCAR comprises an scFv component comprising the VL and VH sequences of 1H7 (SEQ ID NOs: 227 and 228). In some embodiments, the orientation of the aCAR VH and VL regions is VH-VL. In some embodiments, the orientation of the aCAR VH and VL regions is VL-VH. In some embodiments, the aCAR scFv comprises a (G4S)X3 linker (SEQ ID NO:81) linker that covalently connects the VH and the VL to form the aCAR scFv. In some embodiments, the aCAR scFv comprises a Whitlow linker (SEQ ID NO:82) linker that covalently connects the VH and the VL to form the aCAR scFv. In some embodiments, the aCAR comprises a CD8 alpha hinge TM domain (SEQ ID NO:84). In some embodiments, the aCAR comprises a CD28 hinge TM domain (SEQ ID NO:85). In some embodiments, the aCAR comprises a 4-1BB costimulatory domain (SEQ ID NO: 233). In some embodiments, the aCAR comprises a CD28 costimulatory domain (SEQ ID NO: 234). In some embodiments, the aCAR comprises a CD3z costimulatory domain (SEQ ID NO: 235). In some embodiments, the aCAR comprises an ITAM comprising a CD3 zeta domain (SEQ ID NO: 236). In some embodiments, the aCAR comprises an ITAM comprising a CD3 zeta 3F domain (SEQ ID NO: 237). In some embodiments, the aCAR comprises an ITAM comprising a CD3 zeta 4F domain (SEQ ID NO: 238). In some embodiments, the aCAR comprises an ITAM comprising a CD3 zeta 4OF domain (SEQ ID NO: 239). In some embodiments, the aCAR comprises a signal peptide upstream of the aCAR portion, wherein the signal peptide is a CD8 alpha signal peptide (SEQ ID NO: 161), a GM-CSF signal peptide (SEQ ID NO: 162), or a mIgK signal peptide (SEQ ID NO: 306).

In some embodiments, the aCAR comprises an scFv component comprising the VL and VH sequences of 3C02 (SEQ ID NOs: 230 and 231). In some embodiments, the orientation of the aCAR VH and VL regions is VH-VL. In some embodiments, the orientation of the aCAR VH and VL regions is VL-VH. In some embodiments, the aCAR scFv comprises a (G4S)X3 linker (SEQ ID NO:81) linker that covalently connects the VH and the VL to form the aCAR scFv. In some embodiments, the aCAR scFv comprises a Whitlow linker (SEQ ID NO:82) linker that covalently connects the VH and the VL to form the aCAR scFv. In some embodiments, the aCAR comprises a CD8 alpha hinge TM domain (SEQ ID NO:84). In some embodiments, the aCAR comprises a CD28 hinge TM domain (SEQ ID NO:85). In some embodiments, the aCAR comprises a 4-1BB costimulatory domain (SEQ ID NO: 233). In some embodiments, the aCAR comprises a CD28 costimulatory domain (SEQ ID NO: 234). In some embodiments, the aCAR comprises a CD3z costimulatory domain (SEQ ID NO: 235). In some embodiments, the aCAR comprises an ITAM comprising a CD3 zeta domain (SEQ ID NO: 236). In some embodiments, the aCAR comprises an ITAM comprising a CD3 zeta 3F domain (SEQ ID NO: 237). In some embodiments, the aCAR comprises an ITAM comprising a CD3 zeta 4F domain (SEQ ID NO: 238). In some embodiments, the aCAR comprises an ITAM comprising a CD3 zeta 4OF domain (SEQ ID NO: 239). In some embodiments, the aCAR comprises a signal peptide upstream of the aCAR portion, wherein the signal peptide is a CD8 alpha signal peptide (SEQ ID NO: 161), a GM-CSF signal peptide (SEQ ID NO: 162), or a mIgK signal peptide (SEQ ID NO: 306).

In some embodiments, the aCAR has a set of components shown in Table 21.

TABLE 21
aCAR constructs

| | | | | | | | |
|---|---|---|---|---|---|---|---|
| | Signal | | scFv | | | Co- | |
| Construct | Peptide | scFv | Linker | Hinge | TM | stimulatory | Signaling |

Anti-EGFR

| | | | | | | | |
|---|---|---|---|---|---|---|---|
| MC0001 | CD8 | Imgatuzumab | Whitlow | CD8 | CD8 | 4-1BB | CD3 zeta |
| (VR1) | alpha | VL_VH | | alpha | alpha | | |
| MC0002 | CD8 | Cextuximab | Whitlow | CD8 | CD8 | 4-1BB | CD3 zeta |
| (VR2) | alpha | VL_VH | | alpha | alpha | | |
| MC0003 | CD8 | Panitumumab | Whitlow | CD8 | CD8 | 4-1BB | CD3 zeta |
| (VR3) | alpha | VL_VH | | alpha | alpha | | |
| MC0004 | CD8 | Nimotuzumab | Whitlow | CD8 | CD8 | 4-1BB | CD3 zeta |
| (VR4) | alpha | VL_VH | | alpha | alpha | | |
| MC0005 | CD8 | Necitumumab | Whitlow | CD8 | CD8 | 4-1BB | CD3 zeta |
| (VR5) | alpha | VL_VH | | alpha | alpha | | |
| MC0163 | GM- | ICR62 VH | Whitlow | CD8 | CD8 | 4-1BB | CD3 zeta |
| (VR163) | CSF | VL | | alpha | alpha | | |
| MC0164 | GM- | ICR62 VL | Whitlow | CD8 | CD8 | 4-1BB | CD3 zeta |
| (VR164) | CSF | VH | | alpha | alpha | | |
| MC0165 | GM- | Matuzumab | Whitlow | CD8 | CD8 | 4-1BB | CD3 zeta |
| (VR165) | CSF | VH VL BBz | | alpha | alpha | | |
| MC0166 | GM- | Matuzumab | Whitlow | CD8 | CD8 | 4-1BB | CD3 zeta |
| (VR166) | CSF | VL VH BBz | | alpha | alpha | | |
| MC0167 | GM- | C10 VH VL | Whitlow | CD8 | CD8 | 4-1BB | CD3 zeta |
| (VR167) | CSF | BBz | | alpha | alpha | | |
| MC0168 | GM- | C10 VL VH | Whitlow | CD8 | CD8 | 4-1BB | CD3 zeta |
| (VR168) | CSF | BBz | | alpha | alpha | | |
| MC0169 | GM- | Zalutumumab | Whitlow | CD8 | CD8 | 4-1BB | CD3 zeta |
| (VR169) | CSF | VH VL BBz | | alpha | alpha | | |
| MC0170 | GM- | Zalutumumab | Whitlow | CD8 | CD8 | 4-1BB | CD3 zeta |
| (VR170) | CSF | VL VH BBz | | alpha | alpha | | |
| MC0171 | GM- | P1X VH VL | Whitlow | CD8 | CD8 | 4-1BB | CD3 zeta |
| (VR171) | CSF | BBz | | alpha | alpha | | |
| MC0172 | GM- | P1X VL VH | Whitlow | CD8 | CD8 | 4-1BB | CD3 zeta |
| (VR172) | CSF | BBz | | alpha | alpha | | |
| MC0173 | GM- | P2X VH VL | Whitlow | CD8 | CD8 | 4-1BB | CD3 zeta |
| (VR173) | CSF | BBz | | alpha | alpha | | |
| MC0174 | GM- | P2X VL VH | Whitlow | CD8 | CD8 | 4-1BB | CD3 zeta |
| (VR174) | CSF | BBz | | alpha | alpha | | |
| MC0175 | GM- | P3X VH VL | Whitlow | CD8 | CD8 | 4-1BB | CD3 zeta |
| (VR175) | CSF | BBz | | alpha | alpha | | |
| MC0176 | GM- | P3X VL VH | Whitlow | CD8 | CD8 | 4-1BB | CD3 zeta |
| (VR176) | CSF | BBz | | alpha | alpha | | |
| MC0177 | GM- | EGFR-la1- | Whitlow | CD8 | CD8 | 4-1BB | CD3 zeta |
| (VR177) | CSF | VHH BBz | | alpha | alpha | | |
| N/A | CD8 | ICR62 | Whitlow | CD8 | CD8 | 4-1BB | CD3 zeta |
| | alpha | VH_VL | | alpha | alpha | | |
| N/A | CD8 | ICR62 | Whitlow | CD8 | CD8 | 4-1BB | CD3 zeta |
| | alpha | VL_VH | | alpha | alpha | | |
| N/A | CD8 | Matuzumab | Whitlow | CD8 | CD8 | 4-1BB | CD3 zeta |
| | alpha | VH_VL | | alpha | alpha | | |
| N/A | CD8 | Matuzumab | Whitlow | CD8 | CD8 | 4-1BB | CD3 zeta |
| | alpha | VL_VH | | alpha | alpha | | |
| N/A | CD8 | C10 VH VL | Whitlow | CD8 | CD8 | 4-1BB | CD3 zeta |
| | alpha | BBz | | alpha | alpha | | |
| N/A | CD8 | C10 VL VH | Whitlow | CD8 | CD8 | 4-1BB | CD3 zeta |
| | alpha | BBz | | alpha | alpha | | |
| N/A | CD8 | Zalutumumab | whitlow | CD8 | CD8 | 4-1BB | CD3 zeta |
| | alpha | VH VL | | alpha | alpha | | |
| MC0483 | CD8 | Zalutumumab | whitlow | CD8 | CD8 | 4-1BB | CD3 zeta |
| (VR483) | alpha | VL_VH | | alpha | alpha | | |
| N/A | CD8 | P1X VH VL | Whitlow | CD8 | CD8 | 4-1BB | CD3 zeta |
| | alpha | BBz | | alpha | alpha | | |
| N/A | CD8 | P1X VL VH | Whitlow | CD8 | CD8 | 4-1BB | CD3 zeta |
| | alpha | BBz | | alpha | alpha | | |
| N/A | CD8 | P2X VH VL | Whitlow | CD8 | CD8 | 4-1BB | CD3 zeta |
| | alpha | BBz | | alpha | alpha | | |
| N/A | CD8 | P2X VL VH | Whitlow | CD8 | CD8 | 4-1BB | CD3 zeta |
| | alpha | BBz | | alpha | alpha | | |
| N/A | CD8 | P3X VH VL | Whitlow | CD8 | CD8 | 4-1BB | CD3 zeta |
| | alpha | BBz | | alpha | alpha | | |
| N/A | CD8 | P3X VL VH | Whitlow | CD8 | CD8 | 4-1BB | CD3 zeta |
| | alpha | BBz | | alpha | alpha | | |
| MC0484 | CD8 | EGFR-l1a- | whitlow | CD8 | CD8 | 4-1BB | CD3 zeta |
| (VR484) | alpha | VHH | | alpha | alpha | | |

Anti-HER2

| | | | | | | | |
|---|---|---|---|---|---|---|---|
| MC0006 | CD8 | Trastuzumab | Whitlow | CD8 | CD8 | 4-1BB | CD3 zeta |
| (VR6) | alpha | VL_VH | | alpha | alpha | | |
| MC0007 | CD8 | Pertuzumab | Whitlow | CD8 | CD8 | 4-1BB | CD3 zeta |
| (VR7) | alpha | VL_VH | | alpha | alpha | | |
| MC0008 | CD8 | FRP5 VL VH | Whitlow | CD8 | CD8 | 4-1BB | CD3 zeta |
| (VR8) | alpha | | | alpha | alpha | | |
| MC0009 | CD8 | A21 VL VH | Whitlow | CD8 | CD8 | 4-1BB | CD3 zeta |
| (VR9) | alpha | | | alpha | alpha | | |
| MC0178 | GM- | XMT1517 | Whitlow | CD8 | CD8 | 4-1BB | CD3 zeta |
| (VR178) | CSF | VH VL | | alpha | alpha | | |
| MC0179 | GM- | XMT1517 | Whitlow | CD8 | CD8 | 4-1BB | CD3 zeta |
| (VR179) | CSF | VL VH | | alpha | alpha | | |
| MC0180 | GM- | XMT1518 | Whitlow | CD8 | CD8 | 4-1BB | CD3 zeta |
| (VR180) | CSF | VH VL | | alpha | alpha | | |
| MC0181 | GM- | XMT1518 | Whitlow | CD8 | CD8 | 4-1BB | CD3 zeta |
| (VR181) | CSF | VL VH | | alpha | alpha | | |
| MC0182 | GM- | XMT1519 | Whitlow | CD8 | CD8 | 4-1BB | CD3 zeta |
| (VR182) | CSF | VH VL | | alpha | alpha | | |
| MC0183 | GM- | XMT1519 | Whitlow | CD8 | CD8 | 4-1BB | CD3 zeta |
| (VR183) | CSF | VL VH | | alpha | alpha | | |
| MC0184 | GM- | FWP51 | Whitlow | CD8 | CD8 | 4-1BB | CD3 zeta |
| (VR184) | CSF | VH VL | | alpha | alpha | | |
| MC0185 | GM- | FWP51 | Whitlow | CD8 | CD8 | 4-1BB | CD3 zeta |
| (VR185) | CSF | VL VH | | alpha | alpha | | |
| N/A | GM- | Trastuzumab | Whitlow | CD8 | CD8 | 4-1BB | CD3 zeta |
| | CSF | VL_VH | | alpha | alpha | | |
| N/A | GM- | Pertuzumab | Whitlow | CD8 | CD8 | 4-1BB | CD3 zeta |
| | CSF | VL_VH | | alpha | alpha | | |
| N/A | GM- | FRP5 VL VH | Whitlow | CD8 | CD8 | 4-1BB | CD3 zeta |
| | CSF | | | alpha | alpha | | |
| N/A | GM- | A21 VL VH | Whitlow | CD8 | CD8 | 4-1BB | CD3 zeta |
| | CSF | | | alpha | alpha | | |
| N/A | CD8 | XMT1517 | Whitlow | CD8 | CD8 | 4-1BB | CD3 zeta |
| | alpha | VH VL | | alpha | alpha | | |
| N/A | CD8 | XMT1517 | Whitlow | CD8 | CD8 | 4-1BB | CD3 zeta |
| | alpha | VL VH | | alpha | alpha | | |
| N/A | CD8 | XMT1518 | Whitlow | CD8 | CD8 | 4-1BB | CD3 zeta |
| | alpha | VH VL | | alpha | alpha | | |
| N/A | CD8 | XMT1518 | Whitlow | CD8 | CD8 | 4-1BB | CD3 zeta |
| | alpha | VL VH | | alpha | alpha | | |
| N/A | CD8 | XMT1519 | Whitlow | CD8 | CD8 | 4-1BB | CD3 zeta |
| | alpha | VH VL | | alpha | alpha | | |
| N/A | CD8 | XMT1519 | Whitlow | CD8 | CD8 | 4-1BB | CD3 zeta |
| | alpha | VL VH | | alpha | alpha | | |
| N/A | CD8 | FWP51 | Whitlow | CD8 | CD8 | 4-1BB | CD3 zeta |
| | alpha | VH VL | | alpha | alpha | | |
| N/A | CD8 | FWP51 | Whitlow | CD8 | CD8 | 4-1BB | CD3 zeta |
| | alpha | VL VH | | alpha | alpha | | |

Anti-Mesothelin

| | | | | | | | |
|---|---|---|---|---|---|---|---|
| MC0159 | GM- | Amatuximab | Whitlow | CD8 | CD8 | 4-1BB | CD3 zeta |
| (VR159) | CSF | VH VL | | alpha | alpha | | |
| MC0160 | GM- | Amatuximab | Whitlow | CD8 | CD8 | 4-1BB | CD3 zeta |
| (VR160) | CSF | VL VL | | alpha | alpha | | |
| MC0161 | GM- | P4 VH VL | Whitlow | CD8 | CD8 | 4-1BB | CD3 zeta |
| (VR161) | CSF | | | alpha | alpha | | |
| MC0162 | GM- | P4 VL VH | Whitlow | CD8 | CD8 | 4-1BB | CD3 zeta |
| (VR162) | CSF | | | alpha | alpha | | |
| MC0186 | GM- | SS1 VH VL | Whitlow | CD8 | CD8 | 4-1BB | CD3 zeta |
| (VR186) | CSF | | | alpha | alpha | | |
| MC0187 | GM- | SS1 VL VH | Whitlow | CD8 | CD8 | 4-1BB | CD3 zeta |
| (VR187) | CSF | | | alpha | alpha | | |
| MC0188 | GM- | SD1 VHH | Whitlow | CD8 | CD8 | 4-1BB | CD3 zeta |
| (VR188) | CSF | | | alpha | alpha | | |
| MC0189 | GM- | SD2 VHH | Whitlow | CD8 | CD8 | 4-1BB | CD3 zeta |
| (VR189) | CSF | | | alpha | alpha | | |
| MC0190 | GM- | 1H07 VH VL | Whitlow | CD8 | CD8 | 4-1BB | CD3 zeta |
| (VR190) | CSF | | | alpha | alpha | | |
| MC0191 | GM- | 1H07 VL VH | Whitlow | CD8 | CD8 | 4-1BB | CD3 zeta |
| (VR191) | CSF | | | alpha | alpha | | |
| MC0192 | GM- | 3C02 VH VL | Whitlow | CD8 | CD8 | 4-1BB | CD3 zeta |
| (VR192) | CSF | | | alpha | alpha | | |
| MC0193 | GM- | 3C02 VL VH | Whitlow | CD8 | CD8 | 4-1BB | CD3 zeta |
| (VR193) | CSF | | | alpha | alpha | | |
| N/A | CD8 | Amatuximab | Whitlow | CD8 | CD8 | 4-1BB | CD3 zeta |
| | alpha | VH VL | | alpha | alpha | | |
| MC0485 | CD8 | Amatuximab | Whitlow | CD8 | CD8 | 4-1BB | CD3 zeta |
| (VR485) | alpha | VL VH | | alpha | alpha | | |
| N/A | CD8 | P4 VH VL | Whitlow | CD8 | CD8 | 4-1BB | CD3 zeta |
| | alpha | | | alpha | alpha | | |
| MC0487 | CD8 | P4 VL VH | Whitlow | CD8 | CD8 | 4-1BB | CD3 zeta |
| (VR487) | alpha | | | alpha | alpha | | |
| N/A | CD8 | SS1 VH VL | Whitlow | CD8 | CD8 | 4-1BB | CD3 zeta |
| | alpha | | | alpha | alpha | | |
| MC0488 | CD8 | SS1 VL VH | Whitlow | CD8 | CD8 | 4-1BB | CD3 zeta |
| (VR488) | alpha | | | alpha | alpha | | |
| N/A | CD8 | SD1 VHH | Whitlow | CD8 | CD8 | 4-1BB | CD3 zeta |
| | alpha | | | alpha | alpha | | |
| N/A | CD8 | SD2 VHH | Whitlow | CD8 | CD8 | 4-1BB | CD3 zeta |
| | alpha | | | alpha | alpha | | |
| N/A | CD8 | 1H07 VH VL | Whitlow | CD8 | CD8 | 4-1BB | CD3 zeta |
| | alpha | | | alpha | alpha | | |
| MC0490 | CD8 | 1H07 VL VH | Whitlow | CD8 | CD8 | 4-1BB | CD3 zeta |
| (VR490) | alpha | | | alpha | alpha | | |
| N/A | CD8 | 3C02 VH VL | Whitlow | CD8 | CD8 | 4-1BB | CD3 zeta |
| | alpha | | | alpha | alpha | | |
| N/A | CD8 | 3C02 VL VH | Whitlow | CD8 | CD8 | 4-1BB | CD3 zeta |
| | alpha | | | alpha | alpha | | |
| MC0486 | CD8 | M1 VL VH | Whitlow | CD8 | CD8 | 4-1BB | CD3 zeta |
| (VR486) | alpha | | | alpha | alpha | | |
| MC0498 | CD8 | M5 VL VH | Whitlow | CD8 | CD8 | 4-1BB | CD3 zeta |
| (VR498) | alpha | | | alpha | alpha | | |
| MC0489 | CD8 | 7D9.V3 VL | Whitlow | CD8 | CD8 | 4-1BB | CD3 zeta |
| (VR489) | alpha | VH | | alpha | alpha | | |

14. Optional shRNA

In some embodiments, the bicistronic construct comprises an optional short hairpin RNA (shRNA). In some embodiments, the bicistronic construct comprises an HLA-A2 shRNA. In some embodiments, the bicistronic construct comprises an HLA-A2 shRNA having a sequence of SEQ ID NO:240. In some embodiments, the bicistronic construct comprises an HLA-A2 shRNA having a sequence of SEQ ID NO:241. In some embodiments, the bicistronic construct comprises an HLA-beta2 shRNA. In some embodiments, the bicistronic construct comprises an HLA-beta2 shRNA having a sequence of SEQ ID NO:242. In some embodiments, the bicistronic construct comprises an HLA-A2 shRNA having both sequences of SEQ ID NO:240 and SEQ ID NO:242. In some embodiments, the bicistronic construct comprises an HLA-A2 shRNA having both sequences of SEQ ID NO:241 and SEQ ID NO:242.

TABLE 22
shRNA sequences

| Sequence | SEQ ID | |
|---|---|---|
| Information | NO | Nucleotide Sequence |
| HLA-A2-shRNA | 240 | GGATTACATCGCCCTGAAAGTTCAAGAGACTTTCAGGGC |
| 1 | | GATGTAATCCTTTTTT |
| HLA-A2-shRNA | 241 | CACCTGCCATGTGCAGCATGATTTGTGTAGTCATGCTGC |
| 2 | | ACATGGCAGGTG |
| HLA-beta2- | 242 | GAATGGAGAGAGAATTGAATTCAAGAGATTCAATTCTCT |
| shRNA | | CTCCATTC |

### 15. Monocistronic Constructs

In some embodiments, the iCAR and aCAR constructs are expressed by separate vectors, and the iCAR/aCAR pairs are co-expressed in cells. Methods of co-expressing multiple constructs in the same cell are well known in the art and include, e.g., co-transfection of two or more expression vectors, integration of the constructs into the same or different loci within a cell, optionally followed by enrichment for co-expression.

### III. Car-T Bicistronic iCAR/aCAR Vector Construction

In some embodiments, the bicistronic construct or co-transduction of monocistronic aCAR and iCAR constructs allows for the iCAR and the aCAR to be encoded by a single nucleic acid vector. In some embodiments, the present invention provides a vector comprising a nucleic acid molecule of the invention as defined in any one of the above embodiments, and at least one control element, such as a promoter, operably linked to the nucleic acid molecule.

In some embodiments, the vector is a lentiviral (LV) vector. In some embodiments, the LV vector is a commercially available LV vector. In some embodiments, the LV vector includes but is not limited to pLenti, pLVX-Puro, pLVX-IRES-Puro/Neo/Hygro, pLVx-EFla-IRES (TAKARA), and/or pcLV-EFla (Sirion). In some embodiments, the LV vector is pLVX-Puro. In some embodiments, the LV vector is pLVX-IRES-Puro/Neo/Hygro. In some embodiments, the LV vector is pLVx-EF1a-IRES (TAKARA). In some embodiments, the LV vector is pcLV-EF1a (Sirion).

In some embodiments, the vector comprises an EFl promoter. In some embodiments, the vector comprises a CMV promoter. In some embodiments, the vector comprises a PGK promoter.

In some embodiments, the nucleotide sequence of the vector comprises an internal ribosome entry site (IRES) between the nucleotide sequence encoding for the aCAR and the nucleotide sequence encoding for the iCAR. In general, the nucleotide sequence encoding for the aCAR and the nucleotide sequence encoding for the iCAR can be in any sequential order, but in particular embodiments, the nucleotide sequence encoding for the aCAR is downstream of the nucleotide sequence encoding for the iCAR.

In some embodiments, the nucleotide sequences encoding for the aCAR and the iCAR are encoded on a single vector. In some embodiments, the vector comprises an internal ribosome entry site (IRES) between the nucleotide sequence encoding for the aCAR and the nucleotide sequence encoding for the iCAR. In some embodiments, the nucleotide sequence encoding for the aCAR is downstream of the nucleotide sequence encoding for the iCAR. In some embodiments, the nucleotide sequence comprises a viral self-cleaving 2A peptide located between the nucleotide sequence encoding for the aCAR and the nucleotide sequence encoding for the iCAR. In some embodiments, the nucleotide sequence of the vector comprises a viral self-cleaving 2A peptide between the nucleotide sequence encoding for the aCAR and the nucleotide sequence encoding for the iCAR. In some embodiments, the viral self-cleaving 2A peptide includes is the T2A from Thosea asigna virus (TaV). In some embodiments, the vector comprises a nucleotide sequence encoding the constitutive aCAR linked via a flexible linker to said iCAR.

The immune cells may be transfected with the appropriate nucleic acid molecule described herein by e.g., RNA transfection or by incorporation in a plasmid fit for replication and/or transcription in a eukaryotic cell or a viral vector. In some embodiments, the vector is selected from a retroviral or lentiviral vector.

Combinations of retroviral vector and an appropriate packaging line can also be used, where the capsid proteins will be functional for infecting human cells. Several amphotropic virus-producing cell lines are known, including PA12 (Miller, et al. (1985) Mol. Cell. Biol. 5:431-437); PA317 (Miller, et al. (1986) Mol. Cell. Bioi. 6:2895-2902); and CRIP (Danos, et ai. (1988) Proc. Nati. Acad. Sci. USA 85:6460-6464). Alternatively, non-amphotropic particles can be used, such as, particles pseudotyped with VSVG, RD 114 or GAL V envelope and in some embodiments produced in a PG13 cell line. Cells can further be transduced by direct co-culture with producer cells, e.g., by the method of Bregni, et ai. (1992) Blood 80: 1418-1422, or culturing with viral supernatant alone or concentrated vector stocks, e.g., by the method of Xu, et ai. (1994) Exp. Hemat. 22:223-230; and Hughes, et ai. (1992) J Clin. Invest. 89: 1817.

In some embodiments, the iCAR and aCAR are encoded by different constructs, for example as separate monocistronic aCAR and iCAR constructs. In some embodiments, the iCAR and aCAR are encoded by a single construct, for example as separate monocistronic aCAR and iCAR constructs within a single expression vector.

In some embodiments, the iCAR and aCAR are encoded by the same expression vector. In some embodiments, the expression vector comprises a nucleic acid sequence that encodes a bicistronic iCAR/aCAR selected from the group consisting of SEQ ID NO:1, SEQ ID NO:3, SEQ ID NO:5, SEQ ID NO:7, SEQ ID NO:9, SEQ ID NO:11, SEQ ID NO:13, SEQ ID NO:15, SEQ ID NO:17, SEQ ID NO:19, SEQ ID NO:21, SEQ ID NO:23, SEQ ID NO:25, SEQ ID NO:27, SEQ ID NO:29, SEQ ID NO:31, SEQ ID NO:33, SEQ ID NO:35, SEQ ID NO:275, SEQ ID NO:277, SEQ ID NO:279, SEQ ID NO:281, SEQ ID NO:321, SEQ ID NO:323, and SEQ ID NO:325.

In some embodiments, the expression vector comprises a bicistronic iCAR/aCAR nucleic acid sequence selected from the group consisting of SEQ ID NO:1, SEQ ID NO:3, SEQ ID NO:5, SEQ ID NO:7, SEQ ID NO:9, SEQ ID NO:11, SEQ ID NO:13, SEQ ID NO:15, SEQ ID NO:17, SEQ ID NO:19, SEQ ID NO:21, SEQ ID NO:23, SEQ ID NO:25, SEQ ID NO:27, SEQ ID NO:29, SEQ ID NO:31, SEQ ID NO:33, SEQ ID NO:35, SEQ ID NO:275, SEQ ID NO:277, SEQ ID NO:279, SEQ ID NO:281, SEQ ID NO:321, SEQ ID NO:323, and SEQ ID NO:325.

In some embodiments, the expression vector comprises a bicistronic iCAR/aCAR nucleic acid that encodes an amino acid sequence selected from the group consisting of SEQ ID NO:2, SEQ ID NO:4, SEQ ID NO:6, SEQ ID NO:8, SEQ ID NO:10, SEQ ID NO:12, SEQ ID NO:14, SEQ ID NO:16, SEQ ID NO:18, SEQ ID NO:20, SEQ ID NO:22, SEQ ID NO:24, SEQ ID NO:26, SEQ ID NO:28, SEQ ID NO:30, SEQ ID NO:32, SEQ ID NO:34, SEQ ID NO:36, SEQ ID NO:276, SEQ ID NO:278, SEQ ID NO:280, SEQ ID NO:282, SEQ ID NO:322, SEQ ID NO:324, and SEQ ID NO:326.

In some embodiments, the nucleic acid sequence that encodes a bicistronic iCAR/aCAR exhibits 75%, 80%, 85%, 88%, 90%, 91%, 92%, 93%, 94%, 95%, 96%, 97%, 98%, 99% or 100% sequence identity to a nucleic acid sequence selected from the group consisting of SEQ ID NO:1, SEQ ID NO:3, SEQ ID NO:5, SEQ ID NO:7, SEQ ID NO:9, SEQ ID NO:11, SEQ ID NO:13, SEQ ID NO:15, SEQ ID NO:17, SEQ ID NO:19, SEQ ID NO:21, SEQ ID NO:23, SEQ ID NO:25, SEQ ID NO:27, SEQ ID NO:29, SEQ ID NO:31, SEQ ID NO:33, SEQ ID NO:35, SEQ ID NO:275, SEQ ID NO:277, SEQ ID NO:279, SEQ ID NO:281, SEQ ID NO:321, SEQ ID NO:323, and SEQ ID NO:325.

In some embodiments, the nucleic acid sequence that encodes a bicistronic iCAR/aCAR exhibits at least 75% sequence identity to a nucleic acid sequence selected from the group consisting of SEQ ID NO:1, SEQ ID NO:3, SEQ ID NO:5, SEQ ID NO:7, SEQ ID NO:9, SEQ ID NO:11, SEQ ID NO:13, SEQ ID NO:15, SEQ ID NO:17, SEQ ID NO:19, SEQ ID NO:21, SEQ ID NO:23, SEQ ID NO:25, SEQ ID NO:27, SEQ ID NO:29, SEQ ID NO:31, SEQ ID NO:33, SEQ ID NO:35, SEQ ID NO:275, SEQ ID NO:277, SEQ ID NO:279, SEQ ID NO:281, SEQ ID NO:321, SEQ ID NO:323, and SEQ ID NO:325.

In some embodiments, the nucleic acid sequence that encodes a bicistronic iCAR/aCAR exhibits at least 80% sequence identity to a nucleic acid sequence selected from the group consisting of SEQ ID NO:1, SEQ ID NO:3, SEQ ID NO:5, SEQ ID NO:7, SEQ ID NO:9, SEQ ID NO:11, SEQ ID NO:13, SEQ ID NO:15, SEQ ID NO:17, SEQ ID NO:19, SEQ ID NO:21, SEQ ID NO:23, SEQ ID NO:25, SEQ ID NO:27, SEQ ID NO:29, SEQ ID NO:31, SEQ ID NO:33, SEQ ID NO:35, SEQ ID NO:275, SEQ ID NO:277, SEQ ID NO:279, SEQ ID NO:281, SEQ ID NO:321, SEQ ID NO:323, and SEQ ID NO:325.

In some embodiments, the nucleic acid sequence that encodes a bicistronic iCAR/aCAR exhibits at least 85% sequence identity to a nucleic acid sequence selected from the group consisting of SEQ ID NO:1, SEQ ID NO:3, SEQ ID NO:5, SEQ ID NO:7, SEQ ID NO:9, SEQ ID NO:11, SEQ ID NO:13, SEQ ID NO:15, SEQ ID NO:17, SEQ ID NO:19, SEQ ID NO:21, SEQ ID NO:23, SEQ ID NO:25, SEQ ID NO:27, SEQ ID NO:29, SEQ ID NO:31, SEQ ID NO:33, SEQ ID NO:35, SEQ ID NO:275, SEQ ID NO:277, SEQ ID NO:279, SEQ ID NO:281, SEQ ID NO:321, SEQ ID NO:323, and SEQ ID NO:325.

In some embodiments, the nucleic acid sequence that encodes a bicistronic iCAR/aCAR exhibits at least 90% sequence identity to a nucleic acid sequence selected from the group consisting of SEQ ID NO:1, SEQ ID NO:3, SEQ ID NO:5, SEQ ID NO:7, SEQ ID NO:9, SEQ ID NO:11, SEQ ID NO:13, SEQ ID NO:15, SEQ ID NO:17, SEQ ID NO:19, SEQ ID NO:21, SEQ ID NO:23, SEQ ID NO:25, SEQ ID NO:27, SEQ ID NO:29, SEQ ID NO:31, SEQ ID NO:33, SEQ ID NO:35, SEQ ID NO:275, SEQ ID NO:277, SEQ ID NO:279, SEQ ID NO:281, SEQ ID NO:321, SEQ ID NO:323, and SEQ ID NO:325.

In some embodiments, the nucleic acid sequence that encodes a bicistronic iCAR/aCAR exhibits at least 91% sequence identity to a nucleic acid sequence selected from the group consisting of SEQ ID NO:1, SEQ ID NO:3, SEQ ID NO:5, SEQ ID NO:7, SEQ ID NO:9, SEQ ID NO:11, SEQ ID NO:13, SEQ ID NO:15, SEQ ID NO:17, SEQ ID NO:19, SEQ ID NO:21, SEQ ID NO:23, SEQ ID NO:25, SEQ ID NO:27, SEQ ID NO:29, SEQ ID NO:31, SEQ ID NO:33, SEQ ID NO:35, SEQ ID NO:275, SEQ ID NO:277, SEQ ID NO:279, SEQ ID NO:281, SEQ ID NO:321, SEQ ID NO:323, and SEQ ID NO:325.

In some embodiments, the nucleic acid sequence that encodes a bicistronic iCAR/aCAR exhibits at least 92% sequence identity to a nucleic acid sequence selected from the group consisting of SEQ ID NO:1, SEQ ID NO:3, SEQ ID NO:5, SEQ ID NO:7, SEQ ID NO:9, SEQ ID NO:11, SEQ ID NO:13, SEQ ID NO:15, SEQ ID NO:17, SEQ ID NO:19, SEQ ID NO:21, SEQ ID NO:23, SEQ ID NO:25, SEQ ID NO:27, SEQ ID NO:29, SEQ ID NO:31, SEQ ID NO:33, SEQ ID NO:35, SEQ ID NO:275, SEQ ID NO:277, SEQ ID NO:279, SEQ ID NO:281, SEQ ID NO:321, SEQ ID NO:323, and SEQ ID NO:325.

In some embodiments, the nucleic acid sequence that encodes a bicistronic iCAR/aCAR exhibits at least 93% sequence identity to a nucleic acid sequence selected from the group consisting of SEQ ID NO:1, SEQ ID NO:3, SEQ ID NO:5, SEQ ID NO:7, SEQ ID NO:9, SEQ ID NO:11, SEQ ID NO:13, SEQ ID NO:15, SEQ ID NO:17, SEQ ID NO:19, SEQ ID NO:21, SEQ ID NO:23, SEQ ID NO:25, SEQ ID NO:27, SEQ ID NO:29, SEQ ID NO:31, SEQ ID NO:33, SEQ ID NO:35, SEQ ID NO:275, SEQ ID NO:277, SEQ ID NO:279, SEQ ID NO:281, SEQ ID NO:321, SEQ ID NO:323, and SEQ ID NO:325.

In some embodiments, the nucleic acid sequence that encodes a bicistronic iCAR/aCAR exhibits at least 94% sequence identity to a nucleic acid sequence selected from the group consisting of SEQ ID NO:1, SEQ ID NO:3, SEQ ID NO:5, SEQ ID NO:7, SEQ ID NO:9, SEQ ID NO:11, SEQ ID NO:13, SEQ ID NO:15, SEQ ID NO:17, SEQ ID NO:19, SEQ ID NO:21, SEQ ID NO:23, SEQ ID NO:25, SEQ ID NO:27, SEQ ID NO:29, SEQ ID NO:31, SEQ ID NO:33, SEQ ID NO:35, SEQ ID NO:275, SEQ ID NO:277, SEQ ID NO:279, SEQ ID NO:281, SEQ ID NO:321, SEQ ID NO:323, and SEQ ID NO:325.

In some embodiments, the nucleic acid sequence that encodes a bicistronic iCAR/aCAR exhibits at least 95% sequence identity to a nucleic acid sequence selected from the group consisting of SEQ ID NO:1, SEQ ID NO:3, SEQ ID NO:5, SEQ ID NO:7, SEQ ID NO:9, SEQ ID NO:11, SEQ ID NO:13, SEQ ID NO:15, SEQ ID NO:17, SEQ ID NO:19, SEQ ID NO:21, SEQ ID NO:23, SEQ ID NO:25, SEQ ID NO:27, SEQ ID NO:29, SEQ ID NO:31, SEQ ID NO:33, SEQ ID NO:35, SEQ ID NO:275, SEQ ID NO:277, SEQ ID NO:279, SEQ ID NO:281, SEQ ID NO:321, SEQ ID NO:323, and SEQ ID NO:325.

In some embodiments, the nucleic acid sequence that encodes a bicistronic iCAR/aCAR exhibits at least 96% sequence identity to a nucleic acid sequence selected from the group consisting of SEQ ID NO:1, SEQ ID NO:3, SEQ ID NO:5, SEQ ID NO:7, SEQ ID NO:9, SEQ ID NO:11, SEQ ID NO:13, SEQ ID NO:15, SEQ ID NO:17, SEQ ID NO:19, SEQ ID NO:21, SEQ ID NO:23, SEQ ID NO:25, SEQ ID NO:27, SEQ ID NO:29, SEQ ID NO:31, SEQ ID NO:33, SEQ ID NO:35, SEQ ID NO:275, SEQ ID NO:277, SEQ ID NO:279, SEQ ID NO:281, SEQ ID NO:321, SEQ ID NO:323, and SEQ ID NO:325.

In some embodiments, the nucleic acid sequence that encodes a bicistronic iCAR/aCAR exhibits at least 97% sequence identity to a nucleic acid sequence selected from the group consisting of SEQ ID NO:1, SEQ ID NO:3, SEQ ID NO:5, SEQ ID NO:7, SEQ ID NO:9, SEQ ID NO:11, SEQ ID NO:13, SEQ ID NO:15, SEQ ID NO:17, SEQ ID NO:19, SEQ ID NO:21, SEQ ID NO:23, SEQ ID NO:25, SEQ ID NO:27, SEQ ID NO:29, SEQ ID NO:31, SEQ ID NO:33, SEQ ID NO:35, SEQ ID NO:275, SEQ ID NO:277, SEQ ID NO:279, SEQ ID NO:281, SEQ ID NO:321, SEQ ID NO:323, and SEQ ID NO:325.

In some embodiments, the nucleic acid sequence that encodes a bicistronic iCAR/aCAR exhibits at least 98% sequence identity to a nucleic acid sequence selected from the group consisting of SEQ ID NO:1, SEQ ID NO:3, SEQ ID NO:5, SEQ ID NO:7, SEQ ID NO:9, SEQ ID NO:11, SEQ ID NO:13, SEQ ID NO:15, SEQ ID NO:17, SEQ ID NO:19, SEQ ID NO:21, SEQ ID NO:23, SEQ ID NO:25, SEQ ID NO:27, SEQ ID NO:29, SEQ ID NO:31, SEQ ID NO:33, SEQ ID NO:35, SEQ ID NO:275, SEQ ID NO:277, SEQ ID NO:279, SEQ ID NO:281, SEQ ID NO:321, SEQ ID NO:323, and SEQ ID NO:325.

In some embodiments, the nucleic acid sequence that encodes a bicistronic iCAR/aCAR exhibits at least 99% sequence identity to a nucleic acid sequence selected from the group consisting of SEQ ID NO:1, SEQ ID NO:3, SEQ ID NO:5, SEQ ID NO:7, SEQ ID NO:9, SEQ ID NO:11, SEQ ID NO:13, SEQ ID NO:15, SEQ ID NO:17, SEQ ID NO:19, SEQ ID NO:21, SEQ ID NO:23, SEQ ID NO:25, SEQ ID NO:27, SEQ ID NO:29, SEQ ID NO:31, SEQ ID NO:33, SEQ ID NO:35, SEQ ID NO:275, SEQ ID NO:277, SEQ ID NO:279, SEQ ID NO:281, SEQ ID NO:321, SEQ ID NO:323, and SEQ ID NO:325.

In some embodiments, the nucleic acid sequence that encodes a bicistronic iCAR/aCAR exhibits 100% sequence identity to a nucleic acid sequence selected from the group consisting of SEQ ID NO:1, SEQ ID NO:3, SEQ ID NO:5, SEQ ID NO:7, SEQ ID NO:9, SEQ ID NO:11, SEQ ID NO:13, SEQ ID NO:15, SEQ ID NO:17, SEQ ID NO:19, SEQ ID NO:21, SEQ ID NO:23, SEQ ID NO:25, SEQ ID NO:27, SEQ ID NO:29, SEQ ID NO:31, SEQ ID NO:33, SEQ ID NO:35, SEQ ID NO:275, SEQ ID NO:277, SEQ ID NO:279, SEQ ID NO:281, SEQ ID NO:321, SEQ ID NO:323, and SEQ ID NO:325.

As used herein, sequence identity can include the identity/similarity between two or more nucleic acid sequences, or two or more amino acid sequences, is expressed in terms of the identity or similarity between the sequences. Sequence identity can be measured in terms of percentage identity; the higher the percentage, the more identical the sequences are. Sequence similarity can be measured in terms of percentage similarity (which takes into account conservative amino acid substitutions); the higher the percentage, the more similar the sequences are. Homologs or orthologs of nucleic acid or amino acid sequences possess a relatively high degree of sequence identity/similarity when aligned using standard methods. Methods of alignment of sequences for comparison are well known in the art. Various programs and alignment algorithms are described in, for example but not limited to Smith & Waterman, Adv. Appl. Math. 2:482, 1981; Needleman & Wunsch, J. Mol. Biol. 48:443, 1970; Pearson & Lipman, Proc. Natl. Acad. Sci. USA 85:2444, 1988; Higgins & Sharp, Gene, 73:237-44, 1988; Higgins & Sharp, CABIOS 5:151-3, 1989; Corpet et al., Nuc. Acids Res. 16:10881-90, 1988; Huang et al. Computer Appls. in the Biosciences 8, 155-65, 1992; and Pearson et al., Meth. Mol. Bio. 24:307-31, 1994. Altschul et al., J. Mol. Biol. 215:403-10, 1990, presents a detailed consideration of sequence alignment methods and homology calculations. The NCBI Basic Local Alignment Search Tool (BLAST) (Altschul et al., J. Mol. Biol. 215:403-10, 1990) is available from several sources, including the National Center for Biological Information (NCBI, National Library of Medicine, Building 38A, Room 8N805, Bethesda, Md. 20894) and on the Internet, for use in connection with the sequence analysis programs blastp, blastn, blastx, tblastn and tblastx. Additional information can be found at the NCBI web site. For example, BLASTN can be used to compare nucleic acid sequences, while BLASTP can be used to compare amino acid sequences. To compare two nucleic acid sequences, the options can be set as follows: -i is set to a file containing the first nucleic acid sequence to be compared (such as C:†seq1.txt); --j is set to a file containing the second nucleic acid sequence to be compared (such as C:\seq2.txt); --p is set to blastn; --o is set to any desired file name (such as C:\output.txt); --q is set to --l; --r is set to 2; and all other options are left at their default setting. For example, the following command can be used to generate an output file containing a comparison between two sequences: C:\B12seq --i c:\seq1.txt --j c:\seq2.txt --p blastn --o c:\output.txt --q --1 --r 2.

### IV. Production of Cd4+ or Cd8+ Effector Cells

In still another aspect, the present invention provides a method for preparing a population of CD4+ cells comprising:
(i) obtaining a population of effector immune cells directed to a tumor-associated antigen,
(ii) enriching the effector immune cells for CD4+, and (iii) transfecting the CD4+ effector immune cells with a nucleic acid molecule comprising a nucleotide sequence encoding a bicistronic iCAR/aCAR construct or monocistronic aCAR and iCAR constructs for co-transduction as defined herein above or transducing the cells with a vector; or
(i) obtaining a population of naïve effector immune cells, (ii) enriching the naïve effector immune cells for CD4+, and (iii) transfecting the CD4+ naïve effector immune cell with a nucleic acid molecule comprising a nucleotide sequence encoding a bicistronic iCAR/aCAR construct as defined herein above or transducing the cells with a vector.

In some embodiments, step (ii) is performed before step (iii). In some embodiments, step (iii) is performed before step (ii). In some embodiments, the bicistronic iCAR/aCAR construct is encoded a single vector.

In still another aspect, the present invention provides a method for preparing a population of CD4+ cells comprising:
(i) obtaining a population of TCR-engineered effector immune cells directed to a tumor-associated antigen, (ii) enriching the TCR-engineered effector immune cells for CD4+, and (iii) transfecting the CD4+ TCR-engineered effector immune cells with a nucleic acid molecule comprising a nucleotide sequence encoding a bicistronic iCAR/aCAR construct or monocistronic aCAR and iCAR constructs for co-transduction as defined herein above or transducing the cells with a vector; or
(i) obtaining a population of naïve effector immune cell, (ii) enriching the naïve effector immune cells for CD4+, and (iii) transfecting the CD4+ naïve effector immune cell with a nucleic acid molecule comprising a nucleotide sequence encoding a bicistronic iCAR/aCAR construct as defined herein above or transducing the cells with a vector.

In some embodiments, step (ii) is performed before step (iii). In some embodiments, step (iii) is performed before step (ii). In some embodiments, the bicistronic iCAR/aCAR construct is encoded a single vector. In some embodiments, the bicistronic iCAR and aCAR constructs are encoded on different/separate vectors. In some embodiments, the monocistronic aCAR and iCAR constructs for co-transduction are encoded on a single vector. In some embodiments, the monocistronic aCAR and iCAR constructs for co-transduction are encoded on different/separate vectors.

In still another aspect, the present invention provides a method for preparing a population of CD8+ cells comprising:
(i) obtaining a population of effector immune cells directed to a tumor-associated antigen,
(ii) enriching the effector immune cells for CD8+, and (iii) transfecting the CD8+ effector immune cells with a nucleic acid molecule comprising a nucleotide sequence encoding a bicistronic iCAR/aCAR construct or monocistronic aCAR and iCAR constructs for co-transduction as defined herein above or transducing the cells with a vector; or
(i) obtaining a population of naïve effector immune cells, (ii) enriching the naïve effector immune cells for CD8+, and (iii) transfecting the CD8+ naïve effector immune cell with a nucleic acid molecule comprising a nucleotide sequence encoding a bicistronic iCAR/aCAR construct as defined herein above or transducing the cells with a vector.

In some embodiments, step (ii) is performed before step (iii). In some embodiments, step (iii) is performed before step (ii). In some embodiments, the bicistronic iCAR/aCAR construct is encoded a single vector.

In still another aspect, the present invention provides a method for preparing a population of CD8+ cells comprising:
(i) obtaining a population of TCR-engineered effector immune cells directed to a tumor-associated antigen, (ii) enriching the TCR-engineered effector immune cells for CD8+, and (iii) transfecting the CD8+ TCR-engineered effector immune cells with a nucleic acid molecule comprising a nucleotide sequence encoding a bicistronic iCAR/aCAR construct or monocistronic aCAR and iCAR constructs for co-transduction as defined herein above or transducing the cells with a vector; or
(i) obtaining a population of naïve effector immune cell, (ii) enriching the naïve effector immune cells for CD8+, and (iii) transfecting the CD8+ naïve effector immune cell with a nucleic acid molecule comprising a nucleotide sequence encoding a bicistronic iCAR/aCAR construct as defined herein above or transducing the cells with a vector.

In some embodiments, step (ii) is performed before step (iii). In some embodiments, step (iii) is performed before step (ii). In some embodiments, the bicistronic iCAR/aCAR construct is encoded a single vector. In some embodiments, the bicistronic iCAR and aCAR constructs are encoded on different/separate vectors. In some embodiments, the monocistronic aCAR and iCAR constructs for co-transduction are encoded on a single vector. In some embodiments, the monocistronic aCAR and iCAR constructs for co-transduction are encoded on different/separate vectors.

In some embodiments, the immune cell for use in engineering includes but is not limited to a T-cell, a natural killer cell, or a cytokine-induced killer cell. In some embodiments, the immune cell for use in engineering includes but is not limited to a Jurkat T-cell, a Jurkat-NFAT T-cell, and/or a peripheral blood mononuclear cell (PBMC). In some embodiments, the immune cell for use in engineering is a CD4+ cell. In some embodiments, the immune cell for use in engineering is a CD8+ cell. In some embodiments, the immune cells for use in engineering comprise a combination of CD4+ and CD8+ cells.

In some embodiments, the population of CD4+ cells, CD8+ cells, or a combination thereof, are modified such that they are safe effector CD4+ immune cells, CD8+ immune cells, or a combination thereof. In yet another aspect, the present invention provides a population of CD4+ cells, CD8+ cells, or a combination thereof, obtained by the method of the present invention as described above. The population of CD4+ cells, CD8+ cells, or a combination thereof, may be redirected T cells expressing an exogenous T cell receptor (TCR) and a bicistronic iCAR/aCAR construct or monocistronic aCAR and iCAR constructs for co-transduction, wherein the exogenous TCR is directed to a non-polymorphic cell surface epitope of an antigen or a single allelic variant of a polymorphic cell surface epitope, wherein said epitope is a tumor-associated antigen or is shared at least by cells of related tumor and normal tissue, and a bicistronic iCAR/aCAR construct or monocistronic aCAR and iCAR constructs for co-transduction is as defined above; or the population of CD4+ cells, CD8+ cells, or a combination thereof, may be redirected effector immune cells such as natural killer cells or T cells expressing a bicistronic iCAR/aCAR construct or monocistronic aCAR and iCAR constructs for co-transduction as defined above.

In some embodiments, the population of CD4+ cells, CD8+ cells, or a combination thereof, expresses on their surface an aCAR comprising an extracellular domain that specifically binds to a non-polymorphic cell surface epitope of an antigen and an iCAR comprising an extracellular domain that specifically binds a single allelic variant of a polymorphic cell surface epitope of a different antigen to which the extracellular domain of said aCAR binds. In some embodiments, the extracellular domain of the iCAR specifically binds a single allelic variant of a different polymorphic cell surface epitope are of the same antigen to which the extracellular domain of said aCAR binds; or the extracellular domain of the iCAR specifically binds a different single allelic variant of the same polymorphic cell surface epitope area to which the extracellular domain of said aCAR binds.

In some embodiments, the aCAR and the iCAR are present on the cell surface as separate proteins. In some embodiments, the expression level on the cell surface of the iCAR is greater than or equal to the expression level of the aCAR. In some embodiments, the extracellular domain of the iCAR expressed on the cell surface is directed against or specifically binds to a single allelic variant of an at least one extracellular polymorphic epitope.

In some embodiments, the extracellular domain of the iCAR expressed on the cell surface is directed against or specifically binds to a single allelic variant of HLA-A2. In some embodiments, the iCAR will be directed toward HLA-A2. In some embodiments, the aCAR with be directed toward EGFR. In some embodiments, the aCAR with be directed toward HER2. In some embodiments, the iCAR/aCAR set will be HLA-A2 and EGFR respectively. In some embodiments, the iCAR/aCAR set will be HLA-A2 and HER2 respectively.

In some embodiments, the population of CD4+ cells, CD8+ cells, or a combination thereof, comprises a bicistronic iCAR/aCAR nucleic acid sequence selected from the group consisting of SEQ ID NO:1, SEQ ID NO:3, SEQ ID NO:5, SEQ ID NO:7, SEQ ID NO:9, SEQ ID NO:11, SEQ ID NO:13, SEQ ID NO:15, SEQ ID NO:17, SEQ ID NO:19, SEQ ID NO:21, SEQ ID NO:23, SEQ ID NO:25, SEQ ID NO:27, SEQ ID NO:29, SEQ ID NO:31, SEQ ID NO:33, SEQ ID NO:35, SEQ ID NO:275, SEQ ID NO:277, SEQ ID NO:279, SEQ ID NO:281, SEQ ID NO:321, SEQ ID NO:323, and SEQ ID NO:325.

In some embodiments, the population of CD4+ cells, CD8+ cells, or a combination thereof, comprises and expression vector comprising a bicistronic iCAR/aCAR nucleic acid sequence selected from the group consisting of SEQ ID NO:1, SEQ ID NO:3, SEQ ID NO:5, SEQ ID NO:7, SEQ ID NO:9, SEQ ID NO:11, SEQ ID NO:13, SEQ ID NO:15, SEQ ID NO:17, SEQ ID NO:19, SEQ ID NO:21, SEQ ID NO:23, SEQ ID NO:25, SEQ ID NO:27, SEQ ID NO:29, SEQ ID NO:31, SEQ ID NO:33, SEQ ID NO:35, SEQ ID NO:275, SEQ ID NO:277, SEQ ID NO:279, SEQ ID NO:281, SEQ ID NO:321, SEQ ID NO:323, and SEQ ID NO:325.

In some embodiments, the population of CD4+ cells, CD8+ cells, or a combination thereof, comprises a bicistronic iCAR/aCAR nucleic acid that encodes an amino acid sequence selected from the group consisting of SEQ ID NO:2, SEQ ID NO:4, SEQ ID NO:6, SEQ ID NO:8, SEQ ID NO:10, SEQ ID NO:12, SEQ ID NO:14, SEQ ID NO:16, SEQ ID NO:18, SEQ ID NO:20, SEQ ID NO:22, SEQ ID NO:24, SEQ ID NO:26, SEQ ID NO:28, SEQ ID NO:30, SEQ ID NO:32, SEQ ID NO:34, SEQ ID NO:36, SEQ ID NO:276, SEQ ID NO:278, SEQ ID NO:280, SEQ ID NO:282, SEQ ID NO:322, SEQ ID NO:324, and SEQ ID NO:326.

In some embodiments, the population of CD4+ cells, CD8+ cells, or a combination thereof, comprises expression vector comprises a bicistronic iCAR/aCAR nucleic acid that encodes an amino acid sequence selected from the group consisting of SEQ ID NO:2, SEQ ID NO:4, SEQ ID NO:6, SEQ ID NO:8, SEQ ID NO:10, SEQ ID NO:12, SEQ ID NO:14, SEQ ID NO:16, SEQ ID NO:18, SEQ ID NO:20, SEQ ID NO:22, SEQ ID NO:24, SEQ ID NO:26, SEQ ID NO:28, SEQ ID NO:30, SEQ ID NO:32, SEQ ID NO:34, SEQ ID NO:36, SEQ ID NO:276, SEQ ID NO:278, SEQ ID NO:280, SEQ ID NO:282, SEQ ID NO:322, SEQ ID NO:324, and SEQ ID NO:326.

In some embodiments, EGFR is the aCAR target and HLA is the iCAR target. In some embodiments, HER2 is the aCAR target and HLA is the iCAR target. In some embodiments, the population of CD4+ cells, CD8+ cells, or a combination thereof, used for treating cancer as defined comprises an expression vector. In some embodiments, the iCAR and aCAR are encoded by a bicistronic nucleic acid based expression vector. In some embodiments, the expression vector comprises a nucleic acid sequence a sequence selected from the group consisting of SEQ ID NO:1, SEQ ID NO:3, SEQ ID NO:5, SEQ ID NO:7, SEQ ID NO:9, SEQ ID NO:11, SEQ ID NO:13, SEQ ID NO:15, SEQ ID NO:17, SEQ ID NO:19, SEQ ID NO:21, SEQ ID NO:23, SEQ ID NO:25, SEQ ID NO:27, SEQ ID NO:29, SEQ ID NO:31, SEQ ID NO:33, SEQ ID NO:3, SEQ ID NO:275, SEQ ID NO:277, SEQ ID NO:279, SEQ ID NO:281, SEQ ID NO:321, SEQ ID NO:323, and SEQ ID NO:325. In some embodiments, the expression vector comprises a nucleic acid sequence that codes for an amino sequence selected from the group consisting of SEQ ID NO:2, SEQ ID NO:4, SEQ ID NO:6, SEQ ID NO:8, SEQ ID NO:10, SEQ ID NO:12, SEQ ID NO:14, SEQ ID NO:16, SEQ ID NO:18, SEQ ID NO:20, SEQ ID NO:22, SEQ ID NO:24, SEQ ID NO:26, SEQ ID NO:28, SEQ ID NO:30, SEQ ID NO:32, SEQ ID NO:34, SEQ ID NO:36, SEQ ID NO:276, SEQ ID NO:278, SEQ ID NO:280, SEQ ID NO:282, SEQ ID NO:322, SEQ ID NO:324, and SEQ ID NO:326.

In some embodiments, the population of CD4+ cells, CD8+ cells, or a combination thereof, used for treating cancer comprises an expression vector that comprises a bicistronic iCAR/aCAR nucleic acid sequence selected from the group consisting of SEQ ID NO:1, SEQ ID NO:3, SEQ ID NO:5, SEQ ID NO:7, SEQ ID NO:9, SEQ ID NO:11, SEQ ID NO:13, SEQ ID NO:15, SEQ ID NO:17, SEQ ID NO:19, SEQ ID NO:21, SEQ ID NO:23, SEQ ID NO:25, SEQ ID NO:27, SEQ ID NO:29, SEQ ID NO:31, SEQ ID NO:33, SEQ ID NO:35, SEQ ID NO:275, SEQ ID NO:277, SEQ ID NO:279, SEQ ID NO:281, SEQ ID NO:321, SEQ ID NO:323, and SEQ ID NO:325.

In some embodiments, the population of CD4+ cells, CD8+ cells, or a combination thereof, used for treating cancer comprises a bicistronic iCAR/aCAR nucleic acid sequence selected from the group consisting of SEQ ID NO:1, SEQ ID NO:3, SEQ ID NO:5, SEQ ID NO:7, SEQ ID NO:9, SEQ ID NO:11, SEQ ID NO:13, SEQ ID NO:15, SEQ ID NO:17, SEQ ID NO:19, SEQ ID NO:21, SEQ ID NO:23, SEQ ID NO:25, SEQ ID NO:27, SEQ ID NO:29, SEQ ID NO:31, SEQ ID NO:33, SEQ ID NO:35, SEQ ID NO:275, SEQ ID NO:277, SEQ ID NO:279, SEQ ID NO:281, SEQ ID NO:321, SEQ ID NO:323, and SEQ ID NO:325.

In some embodiments, the population of CD4+ cells, CD8+ cells, or a combination thereof, used for treating cancer as comprises a bicistronic iCAR/aCAR nucleic acid that encodes an amino acid sequence selected from the group consisting of SEQ ID NO:2, SEQ ID NO:4, SEQ ID NO:6, SEQ ID NO:8, SEQ ID NO:10, SEQ ID NO:12, SEQ ID NO:14, SEQ ID NO:16, SEQ ID NO:18, SEQ ID NO:20, SEQ ID NO:22, SEQ ID NO:24, SEQ ID NO:26, SEQ ID NO:28, SEQ ID NO:30, SEQ ID NO:32, SEQ ID NO:34, SEQ ID NO:36, SEQ ID NO:276, SEQ ID NO:278, SEQ ID NO:280, SEQ ID NO:282, SEQ ID NO:322, SEQ ID NO:324, and SEQ ID NO:326.

In some embodiments, the population of CD4+ cells, CD8+ cells, or a combination thereof, used for treating cancer as comprises an expression vector that comprises a bicistronic iCAR/aCAR nucleic acid that encodes an amino acid sequence selected from the group consisting of SEQ ID NO:2, SEQ ID NO:4, SEQ ID NO:6, SEQ ID NO:8, SEQ ID NO:10, SEQ ID NO:12, SEQ ID NO:14, SEQ ID NO:16, SEQ ID NO:18, SEQ ID NO:20, SEQ ID NO:22, SEQ ID NO:24, SEQ ID NO:26, SEQ ID NO:28, SEQ ID NO:30, SEQ ID NO:32, SEQ ID NO:34, SEQ ID NO:36, SEQ ID NO:276, SEQ ID NO:278, SEQ ID NO:280, SEQ ID NO:282, SEQ ID NO:322, SEQ ID NO:324, and SEQ ID NO:326.

In some embodiments, a population of CD4+ cells is produced. In some embodiments, a population of CD4+ cells is administered to a patient. In some embodiments, a population of CD8+ cells is produced. In some embodiments, a population of CD8+ cells is administered to a patient. In some embodiments, a population of a combination of CD4+ cells and CD8+ cells is produced. In some embodiments, a population of a combination of CD4+ cells and CD8+ cells is administered to a patient.

In some embodiments, a combination of CD4+ cells and CD8+ cells comprises about 100% CD4+ cells and about 0% CD8+ cells. In some embodiments, a combination of CD4+ cells and CD8+ cells comprises about 95% CD4+ cells and about 5% CD8+ cells. In some embodiments, a combination of CD4+ cells and CD8+ cells comprises about 90% CD4+ cells and about 10% CD8+ cells. In some embodiments, a combination of CD4+ cells and CD8+ cells comprises about 85% CD4+ cells and about 15% CD8+ cells. In some embodiments, a combination of CD4+ cells and CD8+ cells comprises about 80% CD4+ cells and about 20% CD8+ cells. In some embodiments, a combination of CD4+ cells and CD8+ cells comprises about 75% CD4+ cells and about 25% CD8+ cells. In some embodiments, a combination of CD4+ cells and CD8+ cells comprises about 70% CD4+ cells and about 30% CD8+ cells. In some embodiments, a combination of CD4+ cells and CD8+ cells comprises about 65% CD4+ cells and about 35% CD8+ cells. In some embodiments, a combination of CD4+ cells and CD8+ cells comprises about 60% CD4+ cells and about 40% CD8+ cells. In some embodiments, a combination of CD4+ cells and CD8+ cells comprises about 55% CD4+ cells and about 45% CD8+ cells. In some embodiments, a combination of CD4+ cells and CD8+ cells comprises about 50% CD4+ cells and about 50% CD8+ cells. In some embodiments, a combination of CD4+ cells and CD8+ cells comprises about 45% CD4+ cells and about 55% CD8+ cells. In some embodiments, a combination of CD4+ cells and CD8+ cells comprises about 40% CD4+ cells and about 60% CD8+ cells. In some embodiments, a combination of CD4+ cells and CD8+ cells comprises about 35% CD4+ cells and about 65% CD8+ cells. In some embodiments, a combination of CD4+ cells and CD8+ cells comprises about 30% CD4+ cells and about 70% CD8+ cells. In some embodiments, a combination of CD4+ cells and CD8+ cells comprises about 25% CD4+ cells and about 75% CD8+ cells. In some embodiments, a combination of CD4+ cells and CD8+ cells comprises about 20% CD4+ cells and about 80% CD8+ cells. In some embodiments, a combination of CD4+ cells and CD8+ cells comprises about 15% CD4+ cells and about 85% CD8+ cells. In some embodiments, a combination of CD4+ cells and CD8+ cells comprises about 10% CD4+ cells and about 90% CD8+ cells. In some embodiments, a combination of CD4+ cells and CD8+ cells comprises about 5% CD4+ cells and about 95% CD8+ cells. In some embodiments, a combination of CD4+ cells and CD8+ cells comprises about 0% CD4+ cells and about 100% CD8+ cells.

In some embodiments, a combination of CD4+ cells and CD8+ cells comprises a ratio of CD4+ cells to CD8+ cells of about 20:1 to about 1:20. In some embodiments, a combination of CD4+ cells and CD8+ cells comprises a ratio of CD4+ cells to CD8+ cells of about 15:1 to about 1:15. In some embodiments, a combination of CD4+ cells and CD8+ cells comprises a ratio of CD4+ cells to CD8+ cells of about 10:1 to about 1:10. In some embodiments, a combination of CD4+ cells and CD8+ cells comprises a ratio of CD4+ cells to CD8+ cells of about 9:1 to about 1:9. In some embodiments, a combination of CD4+ cells and CD8+ cells comprises a ratio of CD4+ cells to CD8+ cells of about 8:1 to about 1:8. In some embodiments, a combination of CD4+ cells and CD8+ cells comprises a ratio of CD4+ cells to CD8+ cells of about 7:1 to about 1:7. In some embodiments, a combination of CD4+ cells and CD8+ cells comprises a ratio of CD4+ cells to CD8+ cells of about 6:1 to about 1:6. In some embodiments, a combination of CD4+ cells and CD8+ cells comprises a ratio of CD4+ cells to CD8+ cells of about 5:1 to about 1:5. In some embodiments, a combination of CD4+ cells and CD8+ cells comprises a ratio of CD4+ cells to CD8+ cells of about 4:1 to about 1:4. In some embodiments, a combination of CD4+ cells and CD8+ cells comprises a ratio of CD4+ cells to CD8+ cells of about 3:1 to about 1:3. In some embodiments, a combination of CD4+ cells and CD8+ cells comprises a ratio of CD4+ cells to CD8+ cells of about 2:1 to about 1:2. In some embodiments, a combination of CD4+ cells and CD8+ cells comprises a ratio of CD4+ cells to CD8+ cells of about 1:1.

In some embodiments, a combination of CD4+ cells and CD8+ cells comprises a ratio of CD4+ cells to CD8+ cells of about 20:1. In some embodiments, a combination of CD4+ cells and CD8+ cells comprises a ratio of CD4+ cells to CD8+ cells of about 19:1. In some embodiments, a combination of CD4+ cells and CD8+ cells comprises a ratio of CD4+ cells to CD8+ cells of about 18:1. In some embodiments, a combination of CD4+ cells and CD8+ cells comprises a ratio of CD4+ cells to CD8+ cells of about 17:1. In some embodiments, a combination of CD4+ cells and CD8+ cells comprises a ratio of CD4+ cells to CD8+ cells of about 16:1. In some embodiments, a combination of CD4+ cells and CD8+ cells comprises a ratio of CD4+ cells to CD8+ cells of about 15:1. In some embodiments, a combination of CD4+ cells and CD8+ cells comprises a ratio of CD4+ cells to CD8+ cells of about 14:1. In some embodiments, a combination of CD4+ cells and CD8+ cells comprises a ratio of CD4+ cells to CD8+ cells of about 13:1. In some embodiments, a combination of CD4+ cells and CD8+ cells comprises a ratio of CD4+ cells to CD8+ cells of about 12:1. In some embodiments, a combination of CD4+ cells and CD8+ cells comprises a ratio of CD4+ cells to CD8+ cells of about 11:1. In some embodiments, a combination of CD4+ cells and CD8+ cells comprises a ratio of CD4+ cells to CD8+ cells of about 10:1. In some embodiments, a combination of CD4+ cells and CD8+ cells comprises a ratio of CD4+ cells to CD8+ cells of about 9:1. In some embodiments, a combination of CD4+ cells and CD8+ cells comprises a ratio of CD4+ cells to CD8+ cells of about 8:1. In some embodiments, a combination of CD4+ cells and CD8+ cells comprises a ratio of CD4+ cells to CD8+ cells of about 7:1. In some embodiments, a combination of CD4+ cells and CD8+ cells comprises a ratio of CD4+ cells to CD8+ cells of about 6:1. In some embodiments, a combination of CD4+ cells and CD8+ cells comprises a ratio of CD4+ cells to CD8+ cells of about 5:1. In some embodiments, a combination of CD4+ cells and CD8+ cells comprises a ratio of CD4+ cells to CD8+ cells of about 4:1. In some embodiments, a combination of CD4+ cells and CD8+ cells comprises a ratio of CD4+ cells to CD8+ cells of about 2:1. In some embodiments, a combination of CD4+ cells and CD8+ cells comprises a ratio of CD4+ cells to CD8+ cells of about 2:1. In some embodiments, a combination of CD4+ cells and CD8+ cells comprises a ratio of CD4+ cells to CD8+ cells of about 1:1.

In some embodiments, a combination of CD4+ cells and CD8+ cells comprises a ratio of CD4+ cells to CD8+ cells of about 1:2. In some embodiments, a combination of CD4+ cells and CD8+ cells comprises a ratio of CD4+ cells to CD8+ cells of about 1:3. In some embodiments, a combination of CD4+ cells and CD8+ cells comprises a ratio of CD4+ cells to CD8+ cells of about 1:4. In some embodiments, a combination of CD4+ cells and CD8+ cells comprises a ratio of CD4+ cells to CD8+ cells of about 1:5. In some embodiments, a combination of CD4+ cells and CD8+ cells comprises a ratio of CD4+ cells to CD8+ cells of about 1:6. In some embodiments, a combination of CD4+ cells and CD8+ cells comprises a ratio of CD4+ cells to CD8+ cells of about 1:7. In some embodiments, a combination of CD4+ cells and CD8+ cells comprises a ratio of CD4+ cells to CD8+ cells of about 1:8. In some embodiments, a combination of CD4+ cells and CD8+ cells comprises a ratio of CD4+ cells to CD8+ cells of about 1:9. In some embodiments, a combination of CD4+ cells and CD8+ cells comprises a ratio of CD4+ cells to CD8+ cells of about 1:10. In some embodiments, a combination of CD4+ cells and CD8+ cells comprises a ratio of CD4+ cells to CD8+ cells of about 1:11. In some embodiments, a combination of CD4+ cells and CD8+ cells comprises a ratio of CD4+ cells to CD8+ cells of about 1:12. In some embodiments, a combination of CD4+ cells and CD8+ cells comprises a ratio of CD4+ cells to CD8+ cells of about 1:13. In some embodiments, a combination of CD4+ cells and CD8+ cells comprises a ratio of CD4+ cells to CD8+ cells of about 1:14. In some embodiments, a combination of CD4+ cells and CD8+ cells comprises a ratio of CD4+ cells to CD8+ cells of about 1:15. In some embodiments, a combination of CD4+ cells and CD8+ cells comprises a ratio of CD4+ cells to CD8+ cells of about 1:16. In some embodiments, a combination of CD4+ cells and CD8+ cells comprises a ratio of CD4+ cells to CD8+ cells of about 1:17. In some embodiments, a combination of CD4+ cells and CD8+ cells comprises a ratio of CD4+ cells to CD8+ cells of about 1:18. In some embodiments, a combination of CD4+ cells and CD8+ cells comprises a ratio of CD4+ cells to CD8+ cells of about 1:19. In some embodiments, a combination of CD4+ cells and CD8+ cells comprises a ratio of CD4+ cells to CD8+ cells of about 1:20.

### A. In Vitro Assays

In some embodiments, the bicistronic iCAR/aCAR constructs will be tested for activity effects, including effectiveness and ability to inhibit, using a variety of assays. In some embodiments, the bicistronic iCAR/aCAR constructs or monocistronic aCAR and iCAR constructs for co-transduction will be tested in-vitro and/or in-vivo. In some embodiments, the bicistronic iCAR/aCAR constructs or monocistronic aCAR and iCAR constructs for co-transduction will be tested in-vitro. In some embodiments, the bicistronic iCAR/aCAR constructs or monocistronic aCAR and iCAR constructs for co-transduction will be tested in-vivo. In some embodiments, the in vitro assays measure cytokine secretion and/or cytotoxicity effects. In some embodiments, the in vivo assays will evaluate the bicistronic iCAR/aCAR constructs or monocistronic aCAR and iCAR constructs for co-transduction inhibition and protection to on-target off tumor xenografts. In some embodiments, the in vivo assays will evaluate the bicistronic iCAR/aCAR constructs or monocistronic aCAR and iCAR constructs for co-transduction inhibition and protection to on-target off tumor tissue and/or viral organs.

i. Luciferase Cytotoxicity Assay

In some embodiments, bicistronic iCAR/aCAR constructs or monocistronic aCAR and iCAR constructs for co-transduction are evaluated using a luciferase cytotoxicity assay. Generally, for a luciferase cytotoxic assay, target tumor cells (which can be referred to as “T”) are engineered to express firefly luciferase. In some embodiments, commercially available ATCC cell lines are used. In some embodiments, H1703 cells were used. In some embodiments, H1650 cells were used. In some embodiments, H1792 cells were used. In some embodiments, H292 cells were used. The in vitro luciferase assay can be performed according to the Bright-Glo Luciferase assay (commercially available from Promega or BPS Biosciences or other commercial vendors). Transduced effector (E) T cells (which have been transduced with bicistronic iCAR/aCAR constructs or mock/control construct) can be incubated for 18-48 hrs with recombinant target cells expressing the iCAR or aCAR target to be tested in different effector to target ratios. In some embodiments, the iCAR/aCAR pair comprises any of aCAR and/or iCAR with the components as described above. In some embodiments, the bicistronic iCAR/aCAR constructs described above are to be tested. In some embodiments, the bicistronic iCAR/aCAR comprises an amino acid sequence encoded by a nucleic acid sequence selected from the group consisting of SEQ ID NO:1, SEQ ID NO:3, SEQ ID NO:5, SEQ ID NO:7, SEQ ID NO:9, SEQ ID NO:11, SEQ ID NO:13, SEQ ID NO:15, SEQ ID NO:17, SEQ ID NO:19, SEQ ID NO:21, SEQ ID NO:23, SEQ ID NO:25, SEQ ID NO:27, SEQ ID NO:29, SEQ ID NO:31, SEQ ID NO:33, SEQ ID NO:35, SEQ ID NO:275, SEQ ID NO:277, SEQ ID NO:279, SEQ ID NO:281, SEQ ID NO:321, SEQ ID NO:323, and SEQ ID NO:325. In some embodiments, the bicistronic iCAR/aCAR comprises an amino acid sequence selected from the group consisting of SEQ ID NO:2, SEQ ID NO:4, SEQ ID NO:6, SEQ ID NO:8, SEQ ID NO:10, SEQ ID NO:12, SEQ ID NO:14, SEQ ID NO:16, SEQ ID NO:18, SEQ ID NO:20, SEQ ID NO:22, SEQ ID NO:24, SEQ ID NO:26, SEQ ID NO:28, SEQ ID NO:30, SEQ ID NO:32, SEQ ID NO:34, SEQ ID NO:36, SEQ ID NO:276, SEQ ID NO:278, SEQ ID NO:280, SEQ ID NO:282, SEQ ID NO:322, SEQ ID NO:324, and SEQ ID NO:326. Cell killing can be quantified indirectly by estimating the number of live cells with the Bright-Glo Luciferase system. Cell killing can also be measured using an IncuCyte cytotoxicity assay.

In some embodiments, the ‘off-tumor’ cytotoxicity can be manipulated by sorting transduced T cell populations according to iCAR/aCAR expression level or by selecting a sub population of recombinant target cells according to their target expression, including for example, expression of the gene product encoding for at least one extracellular polymorphic epitope. In some embodiments, the aCAR and iCAR target is any target with an extracellular domain. In some embodiments, the sorting is based on EGFR, HER2, or HLA-A2 expression level.

In some embodiments, the bicistronic iCAR/aCAR construct or monocistronic aCAR and iCAR constructs for co-transduction is examined to determine whether the iCAR transduced T cells can discriminate between the ‘on-tumor’ cells (e.g., tumor cells) and ‘off-tumor’ cells (e.g., non-tumor cells) in vitro. Generally, this is tested by examining the killing effect of transduced T cells incubated with a mix of ‘on-tumor’ and ‘off-tumor’ cells at a ratio of 1:1 to 1:10. In some embodiments, the ratio Target cells to Effector T cells (T:E ratio) is 1:0.02, 1:0.04, 1:0.06, 1:0.08, 1:0.1, 1:0.12, 1:0.12, 1:0.14, 1:0.16, 1:0.18, 1:2, 1:3, 1:4, 1:5, 1:6, 1:7, 1:8, 1:9, 1:10, 1:11, 1:12, 1:13, 1:14, 1:15, 1:16, 1:17, 1:18, 1:19, or 1:20. In some embodiments, the E:T ratio (Effector T cells to Target cells) is 0.02:1, 0.04:1, 0.06:1, 0.08:1, 0.1:1, 0.12:1, 0.12:1, 0.14:1, 0.16:1, 0.18:1, 2:1, 3:1, 4:1, 5:1:1, 6:1, 7:1, 8:1, 9:1, 10:1, 11:1, 12:1, 13:1, 14:1, 15:1, 16:1, 17:1, 18:1, 19:1, or 20:1. The on tumor recombinant cells can be distinguished from the ‘off-tumor’ recombinant cells by luciferase expression in embodiments where only one cell population will be engineered to express the luciferase gene at a time). Killing can be quantified after 24-48 hrs of co-incubation using the Bright-Glo Luciferase assay (Promega). Killing can also be quantified using an IncCyte cytotoxicity assay. In some embodiments, transduced cells were only used in the assay of transduction efficiency was greater than 10% and expression was observed for both aCAR and iCAR.

In some embodiments, the bicistronic iCAR/aCAR construct or monocistronic aCAR and iCAR constructs for co-transduction transduced T cells exhibit about 10%, about 20%, about 30%, about 40%, about 50%, about 60%, about 70%, about 80%, about 90%, and/or about 95% less off-tumor cell killing as compared to T cells transduced with aCAR (or other control) but not transduced with the bicistronic iCAR/aCAR construct or monocistronic aCAR and iCAR constructs for co-transduction. In some embodiments, the bicistronic iCAR/aCAR construct or monocistronic aCAR and iCAR constructs for co-transduction transduced T cells exhibit about 1-fold, about 2-fold, about 3-fold, about 4-fold, about 5-fold, or about 10-fold less off-tumor cell killing as compared to T cells transduced with aCAR (or other control) but not transduced with the bicistronic iCAR/aCAR construct.

ii. Caspase 3

In some embodiments, caspase 3-detection assays are employed to determine the level of apoptosis of the ‘on-tumor’ cells (e.g., tumor cells) and ‘off-tumor’ cells (e.g., non-tumor cells) in vitro. In some embodiments, caspase_3-detection of cytotoxic lymphocyte (CTL) induced apoptosis by an antibody to activated cleaved caspase 3 is examined.

Generally, one of the pathways by which CTLs kill target cells is by inducing apoptosis through the Fas ligand. The CASP3 protein is a member of the cysteine-aspartic acid protease (caspase) family. Typically, sequential activation of caspases plays a significant role in the execution-phase of cell apoptosis and as such, cleavage of pro-caspase 3 to caspase 3 results in conformational change and expression of catalytic activity. The cleaved activated form of caspase 3 can be recognized specifically by a monoclonal antibody.

In some embodiments, transduced T cells can be incubated with either ‘on-tumor’ (e.g., mimicking tumor) and ‘off-tumor’ cells (e.g., mimicking non-tumor) recombinant cells. In some embodiments, the ‘on-tumor’ (e.g., tumor) and ‘off-tumor’ cells (e.g., non-tumor) recombinant cells have been previously labeled with CFSE ((5(6)-Carboxyfluorescein N-hydroxysuccinimidyl ester)) or other cell tracer dye (e.g., CellTrace Violet). In some embodiments, co-incubation of target cells with effector cells occurs for about 1 hour to 6 about hours, about 2 hours to about 5 hours, or about 2 to about 4 hrs. In some embodiments, target cell apoptosis is quantified by flow cytometry. Cells can be permeabilized and fixed by an inside staining kit (Miltenyi or BD bioscience) and stained with an antibody for activated caspase 3 (BD bioscience).

In some embodiments, the bicistronic iCAR/aCAR construct transduced T cells induce about 10%, about 20%, about 30%, about 40%, about 50%, about 60%, about 70%, about 80%, about 90%, and/or about 95% less off-tumor cell apoptosis as compared to T cells transduced with the bicistronic iCAR/aCAR construct but not transduced with the iCAR (or other appropriate control). In some embodiments, the bicistronic iCAR/aCAR construct transduced T cells induce about 1-fold, about 2-fold, about 3-fold, about 4-fold, about 5-fold, or about 10-fold less off-tumor cell apoptosis as compared to T cells transduced with aCAR (or other control) but not transduced with the bicistronic iCAR/aCAR construct.

iii. Time-Lapse Microscopy

Time lapse microscopy of the bicistronic iCAR/aCAR construct or monocistronic aCAR and iCAR constructs for co-transduction transduced T cells can be employed in order to discern target binding. In some embodiments, target cells will be labeled with a reporter gene (for example but not limited to a fluorescent protein such as nucGFP). In some embodiments, transduced T cells are incubated with either ‘on-tumor’ or ‘off-tumor’ cells for up to 5 days. In some embodiments, time lapse microscopy can be used to visualize killing. In some embodiments, flow cytometry analysis using viable cell number staining and CountBright™ beads (commercially available from Thermofisher/Invitrogen) for determining target cell number at end-point time will be conducted.

In some embodiments, in order to determine if the bicistronic iCAR/aCAR construct or monocistronic aCAR and iCAR constructs for co-transduction transduced T cells can discern targets in vitro, each recombinant target cells (‘on-tumor’ or ‘off-tumor’) is labeled with a different reporter protein (for example GFP and mCherry). In some embodiments, any report protein pair would work, so long as the reporter pair contains two reporters which are easily distinguishable. In some embodiments, transduced T cells (Effector cells) will be co-incubated with the recombinant cells (target cells) at a 1:1 ratio of E/T. In some embodiments, the ration of effector to target (E/T) includes but is not limited to 16:1, 15:1, 14:1, 13:1, 12:1, 11:1, 10:1, 9:1, 8:1, 6:1, 4:1, 2:1, or 1:1. In some embodiments, the cell fate is then examined by microscopy imaging.

iv. Cytokine Expression Intra Cellular Staining

Cytokine expression and/or release can be examined in order to determine T cells activation. In some embodiments, a bicistronic iCAR/aCAR construct transduced T cells are incubated with the recombinant target cells and cytokine production for one or more cytokines is quantified, for example, either by measuring cytokine secretion in cell culture supernatant according to or by flow cytometry analysis, or by Luminex and/or MSD. For the flow cytometry analysis, a Golgi stop can be employed to prevent the secretion of the cytokines. In some embodiments, following a 6 hour and 18 hour to 24 hour incubation of the transduced T cells with target cells, T cells will be permeabilized and fixed by an intracellular staining kit (Miltenyi) and stained with antibodies for the T cell markers (CD3 and CD8) and for one or more cytokines. In some embodiments, the cytokines include but are not limited to IL-2, INFγ, and/or TNFα. In some embodiments, the cytokines are secreted and include but are not limited to IL-2, INFγ, and/or TNFα. In some embodiments, the cytokines are intracellular and include but are not limited to IL-2, INFγ, and/or TNFα.

v. T Cell Degranulation Assay Measured by CD107a Staining

Staining for CD107a can also be examined as a surrogate for cytolytic activity of the transduced T cells. Generally, degranulating of T cells can be identified by the surface expression of CD107a, a lysosomal associated membrane protein (LAMP-1), and surface expression of LAMP-1 has been shown to correlate with CD8 T cell cytotoxicity. Further, this molecule is located on the luminal side of lysosomes. Typically, upon activation, CD107a is transferred to the cell membrane surface of activated lymphocytes. Moreover, CD107a is expressed on the cell surface transiently and is rapidly re-internalized via the endocytic pathway. Therefore, while not being bound by theory, CD107a detection is maximized by antibody staining during cell stimulation and by the addition of monensin (for example, to prevent acidification and subsequent degradation of endocytosed CD107a antibody complexes).

In some embodiments, the bicistronic iCAR/aCAR construct transduced T cells are incubated with the target cells for about 6 hours to about 24 hours and CD107a expression on the CD8 T cells is examined. In some embodiments, the target cells expresso only one target protein recognized by aCAR (as in tumor cells) or target cells expressing both target proteins recognized by aCAR and iCAR (as in normal cells). In some embodiments, the bicistronic iCAR/aCAR construct transduced T cells are incubated with the target cells for about 6 ours to about 24 hrs in the presence of monensin and CD107a expression on the CD8 T cells is followed by flow cytometry using conjugated antibodies against the T cell surface markers (for example, CD3 and CD8) and a conjugated antibody for CD107a.

vi. Quantitation of Secreted Cytokines by ELISA/Luminex

In some embodiments, following co-cultivation of bicistronic iCAR/aCAR construct transduced T-cells (Jurkat, or primary T-cells) expressing iCAR or aCAR or both aCAR and iCAR with modified target cells, expressing iCAR or aCAR or both aCAR and iCAR antigens on their cell surface, conditioned medium will be collected, and cytokine's concentration will be measured by cytokine ELISA or by Luminex xMAP Multiplex Assay technology (Luminex). In some embodiments, the cytokine is selected from the group consisting of IL-2, INFγ and/or TNF□. In some embodiments, the cytokine is selected from the group consisting of IL-2. In some embodiments, the cytokine is selected from the group consisting of INFγ. In some embodiments, the cytokine is selected from the group consisting of TNF□. In some embodiments, a decrease of about 20%, about 25%, about 30%, about 35%, about 40%, about 45%, about 50%, about 55%, about 60%, about 65%, about 70%, about 75%, about 80%, about 85%, about 90%, about 95%, or about 99% is demonstrated with bicistronic iCAR/aCAR construct transduced cells.

vii. Cytokines Secretion Measured by Cytometric Bead Array (CBA) Assay

Cytometric Bead Array (CBA) is used to measure a variety of soluble and intracellular proteins, including cytokines, chemokines and growth factors. In some embodiments, T-cells (primary T-cells or Jurkat cells) transduced with aCAR or both aCAR and iCAR constructs (Effector cells) are stimulated with modified target cells expressing both iCAR and aCAR or aCAR or iCAR target antigens on their cell surface. In some embodiments, the effector to target ratio ranges from 20:1 up to 1:1. In some embodiments, the effector to target ratio ranges from 20:1, 19:1, 18:1, 17:1, 16:1, 15:1, 14:1, 13:1, 12:1, 11:1, 10:1, 9:1, 8:1, 7:1, 6:1, 5:1, 4:1, 3:1, 2:1, or 1:1. In some embodiments, following several hours of co-incubation the effector cells produce and secrete cytokines which indicate their effector state. In some embodiments, the supernatant of the reaction is collected, and secreted IL-2, IFN-γ, and/or TNFα were measured and quantified by multiplex CBA assay.

In some embodiments, a decrease of about 20%, about 25%, about 30%, about 35%, about 40%, about 45%, about 50%, about 55%, about 60%, about 65%, about 70%, about 75%, about 80%, about 85%, about 90%, about 95%, or about 99% is demonstrated with dual CAR (aCAR/iCAR) transduced cells were co-incubated with target cells expressing both target antigens as compared to IL-2, IFN-γ, and/or TNFα secretion resulted from co-incubation of the same effector cells with target cells expressing only one target. In some embodiments, a decrease of about 20%, about 25%, about 30%, about 35%, about 40%, about 45%, about 50%, about 55%, about 60%, about 65%, about 70%, about 75%, about 80%, about 85%, about 90%, about 95%, or about 99% in IL-2 IFN-γ, and/or TNFα secretion was demonstrated when bicistronic iCAR/aCAR construct transduced cells were co-incubated with target cells expressing both target antigens as compared to IL-2 IFN-γ, and/or TNFα secretion resulted from co-incubation of the same effector cells with target cells expressing only one target. In some embodiments, a decrease of 86%.

### B. In Vivo Assays

In some embodiments, the bicistronic iCAR/aCAR construct are tested for effectiveness in vivo. In some embodiments, NOD/SCID/7c- or similar mice are inoculated subcutaneously or orthotopically with tumor cells. In some embodiments, the tumor cells are EGFR and HER2 positive cells lines A549, A431, Fadu, SK-OV-3, U-87, MCF7, NCI-H460, NCI-H1703, NCI-H1650, NCI-H1975, NCI-H292 (ATCC cell lines) cells. In some embodiments, for establishment of and/or differentiation between ‘on-target’ cells and ‘off-tumor’ cells, A549, A431, Fadu, SK-OV-3, U-87, MCF7, NCI-H460 NCI-H1703, NCI-H1650, NCI-H1975, NCI-H292 can be engineered to be deficient or express the iCAR epitope, thereby representing the healthy cells. In some embodiments, the iCAR epitope comprises at least one extracellular polymorphic epitope. In some embodiments, the iCAR epitope is from HLA (including, for example, HLA-A2, HLA-A3, HLA-A, HLA-B, HLA-C, HLA-G, HLA-E, HLA-F, HLA-DPA1, HLA-DQA1, HLA-DQB1, HLA-DQB2, HLA-DRB1, or HLA-DRB5). In some embodiments, the iCAR epitope is from HLA-A2. Other cells that could be employed in these assays include but are not limited to Raji or any other recombinant cell lines. In some embodiments, such assays can be in a PDX (patient derived xenograft) model.

For the assay, mice will be divided into study groups; one cohort will be injected with the A549, A431, Fadu, SK-OV-3, U-87, MCF7, NCI-H460 NCI-H1703, NCI-H1650, NCI-H1975, and/or NCI-H292 cells not expressing the iCAR epitope, while the other will be injected with the corresponding A549, A431, Fadu, SK-OV-3, U-87, MCF7, NCI-H460 NCI-H1703, NCI-H1650, NCI-H1975, NCI-H292 cells expressing the iCAR epitope. Following staging, mice will be infused intravenously with T cells (Untouched, CD4+ only, CD8+ only, or an admix of CD4+ and CD8+) transduced with aCAR, aCAR/iCAR and a control group of untransduced T cells or no T cells. Tumor burden will be measured by through measurement of the subcutaneous tumor volume.

According to one embodiment of the assay, in order to test whether the T cells expressing the bicistronic iCAR/aCAR constructs could discriminate between the target cells and off target cells in vivo within the same organism, mice are injected with a 1:1 mixture of the ‘on-tumor’/’off-tumor A549, A431, Fadu, SK-OV-3, U-87, MCF7, NCI-H460 NCI-H1703, NCI-H1650, NCI-H1975, and/or NCI-H292 cells, followed by injection of transduced T cells expressing either the aCAR alone or both aCAR and iCAR (including as the bicistronic iCAR/aCAR constructs as described herein) after staging. With this embodiment, upon sacrifice of the mice the presence of the ‘on-tumor’ and ‘off-tumor cells Will be evaluated by immunohistochemical staining

According to one embodiment of the assay, in order to test whether the T cells expressing the bicistronic iCAR/aCAR constructs could discriminate between the target cells and off target cells in vivo within the same organism, mice are injected with a 1:10 mixture of the ‘on-tumor’/‘off-tumor NALM-6, A549, A431, Fadu, SK-OV-3, U-87, MCF7, and/or NCI-H460 NCI-H1703, NCI-H1650, NCI-H1975, NCI-H292 cells, followed by injection of transduced T cells expressing either the aCAR alone or both aCAR and iCAR. With this embodiment, upon sacrifice of the mice the presence of the ‘on-tumor’ and ‘off-tumor cells in the spleen and bone marrow will be analyzed by flow cytometry for iCAR and aCAR markers.

i. Tumor Growth Kinetics in Human Xenograft Mouse Models

In some embodiments, the tumor cells express either the iCAR target, aCAR target or both. In some embodiments, an aCAR tumor cell line could be the EGFR or HER2 positive cells lines A549, A431, Fadu, SK-OV-3 U-87, MCF7, and/or NCI-H460 (ATCC cell lines). In some embodiments, tumor cells that express both the aCAR and iCAR (i.e. ‘off-tumor’ cells) are NALM 6, A549, A431, Fadu, SK-OV-3, U-87, MCF7, MDA-MB-231, and/or NCI-H460 engineered to express the iCAR epitope (for example, HLA-A2) thereby representing the healthy cells. In some embodiments, NALM 6 and NALM 6-HLA-A2 can also be engineered to express a reporter gene (e.g., firefly luciferase, GFP, mCherry), for easy detection. In some embodiments, A549 and A549-HLA-A2 can also be engineered to express a reporter gene (e.g., firefly luciferase), for easy detection. In some embodiments, A431 and A431-HLA-A2 can also be engineered to express a reporter gene (e.g., firefly luciferase), for easy detection. In some embodiments, Fadu and Fadu-HLA-A2 can also be engineered to express a reporter gene (e.g., firefly luciferase), for easy detection. In some embodiments, SK-OV-3 and SK-OV-3-HLA-A2 can also be engineered to express a reporter gene (e.g., firefly luciferase), for easy detection. In some embodiments, NCI-H460 and NCI-H460-HLA-A2 can also be engineered to express a reporter gene (e.g., firefly luciferase), for easy detection. In some embodiments, U-87 and U-87-HLA-A2 can also be engineered to express a reporter gene (e.g., firefly luciferase), for easy detection. In some embodiments, MCF7 and MCF7-HLA-A2 can also be engineered to express a reporter gene (e.g., firefly luciferase), for easy detection. In some embodiments, can also be engineered to express a reporter gene (e.g., firefly luciferase), for easy detection. In some embodiments, NCI-H460 and NCI-H460-HLA-A2 can also be engineered to express a reporter gene (e.g., firefly luciferase), for easy detection.

In some embodiments, monitoring will be conducted by measuring tumor volume by mechanical means (caliper) and also by using in-vivo imaging systems (IVIS). In some embodiments, tumor burden can be quantified, and infiltrating T-cell populations can be analyzed by FACS.

### C. Treatment Methods

The present invention provides methods for the treatment of cancers by employing the bicistronic iCAR/aCAR constructs or monocistronic aCAR and iCAR constructs for co-transduction as described herein. The methods of treatment for cancer as described herein can employ exploiting loss of heterozygosity, or other genetic loss or allelic imbalance phenotypes found in human tumors, including, without limitation, loss of function or expression, resulting from mutations affecting one or more nucleotides (for example, without limitation, in HLA-1 genes) by means of CAR-T therapy, or by modifying other cells of the immune system.

In yet another aspect, the present invention provides a method of selecting a personalized biomarker for a subject having a tumor characterized by loss of heterozygosity, or other genetic loss or allelic imbalance phenotypes found in human tumors, the method comprising (i) obtaining a tumor biopsy from the subject; (ii) obtaining a sample of normal tissue from the subject, e.g., PBMCs; (iii) identifying a single allelic variant of a polymorphic cell surface epitope that is not expressed by cells of the tumor due to loss of heterozygosity, or other genetic loss or allelic imbalance phenotypes found in human tumors, but that is expressed by the cells of the normal tissue, thereby identifying a personalized biomarker for the subject, and (iv) determining the appropriate bicistronic iCAR/aCAR construct or monocistronic aCAR and iCAR constructs for co-transduction as described herein for use in treatment.

In a further aspect, the present invention provides a method for treating cancer in a patient having a tumor characterized by loss of heterozygosity, or other genetic loss or allelic imbalance phenotypes found in human tumors, comprising administering to the patient an effector immune cell as defined above, wherein the iCAR is directed to a single allelic variant encoding a polymorphic cell surface epitope absent from cells of the tumor due to loss of heterozygosity, or other genetic loss or allelic imbalance phenotypes found in human tumors but present at least on all cells of related mammalian normal tissue of the patient. In some embodiments, the effector immune cell comprises a bicistronic iCAR/aCAR construct as described herein.

In some embodiments, the treating results in reduced on-target, off-tumor reactivity, as compared with a treatment comprising administering to the cancer patient at least one population of immune effector cells expressing a bicistronic iCAR/aCAR construct as described herein.

In some embodiments, the population of CD4+ cells, CD8+ cells, or a combination thereof, used for treating cancer as defined above express on their surface an aCAR comprising an extracellular domain that specifically binds to a tumor-associated antigen or a non-polymorphic cell surface epitope of an antigen and an iCAR comprising an extracellular domain that specifically binds a single allelic variant of a polymorphic cell surface epitope of an antigen expressed at least in a tissue of origin of the tumor or of a housekeeping protein, which is a different antigen than that to which the extracellular domain of said aCAR binds. In some embodiments, the effector immune cell expresses the components of a bicistronic iCAR/aCAR construct or monocistronic aCAR and iCAR constructs for co-transduction as described herein.

In some embodiments, the population of CD4+ cells, CD8+ cells, or a combination thereof, used for treating cancer as defined above express on their surface an aCAR comprising an extracellular domain that specifically binds to a tumor-associated antigen or a non-polymorphic cell surface epitope of an antigen and an iCAR comprising an extracellular domain that specifically binds a single allelic variant of a polymorphic cell surface epitope of an antigen expressed at least in a tissue of origin of the tumor or of a housekeeping protein, such as an HLA genes (including for example, HLA-A, HLA-B, HLA-C, HLA-G, HLA-E, HLA-F, HLA-K, HLA-L, HLA-DM, HLA-DO, HLA-DP, HLA-DQ, or HLA-DR) which is a different antigen than that to which the extracellular domain of said aCAR binds.

In some embodiments, the population of CD4+ cells, CD8+ cells, or a combination thereof, used for treating cancer as defined above express on their surface an aCAR comprising an extracellular domain that specifically binds to a tumor-associated antigen or a non-polymorphic cell surface epitope of an antigen and an iCAR comprising an extracellular domain that specifically binds a single allelic variant of a polymorphic cell surface epitope of an antigen expressed at least in a tissue of origin of the tumor, such as an HLA-A, which is a different antigen than that to which the extracellular domain of said aCAR binds.

In some embodiments, the population of CD4+ cells, CD8+ cells, or a combination thereof, used in the method of treating cancer are selected from T cells, natural killer cells or cytokine-induced killer cells. In some embodiments, the population of CD4+ cells, CD8+ cells, or a combination thereof, comprises autologous or universal (allogeneic) effector cells. In some embodiments, the iCAR used in any one of the methods of treating cancer defined above is directed to all tissues of the patient on which the target-antigen of the aCAR is present, wherein the target antigen of the aCAR is a non-polymorphic cell surface epitope of an antigen or a single allelic variant of a polymorphic cell surface epitope is present, and said epitope is a tumor-associated antigen or is shared at least by cells of related tumor and normal tissue.

In some embodiments, the cancer is selected from the group consisting of Acute Myeloid Leukemia [LAML], Adrenocortical carcinoma [ACC], Bladder Urothelial Carcinoma [BLCA], Brain Lower Grade Glioma [LGG], Breast invasive carcinoma [BRCA], Cervical squamous cell carcinoma and endocervical adenocarcinoma [CESC], Cholangiocarcinoma [CHOL], Colon adenocarcinoma [COAD], Esophageal carcinoma [ESCA], Glioblastoma multiforme [GBM], Head and Neck squamous cell carcinoma [HNSC], Kidney Chromophobe [KICH], Kidney renal clear cell carcinoma [KIRC], Kidney renal papillary cell carcinoma [KIRP], Liver hepatocellular carcinoma [LIHC], Lung adenocarcinoma [LUAD], Lung squamous cell carcinoma [LUSC], Lymphoid Neoplasm Diffuse Large B-cell Lymphoma [DLBC], Mesothelioma [MESO], Ovarian serous cystadenocarcinoma [OV], Pancreatic adenocarcinoma [PAAD], Pheochromocytoma and Paraganglioma [PCPG], Prostate adenocarcinoma [PRAD], Rectum adenocarcinoma [READ], Sarcoma [SARC], Skin Cutaneous Melanoma [SKCM], Stomach adenocarcinoma [STAD], Testicular Germ Cell Tumors [TGCT], Thymoma [THYM], Thyroid carcinoma [THCA], Uterine Carcinosarcoma [UCS], Uterine Corpus Endometrial Carcinoma [UCEC], Uveal Melanoma [UVM], Non-small cell lung carcinoma [NSCLC], and Small cell lung cancer [SCLC].

In some embodiments, the bicistronic iCAR/aCAR construct or monocistronic aCAR and iCAR constructs for co-transduction for use in the treatment of cancer is any bicistronic iCAR/aCAR construct described herein. In some embodiments, the bicistronic iCAR/aCAR construct used to treat the cancer, such as any one of the cancer types recited above, is directed against or specifically binds to a single allelic variant of an HLA genes (including for example, HLA-A, HLA-B, HLA-C, HLA-G, HLA-E, HLA-F, HLA-K, HLA-L, HLA-DM, HLA-DO, HLA-DP, HLA-DQ, or HLA-DR, HLA-B gene or HLA-C gene or against a single allelic variant. In some embodiments, the treatment method employs administration of a population of CD4+ cells, CD8+ cells, or a combination thereof, comprising the bicistronic iCAR/aCAR construct or monocistronic aCAR and iCAR constructs for co-transduction. In some embodiments, the treatment method employs administration of a population of CD4+ cells, CD8+ cells, or a combination thereof, expressing the bicistronic iCAR/aCAR construct or monocistronic aCAR and iCAR constructs for co-transduction.

In some embodiments, the bicistronic iCAR/aCAR or monocistronic aCAR and iCAR constructs for co-transduction for use in the treatment of cancer comprises an amino acid sequence encoded by a nucleic acid sequence selected from the group consisting of SEQ ID NO:1, SEQ ID NO:3, SEQ ID NO:5, SEQ ID NO:7, SEQ ID NO:9, SEQ ID NO:11, SEQ ID NO:13, SEQ ID NO:15, SEQ ID NO:17, SEQ ID NO:19, SEQ ID NO:21, SEQ ID NO:23, SEQ ID NO:25, SEQ ID NO:27, SEQ ID NO:29, SEQ ID NO:31, SEQ ID NO:33, SEQ ID NO:35, SEQ ID NO:275, SEQ ID NO:277, SEQ ID NO:279, SEQ ID NO:281, SEQ ID NO:321, SEQ ID NO:323, and SEQ ID NO:325. In some embodiments, the bicistronic iCAR/aCAR construct or monocistronic aCAR and iCAR constructs for co-transduction comprises an amino acid sequence encoded by a nucleic acid sequence selected from the group consisting of SEQ ID NO:275, SEQ ID NO:277, SEQ ID NO:279, SEQ ID NO:281, SEQ ID NO:321, SEQ ID NO:323, and SEQ ID NO:325. In some embodiments, the treatment method employs administration of a population of CD4+ cells, CD8+ cells, or a combination thereof, comprising the bicistronic iCAR/aCAR construct or monocistronic aCAR and iCAR constructs for co-transduction. In some embodiments, the treatment method employs administration of a population of CD4+ cells, CD8+ cells, or a combination thereof, expressing the bicistronic iCAR/aCAR construct or monocistronic aCAR and iCAR constructs for co-transduction.

In some embodiments, the bicistronic iCAR/aCAR for use in the treatment of cancer comprises an amino acid sequence selected from the group consisting of SEQ ID NO:2, SEQ ID NO:4, SEQ ID NO:6, SEQ ID NO:8, SEQ ID NO:10, SEQ ID NO:12, SEQ ID NO:14, SEQ ID NO:16, SEQ ID NO:18, SEQ ID NO:20, SEQ ID NO:22, SEQ ID NO:24, SEQ ID NO:26, SEQ ID NO:28, SEQ ID NO:30, SEQ ID NO:32, SEQ ID NO:34, SEQ ID NO:36, SEQ ID NO:276, SEQ ID NO:278, SEQ ID NO:280, SEQ ID NO:282, SEQ ID NO:322, SEQ ID NO:324, and SEQ ID NO:326. In some embodiments, the bicistronic iCAR/aCAR construct or monocistronic aCAR and iCAR constructs for co-transduction comprises an amino acid sequence selected from the group consisting of SEQ ID NO:276, SEQ ID NO:278, SEQ ID NO:280, SEQ ID NO:282, SEQ ID NO:322, SEQ ID NO:324, and SEQ ID NO:326. In some embodiments, the treatment method employs administration of a population of CD4+ cells, CD8+ cells, or a combination thereof, comprising the bicistronic iCAR/aCAR construct or monocistronic aCAR and iCAR constructs for co-transduction. In some embodiments, the treatment method employs administration of a population of CD4+ cells, CD8+ cells, or a combination thereof, expressing the bicistronic iCAR/aCAR construct or monocistronic aCAR and iCAR constructs for co-transduction.

The compositions may be formulated for parenteral administration by injection, e.g., by bolus injection or continuous infusion. Formulations for injection may be presented in unit dosage form, e.g., in ampoules or in multidose containers, with an added pharmaceutically acceptable carrier and/or preservative. The compositions may take such forms as suspensions, solutions or emulsions in oily or aqueous vehicles, and may contain formulatory agents such as suspending, stabilizing and/or dispersing agents.

For purposes of clarity, and in no way limiting the scope of the teachings, unless otherwise indicated, all numbers expressing quantities, percentages or proportions, and other numerical values recited herein, should be interpreted as being preceded in all instances by the term “about.” Accordingly, the numerical parameters recited in the present specification are approximations that may vary depending on the desired outcome. For example, each numerical parameter may be construed in light of the number of reported significant digits and by applying ordinary rounding techniques.

### EXAMPLES

### Example 1. Evaluation of CD4 Vs. CD8 Car T Cells Comprising Bicistronic Inhibitory Chimeric Antigen Receptor (iCAR)/Activating Chimeric Antigen Receptor (aCAR) Constructs

### Introduction

This example provides the results related to evaluation of CD4 and CD8 CAR T cells comprising bicistronic inhibitory chimeric antigen receptor (iCAR)/activating chimeric antigen receptor (aCAR) constructs in order to develop cancer therapeutics for use in safely targeting tumors that have lost genomic segments encoding cell-membrane proteins with polymorphic protein coding changes). Data provided in the example and figures include evaluation of purity and CAR expression of CD4 CAR T cells, evaluation of CD4 and CD8 CAR T cells for efficacy and protection, and evaluation of CD4 and CD8 CAR T cells for cytokine secretion.

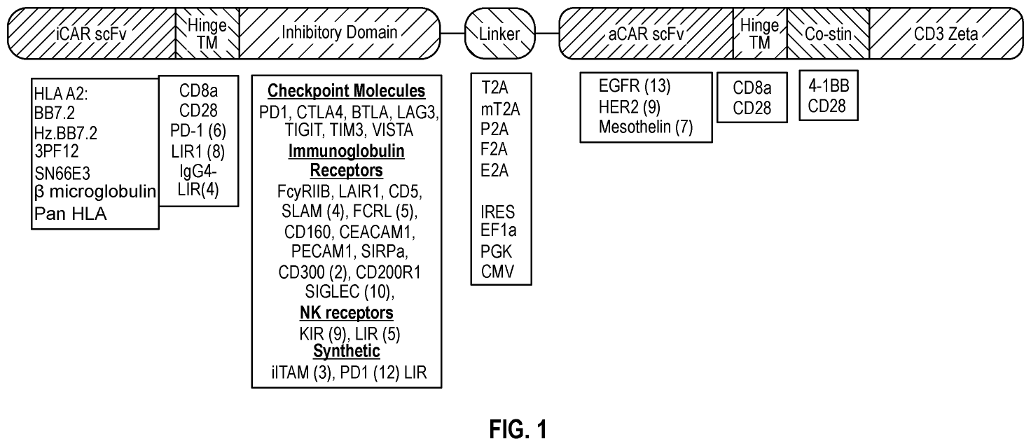

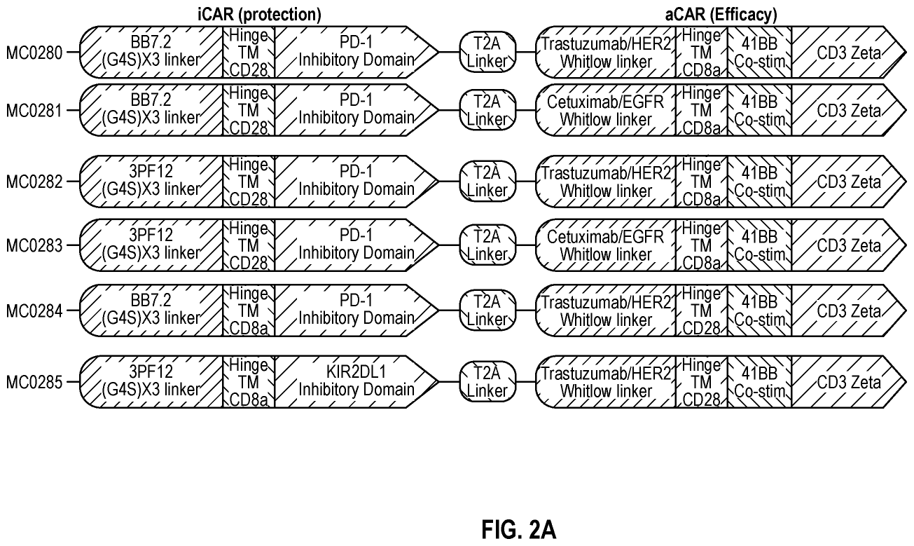

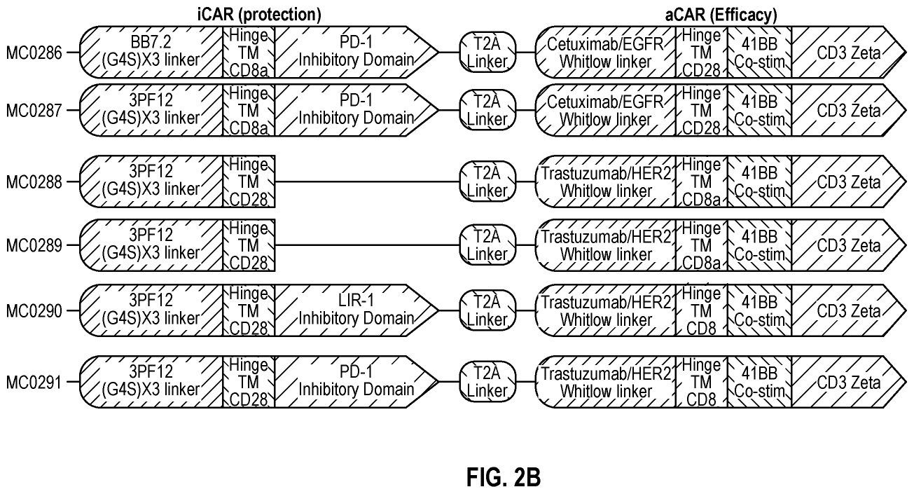

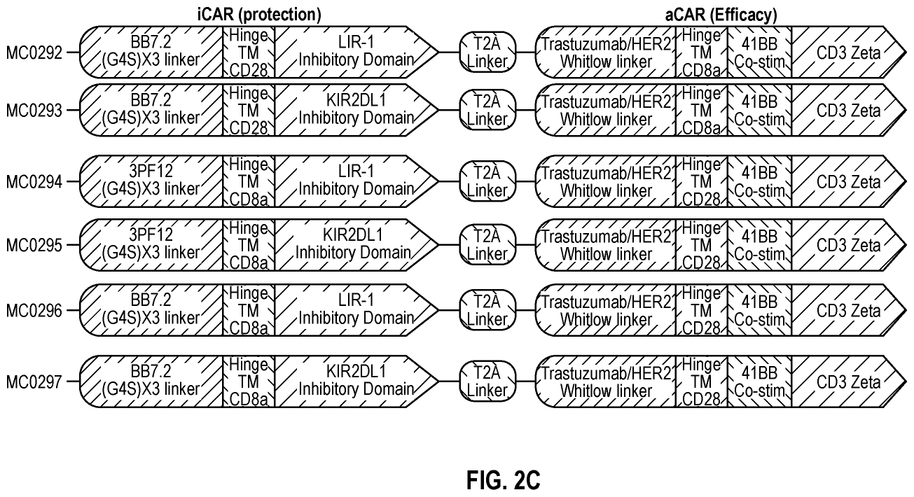

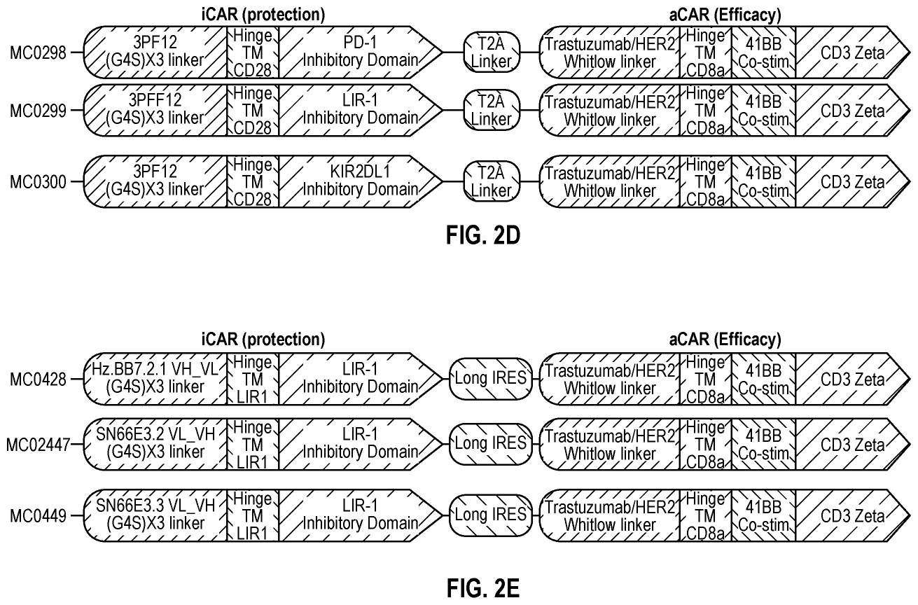

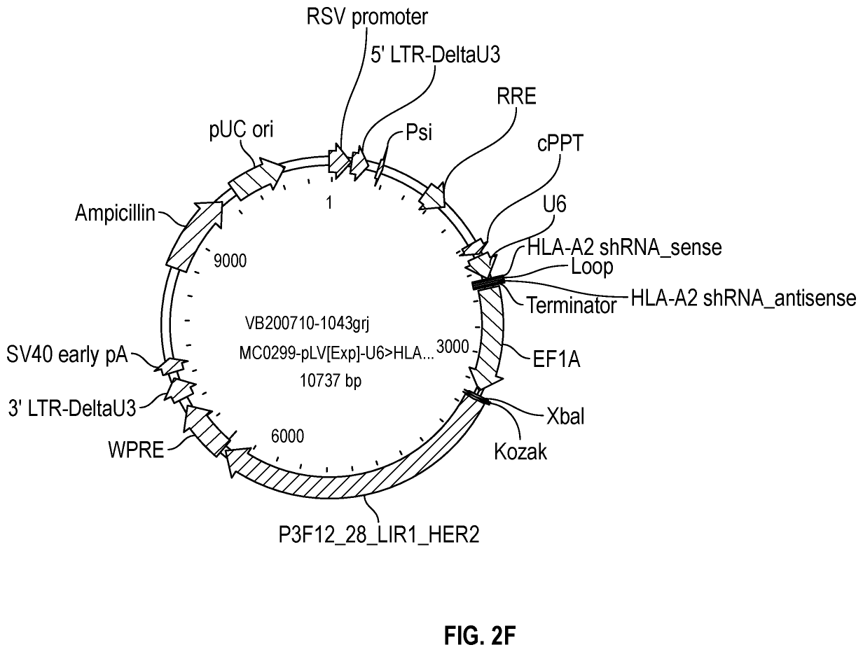

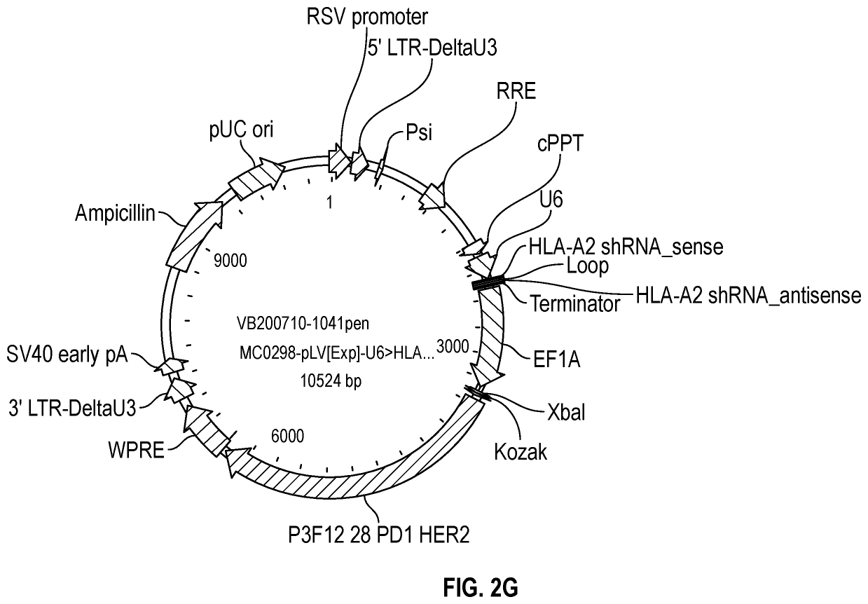

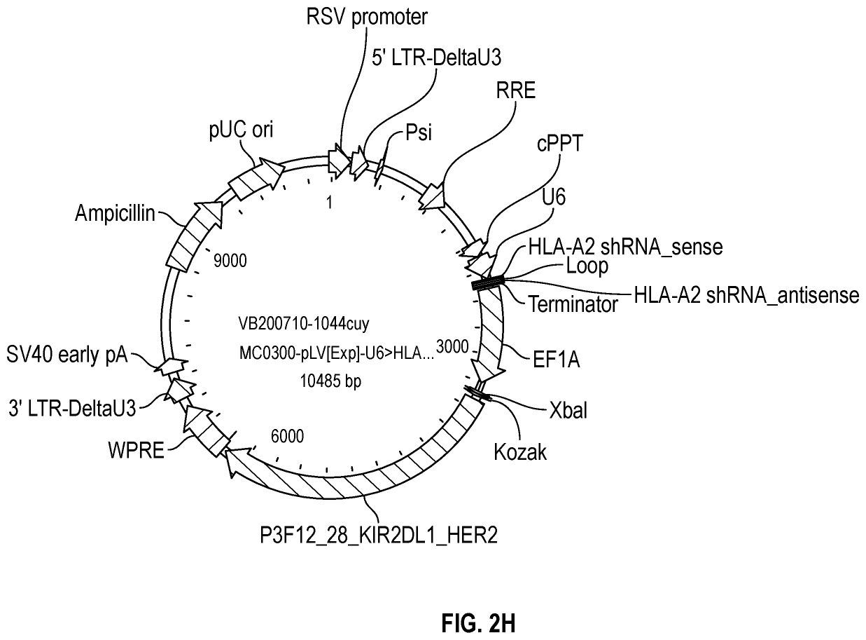

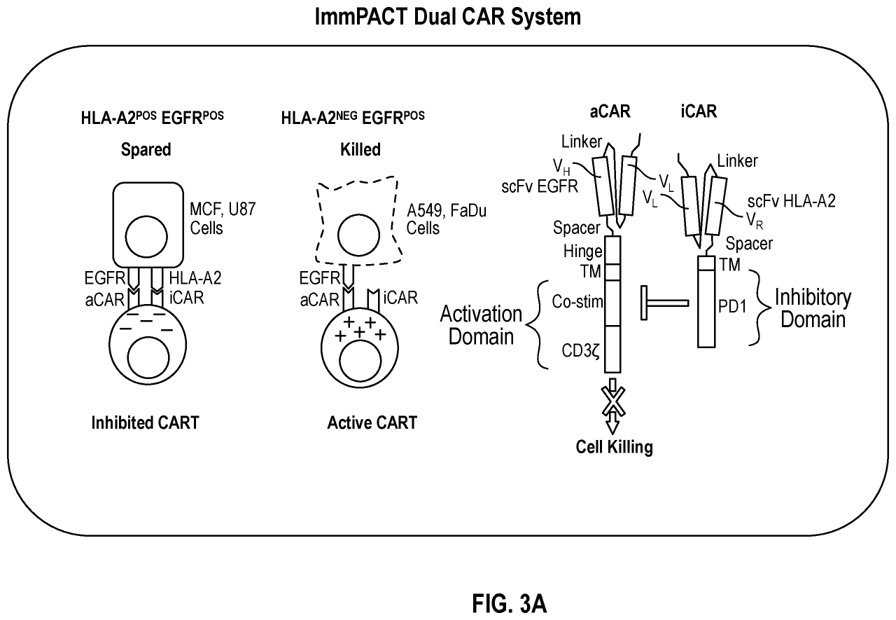

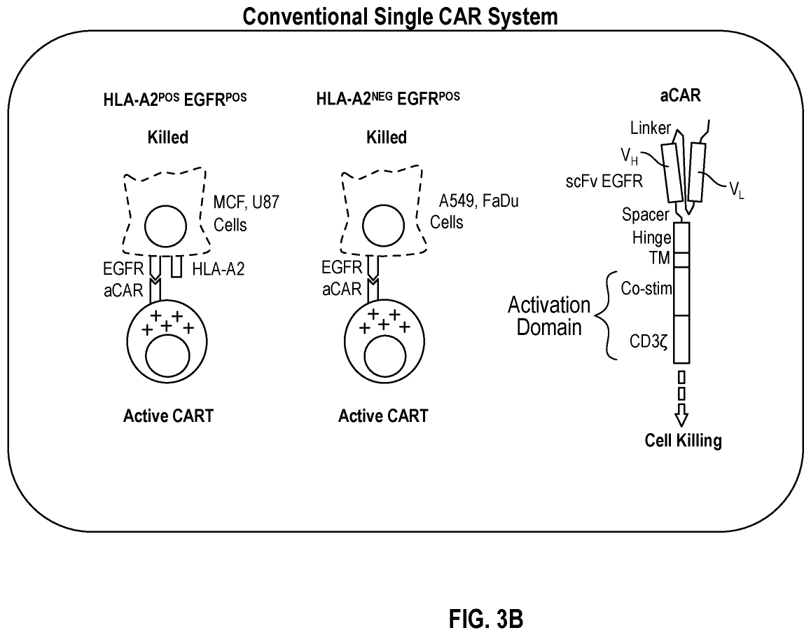

CD4 CAR T cells were validated for efficacy and conferred complete protection. These findings were consistent across different donors; iCAR scFv, hinge and transmembrane domains, and iDomain; isolation time (day 0, day 14); fresh and thawed effector cells and several target cell lines. See FIGS. 1-22 as well as Tables 1-22 for illustrative design and evaluation of examples of iCAR and aCAR constructs as described herein, as well as sequences thereof, in unsorted/untouched, CD4, and CD8 cells.

### Materials and Methods

### PBMC Purification

Leukocyte enriched samples were acquired from The Sheba Medical Center blood bank, diluted with equal volumes of PBS and loaded on Ficoll-Paque PLUS (GE Healthcare) for density-based cell separation. Preparation was according to manufacturer's protocol. Mononuclear cells were collected from the plasma/Ficoll interface, washed several times and resuspended in Cryostor CS10 (Merck).

### EasySep™ Human CD4+ T Cell Isolation

Primary blood mononuclear cells (PBMCs) were thawed and viable cells enumerated using a Countess Automated Cell Counter (Invitrogen). The PBMC sample was prepared within the volume range of 0.5-2 mL at a cell concentration of 5.0E+07 cells/mL in a 5 mL polystyrene round-bottom tube. EasySep™ Dextran EasySep™ Direct Human CD4+ T Cell Isolation Cocktail (StemCell Technologies) was added to the cells at a concentration of 50 μL/mL of sample, mixed and incubated for 5 minutes at room temperature. RapidSpheres™ (StemCell Technologies) were then vortexed for 30 seconds until particles appeared evenly dispersed, then added to the sample at 50 μL/mL of sample and mixed. The tube was placed into the EasyEights™ magnet and incubated for 10 minutes at room temperature. The CD4+ T cell enriched cell suspension was then carefully pipetted into a new 5 mL polystyrene round-bottom tube and placed into the magnet and incubated for 10 minutes at room temperature for a second separation. The enriched cell suspension was carefully pipetted into a new 5 mL polystyrene round-bottom tube. Isolated cells were ready for use.

### EasySep™ Human CD8+ T Cell Isolation

Primary blood mononuclear cells (PBMCs) were thawed and viable cells enumerated using a Countess Automated Cell Counter (Invitrogen). The PBMC sample was prepared within the volume range of 0.5-2 mL at a cell concentration of 5.0E+07 cells/mL in a 5 mL polystyrene round-bottom tube. EasySep™ Dextran EasySep™ Direct Human CD8+ T Cell Isolation Cocktail (StemCell Technologies) was added to the cells at a concentration of 50 μL/mL of sample, mixed and incubated for 5 minutes at room temperature. RapidSpheres™ (StemCell Technologies) were then vortexed for 30 seconds until particles appeared evenly dispersed, then added to the sample at 50 μL/mL of sample and mixed. The tube was placed into the EasyEights™ magnet and incubated for 10 minutes at room temperature. The CD8+ T cell enriched cell suspension was then carefully pipetted into a new 5 mL polystyrene round-bottom tube and placed into the magnet and incubated for 10 minutes at room temperature for a second separation. The enriched cell suspension was carefully pipetted into a new 5 mL polystyrene round-bottom tube. Isolated cells were ready for use.

### PBMC Culture and Transduction

PBMCs were thawed and seeded at a density of 1×10⁶ cells/ml in LymphoOne medium (Takara-Bio, Kusatsu, Japan) supplemented with 100 U/ml IL2 (Miltenyi Biotech, Bergisch Gladbach, Germany). The next day concentrated lentiviruses were added at an MOI of 5, 10, or 20 (according to prior calibrations). After 3 days cells were transferred to 24-well G-Rex plates (Wilson Wolf, Saint Paul, MN) containing LymphoOne medium supplemented with 1% human serum (Access Biologicals, Vista, CA) and 100 U/ml IL2. On day 7 post-thaw 100 U/ml IL2 was added, and on day 8 the medium was replaced. Functional assays were typically performed.

### ELISA

Target cells expressing nuclear-GFP (nGFP) were seeded in 96 well plates (Thermo, NU-167008), 5×10³ cells per well, in LymphoOne medium supplemented with 1% human serum. The next day, transduced or electroporated PBMCs were added to the wells at 5:1 E:T ratio. Cells are co-incubated for 15-18 hrs at 37C, 5% CO₂. Following co-incubation, supernatant is harvested and transferred to non-binding 96-well plates (Greiner, #655901) at −200c. Supernatants are diluted 3 and 100-fold, ELISA performed as to manufactures instruction (Human IFN-gamma Quantikine, R&D, #SIF50) and quantified using Tecan plate reader.

### Quantification of Antigen Expression by Flow Cytometry

The MESF/“Antibody Binding Capacity” (ABC) ratio of a particular antibody can be used to quantify the number of antigen sites per cell. To establish the MESF/ABC ratio of each antibody Lot, MFIs of stained SCQ beads were correlated to the MFIs of MESF standards. The slope of the curve constitutes the ratio of fluorochrome label in MESF units per antibody. The MESF/ABC of every antibody Lot was measured using mouse/human/rat Simple Cellular Quantum (SCQ) Beads and MESF standards purchased from Bangs laboratories. Each of the 4 populations of SCQ beads has a known Antibody Binding Capacity (ABC), typically in the range of several thousands to 500-800K, so by staining these beads with an antibody at near saturation, one can correlate the fluorescence measurement (MFI) on a flow cytometer to the amount of bound antibody (ABC). MESF standard beads are composed of 4-5 different bead populations labeled with a known amount of fluorochrome molecules. By running MESF beads on a flow cytometer, one can correlate an MFI measurement to MESF units and compare between data that was collected on multiple different occasions, PMT voltages and instruments. When using HLA-A2/NYESO1-PE tetramers to stain tag-less iCAR constructs, the MESF/ABC ratio was established by staining control Jurkat cell lines that express a tagged aCAR and iCAR at high and low levels, with both quantifiable Anti-Myc Tag antibody and HLA-A2/NYESO1-PE tetramers. For each staining 100-200K positive cells were washed twice with 100 ul of cold FACS buffer (2% FCS in PBS ×1) by centrifugation, 300g for 5 min at 4° C. For Flag tagged aCAR and Myc tagged iCAR quantification, the cells were stained with 50 ul of APC (130-119-584, Miltenyi) and FITC (130-116-485, Miltenyi) labeled antibodies diluted 1/25 with FACS buffer. For un-tagged trastuzumab aCAR and Anti-HLA-A2 iCAR quantification, primary human Anti-Trastuzumab scFv69 (Ab00618-10.0, Absolute Antibody), HLA-A2/NYESO1-PE tetramers (TB-M105-1, MBL) and secondary Anti-human Fc APC (BLG-409306, biolegend) were diluted in FACS buffer, 1/25, 1/5 and 1/10 respectively. For target cell line antigen quantification, Anti-EGFR PE (FAB9577P-100, R&D), Anti-HER2 APC (130-106-696, Miltenyi) and Anti-HLA-A2 APC (17-9876-42, ebioscience) were diluted with FACS buffer, 1/2.5, 1/10 and 1/5 respectively. The cells were incubated at 4° C. in the dark for 45-60 min and washed thrice with 100 ul cold FACS buffer as described previously. The cells were resuspended with 150 ul of FACS buffer or PBS ×1 containing 0.5-1 ug/ml DAPI (MBD0015-1, Merck-Sigma). The cells were analyzed by flow cytometry (BD FACS Celesta or MACSQuant Analyzer 10) collecting 10K-50K double positive events from each sample. Next, without changing the PMT voltages on the instrument, 5-10K events of each population of relevant MESF standard beads (FITC 555P-5ML, APC 823-5ML, PE 827-5ML, Bangs), were collected. FlowJo software was used to gate and calculate MFIs (Geometric Mean Fluorescence) and MESF beads QuickCal files, provided by the manufacturer, were used to convert the MFIs in to MESF units. Next, the values were converted to ABC units Using the MESF/ABC curves of the specific antibody lots used.

### Discussion

### Evaluation of CD4 Cells for Efficacy and Protection

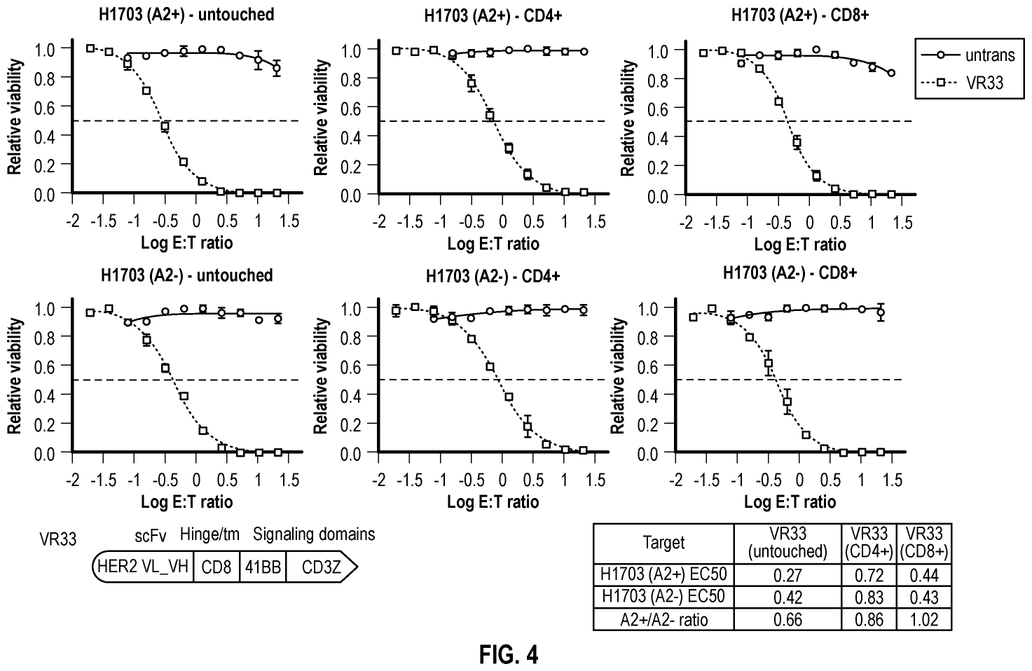

The efficacy of untouched, CD4, and CD8 CAR T cells comprising a VR33 aCAR construct was evaluated in FIG. 4. The results demonstrated that efficacy of the CAR T cells was maintained between CD4+ and CD8+ cells.

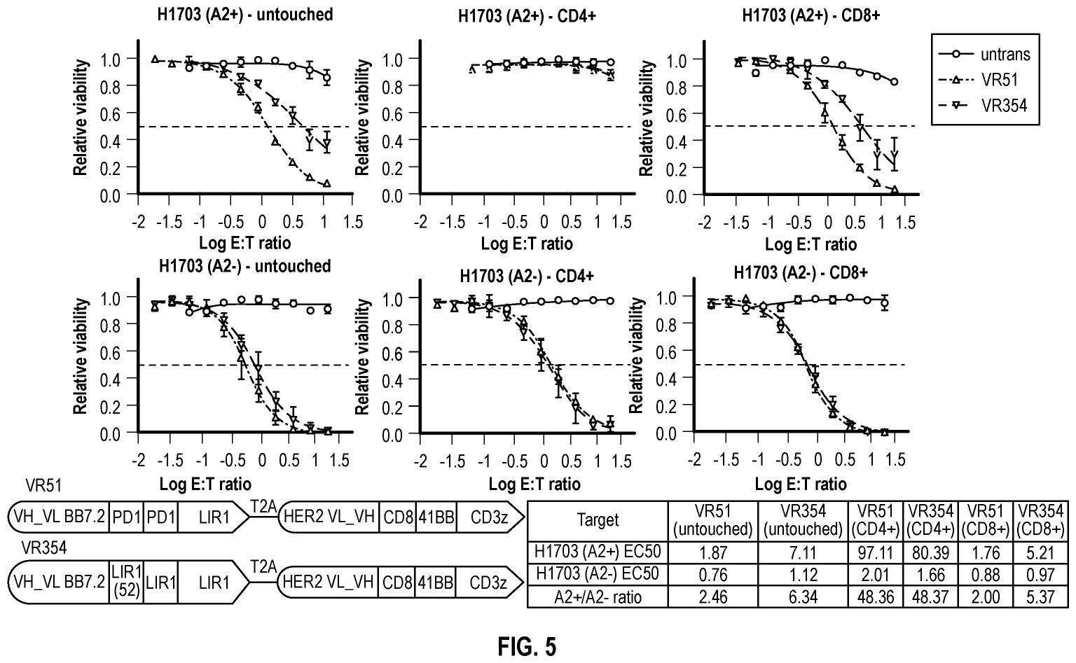

The protection and efficacy untouched, CD4, and CD8 CAR T cells comprising VR51 and VR354 bicistronic iCAR/aCAR constructs was evaluated in FIG. 5. The results demonstrated that protection was complete for CD4+ cells.

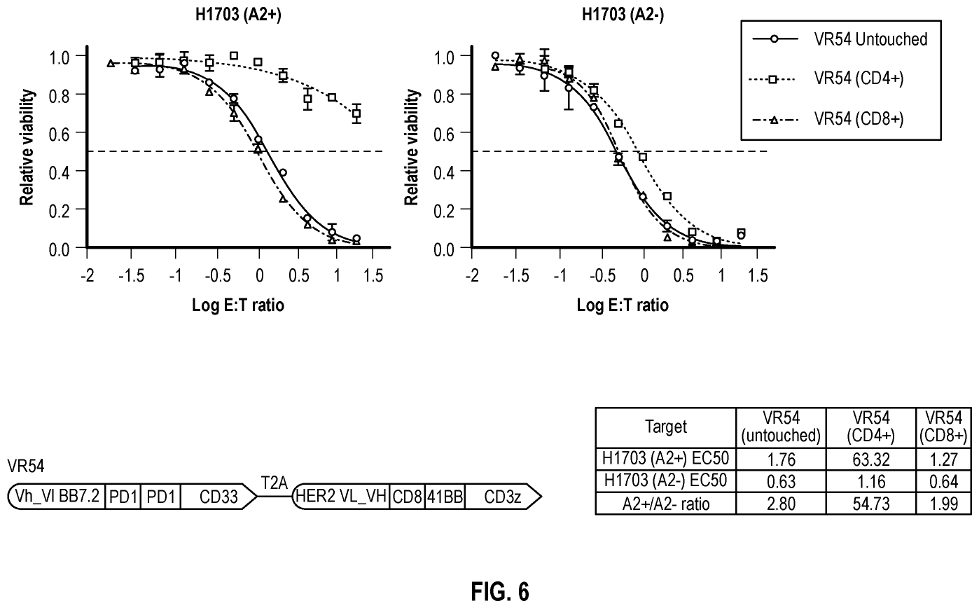

The efficacy and protection of thawed untouched, CD4, and CD8 CAR T cells comprising a VR54 bicistronic iCAR/aCAR construct was evaluated in FIG. 6. The results demonstrated that protection was complete for thawed CD4+ cells, and efficacy of thawed CD4+ cells was maintained.

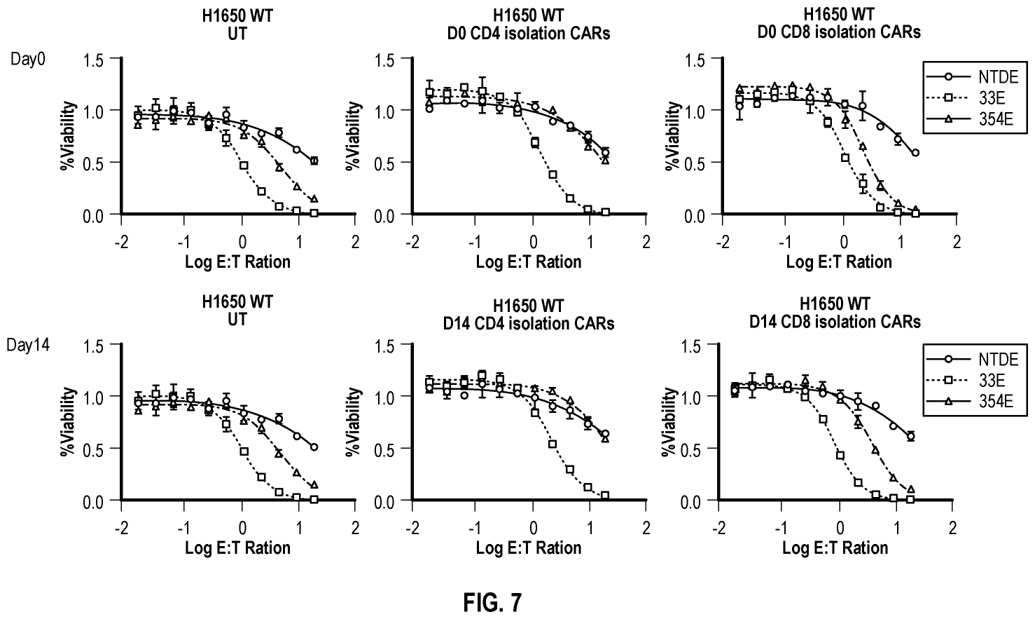

The efficacy of untouched (UT), CD4, and CD8 CAR T cells isolated on Day 0 or Day 14 and comprising a VR33 aCAR construct (33E) or a VR354 bicistronic iCAR/aCAR construct (354E) was evaluated in FIG. 7. The results demonstrated that efficacy of the CAR T cells isolated on Day 0 and on Day 14 was similar for each of untouched, CD4+, and CD8+ cells.

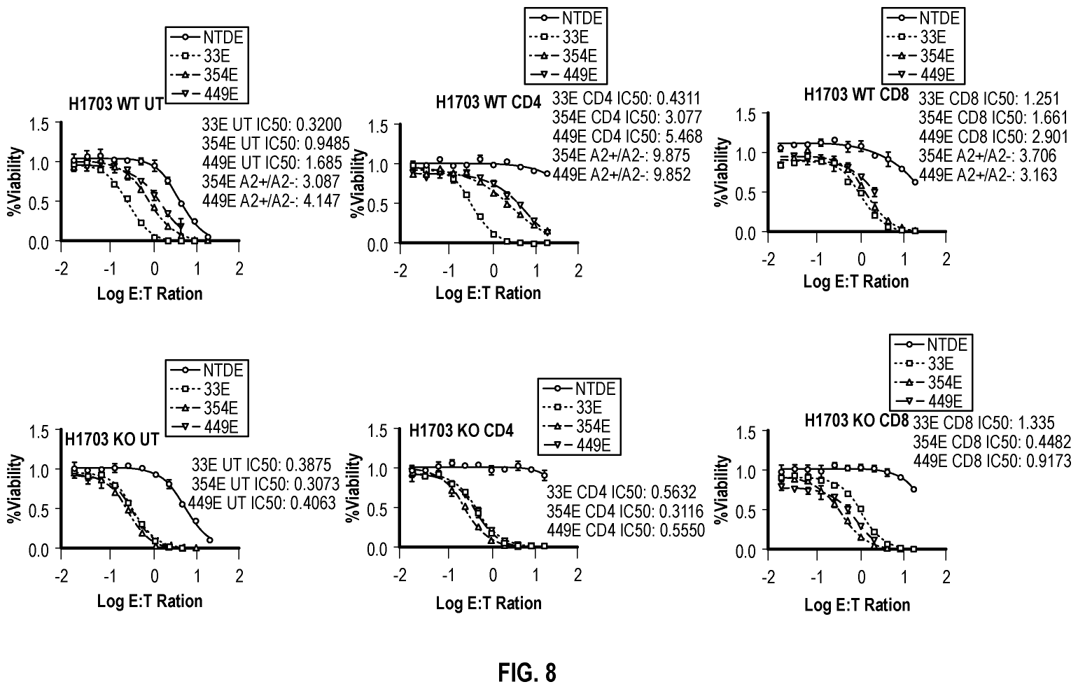

The protection and efficacy of untouched, CD4, and CD8 CAR T cells comprising a VR33 aCAR construct (33E), a VR354 bicistronic iCAR/aCAR construct (354E), or a VR449 bicistronic iCAR/aCAR construct (449E) was evaluated in FIG. 8. The results demonstrated that the augmented CD4 protection was independent of the iCAR scFv tested.

### Evaluation of Purity and CAR Expression of CD4 and CD8 Isolated Cells

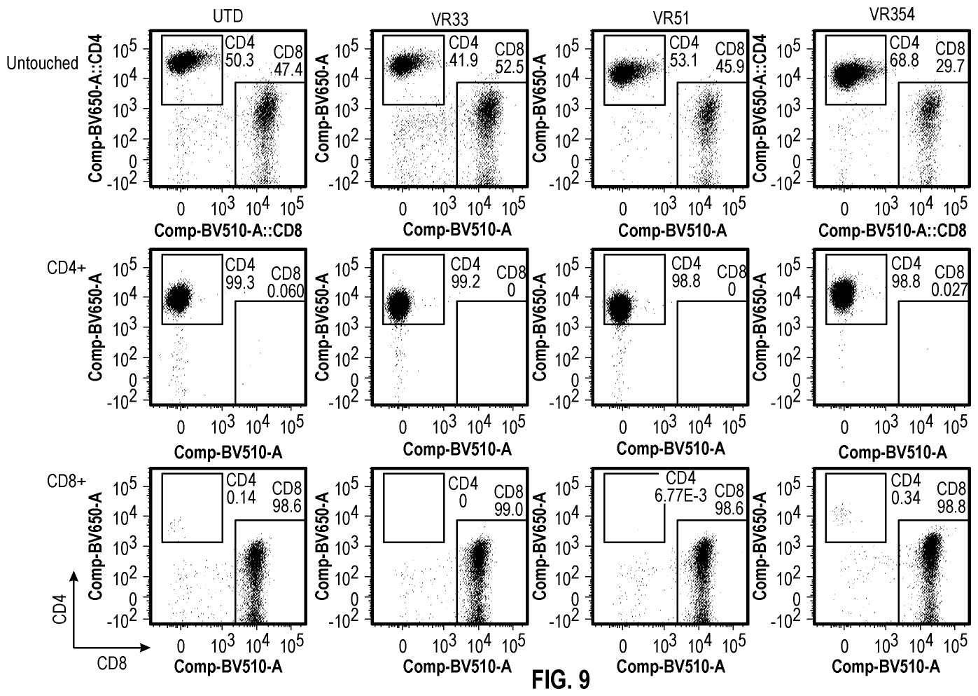

FACS analysis of CD4+ CAR T cells following negative selection is shown in FIG. 9. The FACS analysis showed that the purity of CD4+ CAR T cells was very high following negative selection, but the yield was low.

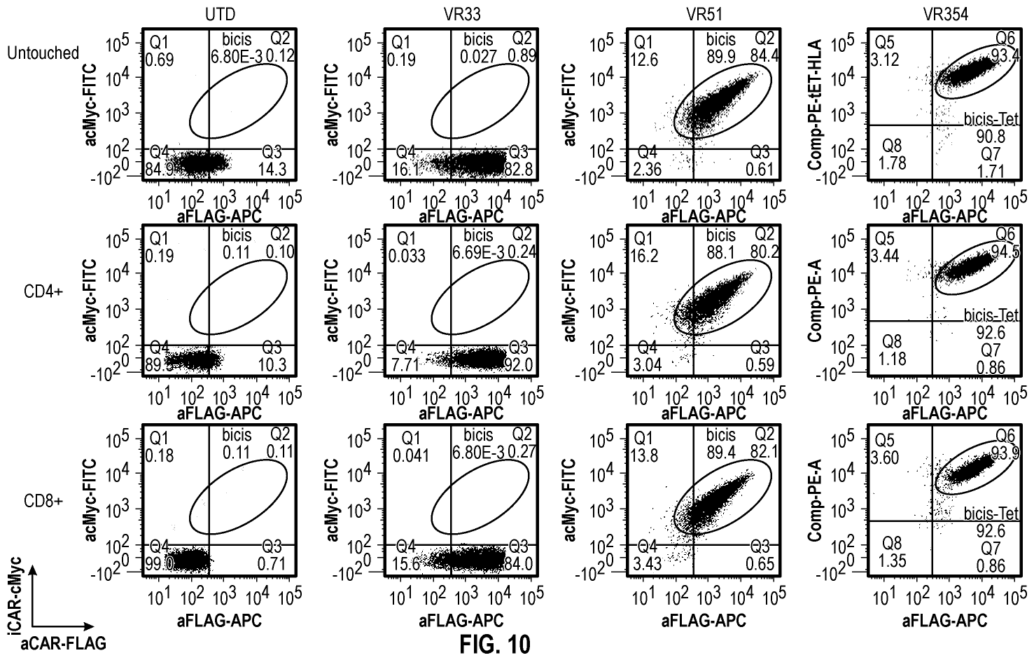

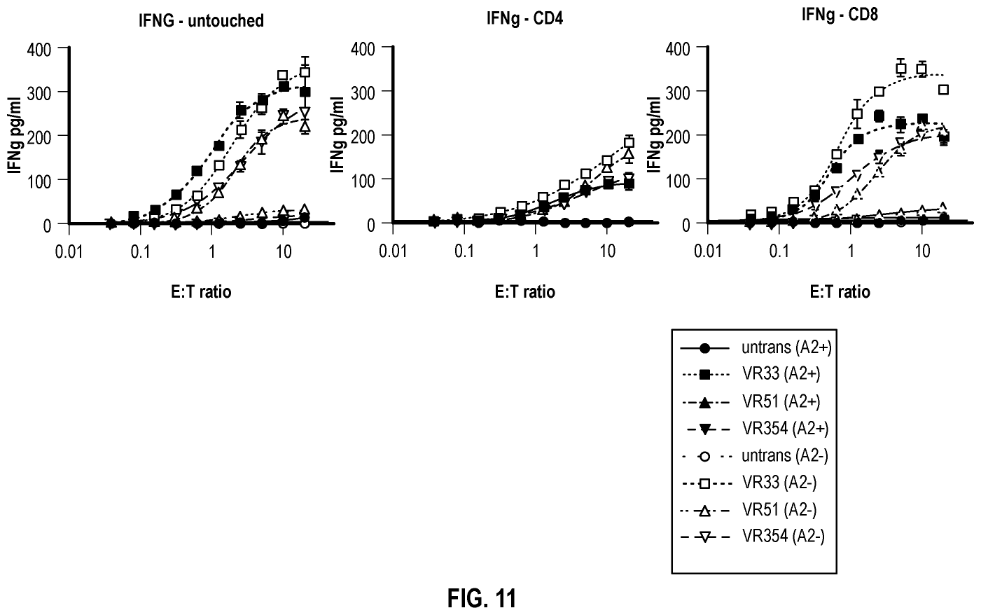

FACS analysis of aCAR-iCAR expression in isolated CD4 and CD8 cells is shown in FIG. 10. The FACS analysis showed that the aCAR-iCAR expression was similar for untouched CAR T cells, isolated CD4+ CAR T cells, and isolated CD8+ CAR T cells.

### Evaluation of Cytokine Secretion in CD4 and CD8 Isolated Cells

IFNg secretion of untouched, CD4, and CD8 CAR T cells was analyzed in FIG. 11. The results demonstrated that CD8+ CAR T cells secreted higher levels of IFNg compared to CD4 cells, and that the protection was nearly to background. They also showed that the VR33 aCAR produced more IFNg than either of the bicistronic iCAR/aCAR constructs VR51 or VR354 in response to H1703 A2−.

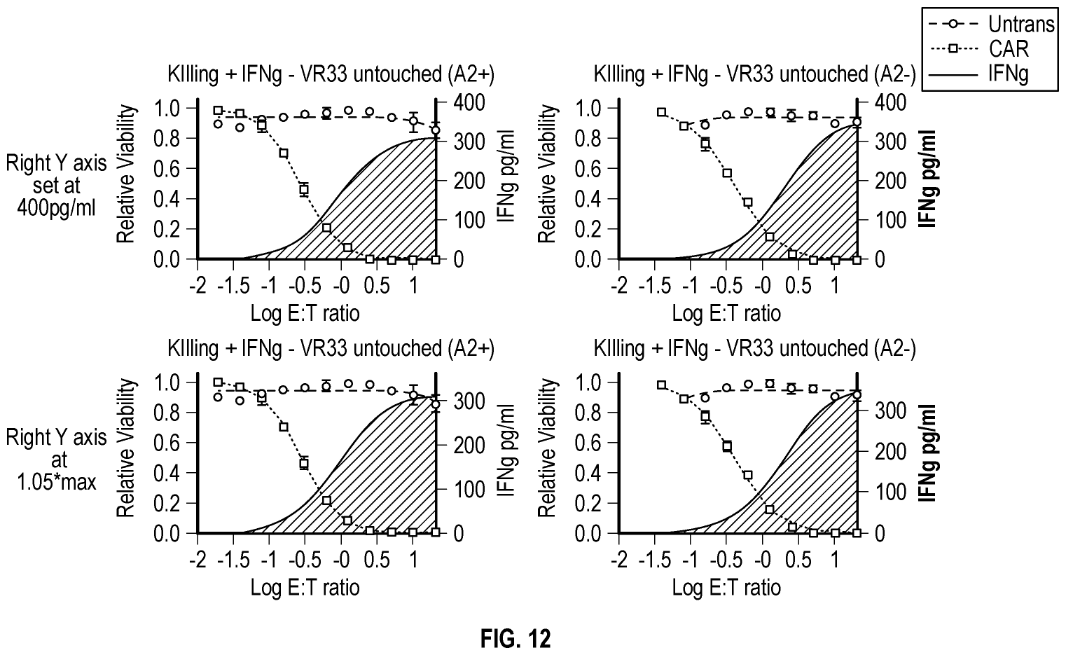

Killing and IFNg secretion of A2+ and A2− cells by VR33 untouched cells was analyzed in FIG. 12. The results show that there was a clear relationship between killing and IFNg secretion, as the killing and IFNg secretion increased at similar effector versus target ratios.

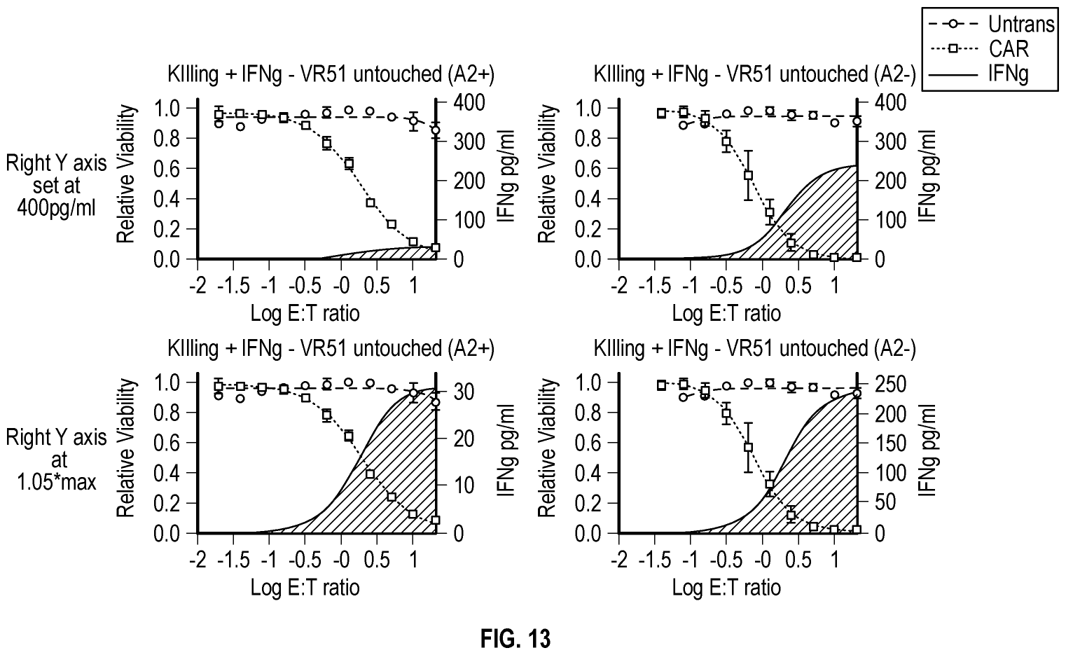

Killing and IFNg secretion of A2+ and A2− cells by VR51 untouched cells was analyzed in FIG. 13. The results show that there was a clear relationship between killing and IFNg secretion, as the killing and IFNg secretion increased at similar effector versus target ratios. They also show that there was low IFNg to A2+, and that the protection of IFNg to H1703 A2+ was strong but not complete.

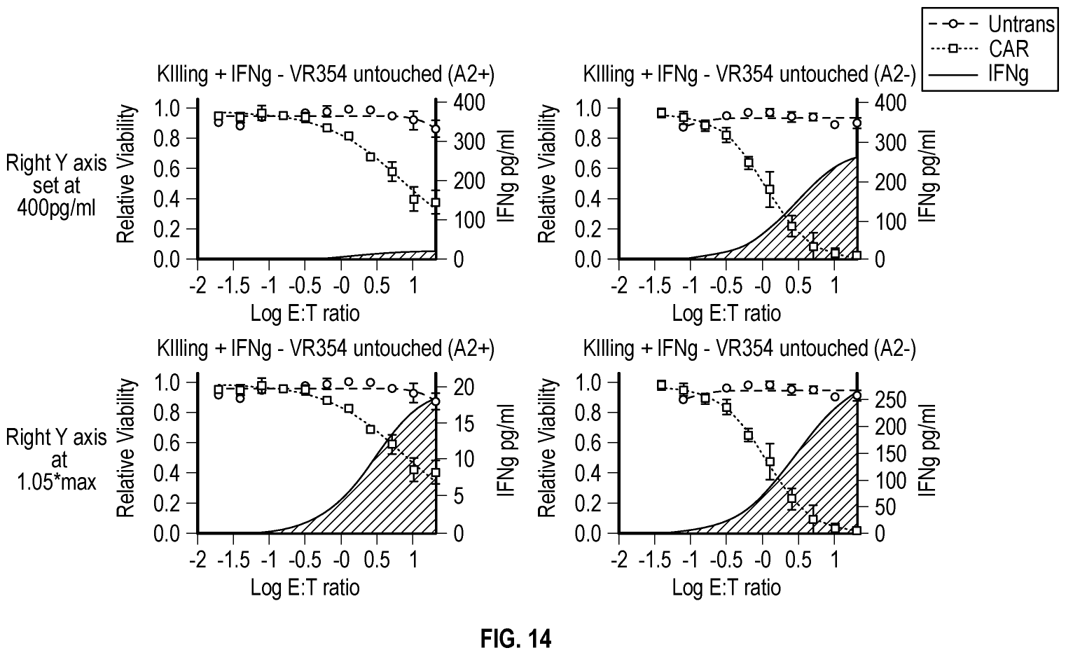

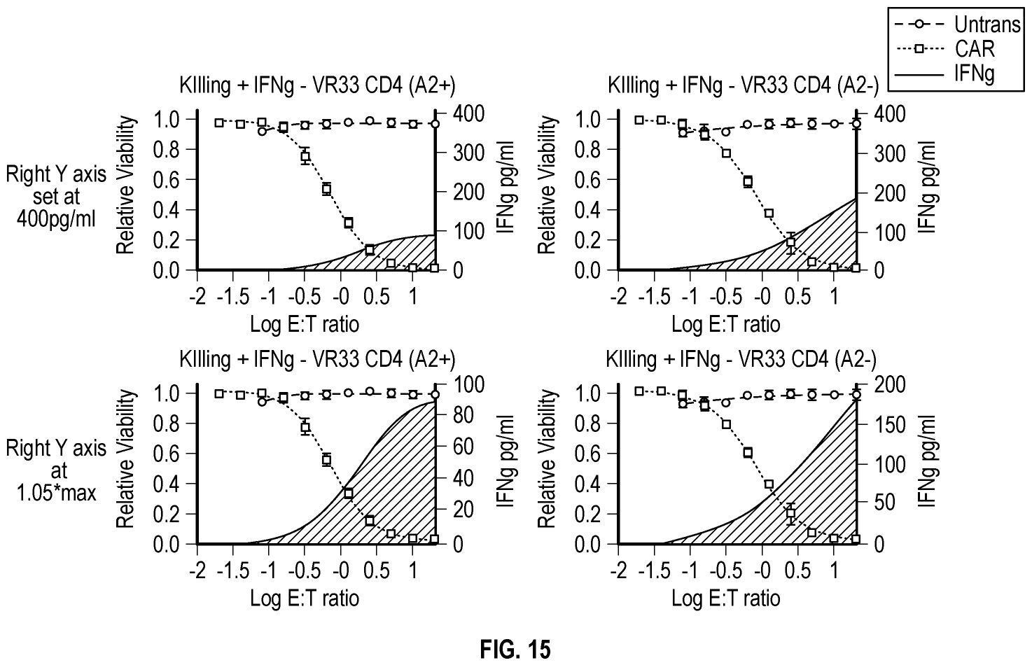

Killing and IFNg secretion of A2+ and A2− cells by VR354 untouched cells was analyzed in FIG. 14. The results show that there was a clear relationship between killing and IFNg secretion, as the killing and IFNg secretion increased at similar effector versus target ratios. They also show that there was low IFNg to A2+, and that the protection of IFNg to H1703 A2+ was strong but not complete.

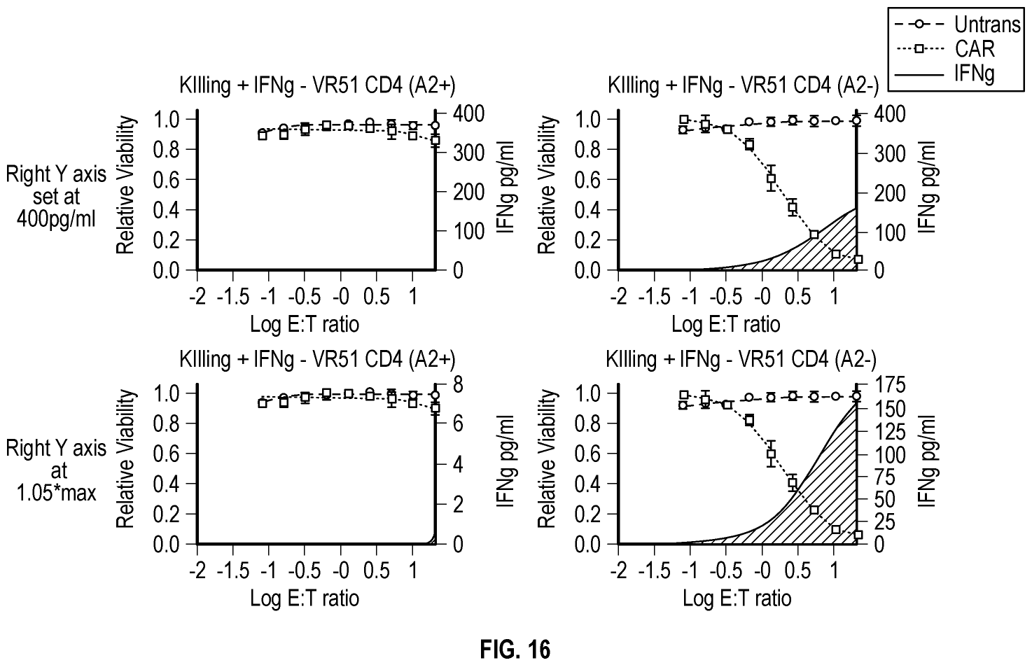

Killing and IFNg secretion of A2+ and A2− cells by VR33 CD4 cells was analyzed in FIG. 15. The results show that there was a clear relationship between killing and IFNg secretion, as the killing and IFNg secretion increased at similar effector versus target ratios. They also show that IFNg production was lower in CD4 cells than in untouched.

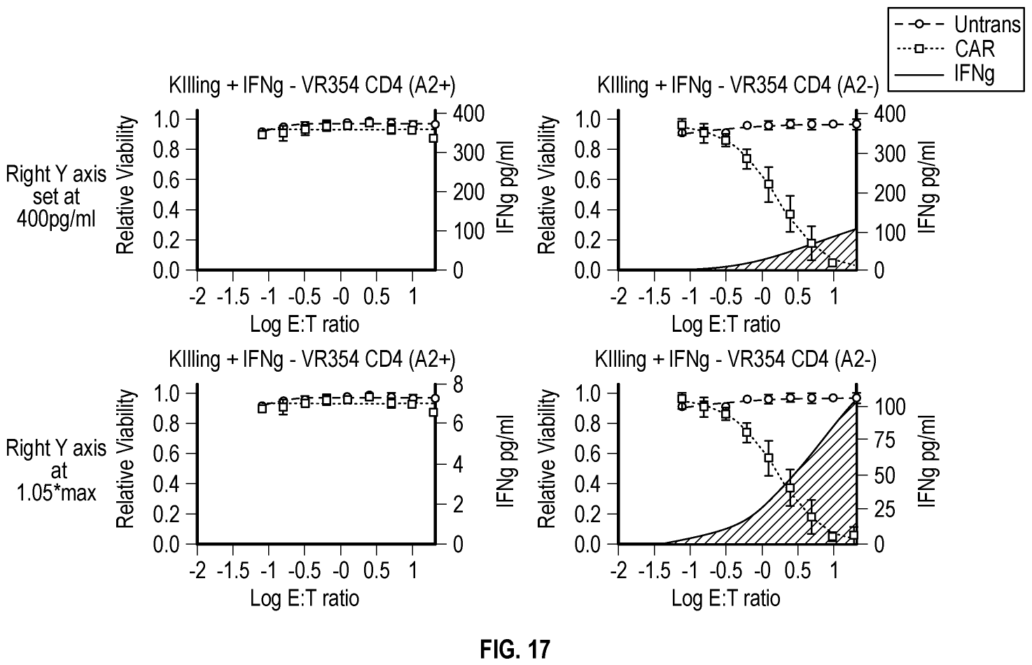

Killing and IFNg secretion of A2+ and A2− cells by VR51 CD4 cells was analyzed in FIG. 16. The results show that there was a clear relationship between killing and IFNg secretion, as the killing and IFNg secretion increased at similar effector versus target ratios. They also show that there was no observable IFNg to H1703 A2+(complete protection).

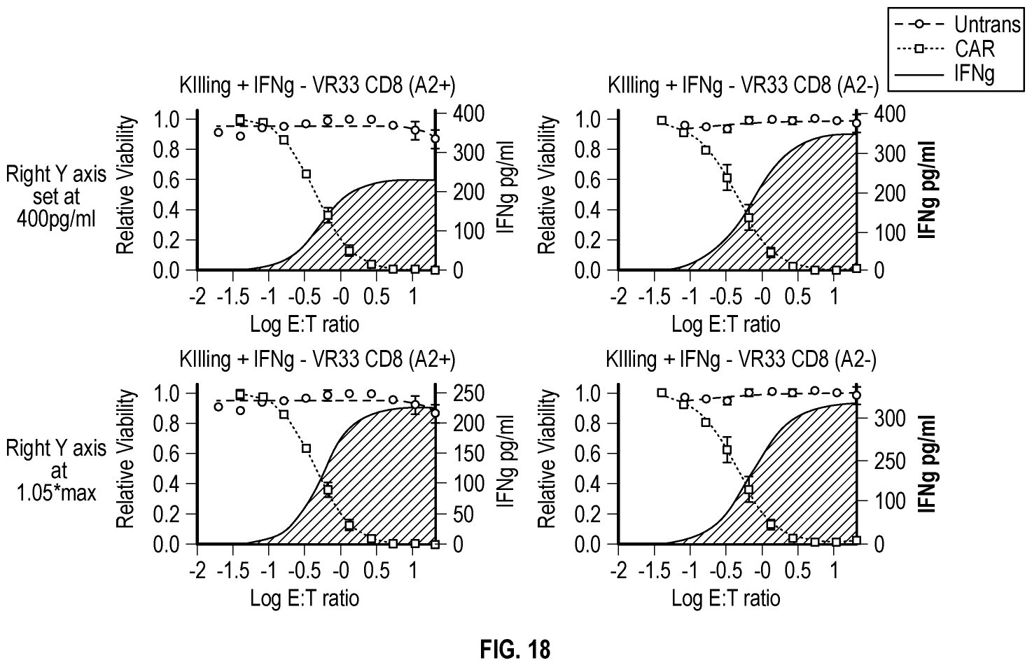

Killing and IFNg secretion of A2+ and A2− cells by VR354 CD4 cells was analyzed in FIG. 17. The results show that there was a clear relationship between killing and IFNg secretion, as the killing and IFNg secretion increased at similar effector versus target ratios. They also show that there was no observable IFNg to H1703 A2+(complete protection).

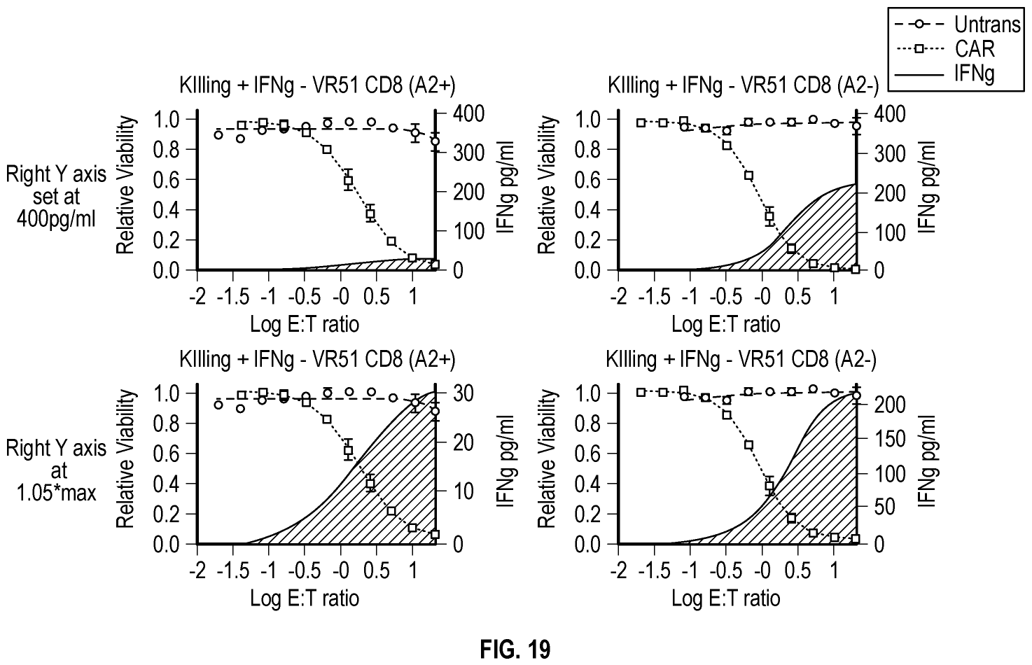

Killing and IFNg secretion of A2+ and A2− cells by VR33 CD8 cells was analyzed in FIG. 18. The results show that there was a clear relationship between killing and IFNg secretion, as the killing and IFNg secretion increased at similar effector versus target ratios. They also show that IFNg secrection to H1703 A2+ was lower than A2 KO (lower efficacy).

Killing and IFNg secretion of A2+ and A2− cells by VR51 CD8 cells was analyzed in FIG. 19. The results show that there was a clear relationship between killing and IFNg secretion, as the killing and IFNg secretion increased at similar effector versus target ratios. They also show that the protection of IFNg to H1703 A2+ was strong but not complete.

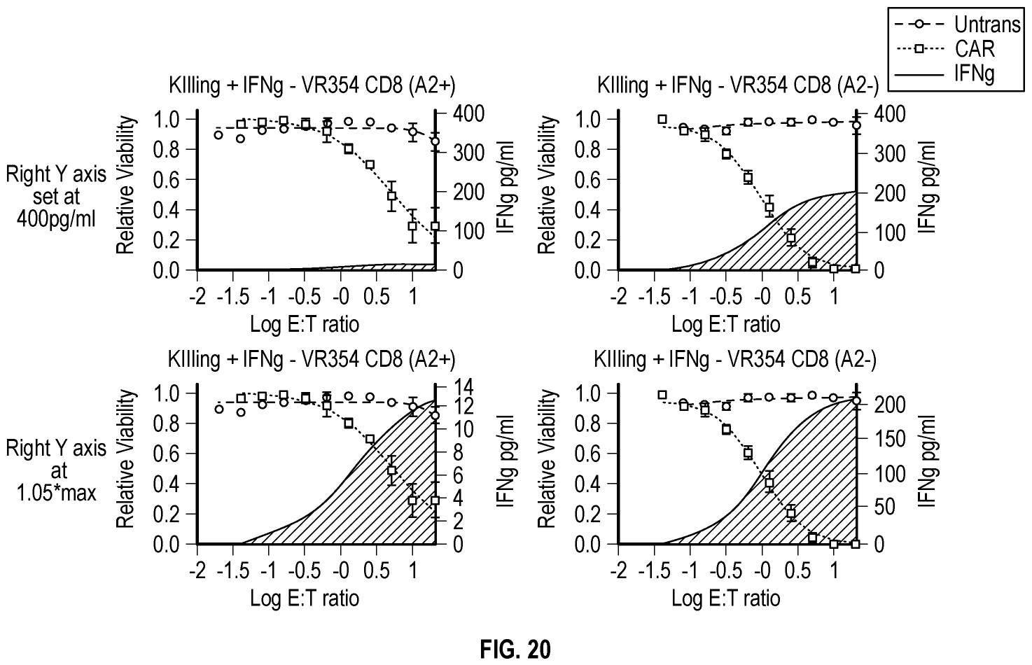

Killing and IFNg secretion of A2+ and A2− cells by VR354 CD8 cells was analyzed in FIG. 20. The results show that there was a clear relationship between killing and IFNg secretion, as the killing and IFNg secretion increased at similar effector versus target ratios. They also show that the protection of IFNg to H1703 A2+ was strong but not complete.

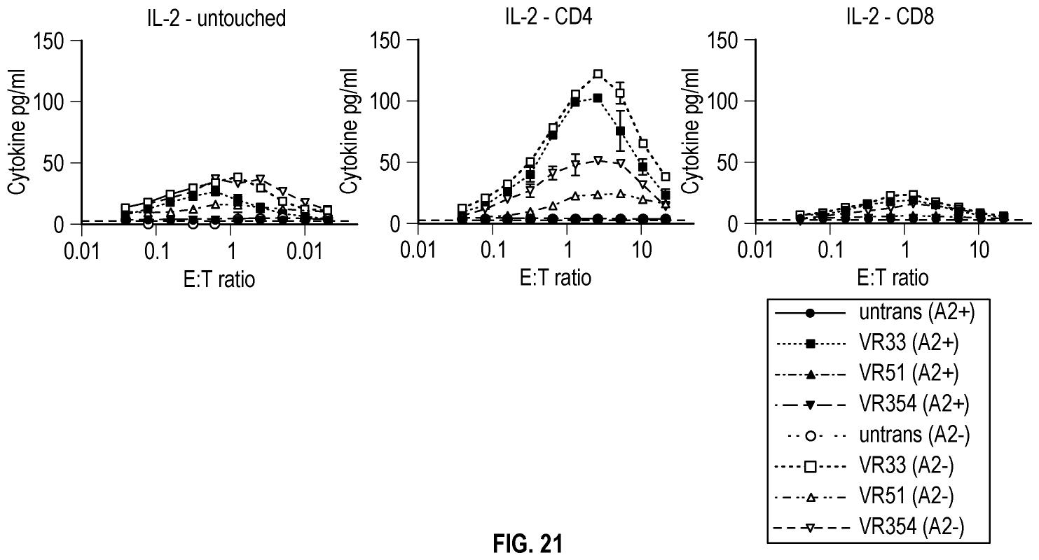

IL2 production for untouched, CD4, and CD8 CAR T cells was analyzed in FIG. 21. The results showed that IL2 was produced mainly by CD4 cells, and protection, indicated by reduced or non cytokine secretion by the T cells, was reduced to background. They also showed that VR33 produce more IL2 than VR51 and VR354 in response to H1703 A2−; IL2 concentration went down at higher effector versus target ratios due to uptake from T cells; and IL2 production was low, especially for untouched and CD8 cells.

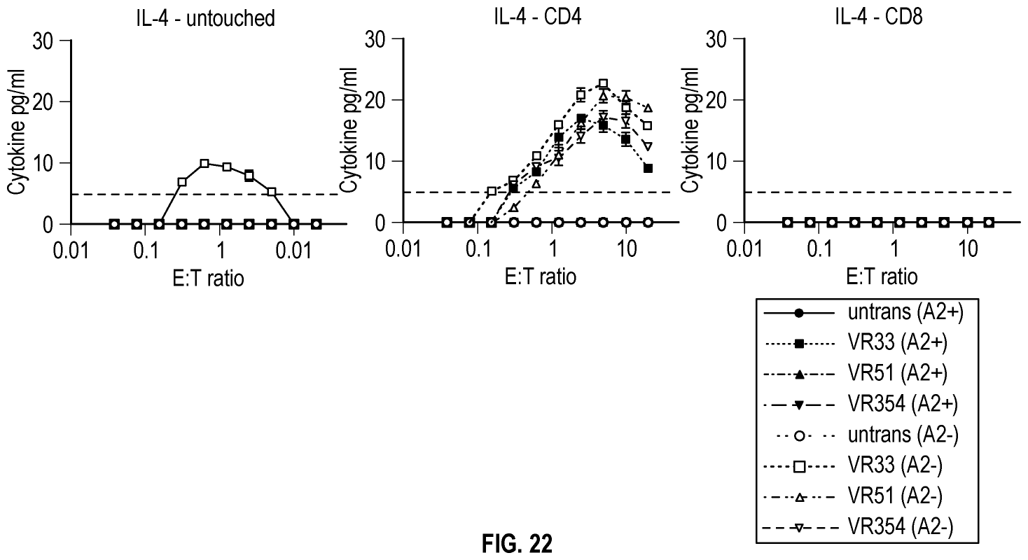

IL4 production for untouched, CD4, and CD8 CAR T cells was analyzed in FIG. 22. The results showed that IL4 was produced mainly by CD4 cells, and protection, indicated by reduced or non cytokine secretion by the T cells, was reduced to background. They also showed that IL4 concentration went down at higher effector versus target ratios due to uptake from T cells; IL4 production was low even in CD4 cells and was not much higher than detection limit; and IL4 production was below detection levels in untouched and CD8 cells in nearly all samples.

### Summary

CD4 CAR T cells were validated for efficacy and conferred complete protection. These findings were consistent across different donors; iCAR scFv, hinge and transmembrane domains, and iDomain; isolation time (day 0, day 14); fresh and thawed effector cells and several target cell lines. See FIGS. 1-22 as well as Tables 1-22 for illustrative design and evaluation of examples of iCAR and aCAR constructs as described herein, as well as sequences thereof, in unsorted/untouched, CD4, and CD8 cells.

All headings and section designations are used for clarity and reference purposes only and are not to be considered limiting in any way. For example, those of skill in the art will appreciate the usefulness of combining various aspects from different headings and sections as appropriate according to the spirit and scope of the invention described herein.

All references cited herein are hereby incorporated by reference herein in their entireties and for all purposes to the same extent as if each individual publication or patent or patent application was specifically and individually indicated to be incorporated by reference in its entirety for all purposes.

Many modifications and variations of this application can be made without departing from its spirit and scope, as will be apparent to those skilled in the art. The specific embodiments and examples described herein are offered by way of example only, and the application is to be limited only by the terms of the appended claims, along with the full scope of equivalents to which the claims are entitled.

1. A population of CD4+ cells, CD8+ cells, or a combination thereof, comprising a bicistronic inhibitory chimeric antigen receptor (iCAR)/activating chimeric antigen receptor (aCAR) nucleotide construct which encodes:
i. an iCAR portion, comprising:
a. an iCAR single chain variable fragment (scFv) component optionally in the VH-VL or VL-VH orientation, comprising a first linker, wherein the iCAR targets a first antigen;
b. an iCAR hinge domain component;
c. an iCAR transmembrane (TM) domain component;
d. an iCAR inhibitory domain component; and
ii. an aCAR portion, comprising:
e. an aCAR single chain variable fragment (scFv) component, optionally in the VH-VL or VL-VH orientation, comprising a second linker, wherein the aCAR scFv targets a second antigen;
f. an aCAR hinge domain component;
g. an aCAR transmembrane (TM) domain component;
h. an aCAR co-stimulatory domain component
i. an aCAR activation signaling domain; and
iii. the bicistronic construct comprises a third linker that connects the iCAR portion in (i) and the aCAR portion in (ii).

2. The population of CD4+ cells, CD8+ cells, or combination thereof, according to claim 1, wherein the first and/or second linker comprises one or more linkers selected from the group consisting of: (G4S)X3 linker (SEQ ID NO:81), G4S linker (SEQ ID NO: 153), (G4S)X3 linker (SEQ ID NO:154), and Whitlow linker (SEQ ID NO: 82).

3. The population of CD4+ cells, CD8+ cells, or combination thereof, according to claim 2, wherein the iCAR scFv component targets an HLA antigen.

4. The population of CD4+ cells, CD8+ cells, or combination thereof, according to claim 3, wherein the HLA antigen consists essentially of or is HLA-A2, HLA-A3, HLA-A, HLA-B, HLA-C, HLA-G, HLA-E, HLA-F, HLA-DPA1, HLA-DQA1, HLA-DQB1, HLA-DQB2, HLA-DRB1, and HLA-DRB5.

5. The population of CD4+ cells, CD8+ cells, or combination thereof, according to claim 4, wherein the iCAR scFv component is selected from the group consisting of: BB7.2, 3PF12, 3PF12/C4, 3PF12/F12, 3PF12/B11, W6/32, BBM.1, SN66E3, Ha5C2.A2, MWB1, MWB1-mod, Hz.BB7.2 VH1-69_A18VK, Hz.BB7.2 VH1-69 (27,30)_A18, Hz.BB7.2 VH1-69 (27,30,48)_A18, Hz.BB7.2 VH1-69 (27,30,67)_A18, Hz.BB7.2 VH1-69 (27,30,69)_A18, Hz.BB7.2 VH1-69 (27,30,67,69)_A18, Hz.BB7.2 VH1-3_A18, Hz.BB7.2 VH1-3(48)_A18, Hz.BB7.2 VH1-3(67)_A18, Hz.BB7.2 VH1-3(69)_A18, Hz.BB7.2 VH1-3(71)_A18, Hz.BB7.2 VH1-3(73)_A18, MWB1.2, SN66E3.2 and SN66E3.3.

6. The population of CD4+ cells, CD8+ cells, or combination thereof, according to claim 4, wherein the iCAR scFv component comprises or consists essentially of (i) Hz BB7.2.1 (SEQ ID NO:287), or (ii) SN66E3.3 (SEQ ID NO:286).

7. (canceled)

8. (canceled)

9. (canceled)

10. The population of CD4+ cells, CD8+ cells, or combination thereof, according to claim 6, wherein the iCAR hinge domain component is selected from a LIR1 52 aa hinge, a LIR1 36 aa hinge, a LIR1 30 aa hinge, a LIR1 26 aa hinge, or a LIR1 8 aa hinge.

11. (canceled)

12. The population of CD4+ cells, CD8+ cells, or combination thereof, according to claim 10, wherein the iCAR inhibitory domain component is an inhibitory domain from a protein selected from the group consisting of LIR1, LIR2, LIR3, LIR5, or LIR8.

13. (canceled)

14. The population of CD4+ cells, CD8+ cells, or combination thereof, according to claim 12, wherein the iCAR inhibitory domain component is a LIR1 inhibitory domain (SEQ ID NO:143).

15. (canceled)

16. The population of CD4+ cells, CD8+ cells, or combination thereof, according to claim 14, wherein the aCAR scFv comprises or consists of the VH and VL domains from trastuzumab (SEQ ID NOs:170 and 171, respectively).

17. (canceled)

18. The population of CD4+ cells, CD8+ cells, or combination thereof, according to claim 16, wherein the aCAR scFv comprises or consists essentially of the VH and VL domains of SEQ ID NO:172, in the VL-VH orientation.

19. (canceled)

20. The population of CD4+ cells, CD8+ cells, or combination thereof, according to claim 18, wherein the aCAR hinge TM domain component consists essentially of or is a CD8 alpha hinge domain (SEQ ID NO:84).

21. The population of CD4+ cells, CD8+ cells, or combination thereof, according to claim 20, wherein the aCAR co-stimulatory domain component is selected from the group consisting of a CD137 (4-1BB) co-stimulatory domain, a CD28 co-stimulatory domain, a 28BB co-stimulatory domain, and a CD3z co-stimulatory domain.

22. The population of CD4+ cells, CD8+ cells, or combination thereof, according to claim 21, wherein the aCAR co-stimulatory domain component comprises a CD137 (4-1BB) co-stimulatory domain (SEQ ID NO:233) and/or a CD3z activation signaling domain (SEQ ID NO:235).

23. The population of CD4+ cells, CD8+ cells, or combination thereof, according to claim 22, wherein the aCAR co-stimulatory domain component selected from the group consisting of (i) a component which consists essentially of or is a CD137 (4-1BB) co-stimulatory domain (SEQ ID NO:233) and (ii) a component which consists essentially of or is a CD3z activation signaling domain (SEQ ID NO:235).

24. (canceled)

25. The population of CD4+ cells, CD8+ cells, or combination thereof, according to claim 22, wherein the linker connecting the iCAR portion and the aCAR portion is encoded by an nucleotide sequence that comprises or consists essentially of or is: a T2A sequence (SEQ ID NO:155) or an IRES sequence (SEQ ID NO:159 or 160).

26. The population of CD4+ cells, CD8+ cells, or combination thereof, according to claim 23, wherein the linker connecting the iCAR portion and the aCAR portion is encoded by a nucleotide sequence that comprises or consists essentially of or is an IRES sequence (SEQ ID NO: 159).

27. The population of CD4+ cells, CD8+ cells, or combination thereof, according to claim 1, wherein the bicistronic iCAR/aCAR construct comprises or consists essentially of the nucleic acid sequence selected from the group consisting of: SEQ ID NO:277 and SEQ ID NO:279.

28. (canceled)

29. The population of CD4+ cells, CD8+ cells, or combination thereof, according to claim 27, wherein the bicistronic iCAR/aCAR construct further comprises or consists essentially of: a nucleotide sequence as set forth in one or more of: SEQ ID NO:240, SEQ ID NO:241 or SEQ ID NO:242.

30. The population of CD4+ cells, CD8+ cells, or combination thereof, according to claim 29, wherein the iCAR/aCAR construct further comprises a nucleotide sequence that encodes a CD8 alpha signal peptide as set forth in (SEQ ID NO: 161).
